(19) United States
(12) Patent Application Publication
(10) Pub. No.: US 2026/0327191 A1
(43) Pub. Date: Oct. 22, 2026

(54) GSK3a INHIBITORS AND METHODS OF USE THEREOF

(71) Applicant: Blueprint Medicines Corporation, Cambridge, MA (US)

(72) Inventors: Jason D. Brubaker, Cambridge, MA (US); Michael J. Burke, Cambridge, MA (US); Thomas A. Dineen, Arlington, MA (US); Meredith Suzanne Eno, Cambridge, MA (US); Jie Ge, Cambridge, MA (US); Brian J. Groendyke, Cambridge, MA (US); Stefan David Gross, Cambridge, MA (US); Joseph L. Kim, Cambridge, MA (US); Aysegul Ozen, Cambridge, MA (US); Steven Mark Wenglowsky, Cambridge, MA (US)

(21) Appl. No.: 19/482,336

(22) PCT Filed: May 10, 2024
(86) PCT No.: PCT/US2024/028806

§ 371 Date: Nov. 7, 2025

Related U.S. Application Data
(60) Provisional application No. 63/465,428, filed on May 10, 2023.
(60) Provisional application No. 63/530,858, filed on Aug. 4, 2023.

(51) Int. Cl. C07D 239/47; A61K 31/513; A61K 31/5377; A61K 31/55; A61K 31/553; A61P 35/00; C07B 59/00; C07D 401/12; C07D 401/14; C07D 403/12; C07D 405/14; C07D 413/12; C07D 413/14; C07D 471/10

(52) U.S. Cl. CPC C07D 239/47; A61K 31/513; A61K 31/5377; A61K 31/55; A61K 31/553; A61P 35/00; C07B 59/002; C07D 401/12; C07D 401/14; C07D 403/12; C07D 405/14; C07D 413/12; C07D 413/14; C07D 471/10

(57) ABSTRACT

Disclosed are compounds and compositions which inhibit GSK3α, represented by the Formula (I′) or (X): or a pharmaceutically acceptable salt thereof. The variables in Formula (I′) or (X) are described herein. Also disclosed are methods of treating cancer in a subject with the disclosed GSK3α inhibitors.

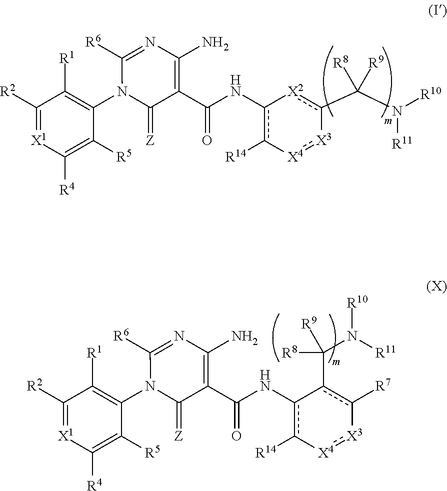

### RELATED APPLICATIONS

This application claims the benefit of U.S. Provisional Application No. 63/465,428 filed on May 10, 2023, and U.S. Provisional Application No. 63/530,858, filed Aug. 4, 2023, the entire teachings of which are incorporated herein by reference in their entirety.

### FIELD

This application is directed to GSK3α-selective inhibitors and methods for their use, such as in enhancing anti-tumor immunity in a subject.

### BACKGROUND

Glycogen synthase kinase 3 (GSK3) is a ubiquitously expressed serine/threonine kinase that is encoded by two genes, GSK3α and GSK3B, first cloned by Woodgett J R [Woodgett J R, EMBO J 1990; 9 (8): 2431-8]. GSK3 kinases are key regulators of cellular signaling pathways downstream of PI3K/mTOR/AKT. GSK3 kinases phosphorylate a broad range of substrates including several transcription factors, for example NFAT [Beals C R, et. al., Science 1997; 275 (5308): 1930-4], and c-Jun [Wei W, et. al., Cancer Cell 2005; 8 (1): 25-33]. In many cases, phosphorylation of substrates by GSK3 leads to subsequent ubiquitination by ubiquitin E3 ligase (e.g., FBXW7 or β-TrCP) followed by proteasomal degradation. Conversely, GSK3 inhibition can lead to stabilization and nuclear translocation of transcription factors that can then activate multiple downstream pathways.

Given the important roles that GSK3 plays in various cellular processes, targeting of GSK3 with small molecule inhibitors may have therapeutic potential for different diseases including but not limited to diabetes, cancer, viral infections, Alzheimer's disease and other CNS disorders. Several molecules have been tested in the clinic, including AZD1080 [Georgievska B, et. al., J Neurochem 2013; 125(3): 446-56], LY2090314 [NCT01287520, NCT01214603], and others. However, dual inhibition of both GSK3α and GSK3β leads to the activation of the Wnt/β-catenin pathway through stabilization of β-catenin, a potent oncogene that is associated with the development of hyperplasia in preclinical animal models [Hall A P, et. al., Toxicol Pathol 2015; 43(3): 384-99].

Although the two isoforms GSK3α and GSK3B were initially thought to be functionally redundant, genetic studies have revealed paralog-specific functions. Notably, while genetic knockout of GSK3B in mice is lethal [Hoeflich K P, et. al., Nature 2000; 406(6791): 86-90], GSK3α knockout mice are viable [Zhou J, et. al., J Clin Invest 2013; 123(4): 1821-32]. GSK3α knockout mice exhibit mild aging phenotypes related to blockade of autophagy and mTOR pathway activation, which could be reversed by treatment with mTORC1 inhibitor everolimus, an immune suppressive drug. Selective, acute inhibition of either GSK3α or GSK3B in mouse embryonic stem cells (ESCs) also lead to distinct morphological phenotypes and transcriptional profiles [Chen X, et. al., Dev Cell 2017; 43(5): 563-576.e4]. Furthermore, Doble et. al. reported that deletion of either GSK3α or GSK3β alone in murine embryonic stem cells (ESCs) does not lead to β-catenin accumulation, while deletion of both paralogs is required to stabilize β-catenin, consistent with observations with dual GSK3 inhibitors [Doble B W, et. al., Dev Cell 2007; 12(6): 957-71]. These lines of evidence suggest that paralog-selective inhibition of GSK3α could have different pharmacology and better safety/tolerability than dual GSK3 inhibition. There are earlier reports toward such GSK3α-selective compounds, however, the degree of reported selectivity is limited, in the range of 3 to 40-fold [US20220112216A1, Wagner F F, et. al, Sci Transl Med 2018; 10(431):eaam8460; Amaral B, et al., ACS Chem. Neurosci. 2023; 14(6): 1080-94].

T cells are critical for immune surveillance and control of cancer progression. It is well known that GSK3 inhibition bypasses the requirement for CD28-mediated co-stimulation of T cells, leading to proliferation and production of cytokines such as IL2 and IFNγ [Garcia C A, et. al., J Immunol 2008; 181(12): 8363-71]. Dual GSK3 inhibitors have been identified in a phenotypic screen of a highly annotated kinase inhibitor library for enhancer of antigen-specific T cell activation and cytotoxicity toward tumor cells. Inhibition of GSK3α instead of GSK3β may be responsible for anti-tumor immunity, and inhibition of GSK3α may be well-tolerated. This hypothesis is supported by work from Pamela Ohashi's lab [Tran C W, et. al., J Immunol 2017; 199(12): 4056-4065] demonstrating that deletion of GSK3α in CD4 T cells reduces the level of negative T cell regulator Cbl-b post T cell stimulation. Additional literature supporting selective targeting of GSK3α in immune and cancer cells come from works of Christopher E Rudd [Taylor A, et. al., Immunity 2016; 44(2): 274-86] and Alejandro Gutierrez [Hinze L, et. al., Cancer Cell 2019; 35(4): 664-676.e7], through other proposed mechanisms.

Herein, the identification of paralog-selective inhibitors of GSK3α and their use as single agents or in combination with anti-PD-1 or anti-PD-L1 in cancer immunotherapy is described. Without wishing to be bound by any theory, the mechanism, while still under further investigation, is thought to be mediated via both T cells and other immune cell types (e.g., myeloid compartment) in the tumor microenvironment. GSK3α-selective inhibitors may be useful as novel therapies for treating cancer and other diseases.

### SUMMARY

Provided herein are compounds and compositions which inhibit GSK3, more specifically, GSK3α, thereby enhancing an immune response in a subject. For example, the IC₅₀ values for inhibition of GSK3α provided in Table 1 demonstrate that these compounds are potent inhibitors of GSK3α. Compounds provided herein are selective inhibitors of GSK3α. Also disclosed are methods of using the compounds and compositions described herein for treating cancer.

In the first aspect, the present disclosure provides a compound of Formula (I′) or Formula (X):

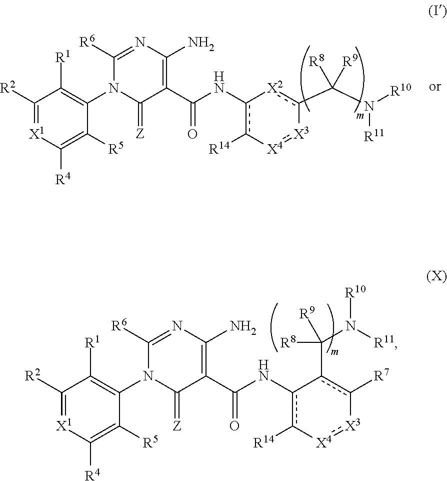

or a pharmaceutically acceptable salt thereof, wherein:
[symbol] is a single bond or a double bond;
X¹ is CR³ or N;
X² is selected from the group consisting of CR⁷, N, and NR⁴,
X³ is selected from the group consisting of CR¹², N, and NR⁴;
X⁴ is selected from the group consisting of CR¹³, N, and NR^{d};
Z is O or S;
R¹ is selected from the group consisting of halo, OH, CN, C₁-C₄alkyl, and C₁-C₄alkoxy, wherein the C₁-C₄alkyl and C₁-C₄alkoxy are each optionally substituted with 1 to 4 groups each independently selected from the group consisting of halo, OH and CN;
R² is selected from the group consisting of H, D, halo, C₁-C₄alkyl, and C₃-C₁₀cycloalkyl;
R³ is selected from the group consisting of H, D, halo, CN, C₁-C₄alkyl, —(C(R^{a})₂)ₙ—OR^{b}, —(C(R^{a})₂)ₙ—C(O)OR^{b}, —(C(R^{a})₂)ₙ—SO₂—R^{b}, —N(R^{a})₂, C₃-C₁₀cycloalkyl, 4 to 12-membered heterocyclyl, 4 to 12-membered aryl, and 4 to 12-membered heteroaryl, wherein the C₁-C₄alkyl, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c};
R⁴ is selected from the group consisting of H, D, halo, C₁-C₄alkyl, and C₃-C₁₀cycloalkyl;
R⁵ is selected from the group consisting of halo, OH, CN, C₁-C₄alkyl, and C₁-C₄alkoxy, wherein the C₁-C₄alkyl and C₁-C₄alkoxy each optionally substituted with 1 to 4 groups each independently selected from the group consisting of halo, OH and CN;
R⁶ is H or D;
R⁷ is selected from the group consisting of H, D, halo, C₁-C₄alkyl, C₁-C₄alkoxy, C₃-C₁₀cycloalkyl, 4 to 12-membered heterocyclyl, 4 to 12-membered aryl, and 4 to 12-membered heteroaryl, wherein the C₁-C₄alkyl, C₁-C₄alkoxy, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c};
R⁸ is selected from the group consisting of H, D, halo, C₁-C₄alkyl, and C₃-C₁₀cycloalkyl; R⁹ are each independently selected from the group consisting of H, D, C₁-C₄alkyl, C₂-C₄alkenyl, C₂-C₄alkynyl, —(C(R^{a})₂)ₙ—OR^{b}, C₃-C₁₀cycloalkyl, 4 to 12-membered heterocyclyl, 4 to 12-membered aryl, and 4 to 12-membered heteroaryl, wherein the C₁-C₄alkyl, C₂-C₄alkenyl, C₂-C₄alkynyl, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c}; or
R⁷ and R⁹ are taken together with the carbon atoms to which they are attached to form Ring A, wherein Ring A is C₃-C₁₀cycloalkyl or 4 to 12-membered heterocyclyl, wherein the C₃-C₁₀cycloalkyl is optionally substituted with 1 to 4 R^{c}, wherein the 4 to 12-membered heterocyclyl has 1 to 4 ring heteroatoms each independently selected from the group consisting of O, S, and NR^{d}, and then is optionally substituted on a ring carbon by 1 to 4 R^{c}; or
R⁸ and R⁹ are taken together with the carbon atom to which they are attached to form Ring B, wherein Ring B is C₃-C₁₀cycloalkyl or 4 to 12-membered heterocyclyl, wherein the C₃-C₁₀cycloalkyl is optionally substituted with 1 to 4 R^{c}, wherein the 4 to 12-membered heterocyclyl has 1 to 4 ring heteroatoms each independently selected from the group consisting of O, S, and NR^{d}, and then is optionally substituted on a ring carbon by 1 to 4 R^{c}; or
R⁸ and R⁹ are taken together form a ═O;
R¹⁰ is selected from the group consisting of H, D, C₁-C₄ alkyl, —(C(R^{a})₂)ₙ—OR^{b}, —(C(R^{a})₂)ₙ—SO₂R^{b}, C₃-C₁₀cycloalkyl, 4 to 12-membered heterocyclyl, 4 to 12-membered aryl, and 4 to 12-membered heteroaryl, wherein the C₁-C₄alkyl, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c}; or
R⁹ and R¹⁰ are taken together with the carbon atom and the nitrogen atom to which they are attached, respectively, to form Ring C, wherein Ring C is a 4 to 12-membered heterocyclyl having 1 to 4 ring heteroatoms each independently selected from the group consisting of O, S, and NR^{d}, and then is optionally substituted on a ring carbon by 1 to 4 R^{c}; or
R⁷ and R⁹ are taken together with the carbon atoms to which they are attached to form Ring A, and R⁸ and R¹⁰ are taken together with the carbon atom and the nitrogen atom to which they are attached, respectively, to form Ring C, wherein Ring A and Ring C are each optionally substituted on a ring carbon by 1 to 4 R^{c};
R¹¹ is selected from the group consisting of H, D, C₁-C₄alkyl, C₁-C₄haloalkyl, and C₃-C₁₀cycloalkyl;
R¹² is selected from the group consisting of H, D, halo, C₁-C₄alkyl, and C₃-C₁₀cycloalkyl; or R¹¹ and R¹² are taken together with the nitrogen atom and the carbon atom to which they are attached, respectively, to form Ring D, wherein Ring D is selected from the group consisting of a 4 to 12-membered heterocyclyl 4 to 12-membered heteroaryl, wherein the 4 to 12-membered heterocyclyl and 4 to 12-membered heteroaryl have 1 to 4 ring heteroatoms each independently selected from the group consisting of O, S, N, and NR^{d}, and then are each optionally substituted on a ring carbon by 1 to 4 R^{c};
R¹³ is selected from the group consisting of H, D, halo, CN, C₁-C₄alkyl, C₁-C₄alkoxy, C₃-C₁₀cycloalkyl, 4 to 12-membered heterocyclyl, 4 to 12-membered aryl, and 4 to 12-membered heteroaryl, wherein the C₁-C₄alkyl, C₁-C₄alkoxy, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c};
R¹⁴ is selected from the group consisting of H, D, and C₁-C₄alkyl;
Each R^{a} is independently selected from the group consisting of H, D, halo, CN, C₁-C₄alkyl, and C₁-C₄alkoxy, wherein the C₁-C₄alkyl and C₁-C₄alkoxy each optionally substituted with 1 to 4 groups each independently selected from the group consisting of halo, OH and CN;
Each R^{b} is independently selected from the group consisting of H, D, C₁-C₄alkyl, C₃-C₁₀cycloalkyl, 4 to 12-membered heterocyclyl, 4 to 12-membered aryl, and 4 to 12-membered heteroaryl, wherein the C₁-C₄alkyl, C₃-C₁₀cycloalkyl, and 4 to 12-membered aryl are each optionally substituted with 1 to 4 groups each independently selected from the group consisting of halo, OH and CN, wherein the 4 to 12-membered heterocyclyl and 4 to 12-membered heteroaryl have 1 to 4 ring heteroatoms each independently selected from the group consisting of O, S, N, and NR^{d} and then are optionally substituted on a ring carbon with 1 to 4 groups each independently selected from the group consisting of halo, OH and CN;
Each R^{c} is independently selected from the group consisting of H, D, halo, OH, CN, C₁-C₄alkyl, and C₁-C₄alkoxy, or two R^{c}, attached to the same atom, form a ═O, wherein said C₁-C₄alkyl and C₁-C₄alkoxy are each optionally substituted with 1 to 4 groups each independently selected from the group consisting of halo, OH and CN;
Each R^{d} is independently selected from the group consisting of H, D, C₁-C₄alkyl, and C(O)C₁-4alkyl; and
m is 1, 2, or 3, and
n is 0, 1, 2, or 3.

Another aspect of the disclosure is a pharmaceutical composition comprising a pharmaceutically acceptable carrier, excipient, or diluent and a compound disclosed herein, or a pharmaceutically acceptable salt thereof.

Another aspect of the disclosure is a method of treating a disease or disorder responsive to inhibition of GSK3 (e.g., GSK3α), comprising administering to the subject an effective amount of a compound disclosed herein or a pharmaceutically acceptable salt thereof, or a pharmaceutical composition comprising the compound(s) or a pharmaceutically acceptable salt thereof. In some embodiments, the disease or disorder is a cancer.

Another aspect of the disclosure is the use of a compound disclosed herein or a pharmaceutically acceptable salt thereof, or a pharmaceutical composition comprising the compound(s) or a pharmaceutically acceptable salt thereof, for the preparation of a medicament for treating a disease or disorder responsive to inhibition of GSK3 (e.g., GSK3α). In some embodiments, the disease or disorder is a cancer.

Another aspect of the disclosure is a compound disclosed herein or a pharmaceutically acceptable salt thereof, or a pharmaceutical composition comprising the compound(s) or a pharmaceutically acceptable salt thereof, for use in treating a disease or disorder responsive to inhibition of GSK3 (e.g., GSK3α). In some embodiments, the disease or disorder is a cancer.

### BRIEF DESCRIPTION OF THE FIGURES

FIG. 1 shows tumor volume in an MC38 mouse model of colorectal cancer with treatment with anti-PD-L1 antibody, Compound 178 or a combination of anti-PD-L1 antibody and Compound 178.

### DETAILED DESCRIPTION

The disclosed compounds are GSK3α inhibitors, which can be used for treating a disease or disorder responsive to inhibition of GSK3α. Such diseases or disorders include cancer.

### COMPOUND EMBODIMENTS

Example embodiments include:

### First Embodiment

a compound represented by Formula (I):

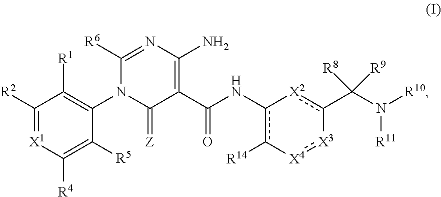

or a pharmaceutically acceptable salt thereof. The variables in Formula (I) are as described above for Formula (I′) in the first aspect.

### Second Embodiment

a compound represented by Formula (II):

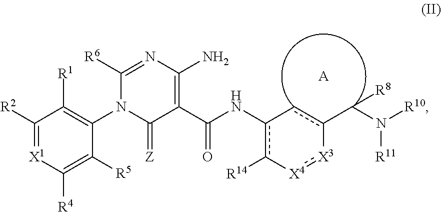

or a pharmaceutically acceptable salt thereof, wherein the definitions for the variables in Formula (II) are as defined in the first embodiment.

### Third Embodiment

a compound represented by Formula (IIa), (IIb) or (IIc):

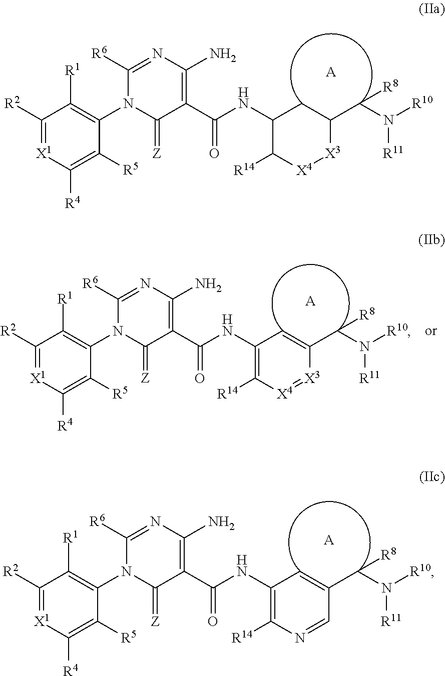

or a pharmaceutically acceptable salt thereof, wherein the definitions for the variables in Formulae (IIa), (IIb) and (IIc) are as defined in the first embodiment.

### Fourth Embodiment

a compound represented by Formula (I′), (I), (X), (IIa), (IIb) or (IIc), or a pharmaceutically acceptable salt thereof, wherein Ring A is C₃-C₆ cycloalkyl or 4 to 9-membered heterocyclyl, wherein the C₃-C₆ cycloalkyl is optionally substituted with 1 to 2 R^{c}, wherein the 4 to 9-membered heterocyclyl has 1 to 2 ring heteroatoms each independently selected from the group consisting of O, S, and NR^{d}, and then is optionally substituted on a ring carbon by 1 to 2 R^{c}. The remainder of the variables in Formula (I′), (I), (X), (IIa), (IIb) or (IIc) are as defined in the first embodiment. In some embodiments, the compound is represented by Formula (IIc) or a pharmaceutically acceptable salt thereof.

### Fifth Embodiment

a compound represented by Formula (I′), (I), (X), (IIa), (IIb) or (IIc), or a pharmaceutically acceptable salt thereof, wherein Ring A is represented by

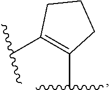 [SMILES: *C1=C(*)CCC1]

and the remainder of the variables in Formula (I′), (I), (X), (IIa), (IIb) and (IIc) are as defined in the first or fourth embodiment. In some embodiments, the compound is represented by Formula (IIc) or a pharmaceutically acceptable salt thereof.

### Sixth Embodiment

A Compound Represented by Formula (III):

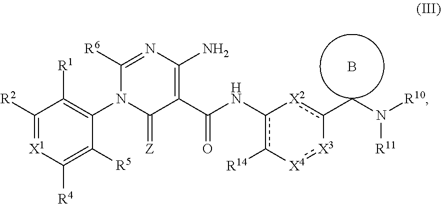

or a pharmaceutically acceptable salt thereof, wherein the definitions for the variables in Formula (III) are as defined in the first embodiment.

### Seventh Embodiment

a compound represented by Formula (IIIa) or (IIIb):

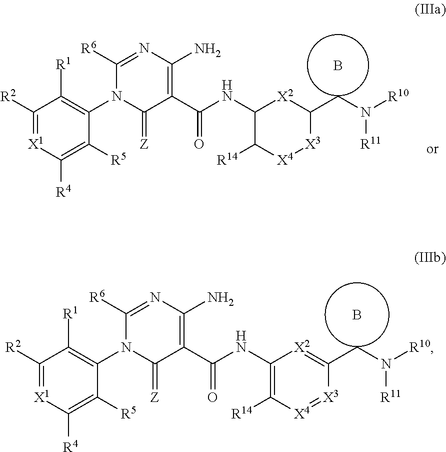

or a pharmaceutically acceptable salt thereof, wherein the definitions for the variables in Formulae (IIIa) and (IIIb) are as defined in the first embodiment.

### Eighth Embodiment

a compound represented by Formula (I′), (I), (X), (III), (IIIa) or (IIIb), or a pharmaceutically acceptable salt thereof, wherein Ring B is C₃-C₆ cycloalkyl or 4 to 6-membered heterocyclyl, wherein the C₃-C₆ cycloalkyl is optionally substituted with 1 to 4 R^{c}, wherein the 4 to 6-membered heterocyclyl has 1 to 2 ring heteroatoms each independently selected from the group consisting of O, S, and NR^{d}, and then is optionally substituted on a ring carbon by 1 to 2 R^{c}. The remainder of the variables in Formulae (I′), (I), (X), (III), (IIIa) and (IIIb) are as defined in the first aspect or first embodiment.

### Ninth Embodiment

a compound represented by Formula (I′), (I), (X), (III), (IIIa) or (IIIb), or a pharmaceutically acceptable salt thereof, wherein Ring B is cyclopropane, cyclobutane, cyclopentane, or tetrahydropyran, each of which is optionally substituted on a ring carbon by 1 to 2 R^{c}. The remainder of the variables in Formulae (I′), (I), (X), (III), (IIIa) and (IIIb) are as defined in the first aspect or first embodiment.

### Tenth Embodiment

a compound represented by Formula (IV):

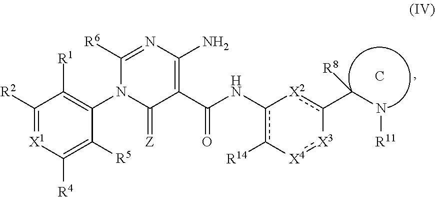

or a pharmaceutically acceptable salt thereof, wherein the definitions for the variables in Formula (IV) are as defined in the first embodiment.

### Eleventh Embodiment

a compound represented by Formula (IVa), (IVb), (IVc) or (IVd):

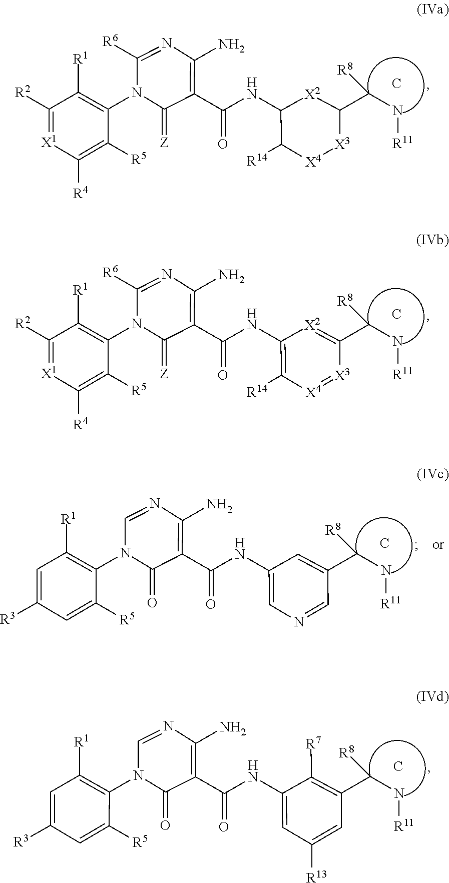

or a pharmaceutically acceptable salt thereof, wherein the definitions for the variables in Formulae (IVa), (IVb), (IVc) and (IVd) are as defined in the first embodiment. In some embodiments, the compound is represented by Formula (IVc) or (IVd) or a pharmaceutically acceptable salt thereof.

### Twelfth Embodiment

a compound represented by Formula (I′), (I), (X), (IV), (IVa), (IVb), (IVc) or (IVd), or a pharmaceutically acceptable salt thereof, wherein Ring C is a 4 to 10-membered heterocyclyl having 1 to 3 ring heteroatoms each independently selected from the group consisting of O, S, and NR^{d}, and then is optionally substituted on a ring carbon by 1 to 2 R^{c}. The remainder of the variables in Formulae (I′), (I), (X), (IV), (IVa), (IVb), (IVc) and (IVd) are as defined in the first embodiment. In some embodiments, the compound is represented by Formula (IVc) or (IVd) or a pharmaceutically acceptable salt thereof.

### Thirteenth Embodiment

a compound represented by Formula (I′), (I), (X), (IV), (IVa), (IVb), (IVc) or (IVd), or a pharmaceutically acceptable salt thereof, wherein Ring C is piperidine, 1,4-oxazepane, azetidine, morpholine, pyrrolidine, piperazine, piperazine-2-one, octahydrocyclopenta[c]pyrrole, or azepane, each of which is optionally substituted on a ring carbon by 1 to 2 R^{c}. The remainder of the variables in Formulae (I′), (I), (X), (IV), (IVa), (IVb), (IVc) and (IVd) are as defined in the first embodiment. In some embodiments, the compound is represented by Formula (IVc) or (IVd) or a pharmaceutically acceptable salt thereof.

### Fourteenth Embodiment

a compound represented by Formula (I′), (I), (X), (IV), (IVa), (IVb), (IVc) or (IVd), or a pharmaceutically acceptable salt thereof, wherein Ring C is represented by

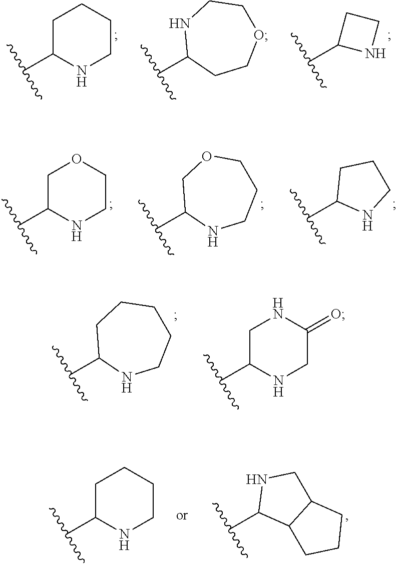

each of which is optionally substituted with 1 or 2 R^{c}; and each Re is independently C₁-C₃alkyl optionally substituted with halo or OH. Alternatively, Re is —CH₃ or —CH₂OH. The remainder of the variables in Formulae (I′), (I), (X), (IV), (IVa), (IVb), (IVc) and (IVd) are as defined in the first embodiment. In some embodiments, the compound is represented by Formula (IVc) or (IVd) or a pharmaceutically acceptable salt thereof.

### Fifteenth Embodiment

a compound represented by Formula (V):

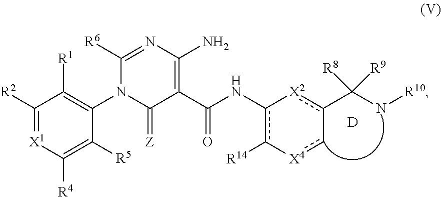

or a pharmaceutically acceptable salt thereof, wherein the definitions for the variables in Formula (V) are as defined in the first embodiment.

### Sixteenth Embodiment

a compound represented by Formula (Va) or (Vb):

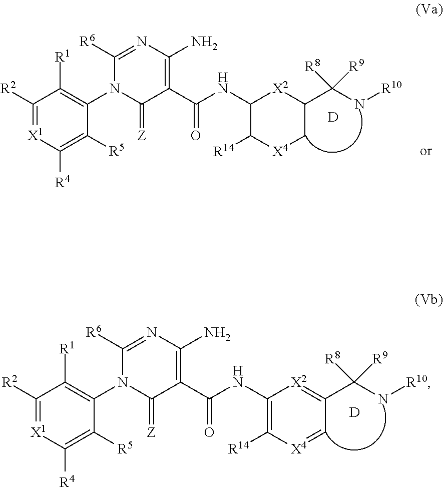

or a pharmaceutically acceptable salt thereof, wherein the definitions for the variables in Formulae (Va) and (Vb) are as defined in the first embodiment.

### Seventeenth Embodiment

a compound represented by Formula (VI):

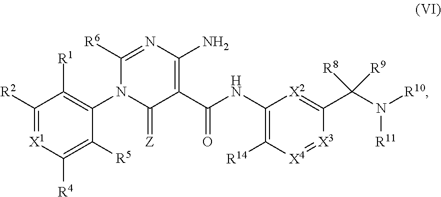

or a pharmaceutically acceptable salt thereof, wherein the definitions for the variables in Formula (VI) are as defined in the first embodiment.

### Eighteenth Embodiment

a compound represented by Formula (VI), or a pharmaceutically acceptable salt thereof, wherein:
X¹ is CR³ or N;
X² is CR⁷ or N;
X³ is CR¹² or N;
X⁴ is CR¹³ or N;
Z is O or S;
R¹ is selected from the group consisting of halo, OH, CN, C₁-C₄alkyl, C₁-C₄alkoxy, and C₁-C₄haloalkyl;
R² is selected from the group consisting of H, D, C₁-C₄alkyl, and C₃-C₁₀cycloalkyl;
R³ is selected from the group consisting of H, D, halo, CN, C₁-C₄alkyl, —(C(R^{a})₂)ₙ—OR^{b}, —(C(R^{a})₂)ₙ—C(O)OR^{b}, —(C(R^{a})₂)ₙ—SO₂—R^{b}, —N(R^{a})₂, C₃-C₁₀cycloalkyl, 4 to 12-membered heterocyclyl, 4 to 12-membered aryl, and 4 to 12-membered heteroaryl, wherein the C₁-C₄alkyl, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c};
R⁴ is selected from the group consisting of H, D, C₁-C₄alkyl, and C₃-C₁₀cycloalkyl;
R⁵ is selected from the group consisting of halo, OH, CN, C₁-C₄alkyl, C₁-C₄alkoxy, and C₁-C₄haloalkyl;
R⁶ is H or D;
R⁷ is selected from the group consisting of H, D, halo, C₁-C₄alkyl, C₁-C₄alkoxy, C₃-C₁₀cycloalkyl, and 4 to 12-membered heterocyclyl, wherein the C₁-C₄alkyl, C₁-C₄alkoxy, and C₃-C₁₀cycloalkyl are each optionally substituted with 1 to 4 R^{c}, wherein the 4 to 12-membered heterocyclyl has 1 to 4 ring heteroatoms each independently selected from the group consisting of O, S and NR^{d} and then are each optionally substituted on a ring carbon with 1 to 4 R^{c};
R⁸ is selected from the group consisting of H, D, C₁-C₄alkyl, and C₃-C₁₀cycloalkyl;
R⁹ are each independently selected from the group consisting of H, D, C₁-C₄alkyl, C₂-C₄alkenyl, C₂-C₄alkynyl, —(C(R^{a})₂)ₙ—OR^{b}, C₃-C₁₀cycloalkyl, 4 to 12-membered heterocyclyl, 4 to 12-membered aryl, and 4 to 12-membered heteroaryl, wherein the C₁-C₄alkyl, C₂-C₄alkenyl, C₂-C₄alkynyl, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c}; or
R⁷ and R⁹ are taken together with the carbon atoms to which they are attached to form Ring A, wherein Ring A is C₃-C₁₀cycloalkyl or 4 to 12-membered heterocyclyl, wherein the C₃-C₁₀cycloalkyl is optionally substituted with 1 to 4 R^{c}, wherein the 4 to 12-membered heterocyclyl has 1 to 4 ring heteroatoms each independently selected from the group consisting of O, S, and NR^{d}, and then is optionally substituted on a ring carbon by 1 to 4 R^{c}; and/or
R⁸ and R⁹ are taken together with the carbon atom to which they are attached to form Ring B, wherein Ring B is C₃-C₁₀cycloalkyl or 4 to 12-membered heterocyclyl, wherein the C₃-C₁₀cycloalkyl is optionally substituted with 1 to 4 R^{c}, wherein the 4 to 12-membered heterocyclyl has 1 to 4 ring heteroatoms each independently selected from the group consisting of O, S, and NR^{d}, and then is optionally substituted on a ring carbon by 1 to 4 R^{c}; or
R⁸ and R⁹ are taken together form a ═O;
R¹⁰ is selected from the group consisting of H, D, C₁-C₄ alkyl, —(C(R^{a})₂)ₙ—ORD, —(C(R^{a})₂)ₙ—SO₂R^{b}, C₃-C₁₀cycloalkyl, 4 to 12-membered heterocyclyl, 4 to 12-membered aryl, and 4 to 12-membered heteroaryl, wherein the C₁-C₄alkyl, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c}; or
R⁹ and R¹⁰ are taken together with the carbon atom and the nitrogen atom to which they are attached, respectively, to form Ring C, wherein Ring C is a 4 to 12-membered heterocyclyl having 1 to 4 ring heteroatoms each independently selected from the group consisting of O, S, and NR^{d}, and then is optionally substituted on a ring carbon by 1 to 4 R^{c};
R¹¹ is selected from the group consisting of H, D, C₁-C₄alkyl, and C₃-C₁₀cycloalkyl;
R¹² is selected from the group consisting of H, D, halo, C₁-C₄alkyl, and C₃-C₁₀cycloalkyl;
R¹³ is selected from the group consisting of H, D, halo, CN, C₁-C₄alkyl, C₁-C₄alkoxy, C₃-C₁₀cycloalkyl, 4 to 12-membered heterocyclyl, 4 to 12-membered aryl, and 4 to 12-membered heteroaryl, wherein the C₁-C₄alkyl, C₁-C₄alkoxy, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c};
R¹⁴ is selected from the group consisting of H, D, and C₁-C₄alkyl;
Each R^{a} is independently selected from the group consisting of H, D, halo, CN, C₁-C₄alkyl, and C₁-C₄alkoxy, wherein the C₁-C₄alkyl and C₁-C₄alkoxy each optionally substituted with 1 to 4 groups each independently selected from the group consisting of halo, OH and CN;
Each R^{b} is independently selected from the group consisting of H, D, C₁-C₄alkyl, C₃-C₁₀cycloalkyl, 4 to 12-membered heterocyclyl, 4 to 12-membered aryl, and 4 to 12-membered heteroaryl, wherein the C₁-C₄alkyl, C₃-C₁₀cycloalkyl, and 4 to 12-membered aryl are each optionally substituted with 1 to 4 groups each independently selected from the group consisting of halo, OH and CN, wherein the 4 to 12-membered heterocyclyl and 4 to 12-membered heteroaryl have 1 to 4 ring heteroatoms each independently selected from the group consisting of O, S, N, and NR^{d} and then are optionally substituted on a ring carbon with 1 to 4 groups each independently selected from the group consisting of halo, OH and CN;
Each R^{c} is independently selected from the group consisting of H, D, halo, OH, CN, C₁-C₄alkyl, and C₁-C₄alkoxy, or two R^{c}, attached to the same atom, form a ═O, wherein the C₁-C₄alkyl and C₁-C₄alkoxy are each optionally substituted with 1 to 4 groups each independently selected from the group consisting of halo, OH and CN;
Each R^{d} is independently selected from the group consisting of H, D, C₁-C₄alkyl, and C(O)C₁-4alkyl; and
n is 0, 1, 2, or 3.

### Nineteenth Embodiment

a compound represented by Formula (VI), or a pharmaceutically acceptable salt thereof, wherein:
X¹ is CR³ or N;
X² is CR⁷ or N;
X³ is CR¹² or N;
X⁴ is CR¹³ or N;
Z is O;
R¹ is selected from the group consisting of halo, OH, C₁-C₄alkyl, C₁-C₄alkoxy, and C₁-C₄haloalkyl;
R² is selected from the group consisting of H, D, and C₁-C₄alkyl;
R³ is selected from the group consisting of H, D, halo, CN, C₁-C₄alkyl, —(C(R^{a})₂)ₙ—OR^{b}, —(C(R^{a})₂)ₙ—C(O)OR^{b}, —(C(R^{a})₂)ₙ—SO₂—R^{b}, —N(R^{a})₂, C₃-C₈cycloalkyl, 4 to 10-membered heterocyclyl, 4 to 10-membered aryl, and 4 to 10-membered heteroaryl, wherein the C₁-C₄alkyl, C₃-C₈cycloalkyl, and 4 to 10-membered aryl are each optionally substituted with 1 to 3 R^{c}, wherein the 4 to 10-membered heterocyclyl and 4 to 10-membered heteroaryl have 1 to 3 ring heteroatoms each independently selected from the group consisting of O, S, N, and NR^{d} and then are each optionally substituted on a ring carbon with 1 to 3 R^{c};
R⁴ is selected from the group consisting of H, D, and C₁-C₄alkyl;
R⁵ is selected from the group consisting of halo, OH, C₁-C₄alkyl, C₁-C₄alkoxy, and C₁-C₄haloalkyl;
R⁶ is H or D;
R⁷ is selected from the group consisting of H, D, halo, C₁-C₄alkyl, C₁-C₄alkoxy, C₃-C₈cycloalkyl, and 4 to 10-membered heterocyclyl, wherein the C₁-C₄alkyl, C₁-C₄alkoxy, and C₃-C₈cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are each optionally substituted on a ring carbon with 1 to 3 R^{c};
R⁸ is selected from the group consisting of H, D, and C₁-C₄alkyl;
R⁹ are each independently selected from the group consisting of H, D, C₁-C₄alkyl, C₂-C₄alkenyl, C₂-C₄alkynyl, —(C(R^{a})₂)ₙ—OR^{b}, C₃-C₈cycloalkyl, and 4 to 10-membered heterocyclyl, wherein the C₁-C₄alkyl, C₂-C₄alkenyl, C₂-C₄alkynyl, and C₃-C₈cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are each optionally substituted on a ring carbon with 1 to 3 R^{c}; or
R⁷ and R⁹ are taken together with the carbon atoms to which they are attached to form Ring A, wherein Ring A is C₃-C₈cycloalkyl or 4 to 10-membered heterocyclyl, wherein the C₃-C₈cycloalkyl is optionally substituted with 1 to 3 R^{c}, wherein the 4 to 10-membered heterocyclyl has 1 to 3 ring heteroatoms each independently selected from the group consisting of O, S, and NR^{d}, and then is optionally substituted on a ring carbon by 1 to 3 R^{c}; and/or
R⁸ and R⁹ are taken together with the carbon atom to which they are attached to form Ring B, wherein Ring B is C₃-C₈cycloalkyl or 4 to 10-membered heterocyclyl, wherein the C₃-C₈cycloalkyl is optionally substituted with 1 to 3 R^{c}, wherein the 4 to 10-membered heterocyclyl has 1 to 3 ring heteroatoms each independently selected from the group consisting of O, S, and NR^{d}, and then is optionally substituted on a ring carbon by 1 to 3 R^{c}; or
R⁸ and R⁹ are taken together form a ═O;
R¹⁰ is selected from the group consisting of H, D, C₁-C₄ alkyl, —(C(R^{a})₂)ₙ—OR^{b}, —(C(R^{a})₂)ₙ—SO₂R^{b}, C₃-C₈cycloalkyl, 4 to 10-membered heterocyclyl, 4 to 10-membered aryl, and 4 to 10-membered heteroaryl, wherein the C₁-C₄alkyl, C₃-C₈cycloalkyl, and 4 to 10-membered aryl are each optionally substituted with 1 to 3 R^{c}, wherein the 4 to 10-membered heterocyclyl and 4 to 10-membered heteroaryl have 1 to 3 ring heteroatoms each independently selected from the group consisting of O, S, N, and NR^{d} and then are each optionally substituted on a ring carbon with 1 to 3 R^{c}; or
R⁹ and R¹⁰ are taken together with the carbon atom and the nitrogen atom to which they are attached, respectively, to form Ring C, wherein Ring C is a 4 to 10-membered heterocyclyl having 1 to 3 ring heteroatoms each independently selected from the group consisting of O, S, and NR^{d}, and then is optionally substituted on a ring carbon by 1 to 3 R^{c};
R¹¹ is selected from the group consisting of H, D, and C₁-C₄alkyl;
R¹² is selected from the group consisting of H, D, halo, and C₁-C₄alkyl;
R¹³ is selected from the group consisting of H, D, halo, CN, C₁-C₄alkyl, and C₁-C₄alkoxy, wherein the C₁-C₄alkyl and C₁-C₄alkoxy are each optionally substituted with 1 to 3 R^{c};
R¹⁴ is selected from the group consisting of H, D, and C₁-C₄alkyl;
Each R^{a} is independently selected from the group consisting of H, D, halo, CN, C₁-C₄alkyl, and C₁-C₄alkoxy, wherein the C₁-C₄alkyl and C₁-C₄alkoxy each optionally substituted with 1 to 3 groups each independently selected from the group consisting of halo, OH and CN;
Each R^{b} is independently selected from the group consisting of H, D, C₁-C₄alkyl, C₃-C₈cycloalkyl, 4 to 10-membered heterocyclyl, 4 to 10-membered aryl, and 4 to 10-membered heteroaryl, wherein the C₁-C₄alkyl, C₃-C₈cycloalkyl, and 4 to 10-membered aryl are each optionally substituted with 1 to 3 groups each independently selected from the group consisting of halo, OH and CN, wherein the 4 to 10-membered heterocyclyl and 4 to 10-membered heteroaryl have 1 to 3 ring heteroatoms each independently selected from the group consisting of O, S, N, and NR^{d} and then are optionally substituted on a ring carbon with 1 to 3 groups each independently selected from the group consisting of halo, OH and CN;
Each R^{c} is independently selected from the group consisting of H, D, halo, OH, CN, C₁-C₄alkyl, and C₁-C₄alkoxy, or two R^{c}, attached to the same atom, form a ═O, wherein the C₁-C₄alkyl and C₁-C₄alkoxy are each optionally substituted with 1 to 3 groups each independently selected from the group consisting of halo, OH and CN;
Each R^{d} is independently selected from the group consisting of H, D, C₁-C₄alkyl, and C(O)C₁-4alkyl; and
n is 0, 1, 2, or 3.

### Twentieth Embodiment

a compound represented by Formula (VIa), (VIb), (VIc), (VId), (VIe), (VIf), (VIg), or (VIh):

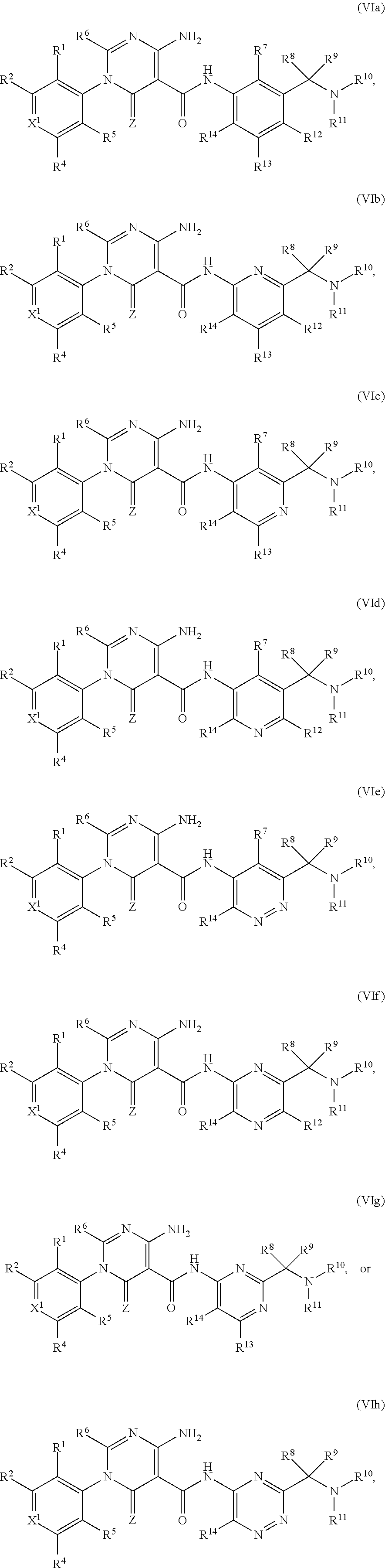

or a pharmaceutically acceptable salt thereof, wherein the definitions for the variables in Formulae (VIa), (VIb), (VIc), (VId), (VIe), (VIf), (VIg), and (VIh) are as defined in the first, eighteenth, or nineteenth embodiment.

### Twenty-First Embodiment

a compound represented by Formula (VII):

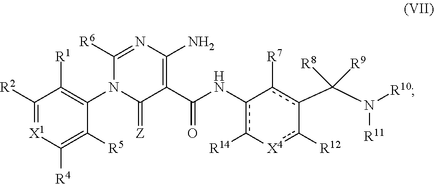

or a pharmaceutically acceptable salt thereof, wherein the definitions for the variables in Formula (VII) are as defined in the first, eighteenth, or nineteenth embodiment.

### Twenty-Second Embodiment

a compound represented by Formula (VIIa) or (VIIb):

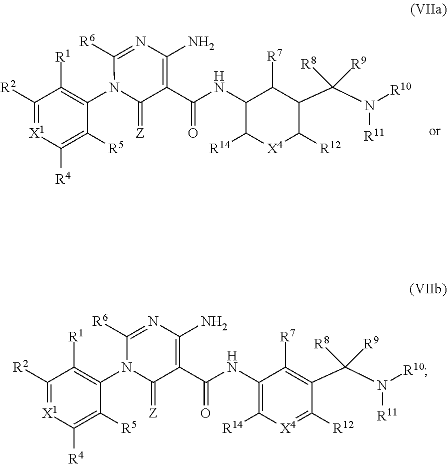

or a pharmaceutically acceptable salt thereof, wherein the definitions for the variables in Formula (VIIa) and Formula (VIIb) are as defined in the first, eighteenth, or nineteenth embodiment.

### Twenty-Third Embodiment

a compound represented by Formulae (I), (VIIa) or (VIIb), or a pharmaceutically acceptable salt thereof, wherein
X¹ is CR³ or N;
X⁴ is selected from the group consisting of CR¹³, N, and NR^{d};
R¹ is selected from the group consisting of halo, C₁-C₄alkyl, and C₁-C₄haloalkyl;
R² is selected from the group consisting of H, D, and C₁-C₄alkyl; R³ is selected from the group consisting of H, D, halo, CN, C₁-C₄alkyl, —(C(R^{a})₂)ₙ—OR^{b}, —(C(R^{a})₂)ₙ—C(O)OR^{b}, —(C(R^{a})₂)ₙ—SO₂—R^{b}, —N(R^{a})₂, C₃-C₈cycloalkyl, and 4 to 10-membered heterocyclyl, wherein the C₁-C₄alkyl and C₃-C₁₀cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are optionally substituted on a ring carbon with 1 to 3 R^{c};
R⁴ is selected from the group consisting of H, D, and C₁-C₄alkyl;
R⁵ is selected from the group consisting of halo, C₁-C₄alkyl, and C₁-C₄haloalkyl;
R⁶ is H or D;
R⁷ is selected from the group consisting of H, D, halo, C₁-C₄alkyl, and C₁-C₄alkoxy, C₃-C₉cycloalkyl, and 4 to 7-membered heterocyclyl, wherein the C₁-C₄alkyl, C₁-C₄alkoxy, and C₃-C₆cycloalkyl are each optionally substituted with 1 to 3 R^{c}, wherein the 4 to 7-membered heterocyclyl has 1 to 3 ring heteroatoms each independently selected from the group consisting of O, S, and NR^{d} and then are each optionally substituted on a ring carbon with 1 to 3 R^{c};
R⁸ is selected from the group consisting of H, D, and C₁-C₄alkyl;
R⁹ are each independently selected from the group consisting of H, D, C₁-C₄alkyl, C₂-C₄alkynyl, —(C(R^{a})₂)ₙ—OR^{b}, C₃-C₈cycloalkyl, and 4 to 10-membered heterocyclyl, wherein the C₁-C₄alkyl, C₂-C₄alkynyl, and C₃-C₈cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are optionally substituted on a ring carbon with 1 to 3 R^{c}; or
R⁸ and R⁹ are taken together with the carbon atom to which they are attached to form Ring B, wherein Ring B is C₃-C₈cycloalkyl or 4 to 10-membered heterocyclyl, wherein the C₃-C₈cycloalkyl is optionally substituted with 1 to 3 R^{c}, wherein the 4 to 10-membered heterocyclyl has 1 to 3 ring heteroatoms each independently selected from the group consisting of O, S, and NR^{d}, and then is optionally substituted on a ring carbon by 1 to 3 R^{c}; or
R⁸ and R⁹ are taken together form a ═O;
R¹⁰ is selected from the group consisting of H, D, C₁-C₄ alkyl, —(C(R^{a})₂)ₙ—OR^{b}, —(C(R^{a})₂)ₙ—SO₂R^{b}, C₃-C₈cycloalkyl, and 4 to 10-membered heterocyclyl, wherein the C₁-C₄ alkyl and C₃-C₈cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are optionally substituted on a ring carbon with 1 to 3 R^{c}; or
R⁹ and R¹⁰ are taken together with the carbon atom and the nitrogen atom to which they are attached, respectively, to form Ring C, wherein Ring C is a 4 to 10-membered heterocyclyl having 1 to 3 ring heteroatoms each independently selected from the group consisting of O, S, and NR^{d}, and then is optionally substituted on a ring carbon by 1 to 3 R^{c};
R¹¹ is selected from the group consisting of H, D, and C₁-C₄alkyl;
R¹² is selected from the group consisting of H, D, halo, and C₁-C₄alkyl;
R¹³ is selected from the group consisting of H, D, halo, CN, and C₁-C₄alkyl;
R¹⁴ is selected from the group consisting of H, D, and C₁-C₄alkyl;
Each R^{a} is independently selected from the group consisting of H, D, and C₁-C₄alkyl;
Each R^{b} is independently selected from the group consisting of H, D, C₁-C₄alkyl, C₁-C₄alkoxy, C₃-C₈cycloalkyl, and 4 to 10-membered heterocyclyl, wherein the C₁-C₄alkyl and C₃-C₈cycloalkyl are each optionally substituted with 1 to 3 groups each independently selected from the group consisting of halo, OH and CN, wherein the 4 to 10-membered heterocyclyl has 1 to 3 ring heteroatoms each independently selected from the group consisting of O, S, and NR^{d} and then are optionally substituted on a ring atom with 1 to 3 groups each independently selected from the group consisting of halo, OH and CN;
Each R^{c} is independently selected from the group consisting of D, halo, OH, CN, C₁-C₄alkyl, and C₁-C₄alkoxy, or two R^{a}, attached to the same atom, form a ═O, wherein the C₁-C₄alkyl and C₁-C₄alkoxy are each optionally substituted with 1 to 3 groups each independently selected from the group consisting of halo, OH and CN;
Each R^{d} is independently selected from the group consisting of H, D, C₁-C₄alkyl, and C(O)C₁-4alkyl; and
n is 0, 1, 2, or 3.

### Twenty-Fourth Embodiment

a compound represented by Formula (VIIIa), (VIIIb), (VIIIc), or (VIIId),

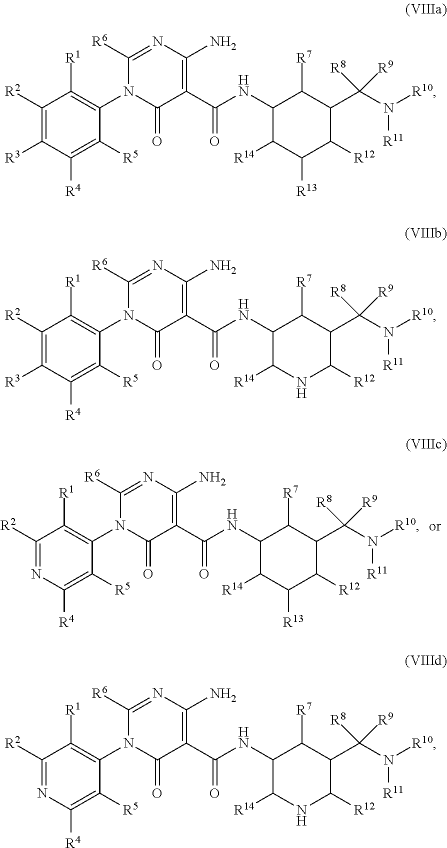

or a pharmaceutically acceptable salt thereof, wherein the definitions for the variables in Formulae (VIIIa), (VIIIb), (VIIIc) and (VIIId) are as defined in the first, eighteenth, nineteenth, or twenty-third embodiment.

### Twenty-Fifth Embodiment

a compound represented by Formula (VIIIe), (VIIIf), (VIIIg) or (VIIIh):

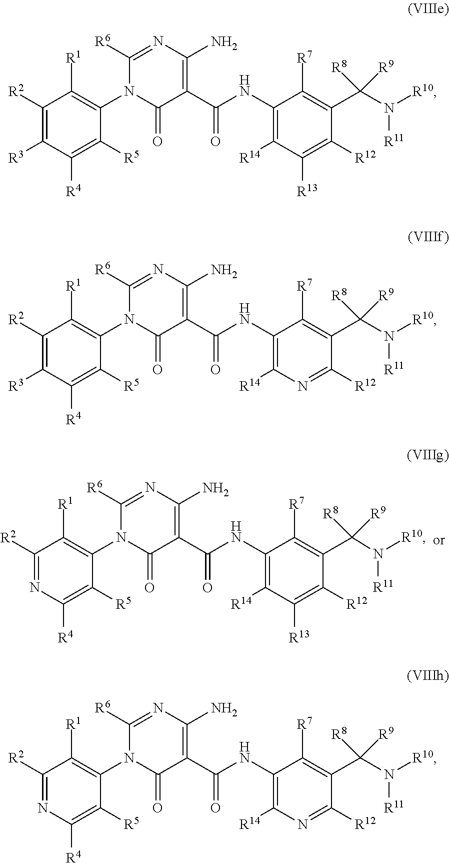

or a pharmaceutically acceptable salt thereof, wherein the definitions for the variables in Formulae (VIIIe), (VIIIf), (VIIIg) and (VIIIh) are as defined in the first, eighteenth, nineteenth, or twenty-third embodiment.

### Twenty-Sixth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹ is halo, —OH, C₁-C₄alkyl, or C₁-C₄alkoxy; and the remainder of the variables are as defined in the first aspect, or the first, eighteenth, nineteenth, or twenty-third embodiment.

### Twenty-Seventh Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹ is —CH₃, —C₁, —OH, or —OMe; and the remainder of the variables are as defined in the first aspect or the first, eighteenth, nineteenth, or twenty-third embodiment.

### Twenty-Eighth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh), and (X) or a pharmaceutically acceptable salt thereof, wherein R² is H, D, halo, or C₁-C₄alkyl; and the remainder of the variables are as defined in the first aspect or the first, eighteenth, nineteenth, twenty-third, twenty-sixth, or twenty-seventh embodiment.

### Twenty-Ninth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R² is H; and the remainder of the variables are as defined in the first aspect or the first, eighteenth, nineteenth, twenty-third, twenty-sixth, or twenty-seventh embodiment.

### Thirtieth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R³ is selected from the group consisting of H, D, halo, CN, C₁-C₄alkyl, —(C(R^{a})₂)ₙ—OR^{b}, —(C(R^{a})₂)ₙ—C(O)OR^{b}, —(C(R^{a})₂)ₙ—SO₂—R^{b}, —N(R^{a})₂, C₃-C₈cycloalkyl, 4 to 7-membered heterocyclyl, phenyl, and 4 to 6-membered heteroaryl, wherein the C₁-C₄alkyl, C₃-C₉cycloalkyl, and phenyl are each optionally substituted with 1 to 4 R^{c}, wherein the 4 to 7-membered heterocyclyl and 4 to 6-membered heteroaryl have 1 to 3 ring heteroatoms each independently selected from the group consisting of O, S, N, and NR^{d} and then are each optionally substituted on a ring carbon with 1 to 3 R^{c}; and the remainder of the variables are as defined in the first aspect or the first, eighteenth, nineteenth, twenty-third, twenty-sixth, twenty-seventh, twenty-eighth, or twenty-ninth embodiment.

### Thirty-First Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R³ is selected from the group consisting of —CH₂CH₃, —CH₂OCH₃, —CF₃, —OCH₃, H, —CN, —CHF₂, —CHFCH₃, —CHF—CH₂F, —CH(CH₃)OMe, —OCHF₂, C₁, —OCH₂CH₃, F, —CH₃, —O-cyclopropyl, —O—CH₂CH₂CH₃, —O—CF₃, —N(CH₃)₂, —O—CH(CH₃)₂, —CH₂OCH₂CH₃, —CH₂CHF₂, —CH₂CH₂F, —CH₂SO₂CH₃, —CF₂Me, —C(—O)OCH₃, —C(═O)OCH₂CH₂CH₃, —CH₂CF₃, and —CH₂CO₂H, or is represented by one of the following structures:

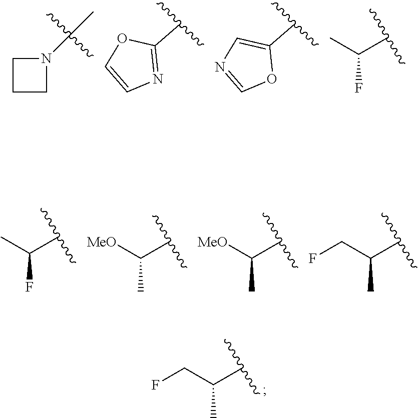

and the remainder of the variables are as defined in the first aspect or the first, eighteenth, nineteenth, twenty-third, twenty-sixth, twenty-seventh, twenty-eighth, or twenty-ninth embodiment.

### Thirty-Second Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R³ is —CH₂CH₃, —CH₂OCH₃, —CF₃, or —OCH₃; and the remainder of the variables are as defined in the first aspect or the first, eighteenth, nineteenth, twenty-third, twenty-sixth, twenty-seventh, twenty-eighth, or twenty-ninth embodiment.

### Thirty-Third Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁴ is H, D, halo, or C₁-C₄alkyl; and the remainder of the variables are as defined in the first aspect or the first, eighteenth, nineteenth, twenty-third, twenty-sixth, twenty-seventh, twenty-eighth, twenty-ninth, thirtieth, thirty-first, or thirty-second embodiment.

### Thirty-Fourth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁴ is H; and the remainder of the variables are as defined in the first aspect or the first, eighteenth, nineteenth, twenty-third, twenty-sixth, twenty-seventh, twenty-eighth, twenty-ninth, thirtieth, thirty-first, or thirty-second embodiment.

### Thirty-Fifth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁵ is halo, OH, C₁-C₄alkyl, C₁-C₄haloalkyl or C₁-C₄alkoxy; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, or thirty-fourth embodiment.

### Thirty-Sixth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁵ is Me, C₁, —OH, —CHF₂ or —OCH₃; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, or thirty-fourth embodiment.

### Thirty-Seventh Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁷ is H, D, halo, C₁-C₄alkyl, C₁-C₄alkoxy, or 4 to 7-membered heterocyclyl, wherein the C₁-C₄alkyl, and C₁-C₄alkoxy are each optionally substituted with 1 to 3 R^{c}, wherein the 4 to 7-membered heterocyclyl has 1 to 3 ring heteroatoms each independently selected from the group consisting of O, S, and NR^{d} and then are each optionally substituted on a ring carbon with 1 to 3 R^{c}; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, or thirty-sixth embodiment.

### Thirty-Eighth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁷ is H, morpholin-N-yl, —CF₃, —F, —OCHF₂, —OCH₂CH₃, or —OCH₃; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, or thirty-sixth embodiment.

### Thirty-Ninth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁷ is H; and the remainder of the variables are as defined in the first aspect or in the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, or thirty-sixth embodiments.

### Fortieth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁸ is H, D, halo, or C₁-C₄alkyl; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, or thirty-ninth embodiments.

### Forty-First Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁸ is H or —CH₃; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, or thirty-ninth embodiment.

### Forty-Second Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁸ is H; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, or thirty-ninth embodiment.

### Forty-Third Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁹ are each independently H, D, C₁-C₄alkyl, C₂-C₄alkynyl, —(C(R^{a})₂)ₙ—OR^{b}, or C₃-C₈cycloalkyl, wherein the C₁-C₄alkyl, C₂-C₄alkynyl, and C₃-C₈cycloalkyl are each optionally substituted with 1 to 3 R^{c}; and the remainder of the variables are as defined in the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, or forty-second embodiment.

### Forty-Fourth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁹ is H, Me, —CH₂OCH₂CH₃, —CH₂CH₂CH₃, —CH₂CH₃, —CH₂OCH₃, —CH₂CHF₂, cyclopropyl, —CH₂C═CH, —CH₂CH₂OCH₃, or —CHF₂; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, or forty-second embodiment.

### Forty-Fifth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R⁹ is H, Me, —CH₂OCH₂CH₃, —CH₂CH₂CH₃, —CH₂CH₃, or —CH₂OCH₃; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, or forty-second embodiment.

### Forty-Sixth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹⁰ is H, D, C₁-C₄ alkyl, —(C(R^{a})₂)ₙ—OR^{b}, —(C(R^{a})₂)ₙ—SO₂R^{b}, or C₃-C₈cycloalkyl, wherein the C₁-C₄ alkyl and C₃-C₈cycloalkyl are each optionally substituted with 1 to 3 R^{c}; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, or forty-fifth embodiment.

### Forty-Seventh Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹⁰ is selected from the group consisting of H, —CH₃, —CH₂CH₃, —CH₂CH₂CH₃, —CH₂CH₂SO₂CH₃, —CD₂CD₃, —CH₂CH₂OH, —CH₂CH₂OCH₃, —CH(CH₃)₂, or —CD₃; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, or forty-fifth embodiment.

### Forty-Eighth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹⁰ is H, —CH₃, or —CH₂CH₃; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, or forty-fifth embodiment.

### Forty-Ninth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹¹ is H, D, halo, or C₁-C₄alkyl; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, forty-fifth, forty-sixth, forty-seventh, or forty-eighth embodiment.

### Fiftieth Embodiment

a compound represented by any one of Formulae (I′), (I), (II), (IIa), (IIb), (IIc), (III), (Illa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹¹ is H; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, forty-fifth, forty-sixth, forty-seventh, or forty-eighth embodiment.

### Fifty-First Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹² is H, D, halo, or C₁-C₄alkyl; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, forty-fifth, forty-sixth, forty-seventh, forty-eighth, forty-ninth, or fiftieth embodiment.

### Fifty-Second Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹² is selected from the group consisting of H, D, or halo; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, forty-fifth, forty-sixth, forty-seventh, forty-eighth, forty-ninth, or fiftieth embodiment.

### Fifty-Third Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹² is H or F; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, forty-fifth, forty-sixth, forty-seventh, forty-eighth, forty-ninth, or fiftieth embodiment.

### Fifty-Fourth Embodiment

a compound represented by any one of Formulae (I′), (I), (II), (IIa), (IIb), (IIc), (III), (Illa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹² is H; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, forty-fifth, forty-sixth, forty-seventh, forty-eighth, forty-ninth, or fiftieth embodiment.

### Fifty-Fifth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹³ is H, D, halo, or C₁-C₄alkyl; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, forty-fifth, forty-sixth, forty-seventh, forty-eighth, forty-ninth, fiftieth, fifty-first, fifty-second, fifty-third or fifty-fourth embodiment.

### Fifty-Sixth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹³ is selected from the group consisting of H, D, or halo; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, forty-fifth, forty-sixth, forty-seventh, forty-eighth, forty-ninth, fiftieth, fifty-first, fifty-second, fifty-third or fifty-fourth embodiment.

### Fifty-Seventh Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹³ is H or F; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, forty-fifth, forty-sixth, forty-seventh, forty-eighth, forty-ninth, fiftieth, fifty-first, fifty-second, fifty-third or fifty-fourth embodiment.

### Fifty-Eighth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹³ is H; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, forty-fifth, forty-sixth, forty-seventh, forty-eighth, forty-ninth, fiftieth, fifty-first, fifty-second, or fifty-third embodiment.

### Fifty-Ninth Embodiment

a compound represented by any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh) and (X), or a pharmaceutically acceptable salt thereof, wherein R¹⁴ is H; and the remainder of the variables are as defined in the first aspect or the first, eighteenth, nineteenth, twenty-third, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, forty-fifth, forty-sixth, forty-seventh, forty-eighth, forty-ninth, fiftieth, fifty-first, fifty-second, fifty-third, fifty-fourth, fifty-fifth, fifty-sixth, fifty-seventh or fifty-eighth embodiment.

### Sixtieth Embodiment

a compound represented by Formula (IXa) or (IXb):

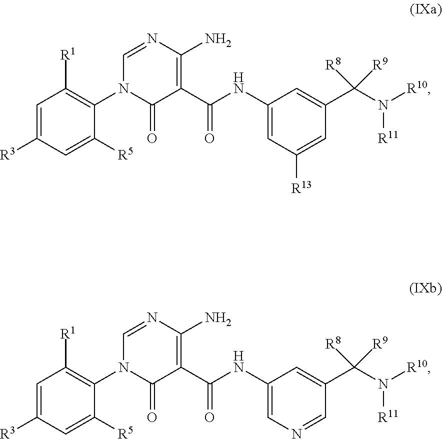

or a pharmaceutically acceptable salt thereof, wherein
R¹ is selected from the group consisting of halo, C₁-C₄alkyl, and C₁-C₄haloalkyl;
R³ is selected from the group consisting of H, D, halo, CN, C₁-C₄alkyl, —(C(R^{a})₂)ₙ—OR^{b}, —(C(R^{a})₂)ₙ—C(O)OR^{b}, —(C(R^{a})₂)ₙ—SO₂—R^{b}, —N(R^{a})₂, C₃-C₈cycloalkyl, and 4 to 10-membered heterocyclyl, wherein the C₁-C₄alkyl and C₃-C₈cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are optionally substituted on a ring carbon with 1 to 3 R^{c};
R⁵ is selected from the group consisting of halo, C₁-C₄alkyl, and C₁-C₄haloalkyl; R⁸ is selected from the group consisting of H, D, and C₁-C₄alkyl;
R⁹ is selected from the group consisting of H, D, C₁-C₄alkyl, —(C(R^{a})₂)ₙ—OR^{b}, C₃-C₈cycloalkyl, and 4 to 10-membered heterocyclyl, wherein the C₁-C₄alkyl and C₃-C₈cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are optionally substituted on a ring carbon with 1 to 3 R^{c};
R¹⁰ is selected from the group consisting of H, D, C₁-C₄ alkyl, C₃-C₁₀cycloalkyl, and 4 to 12-membered heterocyclyl, wherein the C₁-C₄ alkyl and C₃-C₁₀cycloalkyl are each optionally substituted with 1 to 4 R^{c}, wherein the 4 to 12-membered heterocyclyl has 1 to 4 ring heteroatoms each independently selected from the group consisting of O, S, and NR^{d} and then are optionally substituted on a ring carbon with 1 to 4 R^{c}; or
R⁹ and R¹⁰ are taken together with the carbon atom and the nitrogen atom to which they are attached, respectively, to form Ring C, wherein Ring C is a 4 to 10-membered heterocyclyl having 1 to 3 ring heteroatoms each independently selected from the group consisting of O, S, and NR^{d}, and then is optionally substituted on a ring carbon by 1 to 3 R^{c};
R¹¹ is selected from the group consisting of H, D, and C₁-C₄alkyl;
R¹³ is selected from the group consisting of H, D, halo, CN, and C₁-C₄alkyl; Each R^{a} is independently selected from the group consisting of H, D, and C₁-C₄alkyl;
Each R^{b} is independently selected from the group consisting of H, D, C₁-C₄alkyl, and C₁-C₄alkoxy;
Each R^{c} is independently selected from the group consisting of D, halo, OH, CN, C₁-C₄alkyl, and C₁-C₄alkoxy, or two R^{a}, attached to the same atom, form a ═O; and Each R^{d} is independently selected from the group consisting of H, D, and C₁-C₄alkyl; and
n is 0, 1, or 2.

### Sixty-First Embodiment

a compound represented by Formula (IXa) or (IXb), or a pharmaceutically acceptable salt thereof, wherein:
R¹ is halo or C₁-C₃alkyl;
R³ is C₁-C₃alkyl or —(C(R^{a})₂)ₙ—OR^{b}, wherein n is 0, and R^{b} is C₁-C₃alkyl, wherein the C₁-C₃alkyl is optionally substituted with 1 to 3 R^{c}, wherein R^{c} is independently halo or C₁-C₃alkoxy;
R⁵ is halo or C₁-C₃alkyl;
R⁸ is H;
R⁹ is C₁-C₃alkyl or —(C(R^{a})₂)ₙ—OR^{b}, wherein n is 0, and R^{b} is C₁-C₃alkyl, wherein the C₁-C₃alkyl is optionally substituted with 1 to 3 R^{c}, wherein R^{c} is independently halo or C₁-C₃alkoxy;
R¹⁰ is H or C₁-C₃alkyl; or
R⁹ and R¹⁰ are taken together with the carbon atom and the nitrogen atom to which they are attached, respectively, to form Ring C, wherein Ring C is a 4 to 6-membered heterocyclycl;
R¹¹ is H; and
R¹³ is H.

### Sixty-Second Embodiment

a compound represented by Formula (IXa) or (IXb), or a pharmaceutically acceptable salt thereof, wherein
R¹ is —CH₃ or —C₁;
R³ is —CH₂CH₃, —CH₂OCH₃, —CF₃ or —OCH₃;
R⁵ is —CH₃ or —C₁;
R⁸ is H;
R⁹ is H, —CH₃, —CH₂OCH₂CH₃, —CH₂CH₂CH₃, —CH₂CH₃, or —CH₂OCH₃;
R¹⁰ is H, —CH₃, or —CH₂CH₃; or
R⁹ and R¹⁰ are taken together with the carbon atom and the nitrogen atom to which they are attached, respectively, to form Ring C, wherein Ring C is represented by

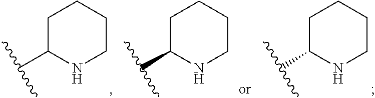

R¹¹ is H; and
R¹³ is H.

The disclosure also includes the compounds prepared in the Exemplification, in both the neutral form and pharmaceutically acceptable salts thereof. The synthetic protocol used to prepare the disclosed compounds is described in the Exemplification.

Another embodiment of the disclosure is a compound disclosed herein, including a compound of any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh), (IXa), (IXb), and (X), or a compound as disclosed in the Exemplification, or a pharmaceutically acceptable salt of any of the foregoing, in which one or more hydrogen atoms is replaced with deuterium. The deuterium enrichment at any one of the sites where hydrogen has been replaced by deuterium is at least 50%, 75%, 85%, 90%, 95%, 98% or 99%. Deuterium enrichment is a mole percent and is obtained by dividing the number of compounds with deuterium enrichment at the site of enrichment with the number of compounds having hydrogen or deuterium at the site of enrichment.

In some embodiments, the compound is selected from the table below, which shows structures of compounds described herein.

| | | LCMS | |
|---|---|---|---|
| Ex | Scheme | (MH+) | Structure |
| 1 | 1 | 458 | 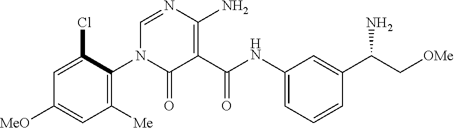 |
| 2 | 1 | 442 | 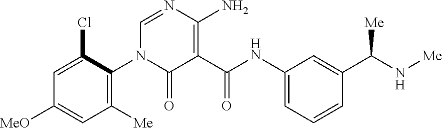 |
| 3 | 1 | 413 | 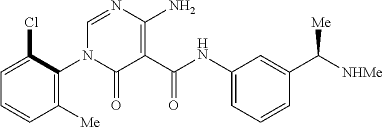 |
| 4 | 1 | 446 | 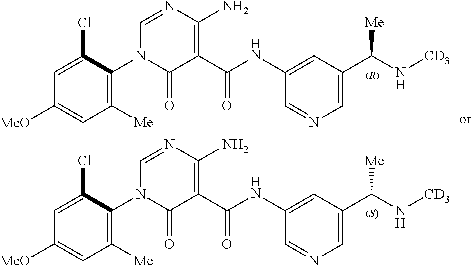 |
| 5 | 1 | 433 | 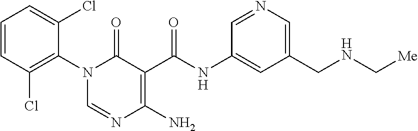 [SMILES: CCNCc1cncc(NC(=O)c2c(N)ncn(-c3c(Cl)cccc3Cl)c2=O)c1] |
| 6 | 1 | 450 | 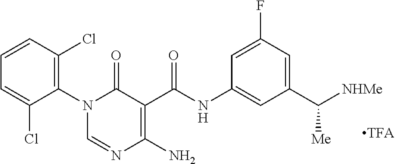 [SMILES: CN[C@H](C)c1cc(F)cc(NC(=O)c2c(N)ncn(-c3c(Cl)cccc3Cl)c2=O)c1] |
| 7 | 1 | 464 | 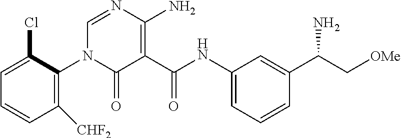 |
| 8 | 1 | 406 | 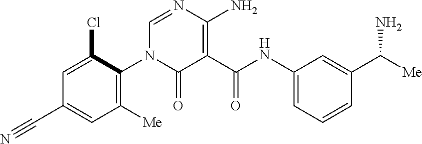 |
| 9 | 1 | 458 | 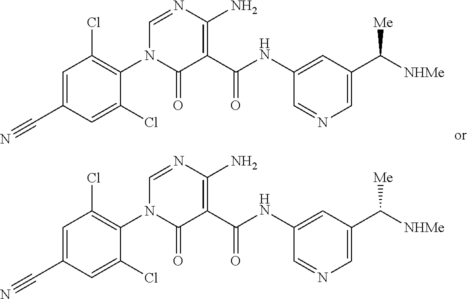 [SMILES: CN[C@H](C)c1cncc(NC(=O)c2c(N)ncn(-c3c(Cl)cc(C#N)cc3Cl)c2=O)c1 ; CN[C@@H](C)c1cncc(NC(=O)c2c(N)ncn(-c3c(Cl)cc(C#N)cc3Cl)c2=O)c1] |
| 10 | 1 | 468 | 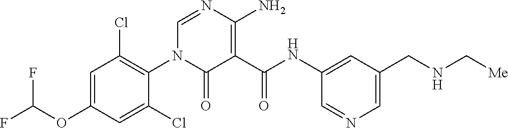 [SMILES: CCNCc1cncc(NC(=O)c2c(N)ncn(-c3c(Cl)cc(OC(F)F)cc3Cl)c2=O)c1] |
| 11 | 1 | 462 | 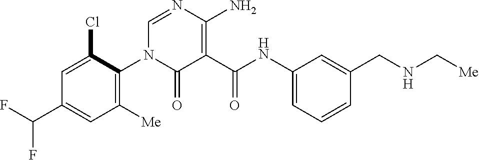 |
| 12 | 1 | 468 | 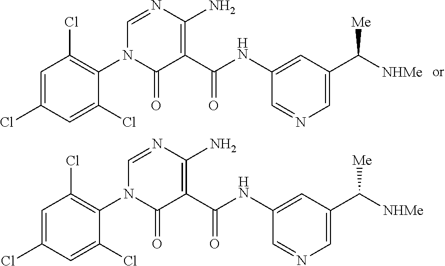 |
| 13 | 1 | 468 | 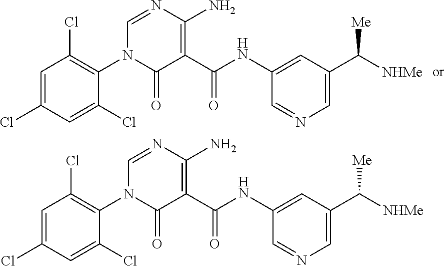 |
| 14 | 1 | 483 | 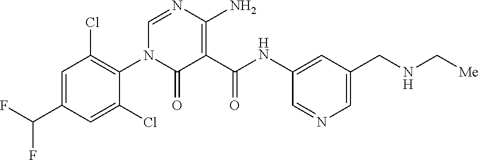 [SMILES: CCNCc1cncc(NC(=O)c2c(N)ncn(-c3c(Cl)cc(C(F)F)cc3Cl)c2=O)c1] |
| 15 | 1 | 476 | 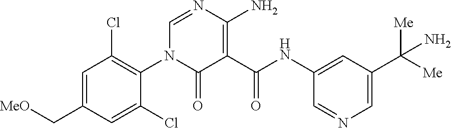 [SMILES: COCc1cc(Cl)c(-n2cnc(N)c(C(=O)Nc3cncc(C(C)(C)N)c3)c2=O)c(Cl)c1] |
| 16 | 1 | 484 | 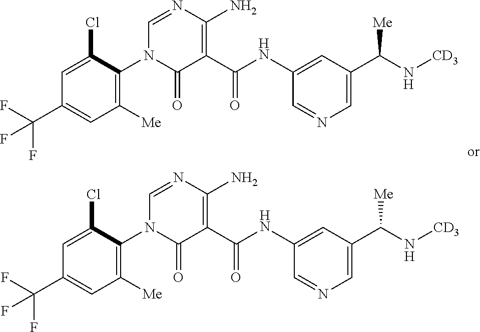 |
| 17 | 1 | 463 | 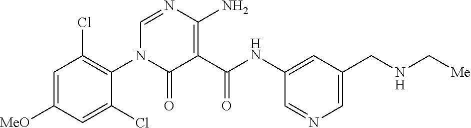 [SMILES: CCNCc1cncc(NC(=O)c2c(N)ncn(-c3c(Cl)cc(OC)cc3Cl)c2=O)c1] |
| 18 | 1 | 463 | 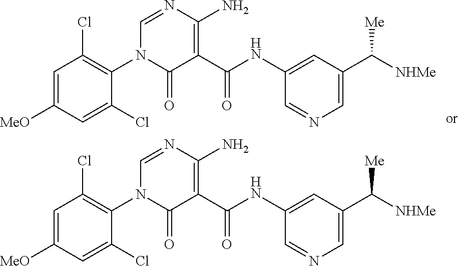 |
| 19 | 1 | 463 | 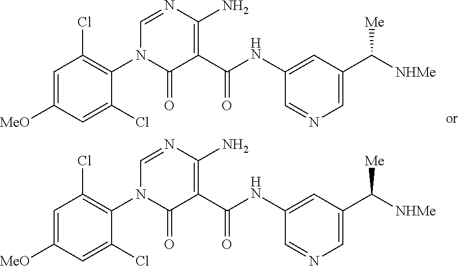 |
| 20 | 1 | 477 | 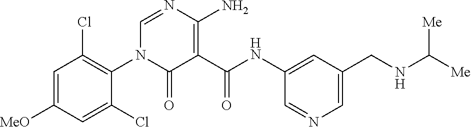 [SMILES: COc1cc(Cl)c(-n2cnc(N)c(C(=O)Nc3cncc(CNC(C)C)c3)c2=O)c(Cl)c1] |
| 21 | 1 | 489 | 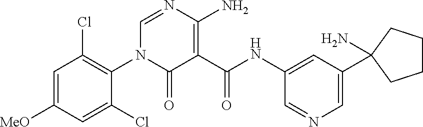 [SMILES: COc1cc(Cl)c(-n2cnc(N)c(C(=O)Nc3cncc(C4(N)CCCC4)c3)c2=O)c(Cl)c1] |
| 22 | 1 | 503 | 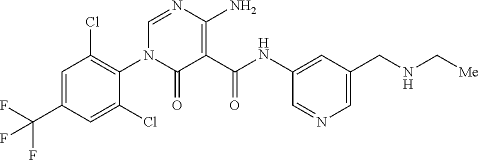 [SMILES: CCNCc1cncc(NC(=O)c2c(N)ncn(-c3c(Cl)cc(C(F)(F)F)cc3Cl)c2=O)c1] |
| 23 | 1 | 518 | 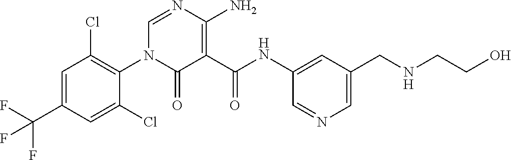 [SMILES: Nc1ncn(-c2c(Cl)cc(C(F)(F)F)cc2Cl)c(=O)c1C(=O)Nc1cncc(CNCCO)c1] |
| 24 | 1 | 477 | 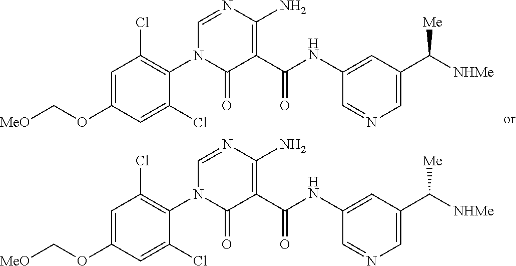 |
| 25 | 1 | 507 | 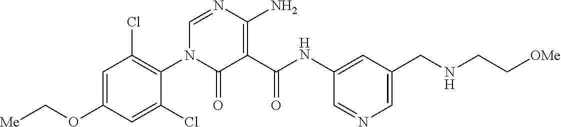 [SMILES: CCOc1cc(Cl)c(-n2cnc(N)c(C(=O)Nc3cncc(CNCCOC)c3)c2=O)c(Cl)c1] |
| 26 | 1 | 493 | 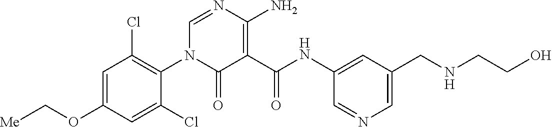 [SMILES: CCOc1cc(Cl)c(-n2cnc(N)c(C(=O)Nc3cncc(CNCCO)c3)c2=O)c(Cl)c1] |
| 27 | 1 | 449 | 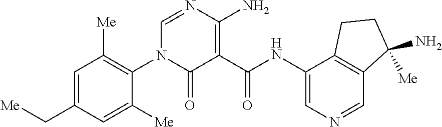 [SMILES: CCc1cc(C)c(-n2cnc(N)c(C(=O)Nc3cncc4c3CC[C@@]4(C)N)c2=O)c(C)c1] |
| | | | or |
| | | | 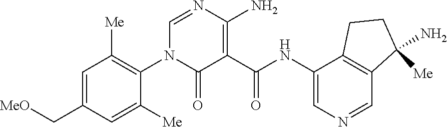 [SMILES: COCc1cc(C)c(-n2cnc(N)c(C(=O)Nc3cncc4c3CC[C@]4(C)N)c2=O)c(C)c1] |
| 28 | 1 | 464 | 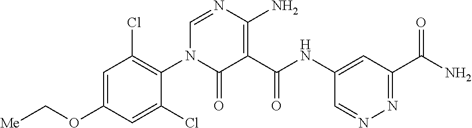 [SMILES: CCOc1cc(Cl)c(-n2cnc(N)c(C(=O)Nc3cnnc(C(N)=O)c3)c2=O)c(Cl)c1] |
| 29 | 1 | 424 | 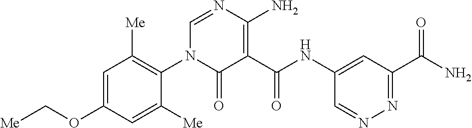 [SMILES: CCOc1cc(C)c(-n2cnc(N)c(C(=O)Nc3cnnc(C(N)=O)c3)c2=O)c(C)c1] |
| 30 | 1 | 424 | 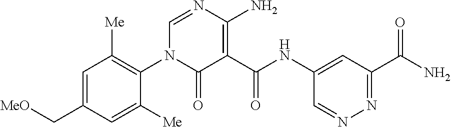 [SMILES: COCc1cc(C)c(-n2cnc(N)c(C(=O)Nc3cnnc(C(N)=O)c3)c2=O)c(C)c1] |
| 31 | 1 | 493 | 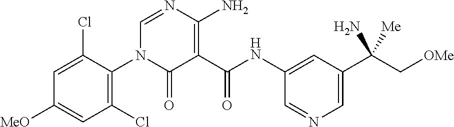 [SMILES: COC[C@](C)(N)c1cncc(NC(=O)c2c(N)ncn(-c3c(Cl)cc(OC)cc3Cl)c2=O)c1] |
| | | | or |
| | | | 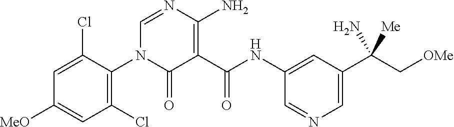 [SMILES: COC[C@@](C)(N)c1cncc(NC(=O)c2c(N)ncn(-c3c(Cl)cc(OC)cc3Cl)c2=O)c1] |
| 32 | 1 | 515 | 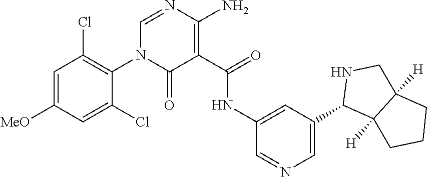 [SMILES: COc1cc(Cl)c(-n2cnc(N)c(C(=O)Nc3cncc([C@@H]4NC[C@H]5CCC[C@H]54)c3)c2=O)c(Cl)c1] |
| | | | or |
| | | | 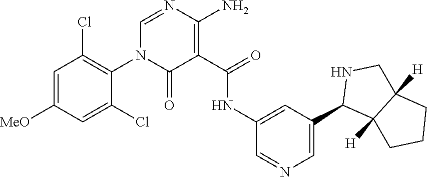 [SMILES: COc1cc(Cl)c(-n2cnc(N)c(C(=O)Nc3cncc([C@H]4NC[C@@H]5CCC[C@@H]54)c3)c2=O)c(Cl)c1] |
| 33 | 1 | 480 | 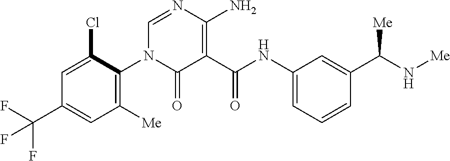 |
| 34 | 1 | 489 | 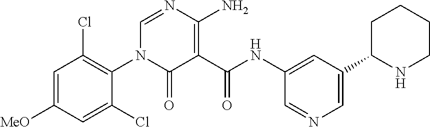 [SMILES: COc1cc(Cl)c(-n2cnc(N)c(C(=O)Nc3cncc([C@@H]4CCCCN4)c3)c2=O)c(Cl)c1] |
| 35 | 1 | 537 | 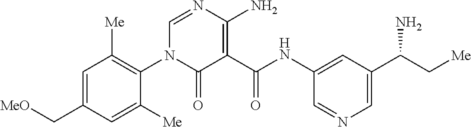 [SMILES: CC[C@@H](N)c1cncc(NC(=O)c2c(N)ncn(-c3c(C)cc(COC)cc3C)c2=O)c1] |
| | | | or |
| | | | 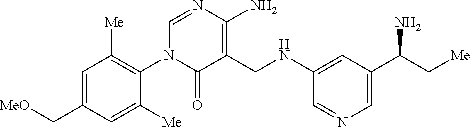 [SMILES: CC[C@H](N)c1cncc(NCc2c(N)ncn(-c3c(C)cc(COC)cc3C)c2=O)c1] |
| 36 | 1 | 454 | 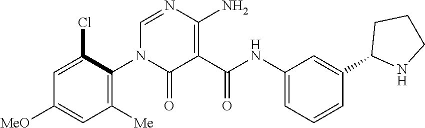 |
| 37 | 1 | 442 | 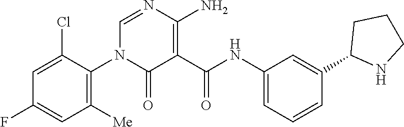 [SMILES: Cc1cc(F)cc(Cl)c1-n1cnc(N)c(C(=O)Nc2cccc([C@@H]3CCCN3)c2)c1=O] |
| 38 | 1 | 455 | 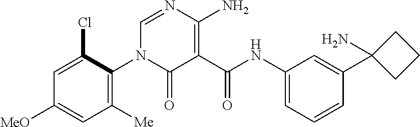 |
| 39 | 1 | 442 | 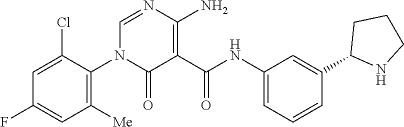 [SMILES: Cc1cc(F)cc(Cl)c1-n1cnc(N)c(C(=O)Nc2cccc([C@@H]3CCCN3)c2)c1=O] |
| 40 | 1 | 445 | 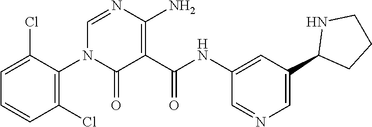 [SMILES: Nc1ncn(-c2c(Cl)cccc2Cl)c(=O)c1C(=O)Nc1cncc([C@@H]2CCCN2)c1] |
| 41 | 1 | 463 | 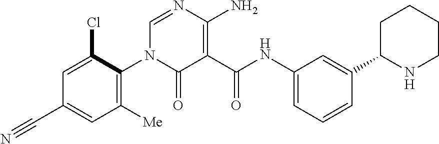 |
| 42 | 1 | 453 | 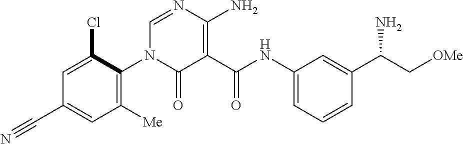 |
| 43 | | 444 | 1 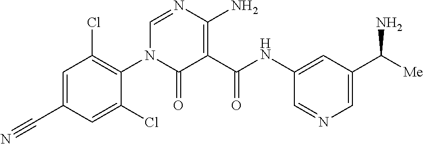 [SMILES: C[C@H](N)c1cncc(NC(=O)c2c(N)ncn(-c3c(Cl)cc(C#N)cc3Cl)c2=O)c1] |
| | | | or |
| | | | 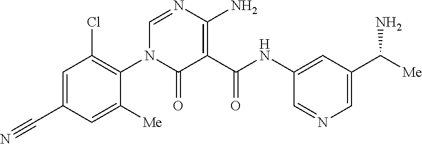 [SMILES: Cc1cc(C#N)cc(Cl)c1-n1cnc(N)c(C(=O)Nc2cncc([C@@H](C)N)c2)c1=O] |
| 44 | 1 | 474 | 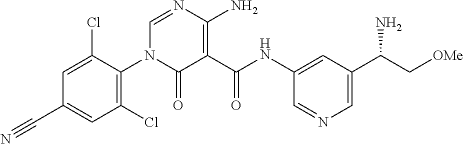 [SMILES: COC[C@@H](N)c1cncc(NC(=O)c2c(N)ncn(-c3c(Cl)cc(C#N)cc3Cl)c2=O)c1] |
| | | | or |
| | | | 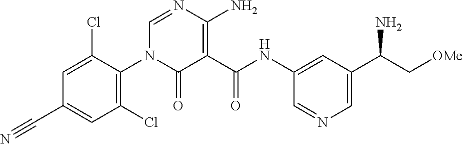 [SMILES: COC[C@H](N)c1cncc(NC(=O)c2c(N)ncn(-c3c(Cl)cc(C#N)cc3Cl)c2=O)c1] |
| 45 | 1 | 459 | 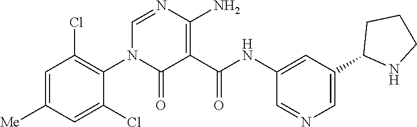 [SMILES: Cc1cc(Cl)c(-n2cnc(N)c(C(=O)Nc3cncc([C@@H]4CCCN4)c3)c2=O)c(Cl)c1] |
| 46 | 1 | 525 | 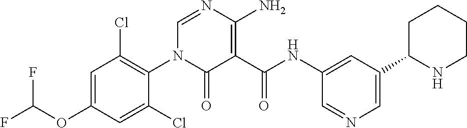 [SMILES: Nc1ncn(-c2c(Cl)cc(OC(F)F)cc2Cl)c(=O)c1C(=O)Nc1cncc([C@@H]2CCCCN2)c1] |
| 47 | 1 | 515 | 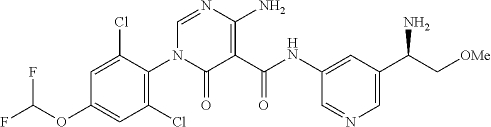 [SMILES: COC[C@H](N)c1cncc(NC(=O)c2c(N)ncn(-c3c(Cl)cc(OC(F)F)cc3Cl)c2=O)c1] |
| | | | or |
| | | | 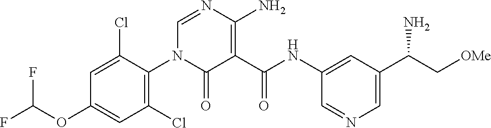 [SMILES: COC[C@@H](N)c1cncc(NC(=O)c2c(N)ncn(-c3c(Cl)cc(OC(F)F)cc3Cl)c2=O)c1] |
| 48 | 1 | 495 | 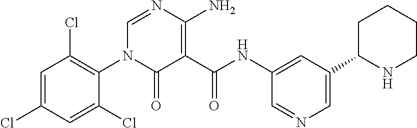 [SMILES: Nc1ncn(-c2c(Cl)cc(Cl)cc2Cl)c(=O)c1C(=O)Nc1cncc([C@@H]2CCCCN2)c1] |
| 49 | 1 | 484 | 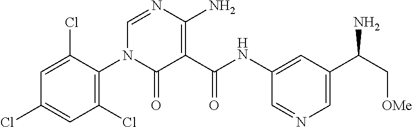 [SMILES: COC[C@H](N)c1cncc(NC(=O)c2c(N)ncn(-c3c(Cl)cc(Cl)cc3Cl)c2=O)c1] |
| | | | or |
| | | | 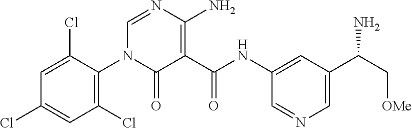 [SMILES: COC[C@@H](N)c1cncc(NC(=O)c2c(N)ncn(-c3c(Cl)cc(Cl)cc3Cl)c2=O)c1] |
| 50 | 1 | 453 | 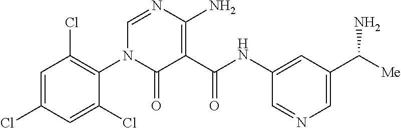 [SMILES: C[C@@H](N)c1cncc(NC(=O)c2c(N)ncn(-c3c(Cl)cc(Cl)cc3Cl)c2=O)c1] |
| | | | or |
| | | | 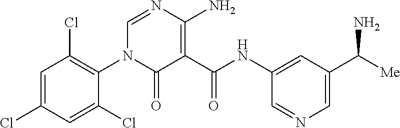 [SMILES: C[C@H](N)c1cncc(NC(=O)c2c(N)ncn(-c3c(Cl)cc(Cl)cc3Cl)c2=O)c1] |
| 51 | 1 | 475 | 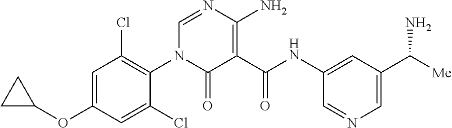 [SMILES: C[C@@H](N)c1cncc(NC(=O)c2c(N)ncn(-c3c(Cl)cc(OC4CC4)cc3Cl)c2=O)c1] |
| | | | or |
| | | | 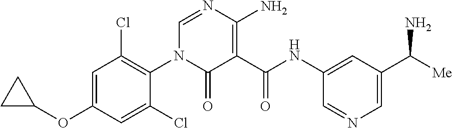 [SMILES: C[C@H](N)c1cncc(NC(=O)c2c(N)ncn(-c3c(Cl)cc(OC4CC4)cc3Cl)c2=O)c1] |
| 52 | 1 | 461 | 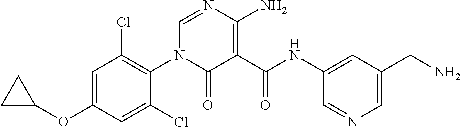 [SMILES: NCc1cncc(NC(=O)c2c(N)ncn(-c3c(Cl)cc(OC4CC4)cc3Cl)c2=O)c1] |
| 53 | 1 | 463 | 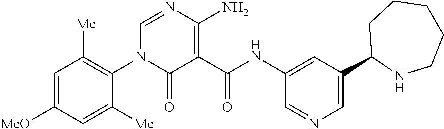 [SMILES: COc1cc(C)c(-n2cnc(N)c(C(=O)Nc3cncc([C@H]4CCCCCN4)c3)c2=O)c(C)c1] |
| | | | or |
| | | | 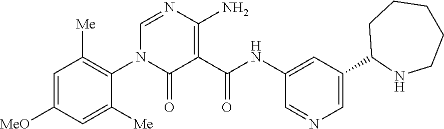 |
| 54 | 1 | 494 | 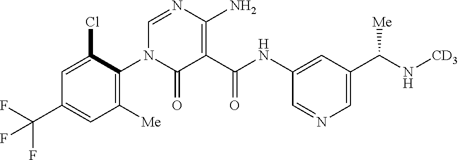 |
| 55 | 1 | 495 | 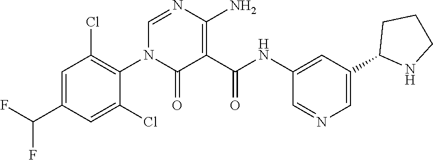 [SMILES: Nc1ncn(-c2c(Cl)cc(C(F)F)cc2Cl)c(=O)c1C(=O)Nc1cncc([C@@H]2CCCN2)c1] |
| 56 | 1 | 509 | 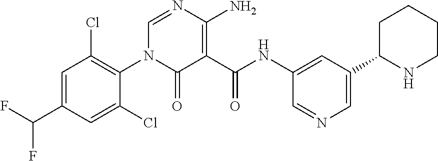 [SMILES: Nc1ncn(-c2c(Cl)cc(C(F)F)cc2Cl)c(=O)c1C(=O)Nc1cncc([C@@H]2CCCCN2)c1] |
| 57 | 1 | 469 | 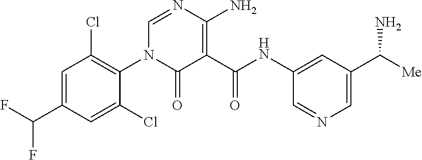 [SMILES: C[C@@H](N)c1cncc(NC(=O)c2c(N)ncn(-c3c(Cl)cc(C(F)F)cc3Cl)c2=O)c1] |
| | | | or |
| | | | 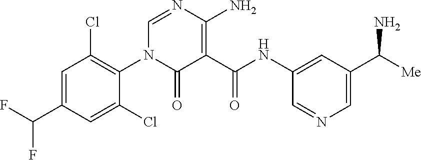 [SMILES: C[C@H](N)c1cncc(NC(=O)c2c(N)ncn(-c3c(Cl)cc(C(F)F)cc3Cl)c2=O)c1] |
| 58 | 1 | 499 | 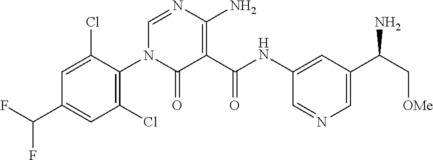 [SMILES: COC[C@H](N)c1cncc(NC(=O)c2c(N)ncn(-c3c(Cl)cc(C(F)F)cc3Cl)c2=O)c1] |
| | | | or |
| | | | 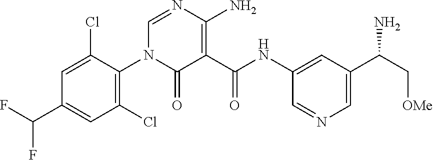 [SMILES: COC[C@@H](N)c1cncc(NC(=O)c2c(N)ncn(-c3c(Cl)cc(C(F)F)cc3Cl)c2=O)c1] |
| 59 | 1 | 525 | 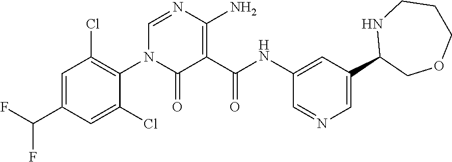 [SMILES: Nc1ncn(-c2c(Cl)cc(C(F)F)cc2Cl)c(=O)c1C(=O)Nc1cncc([C@@H]2COCCCN2)c1] |
| | | | or |
| | | | 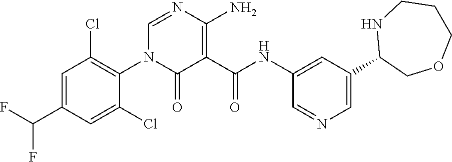 [SMILES: Nc1ncn(-c2c(Cl)cc(C(F)F)cc2Cl)c(=O)c1C(=O)Nc1cncc([C@H]2COCCCN2)c1] |
| 60 | 1 | 477 | 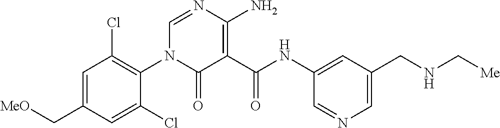 [SMILES: CCNCc1cncc(NC(=O)c2c(N)ncn(-c3c(Cl)cc(COC)cc3Cl)c2=O)c1] |
| 61 | 1 | 503 | 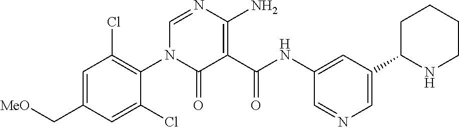 [SMILES: COCc1cc(Cl)c(-n2cnc(N)c(C(=O)Nc3cncc([C@@H]4CCCCN4)c3)c2=O)c(Cl)c1] |
| 62 | 1 | 493 | 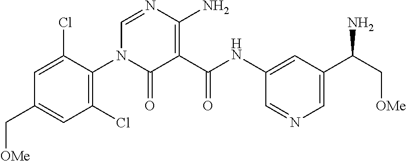 [SMILES: COCc1cc(Cl)c(-n2cnc(N)c(C(=O)Nc3cncc([C@@H](N)COC)c3)c2=O)c(Cl)c1] |
| | | | or |
| | | | 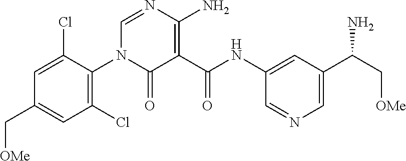 [SMILES: COCc1cc(Cl)c(-n2cnc(N)c(C(=O)Nc3cncc([C@H](N)COC)c3)c2=O)c(Cl)c1] |
| 63 | 1 | 493 | 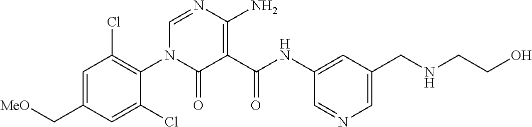 [SMILES: COCc1cc(Cl)c(-n2cnc(N)c(C(=O)Nc3cncc(CNCCO)c3)c2=O)c(Cl)c1] |
| 64 | 1 | 491 | 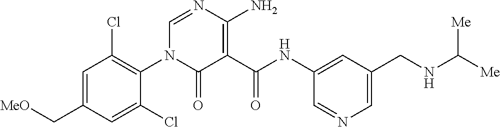 [SMILES: COCc1cc(Cl)c(-n2cnc(N)c(C(=O)Nc3cncc(CNC(C)C)c3)c2=O)c(Cl)c1] |
| 65 | 1 | 489 | 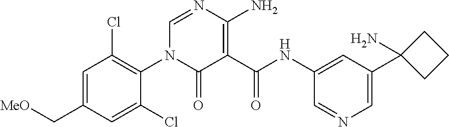 [SMILES: COCc1cc(Cl)c(-n2cnc(N)c(C(=O)Nc3cncc(C4(N)CCC4)c3)c2=O)c(Cl)c1] |
| 66 | 1 | 463 | 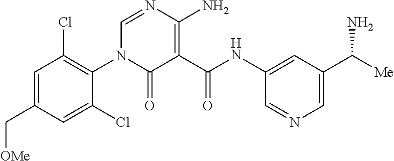 [SMILES: COCc1cc(Cl)c(-n2cnc(N)c(C(=O)Nc3cncc([C@@H](C)N)c3)c2=O)c(Cl)c1] |
| | | | or |
| | | | 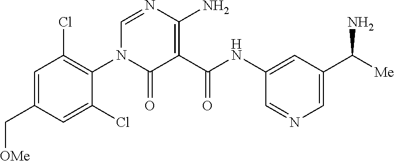 [SMILES: COCc1cc(Cl)c(-n2cnc(N)c(C(=O)Nc3cncc([C@H](C)N)c3)c2=O)c(Cl)c1] |
| 67 | 1 | 463 | 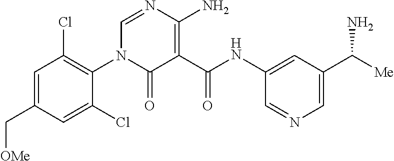 [SMILES: COCc1cc(Cl)c(-n2cnc(N)c(C(=O)Nc3cncc([C@@H](C)N)c3)c2=O)c(Cl)c1] |
| | | | or |
| | | | 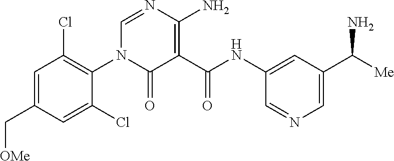 [SMILES: COCc1cc(Cl)c(-n2cnc(N)c(C(=O)Nc3cncc([C@H](C)N)c3)c2=O)c(Cl)c1] |
| 68 | 1 | 507 | 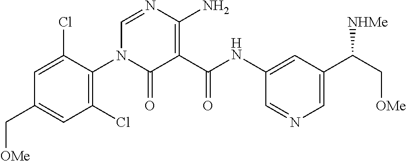 [SMILES: CN[C@H](COC)c1cncc(NC(=O)c2c(N)ncn(-c3c(Cl)cc(COC)cc3Cl)c2=O)c1] |
| | | | or |
| | | | 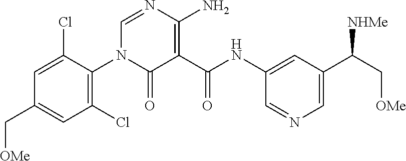 [SMILES: CN[C@@H](COC)c1cncc(NC(=O)c2c(N)ncn(-c3c(Cl)cc(COC)cc3Cl)c2=O)c1] |
| 69 | 1 | 507 | 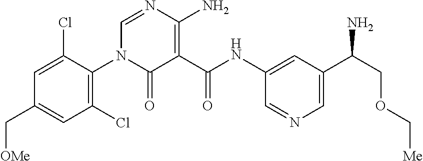 [SMILES: CCOC[C@H](N)c1cncc(NC(=O)c2c(N)ncn(-c3c(Cl)cc(COC)cc3Cl)c2=O)c1] |
| | | | or |
| | | | 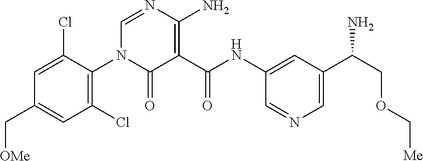 [SMILES: CCOC[C@@H](N)c1cncc(NC(=O)c2c(N)ncn(-c3c(Cl)cc(COC)cc3Cl)c2=O)c1] |
| 70 | 1 | 513 | 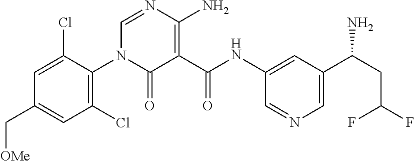 [SMILES: COCc1cc(Cl)c(-n2cnc(N)c(C(=O)Nc3cncc([C@H](N)CC(F)F)c3)c2=O)c(Cl)c1] |
| | | | or |
| | | | 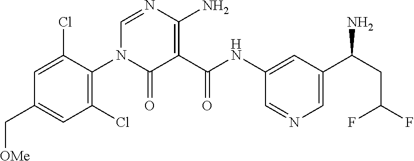 [SMILES: COCc1cc(Cl)c(-n2cnc(N)c(C(=O)Nc3cncc([C@@H](N)CC(F)F)c3)c2=O)c(Cl)c1] |
| 71 | 1 | 456 | 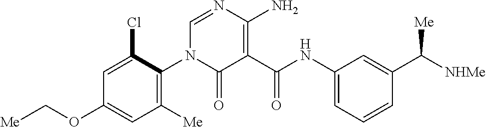 |
| 72 | 1 | 472 | 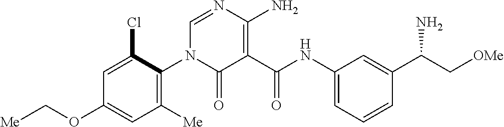 |
| 73 | 1 | 475 | 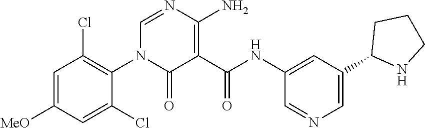 [SMILES: COc1cc(Cl)c(-n2cnc(N)c(C(=O)Nc3cncc([C@@H]4CCCN4)c3)c2=O)c(Cl)c1] |
| 74 | 1 | 449 | 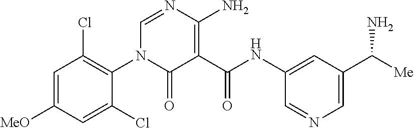 [SMILES: COc1cc(Cl)c(-n2cnc(N)c(C(=O)Nc3cncc([C@@H](C)N)c3)c2=O)c(Cl)c1] |
| | | | or |
| | | | 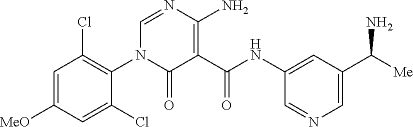 [SMILES: COc1cc(Cl)c(-n2cnc(N)c(C(=O)Nc3cncc([C@H](C)N)c3)c2=O)c(Cl)c1] |
| 75 | 1 | 505 | 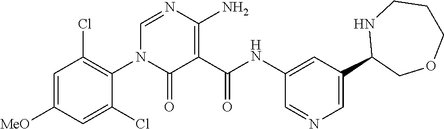 [SMILES: COc1cc(Cl)c(-n2cnc(N)c(C(=O)Nc3cncc([C@@H]4COCCCN4)c3)c2=O)c(Cl)c1] |
| | | | or |
| | | | 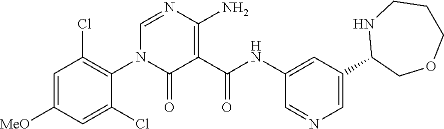 [SMILES: COc1cc(Cl)c(-n2cnc(N)c(C(=O)Nc3cncc([C@H]4COCCCN4)c3)c2=O)c(Cl)c1] |
| 76 | 1 | 507 | 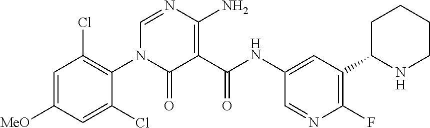 [SMILES: COc1cc(Cl)c(-n2cnc(N)c(C(=O)Nc3cnc(F)c([C@@H]4CCCCN4)c3)c2=O)c(Cl)c1] |
| | | | or |
| | | | 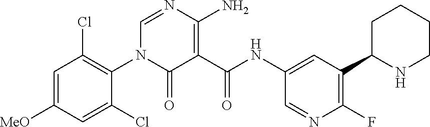 [SMILES: COc1cc(Cl)c(-n2cnc(N)c(C(=O)Nc3cnc(F)c([C@H]4CCCCN4)c3)c2=O)c(Cl)c1] |
| 77 | 1 | 479 | 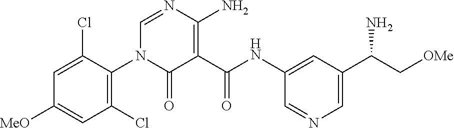 [SMILES: COC[C@@H](N)c1cncc(NC(=O)c2c(N)ncn(-c3c(Cl)cc(OC)cc3Cl)c2=O)c1] |
| 78 | 1 | 489 | 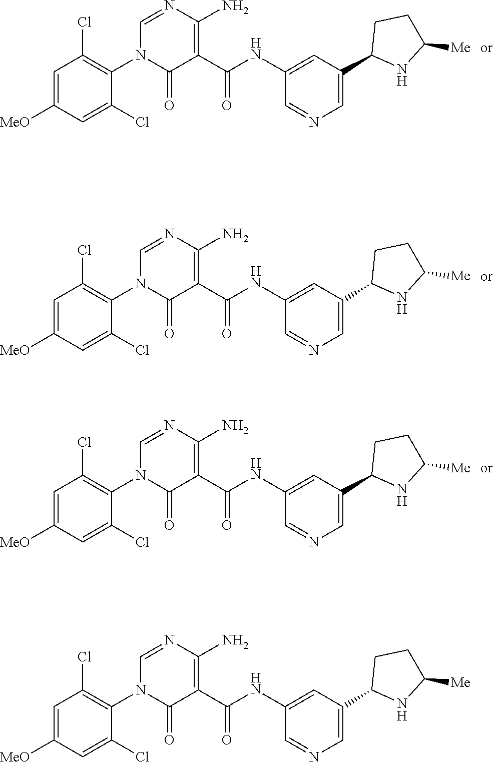 [SMILES: COc1cc(Cl)c(-n2cnc(N)c(C(=O)Nc3cncc([C@H]4CC[C@@H](C)N4)c3)c2=O)c(Cl)c1 ; COc1cc(Cl)c(-n2cnc(N)c(C(=O)Nc3cncc([C@@H]4CC[C@H](C)N4)c3)c2=O)c(Cl)c1 ; COc1cc(Cl)c(-n2cnc(N)c(C(=O)Nc3cncc([C@H]4CC[C@H](C)N4)c3)c2=O)c(Cl)c1 ; COc1cc(Cl)c(-n2cnc(N)c(C(=O)Nc3cncc([C@@H]4CC[C@@H](C)N4)c3)c2=O)c(Cl)c1] |
| 79 | 1 | 489 | 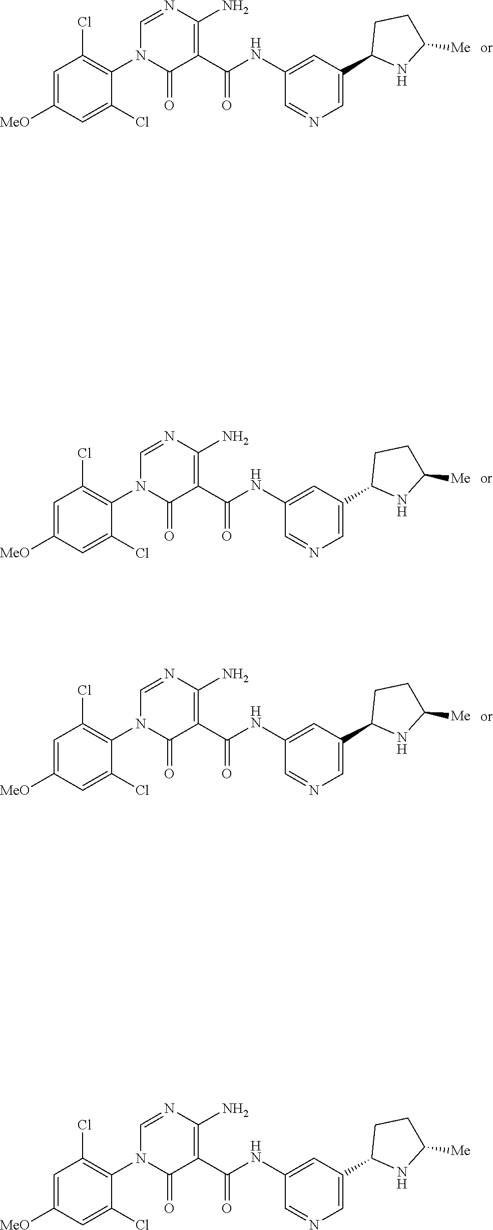 [SMILES: COc1cc(Cl)c(-n2cnc(N)c(C(=O)Nc3cncc([C@H]4CC[C@H](C)N4)c3)c2=O)c(Cl)c1 ; COc1cc(Cl)c(-n2cnc(N)c(C(=O)Nc3cncc([C@@H]4CC[C@@H](C)N4)c3)c2=O)c(Cl)c1 ; COc1cc(Cl)c(-n2cnc(N)c(C(=O)Nc3cncc([C@H]4CC[C@@H](C)N4)c3)c2=O)c(Cl)c1 ; COc1cc(Cl)c(-n2cnc(N)c(C(=O)Nc3cncc([C@@H]4CC[C@H](C)N4)c3)c2=O)c(Cl)c1] |
| 80 | 1 | 493 | 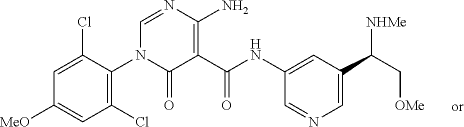 [SMILES: CN[C@@H](COC)c1cncc(NC(=O)c2c(N)ncn(-c3c(Cl)cc(OC)cc3Cl)c2=O)c1] |
| | | | 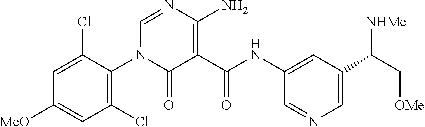 [SMILES: CN[C@H](COC)c1cncc(NC(=O)c2c(N)ncn(-c3c(Cl)cc(OC)cc3Cl)c2=O)c1] |
| 81 | 1 | 503 | 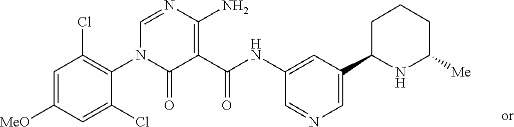 [SMILES: COc1cc(Cl)c(-n2cnc(N)c(C(=O)Nc3cncc([C@H]4CCC[C@H](C)N4)c3)c2=O)c(Cl)c1] |
| | | | 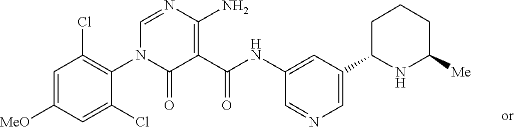 [SMILES: COc1cc(Cl)c(-n2cnc(N)c(C(=O)Nc3cncc([C@@H]4CCC[C@@H](C)N4)c3)c2=O)c(Cl)c1] |
| | | | 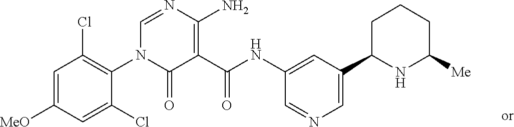 [SMILES: COc1cc(Cl)c(-n2cnc(N)c(C(=O)Nc3cncc([C@H]4CCC[C@@H](C)N4)c3)c2=O)c(Cl)c1] |
| | | | 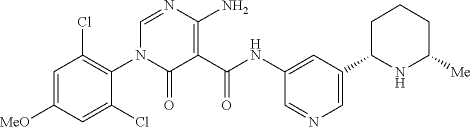 [SMILES: COc1cc(Cl)c(-n2cnc(N)c(C(=O)Nc3cncc([C@@H]4CCC[C@H](C)N4)c3)c2=O)c(Cl)c1] |
| 82 | 1 | 499 | 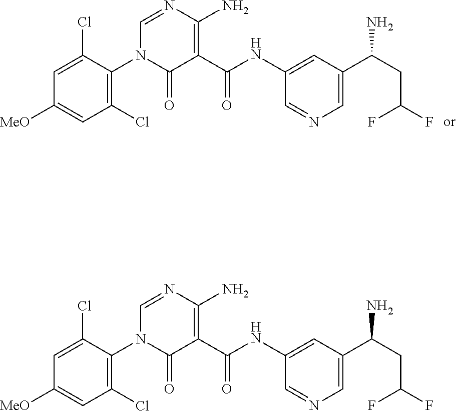 [SMILES: COc1cc(Cl)c(-n2cnc(N)c(C(=O)Nc3cncc([C@H](N)CC(F)F)c3)c2=O)c(Cl)c1 ; COc1cc(Cl)c(-n2cnc(N)c(C(=O)Nc3cncc([C@@H](N)CC(F)F)c3)c2=O)c(Cl)c1] |
| 83 | 1 | 503 | 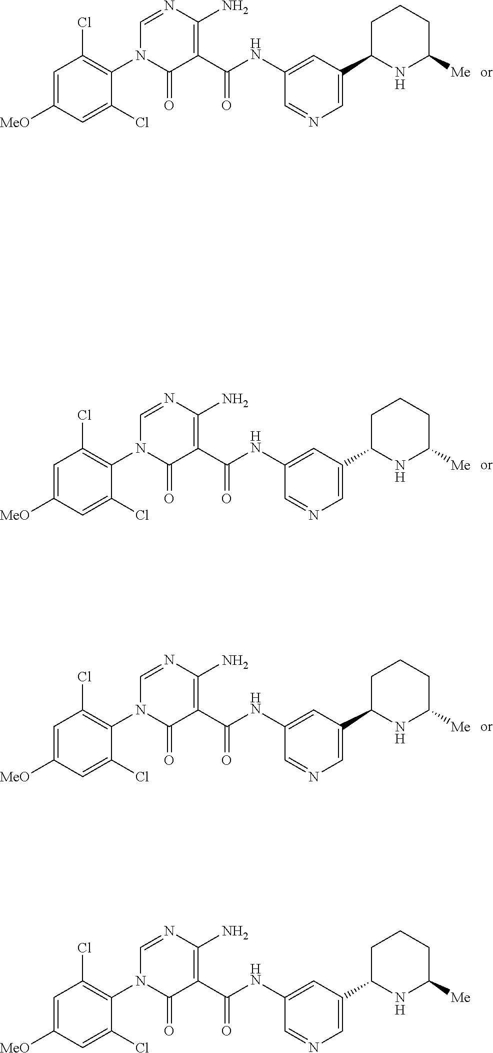 [SMILES: COc1cc(Cl)c(-n2cnc(N)c(C(=O)Nc3cncc([C@H]4CCC[C@@H](C)N4)c3)c2=O)c(Cl)c1 ; COc1cc(Cl)c(-n2cnc(N)c(C(=O)Nc3cncc([C@@H]4CCC[C@H](C)N4)c3)c2=O)c(Cl)c1 ; COc1cc(Cl)c(-n2cnc(N)c(C(=O)Nc3cncc([C@H]4CCC[C@H](C)N4)c3)c2=O)c(Cl)c1 ; COc1cc(Cl)c(-n2cnc(N)c(C(=O)Nc3cncc([C@@H]4CCC[C@@H](C)N4)c3)c2=O)c(Cl)c1] |
| 84 | 1 | 503 | 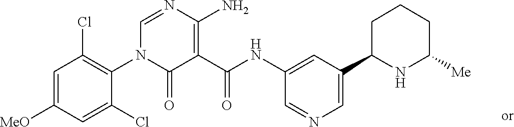 [SMILES: COc1cc(Cl)c(-n2cnc(N)c(C(=O)Nc3cncc([C@H]4CCC[C@H](C)N4)c3)c2=O)c(Cl)c1] |
| | | | 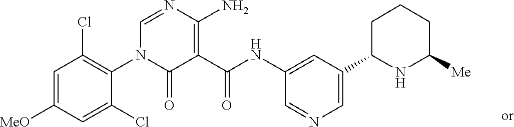 [SMILES: COc1cc(Cl)c(-n2cnc(N)c(C(=O)Nc3cncc([C@@H]4CCC[C@@H](C)N4)c3)c2=O)c(Cl)c1] |
| | | | 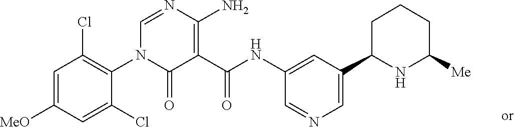 [SMILES: COc1cc(Cl)c(-n2cnc(N)c(C(=O)Nc3cncc([C@H]4CCC[C@@H](C)N4)c3)c2=O)c(Cl)c1] |
| | | | 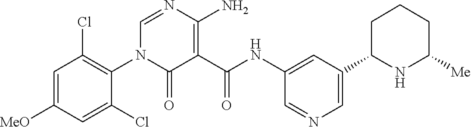 [SMILES: COc1cc(Cl)c(-n2cnc(N)c(C(=O)Nc3cncc([C@@H]4CCC[C@H](C)N4)c3)c2=O)c(Cl)c1] |
| 85 | 1 | 499 | 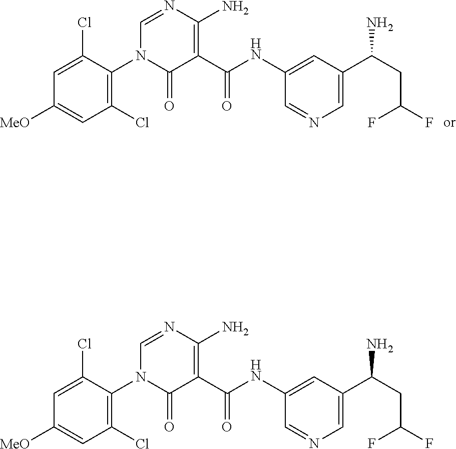 [SMILES: COc1cc(Cl)c(-n2cnc(N)c(C(=O)Nc3cncc([C@H](N)CC(F)F)c3)c2=O)c(Cl)c1 ; COc1cc(Cl)c(-n2cnc(N)c(C(=O)Nc3cncc([C@@H](N)CC(F)F)c3)c2=O)c(Cl)c1] |
| 86 | 1 | 491 | 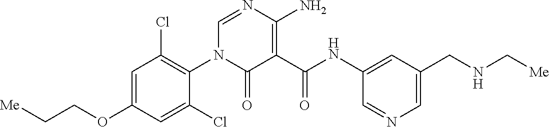 [SMILES: CCCOc1cc(Cl)c(-n2cnc(N)c(C(=O)Nc3cncc(CNCC)c3)c2=O)c(Cl)c1] |
| 87 | 1 | 487 | 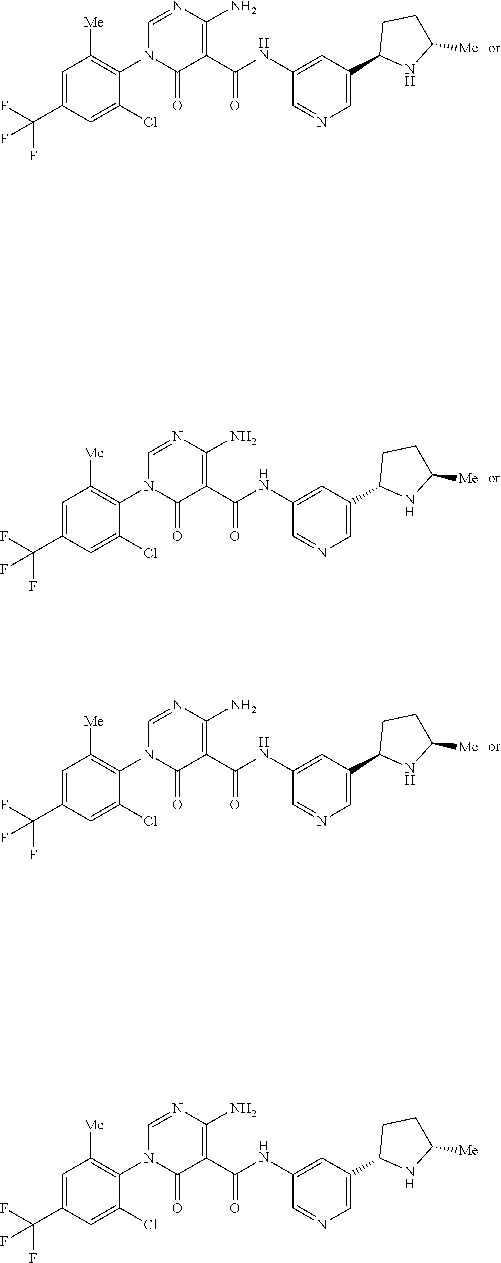 [SMILES: Cc1cc(C(F)(F)F)cc(Cl)c1-n1cnc(N)c(C(=O)Nc2cncc([C@H]3CC[C@H](C)N3)c2)c1=O ; Cc1cc(C(F)(F)F)cc(Cl)c1-n1cnc(N)c(C(=O)Nc2cncc([C@@H]3CC[C@@H](C)N3)c2)c1=O ; Cc1cc(C(F)(F)F)cc(Cl)c1-n1cnc(N)c(C(=O)Nc2cncc([C@H]3CC[C@@H](C)N3)c2)c1=O ; Cc1cc(C(F)(F)F)cc(Cl)c1-n1cnc(N)c(C(=O)Nc2cncc([C@@H]3CC[C@H](C)N3)c2)c1=O] |
| 88 | 1 | 461 | 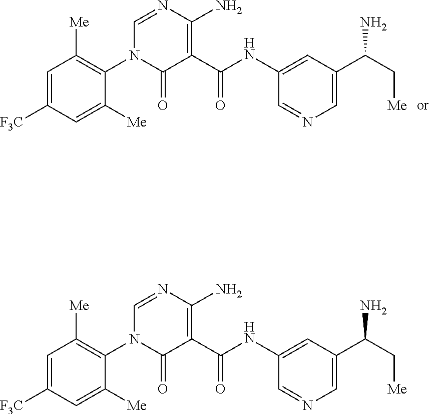 [SMILES: CC[C@@H](N)c1cncc(NC(=O)c2c(N)ncn(-c3c(C)cc(C(F)(F)F)cc3C)c2=O)c1 ; CC[C@H](N)c1cncc(NC(=O)c2c(N)ncn(-c3c(C)cc(C(F)(F)F)cc3C)c2=O)c1] |
| 89 | 1 | 501 | 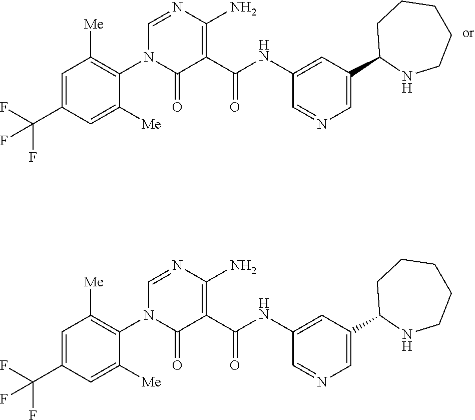 [SMILES: Cc1cc(C(F)(F)F)cc(C)c1-n1cnc(N)c(C(=O)Nc2cncc([C@H]3CCCCCN3)c2)c1=O ; Cc1cc(C(F)(F)F)cc(C)c1-n1cnc(N)c(C(=O)Nc2cncc([C@@H]3CCCCCN3)c2)c1=O] |
| 90 | 1 | 491 | 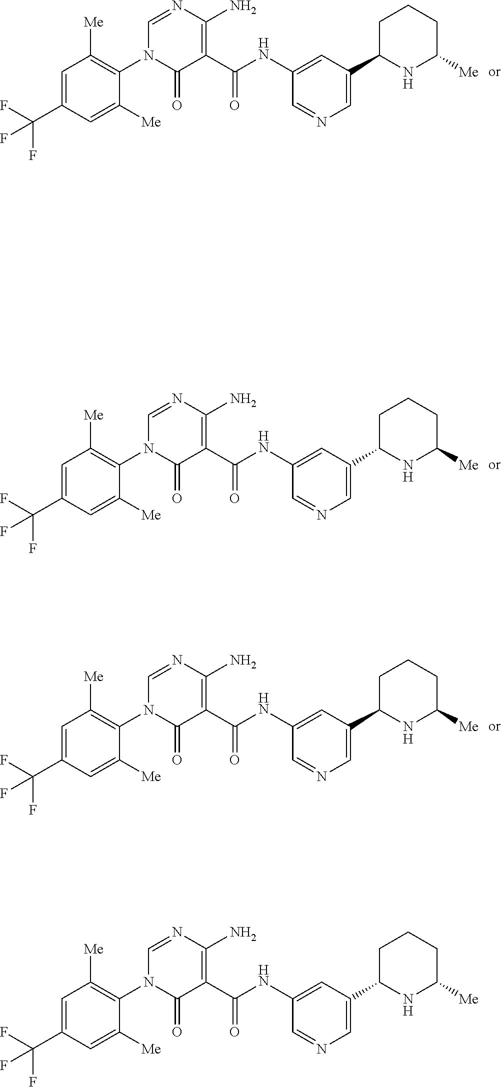 [SMILES: Cc1cc(C(F)(F)F)cc(C)c1-n1cnc(N)c(C(=O)Nc2cncc([C@H]3CCC[C@H](C)N3)c2)c1=O ; Cc1cc(C(F)(F)F)cc(C)c1-n1cnc(N)c(C(=O)Nc2cncc([C@@H]3CCC[C@@H](C)N3)c2)c1=O ; Cc1cc(C(F)(F)F)cc(C)c1-n1cnc(N)c(C(=O)Nc2cncc([C@H]3CCC[C@@H](C)N3)c2)c1=O ; Cc1cc(C(F)(F)F)cc(C)c1-n1cnc(N)c(C(=O)Nc2cncc([C@@H]3CCC[C@H](C)N3)c2)c1=O] |
| 91 | 1 | 463 | 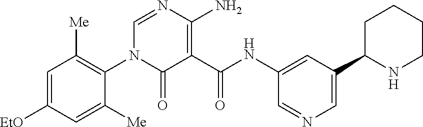 [SMILES: CCOc1cc(C)c(-n2cnc(N)c(C(=O)Nc3cncc([C@H]4CCCCN4)c3)c2=O)c(C)c1] |
| 92 | 1 | 463 | 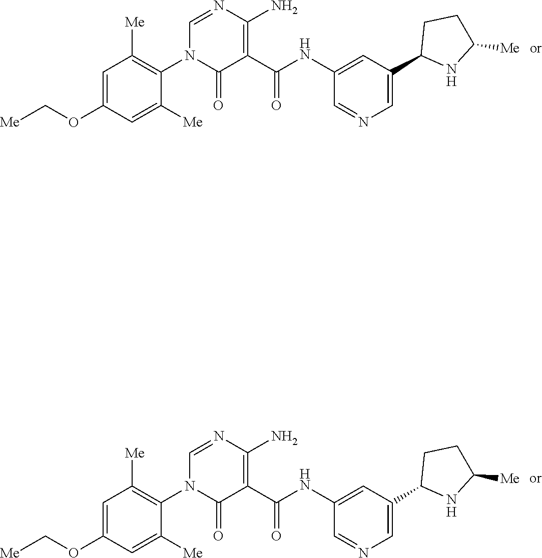 [SMILES: CCOc1cc(C)c(-n2cnc(N)c(C(=O)Nc3cncc([C@H]4CC[C@H](C)N4)c3)c2=O)c(C)c1 ; CCOc1cc(C)c(-n2cnc(N)c(C(=O)Nc3cncc([C@@H]4CC[C@@H](C)N4)c3)c2=O)c(C)c1] |
| 93 | 1 | 463 | 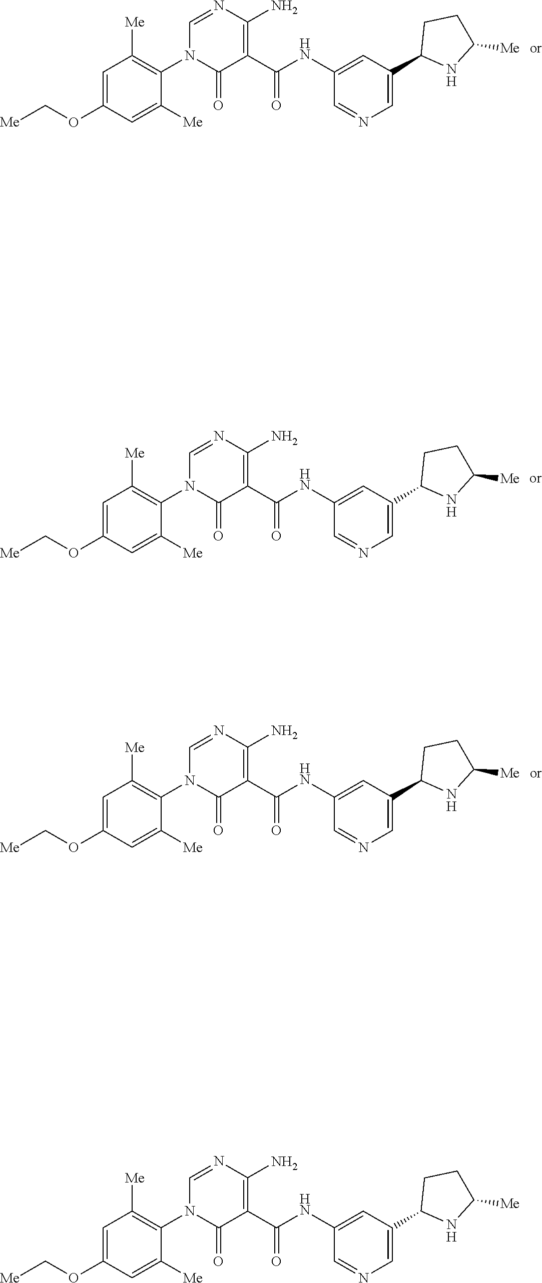 [SMILES: CCOc1cc(C)c(-n2cnc(N)c(C(=O)Nc3cncc([C@H]4CC[C@H](C)N4)c3)c2=O)c(C)c1 ; CCOc1cc(C)c(-n2cnc(N)c(C(=O)Nc3cncc([C@@H]4CC[C@@H](C)N4)c3)c2=O)c(C)c1 ; CCOc1cc(C)c(-n2cnc(N)c(C(=O)Nc3cncc([C@H]4CC[C@@H](C)N4)c3)c2=O)c(C)c1 ; CCOc1cc(C)c(-n2cnc(N)c(C(=O)Nc3cncc([C@@H]4CC[C@H](C)N4)c3)c2=O)c(C)c1] |
| 94 | 1 | 451 | 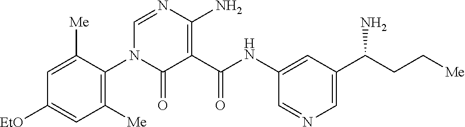 [SMILES: CCC[C@@H](N)c1cncc(NC(=O)c2c(N)ncn(-c3c(C)cc(OCC)cc3C)c2=O)c1] |
| 95 | 1 | 477 | 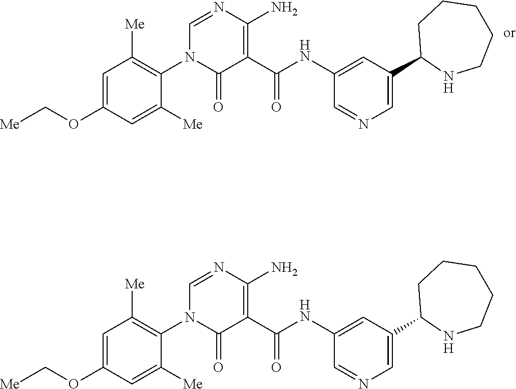 [SMILES: CCOc1cc(C)c(-n2cnc(N)c(C(=O)Nc3cncc([C@H]4CCCCCN4)c3)c2=O)c(C)c1 ; CCOc1cc(C)c(-n2cnc(N)c(C(=O)Nc3cncc([C@@H]4CCCCCN4)c3)c2=O)c(C)c1] |
| 96 | 1 | 477 | 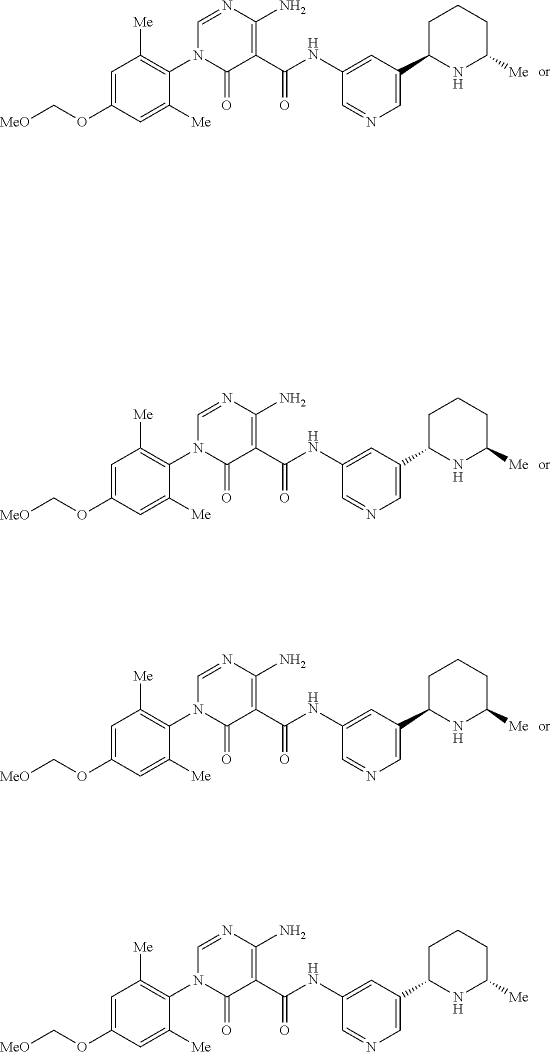 [SMILES: COCOc1cc(C)c(-n2cnc(N)c(C(=O)Nc3cncc([C@H]4CCC[C@H](C)N4)c3)c2=O)c(C)c1 ; COCOc1cc(C)c(-n2cnc(N)c(C(=O)Nc3cncc([C@@H]4CCC[C@@H](C)N4)c3)c2=O)c(C)c1 ; COCOc1cc(C)c(-n2cnc(N)c(C(=O)Nc3cncc([C@H]4CCC[C@@H](C)N4)c3)c2=O)c(C)c1 ; COCOc1cc(C)c(-n2cnc(N)c(C(=O)Nc3cncc([C@@H]4CCC[C@H](C)N4)c3)c2=O)c(C)c1] |
| 97 | 1 | 469 | 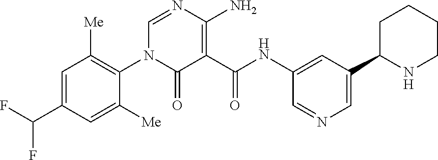 [SMILES: Cc1cc(C(F)F)cc(C)c1-n1cnc(N)c(C(=O)Nc2cncc([C@H]3CCCCN3)c2)c1=O] |
| 98 | 1 | 463 | 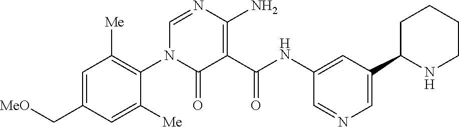 [SMILES: COCc1cc(C)c(-n2cnc(N)c(C(=O)Nc3cncc([C@H]4CCCCN4)c3)c2=O)c(C)c1] |
| 99 | 1 | 423 | 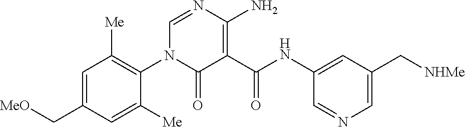 [SMILES: CNCc1cncc(NC(=O)c2c(N)ncn(-c3c(C)cc(COC)cc3C)c2=O)c1] |
| 100 | 1 | 477 | 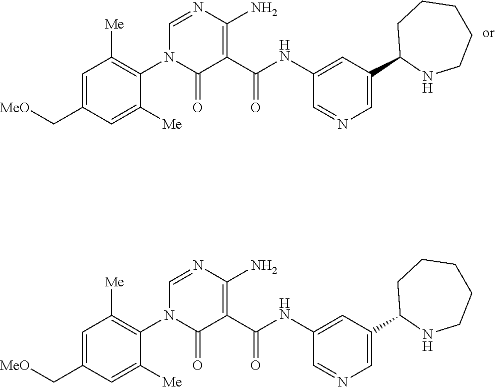 [SMILES: COCc1cc(C)c(-n2cnc(N)c(C(=O)Nc3cncc([C@H]4CCCCCN4)c3)c2=O)c(C)c1 ; COCc1cc(C)c(-n2cnc(N)c(C(=O)Nc3cncc([C@@H]4CCCCCN4)c3)c2=O)c(C)c1] |
| 101 | 1 | 463 | 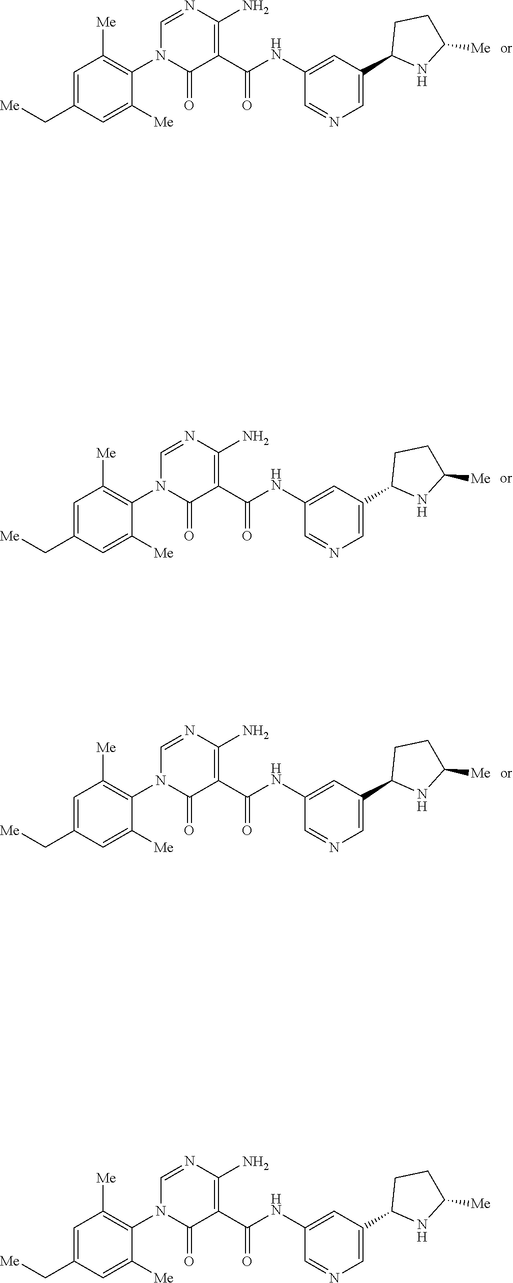 [SMILES: CCc1cc(C)c(-n2cnc(N)c(C(=O)Nc3cncc([C@H]4CC[C@H](C)N4)c3)c2=O)c(C)c1 ; CCc1cc(C)c(-n2cnc(N)c(C(=O)Nc3cncc([C@@H]4CC[C@@H](C)N4)c3)c2=O)c(C)c1 ; CCc1cc(C)c(-n2cnc(N)c(C(=O)Nc3cncc([C@H]4CC[C@@H](C)N4)c3)c2=O)c(C)c1 ; CCc1cc(C)c(-n2cnc(N)c(C(=O)Nc3cncc([C@@H]4CC[C@H](C)N4)c3)c2=O)c(C)c1] |
| 102 | 1 | 477 | 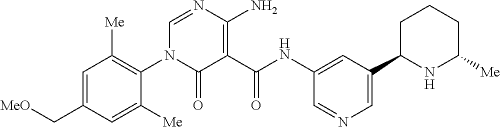 [SMILES: COCc1cc(C)c(-n2cnc(N)c(C(=O)Nc3cncc([C@H]4CCC[C@H](C)N4)c3)c2=O)c(C)c1] |
| | | | 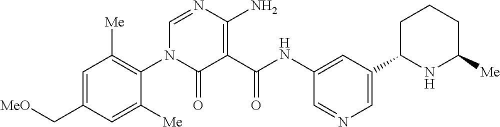 [SMILES: COCc1cc(C)c(-n2cnc(N)c(C(=O)Nc3cncc([C@@H]4CCC[C@@H](C)N4)c3)c2=O)c(C)c1] |
| | | | 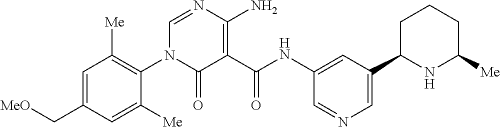 [SMILES: COCc1cc(C)c(-n2cnc(N)c(C(=O)Nc3cncc([C@H]4CCC[C@@H](C)N4)c3)c2=O)c(C)c1] |
| | | | 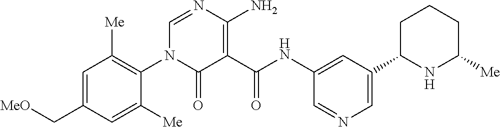 [SMILES: COCc1cc(C)c(-n2cnc(N)c(C(=O)Nc3cncc([C@@H]4CCC[C@H](C)N4)c3)c2=O)c(C)c1] |
| 103 | 1 | 463 | 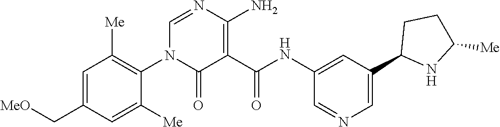 [SMILES: COCc1cc(C)c(-n2cnc(N)c(C(=O)Nc3cncc([C@H]4CC[C@H](C)N4)c3)c2=O)c(C)c1] |
| | | | 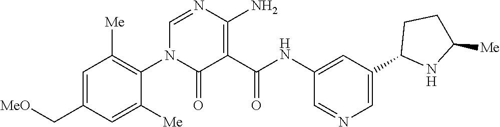 [SMILES: COCc1cc(C)c(-n2cnc(N)c(C(=O)Nc3cncc([C@@H]4CC[C@@H](C)N4)c3)c2=O)c(C)c1] |
| | | | 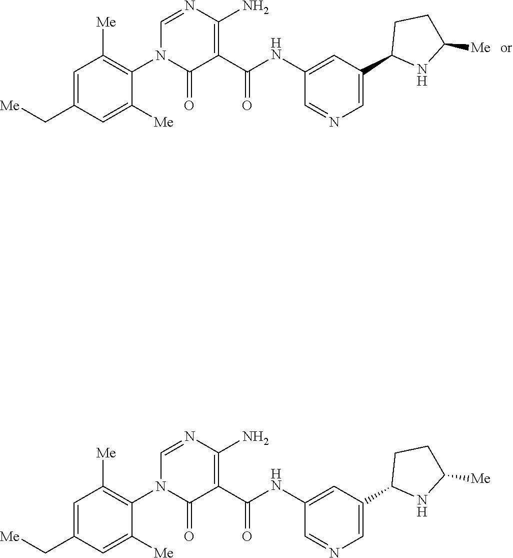 [SMILES: CCc1cc(C)c(-n2cnc(N)c(C(=O)Nc3cncc([C@H]4CC[C@@H](C)N4)c3)c2=O)c(C)c1 ; CCc1cc(C)c(-n2cnc(N)c(C(=O)Nc3cncc([C@@H]4CC[C@H](C)N4)c3)c2=O)c(C)c1] |
| 104 | 1 | 423 | 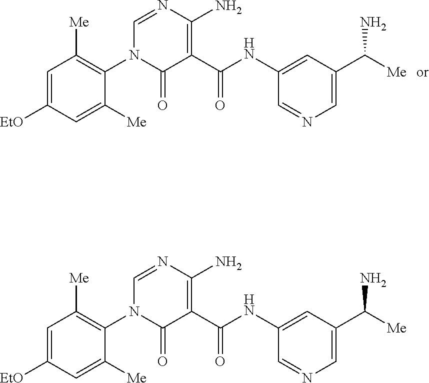 [SMILES: CCOc1cc(C)c(-n2cnc(N)c(C(=O)Nc3cncc([C@@H](C)N)c3)c2=O)c(C)c1 ; CCOc1cc(C)c(-n2cnc(N)c(C(=O)Nc3cncc([C@H](C)N)c3)c2=O)c(C)c1] |
| 105 | 1 | 429 | 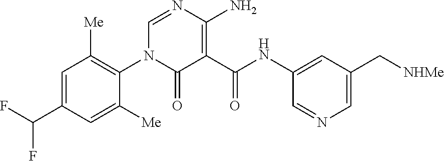 [SMILES: CNCc1cncc(NC(=O)c2c(N)ncn(-c3c(C)cc(C(F)F)cc3C)c2=O)c1] |
| 106 | 1 | 503 | 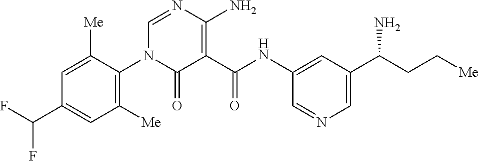 [SMILES: CCC[C@@H](N)c1cncc(NC(=O)c2c(N)ncn(-c3c(C)cc(C(F)F)cc3C)c2=O)c1] |
| 107 | 1 | 483 | 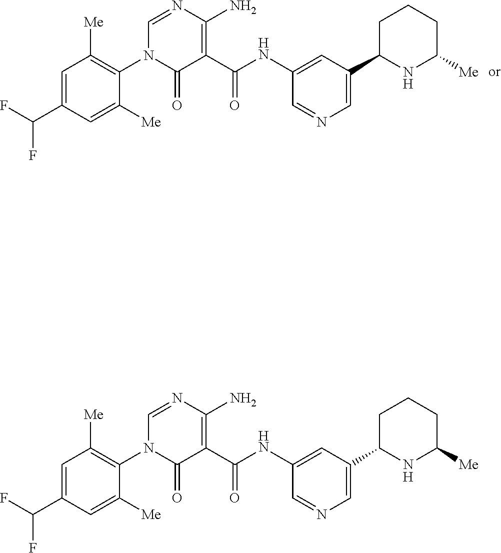 [SMILES: Cc1cc(C(F)F)cc(C)c1-n1cnc(N)c(C(=O)Nc2cncc([C@H]3CCC[C@H](C)N3)c2)c1=O ; Cc1cc(C(F)F)cc(C)c1-n1cnc(N)c(C(=O)Nc2cncc([C@@H]3CCC[C@@H](C)N3)c2)c1=O] |
| | | | 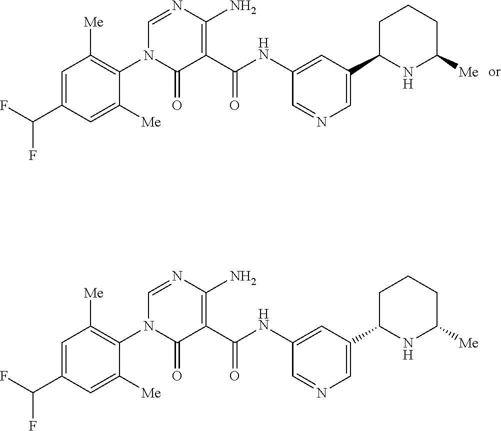 [SMILES: Cc1cc(C(F)F)cc(C)c1-n1cnc(N)c(C(=O)Nc2cncc([C@H]3CCC[C@@H](C)N3)c2)c1=O ; Cc1cc(C(F)F)cc(C)c1-n1cnc(N)c(C(=O)Nc2cncc([C@@H]3CCC[C@H](C)N3)c2)c1=O] |
| 108 | 1 | 483 | 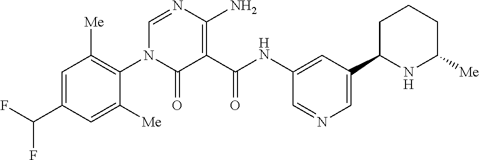 [SMILES: Cc1cc(C(F)F)cc(C)c1-n1cnc(N)c(C(=O)Nc2cncc([C@H]3CCC[C@H](C)N3)c2)c1=O] |
| | | | or |
| | | | 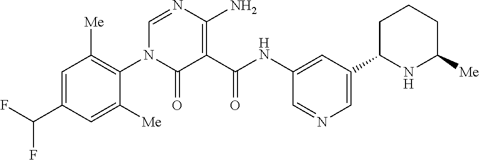 [SMILES: Cc1cc(C(F)F)cc(C)c1-n1cnc(N)c(C(=O)Nc2cncc([C@@H]3CCC[C@@H](C)N3)c2)c1=O] |
| | | | or |
| | | | 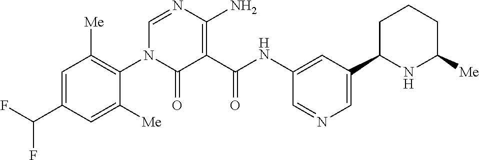 [SMILES: Cc1cc(C(F)F)cc(C)c1-n1cnc(N)c(C(=O)Nc2cncc([C@H]3CCC[C@@H](C)N3)c2)c1=O] |
| | | | or |
| | | | 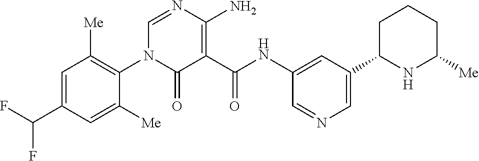 [SMILES: Cc1cc(C(F)F)cc(C)c1-n1cnc(N)c(C(=O)Nc2cncc([C@@H]3CCC[C@H](C)N3)c2)c1=O] |
| 109 | 1 | 529 | 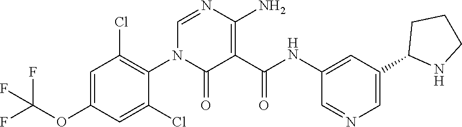 [SMILES: Nc1ncn(-c2c(Cl)cc(OC(F)(F)F)cc2Cl)c(=O)c1C(=O)Nc1cncc([C@@H]2CCCN2)c1] |
| 110 | 1 | 503 | 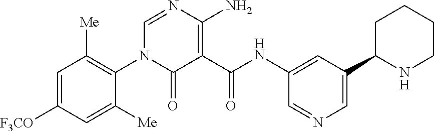 [SMILES: Cc1cc(OC(F)(F)F)cc(C)c1-n1cnc(N)c(C(=O)Nc2cncc([C@H]3CCCCN3)c2)c1=O] |
| 111 | 1 | 483 | 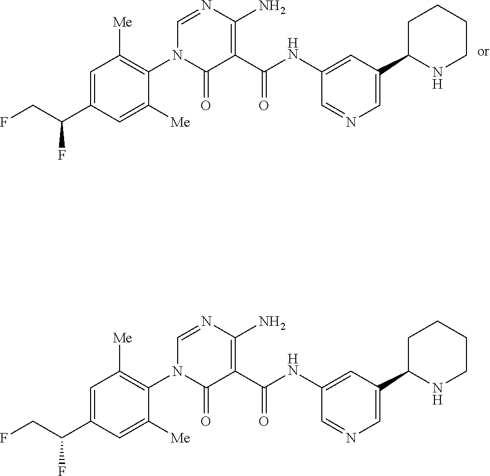 [SMILES: Cc1cc([C@@H](F)CF)cc(C)c1-n1cnc(N)c(C(=O)Nc2cncc([C@H]3CCCCN3)c2)c1=O ; Cc1cc([C@H](F)CF)cc(C)c1-n1cnc(N)c(C(=O)Nc2cncc([C@H]3CCCCN3)c2)c1=O] |
| 112 | 1 | 497 | 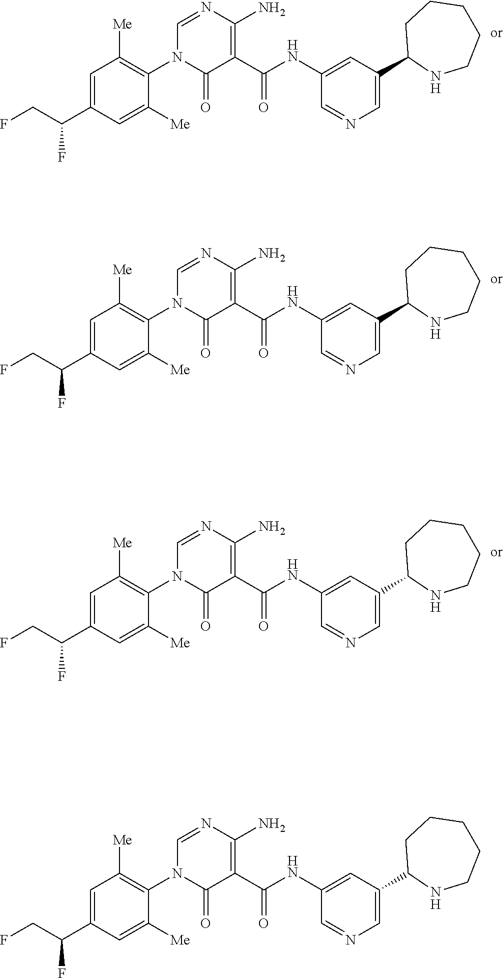 |
| 113 | 1 | 483 | 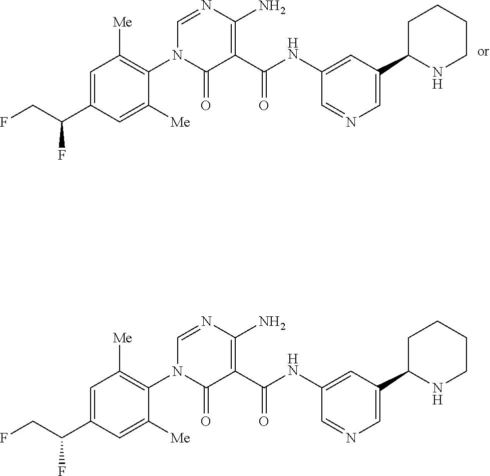 [SMILES: Cc1cc([C@@H](F)CF)cc(C)c1-n1cnc(N)c(C(=O)Nc2cncc([C@H]3CCCCN3)c2)c1=O ; Cc1cc([C@H](F)CF)cc(C)c1-n1cnc(N)c(C(=O)Nc2cncc([C@H]3CCCCN3)c2)c1=O] |
| 114 | 1 | 443 | 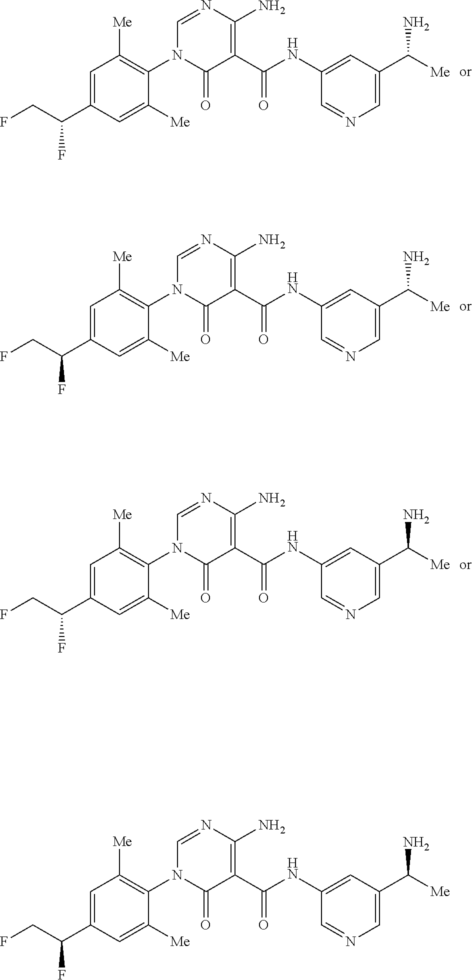 [SMILES: Cc1cc([C@H](F)CF)cc(C)c1-n1cnc(N)c(C(=O)Nc2cncc([C@@H](C)N)c2)c1=O ; Cc1cc([C@@H](F)CF)cc(C)c1-n1cnc(N)c(C(=O)Nc2cncc([C@@H](C)N)c2)c1=O ; Cc1cc([C@H](F)CF)cc(C)c1-n1cnc(N)c(C(=O)Nc2cncc([C@H](C)N)c2)c1=O ; Cc1cc([C@@H](F)CF)cc(C)c1-n1cnc(N)c(C(=O)Nc2cncc([C@H](C)N)c2)c1=O] |
| 115 | 1 | 497 | 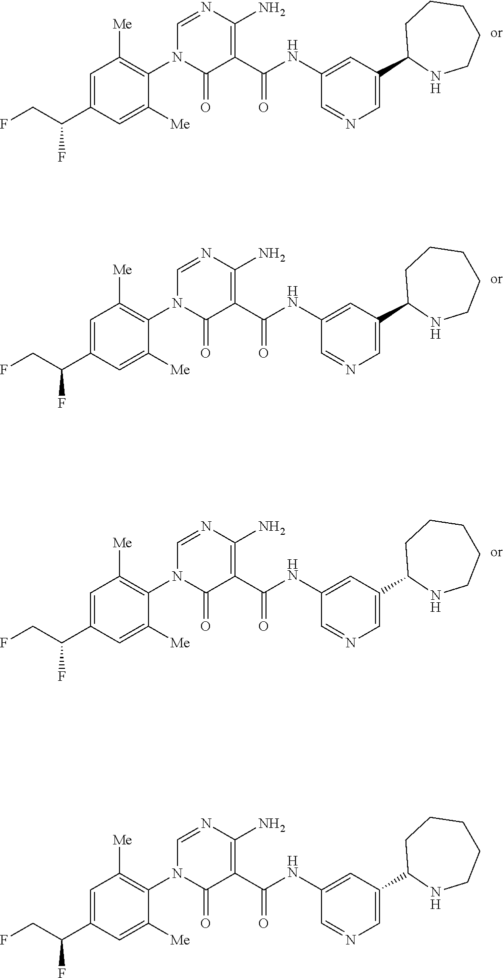 |
| 116 | 1 | 483 | 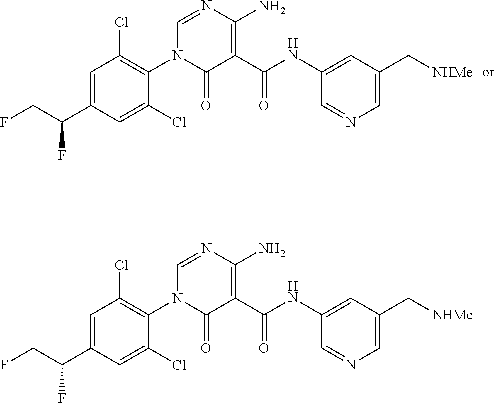 [SMILES: CNCc1cncc(NC(=O)c2c(N)ncn(-c3c(Cl)cc([C@@H](F)CF)cc3Cl)c2=O)c1 ; CNCc1cncc(NC(=O)c2c(N)ncn(-c3c(Cl)cc([C@H](F)CF)cc3Cl)c2=O)c1] |
| 117 | 1 | 483 | 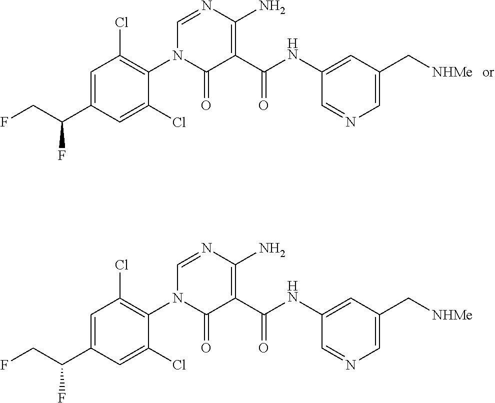 [SMILES: CNCc1cncc(NC(=O)c2c(N)ncn(-c3c(Cl)cc([C@@H](F)CF)cc3Cl)c2=O)c1 ; CNCc1cncc(NC(=O)c2c(N)ncn(-c3c(Cl)cc([C@H](F)CF)cc3Cl)c2=O)c1] |
| 118 | 1 | 514 | 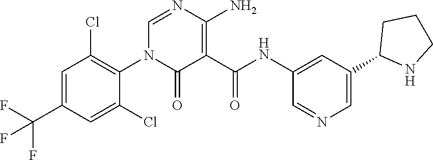 [SMILES: Nc1ncn(-c2c(Cl)cc(C(F)(F)F)cc2Cl)c(=O)c1C(=O)Nc1cncc([C@@H]2CCCN2)c1] |
| 119 | 1 | 527 | 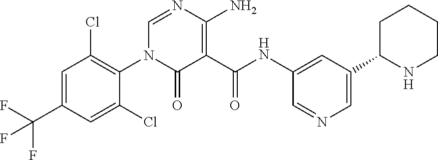 [SMILES: Nc1ncn(-c2c(Cl)cc(C(F)(F)F)cc2Cl)c(=O)c1C(=O)Nc1cncc([C@@H]2CCCCN2)c1] |
| 120 | 1 | 473 | 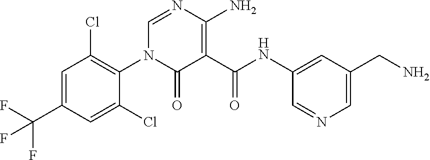 [SMILES: NCc1cncc(NC(=O)c2c(N)ncn(-c3c(Cl)cc(C(F)(F)F)cc3Cl)c2=O)c1] |
| 121 | 1 | 531 | 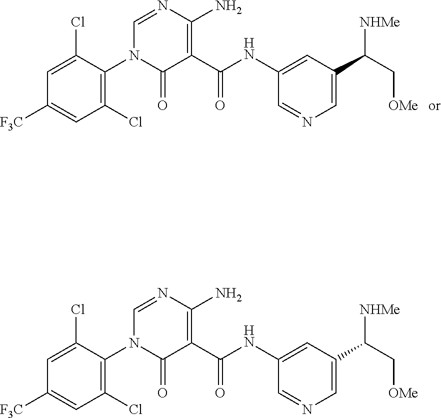 [SMILES: CN[C@@H](COC)c1cncc(NC(=O)c2c(N)ncn(-c3c(Cl)cc(C(F)(F)F)cc3Cl)c2=O)c1 ; CN[C@H](COC)c1cncc(NC(=O)c2c(N)ncn(-c3c(Cl)cc(C(F)(F)F)cc3Cl)c2=O)c1] |
| 122 | 1 | 517 | 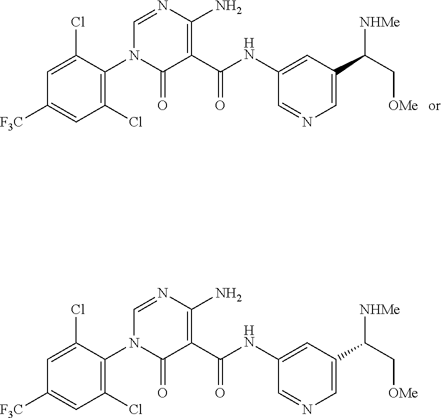 [SMILES: CN[C@@H](COC)c1cncc(NC(=O)c2c(N)ncn(-c3c(Cl)cc(C(F)(F)F)cc3Cl)c2=O)c1 ; CN[C@H](COC)c1cncc(NC(=O)c2c(N)ncn(-c3c(Cl)cc(C(F)(F)F)cc3Cl)c2=O)c1] |
| 123 | 1 | 502 | 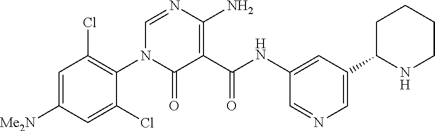 [SMILES: CN(C)c1cc(Cl)c(-n2cnc(N)c(C(=O)Nc3cncc([C@@H]4CCCCN4)c3)c2=O)c(Cl)c1] |
| 124 | 1 | 517 | 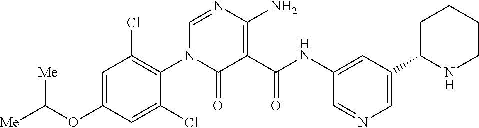 [SMILES: CC(C)Oc1cc(Cl)c(-n2cnc(N)c(C(=O)Nc3cncc([C@@H]4CCCCN4)c3)c2=O)c(Cl)c1] |
| 125 | 1 | 477 | 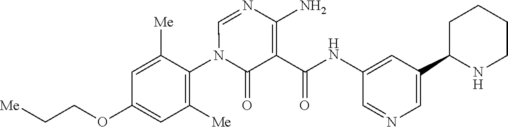 [SMILES: CCCOc1cc(C)c(-n2cnc(N)c(C(=O)Nc3cncc([C@H]4CCCCN4)c3)c2=O)c(C)c1] |
| 126 | 1 | 437 | 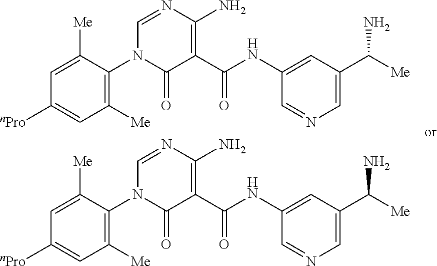 |
| 127 | 1 | 437 | 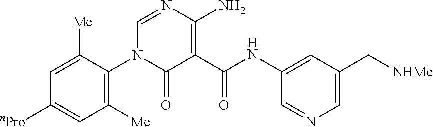 |
| 128 | 1 | 443 | 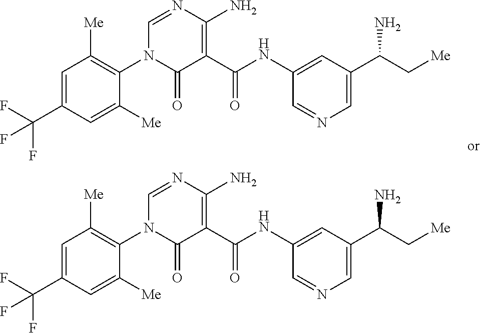 [SMILES: CC[C@@H](N)c1cncc(NC(=O)c2c(N)ncn(-c3c(C)cc(C(F)(F)F)cc3C)c2=O)c1 ; CC[C@H](N)c1cncc(NC(=O)c2c(N)ncn(-c3c(C)cc(C(F)(F)F)cc3C)c2=O)c1] |
| 129 | 1 | 492 | 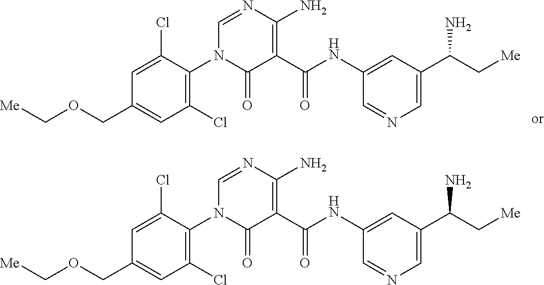 [SMILES: CCOCc1cc(Cl)c(-n2cnc(N)c(C(=O)Nc3cncc([C@H](N)CC)c3)c2=O)c(Cl)c1 ; CCOCc1cc(Cl)c(-n2cnc(N)c(C(=O)Nc3cncc([C@@H](N)CC)c3)c2=O)c(Cl)c1] |
| 130 | 1 | 437 | 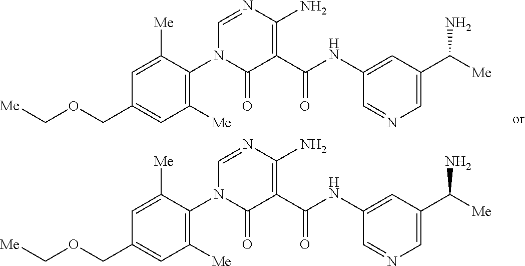 |
| 131 | 1 | 477 | 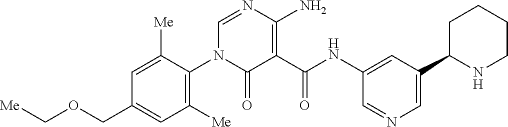 [SMILES: CCOCc1cc(C)c(-n2cnc(N)c(C(=O)Nc3cncc([C@H]4CCCCN4)c3)c2=O)c(C)c1] |
| 132 | 1 | 490 | 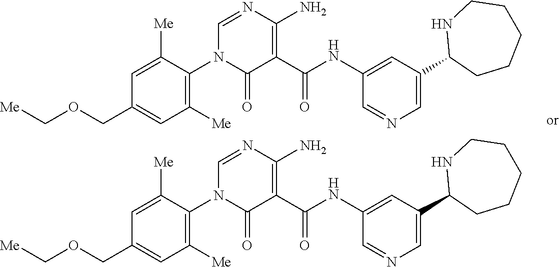 |
| 133 | 1 | 503 | 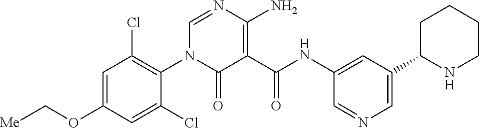 [SMILES: CCOc1cc(Cl)c(-n2cnc(N)c(C(=O)Nc3cncc([C@@H]4CCCCN4)c3)c2=O)c(Cl)c1] |
| 134 | 1 | 490 | 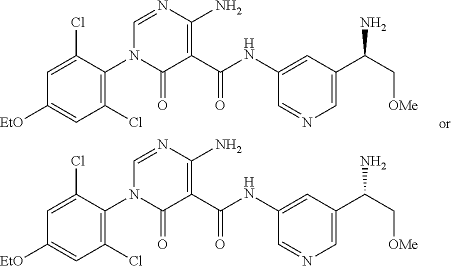 |
| 135 | 1 | 519 | 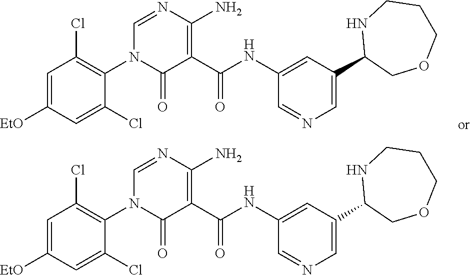 |
| 136 | 1 | 477 | 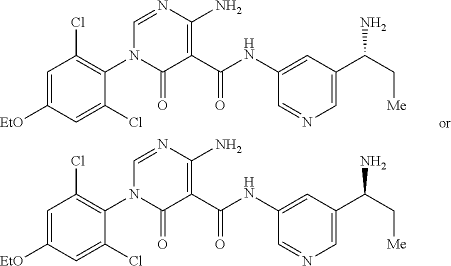 |
| 137 | 1 | 463 | 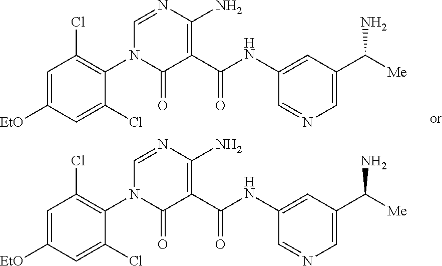 |
| 138 | 1 | 482 | 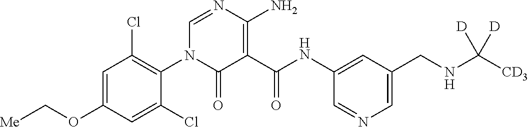 [SMILES: [2H]C([2H])([2H])C([2H])([2H])NCc1cncc(NC(=O)c2c(N)ncn(-c3c(Cl)cc(OCC)cc3Cl)c2=O)c1] |
| 139 | 1 | 489 | 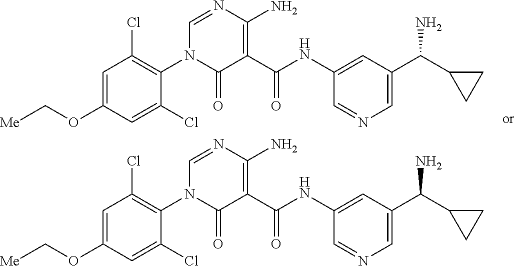 |
| 140 | 1 | 507 | 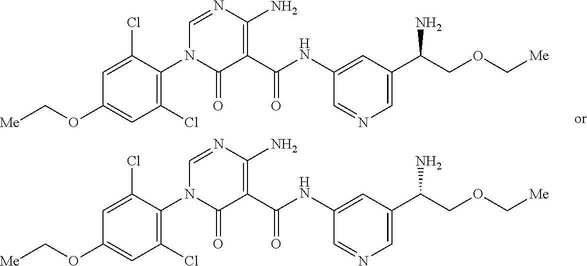 |
| 141 | 1 | 507 | 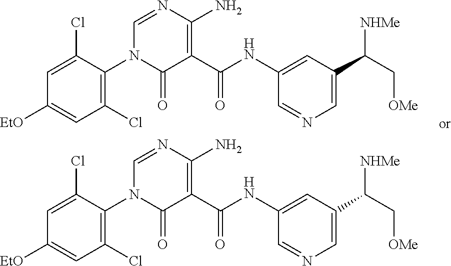 |
| 142 | 1 | 464 | 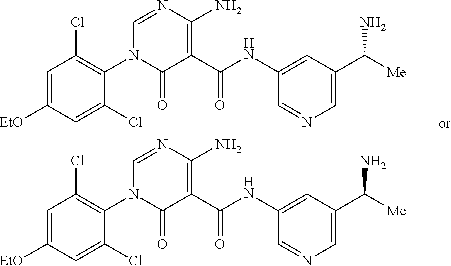 |
| 143 | 1 | 513 | 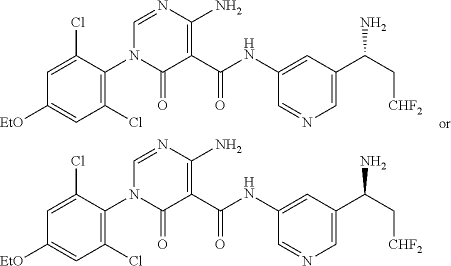 |
| 144 | 1 | 443 | 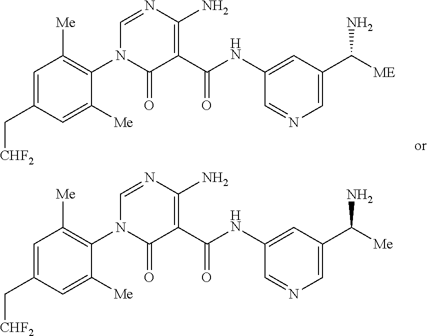 |
| 145 | 1 | 433 | 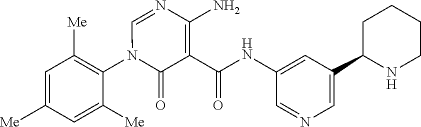 [SMILES: Cc1cc(C)c(-n2cnc(N)c(C(=O)Nc3cncc([C@H]4CCCCN4)c3)c2=O)c(C)c1] |
| 146 | 1 | 447 | 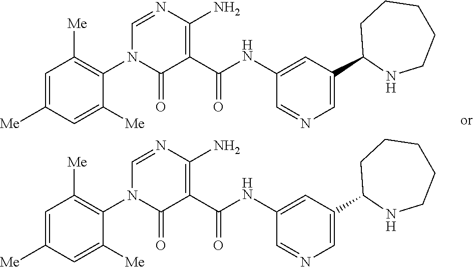 |
| 147 | 1 | 393 | 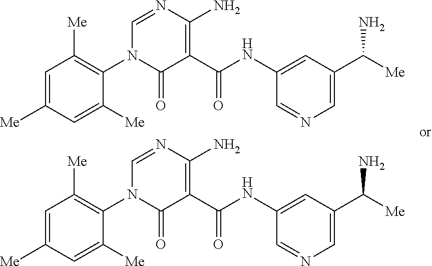 |
| 148 | 1 | 461 | 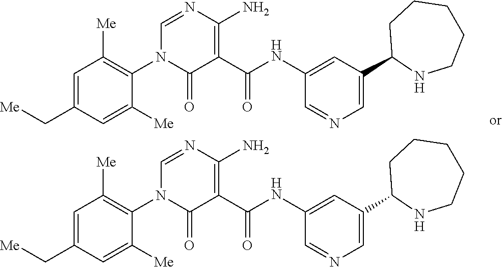 |
| 149 | 1 | 463 | 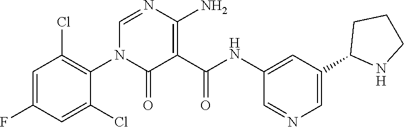 [SMILES: Nc1ncn(-c2c(Cl)cc(F)cc2Cl)c(=O)c1C(=O)Nc1cncc([C@@H]2CCCN2)c1] |
| 150 | 1 | 487 | 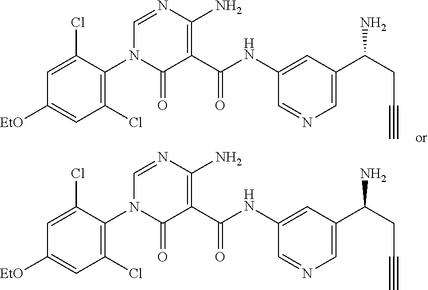 |
| 151 | 1 | 487 | 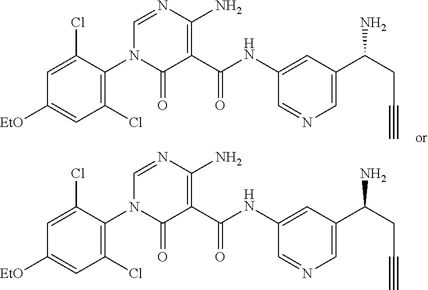 |
| 152 | 1 | 460 | 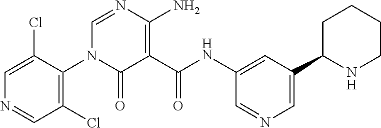 [SMILES: Nc1ncn(-c2c(Cl)cncc2Cl)c(=O)c1C(=O)Nc1cncc([C@H]2CCCCN2)c1] |
| 153 | 1 | 434 | 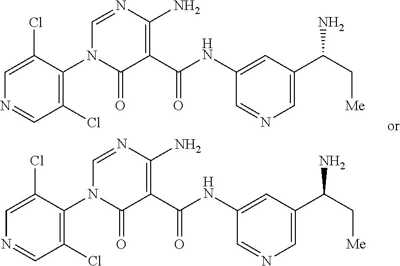 |
| 154 | 1 | 483 | 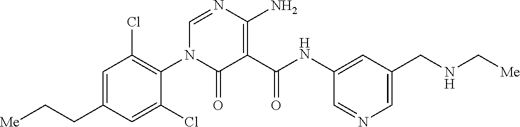 [SMILES: CCCc1cc(Cl)c(-n2cnc(N)c(C(=O)Nc3cncc(CNCC)c3)c2=O)c(Cl)c1] |
| 155 | 1 | 425 | 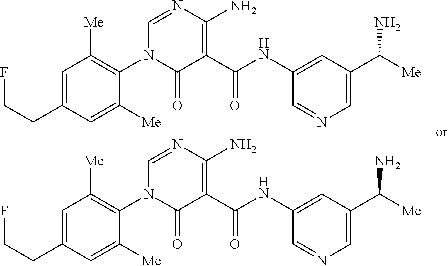 |
| 156 | 1 | 457 | 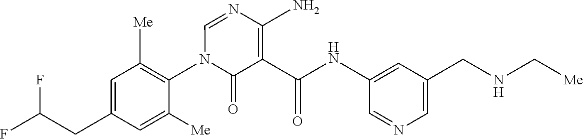 [SMILES: CCNCc1cncc(NC(=O)c2c(N)ncn(-c3c(C)cc(CC(F)F)cc3C)c2=O)c1] |
| 157 | 1 | 449 | 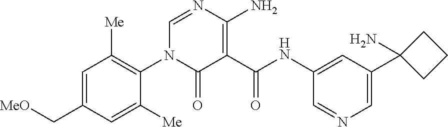 [SMILES: COCc1cc(C)c(-n2cnc(N)c(C(=O)Nc3cncc(C4(N)CCC4)c3)c2=O)c(C)c1] |
| 158 | 1 | 511 | 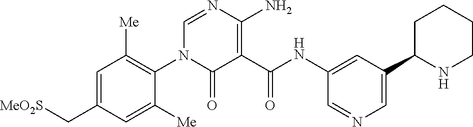 [SMILES: Cc1cc(CS(C)(=O)=O)cc(C)c1-n1cnc(N)c(C(=O)Nc2cncc([C@H]3CCCCN3)c2)c1=O] |
| 159 | 1 | 478 | 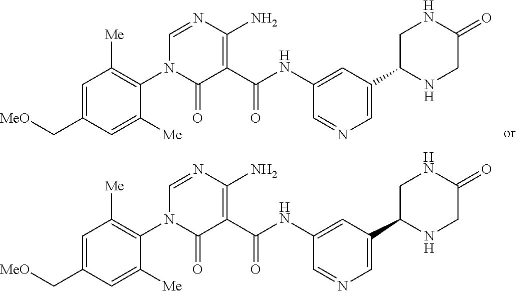 |
| 160 | 1 | 478 | 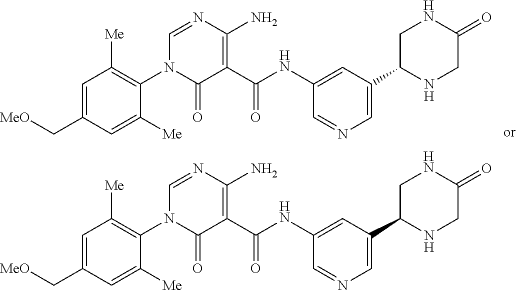 |
| 161 | 1 | 437 | 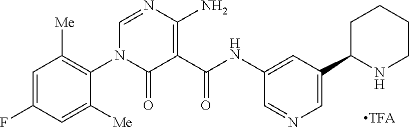 [SMILES: Cc1cc(F)cc(C)c1-n1cnc(N)c(C(=O)Nc2cncc([C@H]3CCCCN3)c2)c1=O] |
| 162 | 1 | 503 | 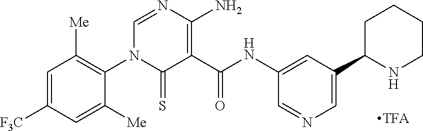 [SMILES: Cc1cc(C(F)(F)F)cc(C)c1-n1cnc(N)c(C(=O)Nc2cncc([C@H]3CCCCN3)c2)c1=S] |
| 163 | 1 | 479 | 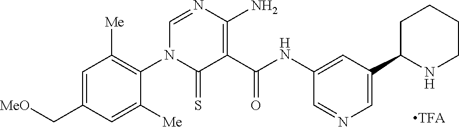 [SMILES: COCc1cc(C)c(-n2cnc(N)c(C(=O)Nc3cncc([C@H]4CCCCN4)c3)c2=S)c(C)c1] |
| 164 | 1 | 442 | 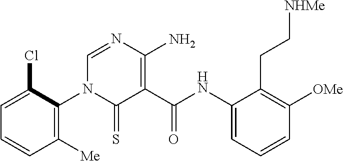 |
| 165 | 1 | 555 | 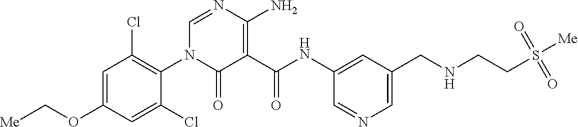 [SMILES: CCOc1cc(Cl)c(-n2cnc(N)c(C(=O)Nc3cncc(CNCCS(C)(=O)=O)c3)c2=O)c(Cl)c1] |
| 166 | 1 | 491 | 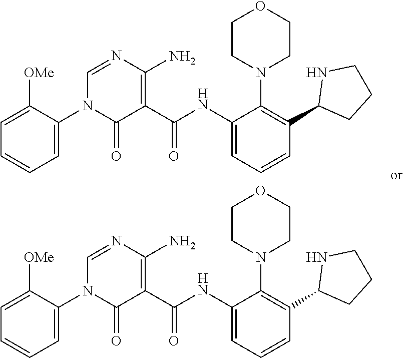 |
| 167 | 1 | | 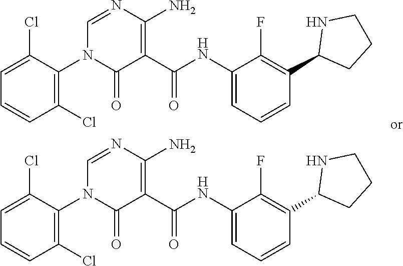 |
| 168 | 1 | 462 | 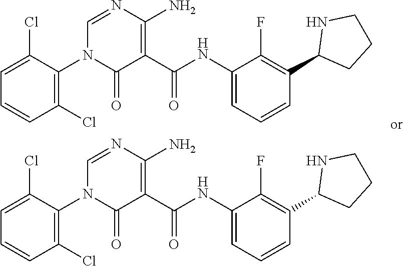 |
| 169 | 1 | | 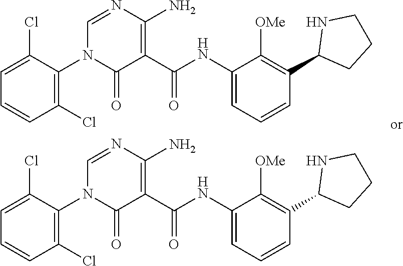 |
| 170 | 1 | 470 | 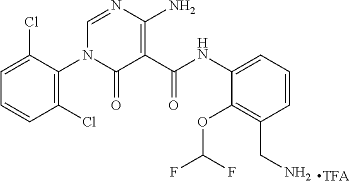 [SMILES: NCc1cccc(NC(=O)c2c(N)ncn(-c3c(Cl)cccc3Cl)c2=O)c1OC(F)F] |
| 171 | 1 | 472 | 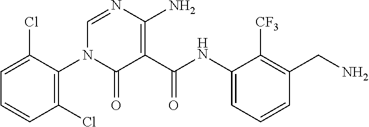 [SMILES: NCc1cccc(NC(=O)c2c(N)ncn(-c3c(Cl)cccc3Cl)c2=O)c1C(F)(F)F] |
| 172 | 1 | 490 | 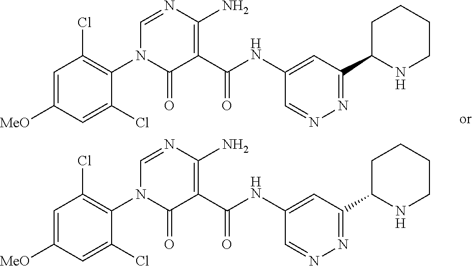 |
| 173 | 1 | 441 | 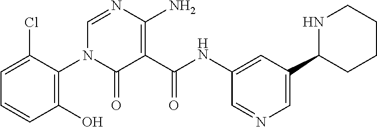 [SMILES: Nc1ncn(-c2c(O)cccc2Cl)c(=O)c1C(=O)Nc1cncc([C@@H]2CCCCN2)c1] |
| 174 | 1 | 437 | 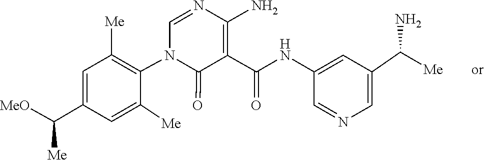 [SMILES: CO[C@H](C)c1cc(C)c(-n2cnc(N)c(C(=O)Nc3cncc([C@@H](C)N)c3)c2=O)c(C)c1] |
| | | | 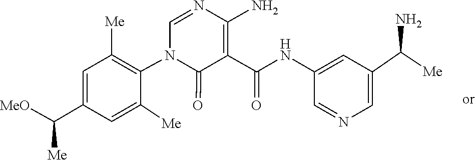 [SMILES: CO[C@H](C)c1cc(C)c(-n2cnc(N)c(C(=O)Nc3cncc([C@H](C)N)c3)c2=O)c(C)c1] |
| | | | 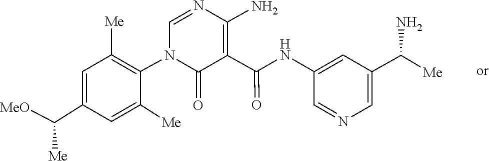 [SMILES: CO[C@@H](C)c1cc(C)c(-n2cnc(N)c(C(=O)Nc3cncc([C@@H](C)N)c3)c2=O)c(C)c1] |
| | | | 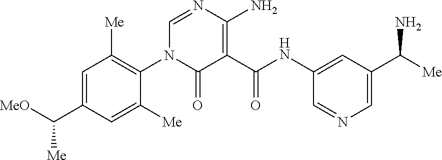 [SMILES: CO[C@@H](C)c1cc(C)c(-n2cnc(N)c(C(=O)Nc3cncc([C@H](C)N)c3)c2=O)c(C)c1] |
| 175 | 1 | 437 | 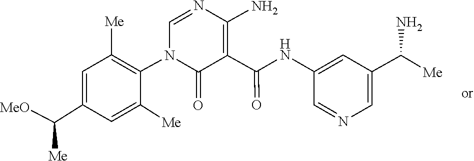 [SMILES: CO[C@H](C)c1cc(C)c(-n2cnc(N)c(C(=O)Nc3cncc([C@@H](C)N)c3)c2=O)c(C)c1] |
| | | | 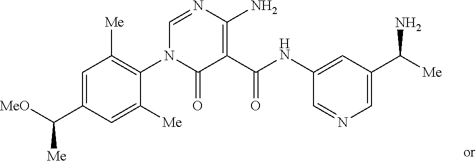 [SMILES: CO[C@H](C)c1cc(C)c(-n2cnc(N)c(C(=O)Nc3cncc([C@H](C)N)c3)c2=O)c(C)c1] |
| | | | 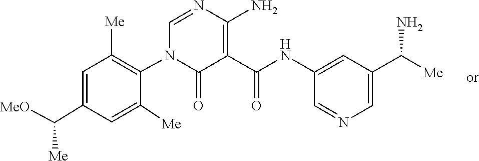 [SMILES: CO[C@@H](C)c1cc(C)c(-n2cnc(N)c(C(=O)Nc3cncc([C@@H](C)N)c3)c2=O)c(C)c1] |
| | | | 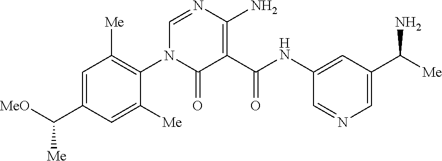 [SMILES: CO[C@@H](C)c1cc(C)c(-n2cnc(N)c(C(=O)Nc3cncc([C@H](C)N)c3)c2=O)c(C)c1] |
| 176 | 1 | 451 | 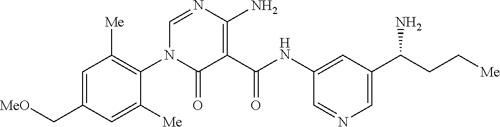 [SMILES: CCC[C@@H](N)c1cncc(NC(=O)c2c(N)ncn(-c3c(C)cc(COC)cc3C)c2=O)c1] |
| 177 | 1 | 467 | 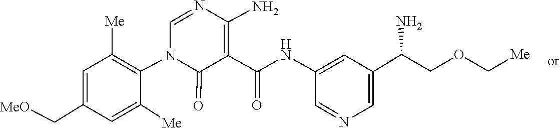 [SMILES: CCOC[C@@H](N)c1cncc(NC(=O)c2c(N)ncn(-c3c(C)cc(COC)cc3C)c2=O)c1] |
| | | | 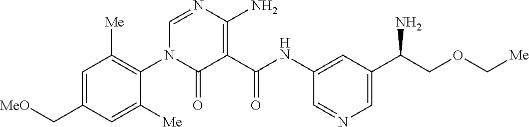 [SMILES: CCOC[C@H](N)c1cncc(NC(=O)c2c(N)ncn(-c3c(C)cc(COC)cc3C)c2=O)c1] |
| 178 | 1 | 477 | 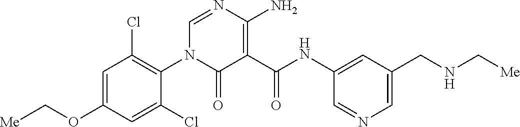 [SMILES: CCNCc1cncc(NC(=O)c2c(N)ncn(-c3c(Cl)cc(OCC)cc3Cl)c2=O)c1] |
| 179 | 1 | 475 | 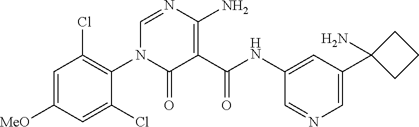 [SMILES: COc1cc(Cl)c(-n2cnc(N)c(C(=O)Nc3cncc(C4(N)CCC4)c3)c2=O)c(Cl)c1] |
| 180 | 1 | 458 | 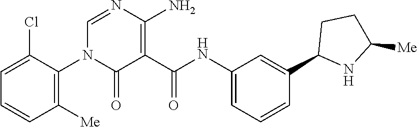 [SMILES: Cc1cccc(Cl)c1-n1cnc(N)c(C(=O)Nc2cccc([C@H]3CC[C@@H](C)N3)c2)c1=O] |
| 181 | 1 | 461 | 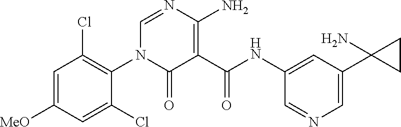 [SMILES: COc1cc(Cl)c(-n2cnc(N)c(C(=O)Nc3cncc(C4(N)CC4)c3)c2=O)c(Cl)c1] |
| 182 | 1 | 505 | 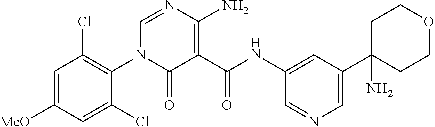 [SMILES: COc1cc(Cl)c(-n2cnc(N)c(C(=O)Nc3cncc(C4(N)CCOCC4)c3)c2=O)c(Cl)c1] |
| 183 | 1 | 489 | 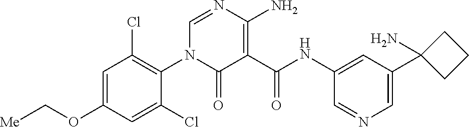 [SMILES: CCOc1cc(Cl)c(-n2cnc(N)c(C(=O)Nc3cncc(C4(N)CCC4)c3)c2=O)c(Cl)c1] |
| 184 | 1 | 465 | 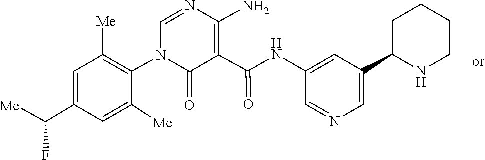 [SMILES: Cc1cc([C@@H](C)F)cc(C)c1-n1cnc(N)c(C(=O)Nc2cncc([C@H]3CCCCN3)c2)c1=O] |
| | | | 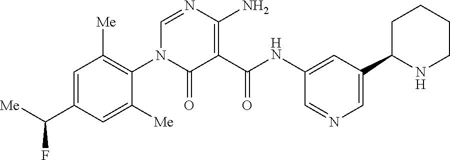 [SMILES: Cc1cc([C@H](C)F)cc(C)c1-n1cnc(N)c(C(=O)Nc2cncc([C@H]3CCCCN3)c2)c1=O] |
| 185 | 1 | 465 | 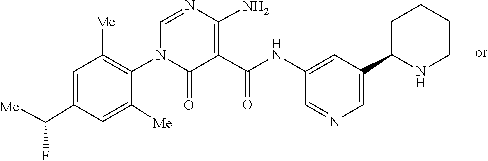 [SMILES: Cc1cc([C@@H](C)F)cc(C)c1-n1cnc(N)c(C(=O)Nc2cncc([C@H]3CCCCN3)c2)c1=O] |
| | | | 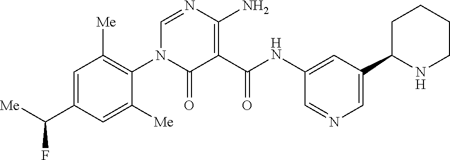 [SMILES: Cc1cc([C@H](C)F)cc(C)c1-n1cnc(N)c(C(=O)Nc2cncc([C@H]3CCCCN3)c2)c1=O] |
| 186 | 1 | 483 | 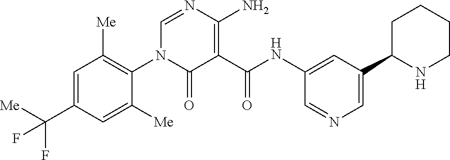 [SMILES: Cc1cc(C(C)(F)F)cc(C)c1-n1cnc(N)c(C(=O)Nc2cncc([C@H]3CCCCN3)c2)c1=O] |
| 187 | 1 | 505 | 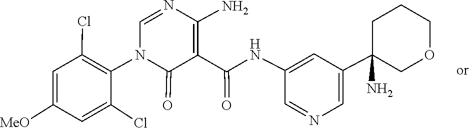 [SMILES: COc1cc(Cl)c(-n2cnc(N)c(C(=O)Nc3cncc([C@]4(N)CCCOC4)c3)c2=O)c(Cl)c1] |
| | | | 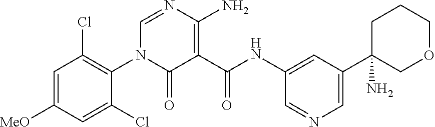 [SMILES: COc1cc(Cl)c(-n2cnc(N)c(C(=O)Nc3cncc([C@@]4(N)CCCOC4)c3)c2=O)c(Cl)c1] |
| 188 | 1 | 505 | 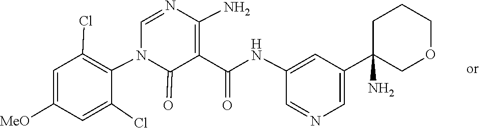 [SMILES: COc1cc(Cl)c(-n2cnc(N)c(C(=O)Nc3cncc([C@]4(N)CCCOC4)c3)c2=O)c(Cl)c1] |
| | | | 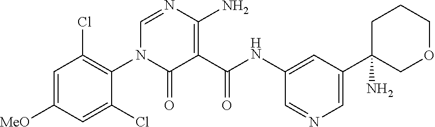 [SMILES: COc1cc(Cl)c(-n2cnc(N)c(C(=O)Nc3cncc([C@@]4(N)CCCOC4)c3)c2=O)c(Cl)c1] |
| 189 | 1 | 473 | 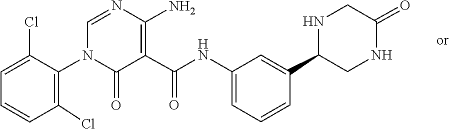 [SMILES: Nc1ncn(-c2c(Cl)cccc2Cl)c(=O)c1C(=O)Nc1cccc([C@@H]2CNC(=O)CN2)c1] |
| | | | 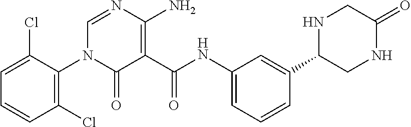 [SMILES: Nc1ncn(-c2c(Cl)cccc2Cl)c(=O)c1C(=O)Nc1cccc([C@H]2CNC(=O)CN2)c1] |
| 190 | 1 | 473 | 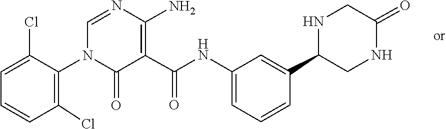 [SMILES: Nc1ncn(-c2c(Cl)cccc2Cl)c(=O)c1C(=O)Nc1cccc([C@@H]2CNC(=O)CN2)c1] |
| | | | 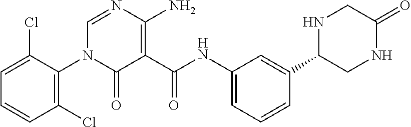 [SMILES: Nc1ncn(-c2c(Cl)cccc2Cl)c(=O)c1C(=O)Nc1cccc([C@H]2CNC(=O)CN2)c1] |
| 191 | 1 | 474 | 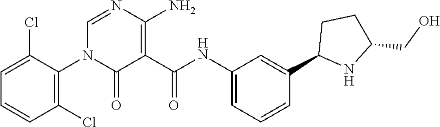 [SMILES: Nc1ncn(-c2c(Cl)cccc2Cl)c(=O)c1C(=O)Nc1cccc([C@H]2CC[C@H](CO)N2)c1] |
| | | | 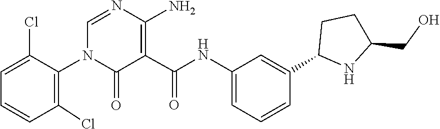 [SMILES: Nc1ncn(-c2c(Cl)cccc2Cl)c(=O)c1C(=O)Nc1cccc([C@@H]2CC[C@@H](CO)N2)c1] |
| 192 | 1 | 501 | 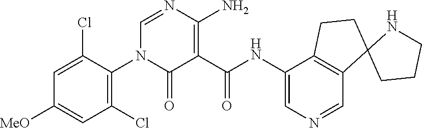 [SMILES: COc1cc(Cl)c(-n2cnc(N)c(C(=O)Nc3cncc4c3CCC43CCCN3)c2=O)c(Cl)c1] |
| 193 | 1 & FGI | 510 | 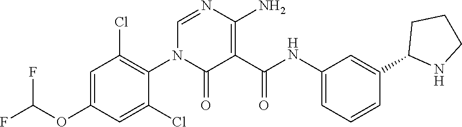 [SMILES: Nc1ncn(-c2c(Cl)cc(OC(F)F)cc2Cl)c(=O)c1C(=O)Nc1cccc([C@@H]2CCCN2)c1] |
| 194 | 1 & FGI | 501 | 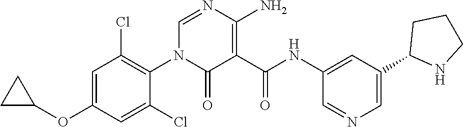 [SMILES: Nc1ncn(-c2c(Cl)cc(OC3CC3)cc2Cl)c(=O)c1C(=O)Nc1cncc([C@@H]2CCCN2)c1] |
| 195 | 1 & FGI | 489 | 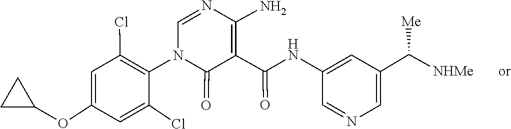 [SMILES: CN[C@@H](C)c1cncc(NC(=O)c2c(N)ncn(-c3c(Cl)cc(OC4CC4)cc3Cl)c2=O)c1] |
| | | | 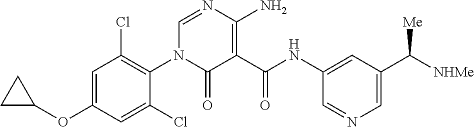 [SMILES: CN[C@H](C)c1cncc(NC(=O)c2c(N)ncn(-c3c(Cl)cc(OC4CC4)cc3Cl)c2=O)c1] |
| 196 | 1 & FGI | 517 | 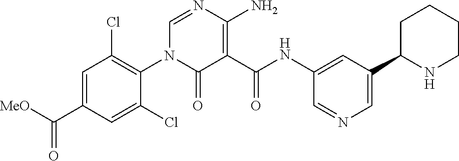 [SMILES: COC(=O)c1cc(Cl)c(-n2cnc(N)c(C(=O)Nc3cncc([C@H]4CCCCN4)c3)c2=O)c(Cl)c1] |
| 197 | 1 | 463 | 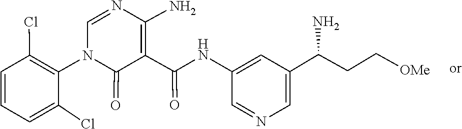 [SMILES: COCC[C@@H](N)c1cncc(NC(=O)c2c(N)ncn(-c3c(Cl)cccc3Cl)c2=O)c1] |
| | | | 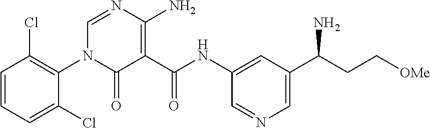 [SMILES: COCC[C@H](N)c1cncc(NC(=O)c2c(N)ncn(-c3c(Cl)cccc3Cl)c2=O)c1] |
| 198 | 1 & FGI | 491 | 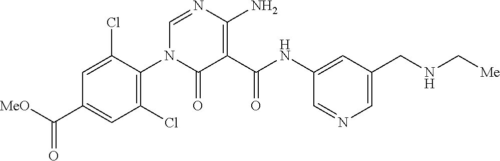 [SMILES: CCNCc1cncc(NC(=O)c2c(N)ncn(-c3c(Cl)cc(C(=O)OC)cc3Cl)c2=O)c1] |
| 199 | 1 | 505 | 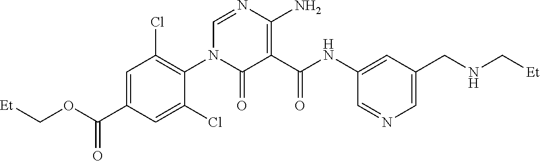 [SMILES: CCCNCc1cncc(NC(=O)c2c(N)ncn(-c3c(Cl)cc(C(=O)OCCC)cc3Cl)c2=O)c1] |
| 200 | 1 & FGI | 500 | 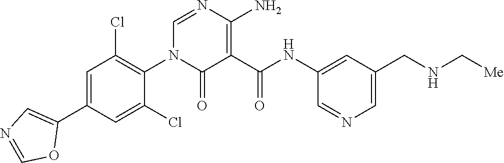 [SMILES: CCNCc1cncc(NC(=O)c2c(N)ncn(-c3c(Cl)cc(-c4cnco4)cc3Cl)c2=O)c1] |
| 201 | 1 & FGI | 500 | 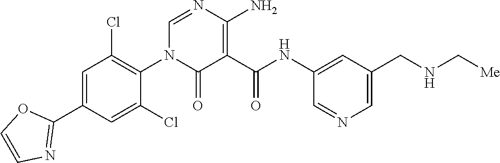 [SMILES: CCNCc1cncc(NC(=O)c2c(N)ncn(-c3c(Cl)cc(-c4ncco4)cc3Cl)c2=O)c1] |
| 202 | 1 & FGI | 515 | 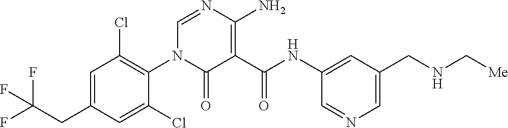 [SMILES: CCNCc1cncc(NC(=O)c2c(N)ncn(-c3c(Cl)cc(CC(F)(F)F)cc3Cl)c2=O)c1] |
| 203 | 1 & FGI | 497 | 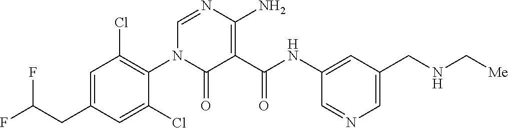 [SMILES: CCNCc1cncc(NC(=O)c2c(N)ncn(-c3c(Cl)cc(CC(F)F)cc3Cl)c2=O)c1] |
| 204 | 1 & Deprotection | 485 | 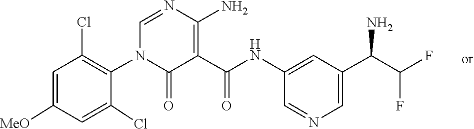 [SMILES: COc1cc(Cl)c(-n2cnc(N)c(C(=O)Nc3cncc([C@@H](N)C(F)F)c3)c2=O)c(Cl)c1] |
| | | | 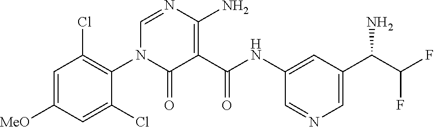 [SMILES: COc1cc(Cl)c(-n2cnc(N)c(C(=O)Nc3cncc([C@H](N)C(F)F)c3)c2=O)c(Cl)c1] |
| 205 | 1 & FGI | 449 | 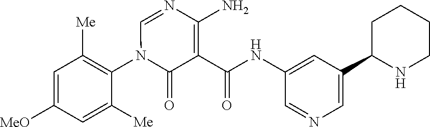 [SMILES: COc1cc(C)c(-n2cnc(N)c(C(=O)Nc3cncc([C@H]4CCCCN4)c3)c2=O)c(C)c1] |
| 206 | 1 & Double deprotection | 420 | 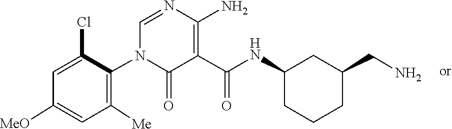 |
| | | | 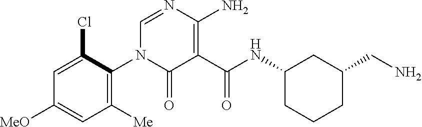 |
| 207 | 1 & FGI | 477 | 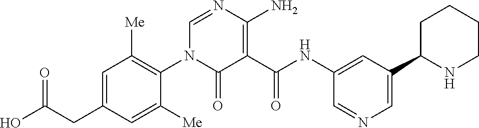 [SMILES: Cc1cc(CC(=O)O)cc(C)c1-n1cnc(N)c(C(=O)Nc2cncc([C@H]3CCCCN3)c2)c1=O] |
| 208 | 1 | 505 | 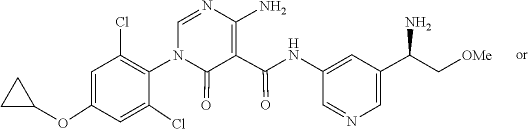 [SMILES: COC[C@H](N)c1cncc(NC(=O)c2c(N)ncn(-c3c(Cl)cc(OC4CC4)cc3Cl)c2=O)c1] |
| | | | 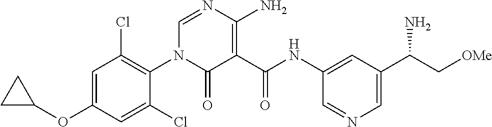 [SMILES: COC[C@@H](N)c1cncc(NC(=O)c2c(N)ncn(-c3c(Cl)cc(OC4CC4)cc3Cl)c2=O)c1] |
| 209 | 1 & FGI | 514 | 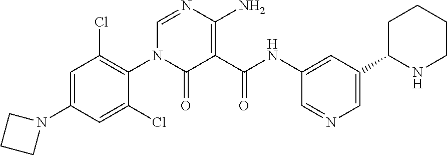 [SMILES: Nc1ncn(-c2c(Cl)cc(N3CCC3)cc2Cl)c(=O)c1C(=O)Nc1cncc([C@@H]2CCCCN2)c1] |
| 210 | 1 | 476 | 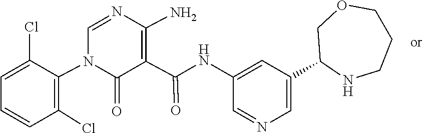 [SMILES: Nc1ncn(-c2c(Cl)cccc2Cl)c(=O)c1C(=O)Nc1cncc([C@@H]2COCCCN2)c1] |
| | | | 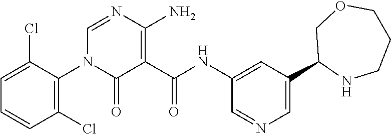 [SMILES: Nc1ncn(-c2c(Cl)cccc2Cl)c(=O)c1C(=O)Nc1cncc([C@H]2COCCCN2)c1] |
| 211 | 1 | 474 | 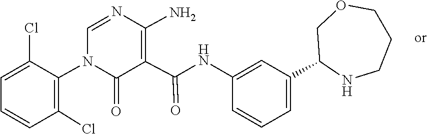 [SMILES: Nc1ncn(-c2c(Cl)cccc2Cl)c(=O)c1C(=O)Nc1cccc([C@@H]2COCCCN2)c1] |
| | | | 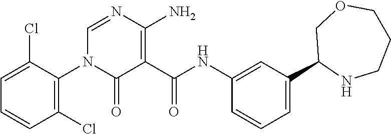 [SMILES: Nc1ncn(-c2c(Cl)cccc2Cl)c(=O)c1C(=O)Nc1cccc([C@H]2COCCCN2)c1] |
| 212 | 1 | 492 | 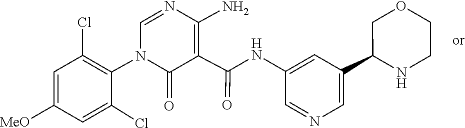 [SMILES: COc1cc(Cl)c(-n2cnc(N)c(C(=O)Nc3cncc([C@H]4COCCN4)c3)c2=O)c(Cl)c1] |
| | | | 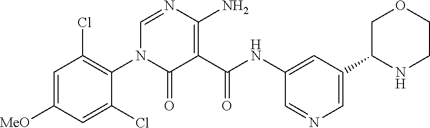 [SMILES: COc1cc(Cl)c(-n2cnc(N)c(C(=O)Nc3cncc([C@@H]4COCCN4)c3)c2=O)c(Cl)c1] |
| 213 | 1 | 461 | 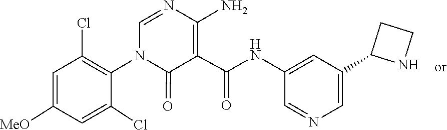 [SMILES: COc1cc(Cl)c(-n2cnc(N)c(C(=O)Nc3cncc([C@@H]4CCN4)c3)c2=O)c(Cl)c1] |
| | | | 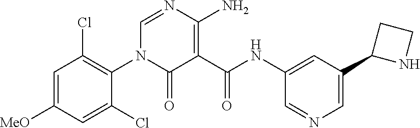 [SMILES: COc1cc(Cl)c(-n2cnc(N)c(C(=O)Nc3cncc([C@H]4CCN4)c3)c2=O)c(Cl)c1] |
| 214 | 1 | 475 | 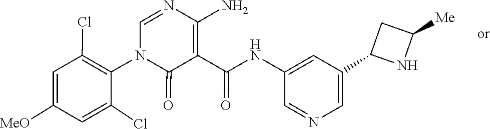 [SMILES: COc1cc(Cl)c(-n2cnc(N)c(C(=O)Nc3cncc([C@@H]4C[C@@H](C)N4)c3)c2=O)c(Cl)c1] |
| | | | 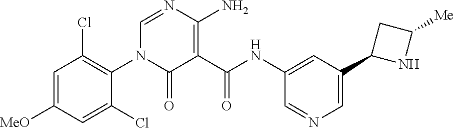 [SMILES: COc1cc(Cl)c(-n2cnc(N)c(C(=O)Nc3cncc([C@H]4C[C@H](C)N4)c3)c2=O)c(Cl)c1] |
| 215 | 1 | 475 | 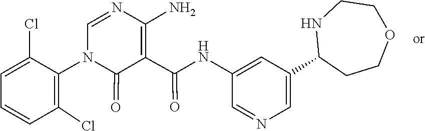 [SMILES: Nc1ncn(-c2c(Cl)cccc2Cl)c(=O)c1C(=O)Nc1cncc([C@H]2CCOCCN2)c1] |
| | | | 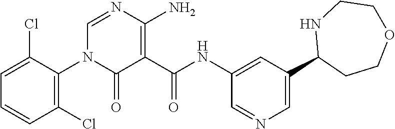 [SMILES: Nc1ncn(-c2c(Cl)cccc2Cl)c(=O)c1C(=O)Nc1cncc([C@@H]2CCOCCN2)c1] |
| 216 | 1 | 487 | 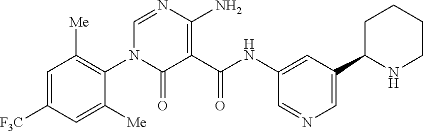 [SMILES: Cc1cc(C(F)(F)F)cc(C)c1-n1cnc(N)c(C(=O)Nc2cncc([C@H]3CCCCN3)c2)c1=O] |
| 217 | 1 | 442 | 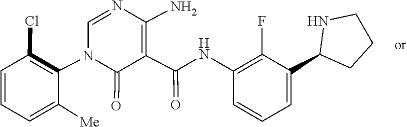 |
| | | | 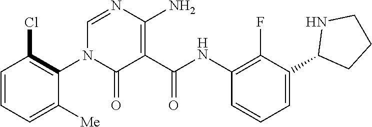 |
| 218 | 1 | 407 | 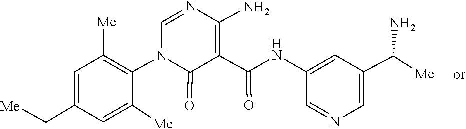 [SMILES: CCc1cc(C)c(-n2cnc(N)c(C(=O)Nc3cncc([C@@H](C)N)c3)c2=O)c(C)c1] |
| | | | 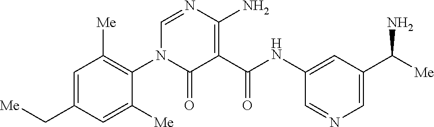 [SMILES: CCc1cc(C)c(-n2cnc(N)c(C(=O)Nc3cncc([C@H](C)N)c3)c2=O)c(C)c1] |
| 219 | 1 | 464 | 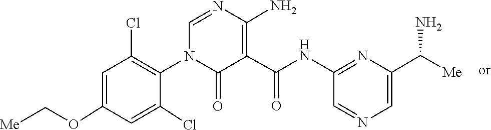 [SMILES: CCOc1cc(Cl)c(-n2cnc(N)c(C(=O)Nc3cncc([C@@H](C)N)n3)c2=O)c(Cl)c1] |
| | | | 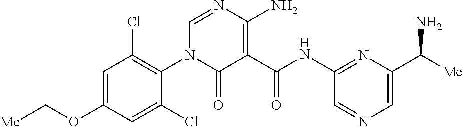 [SMILES: CCOc1cc(Cl)c(-n2cnc(N)c(C(=O)Nc3cncc([C@H](C)N)n3)c2=O)c(Cl)c1] |
| 220 | 1 | 448 | 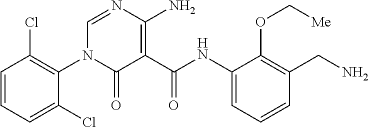 [SMILES: CCOc1c(CN)cccc1NC(=O)c1c(N)ncn(-c2c(Cl)cccc2Cl)c1=O] |
| 221 | 1 & FGI | 434 | 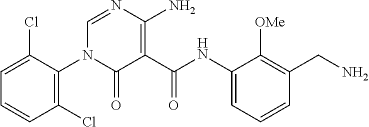 [SMILES: COc1c(CN)cccc1NC(=O)c1c(N)ncn(-c2c(Cl)cccc2Cl)c1=O] |
| 222 | 1 | 506 | 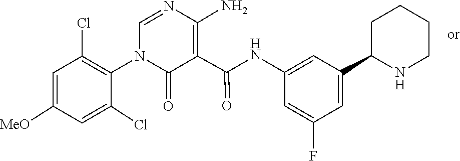 [SMILES: COc1cc(Cl)c(-n2cnc(N)c(C(=O)Nc3cc(F)cc([C@H]4CCCCN4)c3)c2=O)c(Cl)c1] |
| | | | 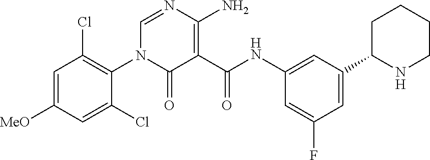 [SMILES: COc1cc(Cl)c(-n2cnc(N)c(C(=O)Nc3cc(F)cc([C@@H]4CCCCN4)c3)c2=O)c(Cl)c1] |

### Definitions

As used herein, the term “pharmaceutically acceptable salt” refers to pharmaceutical salts that are, within the scope of sound medical judgment, suitable for use in contact with the tissues of humans and lower animals without undue toxicity, irritation, and allergic response, and are commensurate with a reasonable benefit/risk ratio. Pharmaceutically acceptable salts are well known in the art. For example, S. M. Berge et al. describes pharmacologically acceptable salts in J. Pharm. Sci. (1977) 66:1-19. Compounds of the present teachings with basic groups can form pharmaceutically acceptable salts with pharmaceutically acceptable acid(s). Suitable pharmaceutically acceptable acid addition salts of the compounds described herein include salts of inorganic acids (such as hydrochloric acid, hydrobromic, phosphoric, nitric, and sulfuric acids) and of organic acids (such as acetic acid, benzenesulfonic, benzoic, methanesulfonic, and p-toluenesulfonic acids). Compounds of the present teachings with acidic groups can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

The term “alkyl” used alone or as part of a larger moiety, such as “alkoxy”, “hydroxyalkyl” and the like, means a saturated aliphatic straight-chain or branched monovalent hydrocarbon radical. Unless otherwise specified, an alkyl group typically has 1 to 6 carbon atoms (C₁-C₆alkyl), alternatively, 1 to 3 carbon atoms (C₁-C₃alkyl). “C₁-C₆alkyl” means a radical having 1 to 6 carbon atoms in a linear or branched arrangement, such as methyl, ethyl, propyl, isopropyl, butyl, isobutyl, tert-butyl, and the like.

“Alkoxy” means an alkyl radical attached through an oxygen linking atom, represented by —O-alkyl, wherein alkyl is as defined above. For example, “C₁-C₆alkoxy” includes methoxy, ethoxy, propoxy, butoxy, pentoxy, isopentoxy, isopropoxy, and hexoxy.

“Alkynyl” refers to a branched or unbranched hydrocarbon moiety containing at least one triple bond. Unless otherwise specified, an alkynyl group comprises 2 to 6 carbon atoms, or 2 to 4 carbon atoms. Representative examples of alkynyl include, but are not limited to, ethynyl, propynyl, 1-butynyl, 2-butynyl, 1-methypropynyl, 2-methypropynyl, 3-methypropynyl and the like.

“Alkenyl” refers to a branched or unbranched hydrocarbon moiety containing at least one double bond. Unless otherwise specified, an alkenyl group comprises 2 to 6 carbon atoms, or 2 to 4 carbon atoms. Representative examples of alkenyl include, but are not limited to, ethenyl, propenyl, 1-butenyl, 2-butenyl, 1-methypropenyl, and the like.

“Aryl”, when used alone or as part of another moiety such as aralkyl, refers to an aromatic hydrocarbon of six to 10 ring atoms, such as phenyl or naphthyl.

The term “halogen”, “halo”, or “hal” means fluorine or fluoro (F), chlorine or chloro (C₁), bromine or bromo (Br), or iodine or iodo (I).

“Cycloalkyl” means a saturated aliphatic cyclic hydrocarbon ring radical. Unless otherwise specified, a cycloalkyl has 3 to 8 ring carbon atoms (C₃-C₈cycloalkyl) (i.e., 3, 4, 5, 6, 7, 8, 9, or 10), alternatively, 3 to 6 ring carbon atoms (C₃-C₉cycloalkyl) (i.e., 3, 4, 5, or 6), alternatively, 3 to 5 carbon atoms (C₃-C₈cycloalkyl) (i.e., 3, 4, or 5). “C₃-C₆Cycloalkyl” means a radical having from 3 to 6 carbon atoms arranged in a monocyclic ring. A C₃₋₆ cycloalkyl includes cyclopropyl, cyclobutyl, cyclopentyl, and cyclohexyl. A C₃-C₅ cycloalkyl includes cyclopropyl, cyclobutyl, and cyclopentyl.

The term “heterocyclyl” refers to a monocyclic or bicyclic non-aromatic ring radical containing unless otherwise specified, 3 to 12 or 3 to 8 ring atoms (i.e., “3, 4, 5, 6, 7, or 8 membered”) selected from carbon atom and 1 to 4 heteroatoms. Each heteroatom is independently selected from nitrogen, quaternary nitrogen, oxidized nitrogen (e.g., NO); oxygen; and sulfur, including sulfoxide and sulfone. Representative heterocycles include piperidinyl, 1,4-oxazepanyl, azetidinyl, morpholinyl, pyrrolidinyl, piperazinyl, piperazine-2-onyl, octahydrocyclopenta[c]pyrrolyl, tetrahydrofuranyl, tetrahydropyranyl, azepanyl, and the like. In some embodiments, the heterocyclyl is a saturated monocyclic ring. In some embodiments, the heterocyclyl is a saturated bicyclic ring.

“Heteroaryl” refers to an aromatic 4- to 12-membered monocyclic or bicyclic ring system, having 1 to 4 heteroatoms independently selected from O, N, S, and NR^{d}, and wherein N can be oxidized (e.g., N(O)) or quaternized, and S can be optionally oxidized to sulfoxide and sulfone. A monocyclic heteroaryl has 5 or 6 ring atoms, i.e., is 5 to 6 membered. Examples of 5- to 6-membered monocyclic heteroaryls include, but are not limited to, pyrrolyl, furanyl, thiophenyl (or thienyl), imidazolyl, pyrazolyl, oxazolyl, isoxazolyl, thiazolyl, pyridinyl, pyranyl, pyrazinyl, pyrimidinyl, triazinyl, tetrazinyl, and the like. A bicyclic heteroaryl has 8 to 10 ring atoms, i.e., is 8 to 10 membered. Examples of 8- to 10-membered bicyclic heteroaryls include, but are not limited to indolyl, benzimidazolyl, benzoxazolyl, benzothiazolyl, benzofuranyl, benzothiofuranyl, quinolinyl, isoquinolinyl and the like.

The term “substituted”, whether preceded by the term “optionally” or not, refers to the replacement of a hydrogen substituent in a given structure with a non-hydrogen substituent. Thus, for example, a substituted alkyl is an alkyl wherein at least one non-hydrogen substituent is in the place of a hydrogen substituent on the alkyl group. To illustrate, monofluoroalkyl is an alkyl substituted with a fluoro substituent, and difluoroalkyl is an alkyl substituted with two fluoro substituents. It should be recognized that if there is more than one substitution on a substituent, each non-hydrogen substituent can be identical or different (unless otherwise stated).

If a group is described as being “optionally substituted”, the group can be either (1) not substituted or (2) substituted.

If a group is described as being optionally substituted with up to a particular number of non-hydrogen substituents, that group can be either (1) not substituted; or (2) substituted by up to that particular number of non-hydrogen substituents or by up to the maximum number of substitutable positions on the substituent, whichever is less. Thus, for example, if a group is described as a cycloalkyl optionally substituted with up to 3 non-hydrogen substituents, then any cycloalkyl with less than 3 substitutable positions would be optionally substituted by up to only as many non-hydrogen substituents as the cycloalkyl has substitutable positions.

Compounds having one or more chiral centers can exist in various stereoisomeric forms, i.e., each chiral center can have an R or S configuration or can be a mixture of both. Stereoisomers are compounds that differ only in their spatial arrangement. Stereoisomers include all diastereomeric and enantiomeric forms of a compound. Enantiomers are stereoisomers that are mirror images of each other. Diastereomers are stereoisomers having two or more chiral centers that are not identical and are not mirror images of each other.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9% (except when the designation “rac” or “racemate accompanies the structure or name, as explained in the following two paragraphs). “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When the stereochemical configuration at a chiral center in a compound is depicted by chemical name (e.g., where the configuration is indicated in the name by “R” or “S”) or structure (e.g., the configuration is indicated by “wedge” bonds) and the designation “rac” or “racemate” accompanies the structure or is designated in the chemical name, a racemic mixture is intended.

When two or more stereoisomers are depicted by their chemical names or structures, and the names or structures are connected by an “or”, one or the other of the two or more stereoisomers is intended, but not both.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

A racemic mixture means a mixture of 50% of one enantiomer and 50% of its corresponding enantiomer. The present teachings encompass all enantiomerically-pure, enantiomerically-enriched, diastereomerically pure, diastereomerically enriched, and racemic mixtures, and diastereomeric mixtures of the compounds described herein.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

“Peak 1” or “first eluting isomer” in the Experimental section refers to an intended reaction product compound obtained from a chromatography separation/purification that elutes earlier than a second intended reaction product compound from the same preceding reaction. The second intended product compound is referred to as “peak 2” or “second eluting isomer”.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that, unless otherwise indicated, one of the encompassed stereoisomers or any mixture of the encompassed stereoisomers are included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

The number of carbon atoms in a group is specified herein by the prefix “Cₓ-Cₓₓ”, wherein x and xx are integers. For example, “C₁-C₃alkyl” is an alkyl group which has from 1 to 3 carbon atoms.

The suffix “yl” added to the end of a chemical name indicates that the named moiety is bonded to the molecule at one point, i.e., monovalent. The suffix “ene” added to the end of a chemical name indicates that the named moiety is bonded to the molecule at two points, i.e., bivalent.

### Methods of Use

Compounds of the disclosure are GSK3 inhibitors, more specifically, GSK3α inhibitors. The use of the word “inhibitor” means that a compound or a pharmaceutically acceptable salt thereof inhibits activity of GSK3 (e.g., GSK3α). By “inhibit” herein is meant to decrease the activity of the target enzyme as compared to the activity of that enzyme in the absence of the inhibitor. In some alternatives, the term “inhibit” means a decrease in GSK3 (e.g., GSK3α) activity of at least 5%, at least 10%, at least 20%, at least 50%, at least 60%, at least 79%, at least 80%, at least 90% or at least 95%. In other alternatives, inhibit means a decrease in GSK3α activity of 5% to 25%, 25% to 50%, 50 to 70%, 75 to 100%. In some embodiments, inhibit means a decrease in GSK3 (e.g., GSK3α) activity about 95% to 100%, e.g., a decrease in activity of 95%, 96%, 97%, 98%, 99%, or 100%. Such decreases can be measured using a variety of techniques that would be recognizable by one of skill in the art, including in vitro kinase assays.

Compounds of the disclosure are selective GSK3α inhibitors. As used herein, a “selective GSK3α inhibitor” refers to a compound or a pharmaceutically acceptable salt thereof that has the ability to selectively inhibit GSK3α kinase over other targets. More specifically, a selective GSK3α inhibitor has the ability to selectively inhibit GSK3α over another kinase. A selective GSK3α inhibitor has the ability to selectively reduce target signaling activity relative to off-target signaling activity, via direct or indirect interaction with the target. The ability to selectively target GSK3× with a compound or pharmaceutically acceptable salt thereof provides advantages in terms of improved potency, less off-target activity and an increased probability of clinical success in comparison with a non-selective compound or salt. A GSK3α inhibitor that selectively inhibits GSK3α may have an activity that is at least 2-fold relative to another kinase (e.g., at least 10-fold; at least 15-fold; at least 20-fold; at least 30-fold; at least 40-fold selectivity; at least 50-fold; at least 60-fold; at least 70-fold; at least 80-fold; at least 90-fold; at least 100-fold; at least 125-fold; at least 150-fold; at least 175-fold; or at least 200-fold. In some alternatives, a selective GSK3α inhibitor exhibits at least 15-fold selectivity over another kinase, e.g., GSK3B. In some alternatives, the selective GSK3α inhibitors are selective over GSK3B.

The disclosure provides methods of modulating (e.g., inhibiting) GSK3 (more specifically GSK3) activity in a patient in need thereof, said method comprising administering to the patient a compound provided herein, or a pharmaceutically acceptable salt thereof. In certain embodiments, the compounds of the disclosure, or pharmaceutically acceptable salts thereof, are useful for therapeutic administration to enhance, stimulate and/or increase immunity in patients in need thereof, e.g., in cancer patients or patients with viral infection, diabetes, Alzheimer's disease and other CNS disorders.

In some instances, the compounds of the disclosure, or pharmaceutically acceptable salts thereof, are useful for therapeutic administration to enhance at least one of activation, priming, migration, proliferation, survival and cytolytic activity of T cells relative to prior to administration. In certain aspects, T cell activation is characterized by enhanced levels of IL-2, IFN-gamma, or granzyme B production by T cells or surface markers relative to prior to administration of the compound or pharmaceutically acceptable salt thereof. In some instances, the compounds of the disclosure, or pharmaceutically acceptable salts thereof, are useful for therapeutic administration to enhance differentiation of T cells toward cytotoxic/exhausted phenotypes. In some instances, the compounds of the disclosure, or pharmaceutically acceptable salts thereof, are useful for modulating the levels of phosphorylation, stability, and activity of immune pathway transcription factors such as NFAT and c-Jun. In some instances, the compounds of the disclosure, or pharmaceutically acceptable salts thereof, are useful for modulating autophagy pathway in both tumor and immune cells.

In some instances, the compounds of the disclosure, or pharmaceutically acceptable salts thereof, are useful for decreasing recruitment, infiltration, and differentiation of monocytes, tumor-associated macrophage, and myeloid-derived suppressor cell. In some instances, the compounds of the disclosure, or pharmaceutically acceptable salts thereof, are useful for modulating regulatory T cells. In some instances, the compounds of the disclosure, or pharmaceutically acceptable salts thereof, are useful for enhancing cytotoxic function of NK cells. In some instances, the compounds of the disclosure, or pharmaceutically acceptable salts thereof, are useful for enhancing secretion of inflammatory cytokines by immune cell types other than T lymphocytes. In some instances, the compounds of the disclosure, or pharmaceutically acceptable salts thereof, indirectly inhibit the growth of cancer cells via modulation of the tumor microenvironment. In some instances, the compounds of the disclosure, or pharmaceutically acceptable salts thereof, are useful for priming of the immune response (i.e., vaccines) to tumors or viruses for boosting or generating anti-viral/anti-tumor immunity. In one instance, the compounds of the disclosure, or pharmaceutically acceptable salts thereof, are useful for enhancing or boosting response to a vaccine (such as a cancer vaccine or a personalized cancer vaccine (PCV)) or a CAR-T cell therapy.

Methods of treating a disease or disorder responsive to inhibition of GSK3 (e.g., GSK3α) activity can include administering to a patient in need thereof a therapeutically effective amount of a compound provided herein, or a pharmaceutically acceptable salt thereof. For example, the disease or disorder is a cancer. The term “cancer” encompasses all forms of cancer including, but not limited to, all forms of carcinoma, melanomas, blastomas, sarcomas, lymphomas, leukemias. In some embodiments, cancer includes metastatic forms. Additionally, the disclosure includes refractory or recurrent malignancies whose growth may be inhibited using the compounds of the disclosure or pharmaceutically acceptable salts thereof. For the uses described herein, any of the compounds of the disclosure, or pharmaceutically acceptable salts thereof, may be used alone or in combination with other therapeutic agents.

In some embodiments, the treatment results in a sustained response in the subject after cessation of the treatment. “Sustained response” refers to the sustained effect on reducing tumor growth after cessation of a treatment. For example, the tumor size may remain the same or smaller as compared to the size at the beginning of the administration phase. In some embodiments, the sustained response has a duration at least the same as the treatment duration, at least 1.5×, 2.0×, 2.5×, or 3.0× length of the treatment duration.

The treatment methods disclosed herein may result in a partial or complete response. As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started. As used herein, “overall response rate” (ORR) refers to the sum of complete response (CR) rate and partial response (PR) rate.

The treatment methods disclosed herein can lead to an increase in progression free survival and overall survival of the subject administered the selective GSK3α inhibitor. As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall survival” (OS) refers to the percentage of subjects in a group who are likely to be alive after a particular duration of time.

Other cancers include, for example, sweat gland cancer, spinal axis tumor, chest cancer, and environmentally induced cancers including those induced by asbestos. In some embodiments, the cancer that are treatable using the compounds of the disclosure or pharmaceutically acceptable salts thereof, include, but are not limited to, an advanced solid tumor with an inflamed phenotype. In some embodiments, an inflamed solid tumor is a tumor mutational burden-high (TMB-H) cancer. In some embodiments, the cancer is selected from the group consisting of bladder cancer (including urothelial carcinoma and Bacille Calmette-Guérin (BCG)-unresponsive high-risk nonmuscle invasive bladder cancer (NMIBC)), breast cancer (including triple-negative breast cancer), cervical cancer, colorectal cancer, endometrial carcinoma, cutaneous squamous cell carcinoma, gastroesophageal cancer (including gastric carcinoma, gastro-oesophageal junction cancer, and oesophageal squamous cell carcinoma), hepatocellular carcinoma, leukemia (including acute myeloid leukemia (AML)), lung cancer (including malignant pleural mesothelioma, non-small cell lung cancer (NSCLC), and small cell lung cancer (SCLC)), melanoma (including metastatic melanoma), lymphoma (including Hodgkin's lymphoma, non-Hodgkin lymphoma (NHL, including diffuse large B cell lymphoma (DLBCL)), and primary mediastinal large B-cell lymphoma), Merkel cell carcinoma, microsatellite instability-high (MSI-H) cancer, mismatch repair deficiency (dMMR) cancer, ovarian cancer, pancreatic cancer, prostate cancer, renal cell carcinoma, squamous cell carcinoma of the head and neck (SCCHN), tumor mutational burden-high (TMB-H) cancer, and uterine cancer. In some embodiments, the cancer is selected from the group consisting of AML, bladder cancer, breast cancer, colorectal cancer, endometrial carcinoma, gastroesophageal cancer, hepatocellular carcinoma, melanoma, NHL, NSCLC, ovarian cancer, renal cell carcinoma, SCLC, and SCCHN. In some embodiments, the cancer is selected from the group consisting of bladder cancer, breast cancer, gastroesophageal cancer, hepatocellular carcinoma, malignant melanoma, NSCLC, renal cell carcinoma, and SCCHN. In some embodiments, the cancer is selected from the group consisting of hepatocellular carcinoma, melanoma, NSCLC, SCLC, and urothelial carcinoma. In some embodiments, the cancer is selected from the group consisting of melanoma, NSCLC, and urothelial carcinoma.

In some embodiments, diseases and disorders that are treatable using the compounds of the disclosure or pharmaceutically acceptable salts thereof include, but are not limited to hematological cancer, sarcomas, respiratory cancer, gastrointestinal cancer, genitourinary tract cancer, liver cancer, bone cancer, nervous system cancer, gynecological cancer, and skin cancer.

Exemplary hematological cancer includes, for example, lymphomas and leukemias such as acute lymphotic leukemia (ALL), AML, acute promyelocyte leukemia (APL), chronic lymphotic leukemia (CLL), chronic myeloid leukemia (CML), DLBCL, mantle cell lymphoma, Non-Hodgkin lymphoma (NHL), including Primary mediastinal B-cell lymphoma (PMBCL), relapsed or refractory NHL, recurrent follicular, and primary CNS lymphoma, Hodgkin's lymphoma, myeloproliferative diseases, including, primary myelofibrosis (PMF), polycythemia vera (PV), essential thrombocytosis (ET), myelodysplasia syndrome (MDS), T-cell acute lymphoblastic lymphoma (T-ALL), multiple myeloma, cutaneous T-cell lymphoma, Waldenstrom's Macroglubulinemia, hairy cell lymphoma, chronic myelogenic lymphoma, and Burkitt's lymphoma.

Exemplary sarcoma includes, for example, chondrosarcoma, Ewing's sarcoma, Kaposi's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, sarcoma of the soft tissue, and teratoma.

Exemplary respiratory tract cancer includes, for example, lung cancer such as non-small cell lung cancer (NSCLC), small cell lung cancer, epidermoid cancer, bronchogenic carcinoma, including squamous cell, undifferentiated small cell, undifferentiated large cell, adenocarcinoma, alveolar (bronchiolar) carcinoma, bronchial adenoma, chondromatous hamartoma, mesothelioma, and pleuropulmonary blastoma.

Exemplary gastrointestinal cancer includes, for example, cancers of the esophagus, including squamous cell carcinoma, adenocarcinoma, leiomyosarcoma, and lymphoma; stomach, including carcinoma, lymphoma, and leiomyosarcoma; pancreas, including ductal adenocarcinoma, insulinoma, glucagonoma, gastrinoma, carcinoid tumors, and vipoma; small instestine, including adenocarcinoma, lymphoma, carcinoid tumors, Kaposi's sarcoma, leiomyoma, hemangioma, lipoma, neurofibroma, and fibroma; large intestine, including adenocarcinoma, tubular adenoma, villous adenoma, hamartoma, and leiomyoma; colon; and gall bladder, including adenocarcinoma; and intestinal type and diffuse type gastric adenocarcinoma, rectum carcinoma, familiar adenomatous polyposis carcinoma and hereditary non-polyposis colorectal cancer (CRC).

Exemplary genitourinary tract cancer includes, for example, cancers of the kidney, including adenocarcinoma, Wilm's tumor [nephroblastoma], renal cell carcinoma, urothelial carcinoma, juxtaglomerular cell tumor (reninoma), angiomyolipoma, renal oncocytoma, Bellinio duct carcinoma, clear-cell sarcoma of the kidney, and mesoblastic nephroma; adrenal gland; renal pelvis; bladder, including transitional cell carcinoma, squamous cell carcinoma, adenocarcinoma, sarcoma, and small cell carcinoma; urethra, including squamous cell carcinoma, transitional cell carcinoma, and adenocarcinoma; prostate, including adenocarcinoma, sarcoma, and carcinoma; testis, including seminoma, teratoma, embryonal carcinoma, teratocarcinoma, choriocarcinoma, sarcoma, interstitial cell carcinoma, fibroma, fibroadenoma, adenomatoid tumors, and lipoma; penis; and pancreas.

Exemplary liver cancer includes, for example, hepatoma, including hepatocellular carcinoma, cholangiocarcinoma, hepatoblastoma, angiosarcoma, hepatocellular adenoma, biliary tract cancer, and hemangioma.

Exemplary bone cancer includes, for example, osteogenic sarcoma, fibrosarcoma, malignant fibrous histiocytoma, chondrosarcoma, Ewing's sarcoma, malignant lymphoma, including reticulum cell sarcoma, multiple myeloma, malignant giant cell tumor chordoma, osteochronfroma, including osteocartilaginous exostoses, benign chondroma, chondroblastoma, chondromyxofibroma, osteoid osteoma, and giant cell tumors

Exemplary nervous system cancer includes, for example, cancer of the skull, including osteoma, hemangioma, granuloma, xanthoma, and osteitis deformans; meninges including, meningioma, meningiosarcoma, and gliomatosis; brain, including astrocytoma, meduoblastoma, glioma, ependymoma, germinoma (pinealoma), neuroectodermal tumor, glioblastoma, glioblastoma multiform, oligodendroglioma, schwannoma, retinoblastoma, congenital tumors, brain stem and hypopthamic glioma; and spinal cord, including neurofibroma, meningioma, glioma, and sarcoma; as well as neuroblastoma and Lhermitte-Duclos disease.

Exemplary gynecological cancer includes, for example, cancer of the uterus, including endometrial carcinoma; cervix, including cervical carcinoma, pre-tumor cervical dysplasia, squamouse cell carcinoma, adenocarcinoma, adenosquamous carcinoma, small cell carcinoma, neuroendocrine tumor, glassy cell carcinoma and villoglandular adenocarcinoma; ovaries, including ovarian carcinoma (serous cystadenocarcinoma, mucinous cystadenocarcinoma, unclassified carcinoma, endometroid tumor, high-grade serous carcinoma (HGSC) or high-grade serous ovarian cancer (HGSOC)), granulosa-thecal cell tumors, Sertoli-Leydig cell tumors, dysgerminoma, malignant teratoma, and arrhenoblastoma; vulva (squamous cell carcinoma, intraepithelial carcinoma, adenocarcinoma, fibrosarcoma, and melanoma; vagina, including clear cell carcinoma, squamous cell carcinoma, and botryoid sarcoma (embryonal rhabdomyosarcoma); labia; and fallopian tubes.

Exemplary skin cancer includes, for example, melanoma, sebaceous gland carcinoma, basal cell carcinoma, squamous cell carcinoma, Kaposi's sarcoma, Merkel cell skin cancer, moles dysplastic nevi, lipoma, angioma, dermatofibroma, and keloids.

Examples of breast cancer include, for example, ER+/HER2-breast cancer, triple-negative breast cancer (TNBC), invasive ductal carcinoma, invasive lobular carcinoma, ductal carcinoma in situ, and lobular carcinoma in situ.

Exemplary head and neck cancer includes, for example, glioblastoma, melanoma, rhabdosarcoma, lymphosarcoma, osteosarcoma, squamous cell carcinomas, adenocarcinomas, oral cancer, throat cancer, including oropharyngeal cancer, laryngeal cancer, nasopharyngeal cancer, nasal and paranasal cancer, salivary gland cancer, mouth cancer, eye cancer, acoustic neuroma, pituitary adenoma, hypopharynx, and thyroid (medullary and papillary) and parathyroid cancer.

Other cancers include, for example, sweat gland cancer, spinal axis tumor, chest cancer, and environmentally induced cancers including those induced by asbestos.

In some instances, the disease or disorder that are treatable using the compounds of the disclosure or pharmaceutically acceptable salts thereof is a viral infection, such as infection caused by hepatitis B virus (HBV), hepatitis C virus (HCV), human papilloma virus (HPV), cytomegalovirus (CMV), herpes simplex virus (HSV), Epstein-Barr virus (EBV), varicella zoster virus, coxsackie virus, and human immunodeficiency virus (HIV).

### Combination Therapies

Compounds of the disclosure or pharmaceutically acceptable salts thereof, can be administered as the sole pharmaceutical agent or in combination with one or more other anti-cancer agents for the treatment of cancer, where the combination causes no unacceptable adverse effects. In some embodiments, the other anti-cancer agents are immune-oncology agent, anticancer agents that are enzyme/protein/receptor inhibitors, radiation or chemotherapy.

Compounds of the disclosure or pharmaceutically acceptable salts thereof, can be co-formulated with an immuno-oncology agent. Immuno-oncology agents include, for example, a small molecule drug, antibody, or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human. In another aspect, the antibody is a bispecific antibody.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fnl4, TWEAK, BAFFR, EDAR, XEDAR, TACI, APRIL, BCMA, LTfiR, LIGHT, DcR3, HVEM, VEGI/TLIA, TRAMP/DR3, EDAR, EDA1, XEDAR, EDA2, TNFR1, Lymphotoxin a/TNPB, TNFR2, TNF a, LT R, Lymphotoxin a 182, FAS, FASL, RELT, DR6, TROY, NGFR.

In one aspect, T cell responses can be stimulated by a combination of a compound of the disclosure or a pharmaceutically acceptable salt thereof, and one or more of (i) an antagonist of a protein that inhibits T cell activation (e.g., immune checkpoint inhibitors) such as CTLA-4, PD-1, PD-L1, PD-L2, LAG-3, TIM−3, Galectin 9, CEACAM−1, BTLA, CD69, Galectin-1, TIGIT, CD113, GPR56, VISTA, 2B4, CD48, GARP, PD1H, LAIR1, TIM−1, and TIM−4, and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In one aspect, compounds of the disclosure or pharmaceutically acceptable salts thereof can be administered in combination with at least one other immune checkpoint inhibitor. In other aspects, compounds of the disclosure or pharmaceutically acceptable salts thereof can be administered for the treatment of immune checkpoint inhibitor-resistant NSCLC, including where the subject is refractory to, or had a partial response to, platinium and/or paclitaxel and/or docetaxel therapy. Optionally, compounds of the disclosure or pharmaceutically acceptable salts thereof can be administered in combination with at least one other anti-cancer agent, such as paclitaxel, docetaxel or platinum anticancer therapy. In some aspects, compounds of the disclosure or pharmaceutically acceptable salts thereof can be administered post-platinium therapy as second or third line treatment. Compounds of the disclosure or pharmaceutically acceptable salts thereof can be administered for the treatment of first line NSCLC expressing high PD-L1 (>50% Tumor Proportion Score (TPS)), wild-type EGFR, or wild-type ALK.

Other agents that can be combined with compounds of the disclosure or pharmaceutically acceptable salts thereof, for the treatment of cancer include antagonists of inhibitory receptors on NK cells or agonists of activating receptors on NK cells. For example, compounds of the disclosure can be combined with antagonists of KIR, such as lirilumab.

Yet other agents for combination therapies include agents that inhibit or deplete macrophages or monocytes, including but not limited to CSF-1R antagonists such as CSF-1R antagonist antibodies including RG7155 or FPA-008.

In another aspect, compounds of the disclosure or pharmaceutically acceptable salts thereof, can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In some embodiments, the immuno-oncology agent is a CTLA-4 antagonist, such as an antagonistic CTLA-4 antibody. Suitable CTLA-4 antibodies include, for example, YERVOY (ipilimumab) or tremelimumab. In another aspect, the immuno-oncology agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgGI, called AMP-224

In another aspect, the immuno-oncology agent is a PD-L1 antagonist, such as an antagonistic PD-L1 antibody. Suitable PD-L1 antibodies include, for example, TECENTRIQ (atezolizumab) (RG7446; WO2010/077634), durvalumab (MEDI4736), BMS-936559 (WO2007/005874), and MSB0010718C (WO2013/79174).

In another aspect, the immuno-oncology agent is a LAG-3 antagonist, such as an antagonistic LAG-3 antibody. Suitable LAG3 antibodies include, for example, BMS-986016 (WO10/19570, WO14/08218), or IMP-731 or IMP-321 (WO08/132601, WO09/44273).

In another aspect, the immuno-oncology agent is a CD137 (4-1BB) agonist, such as an agonistic CD137 antibody. Suitable CD137 antibodies include, for example, urelumab and PF-05082566 (WO12/32433).

In another aspect, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WO1 1/028683).

In another aspect, the immuno-oncology agent is an IDO antagonist. Suitable IDO antagonists include, for example, INCB-024360 (WO2006/122150, WO07/75598, WO08/36653, WO08/36642), indoximod, or NLG-919 (WO09/73620, WO09/1156652, WO111/56652, WO12/142237).

In another aspect, the immuno-oncology agent is an OX40 agonist, such as an agonistic OX40 antibody. Suitable OX40 antibodies include, for example, MEDI-6383 or MEDI-6469. In another aspect, the immuno-oncology agent is an OX40L antagonist, such as an antagonistic OX40 antibody. Suitable OX40L antagonists include, for example, RG-7888 (WO06/029879).

In another aspect, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab.

In another aspect, the immuno-oncology agent is a CD27 agonist, such as an agonistic CD27 antibody. Suitable CD27 antibodies include, for example, varlilumab.

In another aspect, the immuno-oncology agent is MGA271 (to B7H3) (WO1 1/109400).

The compounds of the disclosure or pharmaceutically acceptable salts thereof, can be used in combination with anticancer agents that are enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

The compounds of the disclosure or pharmaceutically acceptable salts thereof, can be used in combination with one or more other enzyme/protein/receptor inhibitors for the treatment of cancer. For example, the compounds of the disclosure or pharmaceutically acceptable salts thereof, can be combined with one or more inhibitors of the following kinases for the treatment of cancer: Akt1, Akt2, Akt3, TGF-βPv, PKA, PKG, PKC, CaM-kinase, phosphorylase kinase, MEKK, ERK, MAPK, mTOR, EGFR, HER2, HER3, HER4, INS—R, IGF-1R, IR—R, PDGFotR, PDGFpR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK, and B-Raf.

In some embodiments, the compounds of the disclosure or pharmaceutically acceptable salts thereof, can be combined with one or more of the following inhibitors for the treatment of cancer. Non-limiting examples of inhibitors that can be combined with the compounds of the present disclosure for treatment of cancers include an FGFR inhibitor (FGFR1, FGFR2, FGFR3 or FGFR4, e.g., fisogatinib, AZD4547, BAY 1187982, ARQ087, BGJ398, BIBF1120, TKI258, lucitanib, dovitinib, TAS-120, J J-42756493, Debio1347, INCB54828, INCB62079, and INCB63904), a JAK inhibitor (JAK1 and/or JAK2, e.g., ruxolitinib, baricitinib, or itacitinib (INCB39110)), an IDO inhibitor (e.g., epacadostat and NLG919), an LSD1 inhibitor (e.g., GSK2979552, INCB59872 and INCB60003), a TDO inhibitor, a PI3K-delta inhibitor (e.g., INCB50797 and INCB50465), a PI3K-gamma inhibitor such as a PI3K-gamma selective inhibitor (eganelisib) or a dual PI3K-delta/gamma selective inhibitor (duvelisib), a CSFIR inhibitor (e.g., PLX3397 and LY3022855), a TAM receptor tyrosine kinases (Tyro-3, Axl, and Mer), an angiogenesis inhibitor (Such as Avastin (bevacizumab)), an interleukin receptor inhibitor, bromo and extra terminal family members inhibitors (for example, bromodomain inhibitors or BET inhibitors such as OTX015, CPI-0610, INCB54329, and INCB57643), and an adenosine receptor antagonist or combinations thereof. Inhibitors of HDAC such as panobinostat and vorinostat can be combined with the compounds of the disclosure. Inhibitors of c-Met such as onartumzumab, tivantnib, and capmatinib (INC-280) be combined with the compounds of the disclosure or pharmaceutically acceptable salts thereof. Inhibitors of BTK such as ibrutinib can be combined with the compounds of the disclosure or pharmaceutically acceptable salts thereof. Inhibitors of mTOR such as rapamycin, sirolimus, temsirolimus, and everolimus can be combined with the compounds of the disclosure or pharmaceutically acceptable salts thereof. Inhibitors of Raf, such as vemurafenib and dabrafenib can be combined with the compounds of the disclosure or pharmaceutically acceptable salts thereof. Inhibitors of MEK such as trametinib, selumetinib and GDC-0973 can be combined with the compounds of the disclosure or pharmaceutically acceptable salts thereof. Inhibitors of KIT, including avapritinib, imatinib, sunitinib, regorafenib, ripritinib (DCC2618), PLX9486, PLX3397, crenolanib, CDX-0158, CDX-0159. Inhibitors of RET including pralsetinib, selperctinib, alectinib, levatinib, cabozantinib, BOS172738 (DS-5010), SL-1001, TPX-0046, sitravatinib (MGCD516), and RXDX-105. Inhibitors of Hsp90 (e.g., tanespimycin), cyclin dependent kinases (e.g., palbociclib), PARP (e.g., olaparib) and Pim kinases (LGH447, INCB053914, and SGI-1776), and KRAS (e.g., sotorasib) can also be combined with compounds of the disclosure or pharmaceutically acceptable salts thereof.

Compounds of the disclosure or pharmaceutically acceptable salts thereof, can be used in combination with one or more agents for the treatment of cancer. In some embodiments, the agent is an alkylating agent, a proteasome inhibitor, a corticosteroid, or an immunomodulatory agent. Examples of an alkylating agent include bendamustine, nitrogen mustards, ethylenimine derivatives, alkyl sulfonates, nitrosoureas and triazenes, uracil mustard, chlormethine, cyclophosphamide (CYTOXAN), ifosfamide, melphalan, chlorambucil, pipobroman, triethylene-melamine, triethylenethiophosphoramine, busulfan, carmustine, lomustine, streptozocin, dacarbazine, and temozolomide. In some embodiments, the proteasome inhibitor is carfilzomib. In some embodiments, the corticosteroid is dexamethasone (DEX).

The compounds of the disclosure or pharmaceutically acceptable salts thereof, can be administered in combination with one or more anti-cancer drugs, such as a chemotherapeutics. Example chemotherapeutics include any of: abarelix, abiraterone, afatinib, aflibercept, aldesleukin, alemtuzumab, alitretinoin, allopurinol, altretamine, anastrozole, arsenic trioxide, asparaginase, axitinib, azacitidine, bevacizumab, bexarotene, baricitinib, bicalutamide, bleomycin, bortezombi, bortezomib, brivanib, buparlisib, busulfan intravenous, busulfan oral, calusterone, capecitabine, carmustine, cediranib, cetuximab, chlorambucil, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, docetaxel, doxorubicin, droloxafine, dromostanolone propionate, eculizumab, enzalutamide, epidophyllotoxin, epirubicin, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, nofetumomab, oserelin, paclitaxel, pamidronate, panitumumab, pazopanib, pegaspargase, pegfilgrastim, pemetrexed disodium, pentostatin, pilaralisib, pipobroman, plicamycin, cisplatin, carboplatin, oxaliplatin, ponatinib, prednisone, procarbazine, quinacrine, rasburicase, regorafenib, reloxafine, rituximab, ruxolitinib, sorafenib, streptozocin, sunitinib, sunitinib maleate, tamoxifen, tegafur, temozolomide, teniposide, testolactone, thalidomide, thioguanine, thiotepa, topotecan, toremifene, tositumomab, trastuzumab, tretinoin, triptorelin, uracil mustard, valrubicin, vandetanib, vinblastine, vincristine, vinorelbine, vorinostat, and zoledronate.

Other anti-cancer agent(s) include antibody therapeutics such as trastuzumab (Herceptin).

Compounds of the disclosure or pharmaceutically acceptable salts thereof, can be administered as the sole pharmaceutical agent or in combination with one or more anti-viral agents for the treatment of chronic viral infections, where the combination causes no unacceptable adverse effects. Chronic viral infections include, but are not limited to, diseases caused by: hepatitis C virus (HCV), human papilloma virus (HPV), cytomegalovirus (CMV), herpes simplex virus (HSV), Epstein-Barr virus (EBV), varicella zoster virus, coxsackie virus, human immunodeficiency virus (HIV). Parasitic infections (e.g., malaria) may also be treated by the above methods wherein compounds known to treat the parasitic conditions are optionally added in place of the antiviral agents.

Suitable antiviral agents contemplated for use in combination with the compounds of the disclosure or pharmaceutically acceptable salts thereof, can comprise nucleoside and nucleotide reverse transcriptase inhibitors (NRTIs), non-nucleoside reverse transcriptase inhibitors (NNRTIs), protease inhibitors and other antiviral drugs.

Examples of suitable NRTIs include zidovudine (AZT); didanosine (ddl); zalcitabine (ddC); stavudine (d4T); lamivudine (3TC); abacavir (1592U89); adefovir dipivoxil [bis (POM)-PMEA]; lobucavir (BMS-180194); BCH-10652; emitricitabine [(−)-FTC]; beta-L-FD4 (also called beta-L-D4C and named beta-L-2′,3′-dicleoxy-5-fluoro-cytidene); DAPD, ((−)-beta-D-2,6-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrirnidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1549. Other antiviral agents include hydroxyurea, ribavirin, IL-2, IL-12, pentafuside and Yissum Project No. 11607.

When more than one pharmaceutical agent is administered to a patient, they can be administered simultaneously, separately, sequentially, or in combination (e.g., for more than two agents). For examples, when administered together with an additional anti-cancer or antiviral agent, the disclosed compounds or pharmaceutically acceptable salts thereof, can be administered simultaneously in the same pharmaceutical formulation or simultaneously in separate pharmaceutical formulations. Alternatively, when administered together with an additional anti-cancer or antiviral agent, the disclosed compounds or pharmaceutically acceptable salts thereof, can be administered at separate times, depending the dosing requirements of the additional anti-cancer or antiviral agent.

Pharmaceutical compositions are disclosed that include one or more compounds provided herein (such as the compound of any one of Formulae (I′), (I), (II), (IIa), (IIb), (IIc), (III), (IIIa), (IIIb), (IV), (IVa), (IVb), (IVc), (IVd), (V), (Va), (Vb), (VI), (VIa), (VIb), (VIc), (VId), (VIe), (VIf), (VIg), (VIh), (VII), (VIIa), (VIIb), (VIIIa), (VIIIb), (VIIIc), (VIIId), (VIIIe), (VIIIf), (VIIIg), (VIIIh), (IXa), (IXb), and (X), or a compound as disclosed in the Exemplification) or pharmaceutically acceptable salts thereof, and typically at least one additional substance, such as an excipient, a known therapeutic other than those of the disclosure, and combinations thereof. In some embodiments, the disclosed compounds or pharmaceutically acceptable salts thereof, can be used in combination with other agents known to have beneficial activity targeting diseases or disorders listed above. For example, disclosed compounds or pharmaceutically acceptable salts thereof, can be administered alone or in combination with one or more anti-cancer or antiviral agent, and the pharmaceutically acceptable salts of these compounds.

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

A “subject” is a mammal in need of medical treatment, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The precise amount of compound or pharmaceutically acceptable salt thereof, administered to provide an “effective amount” to the subject will depend on the mode of administration, the type, and severity of the disease or condition, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer or antiviral agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the disclosure or pharmaceutically acceptable salt thereof, being used by following, for example, dosages reported in the literature and recommended in the Physician's Desk Reference (57th ed., 2003).

The term “effective amount” means an amount when administered to the subject which results in beneficial or desired results, including clinical results, e.g., inhibits, suppresses or reduces the symptoms of the condition being treated in the subject as compared to a control.

The particular mode of administration and the dosage regimen will be selected by the attending clinician, taking into account the particulars of the case (e.g. the subject, the disease, the disease state involved, the particular treatment, and whether the treatment is prophylactic). Treatment can involve daily or multi-daily or less than daily (such as weekly or monthly etc.) doses over a period of a few days to months, or even years.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

“Pharmaceutically acceptable excipient” and “pharmaceutically acceptable carrier” refer to a substance that aids the formulation and/or administration of an active agent to and/or absorption by a subject and can be included in the compositions of the present disclosure without causing a significant adverse toxicological effect on the subject. Non limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

### General Synthetic Methods and Intermediates

Compounds of the disclosure, including salts and N-oxides thereof, can be prepared using organic synthesis techniques known to one of ordinary skill in the art and/or by reference to the schemes shown below and the synthetic examples. The below Schemes are synthetic protocols that are meant to provide general guidance in connection with preparing the compounds of the disclosure. One skilled in the art would understand that the preparations shown in the Schemes can be modified or optimized using general knowledge of organic chemistry to prepare various compounds of the disclosure.

The reactions for preparing compounds of the disclosure can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the disclosure can involve the protection and deprotection of various chemical groups. The need for protection and deprotection, and the selection of appropriate protecting groups, can be readily determined by one skilled in the art. The chemistry of protecting groups can be found, for example, in Wuts and Greene, Protective Groups in Organic Synthesis, 5th ed., John Wiley & Sons: New Jersey, (2014), which is incorporated herein by reference in its entirety.

Reactions can be monitored according to any suitable method known in the art. For example, product formation can be monitored by spectroscopic means, such as nuclear magnetic resonance (NMR) spectroscopy (e.g., 1H or 13C), infrared (IR) spectroscopy, spectrophotometry (e.g., UV-visible), mass spectrometry (MS), or by chromatographic methods such as high performance liquid chromatography (HPLC) or thin layer chromatography (TLC).

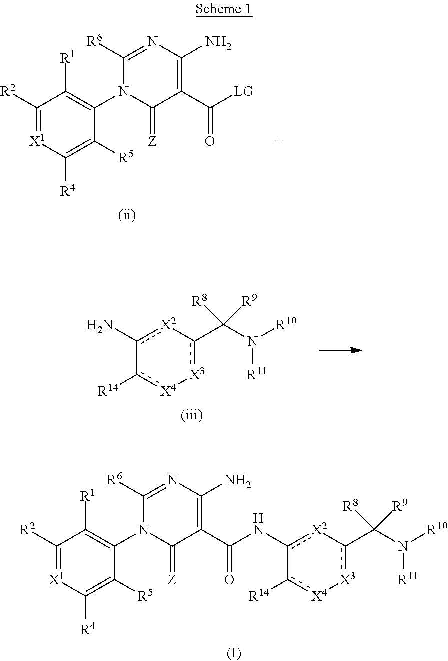

LG is a leaving group, typically OMe

In Scheme 1, a compound of Formula (I) may be prepared from compounds of Formula (ii) and (iii) using an amide coupling reaction typically in the presence of Me₃Al in an aprotic solvent such as toluene at elevated temperature. A protecting group such a tBoc may be removed in situ or as a separate step using a suitable acid such as TFA or HCl.

Compounds of Formula (I) may undergo further reaction to provide an alternative compound of Formula (I). For example, when X¹=CR³ where R³=Halo, single or multiple functional group interconversions as known by those skilled in the art, may be carried out to obtain alternative compounds where R³ is defined in Formula (I).

It will be appreciated by those skilled in the art that it may be necessary to utilise a suitable protecting group strategy for the preparation of compounds of Formula (I). Typical protecting groups may comprise, 4-methoxybenzyl, 2,4-dimethoxybenzyl, benzyl, carbamate or phthalimide, preferably Boc or phthalimide for the protection of primary or secondary aliphatic amines.

Also, it will be further appreciated that it may be necessary and/or desirable to carry out the transformations in a different order from that described in the schemes, or to modify one or more of the transformations, to provide the desired compound of the invention.

In particular, typical functional group interconversions (FGIs) include, where X¹=CR³ where CR³ is =CHal, conversion into other compounds of Formula (I) where X¹=CR³ where R³ is defined in Formula (I); a) palladium catalysed cross-coupling with a heterocyclic boronic acid or boronate; b) conversion of CHal to a boronic acid followed by palladium catalysed cross-coupling; c) reaction of CHal with an appropriate alcohol to afford the corresponding ethers; d) conversion of CHal into alternative compounds where R³ is described in Formula (I).

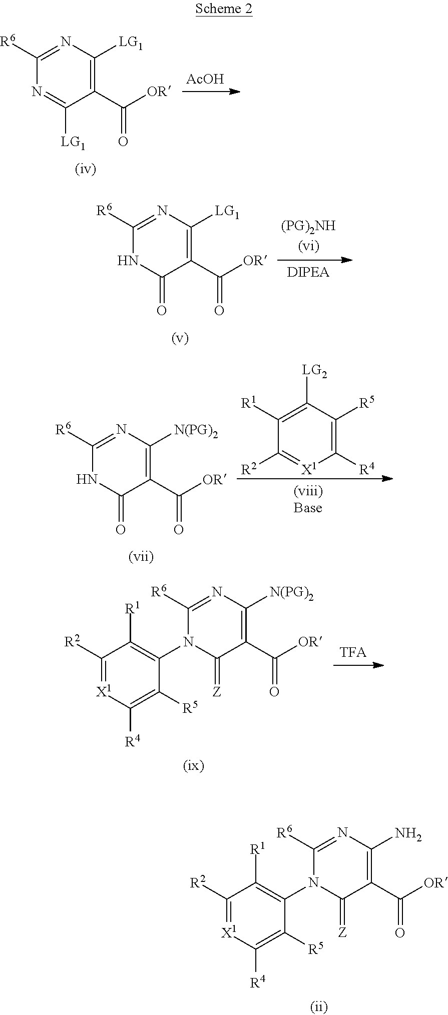

LG1 a leaving group, typically a C₁. OR′ is an alkyloxy group, typically OMe.

PG is an amine protecting group, typically 4-methoxybenzyl, 2,4-dimethoxylbenzyl and Boc

In Scheme 2, compounds of Formula (ii) may be typically prepared from compounds of Formulae (iv), (v), (vi), (vii), (viii) and (ix) using the steps outlined.

Compounds of Formula (v) may prepared by the hydrolysis of compounds of Formula (iv) in AcOH at elevated temperatures.

Compounds of Formula (vii) may be prepared by reaction of compounds of Formula (v) with an appropriate amine (PG)₂NH of Formula (vi) in the presence of an organic base such as DIPEA in an aprotic solvent such as DMSO at elevated temperature.

Compounds of Formula (ix) may be prepared by reaction of compounds of Formulae (vii) and (viii) in the presence of a base such as K₂CO₃ or DIPEA in DMF or DMSO at elevated temperatures.

Compounds of Formula (ii) may be prepared by deprotection of compounds of Formula (ix) in excess TFA at elevated temperatures.

Compounds of Formula (ii) and/or (ix) may undergo further reaction to provide an alternative compound of Formula (I). For example, when X¹=CR³ where R³=Halo, single or multiple functional group interconversions may be carried out to obtain alternative compounds where R³ is defined in Formula (I).

It will be appreciated that it may be necessary and/or desirable to carry out the transformations in a different order from that described in the schemes, or to modify one or more of the transformations, to provide the desired compound of the invention.

Compounds that contain one or more stereocenters may be separated into their separate stereoisomers by typical methods such as chiral SFC or chiral HPLC techniques as indicated in the Intermediates and Examples below.

The following examples are intended to be illustrative, and are not meant in any way to be limiting.

### EXEMPLIFICATION

### Abbreviations

9-BBN 9-borabicyclo[3.3.1]nonane
Å angstrom
Ac₂O acetic anhydride
AcOH acetic acid
ATP adenosine-5′-triphosphate
BINAP 2,2′-bis(diphenylphosphino)-1,1′-binaphthyl
BID twice a day dosing
BIW twice a week dosing
Boc tert-butyloxycarbonyl
(Boc)₂O di-tert-butyl dicarbonate
BOP (1H-Benzotriazol-1-yloxy) [tris(dimethylamino)]phosphonium hexafluorophosphate
C Celsius
CSA camphorsulfonic acid
DAST Diethylaminosulfur trifluoride
DCE dichloroethane
DCM dichloromethane
DEAD diethyl azodicarboxylate
DIPA N,N-diisopropylamine
DIPEA N,N-diisopropylethylamine
DME dimethoxyethane
DMF dimethyl formamide
DMAP 4-Dimethylaminopyridine
DMSO dimethylsulfoxide
DPPA diphenylphosphoryl azide
DTT dithiothreitol
EDC-HCl (3-dimethylaminopropyl)-ethyl-carbodiimide hydrochloride
EDCI 1-ethyl-3-(3-dimethylaminopropyl) carbodiimide
EDTA ethylenediaminetetraacetic acid
EtOAc ethyl acetate
EtMgBr ethylmagnesium bromide
EtOH ethanol
h hour(s)
HATU 1-[bis(dimethylamino)methylene]-1H-1,2,3-triazolo[4,5-b]pyridinium 3-oxide hexafluorophosphate
HEPES N-2-hydroxyethylpiperazine-N′-2-ethanesulfonic acid
Hex hexane
HOBt 1-hydroxybenzotriazole
HP-β-CD 2-hydroxypropyl-β-cyclodextrin
HPLC high performance liquid chromatography
IC₅₀ inhibitory concentration 50%
IPA or i-PrOH isopropyl alcohol
Ir[dF(CF₃)ppy]₂(dtbpy))PF₆ [4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium (III) hexafluorophosphate
ip intraperitoneal
iv intravenous
KHMDS potassium hexamethyldisilazide or potassium bis(trimethylsilyl)amide
KOAc potassium acetate
LCMS liquid chromatography mass spectrometry
LDA lithium diisopropylamide
LiHMDS lithium bis(trimethylsilyl)amide
m/z mass to charge ratio
min minutes
MTBE methyl tert-butyl ether
MeCN acetonitrile
MeOH methanol
MPLC medium pressure liquid chromatography
NaOAc sodium acetate
NaOtBu sodium tert-butoxide
n-BuLi n-butyllithium
NMO N-methylmorpholine N-oxide
NMP N-Methyl-2-pyrrolidone
PBS phosphate-buffered saline
PdCl₂(PPh₃)₂ bis(triphenylphosphine) palladium(II) dichloride
Pd₂(dba)₃ tris(dibenzylideneacetone)palladium(0)
Pd(dppf)Cl₂ [1,1′-bis(diphenylphosphino) ferrocene]dichloropalladium (II)
Pd(OAc)₂ palladium (II) acetate
Pd(PPh₃)₄ Tetrakis(triphenylphosphine)palladium(0)
PE petroleum ether
PPh₃ triphenylphosphine
PPTS pyridinium p-toluenesulfonate
prep-HPLC preparative high performance liquid chromatography
prep-TLC preparative thin layer chromatography
rt room temperature
sat. aq. saturated aqueous
SFC supercritical fluid chromatography
t-BuOH tert-butyl alcohol
TBAB tetrabutylammonium bromide
TBAF tetrabutylammonium fluoride
TBHP tert-butyl hydroperoxide
TFA trifluoroacetic acid
TEA triethylamine
THF tetrahydrofuran
Ti(OⁱPr)₄ titanium (IV) isopropoxide or tetraisopropoxytitanium
TMSN₃ trimethylsilyl azide
HPLC Codes and Methods.
Organic gradient 0-100%, optimised for each sample.
HPLC-1 (Phenomenex Luna C18 75×30 mm, 3 μm; MeCN/H₂O (0.2% HCO₂H)); HPLC-2 (Phenomenex Luna C18; 150×30 mm, 5 μm; MeCN/H₂O (0.2% HCO₂H); HPLC-3 (Phenomenex Luna C18; 200×40 mm, 10 μm; MeCN/H₂O (0.2% HCO₂H); HPLC-4 (Phenomenex Luna C18; 100×30 mm, 5 μm; MeCN/H₂O (0.2% HCO₂H); HPLC-5 (Phenomenex Luna C18; 100×25 mm, 4 μm; MeCN/H₂O (0.2% HCO₂H); HPLC-6 (Phenomenex Luna C18; 100×40 mm, 3 μm; MeCN/H₂O (0.2% HCO₂H); HPLC-7 (Phenomenex Luna C18; 80×30 mm, 3 μm; MeCN/H₂O (0.2% HCO₂H); HPLC-8 (Phenomenex Luna C18; 75×30 mm, 3 μm; MeCN/H₂O (0.2% HCO₂H); HPLC-9 (Phenomenex Luna C18; 200×40 mm, 10 μm; MeCN/H₂O (0.2% HCO₂H); HPLC-10 (Gilson C18 150×20 μm; MeCN/H₂O (0.1% TFA); HPLC-11 (Waters Xbridge OBD 150×40 mm, 10 μm; MeCN/H₂O(NH₄HCO₃); HPLC-12 (Waters XBridge C18 150×19 mm, 3 μm; MeCN/H₂O (0.1% TFA); HPLC-13 (Waters XBridge BEH C18 100×30 mm, 10 μm; MeCN/H₂O (0.2% HCO₂H); HPLC-14 (Phenomenex Synergi Polar-RP, 100×25 mm, 4 μm; MeCN/H₂O (0.2% HCO₂H); HPLC-15 (Phenomenex Luna C18, 75×30 mm, 3 μm; MeCN/H₂O(NH₄HCO₃); HPLC-16 (Phenomenex Gemini NX C18, 75×30 mm, 3 μm; MeCN/H₂O (0.05% NH₄OH+10 mM NH₄HCO₃); HPLC-17 (Xtimate C18 250×80 mm, 10 μm; MeCN/H₂O (0.05% NH₄OH+10 mM NH₄HCO₃); HPLC-18 (YMC-Triart Prep C18, 250×50 mm, 10 μm; MeCN/H₂O (0.05% NH₄OH+10 mM NH₄HCO₃)); HPLC-19 (Agela DuraShell C18, 250×70 mm, 10 μm; MeCN/H₂O (0.05% NH₄OH+10 mM NH₄HCO₃)); HPLC-20 ((Phenomenex luna C18, 250×70 mm, 15 μm); 1-13% MeCN/H₂O (+HCl); HPLC-21 (Phenomenex Titank C18 Bulk 250×70 mm, 10 μm; MeCN/H₂O (0.05% NH₄OH+10 mM NH₄HCO₃)); HPLC-22 (Phenomenex Luna C18, 250×70 mm, 15 μm); MeCN/H₂O (0.2% HCO₂H)); HPLC-23 (Kromasil C18, 250×50 mm, 10 μm); MeCN/H₂O (0.05% NH₄OH+10 mM NH₄HCO₃)); HPLC-24 (Phenomenex Luna C18 75×30 mm, 3 μm; MeCN/(H₂O (+TFA)); HPLC-25 (Waters XBridge BEH C18 100×30 mm, 10 μm; MeCN/H₂O (0.05% NH₄OH+10 mM NH₄HCO₃); HPLC-26 (Welch Xtimate C18, 250×70 mm, 10 μm; MeCN/H₂O (0.05% NH₄OH+10 mM NH₄HCO₃)); HPLC-27 ((Waters Xbridge BEH C18, 250×50 mm, 10 μm; MeCN/H₂O (0.05% NH₄OH+10 mM NH₄HCO₃)).

### Intermediate A1. (S)-3-(1-amino-2-methoxyethyl)aniline

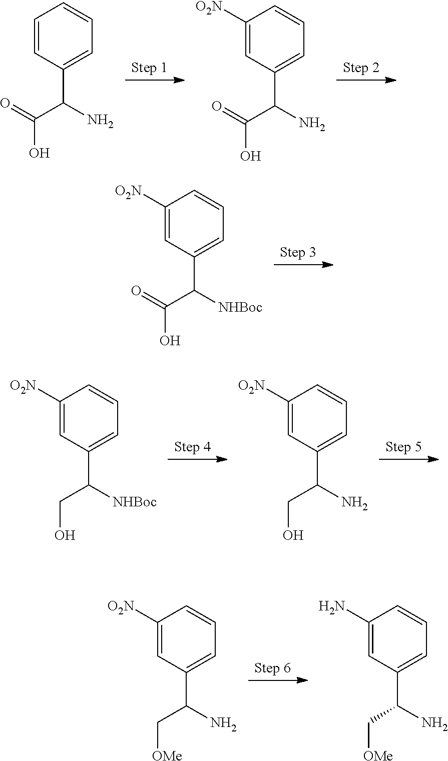

### Step 1. Synthesis of 2-amino-2-(3-nitrophenyl)acetic acid

To a solution of 2-amino-2-phenyl-acetic acid (20 g, 132 mmol) in H₂SO₄ (60 mL) was added the mixture of HNO₃ (24.9 mL) in H₂SO₄ (24 mL) at 0° C. and the mixture stirred at 0° C. for 3 h. The reaction was quenched by adding to iced H₂O (300 mL) and the solids collected by filtration. The solids were triturated with H₂O (200 mL) at 25° C. and stirred 20 min. Then solids were collected by filtration to afford the title compound as a pale yellow solid (25 g, 96%). LCMS m/z=197 [M+H]⁺.

### Step 2. Synthesis of 2-((tert-butoxycarbonyl)amino)-2-(3-nitrophenyl) acetic acid

To a solution of 2-amino-2-(3-nitrophenyl) acetic acid (25 g, 127 mmol) and (Boc)₂₀ (27.8 g, 127 mmol) in THF (50 mL) and H₂O (50 mL) was added Na₂CO₃ (27 g, 255 mmol) and the mixture stirred at 25° C. for 1 h. The reaction mixture was filtered and the filtrate evaporated to dryness in vacuo. The residue was triturated with HCl (20 mL) at 25° C. until pH=2 and stirred 20 min. The solids were collected by filtration to afford the title compound as a yellow solid (22 g, 58%) which was used without further purification.

### Step 3. Synthesis of tert-butyl(2-hydroxy-1-(3-nitrophenyl)ethyl)carbamate

To a solution of 2-(tert-butoxycarbonylamino)-2-(3-nitrophenyl) acetic acid (10.5 g, 35.4 mmol) in THF (50 mL) was added BH₃. THF (1 M, 70.9 mL) at 0° C. and the mixture stirred at 0° C. for 2 h. The reaction quenched by the addition of MeOH (200 mL) at 0° C. and concentrated under reduced pressure. The residue was purified by column chromatography (SiO₂, 0-50% EtOAc/PE) to give the title compound as a yellow oil (3.8 g, 38%). ¹H NMR (400 MHz, MeOH-d₄) δ: 8.28-8.23 (m, 1H), 8.16 (dd, 1H), 7.76 (d, 1H), 7.65-7.55 (m, 1H), 4.77 (s, 1H), 3.82-3.65 (m, 3H), 1.45 (s, 9H).

### Step 4. Synthesis of 2-amino-2-(3-nitrophenyl) ethan-1-ol

A solution of tert-butyl(2-hydroxy-1-(3-nitrophenyl)ethyl)carbamate (3.8 g, 13.5 mmol) in HCl/EtOAc (40 mL) was stirred at 25° C. for 1 h. The reaction mixture was concentrated under reduced pressure and the residue was purified by prep-HPLC (Xtimate C18, 250×80 mm, 10 μm; 1-25% MeCN/H₂O (0.05% NH₄OH+10 mM NH₄HCO₃)) to give the title compound as a white solid (1.83 g, 74%). LCMS m/z=183 [M+H]⁺.

### Step 5. Synthesis of 2-methoxy-1-(3-nitrophenyl) ethan-1-amine

To a solution of 2-amino-2-(3-nitrophenyl) ethanol (1 g, 5.49 mmol) in THF (10 mL) was added NaH (329 mg, 8.23 mmol) at 0° C. for 5 min, and MeI (779 mg, 5.49 mmol) added and the mixture stirred at 70° C. for 1 h. The reaction mixture was quenched by addition saturated aqueous NH₄Cl solution (15 mL) at 0° C. The pH was adjusted to pH=10 with sat·aq·Na₂CO₃ and then extracted with EtOAc (3×20 mL). The combined organics were dried (Na₂SO₄), evaporated to dryness in vacuo and the residue purified by MPLC (SiO₂, 0-16% MeOH/EtOAc) to give the title compound as a white solid (450 mg, 41.8%). LCMS m/z=197 [M+H]⁺.

### Step 6. Synthesis of (S)-3-(1-amino-2-methoxyethyl) aniline

To a solution of 2-methoxy-1-(3-nitrophenyl) ethan-1-amine (450 mg, 2.29 mmol) in MeOH (5 mL) was added Pd/C (450 mg, 2.29 mmol, 10% purity) and the mixture stirred at 25° C. for 1 h under H₂. The reaction mixture was filtered and the filtrate was concentrated under reduced pressure to give 3-(1-amino-2-methoxyethyl) aniline as a white solid (340 mg, 89%).

The residue was purified by prep-HPLC (Xtimate C18, 250×80 mm, 10 μm; 15-45% MeCN/H₂O(NH₄OH+NH₄HCO₃)) followed by SFC (REGIS (s,s) WHELK-01, 250×50 mm, 10 μm); 50% IPA (+0.1% NH₄OH) in CO₂) to afford:

Peak 1, Intermediate A1, (S)-3-(1-amino-2-methoxyethyl) aniline as a white solid (130 mg, 38%). LCMS m/z=167 [M+H]⁺.

### Intermediate A2. tert-butyl (R)-(1-(3-aminophenyl)ethyl)(methyl)carbamate

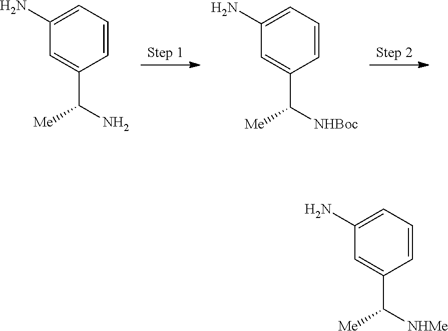 [SMILES: C[C@@H](N)c1cccc(N)c1 ; C[C@@H](NC(=O)OC(C)(C)C)c1cccc(N)c1 ; CN[C@H](C)c1cccc(N)c1]

### Step 1. Synthesis of tert-butyl (R)-(1-(3-aminophenyl)ethyl)carbamate

Na₂CO₃ (6.23 g, 58.7 mmol) was added to a solution of (R)-3-(1-aminoethyl) aniline (4 g, 29.4 mmol) and di-tert-butyl dicarbonate (6.41 g, 29.4 mmol) in THF (30 mL) and H₂O (10 mL) and the mixture stirred at 25° C. for 1 h. The reaction mixture was concentrated under reduced pressure and the residue diluted with H₂O (20 mL) and extracted with EtOAc (3×20 mL). The combined organics were evaporated to dryness under reduced pressure and the residue purified by MPLC (SiO₂, 1-5% EtOAc/PE) to give the title compound as a yellow oil (5.05 g, 73%).

### Step 2. Synthesis of (R)-3-(1-(methylamino)ethyl) aniline

To a solution of tert-butyl (R)-(1-(3-aminophenyl)ethyl)carbamate (5.05 g, 21.4 mmol) in THF (100 mL) was added LiAlH₄ (4.06 g, 107 mmol) at 0° C. and the mixture stirred at 50° C. for 3 h under N₂. The reaction mixture was quenched by addition Na₂SO₄. 10H₂O (5 g) at 0° C., filtered and the filtrate was concentrated under reduced pressure to give the title compound as a yellow oil (3.4 g, crude). LCMS m/z=151 [M+H]⁺.

### Intermediate A3. tert-butyl (R)-(1-(3-aminophenyl)ethyl)(methyl)carbamate

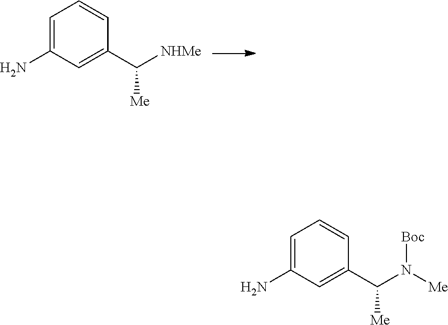 [SMILES: CN[C@H](C)c1cccc(N)c1 ; C[C@H](c1cccc(N)c1)N(C)C(=O)OC(C)(C)C]

To a solution of (R)-3-(1-(methylamino)ethyl) aniline (3.40 g, 22.6 mmol) in THF (30 mL) was added (Boc)₂₀ (2.96 g, 13.6 mmol) and the mixture stirred at 25° C. for 1 h. The reaction mixture was concentrated under reduced pressure and the residue purified by MPLC (SiO₂, 1-5% EtO Ac/PE) to give the title compound as a yellow solid (3.5 g, 62%).

### Intermediate A4 and A5. tert-butyl(R)-2-(5-aminopyridin-3-yl) piperidine-1-carboxylate and tert-butyl(S)-2-(5-aminopyridin-3-yl) piperidine-1-carboxylate

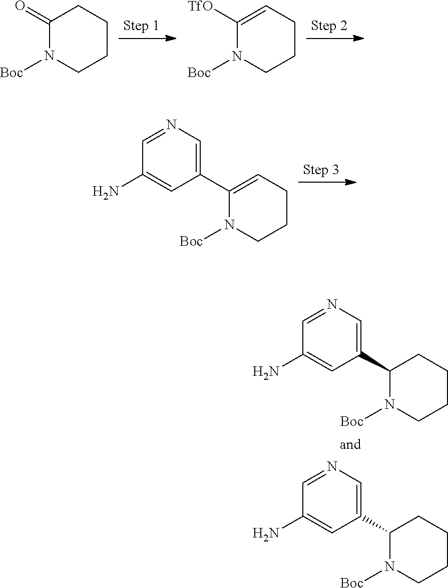 [SMILES: CC(C)(C)OC(=O)N1CCCCC1=O ; CC(C)(C)OC(=O)N1CCCC=C1OS(=O)(=O)C(F)(F)F ; CC(C)(C)OC(=O)N1CCCC=C1c1cncc(N)c1 ; CC(C)(C)OC(=O)N1CCCC[C@@H]1c1cncc(N)c1 ; CC(C)(C)OC(=O)N1CCCC[C@H]1c1cncc(N)c1]

### Step 1. Synthesis of tert-butyl 6-(((trifluoromethyl) sulfonyl)oxy)-3, 4-dihydropyridine-1 (2H)-carboxylate

KHMDS (1 M, 502 mL) in THF (250 mL) was added to a solution of tert-butyl 2-oxopiperidine-1-carboxylate (100 g, 502 mmol) in THF (500 mL) at −78° C. and the mixture stirred at −78° C. for 1 h before 1,1,1-trifluoro-N-phenyl-N-(trifluoromethylsulfonyl) methanesulfonamide (197 g, 552 mmol) was added to the mixture and the resulting mixture stirred at −78° C. for 1 h. The reaction mixture was slowly poured into H₂O (1000 mL) at 0-10° C. under nitrogen and extracted with EtOAc (3×1000 mL). The combined organics were washed with brine (2×1000 mL), dried (Na₂SO₄) and concentrated to afford the title compound as a yellow oil (220 g, crude) which was used without additional purification. ¹H NMR (400 MHz, CDCl₃): 5.14 (t, 1H), 3.47-3.42 (m, 2H), 2.11 (dt, 2H), 1.89 (s, 1H), 1.34 (s, 9H), 1.13-1.09 (m, 1H).

### Step 2. Synthesis of tert-butyl 5′-amino-5,6-dihydro-[2,3′-bipyridine]-1 (4H)-carboxylate

To a mixture of tert-butyl 6-(((trifluoromethyl) sulfonyl)oxy)-3,4-dihydropyridine-1 (2H)-carboxylate (63.41 g, 191 mmol) and (5-amino-3-pyridyl) boronic acid (33 g, 239 mmol) in dioxane (300 mL) and H₂O (100 mL) was added K₂CO₃ (126.64 g, 916 mmol) and Pd(dppf) C₁₂·CH₂Cl₂ (5.86 g, 7.18 mmol) at 25° C. and the mixture stirred at 100° C. for 1 h under N₂. The reaction mixture was partitioned between EtOAc (1000 mL) and H₂O (1000 mL) and the aqueous phase extracted with EtOAc (3×1000 mL). The combined organics were dried (Na₂SO₄) and evaporated to dryness in vacuo. The residue was purified by column chromatography (SiO₂, 33-100% EtOAc/PE) to give the title compound as a yellow solid (60 g, 91%). LCMS m/z=276 [M+H]⁺.

### Step 3. Synthesis of tert-butyl(R)-2-(5-aminopyridin-3-yl) piperidine-1-carboxylate and tert-butyl(S)-2-(5-aminopyridin-3-yl) piperidine-1-carboxylate

To a mixture of tert-butyl 5′-amino-5,6-dihydro-[2,3′-bipyridine]-1 (4H)-carboxylate (15 g, 54.5 mmol) in EtOAc (150 mL) was added Pd/C (15 g, 10% purity) at 25° C. and the mixture reaction stirred at 50° C. for 2 h under H₂. The reaction mixture was filtered and the filtrate was concentrated under reduced pressure to give tert-butyl 2-(5-amino-3-pyridyl) piperidine-1-carboxylate as a white solid (15 g, crude).

The racemate was separated by SFC (Chiralcel C₂-3, 100×4.6 mm, 3 μm; EtOH (0.1% IPAm) in CO₂ to afford:

Peak 1. Intermediate A4, tert-butyl(R)-2-(5-aminopyridin-3-yl) piperidine-1-carboxylate (23 g, 60%).

Peak 2. Intermediate A5, tert-butyl(S)-2-(5-aminopyridin-3-yl) piperidine-1-carboxylate (23 g, 60%).

### Intermediate A6. tert-butyl ((5-aminopyridin-3-yl)methyl)(methyl)carbamate

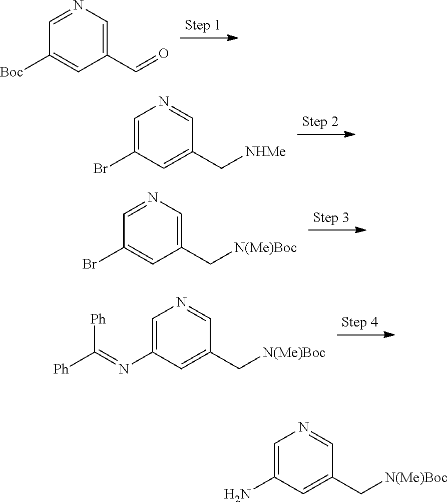

### Step 1. Synthesis of 1-(5-bromopyridin-3-yl)-N-methylmethanamine

A mixture of 5-bromopyridine-3-carbaldehyde (5 g, 26.88 mmol) in methanamine (27.83 g, 269 mmol, 30% purity) was stirred at 25° C. for 12 h under N₂ and NaBH₄ (2.03 g, 53.8 mmol) added at 0° C. The resulting mixture was stirred at 25° C. for 2 h under N₂. The reaction mixture was quenched by addition saturated 0.5 M HCl (10 mL) at 0° C. and extracted with EtOAc (3×10 mL). The combined organics were dried (Na₂SO₄) and concentrated under reduced pressure to give the title compound as a yellow oil (5.30 g). LCMS m/z=201 [M+H]⁺.

### Step 2. Synthesis of tert-butyl ((5-bromopyridin-3-yl)methyl)(methyl)carbamate

To a solution of 1-(5-bromopyridin-3-yl)-N-methylmethanamine (5.20 g, 25.9 mmol) and (Boc)₂O (5.64 g, 25.9 mmol) in THF (52 mL) and H₂O (13 mL) was added Na₂CO₃ (5.48 g, 51.7 mmol) and the mixture stirred at 25° C. for 2 h. The reaction was diluted with H₂O (80 mL) and extracted with EtOAc (3×50 mL). The combined organics were dried (Na₂SO₄) and concentrated under reduced pressure. The residue was purified by MPLC (SiO₂, 50% EtOAc/PE) to give the title compound as a yellow oil (4.50 g, 57.8%). LCMS m/z=301 [M+H]⁺.

### Step 3. Synthesis of tert-butyl ((5-((diphenylmethylene)amino)pyridin-3-yl)methyl)(methyl)carbamate

To a solution of tert-butyl ((5-bromopyridin-3-yl)methyl)(methyl)carbamate (4.40 g, 14.61 mmol) and diphenylmethanimine (2.91 g, 16.07 mmol) in dioxane (70 mL) was added Pd₂ (dba)₃ (1.34 g, 1.46 mmol), Xantphos (1.69 g, 2.92 mmol) and Cs₂CO₃ (14.28 g, 43.83 mmol) and the mixture stirred at 100° C. for 3 h under N₂. The reaction mixture was filtered and the filtrate concentrated under reduced pressure and the residue purified by MPLC (SiO₂, 50% EtOAc/PE) to give the title compound as a yellow oil (3.50 g, 60%). LCMS m/z=402 [M+H]⁺.

### Step 4. Synthesis of tert-butyl ((5-aminopyridin-3-yl)methyl)(methyl)carbamate

To a solution of tert-butyl ((5-((diphenylmethylene)amino)pyridin-3-yl)methyl)(methyl)carbamate (3.40 g, 8.47 mmol) in MeOH (40 mL) was added NH₂OH·HCl (1.18 g, 16.94 mmol) and NaOAc (1.39 g, 16.94 mmol) and the mixture stirred at 25° C. for 1 h. The reaction mixture was concentrated under reduced pressure and the residue extracted with EtOAc (3×50 mL). The combined organics were dried (Na₂SO₄) and concentrated under reduced pressure to give a residue that was purified by MPLC (SiO₂, EtOAc) to give the title compound as a yellow oil (2.00 g, 99.5%). LCMS m/z=238 [M+H]⁺.

### Intermediate A7. tert-butyl (R)-(1-(5-aminopyridin-3-yl)butyl)carbamate

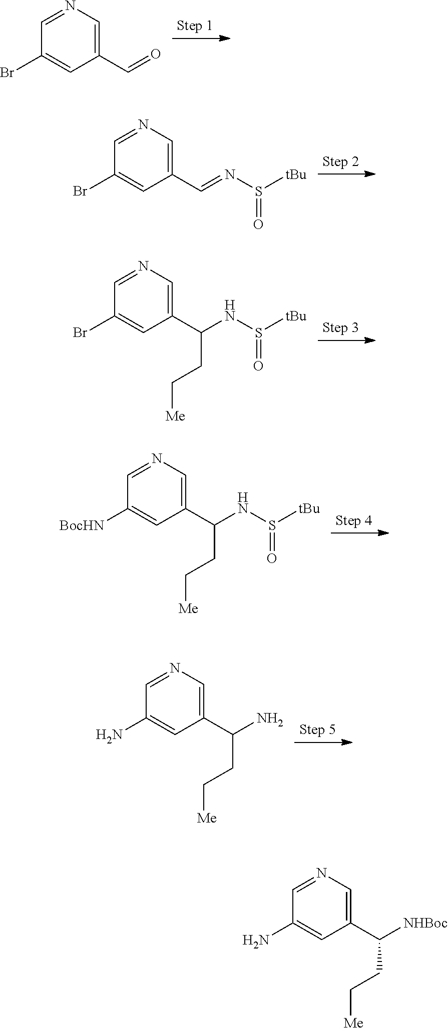

### Step 1. Synthesis of (E)-N-((5-bromopyridin-3-yl)methylene)-2-methylpropane-2-sulfinamide

To a solution of 5-bromonicotinaldehyde (5 g, 26.88 mmol) and 2-methylpropane-2-sulfinamide (3.58 g, 29.57 mmol) in THF (60 mL) was added Ti(OEt)₄ (7.97 g, 34.95 mmol) and the mixture stirred at 40° C. for 12 h. The reaction mixture was added to H₂O (50 mL) and then the solids removed by filtration and the filter cake washed with EtOAc (3×20 mL). The aqueous phase was extracted with additional EtOAc (3×50 mL) and the combined organics were dried (Na₂SO₄) and evaporated to dryness. The residue was purified by MPLC (SiO₂, 1-50% EtO Ac/PE) to give the title compound as a white solid (6 g, 77%). LCMS m/z=291 [M+H]⁺.

### Step 2. Synthesis of N-(1-(5-bromopyridin-3-yl)butyl)-2-methylpropane-2-sulfinamide

To a solution of (E)-N-((5-bromopyridin-3-yl)methylene)-2-methylpropane-2-sulfinamide (Step 1, 3.90 g, 13.49 mmol) in THF (20 mL) was added propylmagnesium chloride (2 M, 10.11 mL) at −78° C. and the mixture stirred at 0° C. for 12 h. The reaction mixture was filtered and the filtrate was concentrated under reduced pressure and the residue purified by column chromatography (SiO₂, 0-50% EtOAc) to give the title compound as a yellow solid (5.10 g, crude). LCMS m/z=335 [M+H]⁺.

### Step 3. Synthesis of tert-butyl(5-(1-((tert-butylsulfinyl)amino)butyl)pyridin-3-yl)carbamate

To a solution of tert-butyl carbamate (3.51 g, 30 mmol) and N-(1-(5-bromopyridin-3-yl)butyl)-2-methylpropane-2-sulfinamide (5.00 g, 15 mmol) in dioxane (30 mL) was added Cs₂CO₃ (7.33 g, 22.5 mmol) and Pd₂ (dba)₃ (1.37 g, 1.50 mmol), XPhos (715 mg, 1.50 mmol) and the mixture stirred at 100° C. for 2 h under N₂. The reaction mixture was filtered and the filtrate concentrated under reduced pressure and the residue purified by column chromatography (SiO₂, 0-50% EtOAc/PE) to give the title compound as a white solid (3.00 g, 54%). LCMS m/z=370 [M+H]⁺.

### Step 4. Synthesis of 5-(1-aminobutyl)pyridin-3-amine

A solution of tert-butyl(5-(1-((tert-butylsulfinyl)amino)butyl)pyridin-3-yl)carbamate (3.00 g, 8.12 mmol) HCl/EtOAc (1 mL) was stirred at 25° C. for 2 h. The reaction mixture was concentrated under N₂ to give the title compound as a yellow solid (2.60 g, crude). LCMS m/z=166 [M+H]⁺.

### Step 5. Synthesis of tert-butyl (R)-(1-(5-aminopyridin-3-yl)butyl)carbamate

To a solution of 5-(1-aminobutyl)pyridin-3-amine (1.10 g, 6.66 mmol) in dioxane (10 mL) and H₂O (5 mL) was added NaHCO₃ (559 mg, 6.66 mmol) and (Boc)₂₀ (581 mg, 2.66 mmol) and the mixture stirred at 25° C. for 12 h. The reaction mixture was filtered and the filtrate concentrated under reduced pressure. The residue was purified by column chromatography (SiO₂, 0-50% EtOAc/PE) to give tert-butyl(1-(5-aminopyridin-3-yl)butyl)carbamate which was further separated by SFC (DAICEL CHIRALPAK AD, 250×30 mm, 10 μm); 30% EtOH (0.1% NH₄OH) in CO₂ to afford:

Peak 2. Intermediate A7. tert-butyl (R)-(1-(5-aminopyridin-3-yl)butyl)carbamate (white solid, 210 mg, 11.9%). LCMS m/z=266 [M+H]⁺.

### Intermediate A8. tert-butyl(S)-(1-(5-aminopyridin-3-yl) propyl)carbamate or tert-butyl (R)-(1-(5-aminopyridin-3-yl) propyl)carbamate

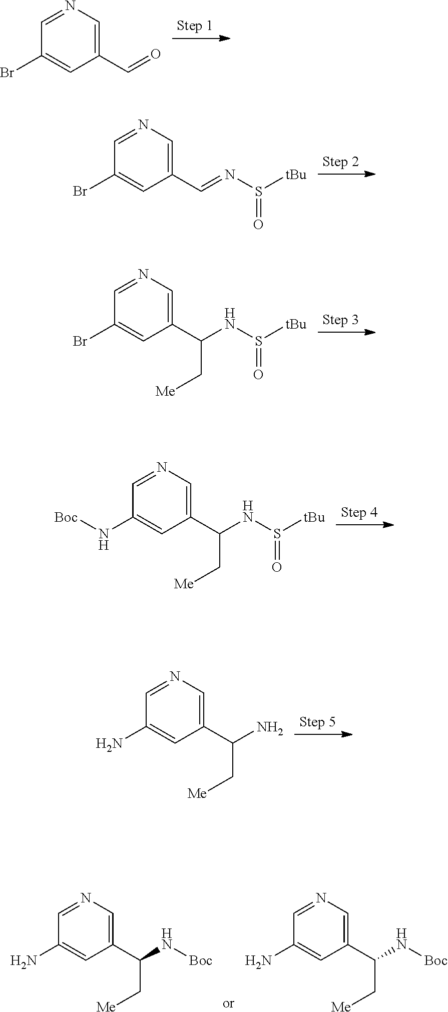

The title compounds were prepared using an analogous 5-Step method to that described for Intermediate A7. Chiral-SFC (DAICEL CHIRALPAK AD, 250×30 mm, 10 μm); 33% EtOH (+0.1% NH₄OH in CO₂)) afforded:

Peak 2, Intermediate A8. tert-butyl(S)-(1-(5-aminopyridin-3-yl) propyl)carbamate or tert-butyl (R)-(1-(5-aminopyridin-3-yl) propyl)carbamate (yellow solid: 1.1 g, 15%); LCMS m/z 252 [M+H]⁺.

### Intermediate A9. tert-butyl(S)-((5-aminopyridin-3-yl)(cyclopropyl)methyl)carbamate or tert-butyl(R)-((5-aminopyridin-3-yl)(cyclopropyl)methyl)carbamate

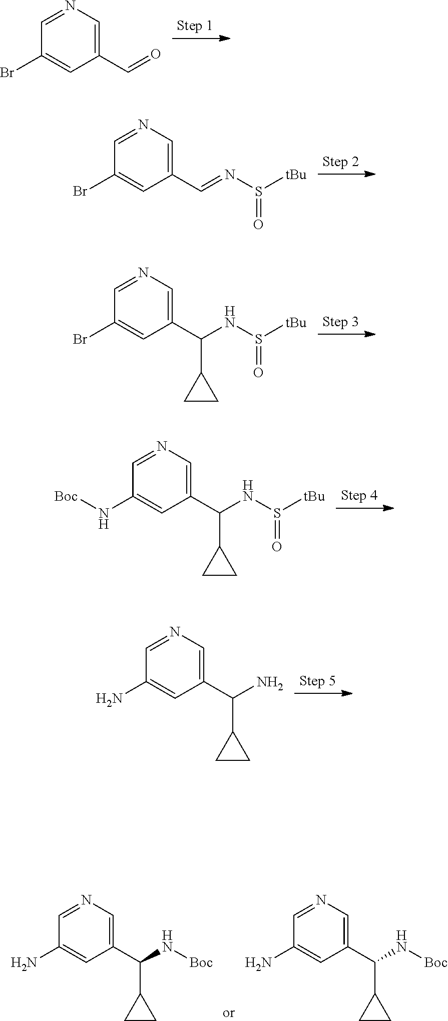

The title compound was prepared using an analogous 5-Step method to that described for Intermediate A7.

Chiral-SFC (DAICEL CHIRALPAK AD, 250×50 mm, 10 μm); 30% EtOH (+0.1% NH₄OH in CO₂)) afforded:

Peak 1, Intermediate A9. tert-butyl(S)-((5-aminopyridin-3-yl)(cyclopropyl)methyl)carbamate or tert-butyl(R)-((5-aminopyridin-3-yl)(cyclopropyl)methyl)carbamate (yellow oil: 630 mg, 13%); LCMS m/z=252 [M+H]⁺.

### Intermediate A10 and A11. tert-butyl (R)-(1-(5-aminopyridin-3-yl)-2-ethoxyethyl)carbamate and tert-butyl(S)-(1-(5-aminopyridin-3-yl)-2-ethoxyethyl)carbamate

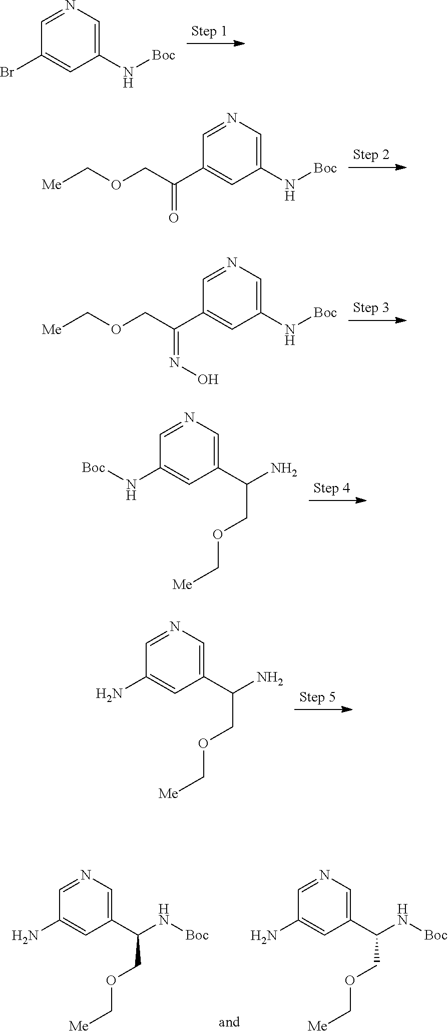

### Step 1. Synthesis of tert-butyl(5-(2-ethoxyacetyl)pyridin-3-yl)carbamate

n-BuLi (2.5 M in hexanes, 14.65 mL) was added dropwise to a solution of tert-butyl N-(5-bromo-3-pyridyl)carbamate (5.00 g, 18.3 mmol) in THF (150 mL) at −60° C. The mixture was stirred at −60° C. for 30 min under N₂ and ethyl 2-ethoxyacetate (2.90 g, 22 mmol) added and the mixture stirred at 25° C. for 2 h under N₂. The reaction mixture was quenched by addition of sat aq NH₄Cl (20 mL) at 0° C. and then diluted with H₂O (20 mL) and extracted with EtOAc (3×20 mL). The combined organics were dried (Na₂SO₄) and concentrated under reduced pressure. The residue was purified by prep-HPLC (Waters Xbridge Prep OBD C18, 150×40 mm, 10 μm; 15-45% MeCN/H₂O(NH₄OH+NH₄HCO₃)) to give the title compound as a yellow oil (1.40 g, 27%). LCMS m/z=281 [M+H]⁺.

### Step 2. Synthesis of tert-butyl(E)-(5-(2-ethoxy-1-(hydroxyimino)ethyl)pyridin-3-yl)carbamate

To mixture of tert-butyl(5-(2-ethoxyacetyl)pyridin-3-yl)carbamate (1.28 g, 4.57 mmol) in EtOH (20 mL) was added NH₂OH·HCl (381 mg, 5.48 mmol) and NaOAc (749 mg, 9.13 mmol) and the mixture stirred at 75° C. for 2 h. The reaction mixture was quenched by addition sat aq NH₄Cl (20 mL) at 0° C., diluted with H₂O (20 mL) and extracted with EtOAc (3×20 mL). The combined organics were dried (Na₂SO₄) and concentrated under reduced pressure to give the title compound (1.30 g, 96%). LCMS m/z=291 [M+H]⁺.

### Step 3. Synthesis of tert-butyl(5-(1-amino-2-ethoxyethyl)pyridin-3-yl)carbamate

A mixture of tert-butyl(E)-(5-(2-ethoxy-1-(hydroxyimino)ethyl)pyridin-3-yl)carbamate (1.05 g, 3.56 mmol), Pd/C (600 mg, 3.56 mmol, 10% purity), NH₄OH (1 mL) and IPA (10 mL) was degassed and purged with H₂ (50 psi) for 3 h and then the mixture stirred at 50° C. for 16 h under H₂ (50 psi). The reaction mixture was filtered and the filtrate was concentrated under reduced pressure to give the title compound as a white solid (960 mg, 96%). LCMS m/z=282 [M+H]⁺.

### Step 4. Synthesis of 5-(1-amino-2-ethoxyethyl)pyridin-3-amine hydrochloride

A mixture of tert-butyl(5-(1-amino-2-ethoxyethyl)pyridin-3-yl)carbamate (800 mg, 2.84 mmol) in EtOAc (6 mL) and HCl/EtOAc (3 mL) was stirred at 25° C. for 12 h. The reaction mixture was filtered and the filtrate was concentrated under reduced pressure to give the title compound as a white solid (400 mg, 77%). LCMS m/z=182 [M+H]⁺.

### Step 5. Synthesis of tert-butyl (R)-(1-(5-aminopyridin-3-yl)-2-ethoxyethyl)carbamate and tert-butyl(S)-(1-(5-aminopyridin-3-yl)-2-ethoxyethyl)carbamate

A mixture of 5-(1-amino-2-ethoxyethyl)pyridin-3-amine hydrochloride (400 mg, 1.84 mmol), Boc₂O (160 mg, 0.735 mmol), NaHCO₃ (309 mg, 3.67 mmol) in THF (15 mL) and H₂O (5 mL) was stirred at 25° C. for 1 h. The reaction mixture was concentrated under reduced pressure and the residue diluted with H₂O (10 mL) and extracted with EtOAc (3×10 mL).

The combined organics were dried (Na₂SO₄) and concentrated under reduced pressure to give tert-butyl(1-(5-aminopyridin-3-yl)-2-ethoxyethyl)carbamate which was separated by chiral SFC (DAICEL CHIRALPAK IC, 250×30 mm, 10 μm); 28% EtOH (0.1% NH₄OH) in CO₂) to afford:

Peak 1, Intermediate A10. tert-butyl (R)-(1-(5-aminopyridin-3-yl)-2-ethoxyethyl)carbamate or tert-butyl(S)-(1-(5-aminopyridin-3-yl)-2-ethoxyethyl)carbamate.

Peak 2, Intermediate A11, tert-butyl (R)-(1-(5-aminopyridin-3-yl)-2-ethoxyethyl)carbamate or tert-butyl(S)-(1-(5-aminopyridin-3-yl)-2-ethoxyethyl)carbamate. LCMS m/z=282 [M+H]⁺.

### Intermediate A12. 5-((ethylamino)methyl)pyridin-3-amine

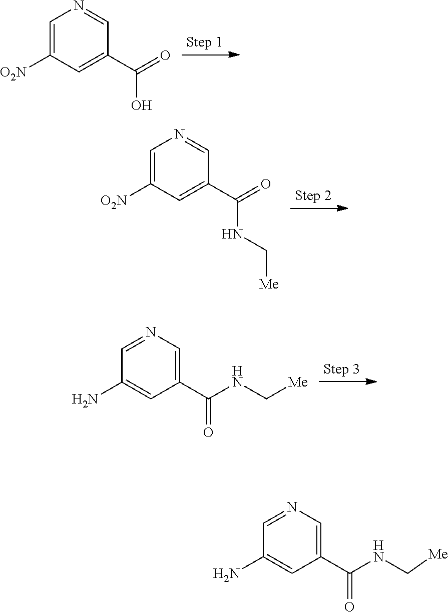 [SMILES: O=C(O)c1cncc([N+](=O)[O-])c1 ; CCNC(=O)c1cncc([N+](=O)[O-])c1 ; CCNC(=O)c1cncc(N)c1 ; CCNC(=O)c1cncc(N)c1]

### Step 1. Synthesis of N-ethyl-5-nitronicotinamide

To a solution of 5-nitronicotinic acid (5 g, 29.7 mmol) in pyridine (10 mL) was added ethanamine; hydrochloride (12.13 g, 148.7 mmol) and EDCI (8.55 g, 44.61 mmol) and the mixture stirred at 25° C. for 1 h. The reaction mixture was concentrated under reduced pressure and the residue purified by MPLC (SiO₂, 0-100% EtOAc/PE) to give the title compound as a white solid (3.5 g, 60%). LCMS m/z=196 [M+H]⁺.

### Step 2. Synthesis of 5-amino-N-ethylnicotinamide

To a mixture of N-ethyl-5-nitronicotinamide (3.4 g, 17.42 mmol) in MeOH (5 mL) was slowly added Pd/C (4 g, 25.62 mmol, 10% purity) and the mixture was stirred at 25° C. for 12 h under H₂ (15 psi). The reaction mixture was filtered and the filtrate was concentrated under reduced pressure to give the title compound as a yellow solid (2.8 g, 97%). LCMS m/z=166 [M+H]⁺.

### Step 3. Synthesis of 5-((ethylamino)methyl)pyridin-3-amine

To a solution of N-ethyl-5-nitronicotinamide (1.5 g, 9.08 mmol) was added BH₃. THF (1M, 45.4 mL) at 0° C. under N₂ and the mixture stirred at 50° C. for 2 h under N₂. The reaction mixture was quenched with 0.1 M HCl (50 mL) at 0° C., filtered and concentrated under reduced pressure. The residue was purified by prep-HPLC (Xbridge Prep OBD C18, 150×40 mm, 10 μm; 1-5% MeCN/H₂O(NH₄OH+NH₄HCO₃)) to give the title compound as a yellow oil (550 mg, 40%). LCMS m/z=152 [M+H]⁺.

### Intermediate A13. tert-butyl(S)-2-(5-aminopyridin-3-yl) pyrrolidine-1-carboxylate

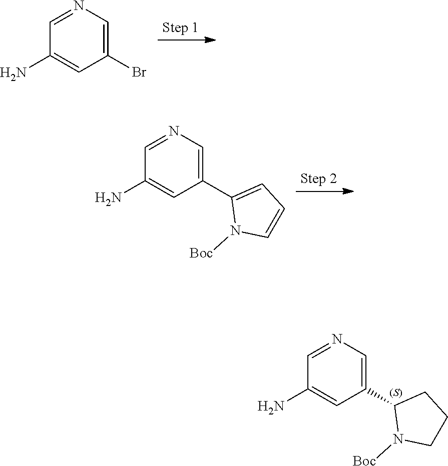

### Step 1. Synthesis of tert-butyl 2-(5-aminopyridin-3-yl)-1H-pyrrole-1-carboxylate

A mixture of 5-bromopyridin-3-amine (1.50 g, 8.67 mmol), tert-butyl 2-(4,4,5,5-tetramethyl-1,3,2-dioxaborolan-2-yl) pyrrole-1-carboxylate (2.80 g, 9.54 mmol), Pd(dppf) C₁₂ (634 mg, 0.87 mmol) and K₃PO₄ (3.68 g, 17.3 mmol) in H₂O (15 mL) and THF (40 mL) was stirred at 80° C. for 1 h under N₂. The reaction mixture was filtered and the filtrate concentrated under reduced pressure and the residue purified by column chromatography (SiO₂, 0-50% EtOAc/PE) to give the title compound as a yellow solid (2 g, 89%). LCMS m/z=260 [M+H]⁺.

### Step 2. Synthesis of tert-butyl(S)-2-(5-aminopyridin-3-yl) pyrrolidine-1-carboxylate

To a solution of tert-butyl 2-(5-aminopyridin-3-yl)-1H-pyrrole-1-carboxylate (Step 1, 1 g, 3.86 mmol) in EtOH (20 mL) was added Pd/C (1.00 g, 10% purity) at 25° C. The mixture was stirred at 60° C. for 25 h under H₂ (15 psi). The reaction mixture was filtered and the filtrate concentrated under reduced pressure to give a residue to give tert-butyl 2-(5-aminopyridin-3-yl) pyrrolidine-1-carboxylate as a yellow solid (700 mg, 69%). The solid was further purified by prep-SFC (DAICEL CHIRALPAK IG, 250×30 mm, 10 μm); 33% IPA (0.1% NH₄OH) in CO₂) to afford:

Peak 2 Intermediate A13: tert-butyl(S)-2-(5-aminopyridin-3-yl) pyrrolidine-1-carboxylate (yellow solid, 330 mg, 47%). LCMS m/z=264 [M+H]⁺.

### Intermediate A14 and A15. (S)-5-(1-(methylamino)ethyl)pyridin-3-amine and (R)-5-(1-(methylamino)ethyl)pyridin-3-amine

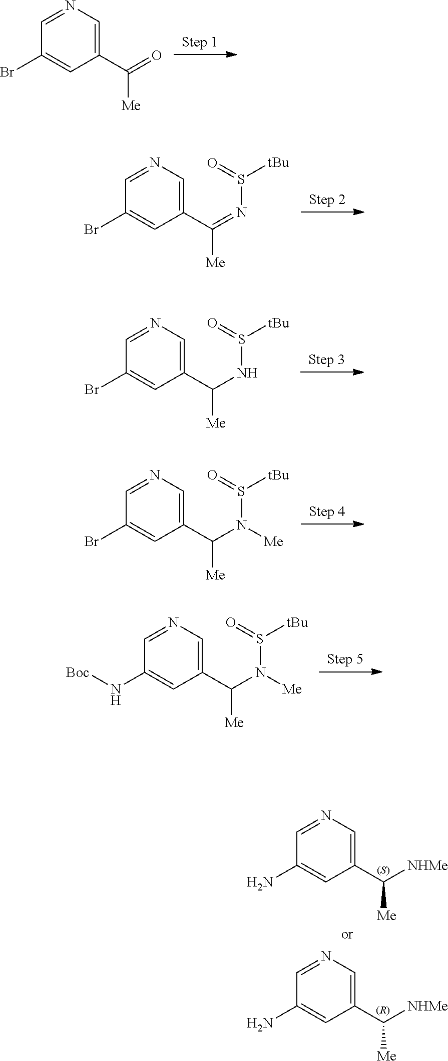

### Step 1. Synthesis of (Z)—N-(1-(5-bromopyridin-3-yl)ethylidene)-2-methylpropane-2-sulfinamide

A mixture of 1-(5-bromo-3-pyridyl) ethanone (25 g, 125 mmol) in THF (300 mL) was added 2-methylpropane-2-sulfinamide (15.91 g, 131 mmol) and Ti(OEt)₄ (37.06 g, 162 mmol) and the mixture stirred at 45° C. for 12 h. The reaction mixture was diluted with H₂O (300 mL), filtered and extracted with EtOAc (4×200 mL). The combined organics were concentrated under reduced pressure and the residue was purified by column chromatography (SiO₂, 0-25% EtO Ac/PE) to give the title compound as a yellow oil (33 g, 87%). LCMS m/z=303 [M+H]⁺.

### Step 2. Synthesis of N-(1-(5-bromopyridin-3-yl)ethyl)-2-methylpropane-2-sulfinamide

NaBH₄ (5.75 g, 152 mmol) was added to a solution of (Z)—N-(1-(5-bromopyridin-3-yl)ethylidene)-2-methylpropane-2-sulfinamide (Step 1, 30 g, 99 mmol) in MeOH (200 mL) at 0° C. and the mixture stirred at 20° C. for 1 h. The reaction mixture was quenched by addition of NH₄Cl aq. (200 ml) at 0° C. and Na₂CO₃ aq. was added until pH=10. The mixture was extracted with EtOAc (4×100 mL). The combined organics were dried (Na₂SO₄) and concentrated under reduced pressure. The reaction was purified by column chromatography (SiO₂, 0-100% EtOAc/PE) to give the title compound as a white solid (22 g, 73%). LCMS m/z=307 [M+H]⁺.

### Step 3. Synthesis of N-(1-(5-bromopyridin-3-yl)ethyl)-N, 2-dimethylpropane-2-sulfinamide

NaH (1.18 g, 29.5 mmol, 60% purity) was added to a solution of iodomethane (4.65 g, 32.8 mmol) in DMF (40 mL) at −5° C. under N₂ and the mixture stirred at −5° C. for 20 min. To this was added N-(1-(5-bromopyridin-3-yl)ethyl)-2-methylpropane-2-sulfinamide (Step 2, 5 g, 16.4 mmol) and the mixture was stirred at −5° C. for 20 min. The reaction was quenched by the addition of NH₄Cl (100 mL) at 0° C. and the mixture extracted with EtOAc (4×200 mL). The combined organics were evaporated to dryness and the residue purified by prep-HPLC (Phenomenex C18 250×70 mm, 10 μm; 30-55% MeCN/H₂O(NH₄OH+NH₄HCO₃)) to give the title compound as a brown oil (6.3 g, crude).

### Step 4. Synthesis of tert-butyl(5-(1-((tert-butylsulfinyl)(methyl)amino)ethyl)pyridin-3-yl)carbamate

A mixture N-(1-(5-bromopyridin-3-yl)ethyl)-N, 2-dimethylpropane-2-sulfinamide (Step 3, 5.2 g, 16.3 mmol), tert-butyl carbamate (3.82 g, 32.6 mmol), Cs₂CO₃ (7.96 g, 24.4 mmol), XPhos (776 mg, 1.63 mmol) and Pd₂ (dba)₃ (1.49 g, 1.63 mmol) in dioxane (40 mL) was degassed and purged with N₂ (×3) and the mixture was stirred at 100° C. for 2 h under N₂. The reaction mixture was filtered and the filtrate was concentrated under reduced pressure and the residue purified by column chromatography (SiO₂, 0-50% EtOAc/PE) to give the title compound as a yellow oil (4.5 g, 77%). LCMS m/z=356 [M+H]⁺.

### Step 5. Synthesis of (S)-5-(1-(methylamino)ethyl)pyridin-3-amine and (R)-5-(1-(methylamino)ethyl)pyridin-3-amine

HCl/EtOAc (20 mL) was added to a solution of tert-butyl(5-(1-((tert-butylsulfinyl)(methyl)amino)ethyl)pyridin-3-yl)carbamate (Step 5, 3 g, 8.44 mmol) in EtOAc (10 mL) and the mixture stirred at 25° C. for 1 h. The reaction mixture was filtered and concentrated under reduced pressure to give a 5-(1-(methylamino)ethyl)pyridin-3-amine as a white solid (600 mg, 25%) which was separated by prep-SFC (DAICEL CHIRALPAK IG, 250×30 mm, 10 μm); 30% Heptane/EtOH (0.1% NH₄OH) in CO₂ to afford:

Peak 1 Intermediate A14 (S)-5-(1-(methylamino)ethyl)pyridin-3-amine or (R)-5-(1-(methylamino)ethyl)pyridin-3-amine (brown oil, 240 mg, 18%); LCMS m/z=152 [M+H]⁺.

Peak 2 Intermediate A15 (S)-5-(1-(methylamino)ethyl)pyridin-3-amine or (R)-5-(1-(methylamino)ethyl)pyridin-3-amine (brown oil, 240 mg, 18%); LCMS m/z=152 [M+H]⁺.

### Intermediate A16 and A17. tert-butyl(S)-(1-(5-aminopyridin-3-yl)ethyl)carbamate and tert-butyl (R)-(1-(5-aminopyridin-3-yl)ethyl)carbamate

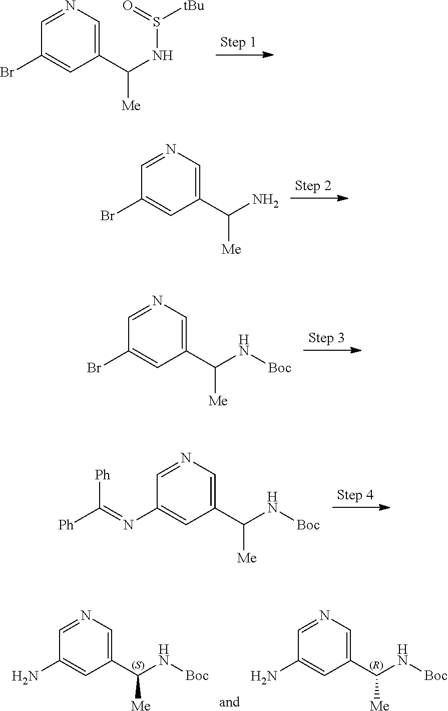

### Step 1. Synthesis of 1-(5-bromopyridin-3-yl) ethan-1-amine

A mixture of N-(1-(5-bromopyridin-3-yl)ethyl)-2-methylpropane-2-sulfinamide (Intermediate A14 Step 2, 14 g, 45.87 mmol) in EtOAc (1 mL) and HCl/EtOAc (2 mL) was stirred at 25° C. for 1 h. The reaction mixture was concentrated under N₂ to give the title compound as a yellow oil (9 g, 97%). LCMS m/z=201 [M+H]⁺.

### Step 2. Synthesis of tert-butyl(1-(5-bromopyridin-3-yl)ethyl)carbamate

To mixture of 1-(5-bromopyridin-3-yl) ethan-1-amine (Step 1, 8.5 g, 42.3 mmol) in dioxane (80 mL) and H₂O (15 mL) was added Boc₂O (10.15 g, 46.50 mmol) and NaHCO₃ (7.10 g, 84.6 mmol) and the mixture stirred at 25° C. for 1 h. The solids were removed by filtration and the filtrate extracted with EtOAc (3×150 mL). The combined organics were dried (Na₂SO₄) and evaporated to dryness in vacuo. The residue was purified by column chromatography on silica gel (0-50% EtOAc/PE) to give the title compound as a yellow oil (8.6 g, 67%). LCMS m/z=301 [M+H]⁺.

### Step 3. Synthesis of tert-butyl(1-(5-((diphenylmethylene)amino)pyridin-3-yl)ethyl)carbamate

To a mixture of tert-butyl(1-(5-bromopyridin-3-yl)ethyl)carbamate (Step 2, 8.5 g, 28.22 mmol) and diphenylmethanimine (5.63 g, 31.04 mmol) in dioxane (2 mL) was added Pd₂ (dba)₃ (1.29 g, 1.41 mmol) and Xantphos (1.63 g, 2.82 mmol) and Cs₂CO₃ (18.39 g, 56.44 mmol) and the mixture stirred at 100° C. for 1 h under N₂. The reaction mixture was concentrated under reduced pressure and the residue was purified by column chromatography on silica gel (0-50% EtO Ac/PE) to give the title compound as a yellow oil (11 g, 97%). LCMS m/z=402 [M+H]⁺.

### Step 4. Synthesis of of tert-butyl(S)-(1-(5-aminopyridin-3-yl)ethyl)carbamate and tert-butyl (R)-(1-(5-aminopyridin-3-yl)ethyl)carbamate

To the mixture of tert-butyl(1-(5-((diphenylmethylene)amino)pyridin-3-yl)ethyl)carbamate (Step 3, 10 g, 24.9 mmol) in MeOH (100 mL) was added NH₂OH·HCl (2.60 g, 37.4 mmol) and NaOAc (3.06 g, 37.4 mmol) and the mixture stirred at 25° C. for 1 h. The reaction mixture was filtered and the filtrate concentrated under reduced pressure and the residue purified by column chromatography on silica gel (0-50% MeOH/DCM) followed by chiral-SFC (DAICEL CHIRALPAK IC, 250×30 mm, 10 μm); 40% EtOH (0.1% NH₄OH) in CO₂ to afford:

Peak 1, Intermediate A16. tert-butyl(S)-(1-(5-aminopyridin-3-yl)ethyl)carbamate or tert-butyl (R)-(1-(5-aminopyridin-3-yl)ethyl)carbamate (yellow oil, 1.9 g, 39%); LCMS m/z=238 [M+H]⁺.

Peak 2, Intermediate A17. tert-butyl(S)-(1-(5-aminopyridin-3-yl)ethyl)carbamate or tert-butyl (R)-(1-(5-aminopyridin-3-yl)ethyl)carbamate (yellow oil, 1.8 g, 38%); LCMS m/z=238 [M+H]⁺.

### Intermediate A18. 5-(3-aminophenyl) piperazin-2-one

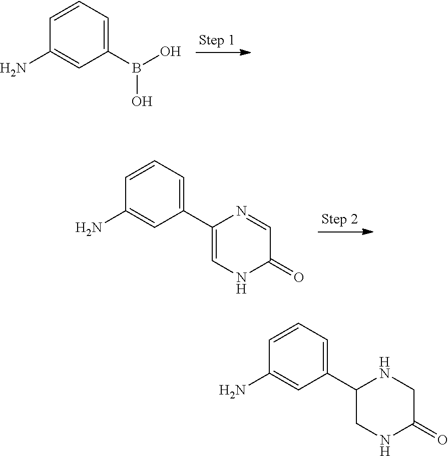

### Step 1. Synthesis of 5-(3-aminophenyl) pyrazin-2 (1H)-one

To a solution of (3-aminophenyl) boronic acid (1 g, 7.30 mmol, 1 eq) and 5-bromopyrazin-2 (1H)-one (1.28 g, 7.30 mmol) in H₂O (5 mL) and EtOH (20 mL) was added K₃PO₄ (3.10 g, 14.6 mmol) and cataCXiumA Pd G2 (488 mg, 0.73 mmol) and the mixture stirred at 80° C. for 1 h under N₂. The reaction mixture was filtered and the filtrate was concentrated under reduced pressure and the residue was purified by prep-HPLC (Xbridge BEH C18 250×50 mm, 10 μm; 1-35% MeCN/H₂O(NH₄OH+NH₄HCO₃)) to give the title compound as a yellow sold (1.18 g, 86%). LCMS m/z=188 [M+H]⁺.

### Step 2. Synthesis of 5-(3-aminophenyl) piperazin-2-one

To a solution of 5-(3-aminophenyl) pyrazin-2 (1H)-one (Step 1, 1 g, 5.34 mmol) in EtOAc (20 mL) was added Pd/C (1 g, 5.34 mmol, 10% purity) and the mixture stirred at 50° C. for 12 h under H₂. The reaction mixture was filtered and the filtrate was concentrated under reduced pressure to give the title compound as a white solid (268 mg, 26%). LCMS m/z=192 [M+H]⁺.

### Intermediate A19. tert-butyl(1-(5-aminopyridin-3-yl)-3-methoxypropyl)carbamate

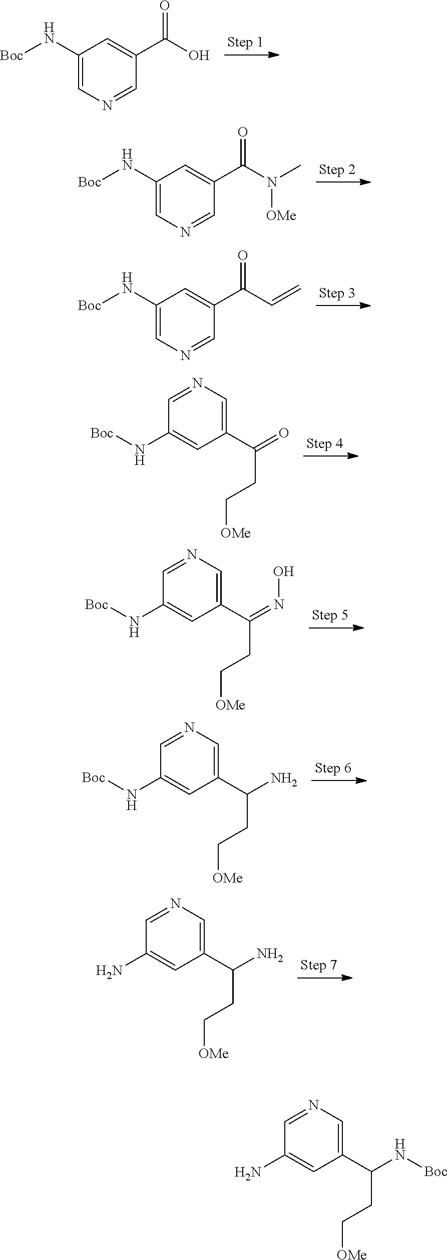

### Step 1. Synthesis of tert-butyl(5-(methoxy(methyl)carbamoyl)pyridin-3-yl)carbamate

DIPEA (2.17 g, 16.8 mmol) and HATU (4.79 g, 12.6 mmol) was added to the mixture of 5-((tert-butoxycarbonyl)amino) nicotinic acid (2 g, 8.39 mmol) and N-methoxymethanamine hydrochloride (1.23 g, 12.59 mmol) in DMSO (10 mL) and the mixture stirred at 25° C. for 2 h. The mixture was diluted with H₂O (50 mL) and extracted with EtOAc (4×100 mL). The combined organics were evaporated to dryness in vacuo and the residue purified by column chromatography on silica gel (0-50% EtOAc/PE) to give the title compound as a white oil (2 g, 84%). LCMS m/z=282 [M+H]⁺.

### Step 2. Synthesis of tert-butyl(5-acryloylpyridin-3-yl)carbamate

To a mixture of tert-butyl(5-(methoxy(methyl)carbamoyl)pyridin-3-yl)carbamate (Step 1, 500 mg, 1.78 mmol) in THF (6 mL) was added bromo (vinyl) magnesium (1M in THF, 6.22 mL) at 0° C. and the mixture stirred at 0° C. for another 1 min. The mixture was stirred at 25° C. for 1 h under N₂. The reaction mixture was quenched with saturated aqueous NH₄Cl solution (5 mL) at 0° C. and extracted with EtOAc (3×10 mL). The combined organics were dried (Na₂SO₄) and concentrated under reduced pressure to give the title compound as a yellow solid (700 mg, 79%).

### Step 3. Synthesis of tert-butyl(5-(3-methoxypropanoyl)pyridin-3-yl)carbamate

NaOMe (152 mg, 2.82 mmol) was added to a solution of tert-butyl(5-acryloylpyridin-3-yl)carbamate (Step 2, 350 mg, 1.41 mmol) in MeOH (6 mL) and the mixture stirred at 25° C. for 2 h. The reaction mixture was concentrated under reduced pressure and the residue purified by column chromatography on silica gel (0-100% EtOAc/PE) to give the title compound as a yellow oil (200 mg, 25%). LCMS m/z=281 [M+H]⁺.

### Step 4. Synthesis of tert-butyl(Z)-(5-(1-(hydroxyimino)-3-methoxypropyl)pyridin-3-yl)carbamate

TEA (361 mg, 3.57 mmol) and NH₂OH·HCl (186 mg, 2.68 mmol) were added to a solution of tert-butyl(5-(3-methoxypropanoyl)pyridin-3-yl)carbamate (Step 3, 250 mg, 0.89 mmol) in EtOH (5 mL) and the mixture stirred at 70° C. for 12 h. The reaction mixture was filtered and the filtrate concentrated under reduced pressure and the residue was purified by column chromatography on silica gel (0-100% EtOAc/PE) to give the title compound as a yellow oil (170 mg, 64%). LCMS m/z=296 [M+H]⁺.

### Step 5. Synthesis of tert-butyl(5-(1-amino-3-methoxypropyl)pyridin-3-yl)carbamate

To the mixture of tert-butyl(Z)-(5-(1-(hydroxyimino)-3-methoxypropyl)pyridin-3-yl)carbamate (Step 4, 160 mg, 0.542 mmol) in EtOH (6 mL) was added Ni (160 mg, 2.73 mmol) and the mixture stirred at 50° C. for 2 h under H₂. The reaction mixture was filtered and the filtrate concentrated under reduced pressure to give the title compound as a yellow oil (150 mg, 98%). LCMS m/z=282 [M+H]⁺.

### Step 6. Synthesis of 5-(1-amino-3-methoxypropyl)pyridin-3-amine

A solution of tert-butyl(5-(1-amino-3-methoxypropyl)pyridin-3-yl)carbamate (Step 5, 150 mg, 0.533 mmol) in HCl/MeOH (2 mL) and MeOH (0.5 mL) was stirred at 25° C. for 30 min. The reaction mixture was concentrated under a stream of N₂ to give the title compound of a yellow oil (90 mg, 93%). LCMS m/z=182 [M+H]⁺.

### Step 7. Synthesis of tert-butyl(1-(5-aminopyridin-3-yl)-3-methoxypropyl)carbamate

Boc₂O (57.8 mg, 0.265 mmol) and NaHCO₃ (74.2 mg, 0.883 mmol) was added to a solution of 5-(1-amino-3-methoxypropyl)pyridin-3-amine (Step 6, 80 mg, 0.441 mmol) in dioxane (3 mL) and H₂O (1 mL) and the mixture stirred at 25° C. for 1 h. The reaction mixture was filtered and the filtrate concentrated under reduced pressure and the residue was purified by prep-HPLC-16 (3-35% MeCN) to give the title compound as a yellow oil (100 mg, 80%). LCMS m/z=282 [M+H]⁺.

### Intermediate A20. tert-butyl(R)-2-(3-aminophenyl) piperidine-1-carboxylate or tert-butyl(S)-2-(3-aminophenyl) piperidine-1-carboxylate

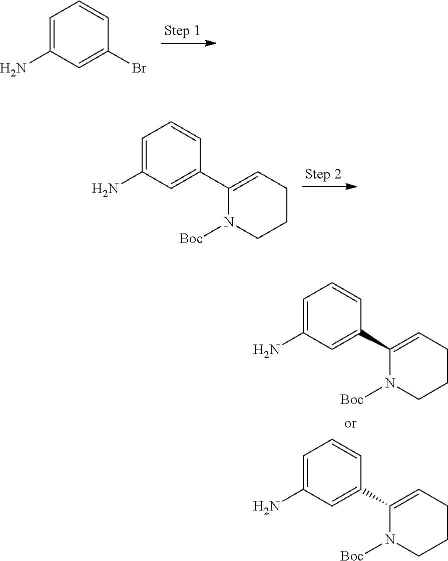

### Step 1. Synthesis of tert-butyl 6-(3-aminophenyl)-3,4-dihydropyridine-1 (2H)-carboxylate

To a solution of 3-bromoaniline (556 mg, 3.23 mmol) and tert-butyl 6-(4,4,5,5-tetramethyl-1,3,2-dioxaborolan-2-yl)-3,4-dihydropyridine-1 (2H)-carboxylate (1 g, 3.23 mmol) in H₂O (5 mL) and dioxane (20 mL) was added K₃PO₄ (1.37 g, 6.47 mmol) and XPHOS-PD-G2 (254 mg, 0.323 μmol) and the mixture stirred at 80° C. for 12 h under N₂. The reaction mixture was concentrated under reduced pressure and the residue was purified by MPLC (SiO₂, 1-25% EtOAc/PE) to give the title compound as a white solid (350 mg, 39%). LCMS m/z=275 [M+H]⁺.

Step 2. Synthesis of tert-butyl(R)-2-(3-aminophenyl) piperidine-1-carboxylate or tert-butyl(S)-2-(3-aminophenyl) piperidine-1-carboxylate.

To a solution of tert-butyl 6-(3-aminophenyl)-3,4-dihydropyridine-1 (2H)-carboxylate (Step 1, 330 mg, 1.20 mmol) in MeOH (10 mL) was added Pd/C (330 mg, 1.20 mmol, 10% purity) and the mixture stirred at 50° C. for 1 h under H₂ (50 psi). The reaction mixture was filtered and the filtrate concentrated under reduced pressure and the residue purified by prep-HPLC-17 (15-45% MeCN) followed by chiral-SFC (REGIS (s,s) WHELK-01, 250×50 mm, 10 μm); 50% IPA (0.1% NH₄OH) in CO₂) to afford:

Peak 2, Intermediate A20. tert-butyl(R)-2-(3-aminophenyl) piperidine-1-carboxylate or tert-butyl(S)-2-(3-aminophenyl) piperidine-1-carboxylate (yellow oil, 120 mg, 37%); LCMS m/z=277 [M+H]⁺.

### Intermediate A21. tert-butyl(R)-2-(5-amino-2-fluoropyridin-3-yl) piperidine-1-carboxylate or tert-butyl(S)-2-(5-amino-2-fluoropyridin-3-yl) piperidine-1-carboxylate

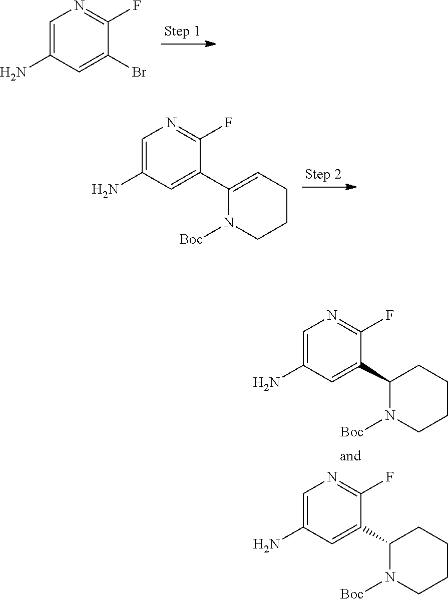 [SMILES: Nc1cnc(F)c(Br)c1 ; CC(C)(C)OC(=O)N1CCCC=C1c1cc(N)cnc1F ; CC(C)(C)OC(=O)N1CCCC[C@@H]1c1cc(N)cnc1F ; CC(C)(C)OC(=O)N1CCCC[C@H]1c1cc(N)cnc1F]

The title compounds were prepared from 5-bromo-6-fluoropyridin-3-amine and tert-butyl 6-(4,4,5,5-tetramethyl-1,3,2-dioxaborolan-2-yl)-3,4-dihydropyridine-1 (2H)-carboxylate using an analogous 2-step method as described for Intermediate A20. Prep-SFC (Diacel Chiralpak IC, 250×30 mm, 10 μm; 55% MeOH (0.1% NH₄OH) in CO₂.

Peak 1, Intermediate A21. tert-butyl(R)-2-(5-amino-2-fluoropyridin-3-yl) piperidine-1-carboxylate or tert-butyl(S)-2-(5-amino-2-fluoropyridin-3-yl) piperidine-1-carboxylate (white solid, 200 mg, 42%). LCMS m/z=296 [M+H]⁺.

### Intermediate A22. tert-butyl (R)-(1-(5-aminopyridin-3-yl)-2-methoxyethyl)carbamate or tert-butyl(S)-(1-(5-aminopyridin-3-yl)-2-methoxyethyl)carbamate

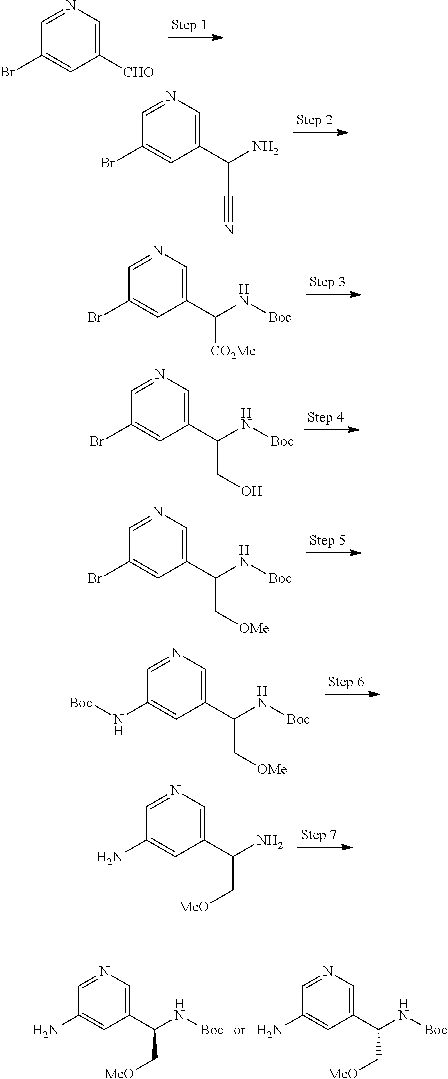

### Step 1. Synthesis of 2-amino-2-(5-bromopyridin-3-yl) acetonitrile

The reaction was carried out in triplicate.

Ti (OⁱPr)₄ (31.17 g, 110 mmol) and NH₃ (5 M, 235 mL) were added to a solution of 5-bromonicotinaldehyde (17 g, 91.39 mmol) in MeOH (100 mL) and the mixture stirred at 25° C. for 1 h and a solution of trimethylsilanecarbonitrile (10.88 g, 110 mmol) in MeOH (30 mL) added. The resulting mixture stirred at 25° C. for 12 h and poured into ice-water (250 mL) with stirring. The mixture was filtered and the foltrate extracted with EtOAc (3×250 mL). The combined organics were dried (Na₂SO₄) and concentrated under reduced pressure and the residue purified by column chromatography on silica gel (0-50% EtOAc/PE) to give the title compound as a yellow solid. The 3 batches were combined to afford (28.6 g, 49%). LCMS m/z=212 [M+H]⁺.

### Step 2. Synthesis of methyl 2-(5-bromopyridin-3-yl)-2-((tert-butoxycarbonyl)amino)acetate

A solution of 2-amino-2-(5-bromopyridin-3-yl) acetonitrile (Step 1, 11.64 g, 54.9 mmol) in HCl/MeOH (50 mL) was stirred at 25° C. for 12 h. The reaction mixture was filtered and the filtrate concentrated under reduced pressure to give methyl 2-amino-2-(5-bromopyridin-3-yl)acetate hydrochloride as a yellow solid (80.8 g, crude) which used without further purification. To a solution of methyl 2-amino-2-(5-bromopyridin-3-yl)acetate hydrochloride (80.8 g, 287 mmol) and Boc₂O (62.6 g, 287 mmol) in THF (450 mL) and H₂O (150 mL) was added NaHCO₃ (48.22 g, 574 mmol) and the mixture was stirred at 25° C. for 1 h. The reaction mixture was concentrated under reduced pressure and the residue diluted with H₂O (200 mL) and extracted with EtOAc (3×200 mL). The combined extracts were dried (Na₂SO₄) and concentrated under reduced pressure and the residue was purified by column chromatography on silica gel (0-50% EtOAc/PE) to give the title compound as a yellow oil (48 g, 48%). LCMS m/z=345 [M+H]⁺.

### Step 3. Synthesis of tert-butyl(1-(5-bromopyridin-3-yl)-2-hydroxyethyl)carbamate

LiCl (1.72 g, 40.7 mmol) was added to the mixture of NaBH₄ (1.54 g, 40.7 mmol) in EtOH (50 mL) and the mixture stirred at 0° C. for 10 min. To this was added a solution of methyl 2-(5-bromopyridin-3-yl)-2-((tert-butoxycarbonyl)amino)acetate (Step 2, 5.40 g, 15.64 mmol) in THF (50 mL) dropwise over 20 mins at 0° C. and the mixture stirred at 25° C. for 2 h. The reaction mixture was quenched by addition of H₂O (20 mL) at 0° C. and extracted with EtOAc (3×20 mL). The combined organics were dried (Na₂SO₄) and concentrated under reduced pressure and the residue purified by column chromatography on silica gel (0-50% EtOAc/PE) to give the title compound as a yellow oil (3.50 g, 70%). LCMS m/z=317 [M+H]⁺.

### Step 4. Synthesis of tert-butyl(1-(5-bromopyridin-3-yl)-2-methoxyethyl)carbamate

To a solution of tert-butyl(1-(5-bromopyridin-3-yl)-2-hydroxyethyl)carbamate (Step 3, 1.60 g, 5.04 mmol) in THF (15 mL) was added MeI (573 mg, 4.04 mmol) at 0° C. and the mixture stirred at 25° C. for 15 min. To this was added NaH (403.5 mg, 10.09 mmol, 60% purity) at 0° C. and stirred at 25° C. for 2 h. The mixture was quenched by addition of H₂O (10 mL) at 0° C. and extracted with EtOAc (3×10 mL). The combined organics were dried (Na₂SO₄) and concentrated under reduced pressure to give a residue which was purified by column chromatography on silica gel (1-50% EtOAc/PE) to give the title compound as a yellow oil (1.40 g, 42%). LCMS m/z=331 [M+H]⁺.

### Step 5. Synthesis of tert-butyl(1-(5-((tert-butoxycarbonyl)amino)pyridin-3-yl)-2-methoxyethyl)carbamate

To a solution of tert-butyl(1-(5-bromopyridin-3-yl)-2-methoxyethyl)carbamate (Step 4, 335 mg, 1.01 mmol) and tert-butyl carbamate (178 mg, 1.52 mmol) in dioxane (5 mL) was added Cs₂CO₃ (494 mg, 1.52 mmol), XPhos (48.22 mg, 0.101 mmol) and Pd₂ (dba)₃ (92.6 mg, 0.101 mmol) and the mixture was stirred at 100° C. for 2 h under N₂. The mixture was filtered and the filtrate was concentrated under reduced pressure and the residue purified by column chromatography on silica gel (1-50% EtOAc/PE) to give the title compound as a yellow oil (1.10 g, 99%). LCMS m/z=368 [M+H]⁺.

### Step 6. Synthesis of 5-(1-amino-2-methoxyethyl)pyridin-3-amine hydrochloride

A solution of tert-butyl(1-(5-((tert-butoxycarbonyl)amino)pyridin-3-yl)-2-methoxyethyl)carbamate (Step 5, 1.05 g, 2.86 mmol) in HCl/EtOAc (10 mL) was stirred at 25° C. for 1 h. The mixture was concentrated under reduced pressure to give the title compound as a yellow oil (500 mg, 86%). LCMS m/z=168 [M+H]⁺.

### Step 7. Synthesis of tert-butyl (R)-(1-(5-aminopyridin-3-yl)-2-methoxyethyl)carbamate or tert-butyl(S)-(1-(5-aminopyridin-3-yl)-2-methoxyethyl)carbamate

To a solution of 5-(1-amino-2-methoxyethyl)pyridin-3-amine hydrochloride (450 mg, 2.21 mmol) and (Boc)₂₀ (241.1 mg, 1.10 mmol) in H₂O (3 mL) and THF (15 mL) was added NaHCO₃ (371 mg, 4.42 mmol) and the mixture stirred at 25° C. for 1 h. The mixture was quenched with H₂O (10 mL) at 0° C. and extracted with EtOAc (3×10 mL). The combined extracts were evaporated to dryness and the residue purified by column chromatography on silica gel (1-33% EtOAc/PE) followed by chiral-SFC (Diacel Chiralpak IC, 250×30 mm, 10 μm; 10-50% EtOH (+0.1% NH₄OH) in CO₂) to afford:

Peak 1, Intermediate A22; tert-butyl (R)-(1-(5-aminopyridin-3-yl)-2-methoxyethyl)carbamate or tert-butyl(S)-(1-(5-aminopyridin-3-yl)-2-methoxyethyl)carbamate. LCMS m/z=268 [M+H]⁺.

### Intermediate A23. tert-butyl (R)-(1-(5-aminopyridin-3-yl)-2-methoxyethyl)(methyl)carbamate or tert-butyl(S)-(1-(5-aminopyridin-3-yl)-2-methoxyethyl)(methyl)carbamate

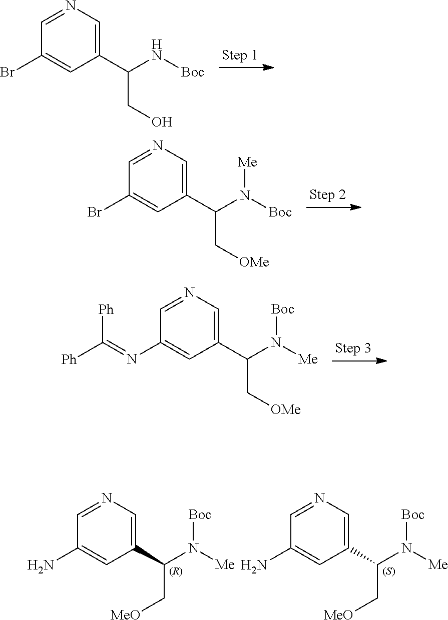

### Step 1. Synthesis of tert-butyl(1-(5-bromopyridin-3-yl)-2-methoxyethyl)(methyl)carbamate

To a solution of tert-butyl(1-(5-bromopyridin-3-yl)-2-hydroxyethyl)carbamate (Intermediate A22 Step 3, 5 g, 15.76 mmol) in THF (30 mL) was added NaH (2.52 g, 63 mmol, 60% purity) at 0° C. for 10 min followed by MeI (17.9 g, 126 mmol) and the mixture stirred at 25° C. for 2 h. The reaction mixture was quenched with sat·aq·NH₄Cl (20 ml) at 0° C. The mixture was diluted with H₂O (20 mL) and extracted with EtOAc (3×20 mL). The combined organics were dried (Na₂SO₄) and concentrated under reduced pressure and the residue purified by column chromatography on silica gel (0-25% EtOAc/PE) to give the title compound as a yellow oil (3 g, 55%). LCMS m/z=347 [M+H]⁺.

### Step 2. Synthesis of tert-butyl(1-(5-((diphenylmethylene)amino)pyridin-3-yl)-2-methoxyethyl)(methyl)carbamate

To a solution of tert-butyl(1-(5-bromopyridin-3-yl)-2-methoxyethyl)(methyl)carbamate (Step 1, 2.98 g, 8.63 mmol) and diphenylmethanimine (1.88 g, 10.4 mmol) in dioxane (30 mL) was added Cs₂CO₃ (5.62 g, 17.3 mmol), Xantphos (999 mg, 1.73 mmol) and Pd(dba)₂ (248 mg, 0.43 mmol) and the mixture stirred at 100° C. for 2 h under N₂. The reaction mixture was filtered and the filtrate evaporated to dryness under reduced pressure and extracted with EtOAc (3×30 mL). The combined organic layers were dried (Na₂SO₄) and concentrated under reduced pressure. The residue was purified by MPLC (SiO₂, 0-25% EtOAc/PE) to give the title compound as a yellow oil (2.40 g, 62%). LCMS m/z=446 [M+H]⁺.

### Step 3. tert-butyl (R)-(1-(5-aminopyridin-3-yl)-2-methoxyethyl)(methyl)carbamate and tert-butyl(S)-(1-(5-aminopyridin-3-yl)-2-methoxyethyl)(methyl)carbamate

To a solution of tert-butyl(1-(5-((diphenylmethylene)amino)pyridin-3-yl)-2-methoxyethyl)(methyl)carbamate (Step 2, 2.30 g, 5.16 mmol) in MeOH (25 mL) was added NH₂OH·HCl (430 mg, 6.19 mmol) and NaOAc (508 mg, 6.19 mmol) and the mixture stirred at 25° C. for 1 h. The reaction was concentrated under reduced pressure and the residue was extracted with EtOAc (3×30 mL). The combined organics were dried (Na₂SO₄), concentrated under reduced pressure and the residue purified by column chromatography on silica gel (0-100% EtOAc/PE) followed by prep-SFC (Phenomenex-Cellulose-2, 250×30 mm, 10 μm); 45% MeOH (0.1% NH₄OH) to afford:

Peak 1, Intermediate A23. tert-butyl (R)-(1-(5-aminopyridin-3-yl)-2-methoxyethyl)(methyl)carbamate or tert-butyl(S)-(1-(5-aminopyridin-3-yl)-2-methoxyethyl)(methyl)carbamate (yellow oil, 575 mg, 40%). LCMS m/z=282 [M+H]⁺.

### Intermediate A24. tert-butyl ((5-aminopyridin-3-yl)methyl)(ethyl)carbamate

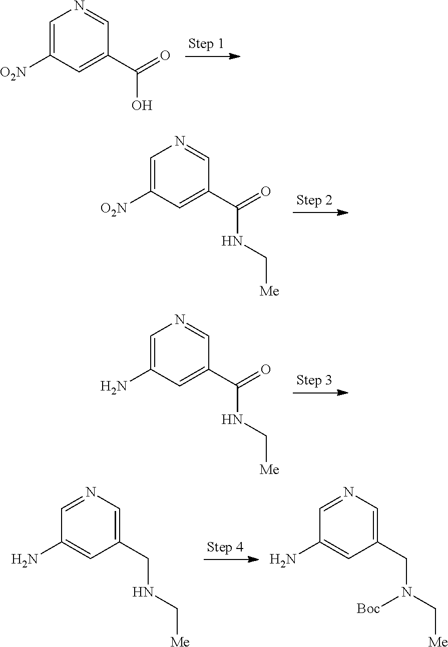 [SMILES: O=C(O)c1cncc([N+](=O)[O-])c1 ; CCNC(=O)c1cncc([N+](=O)[O-])c1 ; CCNC(=O)c1cncc(N)c1 ; CCNCc1cncc(N)c1 ; CCN(Cc1cncc(N)c1)C(=O)OC(C)(C)C]

### Step 1. Synthesis of N-ethyl-5-nitronicotinamide

To a mixture of 5-nitronicotinic acid (7 g, 41.6 mmol) and ethylamine hydrochloride (17 g, 208 mmol) in pyridine (60 mL) was added EDCI (12 g, 62.5 mmol) in one portion and the mixture was stirred at 25° C. for 4 h. The reaction mixture was filtered and the filtrate concentrated under reduced pressure and the reaction purified by column chromatography (SiO₂, 50% PE/EtOAc) to give the title compound as a yellow solid (6.11 g, 75%). LCMS m/z=196 [M+H]⁺.

### Step 2. Synthesis of 5-amino-N-ethylnicotinamide

To a solution of N-ethyl-5-nitronicotinamide (6 g, 30.74 mmol) in MeOH (100 mL) was slowly added Pd/C (6 g, 25.62 mmol, 10% purity) and the mixture stirred at 25° C. for 12 h under H₂ (15 psi). The reaction mixture was filtered and the filtrate concentrated under reduced pressure to give the title compound as a yellow solid (5 g, 98%). LCMS m/z=166 [M+H]⁺.

### Step 3. Synthesis of 5-((ethylamino)methyl)pyridin-3-amine

To 5-amino-N-ethylnicotinamide (4.5 g, 27.24 mmol, 1 eq) was added BH₃. THF (1M, 136.2 mL) at 0° C. under N₂ in one portion and the mixture stirred at 60° C. for 12 h. The reaction mixture was quenched by 0.1 N HCl (50 mL) at 0° C. and stirred at 80° C. for 1 h. The pH was adjusted to pH 6-7 by the addition of IN NH₄OH to pH 6-7 and the solids removed by filtration. The filtrate was evaporated to dryness to afford the title compound as a yellow oil (5.1 g, crude). LCMS m/z=152 [M+H]⁺.

### Step 4. Synthesis of tert-butyl ((5-aminopyridin-3-yl)methyl)(ethyl)carbamate

The mixture of 5-((ethylamino)methyl)pyridin-3-amine (5 g, 33 mmol) and di-tert-butyl dicarbonate in THF (15 mL) and H₂O (5 mL) was added NaHCO₃ (5.56 g, 66.1 mmol) and the mixture was stirred at 25° C. for 1 h. The reaction mixture was concentrated under reduced pressure and mixture diluted with H₂O (20 mL) and extracted with EtOAc (3×20 mL). The combined organics were dried (Na₂SO₄), evaporated to dryness under reduced pressure and the residue purified by column chromatography (SiO₂, 50% PE/EtOAc) to give the title compound as a yellow oil (3 g, 36%). LCMS m/z=252 [M+H]⁺.

### Intermediate A25. 5-((isopropylamino)methyl)pyridin-3-amine

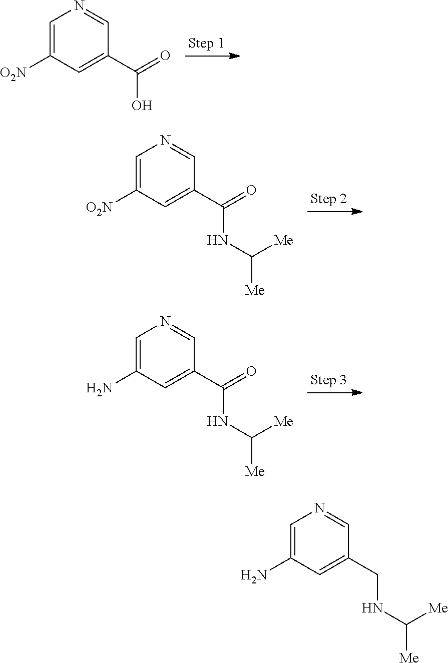

### Step 1. Synthesis of N-isopropyl-5-nitronicotinamide

To a mixture of 5-nitronicotinic acid (2 g, 11.9 mmol) and propan-2-amine (4.92 g, 83.3 mmol) in DMF (15 mL) was added HATU (6.79 g, 17.6 mmol) and DIPEA (2.31 g, 17.6 mmol) at 0° C. and the mixture stirred at 0° C. for 10 min and at 25° C. for 30 min. The solids were removed be filtration, the filtrate concentrated under reduced pressure and the residue purified by column chromatography on silica gel (0-50% EtOAc/PE) to give the title compound as a yellow solid (1.8 g, 72%). LCMS m/z=210 [M+H]⁺.

### Step 2. Synthesis of 5-amino-N-isopropylnicotinamide

To a solution of N-isopropyl-5-nitronicotinamide (Step 1, 1.5 g, 7.17 mmol) in MeOH (15 mL) was added Pd/C (1 g, 10% purity) and the mixture stirred under H₂ at 25° C. for 1 h. The reaction mixture was filtered and the filtrate concentrated under reduced pressure to give the title compound as a white solid (1 g, 78%). LCMS m/z=180 [M+H]⁺.

### Step 3. Synthesis of 5-((isopropylamino)methyl)pyridin-3-amine

To a solution of 5-amino-N-isopropylnicotinamide (Step 2, 800 mg, 4.46 mmol) was added BH₃. THF (1M, 22.32 mL) at 0° C. under N₂ and the mixture stirred at 60° C. for 2 h under N₂. The reaction mixture was quenched by 0.1 M HCl (50 mL) at 0° C., filtered and concentrated under reduced pressure. The residue was purified by prep-HPLC-11 (1-10% MeCN) to give the title compound as a yellow oil (100 mg, 13%). LCMS m/z=166 [M+H]⁺.

### Intermediate A26. (R)-5-(2-amino-1-methoxypropan-2-yl)pyridin-3-amine or (S)-5-(2-amino-1-methoxypropan-2-yl)pyridin-3-amine

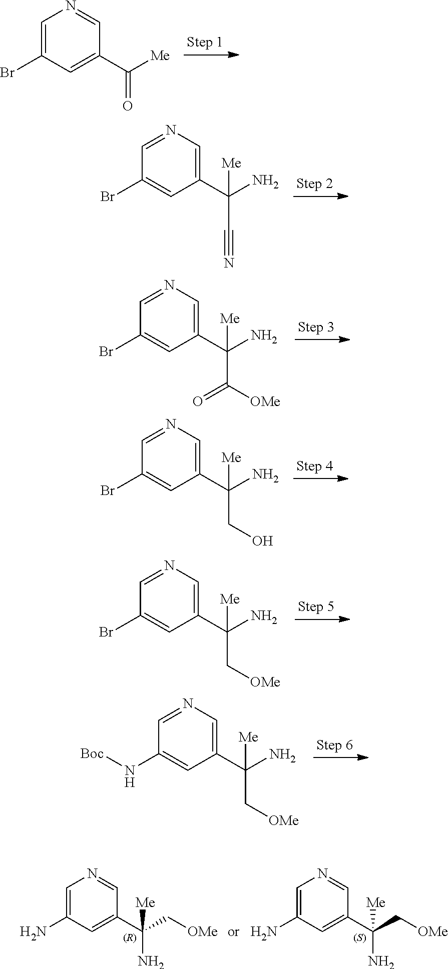

### Step 1. Synthesis of 2-amino-2-(5-bromopyridin-3-yl)propanenitrile

To a solution of 1-(5-bromopyridin-3-yl) ethan-1-one (10 g, 50 mmol) in MeOH (20 mL) was added tetraisopropoxytitanium (17.05 g, 60 mmol) and NH₃ (5M, 140 mL) and stirred at 25° C. for 1 h. To this was added then a solution of trimethylsilanecarbonitrile (5.95 g, 60 mmol) in MeOH (20 mL) and the mixture stirred at 25° C. for 12 h. The reaction mixture was poured into ice-water (50 mL) and the resulting mixture filtered through celite. The filtrate was extracted with EtOAc (3×50 mL) and the combined organics dried (Na₂SO₄) and concentrated under reduced pressure. The residue was purified by column chromatography on silica gel (15-50% EtOAc/PE) to give the title compound as a yellow solid (4.40 g, 39%). LCMS m/z=228 [M+H]⁺.

### Step 2. Synthesis of methyl 2-amino-2-(5-bromopyridin-3-yl)propanoate

A mixture of 2-amino-2-(5-bromopyridin-3-yl)propanenitrile (Step 1, 4.40 g, 19.5 mmol) in HCl/MeOH (50 mL) was stirred at 25° C. for 12 h. The reaction mixture was concentrated under reduced pressure to give the title compound as a brown oil (5.50 g, crude, HCl). LCMS m/z=259 [M+H]⁺.

### Step 3. Synthesis of 2-amino-2-(5-bromopyridin-3-yl)propan-1-ol

To a solution of NaBH₄ (759 mg, 20 mmol) in EtOH (50 mL) was added LiCl (851 mg, 20 mmol) and the mixture stirred at 0° C. for 10 min. A solution methyl 2-amino-2-(5-bromopyridin-3-yl)propanoate (Step 2, 2.00 g, 7.72 mmol) in THF (50 mL) was added dropwise over 20 min at 0° C. and the mixture warmed to 25° C. for 2 h. The reaction mixture was quenched by addition of H₂O (50 mL) at 0° C. and extracted with EtOAc (3×50 mL). The combined organics were dried (Na₂SO₄) and concentrated under reduced pressure. The residue was purified by column chromatography on silica gel (20% MeOH/EtOAc) to give the title compound as a yellow oil (1.50 g, 84%). LCMS m/z=231 [M+H]⁺.

### Step 4. Synthesis of 2-(5-bromopyridin-3-yl)-1-methoxypropan-2-amine

NaH (929 mg, 23.2 mmol, 60% purity) was added to a solution of 2-amino-2-(5-bromopyridin-3-yl)propan-1-ol (Part 3, 1.79 g, 7.75 mmol) in THF (20 mL) at 0° C. The mixture was stirred at 25° C. for 15 mins and MeI (880 mg, 6.20 mmol) added at 0° C. The mixture was stirred at 25° C. for 2 h, quenched by addition saturated aqueous NH₄Cl (10 mL) at 0° C. and extracted with EtOAc (3×10 mL). The combined organics were dried (Na₂SO₄) and concentrated under reduced pressure and the residue purified by column chromatography on silica gel (100% EtOAc) to give the title compound as a yellow oil (970 mg, 51%). LCMS m/z=245 [M+H]⁺.

### Step 5. Synthesis of tert-butyl(5-(2-amino-1-methoxypropan-2-yl)pyridin-3-yl)carbamate

To a solution of 2-(5-bromopyridin-3-yl)-1-methoxypropan-2-amine (Step 4, 940 mg, 3.83 mmol) and tert-butyl carbamate (674 mg, 5.75 mmol) in dioxane (10 mL) was added Cs₂CO₃ (1.87 g, 5.75 mmol) and Pd₂ (dba)₃ (351 mg, 0.38 mmol) and XPhos (183 mg, 0.38 mmol) and the mixture was stirred at 100° C. for 2 h under N₂. The reaction mixture was filtered and the filtrate concentrated under reduced pressure and the residue purified by column chromatography on silica gel (100% EtOAc) to give the title compound as a yellow oil (900 mg, 83%). LCMS m/z=282 [M+H]⁺.

### Step 6. Synthesis of (R)-5-(2-amino-1-methoxypropan-2-yl)pyridin-3-amine and (S)-5-(2-amino-1-methoxypropan-2-yl)pyridin-3-amine

A solution of tert-butyl(5-(2-amino-1-methoxypropan-2-yl)pyridin-3-yl)carbamate (Step 5, 880 mg, 3.13 mmol) in HCl/MeOH (5 mL) was stirred at 25° C. for 1 h. The reaction mixture was concentrated under reduced pressure and the pH of the residue adjusted pH=8 and purified by prep-SFC (DAICEL CHIRALPAK IC, 250×30 mm, 10 μm; 50% IPA (0.1% NH₄OH) in CO₂) to afford:

Peak 1, Intermediate A26. (R)-5-(2-amino-1-methoxypropan-2-yl)pyridin-3-amine or (S)-5-(2-amino-1-methoxypropan-2-yl)pyridin-3-amine (200 mg, 35%); LCMS m/z=182 [M+H]⁺.

### Intermediate A27. tert-butyl(R)-3-(5-aminopyridin-3-yl)-1,4-oxazepane-4-carboxylate or tert-butyl(S)-3-(5-aminopyridin-3-yl)-1,4-oxazepane-4-carboxylate

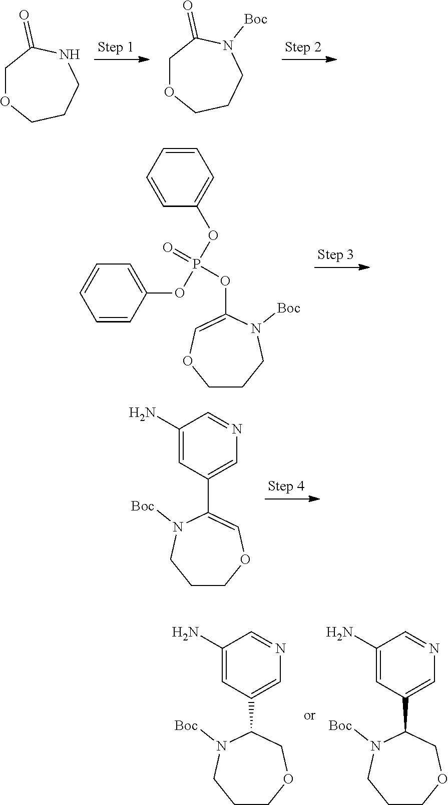

### Step 1. Synthesis of tert-butyl 3-oxo-1,4-oxazepane-4-carboxylate

To a solution of 1,4-oxazepan-3-one (1.94 g, 16.9 mmol) and (Boc)₂₀ (11 g, 50.6 mmol) in DCM (30 mL) was added DMAP (206 mg, 1.69 mmol) and TEA (2.56 g, 25.3 mmol) and the mixture stirred at 40° C. for 12 h under N₂. The reaction mixture was diluted with H₂O (60 mL) and extracted with EtOAc (3×40 mL). The combined organics were washed with brine (2×30 mL), dried (Na₂SO₄) and concentrated under reduced pressure and the residue purified by MPLC (SiO₂, 0-25% EtOAc/PE) to give the title compound as a brown oil (2.52 g, 69%). LCMS m/z=216 [M+H]⁺.

### Step 2. Synthesis of tert-butyl 3-((diphenoxyphosphoryl)oxy)-6, 7-dihydro-1,4-oxazepine-4 (5H)-carboxylate

To a solution of tert-butyl 3-oxo-1,4-oxazepane-4-carboxylate (Step 1, 1.5 g, 6.97 mmol) in THF (25 mL) was added KHMDS (1M, 10.45 mL) at −78° C. for 1.5 h and diphenyl phosphorochloridate (2.81 g, 10.45 mmol) added. The mixture was stirred at 0° C. for 1 h under N₂. The reaction mixture was diluted with H₂O (80 mL) and extracted with EtOAc (3×50 mL). The combined organics were washed with brine (2×30 mL), dried (Na₂SO₄) and concentrated under reduced pressure and the residue purified by MPLC (SiO₂, 0-25% EtOAc/PE) to give the title compound as a yellow oil (1.46 g, 46%). LCMS m/z=448 [M+H].

### Step 3. Synthesis of tert-butyl 3-(5-aminopyridin-3-yl)-6,7-dihydro-1,4-oxazepine-4 (5H)-carboxylate

To a solution of tert-butyl 3-((diphenoxyphosphoryl)oxy)-6,7-dihydro-1,4-oxazepine-4 (5H)-carboxylate (Step 2, 1.32 g, 2.94 mmol) and 5-(4,4,5,5-tetramethyl-1,3,2-dioxaborolan-2-yl)pyridin-3-amine (970 mg, 4.41 mmol) in DME (15 mL) was added Pd(PPh₃)₄ (170 mg, 0.147 mmol) and Na₂CO₃ (2M, 2.94 mL) and the mixture stirred at 90° C. for 2 h under N₂.

The reaction mixture was concentrated under reduced pressure and the residue purified by MPLC (SiO₂, 0-50% EtOA/PE) to give the title compound as a brown solid (340 mg, 40%). LCMS m/z=292 [M+H]⁺.

### Step 4. Synthesis of tert-butyl(R)-3-(5-aminopyridin-3-yl)-1,4-oxazepane-4-carboxylate or tert-butyl(S)-3-(5-aminopyridin-3-yl)-1,4-oxazepane-4-carboxylate

A mixture of tert-butyl 3-(5-aminopyridin-3-yl)-6,7-dihydro-1,4-oxazepine-4 (5H)-carboxylate (Step 3, 320 mg, 1.10 mmol) and Pd/C (400 mg, 10% purity) in MeOH (5 mL) was stirred at 50° C. for 2 h under H₂ (50 psi). The reaction mixture was filtered and the filtrate was concentrated under reduced pressure to give tert-butyl 3-(5-aminopyridin-3-yl)-1,4-oxazepane-4-carboxylate as a white solid (190 mg). tert-butyl 3-(5-aminopyridin-3-yl)-1,4-oxazepane-4-carboxylate was separated by chiral-SFC (DAICEL CHIRALPAK AD, 250×30 mm, 10 μm); 20% MeOH (0.1% NH₄OH) in CO₂) to afford:

Peak 1, Intermediate A27. tert-butyl(R)-3-(5-aminopyridin-3-yl)-1,4-oxazepane-4-carboxylate or tert-butyl(S)-3-(5-aminopyridin-3-yl)-1,4-oxazepane-4-carboxylate (brown oil, 92 mg, 28%); LCMS m/z=294 [M+H]⁺.

### Intermediate A28. tert-butyl 5-(5-aminopyridin-3-yl)-1,4-oxazepane-4-carboxylate

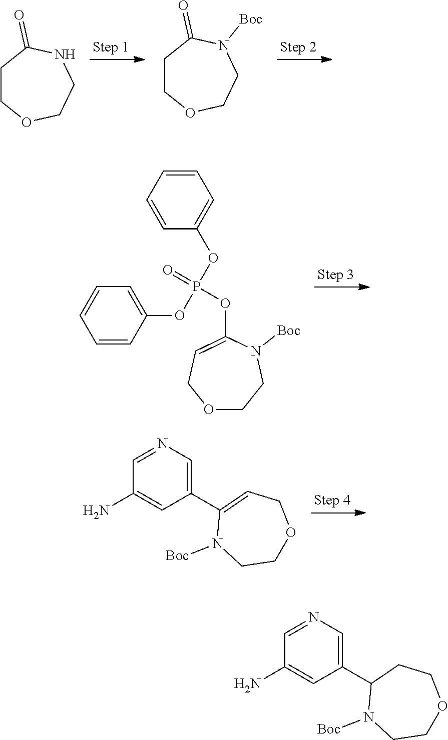 [SMILES: O=C1CCOCCN1 ; CC(C)(C)OC(=O)N1CCOCCC1=O ; CC(C)(C)OC(=O)N1CCOCC=C1OP(=O)(Oc1ccccc1)Oc1ccccc1 ; CC(C)(C)OC(=O)N1CCOCC=C1c1cncc(N)c1 ; CC(C)(C)OC(=O)N1CCOCCC1c1cncc(N)c1]

The title compound was prepared from 1,4-oxazepan-5-one using an analogous 4-Step method to that described for Intermediate A27 without chiral-SFC purification. LCMS m/z=294 [M+H]⁺.

### Intermediate A29 and A30. tert-butyl(R)-2-(5-aminopyridin-3-yl) azepane-1-carboxylate and tert-butyl(S)-2-(5-aminopyridin-3-yl) azepane-1-carboxylate

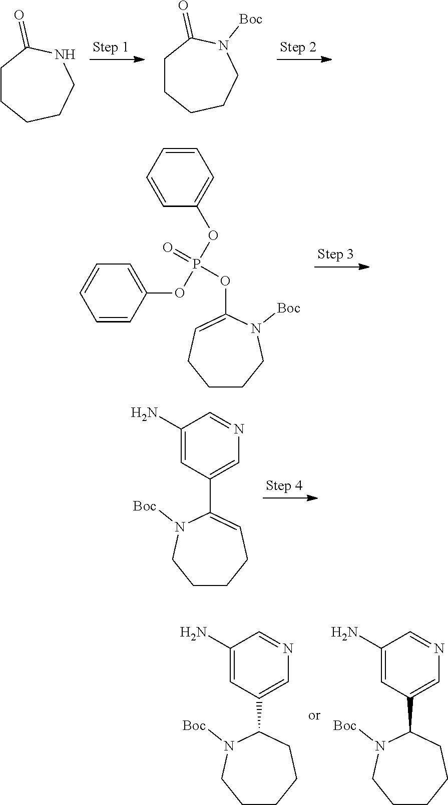

The title compounds were prepared from azepan-2-one using an analogous 4 Step method as described for Intermediate A27. Chiral-SFC (REGIS (s,s) WHELK-01, 250×50 mm, 10 μm); 40% EtOH (0.1% NH₄OH) in CO₂) afforded:

Peak 1, Intermediate A29. tert-butyl(R)-2-(5-aminopyridin-3-yl) azepane-1-carboxylate or tert-butyl(S)-2-(5-aminopyridin-3-yl) azepane-1-carboxylate (white solid, 1.35 g, 42%); LCMS m/z=292 [M+H]⁺.

Peak 2, Intermediate A30. tert-butyl(R)-2-(5-aminopyridin-3-yl) azepane-1-carboxylate or tert-butyl(S)-2-(5-aminopyridin-3-yl) azepane-1-carboxylate (white solid, 1.5 g, 46%); LCMS m/z=292 [M+H]⁺.

### Intermediate A31, A32, A33 and A34. tert-butyl(2S,6R)-2-(5-aminopyridin-3-yl)-6-methylpiperidine-1-carboxylate and tert-butyl(2S,6S)-2-(5-aminopyridin-3-yl)-6-methylpiperidine-1-carboxylate and tert-butyl(2R,6S)-2-(5-aminopyridin-3-yl)-6-methylpiperidine-1-carboxylate and tert-butyl(2R,6R)-2-(5-aminopyridin-3-yl)-6-methylpiperidine-1-carboxylate

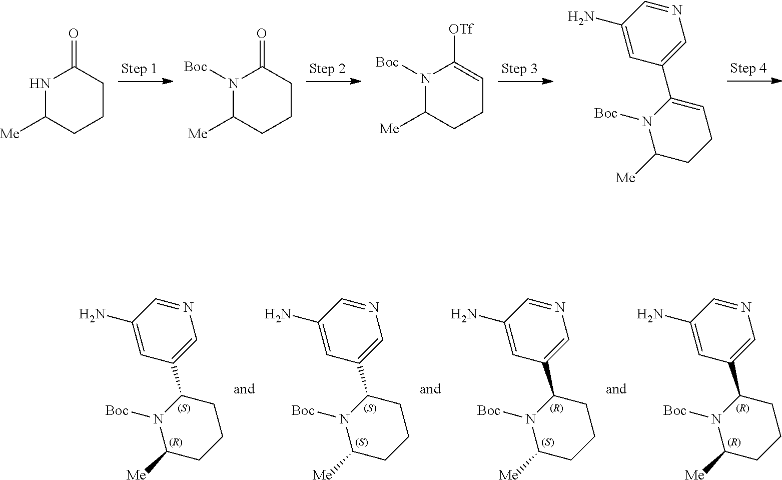

### Step 1. Synthesis of tert-butyl 2-methyl-6-oxopiperidine-1-carboxylate

LDA (2M, 33.1 mL) was added to a solution of 6-methylpiperidin-2-one (6.8 g, 60.1 mmol) in THF (60 mL) at −78° C. for 30 min. To this was added (Boc)₂₀ (14.43 g, 66.1 mmol) and the mixture stirred at −78° C. for 2 h under N₂. The reaction mixture was quenched by addition saturated aqueous NH₄Cl solution (100 mL) at 0° C. and extracted with EtOAc (3×100 mL). The combined organics were dried (Na₂SO₄) and concentrated under reduced pressure to give the title compound as a yellow oil (12 g, 93%).

### Step 2. Synthesis of tert-butyl 2-methyl-6-(((trifluoromethyl) sulfonyl)oxy)-3,4-dihydropyridine-1 (2H)-carboxylate

LDA (2M, 35.17 mL) was added to a solution of tert-butyl 2-methyl-6-oxopiperidine-1-carboxylate (Step 1, 10 g, 46.9 mmol) in THF (50 mL) at −78° C. under N₂ and the mixture stirred at −78° C. for 0.5 h. To this was added dropwise a solution of N-(5-chloro-2-pyridyl)-1,1,1-trifluoro-N-(trifluoromethylsulfonyl) methanesulfonamide (20.25 g, 51.6 mmol) in THF and the mixture stirred at 25° C. for 2 h under N₂. The reaction mixture was quenched by addition saturated aqueous NH₄Cl solution (100 mL) at 0° C., and then extracted with EtOAc (3×100 mL). The combined organics were dried (Na₂SO₄) and concentrated under reduced pressure and the residue purified by column chromatography (SiO₂, 0-5% EtOAc/PE) to give the title compound as a yellow oil (11 g, 68%).

### Step 3. Synthesis of tert-butyl 5′-amino-6-methyl-5,6-dihydro-[2,3′-bipyridine]-1 (4H)-carboxylate

To a mixture of tert-butyl 2-methyl-6-(((trifluoromethyl) sulfonyl)oxy)-3,4-dihydropyridine-1 (2H)-carboxylate (Step 2, 10.5 g, 30.41 mmol) and 5-(4,4,5,5-tetramethyl-1,3,2-dioxaborolan-2-yl)pyridin-3-amine (7.03 g, 31.93 mmol) in dioxane (100 mL) and H₂O (10 mL) was added Pd(PPh₃)₄ (1.05 g, 0.912 mmol) and Cs₂CO₃ (19.81 g, 60.81 mmol) and the mixture stirred at 110° C. for 1 h under N₂. The reaction mixture was filtered and the filtrate concentrated under reduced pressure and the residue purified by column chromatography (SiO₂, 10-100% EtOAc/PE) to give the title compound as a yellow oil (5.5 g, 62%). LCMS m/z=290 [M+H]⁺.

### Step 4. Synthesis of tert-butyl(2S,6R)-2-(5-aminopyridin-3-yl)-6-methylpiperidine-1-carboxylate and tert-butyl(2S,6S)-2-(5-aminopyridin-3-yl)-6-methylpiperidine-1-carboxylate and tert-butyl(2R,6S)-2-(5-aminopyridin-3-yl)-6-methylpiperidine-1-carboxylate and tert-butyl(2R,6R)-2-(5-aminopyridin-3-yl)-6-methylpiperidine-1-carboxylate

To a solution of tert-butyl 5′-amino-6-methyl-5,6-dihydro-[2,3′-bipyridine]-1 (4H)-carboxylate (Step 3, 2 g, 6.91 mmol) in MeOH (40 mL) was added Pd/C (2 g, 6.91 mmol, 10% purity) and the mixture stirred under H₂ at 50° C. for 1 h. The reaction mixture was filtered and the filtrate concentrated under reduced pressure to give tert-butyl 2-(5-aminopyridin-3-yl)-6-methylpiperidine-1-carboxylate as a white solid (1.3 g, 64%) which was purified by chiral-SFC (REGIS (S,S) WHELK-01, 250×25 mm, 10 μm); 30% IPA (0.1% NH₄OH) in CO₂) to afford:

Peak 1, Intermediate A31. tert-butyl(2S,6R)-2-(5-aminopyridin-3-yl)-6-methylpiperidine-1-carboxylate or tert-butyl(2S,6S)-2-(5-aminopyridin-3-yl)-6-methylpiperidine-1-carboxylate or tert-butyl(2R,6S)-2-(5-aminopyridin-3-yl)-6-methylpiperidine-1-carboxylate or tert-butyl(2R,6R)-2-(5-aminopyridin-3-yl)-6-methylpiperidine-1-carboxylate (white solid, 580 mg). LCMS m/z=292 [M+H]⁺.

Peak 2, Intermediate A32. tert-butyl(2S,6R)-2-(5-aminopyridin-3-yl)-6-methylpiperidine-1-carboxylate or tert-butyl(2S,6S)-2-(5-aminopyridin-3-yl)-6-methylpiperidine-1-carboxylate or tert-butyl(2R,6S)-2-(5-aminopyridin-3-yl)-6-methylpiperidine-1-carboxylate or tert-butyl(2R,6R)-2-(5-aminopyridin-3-yl)-6-methylpiperidine-1-carboxylate (white solid, 280 mg). LCMS m/z=292 [M+H]⁺.

Peak 3, Intermediate A33. tert-butyl(2S,6R)-2-(5-aminopyridin-3-yl)-6-methylpiperidine-1-carboxylate or tert-butyl(2S,6S)-2-(5-aminopyridin-3-yl)-6-methylpiperidine-1-carboxylate or tert-butyl(2R,6S)-2-(5-aminopyridin-3-yl)-6-methylpiperidine-1-carboxylate or tert-butyl(2R,6R)-2-(5-aminopyridin-3-yl)-6-methylpiperidine-1-carboxylate (yellow oil, 600 mg). LCMS m/z=292 [M+H]⁺.

Peak 4, Intermediate A34. tert-butyl(2S,6R)-2-(5-aminopyridin-3-yl)-6-methylpiperidine-1-carboxylate or tert-butyl(2S,6S)-2-(5-aminopyridin-3-yl)-6-methylpiperidine-1-carboxylate or tert-butyl(2R,6S)-2-(5-aminopyridin-3-yl)-6-methylpiperidine-1-carboxylate or tert-butyl(2R,6R)-2-(5-aminopyridin-3-yl)-6-methylpiperidine-1-carboxylate (yellow oil, 300 mg). LCMS m/z=292 [M+H]⁺.

### Intermediate A35, A36, A37 and A38. tert-butyl(2R,5R)-2-(5-aminopyridin-3-yl)-5-methylpyrrolidine-1-carboxylate and tert-butyl(2S,5S)-2-(5-aminopyridin-3-yl)-5-methylpyrrolidine-1-carboxylate and tert-butyl(2R,5S)-2-(5-aminopyridin-3-yl)-5-methylpyrrolidine-1-carboxylate and tert-butyl(2S,5R)-2-(5-aminopyridin-3-yl)-5-methylpyrrolidine-1-carboxylate

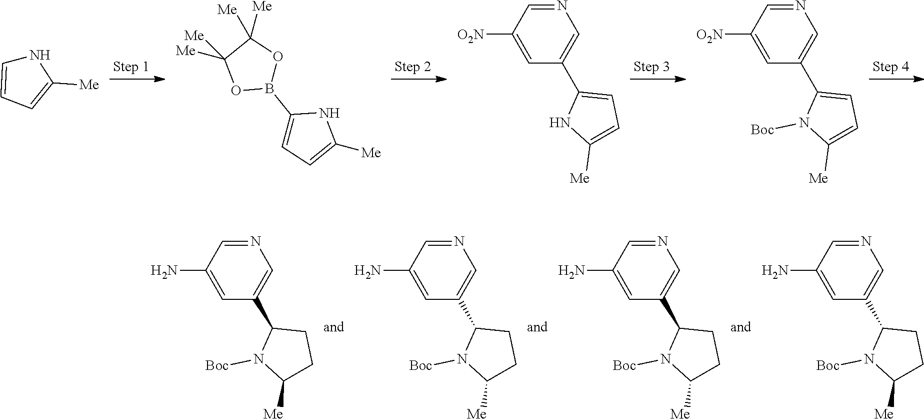

### Step 1. Synthesis of 2-methyl-5-(4,4,5,5-tetramethyl-1,3,2-dioxaborolan-2-yl)-1H-pyrrole

A mixture of 2-methylpyrrole (8.00 g, 98.6 mmol), 4,4,5,5-tetramethyl-2-(4,4,5,5-tetramethyl-1,3,2-dioxaborolan-2-yl)-1,3,2-dioxaborolane (12.52 g, 49.3 mmol), 4,4′-di-tert-butyl-2,2′-bipyridine (1.06 g, 3.94 mmol) and (1,5-Cyclooctadiene)(methoxy) iridium (I) dimer (1.31 g, 1.97 mmol) in hexane (80 mL) was degassed and purged with N₂ (×3) and stirred at 30° C. for 2 h under N₂. The reaction mixture was concentrated under reduced pressure and the residue purified by column chromatography (SiO₂, 0-10% EtOAc/PE) to give the title compound as a yellow solid (8.1 g, 40%). LCMS m/z=208 [M+H]⁺.

### Step 2. Synthesis of 3-(5-methyl-1H-pyrrol-2-yl)-5-nitropyridine

To a solution of 2-methyl-5-(4,4,5,5-tetramethyl-1,3,2-dioxaborolan-2-yl)-1H-pyrrole (Part 1, 8.08 g, 39 mmol) in dioxane (40 mL) and H₂O (4 mL) was added 3-bromo-5-nitro-pyridine (5.50 g, 27.1 mmol), K₂CO₃ (7.49 g, 54.2 mmol) and Pd(PPh₃)₄ (3.13 g, 2.71 mmol) and the mixture stirred at 110° C. for 3 h. The reaction mixture was filtered and the filtrate concentrated under reduced pressure and the residue diluted with H₂O (50 mL) and extracted with EtOAc (3×40 mL). The combined organics were washed with brine (2×30 mL), dried (Na₂SO₄) and concentrated under reduced pressure. The residue was purified by column chromatography (SiO₂, 0-50% EtOAc/PE/EtOAc) to give the title compound as a yellow solid (8.40 g). LCMS m/z=204 [M+H]⁺.

### Step 3. Synthesis of tert-butyl 2-methyl-5-(5-nitropyridin-3-yl)-1H-pyrrole-1-carboxylate

To a solution of 3-(5-methyl-1H-pyrrol-2-yl)-5-nitropyridine (Part 2, 8.2 g, 40.4 mmol) in DCM (50 mL) was added DMAP (245 mg, 2.02 mmol) and di-tert-butyl dicarbonate (9.26 g, 42.42 mmol) and the mixture stirred at 40° C. for 16 h. The reaction mixture was concentrated under reduced pressure and the residue diluted with H₂O (50 mL0 and extracted with EtOAc (3×40 mL). The combined organics were washed with brine (2×30 mL), dried (Na₂SO₄) and concentrated under reduced pressure. The residue was purified by column chromatography (SiO₂, 0-20% EtOAc/PE) to give the title compound as a yellow solid (7.50 g, 61%). LCMS m/z=304 [M+H]⁺.

### Step 4. Synthesis of tert-butyl(2R,5R)-2-(5-aminopyridin-3-yl)-5-methylpyrrolidine-1-carboxylate and tert-butyl(2S,5S)-2-(5-aminopyridin-3-yl)-5-methylpyrrolidine-1-carboxylate and tert-butyl(2R,5S)-2-(5-aminopyridin-3-yl)-5-methylpyrrolidine-1-carboxylate and tert-butyl(2S,5R)-2-(5-aminopyridin-3-yl)-5-methylpyrrolidine-1-carboxylate

To a solution of tert-butyl 2-methyl-5-(5-nitropyridin-3-yl)-1H-pyrrole-1-carboxylate (Step 3, 7 g, 23.1 mmol) in EtOH (100 mL) was added Pd/C (7 g, 10% purity) and the mixture stirred under H₂ at 60° C. for 16 h. The reaction mixture was filtered and the filtrate concentrated under reduced pressure to give tert-butyl 2-(5-aminopyridin-3-yl)-5-methylpyrrolidine-1-carboxylate as a white solid (6 g) which was purified by chiral-SFC (REGIS (S,S) WHELK-O1, 250×25 mm, 10 μm); 30% IPA (0.1% NH₄OH) in CO₂) to afford:

Peak 1, Intermediate A35. tert-butyl(2R,5R)-2-(5-aminopyridin-3-yl)-5-methylpyrrolidine-1-carboxylate or tert-butyl(2S,5S)-2-(5-aminopyridin-3-yl)-5-methylpyrrolidine-1-carboxylate or tert-butyl(2R,5S)-2-(5-aminopyridin-3-yl)-5-methylpyrrolidine-1-carboxylate or tert-butyl(2S,5R)-2-(5-aminopyridin-3-yl)-5-methylpyrrolidine-1-carboxylate (white solid, 1.35 g). LCMS m/z=278 [M+H]⁺.

Peak 2, Intermediate A36. tert-butyl(2R,5R)-2-(5-aminopyridin-3-yl)-5-methylpyrrolidine-1-carboxylate or tert-butyl(2S,5S)-2-(5-aminopyridin-3-yl)-5-methylpyrrolidine-1-carboxylate or tert-butyl(2R,5S)-2-(5-aminopyridin-3-yl)-5-methylpyrrolidine-1-carboxylate or tert-butyl(2S,5R)-2-(5-aminopyridin-3-yl)-5-methylpyrrolidine-1-carboxylate (white solid, 1.5 g). LCMS m/z=278 [M+H]⁺.

Peak 3, Intermediate A37. tert-butyl(2R,5R)-2-(5-aminopyridin-3-yl)-5-methylpyrrolidine-1-carboxylate or tert-butyl(2S,5S)-2-(5-aminopyridin-3-yl)-5-methylpyrrolidine-1-carboxylate or tert-butyl(2R,5S)-2-(5-aminopyridin-3-yl)-5-methylpyrrolidine-1-carboxylate or tert-butyl(2S,5R)-2-(5-aminopyridin-3-yl)-5-methylpyrrolidine-1-carboxylate (white solid, 1.5 g). LCMS m/z=278 [M+H]⁺.

Peak 4, Intermediate A38. tert-butyl(2R,5R)-2-(5-aminopyridin-3-yl)-5-methylpyrrolidine-1-carboxylate or tert-butyl(2S,5S)-2-(5-aminopyridin-3-yl)-5-methylpyrrolidine-1-carboxylate or tert-butyl(2R,5S)-2-(5-aminopyridin-3-yl)-5-methylpyrrolidine-1-carboxylate or tert-butyl(2S,5R)-2-(5-aminopyridin-3-yl)-5-methylpyrrolidine-1-carboxylate (white solid, 1.35 g). LCMS m/z=278 [M+H]⁺.

### Intermediate A39. 5-(1-aminocyclopentyl)pyridin-3-amine

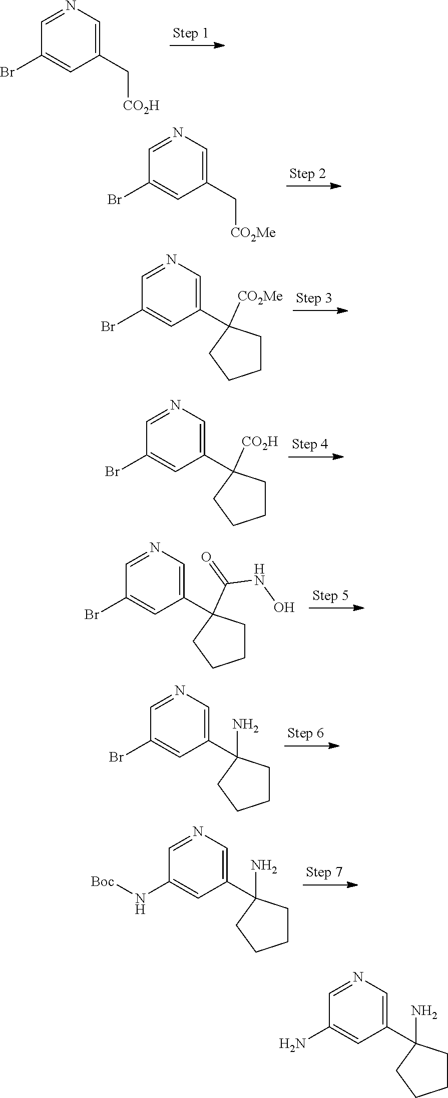

### Step 1. Synthesis of methyl 2-(5-bromopyridin-3-yl)acetate

To a solution of 2-(5-bromopyridin-3-yl) acetic acid (5 g, 23.14 mmol) in MeOH (40 mL) was added SOCl₂ (4 mL) and the mixture stirred at 60° C. for 1 h. The reaction mixture was concentrated under reduced pressure and the residue was diluted with H₂O (10 mL) and extracted with EtOAc (3×10 mL). The combined organics were concentrated under reduced pressure and the residue purified by MPLC (SiO₂, 1-5% EtOAc/PE) to give the title compound as a white oil (5.26 g, 98%). LCMS m/z=230 [M+H]⁺.

### Step 2. Synthesis of methyl 1-(5-bromopyridin-3-yl)cyclopentane-1-carboxylate

To a solution of methyl 2-(5-bromopyridin-3-yl)acetate (Step 1, 1.5 g, 6.52 mmol) and 1,4-diiodobutane (2.02 g, 6.52 mmol) in THF (5 mL) was added LiHMDS (1M, 8.15 mL) at 0° C. and the mixture stirred at 25° C. for 2 h under N₂. To this was added LiHMDS (1M, 8.15 mL) at 0° C. and the mixture stirred at 25° C. for 2 h under N₂. The reaction mixture was quenched by addition saturated aqueous NH₄Cl (2 mL) at 0° C. and diluted with H₂O (2 mL) and extracted with EtOAc (3×2 mL). The combined organics were dried (Na₂SO₄) and concentrated under reduced pressure. The residue was purified by MPLC (SiO₂, 1-5% EtOAc/PE) to give the title compound as a brown oil (1.32 g, 71%) as. LCMS m/z=284 [M+H]⁺.

### Step 3. Synthesis of 1-(5-bromopyridin-3-yl)cyclopentane-1-carboxylic acid

To a solution of methyl 1-(5-bromopyridin-3-yl)cyclopentane-1-carboxylate (Step 2, 1.32 g, 4.65 mmol) in THF (10 mL) and H₂O (5 mL) was added LiOH (222 mg, 9.29 mmol) and the mixture stirred at 40° C. for 4 h. The reaction mixture was concentrated under reduced pressure and the pH adjusted with 1M HCl to pH 4-5, diluted with H₂O (20 mL) and extracted with EtOAc (3×20 mL). The combined organics were dried (Na₂SO₄) and concentrated under reduced pressure to give the title compound as a red solid (1.09 g, 87%). LCMS m/z=270 [M+H]⁺.

### Step 4. Synthesis of 1-(5-bromopyridin-3-yl)-N-hydroxycyclopentane-1-carboxamide

To a solution of 1-(5-bromopyridin-3-yl)cyclopentane-1-carboxylic acid (Step 3, 1.36 g, 5.03 mmol) and hydroxylamine hydrochloride (420 mg, 6.04 mmol) and DIPEA (1.95 g, 15.1 mmol) in DMF (1 mL) was added BOP reagent (3.34 g, 7.55 mmol) and the mixture stirred at 25° C. for 0.5 h. The reaction mixture was quenched by addition H₂O (3 mL) at 0° C. and extracted with EtOAc (3×5 mL). The combined organics were dried (Na₂SO₄), concentrated under reduced pressure and the residue purified by prep-HPLC-15 (5-35% MeCN) to give the title compound as a white solid (216 mg, 15%). LCMS m/z=285 [M+H]⁺.

### Step 5. Synthesis of 1-(5-bromopyridin-3-yl)cyclopentan-1-amine

To a solution of 1-(5-bromopyridin-3-yl)-N-hydroxycyclopentane-1-carboxamide (Step 4, 195 mg, 0.684 mmol) in DMSO (3 mL) was added K₂CO₃ (472 mg, 3.42 mmol) and the mixture stirred at 80° C. for 12 h. The reaction mixture was extracted with H₂O (10 mL) and EtOAc (3×10 mL). The combined organics were concentrated under reduced pressure and the residue purified by prep-TLC (SiO₂, 10% MeOH/EtOAc) to give the title compound as a yellow oil (113 mg, 68%). LCMS m/z=241 [M+H]⁺.

### Step 6. Synthesis of tert-butyl(5-(1-aminocyclopentyl)pyridin-3-yl)carbamate

To a solution of 1-(5-bromopyridin-3-yl)cyclopentan-1-amine (Step 5, 100 mg, 0.415 mmol) and tert-butyl carbamate (72.9 mg, 0.622 mmol) in dioxane (5 mL) was added Pd₂ (dba)₃ (38 mg, 0.415 mmol), XPhos (19.77 mg, 0. 415 mmol) and Cs₂CO₃ (203 mg, 0.622 mmol) and the mixture was stirred at 100° C. for 2 h under N₂. The reaction mixture was concentrated under reduced pressure to give a residue and the residue purified by prep-TLC (EtOAc) to give the title compound as a yellow solid (105 mg, 91%). LCMS m/z=278 [M+H]⁺. Step 7. Synthesis of 5-(1-aminocyclopentyl)pyridin-3-amine.

To a solution of tert-butyl(5-(1-aminocyclopentyl)pyridin-3-yl)carbamate (Part 6, 105 mg, 0.378 mmol) in HCl/EtOAc (2 mL) and the mixture stirred at 25° C. for 1 h. The reaction mixture was concentrated under reduced pressure and the residue adjusted with DIPEA to pH 8-9 and purified by prep-HPLC-13 (1-15% MeCN) to give the title compound as a white solid (43 mg, 64%). LCMS m/z=178 [M+H]⁺.

### Intermediate A40. tert-butyl(1-(5-aminopyridin-3-yl)cyclopropyl)carbamate

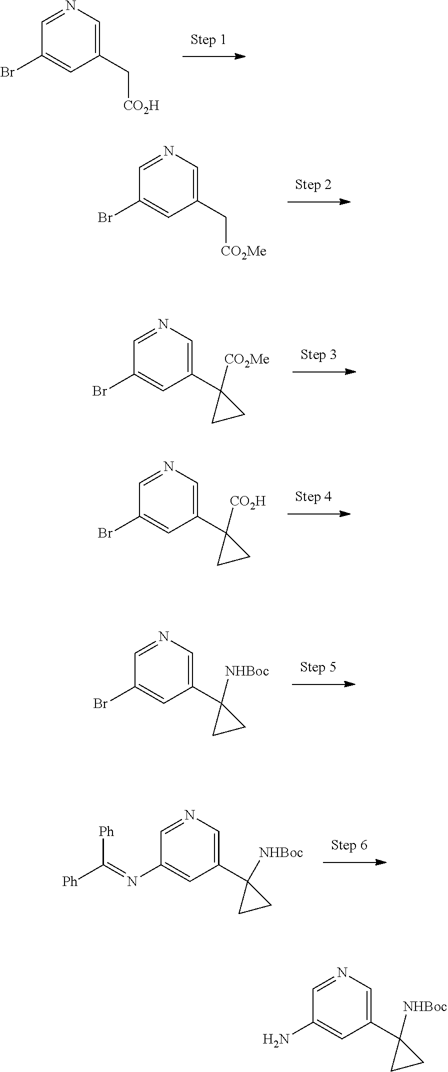

### Step 1-3. Synthesis of 1-(5-bromopyridin-3-yl)cyclopropane-1-carboxylic acid

The title compound was prepared using an analogous 3-Step method as described for 1-(5-bromopyridin-3-yl)cyclopentane-1-carboxylic acid (Intermediate A39 Steps 1-3). LCMS: m/z=242 [M+H]⁺.

### Step 4. Synthesis of tert-butyl(1-(5-bromopyridin-3-yl)cyclopropyl)carbamate

To a solution of 1-(5-bromopyridin-3-yl)cyclopropane-1-carboxylic acid (Step 3, 600 mg, 2.48 mmol) in t-BuOH (24 mL) was added DPPA (1.02 g, 3.72 mmol), 4 Å molecular sieve (600 mg) and triethylamine (376 mg, 3.72 mmol). The reaction mixture was stirred at 25° C. for 1 h and at 100° C. for 3 h. The reaction mixture was diluted with water (60 mL) and extracted with ethyl acetate (3×50 mL). The combined organics were dried (Na₂SO₄), concentrated under reduced pressure and the residue purified by silica gel chromatography (0-25% EtOAc/PE) to afford the title compound as an off-white solid (730 mg, 94%). LCMS: m/z=313 [M+H]⁺.

### Step 5. Synthesis of tert-butyl(1-(5-((diphenylmethylene)amino)pyridin-3-yl)cyclopropyl)carbamate

A mixture of tert-butyl(1-(5-bromopyridin-3-yl)cyclopropyl)carbamate (Step 4, 365 mg, 2.01 mmol), diphenylmethanimine (600 mg, 1.92 mmol), caesium carbonate (1.87 g, 5.75 mmol), XantPhos (222 mg, 0.38 mmol) and Pd₂ (dba)₃ (175 mg, 0.19 mmol) in dioxane (15 mL) was degassed and purged with N₂ (3×) and stirred at 100° C. for 3 h under N₂. The reaction mixture was concentrated under reduced pressure and the residue purified by silica gel chromatography (0-25% EtOAc/PE) to afford the title compound as a yellow oil (590 mg, 74%). LCMS: m/z=414 [M+H]⁺.

### Step 6. Synthesis of tert-butyl(1-(5-aminopyridin-3-yl)cyclopropyl)carbamate

To a solution of tert-butyl(1-(5-((diphenylmethylene)amino)pyridin-3-yl)cyclopropyl)carbamate (Step 5, 590 mg, 1.43 mmol) in MeOH (20 mL) was added hydroxylamine hydrochloride (3.80 g, 54.7 mmol) and sodium acetate (5.80 g, 70.7 mmol) and the reaction mixture stirred at 25° C. for 2 h. The reaction mixture was added to saturated aqueous sodium bicarbonate (100 mL) concentrated under reduced pressure to remove MeOH and the residue extracted with EtOAc (3×50 mL). The combined organics were dried (Na₂SO₄) and concentrated under reduced pressure. The residue was purified by silica gel chromatography (0-100% EtOAc/PE) to afford the title compound as an off-white solid (330 mg, 93%). LCMS: m/z=198 [M+H].

### Intermediate A41. 3-((ethylamino)methyl) aniline

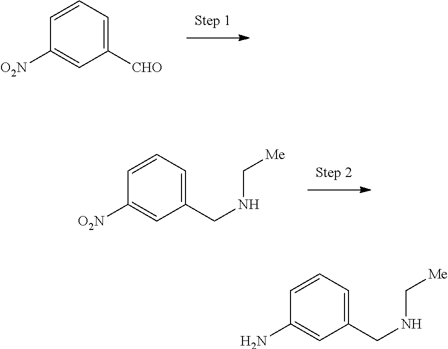 [SMILES: O=Cc1cccc([N+](=O)[O-])c1 ; CCNCc1cccc([N+](=O)[O-])c1 ; CCNCc1cccc(N)c1]

### Step 1. Synthesis of N-(3-nitrobenzyl)ethanamine

To a solution of 3-nitrobenzaldehyde (3 g, 19.85 mmol) in EtOH (40 mL) was added dropwise ethanamine (984 mg, 21.84 mmol) and the mixture stirred 25° C. for 5 h under N₂. NaBH₄ (1.55 g, 41 mmol) was added at 0° C. and the resulting mixture stirred at 25° C. for 2 h under N₂. The reaction mixture was quenched by addition saturated aqueous NH₄Cl solution (30 mL) at 0° C. and extracted with DCM (3×30 mL). The combined organics were dried (Na₂SO₄) and concentrated under reduced pressure to give the title compound as a brown oil (2.4 g, 67%). LCMS m/z=181 [M+H]⁺.

### Step 2. Synthesis of 3-((ethylamino)methyl) aniline

To a solution of N-(3-nitrobenzyl) ethanamine (2.4 g, 13.32 mmol) in MeOH (30 mL) was added Pd/C (3 g, 10% purity) and the mixture stirred at 25° C. for 2 h under H₂ (15 psi). The reaction mixture was filtered and the filtrate was concentrated under reduced pressure and the residue purified by prep-HPLC-18 (5-35% MeCN) to give the title compound as a brown oil (1.08 g, 54%). LCMS m/z=151 [M+H]⁺.

### Intermediate A42. 5,6-dihydrospiro[cyclopenta[c]pyridine-7,2′-pyrrolidin]-4-amine

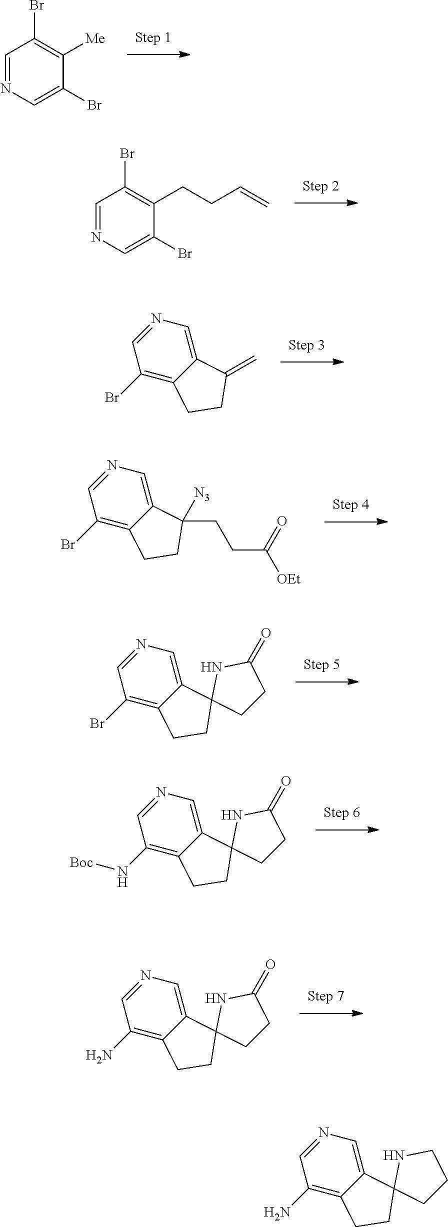

### Step 1. Synthesis of 3,5-dibromo-4-(but-3-en-1-yl)pyridine

To a solution of 3,5-dibromo-4-methyl-pyridine (25 g, 99.63 mmol) in THF (200 mL) was added LDA (2M, 64.76 mL) at −78° C. under N₂ and after 45 mins, 3-bromoprop-1-ene (18.08 g, 149 mmol) in THF (30 mL) was added and the mixture was stirred at −78° C. for 2 h under N₂. The reaction mixture was quenched by addition saturated aqueous NH₄Cl solution (200 mL) at 0° C. and extracted with EtOAc (3×100 mL). The combined organics were dried (Na₂SO₄) and concentrated under reduced pressure and the residue purified by MPLC (SiO₂, 1-10% EtOAc/PE) to give the title compound as a brown oil (27.8 g, 96%). LCMS m/z=292 [M+H]⁺.

### Step 2. Synthesis of 4-bromo-7-methylene-6,7-dihydro-5H-cyclopenta[c]pyridine

To a solution of 3,5-dibromo-4-(but-3-en-1-yl)pyridine (Step 1, 26.8 g, 92.1 mmol) in MeCN (200 mL) was added TEA (18.64 g, 184 mmol), Pd(OAc)₂ (1.03 g, 4.61 mmol) and PPh₃ (2.42 g, 9.21 mmol) and the mixture stirred at 80° C. for 24 h under N₂. The reaction mixture was concentrated under reduced pressure and the residue purified by MPLC (SiO₂, 1-16% EtOAc/PE) to give the title compound as a white solid (12.5 g, 65%). LCMS m/z=210 [M+H]⁺.

### Step 3. Synthesis of ethyl 3-(7-azido-4-bromo-6,7-dihydro-5H-cyclopenta[c]pyridin-7-yl)propanoate

To a solution of 4-bromo-7-methylene-6,7-dihydro-5H-cyclopenta[c]pyridine (3.1 g, 14.76 mmol) and ethyl 2-diazoacetate (3.37 g, 29.51 mmol) in i-PrOH (50 mL) was added diacetoxyiron (257 mg, 1.48 mmol), TEA (2.99 g, 29.51 mmol), TMSN₃ (3.40 g, 29.51 mmol) and TBHP (5.5 M, 8.05 mL) and the mixture stirred at 50° C. for 2 h under N₂. The reaction mixture was quenched by addition saturated aqueous Na₂SO₃ solution (50 mL) at 0° C. and then extracted with EtOAc (3×80 mL). The combined organics were dried (Na₂SO₄) and concentrated under reduced pressure to give the title compound as a brown oil (1.98 g, 39%). LCMS m/z=339 [M+H]⁺.

### Step 4. Synthesis of 4-bromo-5,6-dihydrospiro[cyclopenta[c]pyridine-7,2′-pyrrolidin]-5′-one

To a solution of ethyl 3-(7-azido-4-bromo-6,7-dihydro-5H-cyclopenta[c]pyridin-7-yl)propanoate (Step 3, 1.52 g, 4.48 mmol) in THF (20 mL), i-PrOH (4 mL), AcOH (4 mL) was added Zn (2.91 g, 44.5 mmol) and the mixture stirred at 40° C. for 2 h. The reaction mixture was filtered and the filtrate concentrated under reduced pressure to give a residue which was diluted with H₂O (70 mL), extracted with EtOAc (40 mL), dried (Na₂SO₄) and concentrated under reduced pressure. The residue was purified by MPLC (SiO₂, 1-100% EtOAc/PE) to give the title compound as a white solid (573 mg, 48%). LCMS m/z=267 [M+H]⁺.

### Step 5. Synthesis of tert-butyl(5′-oxo-5,6-dihydrospiro[cyclopenta[c]pyridine-7,2′-pyrrolidin]-4-yl)carbamate

A mixture of 4-bromo-5,6-dihydrospiro[cyclopenta[c]pyridine-7,2′-pyrrolidin]-5′-one (Step 4, 553 mg, 2.07 mmol), tert-butyl carbamate (970 mg, 8.28 mmol), Pd₂ (dba)₃ (190 mg, 0.207 mmol), XPhos (98.7 mg, 0.207 mmol) and Cs₂CO₃ (1.01 g, 3.11 mmol) in dioxane (10 mL) and the mixture stirred at 100° C. for 4 h under N₂. The reaction mixture was filtered and the filtrate concentrated under reduced pressure and the residue purified by MPLC (SiO₂, 1-100% EtOAc/PE) to give the title compound as a brown solid (360 mg, 57%). LCMS m/z=304 [M+H]⁺.

### Step 6. Synthesis of 4-amino-5,6-dihydrospiro[cyclopenta[c]pyridine-7,2′-pyrrolidin]-5′-one

A mixture of tert-butyl(5′-oxo-5,6-dihydrospiro[cyclopenta[c]pyridine-7,2′-pyrrolidin]-4-yl)carbamate (116 mg, 0.382 mmol) in HCl/EtOAc (2 mL) was stirred at 25° C. for 1 h. The reaction mixture was concentrated under reduced pressure to give the title compound as a white solid (112 mg). LCMS m/z=204 [M+H]⁺.

### Step 7. Synthesis of 5,6-dihydrospiro[cyclopenta[c]pyridine-7,2′-pyrrolidin]-4-amine

To a solution of 4-amino-5,6-dihydrospiro[cyclopenta[c]pyridine-7,2′-pyrrolidin]-5′-one (Step 6, 137 mg, 0.674 mmol) in THF (2 mL) was added borane dimethylsulphide complex (10 M, 0.674 mL) at 0° C. and the mixture stirred at 60° C. for 2 h under N₂. The reaction mixture was quenched by addition 1M HCl (2 mL) at 0° C. and then concentrated under reduced pressure. The residue was purified by prep-HPLC-16 (1-30% MeCN) to give the title compound as a yellow oil (45 mg, 35%). LCMS m/z=190 [M+H]⁺.

### Intermediate A43. (R)-7-methyl-6,7-dihydro-5H-cyclopenta[c]pyridine-4,7-diamine or (S)-7-methyl-6,7-dihydro-5H-cyclopenta[c]pyridine-4,7-diamine

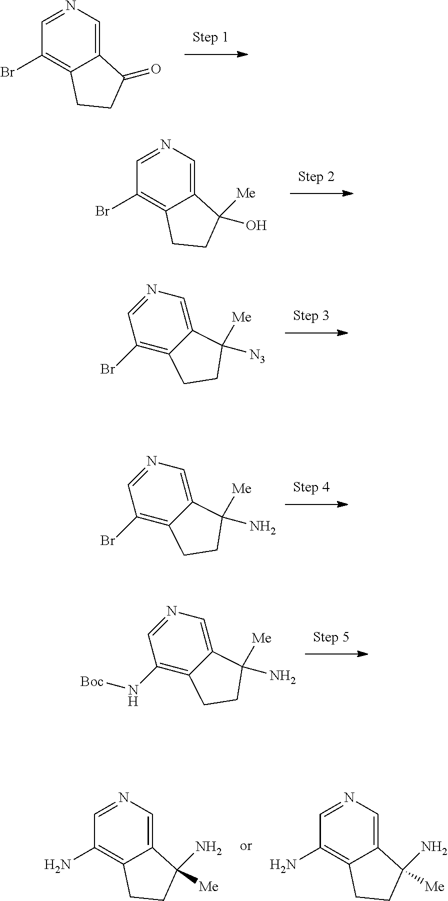

### Step 1. Synthesis of 4-bromo-7-methyl-6,7-dihydro-5H-cyclopenta[c]pyridin-7-ol

To a mixture of 4-bromo-5,6-dihydro-7H-cyclopenta[c]pyridin-7-one (100 mg, 0.472 mmol) in THF (2.5 mL) was added MeMgBr (3M, 471 μL) in THF at −78° C. and the mixture was stirred at 25° C. for 18 h under N₂ and then stirred at 80° C. for 1 h under N₂. The reaction mixture was quenched by addition of saturated aqueous NH₄Cl solution (10 mL) at 0° C. and then extracted with EtOAc (3×15 mL). The combined organics were dried (Na₂SO₄) and concentrated under reduced pressure to give a residue that was purified by column chromatography on silica gel (0-50% EtOAc/PE) to give the title compound as a white solid (400 mg, 53%). LCMS m/z=228 [M+H]⁺.

### Step 2. Synthesis of 7-azido-4-bromo-7-methyl-6,7-dihydro-5H-cyclopenta[c]pyridine

To a solution of 4-bromo-7-methyl-6,7-dihydro-5H-cyclopenta[c]pyridin-7-ol (Step 1, 120 mg, 0.526 mmol) in DCM (2 mL) was added TMSN₃ (182 mg, 1.58 mmol) under N₂. Indium tribromide (373 mg, 1.05 mmol) was added and the mixture stirred at 0° C. and at 25° C. for 12 h. The reaction mixture was filtered and the filtrate concentrated under reduced pressure. The residue was purified by column chromatography on silica gel (0-50% EtOAc/PE) to give the title compound as a yellow oil (250 mg, 62%). LCMS m/z=253 [M+H]⁺.

Step 3. Synthesis of 4-bromo-7-methyl-6,7-dihydro-5H-cyclopenta[c]pyridin-7-amine.

Zn (310 mg, 4.74 mmol) was added to a solution of 7-azido-4-bromo-7-methyl-6,7-dihydro-5H-cyclopenta[c]pyridine (Part 2, 200 mg, 0.79 mmol) in THF (4 mL), IPA (1 mL) and AcOH (1 mL) under N₂ and the mixture stirred at 20° C. for 8 h. The reaction mixture was filtered and the filtrate concentrated under reduced pressure and the residue purified by prep-HPLC-15 (5-45% MeCN) to give the title compound as a yellow solid (150 mg, 83%). LCMS m/z=227 [M+H]⁺.

### Step 4. Synthesis of tert-butyl(7-amino-7-methyl-6,7-dihydro-5H-cyclopenta[c]pyridin-4-yl)carbamate

To the mixture of 4-bromo-7-methyl-6,7-dihydro-5H-cyclopenta[c]pyridin-7-amine (Step 3, 100 mg, 0.44 mmol) and tert-butyl carbamate (103 mg, 0.88 mmol) in dioxane (4 mL) was added Pd₂ (dba)₃ (40.3 mg, 0.044 mmol) and XPhos (21 mg, 0.044 mmol) and Cs₂CO₃ (287 mg, 0.88 mmol) and the mixture stirred at 100° C. for 2 h under N₂. The reaction mixture was filtered and the filtrate was concentrated under reduced pressure to give the title compound as a yellow oil (100 mg, 86%). LCMS m/z=264 [M+H]⁺.

### Step 5. Synthesis of (R)-7-methyl-6,7-dihydro-5H-cyclopenta[c]pyridine-4,7-diamine and (S)-7-methyl-6,7-dihydro-5H-cyclopenta[c]pyridine-4,7-diamine

A mixture of tert-butyl(7-amino-7-methyl-6,7-dihydro-5H-cyclopenta[c]pyridin-4-yl)carbamate (Step 4) in EtOAc (1 mL) and HCl/EtOAc (1 mL) was stirred at 25° C. for 1 h. The reaction mixture was concentrated under N₂ and the residue purified by prep-HPLC (Phenomenex Luna C18 200×40 mm, 10 μm; 1-15% MeCN/H₂O(HCO₂H)) followed by prep-SFC (Diacel Chiralpak AD, 250×30 mm, 10 μm; 25% MeOH (+0.1% NH₄OH) in CO₂) to afford:

Peak 2, Intermediate A43. (R)-7-methyl-6,7-dihydro-5H-cyclopenta[c]pyridine-4,7-diamine or (S)-7-methyl-6,7-dihydro-5H-cyclopenta[c]pyridine-4,7-diamine (yellow oil, 30 mg, 50%); LCMS m/z=164 [M+H]⁺.

### Intermediate A44 and A45. tert-butyl(S)-(1-(5-aminopyridin-3-yl)-3,3-difluoropropyl)carbamate and tert-butyl (R)-(1-(5-aminopyridin-3-yl)-3,3-difluoropropyl)carbamate

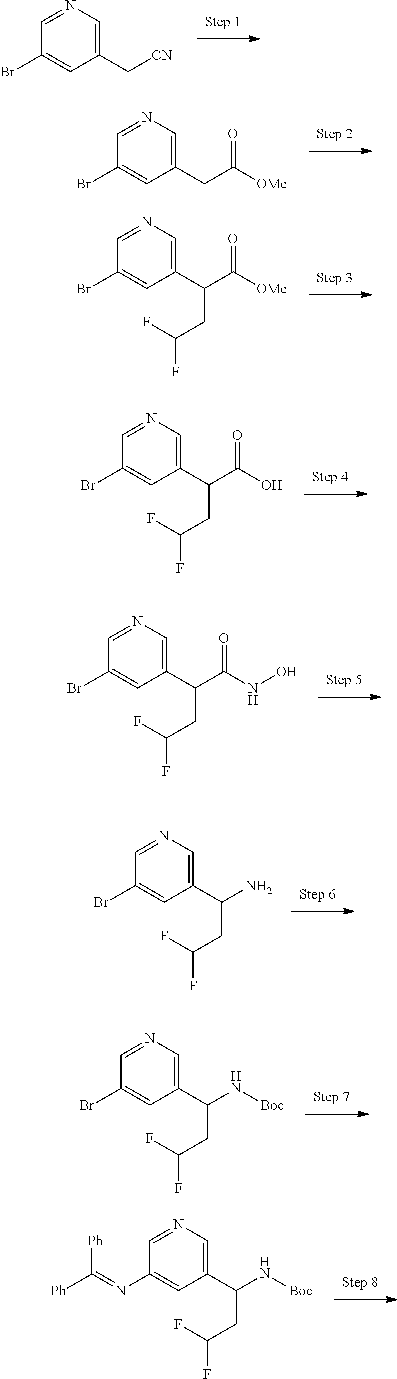

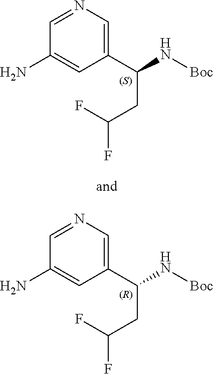

### Step 1. Synthesis of methyl 2-(5-bromopyridin-3-yl)acetate

A mixture of 2-(5-bromo-3-pyridyl) acetonitrile (10 g, 50.75 mmol) in HCl/MeOH (30 mL) was stirred at 25° C. for 12 h. The mixture was treated with saturated aqueous Na₂CO₃ until pH=8, diluted with H₂O (50 mL) and extracted with EtOAc (3×50 mL). The combined organics were washed with brine (3×50 ml), dried (Na₂SO₄) and concentrated under reduced pressure to give the title compound as a yellow oil (11.63 g, 99%). LCMS m/z=230 [M+H]⁺.

### Step 2. Synthesis of methyl 2-(5-bromopyridin-3-yl)-4,4-difluorobutanoate

To a solution of methyl 2-(5-bromopyridin-3-yl)acetate (Part 1, 11.0 g, 47.8 mmol) in THF (250 mL) was added LDA (2M, 35.86 mL) at −78° C. over 30 min. To this was added a solution of 2,2-difluoroethyl trifluoromethanesulfonate (15.36 g, 71.72 mmol) in THF (20 mL) and the mixture stirred at 25° C. for 1 h under N₂. The reaction mixture was quenched by addition H₂O (50 mL) at 0° C., and extracted with EtOAc (3×50 mL). The combined organics were dried (Na₂SO₄), concentrated under reduced pressure and the residue purified by column chromatography on silica gel (0-25% EtOAc/PE) to give the title compound as a yellow oil (10.6 g, 75%). LCMS m/z=294 [M+H]⁺

### Step 3. Synthesis of 2-(5-bromopyridin-3-yl)-4,4-difluorobutanoic acid

To a solution of methyl 2-(5-bromopyridin-3-yl)-4,4-difluorobutanoate (Step 2, 10.60 g, 36.04 mmol) in THF (20 mL) and H₂O (5 mL) was added LiOH (2.59 g, 108 mmol) and the mixture stirred at 25° C. for 1 h. The mixture was acidified to pH 2 with 1M HCl and extracted with EtOAc (3×50 mL). The combined organics were washed with brine, dried (Na₂SO₄) and concentrated under reduced pressure to give the title compound as a yellow solid (8.08 g, 80%). LCMS m/z=280 [M+H]⁺

### Step 4. Synthesis of 2-(5-bromopyridin-3-yl)-4,4-difluoro-N-hydroxybutanamide

To a solution of 2-(5-bromopyridin-3-yl)-4,4-difluorobutanoic acid (Step 3, 8.08 g, 28.9 mmol) and hydroxylamine hydrochloride (2.41 g, 34.62 mmol) in DMF (20 mL) was added DIPEA (11.2 g, 86.6 mmol) and BOP (16.59 g, 37.5 mmol) and the mixture stirred at 25° C. for 1 h. The reaction mixture was concentrated under reduced pressure and residue was diluted with H₂O (100 mL) and extracted with EtOAc (3×100 mL), dried (Na₂SO₄) and concentrated under reduced pressure. The residue was purified by column chromatography on silica gel (0-100% EtOAc/PE) to give the title compound as a yellow oil (7.60 g, crude). LCMS m/z=295 [M+H]⁺

### Step 5. Synthesis of 1-(5-bromopyridin-3-yl)-3,3-difluoropropan-1-amine

K₂CO₃ (7.10 g, 51.38 mmol) was added to a solution of 2-(5-bromopyridin-3-yl)-4,4-difluoro-N-hydroxybutanamide (Part 4, 7.58 g, 25.69 mmol) in DMSO (50 mL) and the mixture stirred at 80° C. for 1 h. The reaction mixture was concentrated under reduced pressure and the residue diluted with H₂O (100 mL) and extracted with EtOAc (3×100 mL), dried (Na₂SO₄) and concentrated under reduced pressure to give the title compound as a yellow oil (4.00 g, crude). LCMS m/z=251 [M+H]⁺

### Step 6. Synthesis of tert-butyl(1-(5-bromopyridin-3-yl)-3,3-difluoropropyl)carbamate

To a solution of-(5-bromopyridin-3-yl)-3,3-difluoropropan-1-amine (Step 5, 4 g, 15.9 mmol) in dioxane (20 mL) and H₂O (4 mL) was added NaHCO₃ (2.68 g, 31.9 mmol) and Boc₂O (3.48 g, 15.9 mmol) and the mixture stirred at 25° C. for 1 h. The reaction mixture was concentrated under reduced pressure, diluted with H₂O (50 mL) and extracted with EtOAc (3×50 mL). The combined organics were dried (Na₂SO₄) and concentrated under reduced pressure and the residue was purified by column chromatography on silica gel (0-25% EtOAc/PE) to give the title compound as a white solid (3.70 g, 66%). LCMS m/z=351 [M+H]⁺.

### Step 7. Synthesis of tert-butyl(1-(5-((diphenylmethylene)amino)pyridin-3-yl)-3,3-difluoropropyl)carbamate

To a solution of tert-butyl(1-(5-bromopyridin-3-yl)-3,3-difluoropropyl)carbamate (Step 6, 3.70 g, 10.54 mmol) and diphenylmethanimine (2.10 g, 11.59 mmol, 1.94 mL, 1.10 eq) in dioxane (30 mL) was added Cs₂CO₃ (10.3 g, 31.61 mmol), Xantphos (1.22 g, 2.11 mmol) and Pd₂ (dba)₃ (965 mg, 1.05 mmol) and the mixture stirred at 100° C. for 3 h under N₂. The reaction mixture was concentrated under reduced pressure and the residue diluted with H₂O (100 mL) and extracted with EtOAc (3×100 mL). The combined organics were dried (Na₂SO₄) and concentrated under reduced pressure and the residue purified by column chromatography on silica gel (0-25% EtOAc/PE) to give the title compound as a yellow oil (3.45 g, 72%). LCMS m/z=452 [M+H]⁺.

### Step 8. Synthesis of tert-butyl(S)-(1-(5-aminopyridin-3-yl)-3,3-difluoropropyl)carbamate and tert-butyl (R)-(1-(5-aminopyridin-3-yl)-3,3-difluoropropyl)carbamate

To a solution of tert-butyl(1-(5-((diphenylmethylene)amino)pyridin-3-yl)-3,3-difluoropropyl)carbamate (Step 7, 3.45 g, 7.64 mmol) in MeOH (30 mL) was added NH₂OH·HCl (1.06 g, 15.28 mmol) and NaOAc (1.25 g, 15.28 mmol) and the mixture stirred at 25° C. for 2 h. The reaction mixture was concentrated under reduced pressure. The residue was diluted with H₂O (100 mL) and extracted with EtOAc (3×100 mL), dried (Na₂SO₄) and concentrated under reduced pressure. The residue was purified by column chromatography on silica gel (0-100% EtOAc/PE) followed by chiral SFC (DAICEL CHIRALPAK AD, 250×50 mm, 10 μm); 25% EtOH (+0.1% NH₄OH) in CO₂) to afford:

Peak 1, Intermediate A44. tert-butyl(S)-(1-(5-aminopyridin-3-yl)-3,3-difluoropropyl)carbamate or tert-butyl (R)-(1-(5-aminopyridin-3-yl)-3,3-difluoropropyl)carbamate (yellow solid, 800 mg, 36%); LCMS m/z=288 [M+H]⁺

Peak 2, Intermediate A45. tert-butyl(S)-(1-(5-aminopyridin-3-yl)-3,3-difluoropropyl)carbamate or tert-butyl (R)-(1-(5-aminopyridin-3-yl)-3,3-difluoropropyl)carbamate (yellow solid, 880 mg, 40%); LCMS m/z=288 [M+H]⁺.

### Intermediate A46. (1S,3aR,6aS)-2-(tert-butoxycarbonyl)octahydrocyclopenta[c]pyrrole-1-carboxylic acid

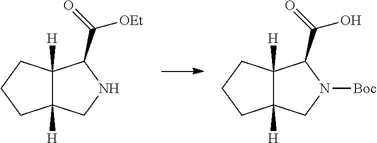 [SMILES: CCOC(=O)[C@H]1NC[C@@H]2CCC[C@@H]21 ; CC(C)(C)OC(=O)N1C[C@@H]2CCC[C@@H]2[C@H]1C(=O)O]

A mixture of ethyl(1S,3aR,6aS)-octahydrocyclopenta[c]pyrrole-1-carboxylate hydrochloride (4.60 g, 20.94 mmol) and LiOH·H₂O (5.27 g, 126 mmol) in MeOH (20 mL), THF (20 mL) and H₂O (20 mL) was stirred at 25° C. for 12 h. (Boc)₂₀ (9.14 g, 41.87 mmol) was added and the mixture stirred at 25° C. for 8 h. The reaction mixture was filtered and the filtrate concentrated under reduced pressure and the residue was treated with EtOAc (30 mL) and stirred at 20° C. for 0.5 h. The mixture was filtered and the filtrate concentrated under reduced pressure to give the title compound as a white solid (4.20 g, 78%). LCMS m/z=256 [M+H]⁺.

### Intermediate A47. 5-((3aS,6aR)-octahydrocyclopenta[c]pyrrol-1-yl)pyridin-3-amine

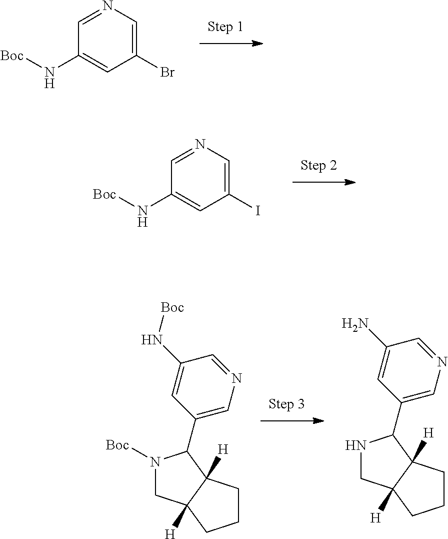 [SMILES: CC(C)(C)OC(=O)Nc1cncc(Br)c1 ; CC(C)(C)OC(=O)Nc1cncc(I)c1 ; CC(C)(C)OC(=O)Nc1cncc(C2[C@@H]3CCC[C@@H]3CN2C(=O)OC(C)(C)C)c1 ; Nc1cncc(C2NC[C@H]3CCC[C@@H]23)c1]

### Step 1. Synthesis of tert-butyl(5-iodopyridin-3-yl)carbamate

A mixture of tert-butyl N-(5-bromopyridin-3-yl)carbamate (5 g, 18.31 mmol), CuI (349 mg, 1.83 mmol), NaI (5.49 g, 36.6 mmol) and N′,N′-dimethylethane-1,2-diamine (2.42 g, 27.5 mmol) in dioxane (30 mL) was stirred at 110° C. for 24 h under N₂. The reaction mixture was filtered and the filtrate concentrated under reduced pressure and the residue purified by column chromatography (SiO₂, 0-50% EtOAc/PE/) to give the title compound as a white solid (4 g, 68%). LCMS m/z=321 [M+H]⁺.

### Step 2. Synthesis of tert-butyl(3aS,6aR)-1-(5-((tert-butoxycarbonyl)amino)pyridin-3-yl)hexahydrocyclopenta[c]pyrrole-2 (1H)-carboxylate

To a solution of tert-butyl(5-iodopyridin-3-yl)carbamate (Step 1, 163 mg, 0.51 mmol) and (1S,3aR,6aS)-2-(tert-butoxycarbonyl)octahydrocyclopenta[c]pyrrole-1-carboxylic acid (Intermediate 46, 100 mg, 0.392 mmol) in DMF (3 mL) was added (Ir[dF(CF₃)ppy]2(dtbpy))PF₆ (4.39 mg, 3.92 μmol), dichloronickel; 1,2-dimethoxyethane (8.61 mg, 39.2 μmol), Cs₂CO₃ (383 mg, 1.18 mmol) and 4-tert-butyl-2-(4-tert-butyl-2-pyridyl)pyridine (15.8 mg, 59 μmol) and the mixture stirred at 25° C. for 12 h. The reaction mixture was filtered and the filtrate concentrated under reduced pressure and the residue purified by prep-HPLC-11 (40-75% MeCN) to give the title compound as a yellow oil (150 mg, 47%). LCMS m/z=402 [M+H]⁺.

### Step 3. Synthesis of 5-((3aS,6aR)-octahydrocyclopenta[c]pyrrol-1-yl)pyridin-3-amine

tert-butyl(3aS,6aR)-1-(5-((tert-butoxycarbonyl)amino)pyridin-3-yl)hexahydrocyclopenta[c]pyrrole-2 (1H)-carboxylate (Step 2, 150 mg, 0.372 mmol) in HCl/EtOAc (1 mL) and EtOAc (0.5 mL) was stirred at 25° C. for 6 h. The reaction mixture concentrated under reduced pressure and the residue was diluted with EtOAc (10 mL) and treated with NH₄OH (0.5 mL) and concentrated under reduced pressure. The residue was purified by prep-HPLC-15 (1-30% MeCN) to give the title compound as a white solid (50 mg, 66%). LCMS m/z=204 [M+H]⁺.

### Intermediate A48. (R)-2-(1-(5-aminopyridin-3-yl)-2,2-difluoroethyl) isoindoline-1,3-dione or (S)-2-(1-(5-aminopyridin-3-yl)-2,2-difluoroethyl) isoindoline-1,3-dione

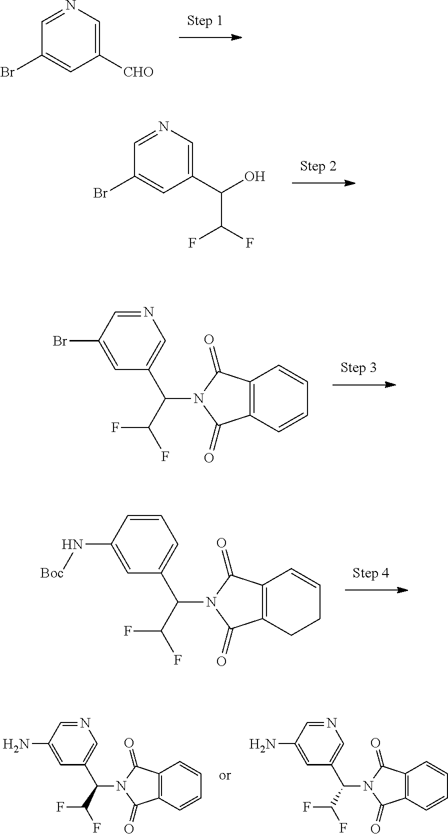 [SMILES: O=Cc1cncc(Br)c1 ; OC(c1cncc(Br)c1)C(F)F ; O=C1c2ccccc2C(=O)N1C(c1cncc(Br)c1)C(F)F ; CC(C)(C)OC(=O)Nc1cccc(C(C(F)F)N2C(=O)C3=C(CCC=C3)C2=O)c1 ; Nc1cncc([C@H](C(F)F)N2C(=O)c3ccccc3C2=O)c1 ; Nc1cncc([C@@H](C(F)F)N2C(=O)c3ccccc3C2=O)c1]

### Step 1. Synthesis of 1-(5-bromopyridin-3-yl)-2,2-difluoroethan-1-ol

To a solution of 5-bromopyridine-3-carbaldehyde (5 g, 26.88 mmol) and difluoromethyl(trimethyl) silane (5.01 g, 40.32 mmol) in DMF (50 mL) was added CsF (817 mg, 5.38 mmol) and the mixture stirred at 25° C. for 12 h under N₂. TBAF (1M, 40.32 mL) was added and the mixture stirred at 25° C. for 1 h under N₂. The reaction mixture was concentrated under reduced pressure and the residue diluted with H₂O (70 mL) and extracted with EtOAc (3×50 mL). The combined organics were dried (Na₂SO₄) and concentrated under reduced pressure. The residue was purified by MPLC (SiO₂, 50% PE/EtOAc) to give the title compound as a yellow oil (1.3 g, 20% yield). LCMS m/z=238 [M+H]⁺.

### Step 2. Synthesis of 2-(1-(5-bromopyridin-3-yl)-2,2-difluoroethyl) isoindoline-1,3-dione

To a solution of 1-(5-bromopyridin-3-yl)-2,2-difluoroethan-1-ol (Step 1, 1.2 g, 5.04 mmol) and isoindoline-1,3-dione (816 mg, 5.55 mmol) in THF (30 mL) was added PPh₃ (1.45 g, 5.55 mmol) and DEAD (1.05 g, 6.05 mmol) and the mixture stirred at 25° C. for 2 h under N₂.

The reaction mixture was concentrated under reduced and the residue purified by MPLC (SiO₂, 50% PE/EtOAc) to give the title compound as a yellow oil (950 mg, 51%). LCMS m/z=367 [M+H]⁺.

### Step 3. Synthesis of tert-butyl(5-(1-(1,3-dioxoisoindolin-2-yl)-2,2-difluoroethyl)pyridin-3-yl)carbamate

To the mixture of 2-(1-(5-bromopyridin-3-yl)-2,2-difluoroethyl) isoindoline-1,3-dione (Step 2, 900 mg, 2.45 mmol) and tert-butyl carbamate (431 mg, 3.68 mmol) in dioxane (15 mL) was added XPhos (117 mg, 0.245 mmol), Cs₂CO₃ (1.20 g, 3.68 mmol) and Pd₂ (dba)₃ (224 mg, 0.245 mmol) and the mixture stirred at 100° C. for 2 h under N₂. The mixture was filtered and the filtrate was evaporated to dryness in vacuo. The residue was purified by MPLC (SiO₂, 0-33% EtOAc/PE) to give the title compound as a yellow solid (500 mg, 50%). LCMS m/z=404 [M+H]⁺.

### Step 4. Synthesis of (R)-2-(1-(5-aminopyridin-3-yl)-2,2-difluoroethyl) isoindoline-1,3-dione or (S)-2-(1-(5-aminopyridin-3-yl)-2,2-difluoroethyl) isoindoline-1,3-dione

tert-Butyl(5-(1-(1,3-dioxoisoindolin-2-yl)-2,2-difluoroethyl)pyridin-3-yl)carbamate (Step 3, 470 mg, 1.17 mmol) was dissolved into HCl/EtOAc (5 mL) and the mixture stirred at 25° C. for 1 h. The reaction mixture was concentrated under reduced pressure and the residue was dissolved in EtOAc and the pH adjusted with NH₄OH to pH=9-10. The reaction mixture was extracted with EtOAc (3×30 mL) and the combined organics dried and concentrated under reduced pressure. The residue was separated by SFC (DAICEL CHIRALCEL OJ, 250×30 mm, 10 μm); 26% MeOH (0.1% NH₄OH) in CO₂) to afford:

Peak 1, Intermediate A48. (R)-2-(1-(5-aminopyridin-3-yl)-2,2-difluoroethyl) isoindoline-1,3-dione or (S)-2-(1-(5-aminopyridin-3-yl)-2,2-difluoroethyl) isoindoline-1,3-dione (yellow oil, 150 mg, 42%); LCMS m/z=304 [M+H]⁺.

### Intermediate A49. 2-(((5-aminopyridin-3-yl)methyl)amino) ethan-1-ol

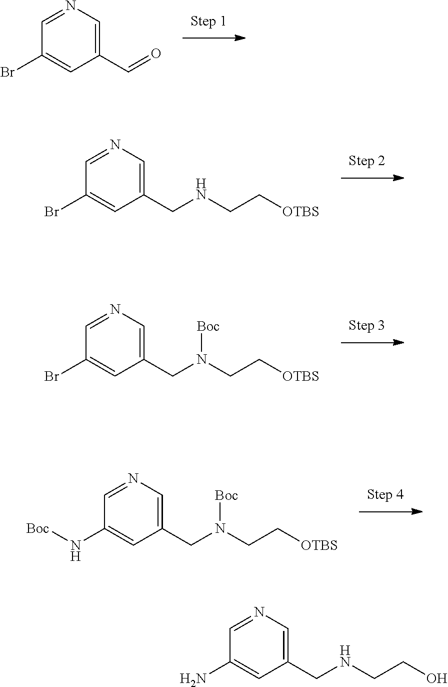 [SMILES: O=Cc1cncc(Br)c1 ; CC(C)(C)[Si](C)(C)OCCNCc1cncc(Br)c1 ; CC(C)(C)OC(=O)N(CCO[Si](C)(C)C(C)(C)C)Cc1cncc(Br)c1 ; CC(C)(C)OC(=O)Nc1cncc(CN(CCO[Si](C)(C)C(C)(C)C)C(=O)OC(C)(C)C)c1 ; Nc1cncc(CNCCO)c1]

### Step 1. Synthesis of N-((5-bromopyridin-3-yl)methyl)-2-((tert-butyldimethylsilyl)oxy) ethan-1-amine

To a solution of 5-bromopyridine-3-carbaldehyde (5 g, 26.9 mmol) in EtOH (20 mL) was added 2-((tert-butyldimethylsilyl)oxy) ethan-1-amine (5.18 g, 29.6 mmol) at 25° C. and the mixture stirred at 25° C. for 12 h under N₂. To this was added NaBH₄ (2.03 g, 53.8 mmol) was added at 0° C. and the resulting mixture stirred at 25° C. for 2 h under N₂. The reaction mixture was quenched by addition saturated NH₄Cl (25 mL) at 0° C. and extracted with EtOAc (3×20 mL). The combined organics were dried over (Na₂SO₄) and concentrated under reduced pressure to give the title compound as a yellow solid (8.7 g, 93%). LCMS m/z=347 [M+H].

### Step 2. Synthesis of tert-butyl ((5-bromopyridin-3-yl)methyl) (2-((tert-butyldimethylsilyl)oxy)ethyl)carbamate

A mixture of N-((5-bromopyridin-3-yl)methyl)-2-((tert-butyldimethylsilyl)oxy) ethan-1-amine (Step 1, 8.5 g, 24.6 mmol), Na₂CO₃ (5.22 g, 49.2 mmol) in THF (30 mL) and H₂O (10 mL) was added di-tert-butyl dicarbonate (5.37 g, 24.6 mmol) and the mixture stirred at 25° C. for 1 h. The reaction mixture was filtered. The filtrate was diluted with H₂O (30 mL) and extracted with EtOAc (3×30 mL). The combined organics were washed with brine (2×20 mL), dried (Na₂SO₄) and concentrated under reduced pressure. The residue was purified by column chromatography (SiO₂, 0-100% EtOAc/PE) to give the title compound as a yellow solid (9.5 g, 86%). LCMS m/z=447 [M+H]⁺.

### Step 3. Synthesis of tert-butyl ((5-((tert-butoxycarbonyl)amino)pyridin-3-yl)methyl) (2-((tert-butyldimethylsilyl)oxy)ethyl)carbamate

A mixture of tert-butyl ((5-bromopyridin-3-yl)methyl) (2-((tert-butyldimethylsilyl)oxy)ethyl)-carbamate (Step 2, 9.3 g, 20.88 mmol), tert-butyl carbamate (4.89 g, 41.75 mmol), Pd₂ (dba)₃ (1.91 g, 2.09 mmol), XPhos (995.24 mg, 2.09 mmol) and Cs₂CO₃ (10.20 g, 31.32 mmol) in dioxane (80 mL) was degassed and purged with N₂ (3×) and the mixture stirred at 100° C. for 2 h under N₂. The reaction mixture was filtered and the filtrate diluted with H₂O (60 mL) and extracted with EtOAc (3×50 mL). The combined organics were washed with brine (2×30 mL), dried (Na₂SO₄) and concentrated under reduced pressure. The residue was purified by column chromatography (SiO₂, 0-100% EtOAc/PE) to give the title compound as a yellow solid (9.5 g, 94%). LCMS m/z=482 [M+H]⁺.

### Step 4. Synthesis of 2-(((5-aminopyridin-3-yl)methyl)amino) ethan-1-ol

A solution of tert-butyl ((5-((tert-butoxycarbonyl)amino)pyridin-3-yl)methyl) (2-((tert-butyldimethylsilyl)oxy)ethyl)carbamate (Step 3, 1.5 g, 3.11 mmol) in HCl/EtOAc (20 mL) was stirred at 25° C. for 12 h. The reaction mixture was concentrated under N₂ and the residue diluted with EtOAc (30 mL) and the pH adjusted to pH>7 with NH₄OH at 0° C. The mixture was filtered and the filtrate concentrated under reduced pressure and the residue purified by prep-HPLC-11 (1-15% MeCN) to give the title compound as a yellow solid (150 mg, 28%). LCMS m/z=168 [M+H]⁺.

### Intermediate A50. tert-butyl ((5-aminopyridin-3-yl)methyl)carbamate

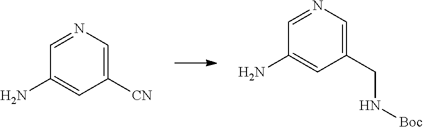 [SMILES: N#Cc1cncc(N)c1 ; CC(C)(C)OC(=O)NCc1cncc(N)c1]

To a solution of 5-aminonicotinonitrile (500 mg, 4.20 mmol) in EtOH (15 mL) was added NiCl₂ (544 mg, 4.20 mmol) and NaBH₄ (635 mg, 16.8 mmol) at 0° C. and the mixture stirred at 25° C. for 8h. To this was added di-tert-butyl dicarbonate (1.37 g, 6.30 mmol) and the mixture stirred for 1 hr. The reaction mixture was quenched by addition saturated aqueous NH₄Cl at 0° C. and then extracted with EtOAc (3×30 mL). The combined organics were dried (Na₂SO₄) and concentrated under reduced pressure. The residue was purified by column chromatography (SiO₂, 0-100% EtOAc/PE) to give the title compound as a yellow solid (550 mg, 58%). LCMS m/z=224 [M+H]⁺.

### Intermediate A51. tert-butyl(1-(5-aminopyridin-3-yl)cyclobutyl)carbamate

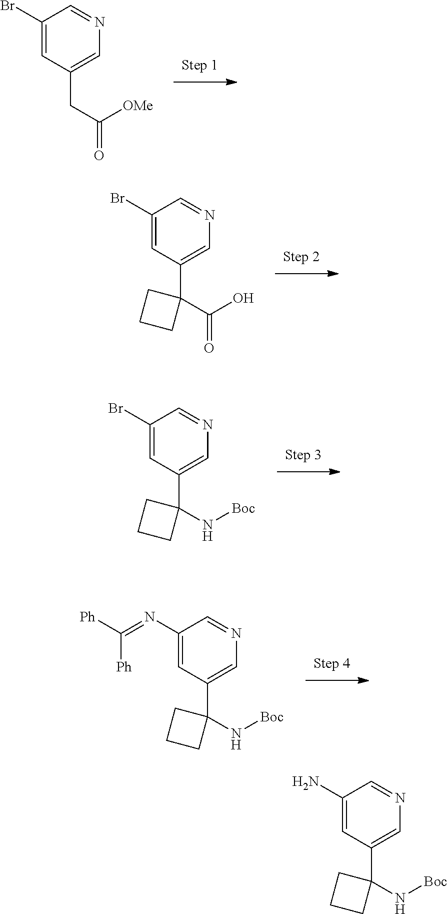 [SMILES: COC(=O)Cc1cncc(Br)c1 ; O=C(O)C1(c2cncc(Br)c2)CCC1 ; CC(C)(C)OC(=O)NC1(c2cncc(Br)c2)CCC1 ; CC(C)(C)OC(=O)NC1(c2cncc(N=C(c3ccccc3)c3ccccc3)c2)CCC1 ; CC(C)(C)OC(=O)NC1(c2cncc(N)c2)CCC1]

### Step 1. Synthesis of 1-(5-bromopyridin-3-yl)cyclobutane-1-carboxylic acid

To a solution of methyl 2-(5-bromopyridin-3-yl)acetate (500 mg, 2.17 mmol) and 1,4,7,10,13,16-hexaoxacyclooctadecane (57.5 mg, 0.217 mmol) in DMF (6 mL) was added NaH (261 mg, 6.52 mmol, 60% purity) at 0° C. and the mixture was stirred at 0° C. for 0.5 h. To this was added 1,3-dibromopropane (483 mg, 2.39 mmol) in DMF (1 mL) and the mixture stirred at 25° C. for 5 h. The reaction mixture was quenched by addition water (30 mL) and extracted with ethyl acetate (3×30 mL) and the combined organics discarded. The aqueous phase was acidified by addition 6 M HCl to pH=2~3 and extracted with EtOAc (3×60 mL). The combined organics were washed with brine (2×30 mL), dried (Na₂SO₄) and concentrated under reduced pressure to give the title compound as a pale yellow oil (540 mg, crude). LCMS m/z=256 [M+H]⁺.

### Step 2. Synthesis of tert-butyl(1-(5-bromopyridin-3-yl)cyclobutyl)carbamate

A mixture of 1-(5-bromopyridin-3-yl)cyclobutane-1-carboxylic acid (Part 1, 500 mg, 1.95 mmol) in t-BuOH (15 mL) was added DPPA (806 mg, 2.93 mmol), TEA (296 mg, 2.93 mmol) and 4 Å molecular sieves (500 mg) and the mixture was stirred at 25° C. for 1 h and then at 100° C. for 1 h. The reaction mixture was diluted with water (30 mL) and extracted with ethyl acetate (3×30 mL). The combined organics were dried over anhydrous sodium sulfate and concentrated under reduced pressure. The residue was purified by column chromatography (SiO₂, 0-50% EtOAc/PE) to give the title compound as a white solid (200 mg, 31%). LCMS m/z=327 [M+H]⁺.

### Step 3. Synthesis of tert-butyl(1-(5-((diphenylmethylene)amino)pyridin-3-yl)cyclobutyl)carbamate

The title compound was prepared as a yellow oil (120 mg, 92%) from tert-butyl(1-(5-bromopyridin-3-yl)cyclobutyl)carbamate (Step 2) using an analogous method to that described for Intermediate A16, Step 3. LCMS m/z=428 [M+H]⁺.

### Step 4. Synthesis of tert-butyl(1-(5-aminopyridin-3-yl)cyclobutyl)carbamate

The title compound was prepared from tert-butyl(1-(5-((diphenylmethylene)amino)pyridin-3-yl)cyclobutyl)carbamate (Step 3) using an analogous method to that described for Intermediate A16, Step 4. LCMS m/z=264 [M+H]⁺.

### Intermediate A52. 5-(2-aminopropan-2-yl)pyridin-3-amine

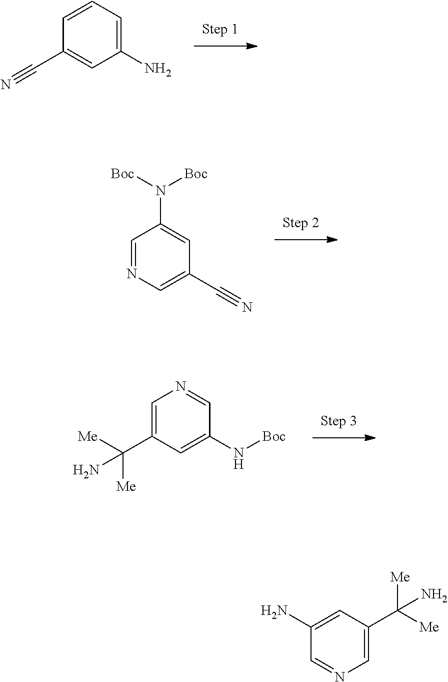 [SMILES: N#Cc1cccc(N)c1 ; CC(C)(C)OC(=O)N(C(=O)OC(C)(C)C)c1cncc(C#N)c1 ; CC(C)(C)OC(=O)Nc1cncc(C(C)(C)N)c1 ; CC(C)(N)c1cncc(N)c1]

### Step 1. Synthesis of tert-butyl (tert-butoxycarbonyl) (5-cyanopyridin-3-yl)carbamate

To the mixture of 5-aminopyridine-3-carbonitrile (5 g, 42 mmol) in DCM (50 mL) was added DMAP (2.56 g, 21 mmol) and TEA (6.37 g, 63 mmol, 8.76 mL) and Boc₂O (13.74 g, 63 mmol) and the mixture stirred at 25° C. for 12 h. The mixture was diluted with H₂O (30 mL) and extracted with EtOAc (3×40 mL). The combined organics were dried (Na₂SO₄) and concentrated under reduced pressure. The residue was purified by column chromatography on silica gel (0-50% EtOAc/PE) to give the title compound as a yellow oil (9.2 g, 68%). LCMS m/z=264 [M+H]⁺.

### Step 2. Synthesis of tert-butyl(5-(2-aminopropan-2-yl)pyridin-3-yl)carbamate

tert-Butyl (tert-butoxycarbonyl) (5-cyanopyridin-3-yl)carbamate (Step 1, 2 g, 6.26 mmol) in THF (25 mL) was stirred with methylmagnesium bromide (3M, 16.70 mL) at 0° C. for 30 min. To this was added Ti(OⁱPr)₄ (1.78 g, 6.26 mmol) was added at 0° C. and the mixture stirred at 70° C. for 2 h under N₂. To the mixture was quenched with H₂O (30 mL) and the mixture filtered and the filter cake was washed with EtOAc (3×40 mL). The aqueous was extracted with EtOAc (3×30 mL) and the combined organics dried (Na₂SO₄) and concentrated. The residue was purified by prep-HPLC-11 (5-45% MeCN) to give the title compound as a yellow oil (500 mg, 31%). LCMS m/z=252 [M+H]⁺.

### Step 3. Synthesis of 5-(2-aminopropan-2-yl)pyridin-3-amine

A solution of tert-butyl(5-(2-aminopropan-2-yl)pyridin-3-yl)carbamate (Step 2, 400 mg, 1.59 mmol) in EtOAc (1 mL) and HCl/EtOAc (5 mL) was stirred at 25° C. for 0.5 h. The reaction mixture was concentrated under N₂ and the pH of the residue adjusted to pH 6-7 with 1M NH₄OH. The residue was purified by prep-HPLC-11 (1-20% MeCN) to give the title compound as a yellow oil (70 mg, 29%). LCMS m/z=152 [M+H].

### Intermediate A53. tert-butyl ((5-aminopyridin-3-yl)methyl)(ethyl-d₅)carbamate

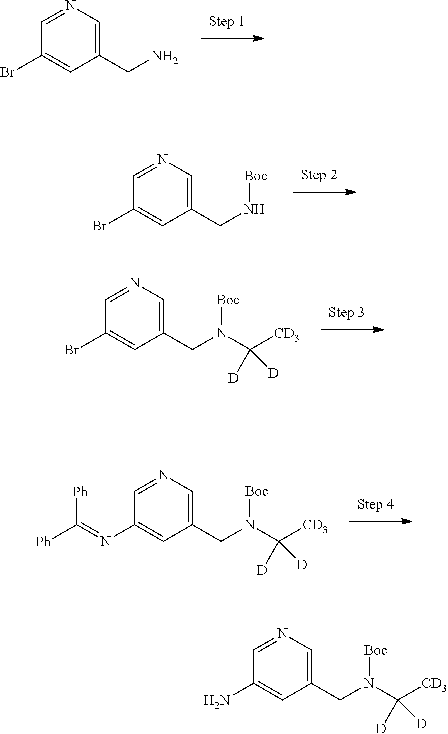 [SMILES: NCc1cncc(Br)c1 ; CC(C)(C)OC(=O)NCc1cncc(Br)c1 ; [2H]C([2H])([2H])C([2H])([2H])N(Cc1cncc(Br)c1)C(=O)OC(C)(C)C ; [2H]C([2H])([2H])C([2H])([2H])N(Cc1cncc(N=C(c2ccccc2)c2ccccc2)c1)C(=O)OC(C)(C)C ; [2H]C([2H])([2H])C([2H])([2H])N(Cc1cncc(N)c1)C(=O)OC(C)(C)C]

### Step 1. Synthesis of tert-butyl ((5-bromopyridin-3-yl)methyl)carbamate

To the mixture of (5-bromo-3-pyridyl) methanamine (4.9 g, 26.20 mmol) in THF (40 mL) and H₂O (4 mL) was added NaHCO₃ (4.40 g, 52.4 mmol) and di-tert-butyl dicarbonate (6.29 g, 28.8 mmol) and the mixture stirred at 20° C. for 0.5 h. The reaction mixture was partitioned between EtOAc (20 mL) and H₂O (15 mL) and the water phase extracted with EtOAc (3×20 mL). The combined organics were dried (Na₂SO₄) and concentrated under reduced pressure. The residue was purified by MPLC (SiO₂, 0-50% EtOAc/PE) to give the title compound as a white solid (6.5 g, 86%). LCMS m/z=289 [M+H]⁺.

### Step 2. Synthesis of tert-butyl ((5-bromopyridin-3-yl)methyl)(ethyl-d₅)carbamate

To a solution of tert-butyl ((5-bromopyridin-3-yl)methyl)carbamate (Step 1, 450 mg, 1.57 mmol) in DMF (3 mL) was added NaH (125 mg, 3.13 mmol, 60% purity) at 0° C. and the mixture stirred at 0° C. for 0.5 h. To this was added a solution of 1-iodoethane-1,1,2,2,2-d₅ (378 mg, 2.35 mmol) in DMF (0.5 mL) and the mixture was stirred at 0° C. for 0.5 h. NaCl (sat aq, 5 mL) was added and the reaction mixture partitioned between EtOAc (10 mL) and H₂O (5 mL). The aqueous phase was extracted with EtOAc (3×10 mL). The combined organics were dried (Na₂SO₄) and concentrated under reduced pressure and the residue purified by MPLC (SiO₂, 0-25% EtOAc/PE) to give the title compound as a white oil (480 mg, 95%). LCMS m/z=322 [M+H]⁺.

### Step 3. Synthesis of tert-butyl ((5-((diphenylmethylene)amino)pyridin-3-yl)methyl)(ethyl-d₅)carbamate

The title compound was prepared as a yellow oil (480 mg, 87%) from tert-butyl ((5-bromopyridin-3-yl)methyl)(ethyl-d₅)carbamate (Step 2) using an analogous method to that described for Intermediate A16 (Step 3). LCMS m/z=421 [M+H]⁺.

### Step 4. Synthesis of tert-butyl ((5-aminopyridin-3-yl)methyl)(ethyl-d₅)carbamate

The title compound was prepared from tert-butyl(1-(5-((diphenylmethylene)amino)pyridin-3-yl)cyclobutyl)carbamate (Step 3) using an analogous method to that described for Intermediate A16. LCMS m/z=257 [M+H]⁺.

### Intermediate A54. (S)-5-(1-((methyl-d₃)amino)ethyl)pyridin-3-amine or (R)-5-(1-((methyl-d₃)amino)ethyl)pyridin-3-amine

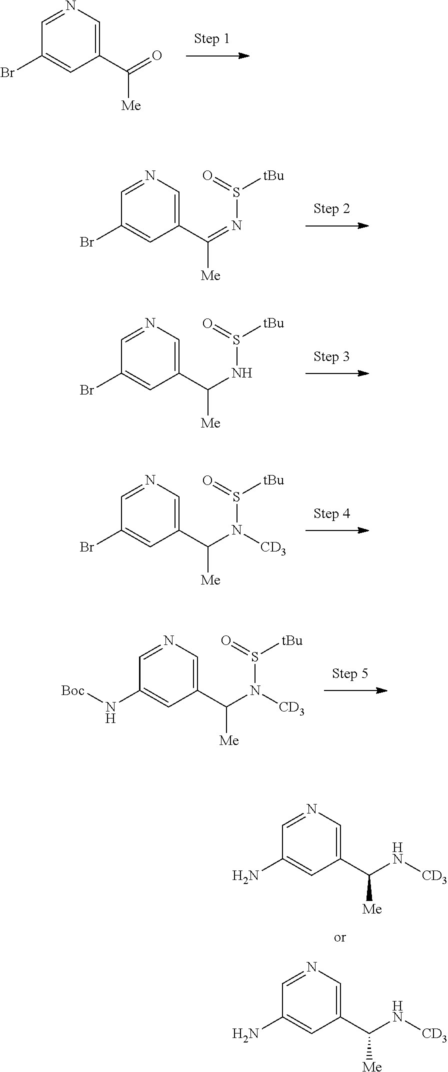

The title compounds were prepared from 1-(5-bromopyridin-3-yl) ethan-1-one using an analogous 5 Step method as described for Intermediate A14 and A15. Chiral-SFC (DAICEL CHIRALPAK IG, 250×30 mm, 10 μm); 30% Heptane/EtOH (+0.1% NH₄OH) in CO₂) afforded:

Peak 1, Intermediate A54: (S)-5-(1-((methyl-d₃)amino)ethyl)pyridin-3-amine or (R)-5-(1-((methyl-d₃)amino)ethyl)pyridin-3-amine (yellow solid, 600 mg, 40%). LCMS m/z=155 [M+H]⁺.

### Intermediate A55. 5-(3-aminotetrahydro-2H-pyran-3-yl)pyridin-3-amine

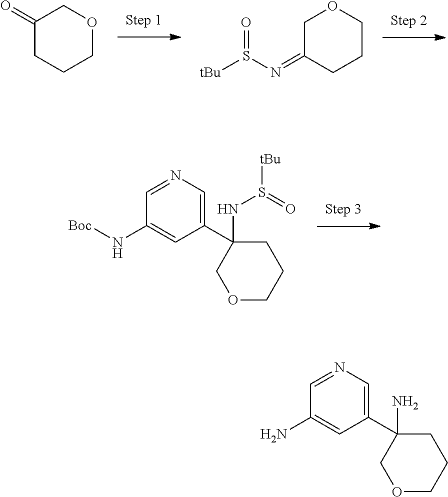 [SMILES: O=C1CCCOC1 ; CC(C)(C)S(=O)/N=C1/CCCOC1 ; CC(C)(C)OC(=O)Nc1cncc(C2(NS(=O)C(C)(C)C)CCCOC2)c1 ; Nc1cncc(C2(N)CCCOC2)c1]

### Step 1. Synthesis of (Z)—N-(dihydro-2H-pyran-3 (4H)-ylidene)-2-methylpropane-2-sulfinamide

Ti(OEt)₄ (29.62 g, 130 mmol) was added to a mixture of tetrahydropyran-3-one (10 g, 100 mmol) and 2-methylpropane-2-sulfinamide (12.11 g, 100 mmol) in THF (100 mL) and the mixture stirred at 25° C. for 12 h. The reaction mixture was quenched by addition saturated aqueous NaHCO₃ solution (100 mL) and H₂O (100 mL) at 0° C. Then the reaction mixture was filtered and the filtrate was concentrated under reduced pressure. The residue was purified by column chromatography on silica gel (1-50% EtOAc/PE) to give the title compound as a yellow solid (4 g, 20%). LCMS m/z=204 [M+H]⁺.

### Step 2. Synthesis of tert-butyl(5-(3-((tert-butylsulfinyl)amino)tetrahydro-2H-pyran-3-yl)pyridin-3-yl)carbamate

n-BuLi (2.5 M, 9.15 mL) was added to a solution of tert-butyl N-(5-bromo-3-pyridyl)carbamate (2.5 g, 9.15 mmol) in THF (60 mL) at −78° C. and the mixture stirred at −78° C. for 0.5 h under N₂. A solution of (Z)—N-(dihydro-2H-pyran-3 (4H)-ylidene)-2-methylpropane-2-sulfinamide (Step 1, 4.65 g, 22.9 mmol) in THF (40 mL) was added to the mixture and stirred at −78° C. for 0.5 h under N₂. The reaction mixture was added to saturated aq·NH₄Cl (100 mL) at 0° C. and extracted with EtOAc (3×100 mL). The combined organics were dried (Na₂SO₄) and concentrated under reduced pressure. The residue was purified by MPLC (SiO₂, 0-100% EtOAc/PE) to give the title compound as a yellow solid (1.5 g, crude). LCMS m/z=398 [M+H]⁺.

### Step 3. Synthesis of 5-(3-aminotetrahydro-2H-pyran-3-yl)pyridin-3-amine

To a solution of tert-butyl(5-(3-((tert-butylsulfinyl)amino)tetrahydro-2H-pyran-3-yl)pyridin-3-yl)carbamate (Step 2, 1.4 g, 3.52 mmol) in HCl/EtOAc (15 mL) was stirred at 25° C. for 1 h.

The reaction mixture was filtered and the filtrate was concentrated under reduced pressure. The residue was purified by prep-HPLC-18 (1-25% MeCN) to give the title compound as a yellow solid (50 mg, 7%). LCMS m/z=194 [M+H]⁺.

### Intermediate A56. tert-butyl ((5-aminopyridin-3-yl)methyl)(methyl)carbamate

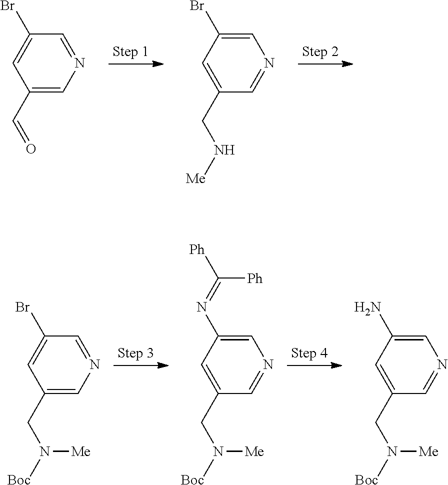 [SMILES: O=Cc1cncc(Br)c1 ; CNCc1cncc(Br)c1 ; CN(Cc1cncc(Br)c1)C(=O)OC(C)(C)C ; CN(Cc1cncc(N=C(c2ccccc2)c2ccccc2)c1)C(=O)OC(C)(C)C ; CN(Cc1cncc(N)c1)C(=O)OC(C)(C)C]

### Step 1. Synthesis of 1-(5-bromopyridin-3-yl)-N-methylmethanamine

The title compound was prepared from 5-bromonicotinaldehyde using an analogous method to that described for Intermediate A41 (Step 1). LCMS m/z=201 [M+H]⁺.

### Step 2. Synthesis of tert-butyl ((5-bromopyridin-3-yl)methyl)(methyl)carbamate

To a solution of 1-(5-bromopyridin-3-yl)-N-methylmethanamine (Ste p1, 5.20 g, 25.9 mmol) and (Boc)₂₀ (5.64 g, 25.9 mmol) in THF (52 mL) and H₂O (13 mL) was added Na₂CO₃ (5.48 g, 51.7 mmol) and the mixture stirred at 25° C. for 2 h. The reaction was diluted with H₂O (80 mL) and extracted with EtOAc (3×50 mL). The combined organics were dried (Na₂SO₄) and concentrated under reduced pressure and the residue was purified by MPLC (SiO₂, 50% EtOAc/PE) to give the title compound as a yellow oil (4.5 g, 58%). LCMS m/z=301 [M+H]⁺.

### Step 3. Synthesis of tert-butyl ((5-((diphenylmethylene)amino)pyridin-3-yl)methyl)(methyl)carbamate

To a solution of tert-butyl ((5-bromopyridin-3-yl)methyl)(methyl)carbamate (Step 2, 4.40 g, 14.61 mmol) and diphenylmethanimine (2.91 g, 16.07 mmol) in dioxane (70 mL) was added Pd₂ (dba)₃ (1.34 g, 1.46 mmol), Xantphos (1.69 g, 2.92 mmol) and Cs₂CO₃ (14.28 g, 43.83 mmol) and the mixture stirred at 100° C. for 3 h under N₂. The reaction mixture was filtered and the filtrate concentrated under reduced pressure and the residue purified by MPLC (SiO₂, 50% EtOAc/PE) to give the title compound was a yellow oil (3.50 g, 59%). LCMS m/z=402 [M+H]⁺.

### Step 4. Synthesis of tert-butyl ((5-aminopyridin-3-yl)methyl)(methyl)carbamate

To a solution of tert-butyl ((5-((diphenylmethylene)amino)pyridin-3-yl)methyl)(methyl)carbamate (Step 3, 3.40 g, 8.47 mmol) in MeOH (40 mL) was added NH₂OH·HCl (1.18 g, 16.94 mmol) and NaOAc (1.39 g, 16.94 mmol) and the mixture stirred at 25° C. for 1 h. The reaction mixture was concentrated under reduced pressure to remove solvent and extracted with EtOAc (3×50 mL). The combined organics were dried (Na₂SO₄) and concentrated under reduced and the residue purified by MPLC (SiO₂, EtOAc) to give the title compound as a yellow oil (2.00 g, 99%). LCMS m/z=402 [M+H]⁺.

### Intermediate A57A and A57B. tert-butyl(S)-(1-(5-aminopyridin-3-yl) but-3-yn-1-yl)carbamate and tert-butyl (R)-(1-(5-aminopyridin-3-yl) but-3-yn-1-yl)carbamate

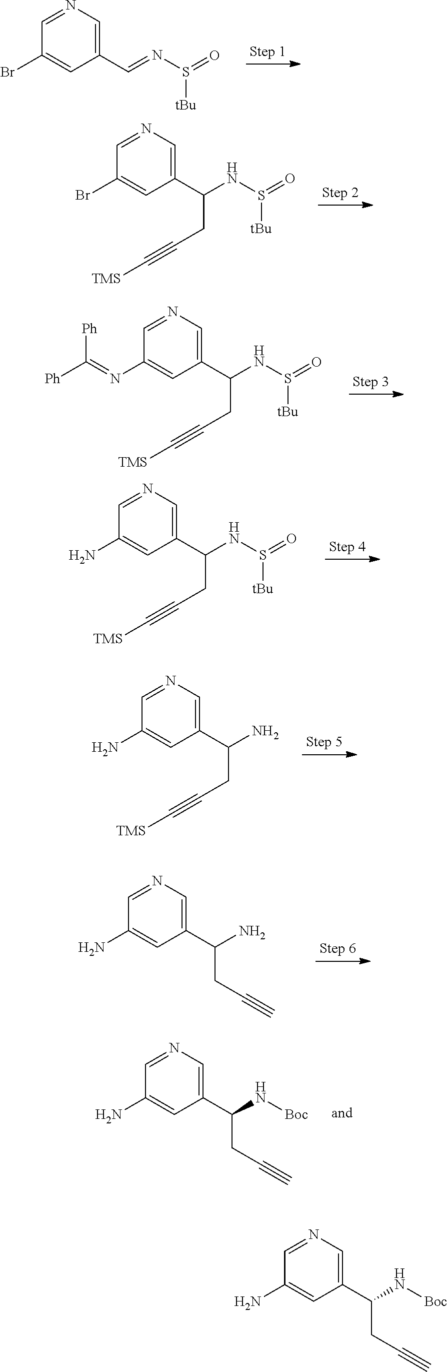

### Step 1. Synthesis of N-(1-(5-bromopyridin-3-yl)-4-(trimethylsilyl) but-3-yn-1-yl)-2-methylpropane-2-sulfinamide

Indigane (397 mg, 3.46 mmol) and 3-bromoprop-1-ynyl(trimethyl) silane (1.65 g, 8.64 mmol) were added to a solution of (E)-N-((5-bromopyridin-3-yl)methylene)-2-methylpropane-2-sulfinamide (Intermediate A7, Step 1 500 mg, 1.73 mmol) in THF (16 mL) and the mixture stirred at 60° C. for 7 h. The reaction mixture was filtered and the filtrate concentrated under reduced pressure and the residue purified by prep-HPLC-19 (45-75% MeCN) to give the title compound as a brown solid (930 mg, 33%). LCMS m/z=403 [M+H]⁺.

### Step 2. Synthesis of N-(1-(5-((diphenylmethylene)amino)pyridin-3-yl)-4-(trimethylsilyl) but-3-yn-1-yl)-2-methylpropane-2-sulfinamide

A mixture of N-(1-(5-bromopyridin-3-yl)-4-(trimethylsilyl) but-3-yn-1-yl)-2-methylpropane-2-sulfinamide (Step 1, 880 mg, 2.19 mmol), diphenylmethanimine (795 mg, 4.38 mmol), Cs₂CO₃ (1.43 g, 4.38 mmol), Xantphos (507 mg, 0.88 mmol) and Xantphos Pd G4 (422 mg, 0.44 mmol) in dioxane (10 mL) was degassed and purged with N₂ (×3) and the mixture stirred at 100° C. for 2 h under N₂. The reaction mixture was concentrated under reduced pressure and the residue purified by column chromatography (SiO₂, 0-25% EtOAc/PE) to afford the title compound as a yellow oil (1.5 g, crude). LCMS m/z=502 [M+H]⁺.

### Step 3. Synthesis of N-(1-(5-aminopyridin-3-yl)-4-(trimethylsilyl) but-3-yn-1-yl)-2-methylpropane-2-sulfinamide

To a solution of N-(1-(5-((diphenylmethylene)amino)pyridin-3-yl)-4-(trimethylsilyl) but-3-yn-1-yl)-2-methylpropane-2-sulfinamide (Step 2, 1.3 g, 2.59 mmol) in MeOH (10 mL) was added NH₂OH·HCl (270 mg, 3.89 mmol) and NaOAc (319 mg, 3.89 mmol) and the mixture stirred at 25° C. for 1 h. The residue was diluted with H₂O (50 mL) and extracted with EtOAc (3×40 mL). The combined organics were washed with brine (2×30 mL), dried (Na₂SO₄) and concentrated under reduced pressure to give the title compound as a white solid (1.1 g, crude). LCMS m/z=338 [M+H]⁺.

### Step 4. Synthesis of 5-(1-amino-4-(trimethylsilyl) but-3-yn-1-yl)pyridin-3-amine

A mixture of N-(1-(5-aminopyridin-3-yl)-4-(trimethylsilyl) but-3-yn-1-yl)-2-methylpropane-2-sulfinamide (Step 3, 1 g, 2.96 mmol) in HCl/EtOAc (7 mL) was stirred at 25° C. for 12 h. The reaction mixture was concentrated under reduced pressure to give the title compound as a white solid (650 mg, 94%). LCMS m/z=234 [M+H]⁺.

### Step 5. Synthesis of 5-(1-aminobut-3-yn-1-yl)pyridin-3-amine

To a solution of 5-(1-amino-4-(trimethylsilyl) but-3-yn-1-yl)pyridin-3-amine (600 mg, 2.57 mmol) in MeOH (2 mL) was added K₂CO₃ (1.07 g, 7.71 mmol) and the mixture stirred at 25° C. for 1 h. The reaction mixture was concentrated under reduced pressure to give the title compound as a yellow solid (500 mg, crude). LCMS m/z=162 [M+H]⁺.

### Step 6. Synthesis of tert-butyl(S)-(1-(5-aminopyridin-3-yl) but-3-yn-1-yl)carbamate and tert-butyl (R)-(1-(5-aminopyridin-3-yl) but-3-yn-1-yl)carbamate

Na₂CO₃ (657 mg, 6.20 mmol) was added to a solution of 5-(1-aminobut-3-yn-1-yl)pyridin-3-amine (Step 5, 500 mg, 3.10 mmol) and (Boc)₂ (677 mg, 3.1 mmol) in H₂O (3 mL) and MeOH (10 mL) and the mixture stirred at 25° C. for 1 h. The reaction mixture was concentrated under reduced pressure and the residue purified by column chromatography (SiO₂, 0-100% EtOAc/PE) followed by chiral SFC (DAICEL CHIRALPAK AD, 250×30 mm, 10 μm); 30% EtOH (0.1% NH₄OH) in CO₂) to afford:

Peak 1, Intermediate A57A: tert-butyl(S)-(1-(5-aminopyridin-3-yl) but-3-yn-1-yl)carbamate or tert-butyl (R)-(1-(5-aminopyridin-3-yl) but-3-yn-1-yl)carbamate (yellow solid, 70 mg, 8.6%). LCMS m/z=262 [M+H]⁺.

Peak 2, Intermediate A57B: tert-butyl(S)-(1-(5-aminopyridin-3-yl) but-3-yn-1-yl)carbamate or tert-butyl (R)-(1-(5-aminopyridin-3-yl) but-3-yn-1-yl)carbamate (yellow solid, 90 mg, 11%). LCMS m/z=262 [M+H]⁺.

### Intermediate A58. (1S,3R)-3-(((2,4-dimethoxybenzyl)amino)methyl)cyclohexan-1-amine or (1R,3S)-3-(((2,4-dimethoxybenzyl)amino)methyl)cyclohexan-1-amine

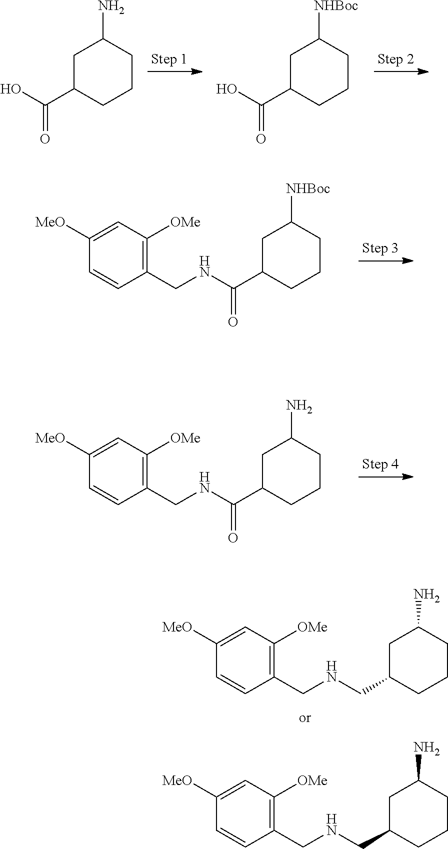 [SMILES: NC1CCCC(C(=O)O)C1 ; CC(C)(C)OC(=O)NC1CCCC(C(=O)O)C1 ; COc1ccc(CNC(=O)C2CCCC(NC(=O)OC(C)(C)C)C2)c(OC)c1 ; COc1ccc(CNC(=O)C2CCCC(N)C2)c(OC)c1 ; COc1ccc(CNC[C@H]2CCC[C@@H](N)C2)c(OC)c1 ; COc1ccc(CNC[C@@H]2CCC[C@H](N)C2)c(OC)c1]

### Step 1. Synthesis of 3-((tert-butoxycarbonyl)amino)cyclohexane-1-carboxylic acid

To the mixture of 3-aminocyclohexanecarboxylic acid (10 g, 69.8 mmol) and NaOH (4.19 g, 105 mmol) in dioxane (80 mL) and H₂O (30 mL) was added di-tert-butyl dicarbonate (22.86 g, 105 mmol) at 0° C. and the mixture stirred at 20° C. for 12 h. The mixture was concentrated under reduced pressure and the residue partitioned between EtOAc (100 mL) and H₂O (100 mL). The aqueous phase was washed with EtOAc (3×100 mL). The pH of the aqueous phase was adjusted to pH 5 with 1 M HCl. The solids were collected by filtration to afford the title compound as a white solid (14 g, 82%). LCMS m/z=243 [M+H]⁺.

### Step 2. Synthesis of tert-butyl(3-((2,4-dimethoxybenzyl)carbamoyl)cyclohexyl)carbamate

To a mixture of 3-((tert-butoxycarbonyl)amino)cyclohexane-1-carboxylic acid (Step 1, 10 g, 41.10 mmol) and (2,4-dimethoxyphenyl) methanamine (7.56 g, 45.21 mmol) in DMF (50 mL) was added DIPEA (10.62 g, 82.20 mmol) and HATU (23.44 g, 61.65 mmol) and the mixture stirred at 20° C. for 2 h. The reaction mixture was partitioned between EtOAc (20 mL) and H₂O (15 mL) and the aqueous phase extracted with EtOAc (3×20 mL). The combined organics were dried (Na₂SO₄) and concentrated under reduced pressure and the residue purified by MPLC (SiO₂, 0-100% EtOAc/PE) to give the title compound was a white solid (13 g, crude). ¹H NMR (400 MHz, CDCl₃): 7.07 (d, 1H), 6.38-6.33 (m, 2H), 5.87 (br s, 1H), 4.26 (d, 2H), 3.76 (s, 3H), 3.72 (s, 3H), 2.06-2.00 (m, 2H), 1.86 (br d, 1H), 1.77-1.69 (m, 2H), 1.35 (s, 9H), 1.32-1.24 (m, 2H), 1.04-0.95 (m, 1H).

### Step 3. Synthesis of 3-amino-N-(2,4-dimethoxybenzyl)cyclohexane-1-carboxamide

To a solution of tert-butyl(3-((2,4-dimethoxybenzyl)carbamoyl)cyclohexyl)carbamate (Step 2, 13 g, 33.1 mmol) in HCl/EtOAc (100 mL) was stirred at 25° C. for 1 h. The mixture was washed with saturated aqueous Na₂CO₃ (5 ml) until pH=9 and concentrated under reduced pressure. The residue was diluted with H₂O (20 mL) and extracted with DCM (3×20 mL), dried (Na₂SO₄) and concentrated under reduced pressure to give the title compound as a white solid (7.5 g, 77%). LCMS m/z=294 [M+H].

### Step 4. Synthesis of (1S,3R)-3-(((2,4-dimethoxybenzyl)amino)methyl)cyclohexan-1-amine or (1R,3S)-3-(((2,4-dimethoxybenzyl)amino)methyl)cyclohexan-1-amine

BH₃-DMS (10 M, 23.26 mL) was added to a solution of 3-amino-N-(2,4-dimethoxybenzyl)cyclohexane-1-carboxamide (Step 3, 6.8 g, 23.26 mmol) in THF (10 mL) at 0° C. and the mixture stirred at 60° C. for 2 h under N₂. The reaction mixture was quenched by addition of H₂O (5 mL) at 0° C. and concentrated under reduced pressure. The residue was purified by prep-HPLC-20 (1-13% MeCN) and prep-SFC (ChiralPak IH, 250×30 mm, 10 μm; 25% EtOH (+0.1% IPAm) in CO₂ to afford:

Peak 1, Intermediate A58: (1S,3R)-3-(((2,4-dimethoxybenzyl)amino)methyl)cyclohexan-1-amine or (1R,3S)-3-(((2,4-dimethoxybenzyl)amino)methyl)cyclohexan-1-amine (yellow oil, 1 g, 15%). LCMS m/z=280 [M+H]⁺.

### Intermediate A59. 5-aminopyridazine-3-carboxamide

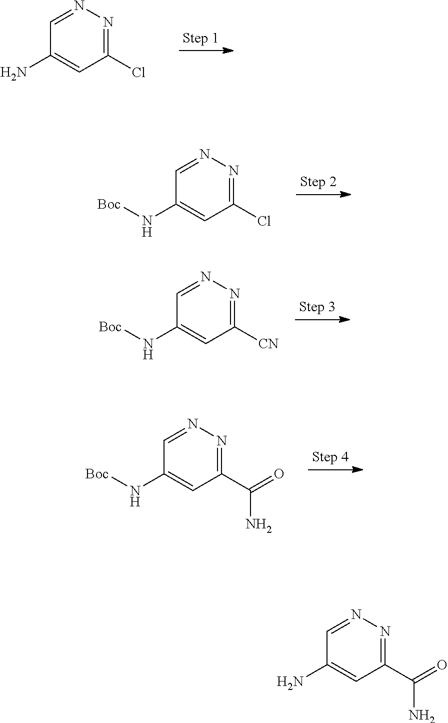 [SMILES: Nc1cnnc(Cl)c1 ; CC(C)(C)OC(=O)Nc1cnnc(Cl)c1 ; CC(C)(C)OC(=O)Nc1cnnc(C#N)c1 ; CC(C)(C)OC(=O)Nc1cnnc(C(N)=O)c1 ; NC(=O)c1cc(N)cnn1]

### Step 1. Synthesis of tert-butyl(6-chloropyridazin-4-yl)carbamate

To a solution of 6-chloropyridazin-4-amine (5 g, 38.6 mmol) in DCM (50 mL) was added (Boc)₂₀ (29.5 g, 135 mmol), DMAP (7.07 g, 57.9 mmol) and TEA (13.7 g, 135 mmol) and the mixture stirred at 25° C. for 1 h. The reaction mixture was diluted with H₂O (80 mL) and extracted with DCM (3×80 mL). The combined organics were dried (Na₂SO₄) and concentrated under reduced pressure and the residue purified by MPLC (SiO₂, 50% PE:EtOAc) to give the title compound as a yellow solid (6.40 g, crude). LCMS m/z=230 [M+H]⁺.

### Step 2. Synthesis of tert-butyl(6-cyanopyridazin-4-yl)carbamate

To a solution of tert-butyl(6-chloropyridazin-4-yl)carbamate (Step 1, 6.30 g, 27.4 mmol) in DMF (70 mL) was added BrettPhos Pd G3 (2.49 g, 2.74 mmol), Zn(CN)₂ (16.11 g, 137 mmol) and BRETTPHOS (1.47 g, 2.74 mmol) and the mixture stirred at 80° C. for 2 h under N₂. The mixture was diluted with H₂O (80 mL) and extracted with EtOAc (3×80 mL). The combined organics were dried (Na₂SO₄) and concentrated under reduced pressure. The residue was purified by MPLC (SiO₂, 50% EtOAc/PE) to give the title compound as a yellow oil (4.00 g, crude). LCMS m/z=221 [M+H]⁺.

### Step 3. Synthesis of tert-butyl(6-carbamoylpyridazin-4-yl)carbamate

To a solution of tert-butyl(6-cyanopyridazin-4-yl)carbamate (Part 2, 4 g, 18.2 mmol) in DMSO (40 mL) was added K₂CO₃ (5.02 g, 36.3 mmol) and the mixture stirred at 20° C. for 30 mins. To this was added H₂O₂ (20.6 g, 182 mmol) and stirred at 20° C. for 2 h. KOH (2.04 g, 36.3 mmol) was added and the resulting mixture stirred at 20° C. for 2 h. The reaction mixture was quenched by addition saturated aqueous Na₂SO₃ (5 mL) at 0° C., diluted with H₂O (50 mL) and extracted with EtOAc (3×50 mL). The combined organics were dried (Na₂SO₄) and concentrated under reduced pressure and the residue was purified by MPLC (SiO₂, 50% EtOAc/PE) to give the title compound as a white solid (350 mg, 8%). LCMS m/z=239 [M+H]⁺.

### Step 4. Synthesis of 5-aminopyridazine-3-carboxamide

A solution of tert-butyl(6-carbamoylpyridazin-4-yl)carbamate (330 mg, 1.39 mmol) in DCM (1 mL) and TFA (1 mL) was stirred at 25° C. for 1 h. The reaction mixture was filtered and the filtrate concentrated under reduced pressure to give the title compound as a yellow solid (160 mg, crude). LCMS m/z=139 [M+H]⁺.

### Intermediate A60. tert-butyl(S)-(1-(5-aminopyridin-3-yl) propyl)carbamate and tert-butyl (R)-(1-(5-aminopyridin-3-yl) propyl)carbamate

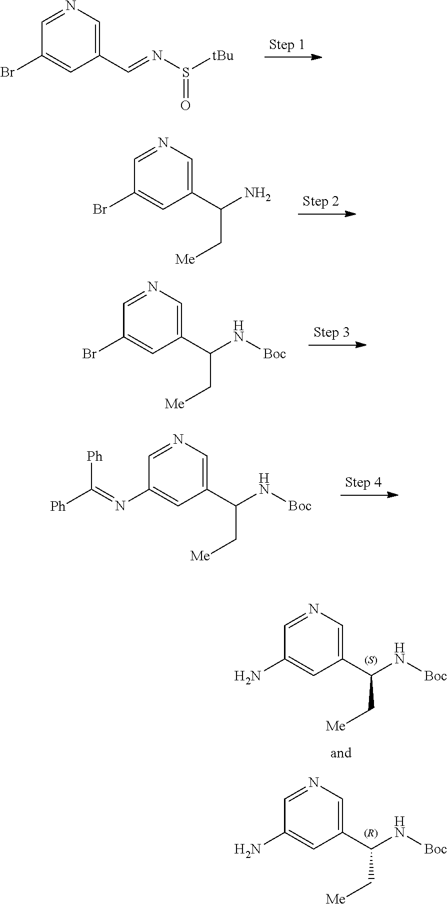

### Step 1. Synthesis of 1-(5-bromopyridin-3-yl)propan-1-amine hydrochloride

To a solution of (E)-N-((5-bromopyridin-3-yl)methylene)-2-methylpropane-2-sulfinamide (Intermediate A7 Step 1, 116 g, 401 mmol) in THF (1.2 L) was added EtMgBr (3 M, 140 mL) at −78° C. under N₂ and the mixture stirred at 0° C. for 2 h under N₂. The reaction mixture was quenched by addition saturated aqueous NH₄Cl solution (1 L) at 0° C., diluted with water (1 L) and extracted with EtOAc (4×1 L). The combined organics were dried (Na₂SO₄) and concentrated to give N-(1-(5-bromopyridin-3-yl) propyl)-2-methylpropane-2-sulfinamide as a yellow solid (500 g, crude). The solid was dissolved in EtOAc/HCl (1.2 L) and stirred for 12 h at 25° C. The reaction mixture was evaporated to dryness to afford the title compound as a yellow solid (560 g). LCMS m/z=215 [M+H]⁺.

### Step 2. Synthesis of tert-butyl(1-(5-bromopyridin-3-yl) propyl)carbamate

To a mixture of 1-(5-bromopyridin-3-yl)propan-1-amine hydrochloride (Step 1, 560 g, 2.60 mol) and (Boc)₂₀ (625 g, 2.86 mol) in THF (1.2 L) and H₂O (0.3 L) was added NaOH (1 M, 1 L) and the mixture stirred at 25° C. for 3 h. The reaction mixture was partitioned between EtOAc (1 L) and H₂O (1 L). The aqueous phase was extracted with EtOAc (3×1 L). The combined organics was dried (Na₂SO₄) and evaporated to dryness. The residue was purified by column chromatography on silica gel (25-33% EtOAc/PE) to give the title compound as a yellow solid (151 g, 18%). ¹H NMR (400 MHz, CDCl₃) δ: 8.60 (s, 1H), 8.49 (br s, 1H), 7.76 (s, 1H), 4.89 (d, 1H), 4.58 (br s, 1H), 1.87-1.75 (m, 2H), 1.45 (br s, 9H), 0.96 (t, 3H).

### Step 3. Synthesis of tert-butyl(1-(5-((diphenylmethylene)amino)pyridin-3-yl) propyl)carbamate

To the mixture of tert-butyl(1-(5-bromopyridin-3-yl) propyl)carbamate (Step 2, 130 g, 412 mmol) and diphenylmethanimine (82.22 g, 454 mmol) in dioxane (1500 mL) was added Pd₂ (dba)₃ (37.77 g, 41.24 mmol), Xantphos (47.73 g, 82.5 mmol) and Cs₂CO₃ (268.76 g, 825 mmol) and the mixture stirred at 100° C. for 3 h under N₂. The reaction mixture was filtered and the filtrate was concentrated under reduced pressure and the residue purified by column chromatography on silica gel (15-33% EtOAc/PE) to give the title compound as a brown oil (150 g, 87%). LCMS m/z=416 [M+H]⁺.

### Step 4. Synthesis of tert-butyl(S)-(1-(5-aminopyridin-3-yl) propyl)carbamate and tert-butyl (R)-(1-(5-aminopyridin-3-yl) propyl)carbamate

To the mixture of tert-butyl(1-(5-((diphenylmethylene)amino)pyridin-3-yl) propyl)carbamate (Step 3, 68 g, 164 mmol) in MeOH (800 mL) was added NH₂OH·HCl (22.74 g, 327 mmol) and NaOAc (33.56 g, 409 mmol) and the mixture stirred at 20° C. for 1 h. The reaction mixture was partitioned between EtOAc (1000 mL) and H₂O (800 mL) and the aqueous phase extracted with EtOAc (3×1000 mL). The combined organics were dried (Na₂SO₄) and concentrated under reduced pressure and the residue purified by MPLC (SiO₂, 0-100% EtOAc/PE) followed by chiral-SFC (DAICEL CHIRALPAK AD, 250×30 mm, 10 μm); 35% EtOH (0.1% NH₄OH) in CO₂) to afford.

Peak 2, Intermediate A60, tert-butyl(S)-(1-(5-aminopyridin-3-yl) propyl)carbamate or tert-butyl (R)-(1-(5-aminopyridin-3-yl) propyl)carbamate as a white solid (22 g, 53%). LCMS m/z=252 [M+H]⁺.

### Intermediate A61. tert-butyl(S)-2-(3-aminophenyl) pyrrolidine-1-carboxylate

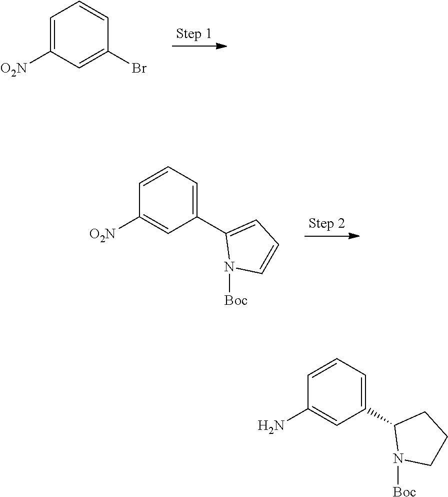 [SMILES: O=[N+]([O-])c1cccc(Br)c1 ; CC(C)(C)OC(=O)n1cccc1-c1cccc([N+](=O)[O-])c1 ; CC(C)(C)OC(=O)N1CCC[C@H]1c1cccc(N)c1]

### Step 1. Synthesis of tert-butyl 2-(3-nitrophenyl)-1H-pyrrole-1-carboxylate

To a solution of 1-bromo-3-nitrobenzene (4.4 g, 21.8 mmol) and tert-butyl 2-(4,4,5,5-tetramethyl-1,3,2-dioxaborolan-2-yl) pyrrole-1-carboxylate (6.39 g, 21.8 mmol) in DME (15 mL)/H₂O (4 mL) was added Pd(PPh₃)₄ (2.52 g, 2.18 mmol) and Na₂CO₃ (4.62 g, 43.6 mmol) and the mixture stirred at 90° C. for 2 h under N₂. The reaction mixture was filtered and the filtrate was concentrated under reduced pressure and the residue purified by flash column chromatography (SiO₂, 0-16% EtOAc/PE) to give the title compound as a yellow oil (5.8 g, 92%).

### Step 2. Synthesis of tert-butyl(S)-2-(3-aminophenyl) pyrrolidine-1-carboxylate and tert-butyl(R)-2-(3-aminophenyl) pyrrolidine-1-carboxylate

To a mixture of tert-butyl 2-(3-nitrophenyl)-1H-pyrrole-1-carboxylate (5.8 g, 20.12 mmol) in MeOH (50 mL) was added Pd/C (4.7 g, 10% purity) slowly and the mixture stirred at 60° C. for 8 h under H₂ (50 psi). The reaction mixture was filtered and the filtrate was concentrated under reduced pressure and the residue purified by chiral SFC (Lux Cellulose-2, 250×30 mm; 10 μm; 25% EtOH (+0.1% NH₄OH) in CO₂) to afford:

Peak 1, Intermediate A61: tert-butyl(S)-2-(3-aminophenyl) pyrrolidine-1-carboxylate or tert-butyl(R)-2-(3-aminophenyl) pyrrolidine-1-carboxylate as a white solid (1.3 g, 24%). LCMS m/z=163 [M-Boc+H]⁺.

### Intermediate A62. tert-butyl(1-(5-aminopyridin-3-yl)cyclopropyl)carbamate

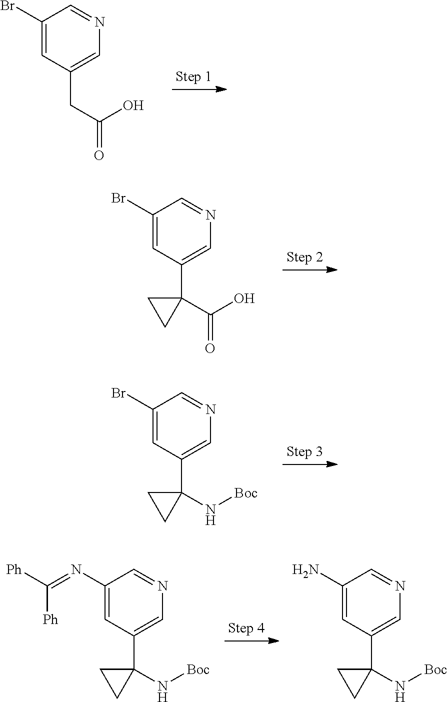 [SMILES: O=C(O)Cc1cncc(Br)c1 ; O=C(O)C1(c2cncc(Br)c2)CC1 ; CC(C)(C)OC(=O)NC1(c2cncc(Br)c2)CC1 ; CC(C)(C)OC(=O)NC1(c2cncc(N=C(c3ccccc3)c3ccccc3)c2)CC1 ; CC(C)(C)OC(=O)NC1(c2cncc(N)c2)CC1]

### Step 1. Synthesis of 1-(5-bromopyridin-3-yl)cyclopropane-1-carboxylic acid

The title compound was prepared from 2-(5-bromopyridin-3-yl) acetic acid using an analogous 3-Step method as described for Intermediate A39, Steps 1-3. LCMS m/z=242 [M+H]⁺.

### Step 2. Synthesis of tert-butyl(1-(5-bromopyridin-3-yl)cyclopropyl)carbamate

To a solution of 1-(5-bromopyridin-3-yl)cyclopropane-1-carboxylic acid (Step 1, 600 mg, 2.48 mmol) in t-BuOH (24 mL) was added DPPA (1.02 g, 3.72 mmol), 4 Å molecular sieve (600 mg) and triethylamine (376 mg, 3.72 mmol) and the mixture stirred at 25° C. for 1 h and at 100° C. for 3 h. The reaction mixture was diluted with water (60 mL) and extracted with ethyl acetate (3×50 mL). The combined organics were dried (Na₂SO₄) and concentrated under reduced pressure and the residue purified by silica gel chromatography (0-25% EtOAc/PE) to afford the title compound as an off-white solid (730 mg, 94%). LCMS m/z=313 [M+H]⁺.

### Step 3. Synthesis of tert-butyl(1-(5-((diphenylmethylene)amino)pyridin-3-yl)cyclopropyl)carbamate

A mixture of tert-butyl(1-(5-bromopyridin-3-yl)cyclopropyl)carbamate (Step 2, 365 mg, 2.01 mmol), diphenylmethanimine (600 mg, 1.92 mmol), Cs₂CO₃ (1.87 g, 5.75 mmol), XantPhos (222 mg, 383 μmol) and Pd₂ (dba)₃ (175 mg, 192 μmol) in dioxane (15 mL) was degassed and purged with nitrogen (3×) and the mixture stirred at 100° C. for 3 h under N₂. The reaction mixture was concentrated under reduced and the residue purified by chromatography column 0-25% EtOAc/PE) to afford the title compound as a yellow oil (590 mg, 74%). LCMS m/z=414 [M+H]⁺.

### Step 4. Synthesis of tert-butyl(1-(5-aminopyridin-3-yl)cyclopropyl)carbamate

To a solution of tert-butyl(1-(5-((diphenylmethylene)amino)pyridin-3-yl)cyclopropyl)carbamate (Step 3, 590 mg, 1.43 mmol) in MeOH (20 mL) was added hydroxylamine hydrochloride (3.80 g, 54.7 mmol) and sodium acetate (5.80 g, 70.7 mmol) and the mixture stirred at 25° C. for 2 h. The reaction mixture was added to saturated aqueous sodium bicarbonate (100 mL) and concentrated under reduced pressure to remove MeOH and the residue extracted with EtOAc (3×50 mL). The combined organics were dried (Na₂SO₄), concentrated under reduced pressure and the residue purified by silica gel chromatography column (0-100% EtOAc/PE) to afford the title compound as an off-white solid (330 mg, 93%). LCMS m/z=250 [M+H]⁺.

### Intermediate A63. tert-butyl(4-(5-aminopyridin-3-yl)tetrahydro-2H-pyran-4-yl)carbamate

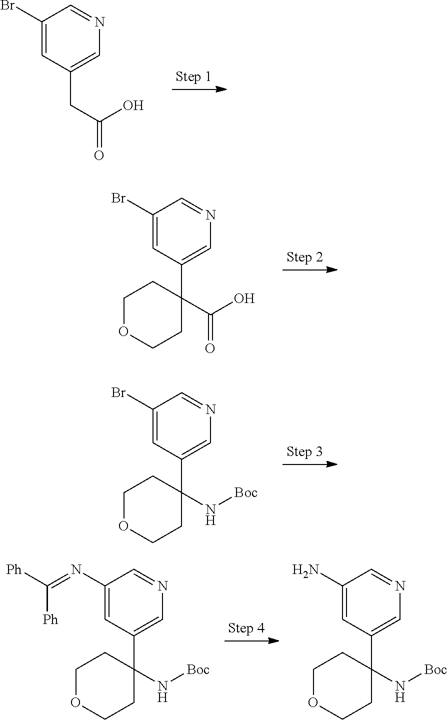

The title compound was prepared in an analogous 4-Step method as described for Intermediate A62. LCMS m/z=294 [M+H].

### Intermediate A64. tert-butyl(2-amino-6-methoxyphenethyl)(methyl)carbamate

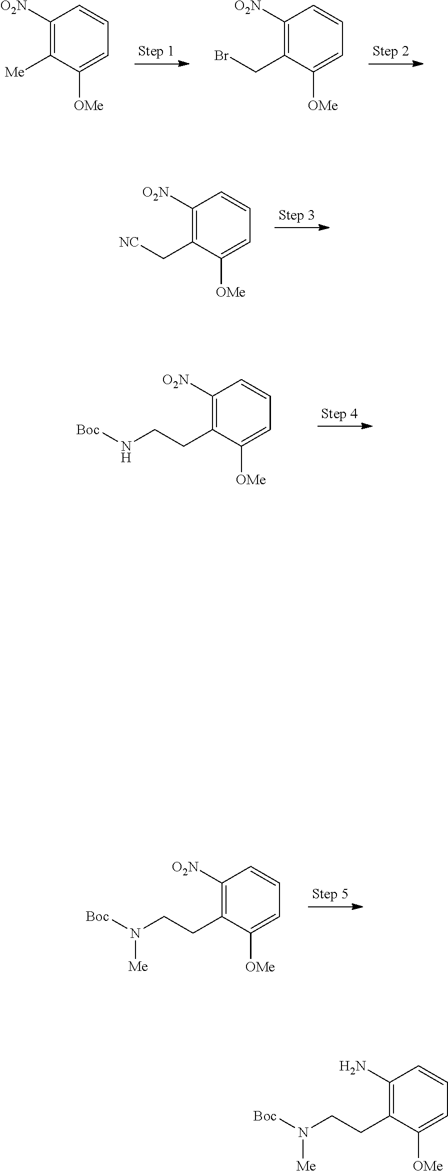 [SMILES: COc1cccc([N+](=O)[O-])c1C ; COc1cccc([N+](=O)[O-])c1CBr ; COc1cccc([N+](=O)[O-])c1CC#N ; COc1cccc([N+](=O)[O-])c1CCNC(=O)OC(C)(C)C ; COc1cccc([N+](=O)[O-])c1CCN(C)C(=O)OC(C)(C)C ; COc1cccc(N)c1CCN(C)C(=O)OC(C)(C)C]

### Step 1. Synthesis of 2-(bromomethyl)-1-methoxy-3-nitrobenzene

To a solution of 1-methoxy-2-methyl-3-nitrobenzene (3 g, 17.95 mmol) in tetrachloromethane (60 mL) was added N-bromosuccinimide (3.51 g, 19.74 mmol) and benzoyl peroxide (435 mg, 1.79 mmol) and the mixture stirred at 80° C. for 12 h. The reaction mixture was diluted with water (50 mL) and was extracted with ethyl acetate (3×60 mL). The combined organics were dried (Na₂SO₄) and concentrated under reduced pressure and the residue purified by silica gel chromatography (0-20% EtOAc/PE) to afford the title compound as a yellow solid (4.3 g, 97%). ¹H NMR (400 MHz, CDCl₃): 7.55 (d, 1H), 7.43 (t, 1H), 7.16 (d, 1H), 4.84 (s, 2H), 3.99 (s, 3H).

### Step 2. Synthesis of 2-(2-methoxy-6-nitrophenyl) acetonitrile

To a solution of 2-(bromomethyl)-1-methoxy-3-nitrobenzene (Step 1, 4.3 g, 17.48 mmol) in acetonitrile (60 mL) was added lithium hydroxide monohydrate (880 mg, 20.97 mmol) at 0° C. followed by trimethylsilyl cyanide (2.60 g, 26.21 mmol) and the mixture stirred at 25° C. for 16 h. The reaction mixture was diluted with water (50 mL) and was extracted with EtOAc (3×60 mL). The combined organics were dried (Na₂SO₄) and concentrated under reduced pressure. The residue was purified by silica gel chromatography (0-60% EtOAc/PE) to afford the title compound as a yellow solid (3.1 g, 92%).

### Step 3. Synthesis of tert-butyl(2-methoxy-6-nitrophenethyl)carbamate

To a solution of 2-(2-methoxy-6-nitrophenyl) acetonitrile (Step 2, 1.5 g, 7.81 mmol) in THF (80 mL) was added borane-THE complex (1 M, 23.42 mL) at 25° C. under N₂ and the mixture was heated at 60° C. for 16 h. The reaction mixture was quenched by addition MeOH (5 mL) and stirred for 0.5 hour. To this was added HCl (1M, 10 mL) and the mixture was heated to 60° C. and stirred for 0.5 h. The mixture was diluted with H₂O (100 mL) and extracted with EtOAc (3×80 mL). The combined organics were discarded. The aqueous phase was basified by addition saturated aqueous sodium bicarbonate to pH=8~9 and used directly. To this was added di-tert-butyl dicarbonate (1.90 g, 8.71 mmol) and the mixture was stirred at 25° C. for 1 h. The reaction mixture was extracted with EtOAc (3×60 mL) and the combined organics dried (Na₂SO₄) and concentrated under reduced pressure. The residue was purified by silica gel chromatography (0-16% EtOAc/PE) to afford the title compound as a yellow solid (1.5 g, 66%). ¹H NMR (400 MHz, CDCl₃): 7.39 (d, 1H), 7.32 (t, 1H), 7.08 (d, 1H), 4.79 (s, 1H), 3.92 (s, 3H), 3.44 (t, 2H), 3.01 (t, 2H), 1.41 (s, 9H).

### Step 4. Synthesis of tert-butyl(2-methoxy-6-nitrophenethyl)(methyl)carbamate

To a solution of tert-butyl(2-methoxy-6-nitrophenethyl)carbamate (Step 3, 500 mg, 1.69 mmol) in THF (20 mL) was added NaH (135 mg, 3.37 mmol, 60% purity) at 25° C. and the mixture stirred at 25° C. for 0.5 h and MeI (479 mg, 3.37 mmol) added. The resulting mixture was stirred at 40° C. for 15.5 h. Additional NaH (135 mg, 3.37 mmol, 60% purity) was added to the reaction mixture at 25° C., and then heated to 40° C. and stirred for another 15.5 h. The reaction mixture was quenched with H₂O (30 mL) and extracted with EtOAc (3×20 mL). The combined organics were dried (Na₂SO₄) and concentrated under reduced pressure and the residue purified by chromatography (SiO₂, 0-70% MeCN/H₂O (0.1% HCl)) to afford the title compound as a yellow oil (210 mg, 40%). ¹H NMR (400 MHz, CDCl₃): 7.40 (d, 1H), 7.32 (t, 1H), 7.06 (d, 1H), 3.90 (s, 3H), 3.49 (t, 2H), 3.03 (t, 2H), 2.90 (s, 3H), 1.39 (s, 9H).

### Step 5. Synthesis of tert-butyl(2-amino-6-methoxyphenethyl)(methyl)carbamate

To a solution of tert-butyl(2-methoxy-6-nitrophenethyl)(methyl)carbamate (Step 4, 210 mg, 0.677 mmol) in EtOH (9 mL) and H₂O (3 mL) was added iron (189 mg, 3.38 mmol) and ammonium chloride (189 mg, 3.38 mmol) and the mixture stirred at 70° C. for 2 h. The mixture was filtered and concentrated under reduced pressure. The residue was diluted with H₂O (20 mL) and extracted with EtOAc (3×20 mL). The combined organics were washed with brine (60 mL), dried (Na₂SO₄) and concentrated under reduced pressure. The residue was purified by column chromatography (SiO₂, 33-50% EtOAc/PE) to afford the title compound as a colourless oil (170 mg, 75%). ¹H NMR (400 MHz, DMSO-d₆): 6.86 (t, 1H), 6.29 (d, 1H), 6.20 (d, 1H), 5.32-4.59 (m, 2H), 3.67 (s, 3H), 3.25-3.08 (m, 2H), 2.83-2.73 (m, 3H), 2.70-2.59 (m, 2H), 1.42-1.29 (m, 9H).

### Intermediate A65. tert-butyl 2-(3-aminophenyl)-5-(hydroxymethyl) pyrrolidine-1-carboxylate

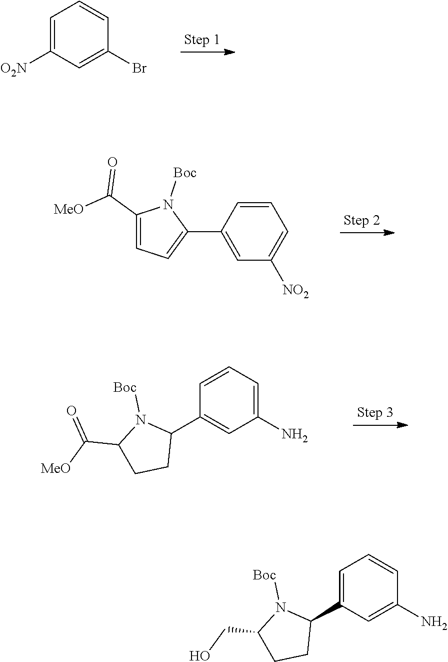 [SMILES: O=[N+]([O-])c1cccc(Br)c1 ; COC(=O)c1ccc(-c2cccc([N+](=O)[O-])c2)n1C(=O)OC(C)(C)C ; COC(=O)C1CCC(c2cccc(N)c2)N1C(=O)OC(C)(C)C ; CC(C)(C)OC(=O)N1[C@@H](CO)CC[C@@H]1c1cccc(N)c1]

### Step 1. Synthesis of 1-(tert-butyl)₂-methyl 5-(3-nitrophenyl)-1H-pyrrole-1,2-dicarboxylate

A solution of (1-(tert-butoxycarbonyl)-5-(methoxycarbonyl)-1H-pyrrol-2-yl) boronic acid (1.004 g, 3.96 mmol), 1-bromo-3-nitrobenzene (667 mg, 3.30 mmol), PdCl₂ (dppf)-DCM adduct (139 mg, 0.165 mmol), and tBuXPhos (107 mg, 0.248 mmol) in dioxane (18 mL) and saturated aqueous Na₂CO₃ (4.5 mL) was purged with N₂ for 10 min and heated to 80° C. for 3 h. The reaction was cooled, filtered through Celite and washed with 75 mL EtOAc. The filtrate was concentrated by rotary evaporation and the residue purified by flash chromatography (SiO₂, 0-70% EtOAc/Hex) to afford the title compound as a yellow oil (336 mg, 28%). LCMS m/z=369 [M+Na]⁺.

### Step 2. 1-(tert-butyl)₂-methyl 5-(3-aminophenyl) pyrrolidine-1,2-dicarboxylate

To a solution of 1-(tert-butyl)₂-methyl 5-(3-nitrophenyl)-1H-pyrrole-1,2-dicarboxylate (Step 1, 719 mg, 2.07 mmol) in MeOH (10 mL), added Pt/C (5 wt %, 661 mg, 0.17 mmol). The flask was sparged with H₂ for 10 min and then stirred at RT under a balloon atmosphere of H₂ for 19 h. The reaction mixture was then filtered through Celite and washed with EtOAc (30 mL) and concentrated by rotary evaporation. The residue was purified by flash chromatography (ISCO, 24 g silica, 0-70% EtOAc/Hex) provided the title compound as viscous yellow oil that partially solidified under vacuum (504 mg, 76%). LCMS m/z=343 [M+Na]⁺.

### Step 3. tert-butyl 2-(3-aminophenyl)-5-(hydroxymethyl) pyrrolidine-1-carboxylate

LiBH₄ (160 mg, 7.8 mmol) was added to 1-(tert-butyl)₂-methyl 5-(3-aminophenyl) pyrrolidine-1,2-dicarboxylate (Step 2, 417 mg, 1.3 mmol) in MeOH (13 mL) at RT and stirred for 15 min and then 50° C. for 3 days. Additional LiBH₄ (166 mg, 7.8 mmol) was added and heating continued at 50° C. for 19 h. The reaction mixture was poured into aqueous ammonium chloride and extracted with EtOAc (3×30 mL). The combined organics were washed twice with water, brine, dried (MgSO₄) and concentrated by rotary evaporation. The residue was purified by flash chromatography (ISCO, 24 g silica, 0-100% EtOAc/Hex) to afford the title compound as a white solid (112 mg, 29%). LCMS m/z=315 [M+Na]⁺. ¹H-NMR analysis showed a single diastereomer, but the identity could not be determined due to peak broadening. The trans isomer is most likely based on literature precedent for the synthetic route employed (see: Kaiser and Muchowski, J. Org. Chem, 1984, 49 (22), 4203-4209).

### Intermediate A66. tert-butyl ((5-aminopyridin-3-yl)methyl) (2-(methylsulfonyl)ethyl)carbamate

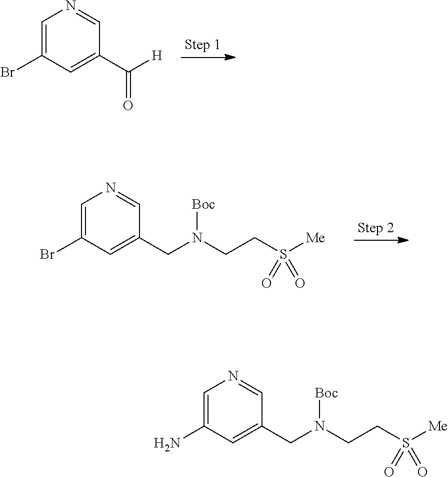 [SMILES: O=Cc1cncc(Br)c1 ; CC(C)(C)OC(=O)N(CCS(C)(=O)=O)Cc1cncc(Br)c1 ; CC(C)(C)OC(=O)N(CCS(C)(=O)=O)Cc1cncc(N)c1]

### Step 1. Synthesis of tert-butyl ((5-bromopyridin-3-yl)methyl) (2-(methylsulfonyl)ethyl)carbamate

To a suspension of 5-bromonicotinaldehyde (563 mg, 3.0 mmol) and 2-(methylsulfonyl) ethan-1-amine hydrochloride (492 mg, 3.0 mmol) in DCE (10 mL) were added sequentially DIPEA (0.630 mL, 3.6 mmol) and NaBH (OAc)₃ (899 mg, 4.2 mmol) and the resulting mixture stirred at RT for 7 h. The reaction was poured into saturated aqueous sodium bicarbonate and extracted with EtOAc (3×30 mL). The combined organics were washed with brine, dried (MgSO₄) and concentrated by rotary evaporation to afford N-((5-bromopyridin-3-yl)methyl)-2-(methylsulfonyl) ethan-1-amine. The crude amine was dissolved in THF (12 mL) and Boc₂O (722 mg, 3.15 mmol) added and the reaction stirred at RT for 2 h. The reaction was poured into aqueous sodium bicarbonate and extracted with EtOAc (3×30 mL). The combined organics were washed with brine, dried (MgSO₄) and concentrated by rotary evaporation. The residue was purified by flash chromatography (ISCO, 24 g silica, 0-100% EtOAc/hex) to afford the title compound as a clear yellow oil (423 mg, 36% yield over 2 steps). LCMS m/z=393 [M+H]⁺.

### Step 2. Synthesis of tert-Butyl ((5-aminopyridin-3-yl)methyl) (2-(methylsulfonyl)ethyl)carbamate

To a suspension of tert-butyl ((5-bromopyridin-3-yl)methyl) (2-(methylsulfonyl)ethyl)carbamate (Part 1, 414 mg, 1.05 mmol) and cuprous oxide (68 mg, 0.42 mmol) in NMP (4 mL) was added aqueous ammonium hydroxide (28-30%, 1.5 mL, 10.5 mmol). The flask was sealed and heated to 100° C. for 21 h. The reaction was cooled to RT, poured into water and extracted EtOAc (4×10 mL). The combined organics were washed with water (×2), brine, dried (MgSO₄) and concentrated by rotary evaporation. The residue was purified by flash chromatography (ISCO 12 g silica, 0-100% EtOAc/hex then 10% MeOH/DCM) to afford the title compound as a highly viscous, clear yellow oil (234 mg, 67%). ¹H-NMR analysis showed a roughly 40:60 mixture of rotamers. LCMS m/z=330 [M+H]⁺.

### Intermediate A67. (R)-3-fluoro-5-(1-(methylamino)ethyl) aniline

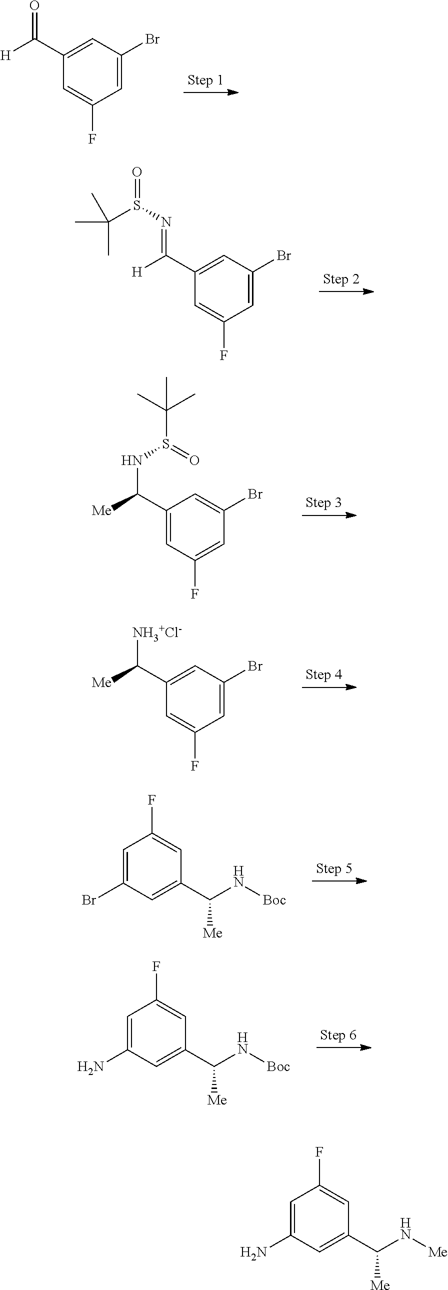

### Step 1. Synthesis of (S,E)-N-(3-bromo-5-fluorobenzylidene)-2-methylpropane-2-sulfinamide

To a solution of (S)-2-methylpropane-2-sulfinamide (4.41 g, 37.5 mmol) and 3-bromo-5-fluorobenzaldehyde (2.538 g, 12.5 mmol) in DCM (25 mL) at RT was added sequentially PPTS (315 mg, 1.25 mmol) and MgSO₄ (7.31 g, 67.5 mmol) and the reaction mixture stirred at RT for 17 h. The reaction was filtered through a 1-inch pad of silica gel and Celite, washing with 100 mL DCM and 20 mL 10% MeOH/DCM. The combined organics were evaporated to dryness and the residue purified by flash chromatography (ISCO, 80 g silica, 0-70% EtOAc/hex) to afford the title compound as a white solid (3.094 g, 81%). LCMS m/z=306 [M+H]⁺.

### Step 2. Synthesis of (S)—N—((R)-1-(3-bromo-5-fluorophenyl)ethyl)-2-methylpropane-2-sulfinamide

To a dry flask under N₂ atmosphere was added a solution of (S,E)-N-(3-bromo-5-fluorobenzylidene)-2-methylpropane-2-sulfinamide (Step 1, 1.256 g, 4.0 mmol) in anhydrous DCM (25 mL). The solution was cooled to −78° C. and MeMgBr solution (3 M in Et2O, 3.00 mL, 9.0 mmol) was added dropwise over 4 mim and the reaction left in the cold bath to warm to RT overnight. The reaction was quenched with saturated aqueous ammonium chloride (10 mL), diluted with DCM (100 mL) and poured into water (100 mL). The aqueous phase was extracted with DCM (2×50 mL). The combined organics were washed with brine, dried (MgSO₄) and concentrated by rotary evaporation. The residue was purified by flash chromatography (ISCO, 40 g silica, 0-100% EtOAc/hex) to provide the title compound as a clear, colourless oil (1.003 g, 77%). LCMS m/z=322 [M+H]⁺.

### Step 3. Synthesis of (R)-1-(3-bromo-5-fluorophenyl) ethan-1-amine hydrochloride

4M HCl dioxane solution (1.55 mL, 6.2 mmol) was added to a solution of (S)—N—((R)-1-(3-bromo-5-fluorophenyl)ethyl)-2-methylpropane-2-sulfinamide (Step 2, 1.003 g, 3.11 mmol) in MeOH (6.5 mL) at RT and the reaction was stirred at RT for 45 min. The mixture was concentrated under reduced pressure until the material was just fully soluble and then poured into Et₂O (100 mL). A white ppt crashed out and the mixture left to stand for 15 min. The precipitate was collected by filtration, washing with 100 mL diethyl ether and dried to provide the title compound as a white solid (701 mg, 88%). LCMS m/z=218 [M+H]⁺.

### Step 4. Synthesis of tert-butyl (R)-(1-(3-bromo-5-fluorophenyl)ethyl)carbamate

To a suspension of (R)-1-(3-bromo-5-fluorophenyl) ethan-1-amine hydrochloride (Step 3, 688 mg, 2.70 mmol) in DCM (20 mL) was added TEA (0.60 mL, 4.32 mmol). The reaction mixture was stirred at RT for 30 min and Boc₂O (568 mg, 2.65 mmol) added in a single portion and the reaction stirred at RT for 3 h. The reaction was then diluted with DCM (75 mL) and washed successively with dilute aqueous HCl, saturated aqueous sodium bicarbonate, water, and brine. The organics was dried (MgSO₄) and concentrated by rotary evaporation. Flash chromatography (ISCO, 12 g silica, 0-80% EtOAc/hexanes) provided the title compound as a white solid (731 mg, 85%). LCMS m/z=340 [M+Na]⁺.

### Step 5. Synthesis of tert-butyl (R)-(1-(3-amino-5-fluorophenyl)ethyl)carbamate

To a suspension of tert-butyl (R)-(1-(3-bromo-5-fluorophenyl)ethyl)carbamate (Step 4, 162 mg, 0.51 mmol), Cs₂CO₃ (240 mg, 0.71 mmol), Pd₂ (dba)₃ (34.5 mg, 0.026 mmol), and BINAP (32.2 mg, 0.51 mmol) in anhydrous toluene (2 mL) was added benzophenone imine (0.110 mL, 0.61 mmol) and the mixture was purged with nitrogen for 10 min and then heated to 80° C. for 15 h. The reaction was cooled to RT and filtered through Celite and washed with EtOAc. Flash chromatography (ISCO, 12 g silica, 0-50% EtOAc/Hex) afford the intermediate tert-butyl (R)-(1-(3-((diphenylmethylene)amino)-5-fluorophenyl)ethyl)carbamate (130 mg) enriched to ~90% purity. A solution of tert-butyl (R)-(1-(3-((diphenylmethylene)amino)-5-fluorophenyl)ethyl)carbamate (130 mg, 0.31 mmol) in MeOH (5 mL) was cooled on ice and hydroxylamine hydrochloride (64.8 mg, 0.93 mmol) and sodium acetate (126 mg, 1.55 mmol) added sequentially. The reaction was removed from ice and stirred at RT for 4 h. The reaction was poured into aqueous ammonium chloride and extracted with EtOAc (3×20 mL). The combined organics were washed with brine, dried (MgSO₄) and concentrated by rotary evaporation. The residue was purified by flash chromatography (ISCO, 4 g silica, 0-100% EtOAc/hex) to afford the title compound as a clear yellow oil (72.1 mg, 55% yield over 2 steps). LCMS m/z=255 [M+H]⁺.

### Step 6. Synthesis of (R)-3-fluoro-5-(1-(methylamino)ethyl) aniline

A solution of tert-butyl (R)-(1-(3-amino-5-fluorophenyl)ethyl)carbamate (Step 5, 69.1 mg, 0.27 mmol) in anhydrous THF (5 mL) was cooled on ice. LiAlH₄ (54 mg, 1.35 mmol) was added in several portions. The reaction was removed from the ice bath and heated to 65° C. for 4 h. The reaction was cooled to RT diluted with EtOAc (5 mL) and cautiously quenched with water (200 μL). 1 M aqueous NaOH (1 mL) was added and the mixture was stirred at RT for 5 min. Excess MgSO₄ was added and the mixture filtered through a 1-inch pad of MgSO₄, washing with EtOAc (50 mL). The filtrate was concentrated to provide the title compound as a clear yellow oil (40.3 mg, 89%). ¹H NMR (500 MHz, CDCl₃): 6.47-6.37 (m, 2H), 6.26 (dt, 1H), 3.93-3.59 (m, 2H), 3.53 (q, 1H), 2.31 (s, 3H), 1.32 (d, 3H).

### Intermediate A68. tert-butyl 3-(5-aminopyridin-3-yl) morpholine-4-carboxylate

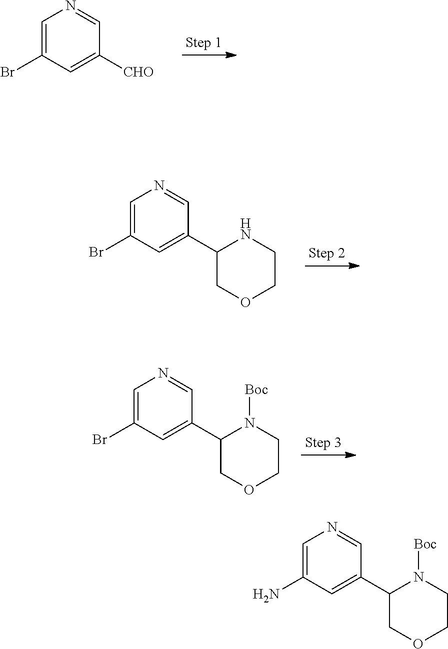 [SMILES: O=Cc1cncc(Br)c1 ; Brc1cncc(C2COCCN2)c1 ; CC(C)(C)OC(=O)N1CCOCC1c1cncc(Br)c1 ; CC(C)(C)OC(=O)N1CCOCC1c1cncc(N)c1]

### Step 1. Synthesis of 3-(5-bromopyridin-3-yl) morpholine

To a solution of the 2-((tributylstannyl) methoxy) ethan-1-amine (1092 mg, 3.00 mmol) in DCM (15 mL) was added 5-bromonicotinaldehyde (558 mg, 3.0 mmol) and 4 Å molecular sieves (350 mg) under an inert atmosphere at ambient temperature. The reaction mixture was stirred for 2 h and filtered through a pad of Celite rinsing with dichloromethane (50 mL). The filtrate was concentrated under reduced pressure to afford 1-(5-bromopyridin-3-yl)-N-(2-((tributylstannyl) methoxy)ethyl) methanimine.

Separately, anhydrous copper (II) trifluoromethanesulfonate (1085 mg, 3.0 mmol) was added to a solution of 2,6-lutidine (0.35 mL) in 1,1,1,3,3,3-hexafluoropropan-2-ol (12 mL) and stirred at RT for 1 h. A solution of 1-(5-bromopyridin-3-yl)-N-(2-((tributylstannyl) methoxy)ethyl) methanimine in dry DCM (48 mL) was added in one portion and the resulting mixture stirred at RT for 36 h. The reaction was quenched with a mixture of NaHCO₃ (24 mL) and 10% aqueous NH₄OH (12 mL) and stirred vigorously for 15 min. The layers were then separated and the aqueous layer extracted with DCM (3×10 mL). The combined organics were then washed with water (3×10 mL), brine (5 mL), dried (Na₂SO₄) and evaporated to dryness. The residue was purified by flash chromatography (ISCO, 24 g silica, 0-15% MeOH (w/5% NH₄OH) in DCM) provided the title compound as a yellow solid (351 mg, 48%). LCMS m/z=244 [M+H]⁺.

### Step 2. Synthesis of tert-butyl 3-(5-bromopyridin-3-yl) morpholine-4-carboxylate

To a solution of 3-(5-bromopyridin-3-yl) morpholine (Step 1, 350 mg, 1.44 mmol) in DCM (2.9 mL) was added (Boc)₂₀ (368 μl, 1.58 mmol), DMAP (8.8 mg, 0.066 mmol) and TEA (301 μl, 2.16 mmol) and the mixture stirred at ambient temperature for a 4 h. The mixture was poured into water (10 mL) and the aqueous layer extracted with DCM (3×10 mL). The combined organics were washed sat aq NH₄Cl (10 mL), brine (10 mL), dried (Na₂SO₄) and evaporated to dryness. The residue was purified by flash chromatography (ISCO, 24 g silica, 10-50% EtOAc/Hex) to afford the title compound as a white solid (481 mg, 97%). LCMS m/z=344 [M+H]⁺.

### Step 3. Synthesis of tert-butyl 3-(5-aminopyridin-3-yl) morpholine-4-carboxylate

To a suspension of tert-butyl 3-(5-bromopyridin-3-yl) morpholine-4-carboxylate (Step 2, 480 mg, 1.399 mmol), Cs₂CO₃ (683 mg, 2.098 mmol), Pd₂ (dba)₃ (64 mg, 0.070 mmol) and BINAP (87 mg, 0.140 mmol) in anhydrous toluene (5.6 mL) was added benzophenone imine (290 μL, 1.68 mmol). The mixture was sparged with N₂ for 10 min and then heated to 80° C. for 15 h. The reaction was cooled to rt and filtered through Celite and washed with EtOAc. The residue was purified by flash chromatography (ISCO, 24 g silica, 0-40% EtOAc/Hex) provided tert-butyl 3-(5-((diphenylmethylene)amino)pyridin-3-yl) morpholine-4-carboxylate as a light yellow solid (236 mg, 49%). A solution of tert-butyl 3-(5-((diphenylmethylene)amino)pyridin-3-yl) morpholine-4-carboxylate (184 mg, 0.415 mmol) in MeOH (6.9 mL) was cooled to 0° C. and hydroxylamine hydrochloride (86 mg, 1.245 mmol) and sodium acetate (170 mg, 2.0 mmol) were added sequentially. The reaction was removed from ice and stirred at rt for 2 h. The reaction mixture was poured into aqueous NH₄Cl and extracted with EtOAc (3×50 mL). The combined organics were washed with brine, dried (Na₂SO₄) and concentrated in vacuo. The residue was purified by flash chromatography (ISCO, 24 g silica, 0-15% MeOH (w/5% NH₄OH) in DCM) provided the title compound as a light beige solid (90 mg, 78%). LCMS m/z=280 [M+H]⁺.

### Intermediate A69. rel-tert-butyl(2S,5S)-2-(3-aminophenyl)-5-methylpyrrolidine-carboxylate

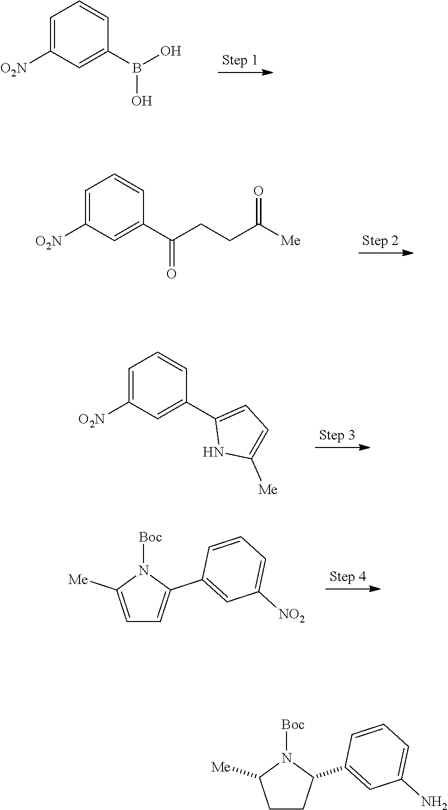 [SMILES: O=[N+]([O-])c1cccc(B(O)O)c1 ; CC(=O)CCC(=O)c1cccc([N+](=O)[O-])c1 ; Cc1ccc(-c2cccc([N+](=O)[O-])c2)[nH]1 ; Cc1ccc(-c2cccc([N+](=O)[O-])c2)n1C(=O)OC(C)(C)C ; C[C@H]1CC[C@@H](c2cccc(N)c2)N1C(=O)OC(C)(C)C]

### Step 1. Synthesis of 1-(3-nitrophenyl) pentane-1,4-dione

A solution of levulinic acid (1.749 g, 15.0 mmol), H₂O (0.70 mL, 37.5 mmol) and pivalic anhydride (4.6 mL, 22.5 mmol) in THF (40 mL) was added to Pd(OAc)₂ (109 mg, 0.45 mmol), tris(4-methoxyphenyl)phosphine (377 mg, 1.05 mmol), (3-nitrophenyl) boronic acid (3.061 g, 18.0 mmol) in THF (50 mL). The flask was purged vigorously with N₂ for 10 min, sealed and heated to 60° C. for 42 h. The mixture was filtered through Celite, washed with EtOAc (100 mL). The filtrate was evaporated to remove THF, diluted with EtOAc (150 mL), washed with sat aq NaHCO₃ (3×), brine, dried (MgSO₄) and evaporated to dryness. The residue was purified by flash chromatography (ISCO, silica, 0-10% MeOH/DCM) to afford a residue that was dissolved in EtOAc (50 mL) and washed with (3×, 1M NaOH), H₂O (2×), brine, dried (MgSO₄) and concentrated by rotary evaporation to provide the title compound as a yellow solid (473 mg, 14%). LCMS m/z=222 [M+H]⁺.

### Step 2. Synthesis of 2-methyl-5-(3-nitrophenyl)-1H-pyrrole

MgSO₄ (511 mg, 4.25 mmol, 2.1 equiv) was added to a solution of 1-(3-nitrophenyl) pentane-1,4-dione (Step 1, 461 mg, 2.05 mmol) and ammonium acetate (483 mg, 6.15 mmol) in MeOH (40 mL). Glacial acetic acid (0.12 mL, 2.05 mmol) was added with stirring at rt and the mixture stirred at rt for 4 days. The reaction mixture was evaporated to dryness in vacuo and the residue dissolved in EtOAc (50 mL), washed with water (2×), saturated aqueous sodium bicarbonate, brine, dried (MgSO₄) and evaporated to dryness in vacuo. The residue was purified by flash chromatography (ISCO 24 g silica, 0-10% MeOH/DCM) to afford the title compound as a red-orange solid (298 mg, 72%). LCMS m/z=203 [M+H]⁺.

### Step 3. Synthesis of tert-butyl 2-methyl-5-(3-nitrophenyl)-1H-pyrrole-1-carboxylate

To a solution of 2-methyl-5-(3-nitrophenyl)-1H-pyrrole (Step 2, 376 mg, 1.85 mmol) in MeCN (8 mL) were added DMAP (46.4 mg, 0.36 mmol) and Boc₂O (786 mg, 3.60 mmol) and the mixture heated to 80° C. for 22 h. The mixture was poured into dilute aqueous HCl and extracted with EtOAc (3×50 mL). The combined organics were washed with sat aq NaHCO₃, water (2×), brine, dried (MgSO₄) and evaporated to dryness in vacuo. The residue was purified by flash chromatography (ISCO, 24 g silica, 0-75% EtOAc/hex) provided the title compound as a red-orange solid (434 mg, 80%). LCMS m/z=303 [M+H]⁺.

### Step 4. Synthesis of rel-tert-butyl(2S,5S)-2-(3-aminophenyl)-5-methylpyrrolidine-1-carboxylate

To a solution of tert-butyl 2-methyl-5-(3-nitrophenyl)-1H-pyrrole-1-carboxylate (Step 3, 458 mg, 1.50 mmol) in MeOH (15 mL) was added Pt/C (5 wt %, 616 mg, 0.15 mmol) and the reaction flask sparged with H₂ for 10 min and stirred at rt overnight under a balloon atmosphere of H₂. The reaction was filtered through Celite and washed with MeOH. The combined organics were evaporated to dryness and the residue purified using 2 rounds of flash chromatography (ISCO, 24 g silica, 0-80% EtOAc/hex; followed by ISCO, 12 g silica, 20-90% EtOAc/hex) to afford the title compound as a highly viscous colourless oil (293 mg, 70%). LCMS m/z=277 [M+H]⁺.

### Intermediate A70. tert-butyl(1-(3-aminophenyl)cyclobutyl)carbamate

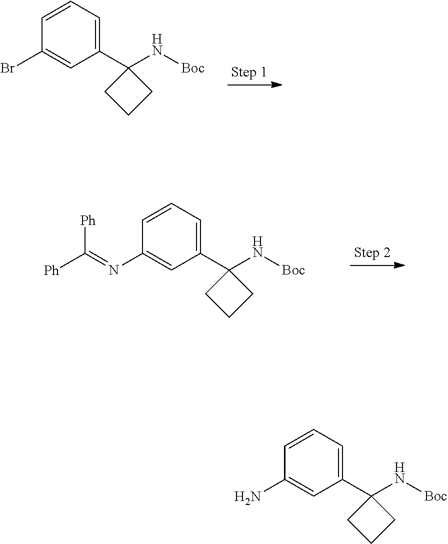 [SMILES: CC(C)(C)OC(=O)NC1(c2cccc(Br)c2)CCC1 ; CC(C)(C)OC(=O)NC1(c2cccc(N=C(c3ccccc3)c3ccccc3)c2)CCC1 ; CC(C)(C)OC(=O)NC1(c2cccc(N)c2)CCC1]

### Step 1. Synthesis of tert-butyl(1-(3-((diphenylmethylene)amino)phenyl)cyclobutyl)carbamate

To a solution of tert-butyl(1-(3-bromophenyl)cyclobutyl)carbamate (500 mg, 1.53 mmol) and diphenylmethanimine (306 mg, 1.69 mmol) in dioxane (10 mL) was added Pd₂ (dba)₃ (140 mg, 0.153 mmol, 0.1 eq), Xantphos (177 mg, 0.306 mmol) and Cs₂CO₃ (1.50 g, 4.60 mmol) and the mixture stirred at 100° C. for 3 h under N₂. The reaction mixture was filtered and the filtrate concentrated under reduced pressure and the residue purified by MPLC (SiO₂, 20% EtOAc/PE) to give the title compound as a green oil (700 mg, crude). LCMS m/z=427 [M+H]⁺.

### Step 2. Synthesis of tert-butyl(1-(3-aminophenyl)cyclobutyl)carbamate

To a solution of tert-butyl(1-(3-((diphenylmethylene)amino)phenyl)cyclobutyl)carbamate (Step 1, 650 mg, 1.52 mmol) in MeOH (10 mL) was added hydroxylamine hydrochloride (2.54 g, 36.6 mmol) and NaOAc (5.63 g, 68.6 mmol) and the mixture stirred at 25° C. for 2 h.

The reaction mixture was added to saturated aqueous NaHCO₃ solution (10 mL) and concentrated under reduced pressure to remove solvent. The residue was extracted with EtOAc (3×50 mL) and the combined organics dried (Na₂SO₄) and concentrated under reduced pressure and the residue purified by MPLC (SiO₂, 50% EtOAc/PE) to give the title compound as a yellow solid (330 mg, 82%). LCMS m/z=207 [M+H]⁺.

### Intermediate A71. 2-(((5-aminopyridin-3-yl)methyl)amino) ethan-1-ol

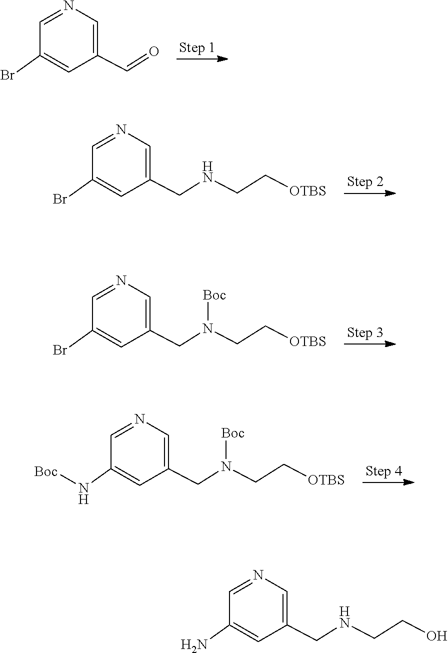 [SMILES: O=Cc1cncc(Br)c1 ; CC(C)(C)[Si](C)(C)OCCNCc1cncc(Br)c1 ; CC(C)(C)OC(=O)N(CCO[Si](C)(C)C(C)(C)C)Cc1cncc(Br)c1 ; CC(C)(C)OC(=O)Nc1cncc(CN(CCO[Si](C)(C)C(C)(C)C)C(=O)OC(C)(C)C)c1 ; Nc1cncc(CNCCO)c1]

### Step 1. Synthesis of N-((5-bromopyridin-3-yl)methyl)-2-((tert-butyldimethylsilyl)oxy) ethan-1-amine

To a solution of 5-bromopyridine-3-carbaldehyde (5 g, 26.9 mmol) in EtOH (20 mL) was added 2-((tert-butyldimethylsilyl)oxy) ethan-1-amine (5.18 g, 29.6 mmol) at 25° C. and the mixture stirred at 25° C. for 12 h under N₂. To this was added NaBH₄ (2.03 g, 53.8 mmol) was added at 0° C. and the resulting mixture stirred at 25° C. for 2 h under N₂. The reaction mixture was quenched by addition saturated NH₄Cl (25 mL) at 0° C. and extracted with EtOAc (3×20 mL). The combined organics were dried (Na₂SO₄) and concentrated under reduced pressure to give the title compound as a yellow solid (8.7 g, 94%). LCMS m/z=347 [M+H]⁺.

### Step 2. Synthesis of tert-butyl ((5-bromopyridin-3-yl)methyl) (2-((tert-butyldimethylsilyl)oxy)ethyl)carbamate

(Boc)₂O (5.37 g, 24.61 mmol) was added to mixture of N-((5-bromopyridin-3-yl)methyl)-2-((tert-butyldimethylsilyl)oxy) ethan-1-amine (Step 1, 8.5 g, 24.6 mmol), Na₂CO₃ (5.22 g, 49.2 mmol) in THF (30 mL) and H₂O (10 mL) and the mixture stirred at 25° C. for 1 h. The reaction mixture was filtered and the filtrate diluted with H₂O (30 mL) and extracted with EtOAc (3×30 mL). The combined organics were washed with brine 40 mL (2×20 mL), dried (Na₂SO₄) and concentrated under reduced pressure and the residue was purified by column chromatography (SiO₂, 0-100% EtOAc/PE) to give the title compound as a yellow solid (9.5 g, 86%). LCMS m/z=447 [M+H]⁺.

### Step 3. Synthesis of tert-butyl ((5-((tert-butoxycarbonyl)amino)pyridin-3-yl)methyl) (2-((tert-butyldimethylsilyl)oxy)ethyl)carbamate

A mixture of tert-butyl ((5-bromopyridin-3-yl)methyl) (2-((tert-butyldimethylsilyl)oxy)ethyl)carbamate (Step 2, 9.3 g, 20.9 mmol), (Boc)₂₀ (4.89 g, 41.8 mmol), Pd₂ (dba)₃ (1.91 g, 2.09 mmol), XPhos (995 mg, 2.09 mmol) and Cs₂CO₃ (10.20 g, 31.32 mmol) in dioxane (80 mL) was degassed and purged with N₂ (3×) and stirred at 100° C. for 2 h under N₂. The reaction mixture was filtered and the filtrate diluted with H₂O (60 mL) and extracted with EtOAc (3×50 mL). The combined organics were washed with brine (2×30 mL), dried (Na₂SO₄), concentrated under reduced pressure and the residue was purified by column chromatography (SiO₂, 0-100% EtOAc/PE) to give the title compound as a yellow solid (9.5 g, 94%). LCMS m/z=482 [M+H]⁺.

### Step 4. Synthesis of 2-(((5-aminopyridin-3-yl)methyl)amino) ethan-1-ol

A mixture of tert-butyl ((5-((tert-butoxycarbonyl)amino)pyridin-3-yl)methyl) (2-((tert-butyldimethylsilyl)oxy)ethyl)carbamate (Step 3, 1.5 g, 3.11 mmol) in HCl/EtOAc (20 mL) was stirred at 25° C. for 12 h. The reaction mixture was concentrated under N₂ and the residue diluted with EtOAc (30 mL) and the pH adjusted to pH>7 with NH₄OH at 0° C. The mixture was filtered and the filtrate concentrated under reduced pressure. The residue was purified by prep-HPLC-11 (1-15% MeCN) to give the title compound as a yellow solid (150 mg, 29%). LCMS m/z=168 [M+H]⁺.

### Intermediate A72. rel-tert-butyl(2S,5S)-2-(3-aminophenyl)-5-methylpyrrolidine-1-carboxylate

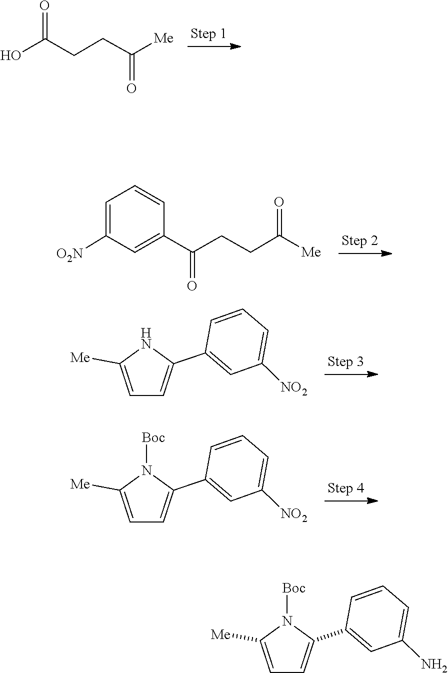

### Step 1. Synthesis of 1-(3-nitrophenyl) pentane-1,4-dione

To a mixture of Pd(OAc)₂ (109 mg, 0.45 mmol), tris(4-methoxyphenyl)phosphine (377 mg, 1.05 mmol) and (3-nitrophenyl) boronic acid (3.061 g, 18.0 mmol) in THF (50 mL) was added a solution of levulinic acid (1.749 g, 15.0 mmol), water (0.70 mL, 37.5 mmol, 2.5 equiv) and pivalic anhydride (4.6 mL, 22.5 mmol) in THF (40 mL) and the mixture purged vigorously with N₂ for 10 min and then sealed and heated to 60° C. for 42 h. The mixture was filtered through Celite, washed with EtOAc (100 mL). The filtrate was concentrated by rotary evaporation diluted with EtOAc (150 mL) and washed with sat aq sodium bicarbonate (3×), brine, dried (MgSO₄) and evaporated to dryness. The residue was purified by flash chromatography (ISCO, 80 silica, 0-10% MeOH/DCM) to afford a mixture of the title compound and pivalic acid. The mixed fractions were dissolved in EtOAc (50 mL) and washed three times with 1 M aqueous sodium hydroxide, twice with water, and once with brine, dried over MgSO₄, filtered, and concentrated by rotary evaporation to provide the title compound as a yellow solid (473 mg, 14%). LCMS m/z=221 [M+H].

### Step 2. Synthesis of 2-methyl-5-(3-nitrophenyl)-1H-pyrrole

MgSO₄ (511 mg, 4.25 mmol) was added to a solution of 1-(3-nitrophenyl) pentane-1,4-dione (Step 1, 461 mg, 2.05 mmol) and ammonium acetate (483 mg, 6.15 mmol) in MeOH (40 mL) and whilst stirring at rt was added glacial acetic acid (0.12 mL, 2.05 mmol) and the mixture stirred at rt for 4 days. The reaction mixture was evaporated to dryness and the residue dissolved in EtOAc (50 mL), washed with H₂O (×2), sat aq sodium bicarbonate, brine, dried (MgSO₄) evaporated to dryness in vacuo. The residue was purified by flash chromatography (ISCO 24 g silica, 0-0% MeOH/DCM) provided the title compound as a red-orange solid (298 mg, 72%). LCMS m/z=203 [M+H]⁺.

### Step 3. tert-butyl 2-methyl-5-(3-nitrophenyl)-1H-pyrrole-1-carboxylate

To a solution of 2-methyl-5-(3-nitrophenyl)-1H-pyrrole (Step 2, 376 mg, 1.85 mmol) in MeCN (8.0 mL) were added DMAP (46.4 mg, 0.36 mmol) and Boc₂O (786 mg, 3.60 mmol) and the reaction mixture heated to 80° C. for 22 h. The reaction mixture was poured into dilute aqueous HCl and extracted with EtOAc (3×50 mL). The combined organics were washed with sat aq sodium bicarbonate, H₂O (2×), brine, dried (MgSO₄) and evaporated to dryness in vacuo. The residue was purified by flash chromatography (ISCO, 24 g silica, 0-75% EtO Ac/hex) to afford the title compound as a red-orange solid (434 mg, 80%). LCMS m/z=303 [M+H]⁺.

### Step 4. rel-tert-butyl(2S,5S)-2-(3-aminophenyl)-5-methylpyrrolidine-1-carboxylate

To a solution of tert-butyl 2-methyl-5-(3-nitrophenyl)-1H-pyrrole-1-carboxylate (Step 4, 458 mg, 1.50 mmol) in MeOH (15 mL) was added Pt/C (5 wt %, 616 mg, 0.15 mmol) and the reaction flask purged with H₂ for 10 min and then stirred at rt overnight with a balloon atmosphere of H₂. The reaction mixture was filtered through Celite, washed with MeOH and the filtrate evaporated to dryness. The residue was purified by two rounds of flash chromatography (ISCO, 24 g silica, 0-80% EtOAc/hex, followed by ISCO, 12 g silica, 20-90% EtO Ac/hex) to afford the title compound as a clear, colorless, highly viscous oil (293 mg, 70%). NMR analysis was unable to confirm the relative stereochemistry due to signal broadening; however, the relative stereochemistry is presumed to be cis-given literature precedent. LCMS m/z=277 [M+H]⁺.

### Intermediate A73. 5-(((2-methoxyethyl)amino)methyl)pyridin-3-amine

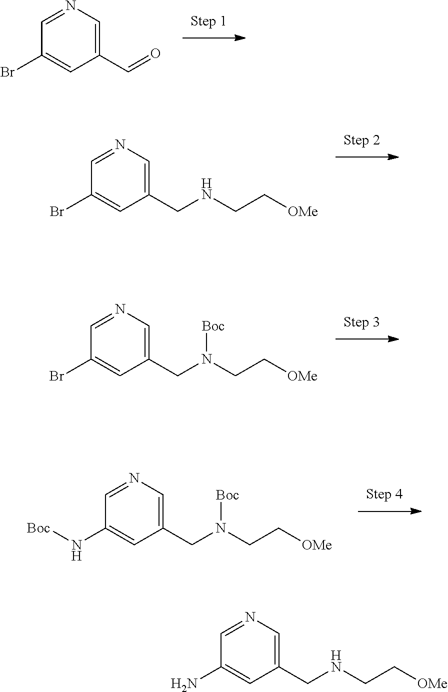 [SMILES: O=Cc1cncc(Br)c1 ; COCCNCc1cncc(Br)c1 ; COCCN(Cc1cncc(Br)c1)C(=O)OC(C)(C)C ; COCCN(Cc1cncc(NC(=O)OC(C)(C)C)c1)C(=O)OC(C)(C)C ; COCCNCc1cncc(N)c1]

The title compound was prepared using an analogous 4-Step method as described for Intermediate A71. LCMS m/z=182 [M+H]⁺.

### Intermediate A74. tert-butyl(R)-2-(5-aminopyridin-3-yl)-5-oxopiperazine-1-carboxylate or tert-butyl(S)-2-(5-aminopyridin-3-yl)-5-oxopiperazine-1-carboxylate

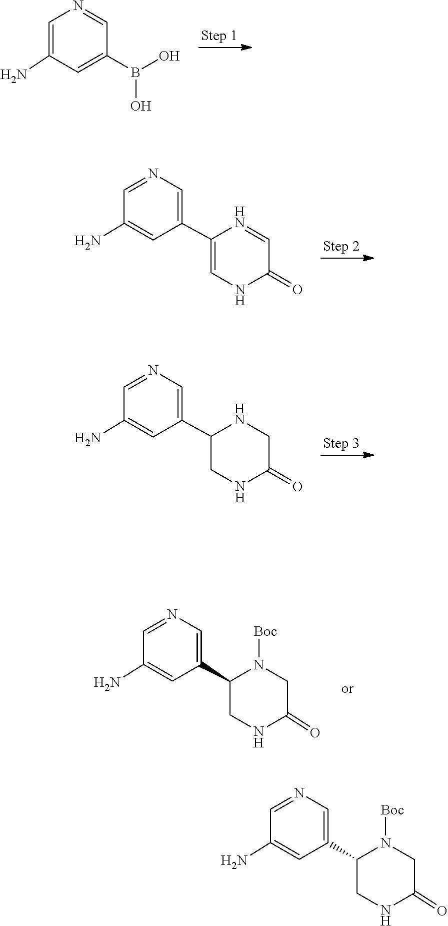 [SMILES: Nc1cncc(B(O)O)c1 ; Nc1cncc(-c2c[nH]c(=O)cn2)c1 ; Nc1cncc(C2CNC(=O)CN2)c1 ; CC(C)(C)OC(=O)N1CC(=O)NC[C@H]1c1cncc(N)c1 ; CC(C)(C)OC(=O)N1CC(=O)NC[C@@H]1c1cncc(N)c1]

### Step 1. Synthesis of 5-(5-aminopyridin-3-yl) pyrazin-2 (1H)-one

To a solution of (5-amino-3-pyridyl) boronic acid (78.83 mg, 0.57 mmmol) and 5-bromopyrazin-2-ol (50 mg, 0.285 mmol) in H₂O (0.5 mL) and EtOH (2 mL) was added K₃PO₄ (121.31 mg, 0.57 mmol) and cataCXium® A Pd G2 (19.11 mg, 0.028 mmol) and the mixture stirred at 80° C. for 1 hr under N₂. The reaction mixture was filtered and the filtrate was concentrated under reduced pressure to give the title compound as a white solid (2.1 g, 39%). LCMS m/z=189 [M+H]⁺.

### Step 2. Synthesis of 5-(5-aminopyridin-3-yl) piperazin-2-one

To a solution of 5-(5-amino-3-pyridyl)-1H-pyrazin-2-one (Step 2, 1.9 g, 10.10 mmol) in MeOH (10 mL) and DMF (30 mL) was added Pd/C (1.9 g, 10.10 mmol, 10% purity) under N₂ and the suspension degassed under vacuum and purged with H₂ several times. The mixture was stirred under H₂ (50 psi) at 25° C. for 12 h. The reaction mixture was filtered and the filtrate was concentrated under reduced pressure to give the title compound as a yellow solid (3 g, crude). LCMS m/z=193 [M+H]⁺.

### Step 3. Synthesis of tert-butyl(R)-2-(5-aminopyridin-3-yl)-5-oxopiperazine-1-carboxylate or tert-butyl(S)-2-(5-aminopyridin-3-yl)-5-oxopiperazine-1-carboxylate

A solution of 5-(5-amino-3-pyridyl) piperazin-2-one (Step 2, 2 g, 10.40 mmol) and (Boc)₂O (2.27 g, 10.40 mmol) in MeOH (10 mL) was stirred at 25° C. for 1 h. The reaction mixture was filtered and the filtrate was concentrated under reduced pressure 5-(5-amino-3-pyridyl) piperazin-2-one as yellow solid. The residue was separated by SFC (DAICEL CHIRALPAK IG, 250×30 mm, 10 μm); 50% MeOH (0.1% NH₄OH) in CO₂) to afford:

Peak 1, Intermediate A74. tert-butyl(R)-2-(5-aminopyridin-3-yl)-5-oxopiperazine-1-carboxylate or tert-butyl(S)-2-(5-aminopyridin-3-yl)-5-oxopiperazine-1-carboxylate (white solid; 75 mg, 2.5%). LCMS m/z=293 [M+H]⁺.

### Intermediate A75. Synthesis of tert-butyl(S)-2-(3-amino-2-morpholinophenyl) pyrrolidine-1-carboxylate and tert-butyl(R)-2-(3-amino-2-morpholinophenyl) pyrrolidine-1-carboxylate

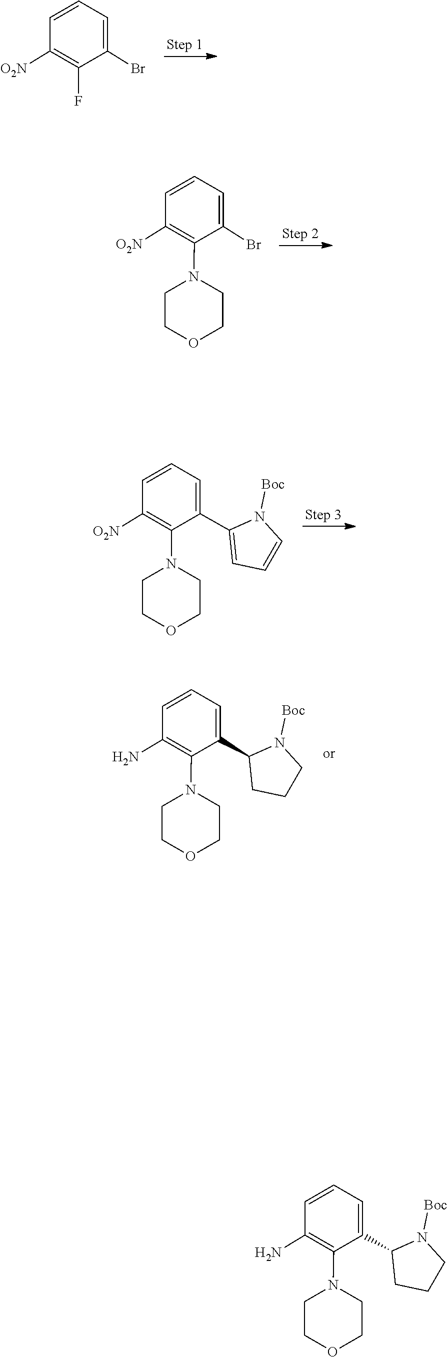 [SMILES: O=[N+]([O-])c1cccc(Br)c1F ; O=[N+]([O-])c1cccc(Br)c1N1CCOCC1 ; CC(C)(C)OC(=O)n1cccc1-c1cccc([N+](=O)[O-])c1N1CCOCC1 ; CC(C)(C)OC(=O)N1CCC[C@H]1c1cccc(N)c1N1CCOCC1 ; CC(C)(C)OC(=O)N1CCC[C@@H]1c1cccc(N)c1N1CCOCC1]

### Step 1. Synthesis of 4-(2-bromo-6-nitrophenyl) morpholine

To a solution of 1-bromo-2-fluoro-3-nitro-benzene (2 g, 9.09 mmol) and morpholine (792 mg, 9.09 mmol) in DMSO (5 mL) was added DIPEA (3.52 g, 27.3 mmol) at 25° C. and the reaction mixture stirred at 100° C. for 16 h. Water (5 ml) was added and the reaction mixture filtered and filtrate concentrated under reduced pressure to give the title compound as a yellow solid (2.5 g, crude). ¹H NMR (400 MHz, CDCl₃) δ: 7.79 (d, 1H), 7.57 (d, 1H), 7.10 (t, 1H), 3.90-3.77 (m, 4H), 3.21-3.07 (m, 4H).

### Step 2. Synthesis of tert-butyl 2-(2-morpholino-3-nitrophenyl)-1H-pyrrole-1-carboxylate

To a solution of 4-(2-bromo-6-nitro-phenyl) morpholine (Step 11, 2.40 g, 8.36 mmol) and tert-butyl 2-(4,4,5,5-tetramethyl-1,3,2-dioxaborolan-2-yl) pyrrole-1-carboxylate (5.88 g, 20.1 mmol) in DME (35 mL) and H₂O (7 mL) was added Na₂CO₃ (1.77 g, 16.7 mmol) and Pd(PPh₃)₄ (1.45 g, 0.418 mmol) at 25° C. and the reaction mixture stirred at 90° C. for 2 h under N₂. The reaction mixture was filtered and the filtrate concentrated under reduced pressure to give a residue. The residue was purified by MPLC (SiO₂, 10% EtOAc/PE) to give the title compound as a yellow solid (2.5 g, 80%). LCMS m/z=374 [M+H]⁺.

### Step 3. Synthesis of tert-butyl 2-(3-amino-2-morpholinophenyl) pyrrolidine-1-carboxylate

Pd/C (1.50 g, 10% purity) was added to a mixture of tert-butyl 2-(2-morpholino-3-nitrophenyl)-1H-pyrrole-1-carboxylate (2.5 g, 6.70 mmol) in THF (20 mL) at 25° C. and the resulting mixture stirred at 50° C. for 16 h under H₂ (15 psi). The reaction mixture was filtered and the filtrate was concentrated under reduced pressure and the residue purified by prep-HPLC-17 (25-60% MeCN). The residue was further separated by SFC (Chiralpak IC, 250×30 mm, 10 μm; 40% IPA (0.1% NH₄OH) in CO₂) to afford:

Peak 2, Intermediate A75, tert-butyl(S)-2-(3-amino-2-morpholinophenyl) pyrrolidine-1-carboxylate or tert-butyl(R)-2-(3-amino-2-morpholinophenyl) pyrrolidine-1-carboxylate as a white solid (0.2 g, 9%). LCMS m/z=348 [M+H]⁺.

### Intermediate A76 and A77. tert-butyl(S)-2-(3-amino-2-morpholinophenyl) pyrrolidine-1-carboxylate or tert-butyl(R)-2-(3-amino-2-morpholinophenyl) pyrrolidine-1-carboxylate

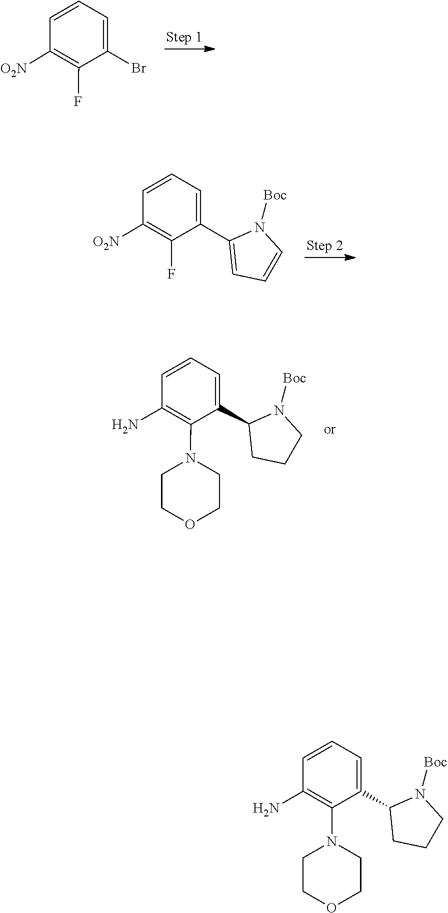 [SMILES: O=[N+]([O-])c1cccc(Br)c1F ; CC(C)(C)OC(=O)n1cccc1-c1cccc([N+](=O)[O-])c1F ; CC(C)(C)OC(=O)N1CCC[C@H]1c1cccc(N)c1N1CCOCC1 ; CC(C)(C)OC(=O)N1CCC[C@@H]1c1cccc(N)c1N1CCOCC1]

### Step 1. Synthesis of tert-butyl 2-(2-fluoro-3-nitrophenyl)-1H-pyrrole-1-carboxylate

The title compound was prepared as a yellow oil (2.5 g, 90%) from 1-bromo-2-fluoro-3-nitro-benzene and tert-butyl 2-(4,4,5,5-tetramethyl-1,3,2-dioxaborolan-2-yl) pyrrole-1-carboxylate using an analogous method to that described for Intermediate A75, Step 2). ¹H NMR (400 MHz, CDCl₃) δ: 8.07-7.96 (m, 1H), 7.69-7.62 (m, 1H), 7.55-7.47 (m, 1H), 7.35-7.30 (m, 1H), 6.36-6.29 (m, 2H), 1.44 (s, 9H).

### Step 2. Synthesis of tert-butyl(S)-2-(3-amino-2-morpholinophenyl) pyrrolidine-1-carboxylate or tert-butyl(R)-2-(3-amino-2-morpholinophenyl) pyrrolidine-1-carboxylate

The title compound was prepared from tert-butyl 2-(2-fluoro-3-nitrophenyl)-1H-pyrrole-1-carboxylate (Step 1) using an analogous method to that described for A75, Step 3. Chiral-SFC (DAICEL CHIRALCEL OJ, 250×30 mm, 10 μm); 25% IPA (0.1% NH₄OH) in CO₂).

Peak 1, Intermediate A76, tert-butyl(S)-2-(3-amino-2-morpholinophenyl) pyrrolidine-1-carboxylate or tert-butyl(R)-2-(3-amino-2-morpholinophenyl) pyrrolidine-1-carboxylate (White solid: 20 mg, 11%). LCMS m/z=225 [M−56+H]⁺.

Peak 2, Intermediate A77, tert-butyl(R)-2-(3-amino-2-morpholinophenyl) pyrrolidine-1-carboxylate or tert-butyl(S)-2-(3-amino-2-morpholinophenyl) pyrrolidine-1-carboxylate (White solid: 20 mg, 11%). LCMS m/z=225 [M−56+H]⁺.

### Intermediate A78. tert-butyl(3-amino-2-(trifluoromethyl)benzyl)carbamate

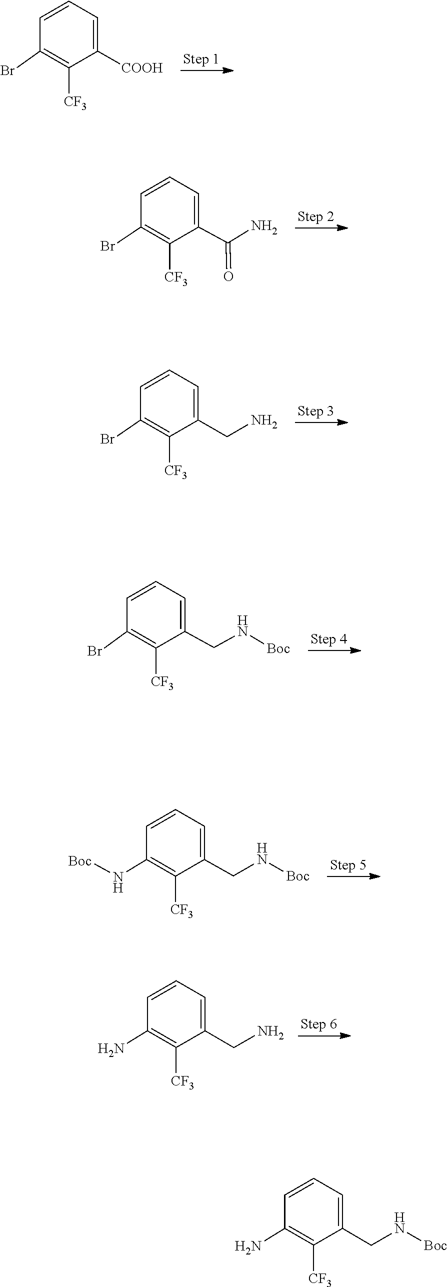

### Step 1. Synthesis of 3-bromo-2-(trifluoromethyl)benzamide

To a solution of 3-bromo-2-(trifluoromethyl)benzoic acid (2.5 g, 9.29 mmol) in DMF (15 mL) was added NH₄Cl (2.49 g, 46.5 mmol), HATU (5.30 g, 13.9 mmol) and DIPEA (7.21 g, 55.8 mmol) and the mixture stirred at 25° C. for 2 h. Water (50 mL) was added the mixture extracted with EtOAc (3×30 mL). The combined organics were dried (Na₂SO₄), concentrated under reduced pressure and the residue purified by prep-HPLC-21 (15-45% MeCN) to give the title compound as a white solid (3.50 g, 70%). ¹H NMR (400 MHz, DMSO-d₅) δ: 8.01 (s, 1H), 7.92 (d, 1H), 7.67 (s, 1H), 7.60 (t, 1H), 7.46 (d, 1H).

### Step 2. Synthesis of (3-bromo-2-(trifluoromethyl)phenyl) methanamine

To a mixture of 3-bromo-2-(trifluoromethyl)benzamide (Step 1, 1.50 g, 5.60 mmol) in THF (25 mL) was added BH₃. THF (1 M, 84 mL) at 0° C. and the mixture stirred at 50° C. for 17 h under N₂. The reaction mixture was quenched by addition of MeOH (20 mL) at 0° C. and the reaction mixture concentrated under reduced pressure. The residue was purified by prep-HPLC-22 (5-35% MeCN) to give the title compound as a white solid (950 mg, 33%). LCMS m/z=254 [M+H]⁺.

### Step 3. Synthesis of tert-butyl(3-bromo-2-(trifluoromethyl)benzyl)carbamate

To a solution of (3-bromo-2-(trifluoromethyl)phenyl) methanamine (Step 2, 830 mg, 3.27 mmol), (Boc)₂₀ (1.07 g, 4.90 mmol) in H₂O (2 mL) and THF (10 mL) was added Na₂CO₃ (693 mg, 6.53 mmol) and the mixture stirred at 25° C. for 1 h. The mixture was filtered and the filtrate concentrated under reduced pressure and the residue purified by prep-TLC (25% EtOAc/PE) to give the title compound as a yellow oil (460 mg, 40%). LCMS m/z=298 [M−56+H]⁺.

### Step 4. Synthesis of tert-butyl(3-((tert-butoxycarbonyl)amino)-2-(trifluoromethyl)benzyl)carbamate

To a solution of tert-butyl(3-bromo-2-(trifluoromethyl)benzyl)carbamate (Step 3, 410 mg, 1.16 mmol) and tert-butyl carbamate (203 mg, 1.74 mmol) in dioxane (10 mL) was added Cs₂CO₃ (566 mg), Xphos (55.2 mg, 0.116 mmol) and Pd₂ (dba)₃ (106 mg, 0.116 mmol) and the mixture stirred at 100° C. for 2 h under N₂. The mixture was concentrated under reduced pressure and the residue purified by prep-TLC (20% EtOAc/PE) to give the title compound as a yellow oil (370 mg, 82%). LCMS m/z=235 [M−156+H]⁺.

### Step 5. Synthesis of 3-(aminomethyl)-2-(trifluoromethyl) aniline

A solution of tert-butyl(3-((tert-butoxycarbonyl)amino)-2-(trifluoromethyl)benzyl)carbamate (Step 4, 360 mg, 0.922 mmol) in DCM (3 mL) and TFA (1 mL) was stirred at 25° C. for 0.5 h. The reaction mixture was filtered and the filtrate was concentrated under reduced pressure to give the title compound as a yellow solid (110 mg, 63%). LCMS m/z=191 [M+H]⁺.

### Step 6. Synthesis of tert-butyl(3-amino-2-(trifluoromethyl)benzyl)carbamate

To a solution of 3-(aminomethyl)-2-(trifluoromethyl) aniline (Step 5, 80 mg, 0.42 mmol) and (Boc)₂₀ (73.5 mg, 0.337 mmol) in H₂O (0.60 mL) and THF (3 mL) was added Na₂CO₃ (89.2 mg, 0.841 mmol) and the mixture stirred at 25° C. for 1 h. The reaction mixture was filtered, the filtrate concentrated under reduced pressure and the residue purified by prep-HPLC-11 (20-55% MeCN) to give the title compound as a yellow oil (40 mg, 33%). LCMS m/z=235 [M−56+H]⁺.

### Intermediate A79. tert-butyl(R)-2-(5-aminopyridazin-3-yl) piperidine-1-carboxylate or tert-butyl(S)-2-(5-aminopyridazin-3-yl) piperidine-1-carboxylate

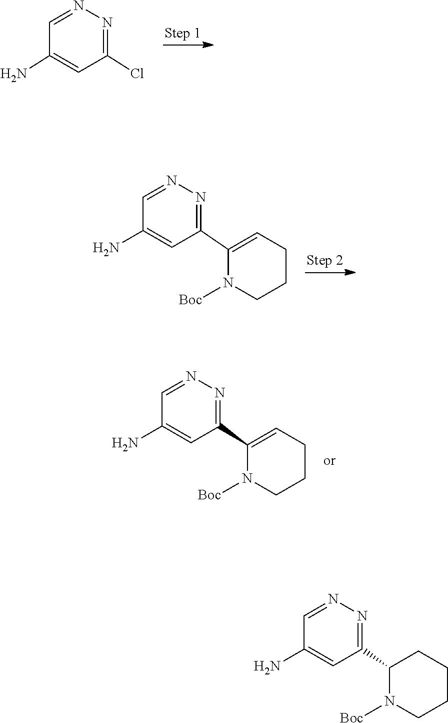

The title compounds were prepared from 6-chloropyridazin-4-amine and tert-butyl 6-(4,4,5,5-tetramethyl-1,3,2-dioxaborolan-2-yl)-3,4-dihydropyridine-1 (2H)-carboxylate using an analogous 2-step method as described for Intermediate A20. Chiral-SFC (Phenomenex Cellulose-2, 250×30 mm, 10 μm; 40% MeOH (0.1% NH₄OH) in CO₂.

Peak 1, Intermediate A79. tert-butyl(R)-2-(5-aminopyridazin-3-yl) piperidine-1-carboxylate or tert-butyl(S)-2-(5-aminopyridazin-3-yl) piperidine-1-carboxylate (white solid, 50 mg, 29%). LCMS m/z=279 [M+H]⁺.

### Intermediate A80. (R)-6-(1-aminoethyl) pyrazin-2-amine or (S)-6-(1-aminoethyl) pyrazin-2-amine

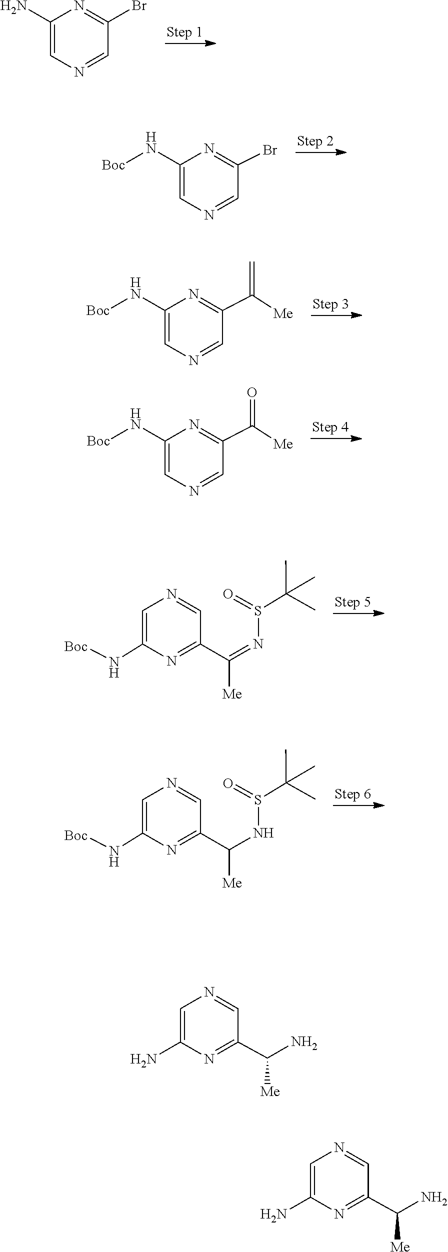

### Step 1. Synthesis of tert-butyl(6-bromopyrazin-2-yl)carbamate

To the solution of 6-bromopyrazin-2-amine (13 g, 74.7 mmol) in DCM (100 mL) was added DMAP (9.13 g, 74.7 mmol), TEA (11.34 g, 112 mmol) and Boc₂O (16.3 g, 74.7 mmol) at 0° C. and the mixture stirred at 25° C. for 2 h. The reaction mixture was concentrated under reduced pressure to give a residue that was purified by MPLC (SiO₂, 0-33% EtOAc/PE) to give the title compound as a yellow solid (15.31 g, 75%). LCMS m/z=274 [M+H]⁺.

### Step 2. Synthesis of tert-butyl(6-(prop-1-en-2-yl)pyrazin-2-yl)carbamate

To a solution tert-butyl(6-bromopyrazin-2-yl)carbamate (Step 1, 15.2 g, 55.5 mmol) and 4,4,5,5-tetramethyl-2-(prop-1-en-2-yl)-1,3,2-dioxaborolane (18.64 g, 111 mmol) in dioxane (100 mL) and H₂O (5 mL) was added K₂CO₃ (15.33 g, 111 mmol) and Pd(dppf) C₁₂ (1.22 g, 1.66 mmol) and the mixture stirred at 100° C. for 1 h under N₂. The reaction mixture was concentrated under reduced pressure and the residue purified by MPLC (SiO₂, 1-5% EtOAc/PE) to give the title compound as a yellow solid (12.1 g, 93%). LCMS m/z=236 [M+H]⁺.

### Step 3. Synthesis of tert-butyl(6-acetylpyrazin-2-yl)carbamate

Ozone was bubble through a solution of tert-butyl(6-(prop-1-en-2-yl) pyrazin-2-yl)carbamate (Step 2, 11 g, 46.8 mmol) in DCM (100 mL) at −78° C. until it turned light blue (1 h, 15 psi). The reaction was quenched with Me₂S (30.8 g, 496 mmol) at 25° C. and the resulting mixture stirred at 25° C. for 1 h. The mixture was diluted with H₂O (100 mL) and extracted with DCM (3×80 mL). The combined organics were dried (Na₂SO₄) and concentrated under reduced pressure. The residue was purified by MPLC (SiO₂, 1-17% EtOAc/PE) to give the title compound as a brown solid (3.13 g, 28%). LCMS m/z=238 [M+H]⁺.

### Step 4. Synthesis of tert-butyl(Z)-(6-(1-((tert-butylsulfinyl)imino) ethyl) pyrazin-2-yl)carbamate

To a solution of 2-methylpropane-2-sulfinamide (1.15 g, 9.48 mmol, 1.5 eq) and tert-butyl(6-acetylpyrazin-2-yl)carbamate (Step 3, 1.5 g, 6.32 mmol) in THF (20 mL) was added Ti(OEt)₄ (2.88 g, 12.6 mmol) and the mixture stirred at 60° C. for 12 h. The reaction mixture was diluted with H₂O (20 mL) and the solids removed by filtration. The filtrate was concentrated under reduced pressure and the residue diluted with H₂O (20 mL) and extracted with EtOAc (3×30 mL), dried (Na₂SO₄) and concentrated under reduced pressure to give the title compound as a brown oil (1.6 g, 74%). LCMS m/z=341 [M+H]⁺.

### Step 5. Synthesis of tert-butyl(6-(1-((tert-butylsulfinyl)amino)ethyl) pyrazin-2-yl)carbamate

To a solution of tert-butyl(Z)-(6-(1-((tert-butylsulfinyl)imino) ethyl) pyrazin-2-yl)carbamate (Step 4, 1.6 g, 4.70 mmol) in MeOH (20 mL) was added NaBH₄ (440 mg, 11.6 mmol) at 0° C. and the mixture stirred at 25° C. for 1 h. The reaction mixture was quenched by addition 0.5 M HCl (10 mL) at 0° C. and extracted with EtOAc (3×30 mL). The combined organics were dried (Na₂SO₄) and concentrated under reduced pressure and the residue purified by MPLC (SiO₂, 1-50% EtOAc/PE) to give the title compound as a brown solid (1.04 g, 65% yield). LCMS m/z=343 [M+H]⁺.

### Step 6. Synthesis of (R)-6-(1-aminoethyl) pyrazin-2-amine or (S)-6-(1-aminoethyl) pyrazin-2-amine

A solution of tert-butyl(6-(1-((tert-butylsulfinyl)amino)ethyl) pyrazin-2-yl)carbamate (Step 5, 1.04 g, 3.04 mmol) in HCl/MeOH (15 mL) was stirred at 25° C. for 0.5 h. The reaction mixture was concentrated under reduced pressure and the residue purified by prep-HPLC-11 (1-5% MeCN) followed by chiral SFC (DAICEL CHIRALPAK IC, 250×30 mm, 10 μm); 45% IPA (0.1% NH₄OH) in CO₂ to afford:

Peak 1, Intermediate A80. (R)-6-(1-aminoethyl) pyrazin-2-amine or (S)-6-(1-aminoethyl) pyrazin-2-amine (brown solid; 97 mg, 23%). LCMS m/z=139 [M+H]⁺.

### Intermediate A81. tert-butyl 2-(3-amino-2-methoxyphenyl) pyrrolidine-1-carboxylate

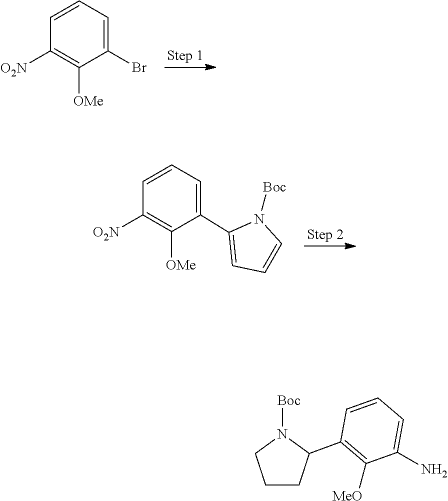 [SMILES: COc1c(Br)cccc1[N+](=O)[O-] ; COc1c(-c2cccn2C(=O)OC(C)(C)C)cccc1[N+](=O)[O-] ; COc1c(N)cccc1C1CCCN1C(=O)OC(C)(C)C]

### Step 1. Synthesis of tert-butyl 2-(2-methoxy-3-nitrophenyl)-1H-pyrrole-1-carboxylate

A mixture of 1-bromo-2-methoxy-3-nitrobenzene (463 mg, 2.00 mmol), (1-(tert-butoxycarbonyl)-1H-pyrrol-2-yl) boronic acid (633 mg, 3.00 mmol), tBuXPhos (63.7 mg, 0.15 mmol), and PdCl₂ (PPh₃)₂ (70.2 mg, 0.10 mmol) in dioxane (16 mL) and saturated aqueous sodium carbonate (4 mL) was sparged with nitrogen for 10 min and then heated to 80° C. overnight. The reaction mixture was filtered through Celite, washing with 75 mL EtOAc and concentrated by rotary evaporation. The residue was purified by flash chromatography (ISCO 40 g silica, 0-70% EtOAc/hex) to afford the title compound as a clear yellow oil (633 mg, 85% yield). LCMS m/z=341 [M+Na]⁺.

### Step 2. Synthesis of tert-butyl 2-(3-amino-2-methoxyphenyl) pyrrolidine-1-carboxylate

A mixture of tert-butyl 2-(2-methoxy-3-nitrophenyl)-1H-pyrrole-1-carboxylate (Step 1, 626 mg, 2.0 mmol) and Pt/C (5 wt %, 680 mg, 0.20 mmol, 10 mol %) in methanol (20 mL) was sparged with hydrogen for 5 min and then stirred at RT under a balloon atmosphere of hydrogen for 3 days. The reaction mixture was filtered through Celite, washed with methanol and evaporated to dryness. The residue was purified by flash chromatography (ISCO 24g silica, 0-5% MeOH/DCM) followed by (ISCO 24 g silica, 0-60% EtOAc/hex) to afford the title compound as a white solid (356 mg, 58%). LCMS m/z=293 [M+H]⁺.

### Intermediate A82. Synthesis of (3-amino-2-methoxyphenyl) methanol

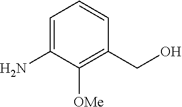 [SMILES: COc1c(N)cccc1CO]

A dry flask containing LiAlH₄ (484 mg, 12.5 mmol) was evacuated and backfilled three times with nitrogen and anhydrous THF (20 mL) added and the mixture cooled on ice. A suspension of 3-amino-2-methoxybenzoic acid (830 mg, 5.0 mmol) in anhydrous THF (30 mL) was added dropwise, with stirring over 15 min and the reaction warmed to RT and heated to 60° C. for 3 h. The reaction was cooled to RT and carefully quenched with dropwise addition of water. The mixture was diluted with EtOAc (10 mL), saturated aqueous NH₄Cl (1 mL) added and stirred at RT for 1.5 h. The mixture was poured into aqueous NaHCO₃ and extracted with EtOAc (4×50 mL). The combined organics were washed with water (×2), brine, dried (MgSO₄), and evaporated to dryness by rotary evaporation. The residue was purified by flash chromatography (ISCO 40 g silica, 0-20% MeOH/DCM) to give the title compound as a waxy amber-coloured solid (466 mg, 60%). LCMS m/z=154 [M+H]⁺.

### Intermediate A83. 3-(aminomethyl)-2-ethoxyaniline

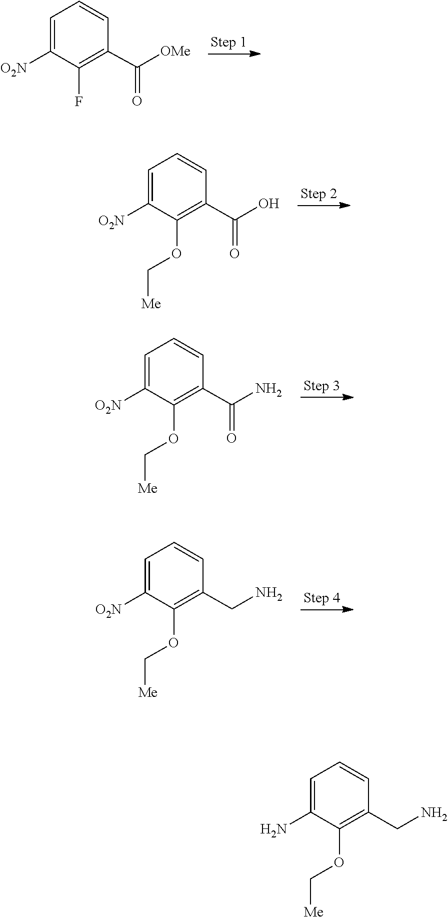

### Step 1. Synthesis of 2-ethoxy-3-nitrobenzoic acid

A solution of methyl 2-fluoro-3-nitrobenzoate (997 mg, 5.0 mmol) in DMF (12 mL) was flushed with N₂ and cooled on ice to this was added sodium ethoxide solution (2.00 mL, 21 w/w % in ethanol, 5.25 mmol) and the reaction stirred at 0° C. for 30 min and then at RT overnight. The reaction mixture was poured into water (100 mL) and extracted with EtOAc (3×30 mL). The combined organics were with water (×2), brine, dried (MgSO₄) and concentrated under reduced pressure. The residue was purified by chromatography (ISCO, 24 g silica, 0050% EtOAc/hex) and the residue (1.025 g) dissolved in MeOH (3 mL) and water (5 mL). To this was added NaOH (12.5 mmol, 497 mg) and the mixture heated to 60° C. overnight. The reaction mixture was acidified to pH 1 with aqueous HCl and the resulting precipitate collected by suction filtration. The solids were wash with water to give the title compound as a yellow solid (424 mg, 40% yield over 2 steps). LCMS m/z=212 [M+H]⁺.

### Step 2. Synthesis of 2-ethoxy-3-nitrobenzamide

To a solution of 2-ethoxy-3-nitrobenzoic acid (Step 1, 424 mg, 2.0 mmol) in MeCN (13.5 mL) added sequentially DIPEA (1.40 mL, 8.0 mmol), HOBT hydrate (459 mg, 3.0 mmol) and EDC-HCl (576 mg, 3.0 mmol) and the mixture stirred at RT for 10 min before ammonium carbonate (1.17 g, 18.0 mmol) was added and then stirred at RT for 3 days. The reaction was concentrated by rotary evaporation and water (30 mL) added to the residue. The resulting slurry was stirred at RT for 1 h and collected by suction filtration and washed with water (50 mL) to provide the title compound as a white solid (214 mg, 51%). LCMS m/z=211 [M+H]⁺.

### Step 3. Synthesis of (2-ethoxy-3-nitrophenyl) methanamine

A solution of 2-ethoxy-3-nitrobenzamide (Step 2, 211 mg, 1.0 mmol) in anhydrous THF (7 mL) was added dropwise to an ice-cold solution of BH₃-THF (2.30 mL, 1 M in THF, 2.30 mmol) and the reaction heated to 67° C. overnight. The reaction was cooled to RT, quenched with 6 M aqueous HCl (2 mL) and stirred for 1 hour. The volatiles were removed by rotary evaporation and the crude residue neutralized to pH >10 with saturated aqueous NaHCO₃, 1 M aqueous NaOH and extracted with EtOAc (4×25 mL). The combined organics were washed with water, brine, dried (MgSO₄) and concentrated by rotary evaporation. The residue was purified by chromatography (ISCO, 4 g silica, 0-10% MeOH/DCM) to provide the title compound as a yellow solid (39 mg, 20%). LCMS m/z=197 [M+H]⁺.

### Step 4. Synthesis of 3-(aminomethyl)-2-ethoxyaniline

A mixture of (2-ethoxy-3-nitrophenyl) methanamine (Step 3, 39.7 mg, 0.20 mmol) and Pd/C (19.1 mg, 10 wt %, 0.020 mmol) in MeOH (1.5 mL) was sparged with H₂ for 10 min and stirred at RT under a balloon atmosphere of H₂ for 3 h. The reaction was filtered through Celite, washed with MeOH and concentrated to provide the title compound as a yellow film (29.7 mg, 89%) which was used without purification. LCMS m/z=167 [M+H]⁺.

### Intermediate A84. tert-butyl(3-amino-2-(difluoromethoxy)benzyl)carbamate

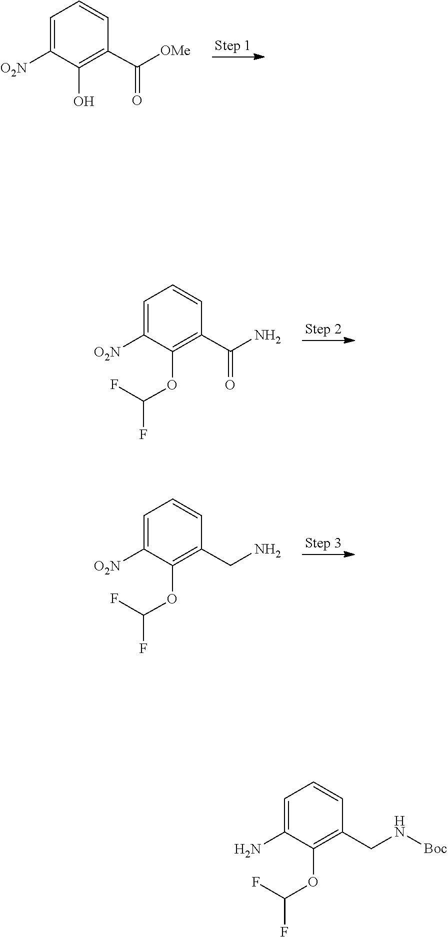 [SMILES: COC(=O)c1cccc([N+](=O)[O-])c1O ; NC(=O)c1cccc([N+](=O)[O-])c1OC(F)F ; NCc1cccc([N+](=O)[O-])c1OC(F)F ; CC(C)(C)OC(=O)NCc1cccc(N)c1OC(F)F]

### Step 1. Synthesis of 2-(difluoromethoxy)-3-nitrobenzamide

Part A: KOH (1.643 g, 30.0 mmol) and water (2.0 mL) to an ice-cold solution of methyl 2-hydroxy-3-nitrobenzoate (587 mg, 3.0 mmol) in MeCN (20 mL) and diethyl (bromodifluoromethyl)phosphonate (0.85 mL, 4.80 mmol) added dropwise over 2 min and the reaction stirred at 0° C. for 10 min and then at RT overnight. The reaction was poured into water and extracted with EtOAc (3×50 mL) and the organic component discarded. The aqueous phase was adjusted to pH<3 with aqueous HCl and extracted with EtOAc (3×50 mL). The combined organics were washed with water, brine, dried (MgSO₄) and concentrated under reduced pressure to afford a yellow solid (561 mg).

Part B: To a solution of 2-(difluoromethoxy)-3-nitrobenzoic acid (Part A, 561 mg, 2.4 mmol, ~35% component of mixture) in MeCN (16 mL) added sequentially DIPEA (1.60 mL, 9.60 mmol), HOBT hydrate (549 mg, 3.60 mmol) and EDC-HCl (686 mg, 3.60 mmol) and stirred at RT for 10 min and ammonium carbonate (1.40 g) added and the resulting mixture was stirred at RT for 3 days. The volatiles were removed by rotary evaporation and the residue poured into water and extracted with EtOAc (3×25 mL). The combined organics were washed with 1 M aqueous HCl (×3), saturated aqueous NaHCO₃, water, brine, dried over MgSO₄ and evaporated to dryness. The residue was purified by chromatography (ISCO 24g silica, 0-10% MeOH/DCM) to afford the title compound as a yellow solid (135 mg, 19% yield over 2 steps). LCMS m/z=233 [M+H]⁺.

### Step 2. Synthesis of (2-(difluoromethoxy)-3-nitrophenyl) methanamine

The reaction was run under a nitrogen atmosphere. BH₃-THF (2.40 mL, 1 M in THF, 2.40 mmol, 2.3 equiv) was cooled on ice. A solution of 2-(difluoromethoxy)-3-nitrobenzamide BJG-01-154 (239 mg, 1.02 mmol, 1.0 equiv) in anhydrous THF (7 mL) was added dropwise over 5 minutes. The reaction was heated to 67° C. overnight. The reaction was cooled to room temperature, quenched with 6 M aqueous HCl (3 mL), and stirred for 1 hour. Volatiles were removed by rotary evaporation. The crude residue was neutralized to pH >10 with 1 M aqueous NaOH, then extracted with EtOAc (4×30 mL). The combined organics were washed with water and brine, dried over MgSO₄, filtered, and concentrated by rotary evaporation. Flash chromatography (ISCO, 12 g silica, 0 to 10% MeOH/DCM, 10 min gradient) provided the title compound as a yellow-orange solid (65.6 mg, 29% yield). LCMS m/z=219 [M+H]⁺.

### Step 3. Synthesis of tert-butyl(3-amino-2-(difluoromethoxy)benzyl)carbamate

To a solution of (2-(difluoromethoxy)-3-nitrophenyl) methanamine (Step 2, 65.6 mg, 0.30 mmol) in DCM (2 mL) was added Boc₂O (73.5 mg, 0.30 mmol) followed by Et₃N (46 μL, 0.33 mmol) and the reaction stirred at RT for 4 h. The reaction was diluted with DCM (20 mL), washed with 1 M aqueous HCl, saturated aqueous NaHCO₃, brine, dried (MgSO₄) and concentrated under reduced pressure. The residue and Pd/C (31.7 mg, 10 wt %, 0.030 mmol) in MeOH (3 mL) was sparged with hydrogen for 10 min and then stirred at RT overnight under a balloon atmosphere of H₂. The reaction mixture was filtered through Celite and washed with methanol. The filtrate was evaporated to dryness and the residue purified by chromatography (ISCO, 4 g silica, 0-100% EtOAc/Hex) to give the title compound as a clear, colorless oil (54.6 mg, 63% over 2 steps). LCMS m/z=311 [M+Na]⁺.

### Intermediate A85. tert-butyl 2-(3-amino-5-fluorophenyl) piperidine-1-carboxylate

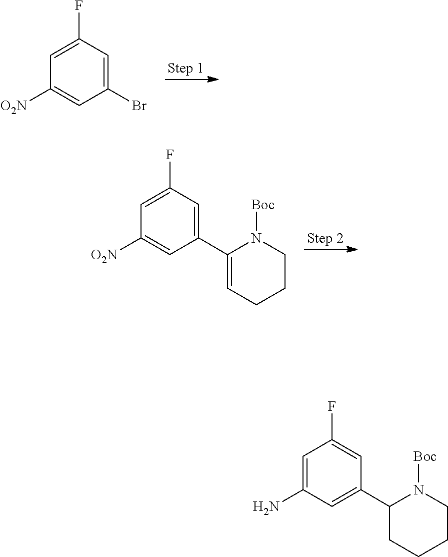 [SMILES: O=[N+]([O-])c1cc(F)cc(Br)c1 ; CC(C)(C)OC(=O)N1CCCC=C1c1cc(F)cc([N+](=O)[O-])c1 ; CC(C)(C)OC(=O)N1CCCCC1c1cc(N)cc(F)c1]

### Step 1. Synthesis of tert-butyl 6-(3-fluoro-5-nitrophenyl)-3,4-dihydropyridine-1 (2H)-carboxylate

A mixture of 1-bromo-3-fluoro-5-nitrobenzene (219 mg, 1.00 mmol), tert-butyl 6-(4,4,5,5-tetramethyl-1,3,2-dioxaborolan-2-yl)-3,4-dihydropyridine-1 (2H)-carboxylate (413 mg, 1.30 mmol), tBuXPhos (33.1 mg, 0.075 mmol), PdCl₂ (PPh₃)₂ (35.6 mg, 0.050 mmol) in dioxane (4 mL) and saturated aqueous sodium carbonate (1 mL) was sparged with N₂ for 10 min and heated to 80° C. overnight. The reaction was filtered through Celite, washed with EtOAc and concentrated under reduced pressure. The residue was purified by chromatography (ISCO 12 g silica, 0-80% EtOAc/Hex) to afford the title compound as a clear yellow oil (83.1 mg, 26%). LCMS m/z=345 [M+Na]⁺.

### Step 2. Synthesis of tert-butyl 2-(3-amino-5-fluorophenyl) piperidine-1-carboxylate

A mixture of tert-butyl 6-(3-nitrophenyl)-3,4-dihydropyridine-1 (2H)-carboxylate (Step 1, 78.4 mg, 0.24 mmol) and Pd/C (27.5 mg, 10 wt %, 0.024 mmol) in MeOH (5 mL) was charged with H₂ with 4 fill/vacuum cycles. The flask was pressurized to 40 psi H₂, sealed and heated to 60° C. overnight. The reaction was filtered through Celite, washed with MeOH and the filtrate was concentrated to provide the title compound as a yellow oil (66.5 mg, 93%). The material was used in the next step without purification. LCMS m/z=317 [M+Na]⁺.

### Intermediate A85. tert-butyl(S)-2-(3-amino-2-fluorophenyl) pyrrolidine-1-carboxylate or tert-butyl(R)-2-(3-amino-2-fluorophenyl) pyrrolidine-1-carboxylate

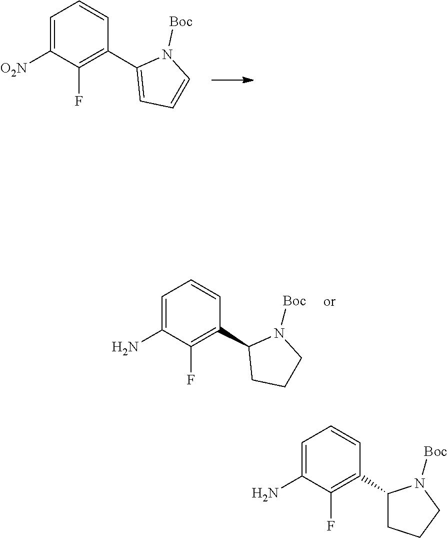 [SMILES: CC(C)(C)OC(=O)n1cccc1-c1cccc([N+](=O)[O-])c1F ; CC(C)(C)OC(=O)N1CCC[C@H]1c1cccc(N)c1F ; CC(C)(C)OC(=O)N1CCC[C@@H]1c1cccc(N)c1F]

Pd/C (200 mg, 10% purity) was added to tert-butyl 2-(2-fluoro-3-nitrophenyl)-1H-pyrrole-1-carboxylate (Intermediate A76 and A77, Step 1, 200 mg, 0.653 mmol) in MeOH (2 mL) and the mixture stirred at 60° C. for 20 h under H₂ (50 psi). The reaction mixture was filtered and the filtrate evaporated to dryness and the residue purified by prep-TLC (50% EtOAc/PE/) and by SFC (DAICEL CHIRALCEL OJ, 250×30 mm, 10 μm); 25% IPA (0.1% NH₄OH) in CO₂) to afford:

Peak 1, Intermediate A85. tert-butyl(S)-2-(3-amino-2-fluorophenyl) pyrrolidine-1-carboxylate or tert-butyl(R)-2-(3-amino-2-fluorophenyl) pyrrolidine-1-carboxylate (white solid, 20 mg, 11%). LCMS m/z=225 [M−56+1]⁺.

### Intermediate B1. methyl 4-(bis(4-methoxybenzyl)amino)-6-oxo-1,6-dihydropyrimidine-5-carboxylate

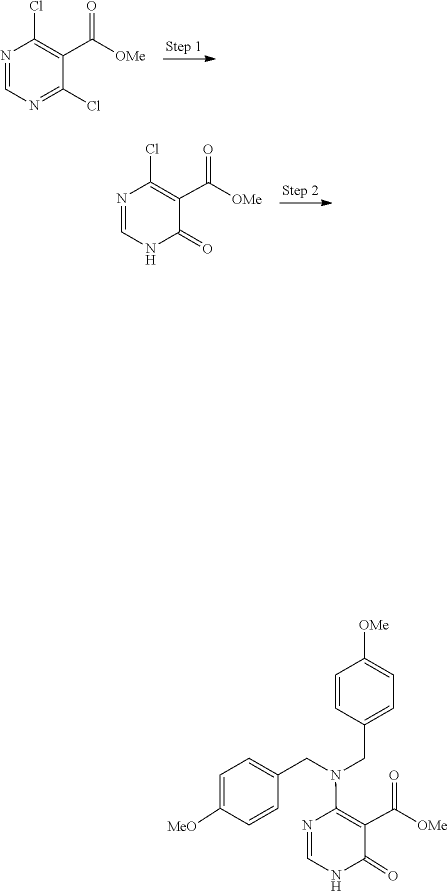 [SMILES: COC(=O)c1c(Cl)ncnc1Cl ; COC(=O)c1c(Cl)nc[nH]c1=O ; COC(=O)c1c(N(Cc2ccc(OC)cc2)Cc2ccc(OC)cc2)nc[nH]c1=O]

### Step 1. Synthesis of methyl 4-chloro-6-oxo-1,6-dihydropyrimidine-5-carboxylate

A mixture of methyl 4,6-dichloropyrimidine-5-carboxylate (670 g, 3.10 mol, 95.7% purity) and AcOH (2.51 kg, 41.8 mol) was stirred at 120° C. for 24 h under N₂. The reaction mixture was concentrated under reduced pressure and the residue triturated with EtOAc (500 mL) at 25° C. for 16 h. The solids were collected by filtration to afford the title compound as a brown solid (1.50 kg, crude) which was used without further purification. ¹H NMR (400 MHz, DMSO-d₆) δ: 8.31 (s, 1H), 3.81 (s, 3H).

### Step 2. Synthesis of methyl 4-(bis(4-methoxybenzyl)amino)-6-oxo-1,6-dihydropyrimidine-5-carboxylate

A 5 L three necked flash was charged with DMSO (2.50 L). To this was added sequentially at 20-25° C., methyl 4-chloro-6-oxo-1,6-dihydropyrimidine-5-carboxylate (300 g, 1.22 mol, 76.5% purity), bis(4-methoxybenzyl)amine (313 g, 1.22 mol), DIPEA (315 g, 2.43 mol) and the resulting mixture stirred at 75-80° C. for 5 h. The reaction mixture was cooled to 20-25° C., poured into water (8 L) and extracted with DCM (2×5 L). The combined organics were washed with brine (3×1.5 L) and separate, dried (Na₂SO₄) and concentrated under reduced pressure. The residue was triturated with MTBE (1 L) at 15-20° C. for 1 h and the solids collected by filtration and the filter cake dried under vacuum to afford the title compound as a yellow solid (421 g, 80.4%). LCMS m/z=410 [M+H]⁺.

### Intermediate B2. Synthesis of methyl 4-(bis(2,4-dimethoxybenzyl)amino)-6-oxo-1,6-dihydropyrimidine-5-carboxylate

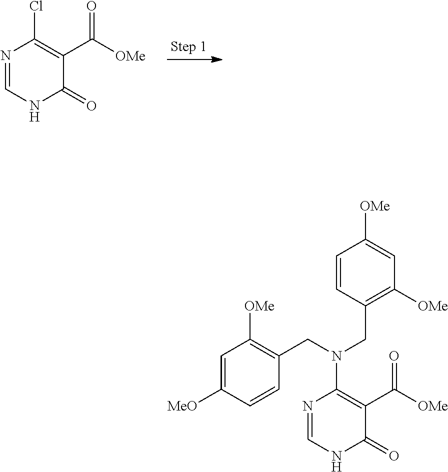 [SMILES: COC(=O)c1c(Cl)nc[nH]c1=O ; COC(=O)c1c(N(Cc2ccc(OC)cc2OC)Cc2ccc(OC)cc2OC)nc[nH]c1=O]

To a solution of methyl 4-chloro-6-oxo-1,6-dihydropyrimidine-5-carboxylate (Intermediate B1, Step 1, 1 g, 5.30 mmol) and bis(2,4-dimethoxybenzyl)amine (3.37 g, 10.6 mmol) in DMSO (8 mL) was added DIPEA (2.74 g, 21.2 mmol) and the mixture stirred at 100° C. for 12 h. Water (10 mL) was added and the mixture extracted with EtOAc (3×10 mL). The combined organics were dried (Na₂SO₄) and concentrated under reduced pressure and the residue was purified by column chromatography (SiO₂, 0-100% EtOAc/PE) to give the title compound as a yellow oil (2.4 g, 96%). LCMS m/z=470 [M+H]⁺.

### Intermediate B3. Methyl 4-amino-1-((S)-2-chloro-6-methylphenyl)-6-oxo-1,6-dihydropyrimidine-5-carboxylate

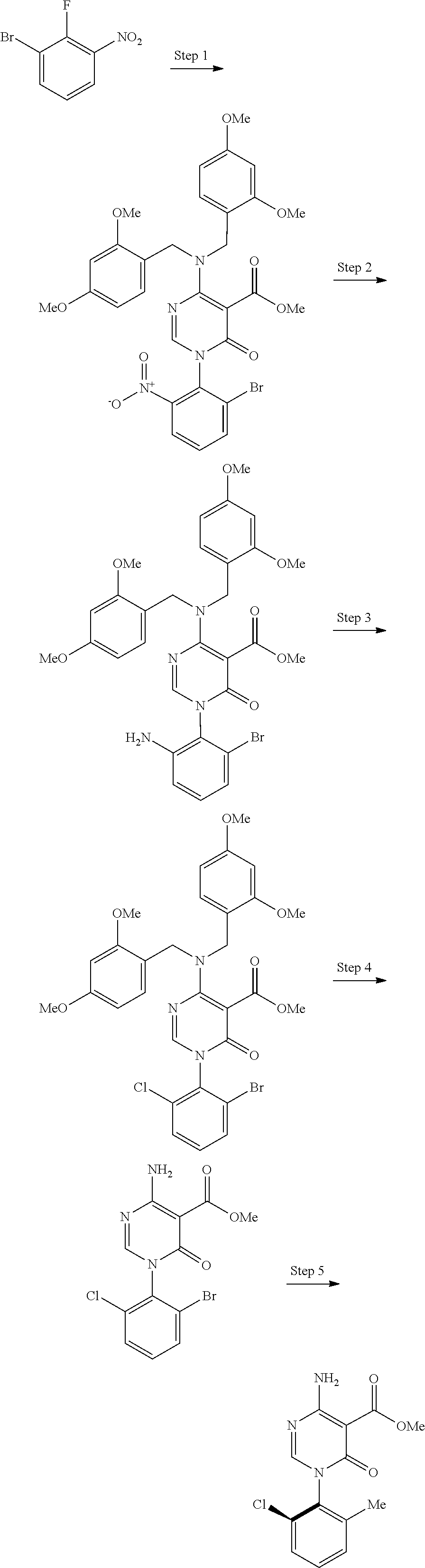

### Step 1. Synthesis of methyl 4-(bis(2,4-dimethoxybenzyl)amino)-1-(2-bromo-6-nitrophenyl)-6-oxo-1,6-dihydropyrimidine-5-carboxylate

K₂CO₃ (6.75 g, 48.9 mmol) was added to a mixture of 1-bromo-2-fluoro-3-nitro-benzene (6.45 g, 29.3 mmol) and methyl 4-(bis(2,4-dimethoxybenzyl)amino)-6-oxo-1,6-dihydropyrimidine-5-carboxylate (Intermediate B2, 11.47 g, 24.4 mmol) in DMF (80 mL) and the mixture stirred at 80° C. for 1 h. The solids were removed by filtration and H₂O (100 mL) added to the filtrate. The resulting solid was collected by filtration to afford the title compound as a yellow solid (14.5 g, 89%). LCMS m/z=669 [M+H]⁺.

### Step 2. Synthesis of methyl 1-(2-amino-6-bromophenyl)-4-(bis(2,4-dimethoxybenzyl)amino)-6-oxo-1,6-dihydropyrimidine-5-carboxylate

Fe (12.1 g, 216 mmol) and NH₄Cl (11.6 g, 216 mmol) were added to a mixture of methyl 4-(bis(2,4-dimethoxybenzyl)amino)-1-(2-bromo-6-nitrophenyl)-6-oxo-1,6-dihydropyrimidine-5-carboxylate (14.5 g, 21.7 mmol) in THF (100 mL), H₂O (20 mL) and EtOH (30 mL) and the mixture stirred at 80° C. for 1 h. The solids were removed by filtration and H₂O (100 mL) added to the filtrate. The resulting solid was collected to afford the title compound as a yellow solid (13 g, 94%). LCMS m/z=607 [M+H]⁺.

### Step 3. Synthesis of methyl 4-(bis(2,4-dimethoxybenzyl)amino)-1-(2-bromo-6-chlorophenyl)-6-oxo-1,6-dihydropyrimidine-5-carboxylate

A mixture of methyl 1-(2-amino-6-bromophenyl)-4-(bis(2,4-dimethoxybenzyl)amino)-6-oxo-1,6-dihydropyrimidine-5-carboxylate (5 g, 7.82 mmol, 1 eq), CuCl (1.55 g, 15.6 mmol), CuCl₂ (2.10 g, 15.6 mmol) in CH₃CN (40 mL) was added isopentyl nitrite (2.75 g, 23.46 mmol, 3.16 mL) in CH₃CN (2 ml) and then the mixture stirred at 20° C. for 1 h under N₂. The reaction mixture was evaporated under a flow of N₂. The residue was diluted with H₂O (50 mL) and extracted with EtOAc (4×50 mL). The combined organics were dried (Na₂SO₄) and evaporated to dryness in vacuo and the residue purified by column chromatography (SiO₂, 0-100% EtOAc/PE) to the title compound as a yellow oil (13 g, crude). LCMS m/z=660 [M+H].

### Step 4. Synthesis of methyl 4-amino-1-(2-bromo-6-chlorophenyl)-6-oxo-1,6-dihydropyrimidine-5-carboxylate

A mixture of methyl 4-(bis(2,4-dimethoxybenzyl)amino)-1-(2-bromo-6-chlorophenyl)-6-oxo-1,6-dihydropyrimidine-5-carboxylate (11.5 g, 17.5 mmol) in TFA (5 mL) and DCM (10 mL) was stirred at 20° C. for 1 h. The reaction mixture was evaporated to dryness under a stream of N₂. The residue was diluted with H₂O (50 mL) and saturated aq. Na₂CO₃ added until pH=8 and the mixture was extracted with EtOAc (4×50 mL). The combined organics were dried (Na₂SO₄) and evaporated to dryness in vacuo. The residue was purified by column chromatography (SiO₂, 0-100% EtOAc/PE) to give the title compound as a yellow solid (3.8 g, 61%). LCMS m/z=360 [M+H]⁺.

### Step 5. Synthesis of methyl 4-amino-1-(2-chloro-6-methylphenyl)-6-oxo-1,6-dihydropyrimidine-5-carboxylate

A mixture of methyl 4-amino-1-(2-bromo-6-chloro-phenyl)-6-oxo-pyrimidine-5-carboxylate (2.02 g, 5.63 mmol), 2,4,6-trimethyl-1,3,5,2,4,6-trioxatriborinane (2.12 g, 8.45 mmol, 2.36 mL, 50% purity), Pd(dppf)Cl₂ (412.2 mg, 0.563 mmol) and K₂CO₃ (1.56 g, 11.3 mmol) in dioxane (20 mL) was degassed with N₂ (3×) and stirred at 100° C. for 1.5 h under N₂. The reaction mixture was filtered and concentrated under reduced pressure and the residue purified by column chromatography (SiO₂, 0-66% EtOAc/PE) to give (rac)-methyl 4-amino-1-(2-chloro-6-methylphenyl)-6-oxo-1,6-dihydropyrimidine-5-carboxylate. The racemate was separated by chiral SFC (Diacel Chiralpak IG, 250×30 mm, 10 μm; 35% (0.1% NH₄OH/IPA) in CO₂) to afford:

Peak 1, Intermediate B3: methyl 4-amino-1-((S)-2-chloro-6-methylphenyl)-6-oxo-1,6-dihydropyrimidine-5-carboxylate (yellow solid, 400 mg, 17.3%). LCMS m/z=294 [M+H]⁺.

### Intermediate B4. Methyl 4-amino-1-((S)-2-chloro-4-methoxy-6-methylphenyl)-6-oxo-1,6-dihydropyrimidine-5-carboxylate

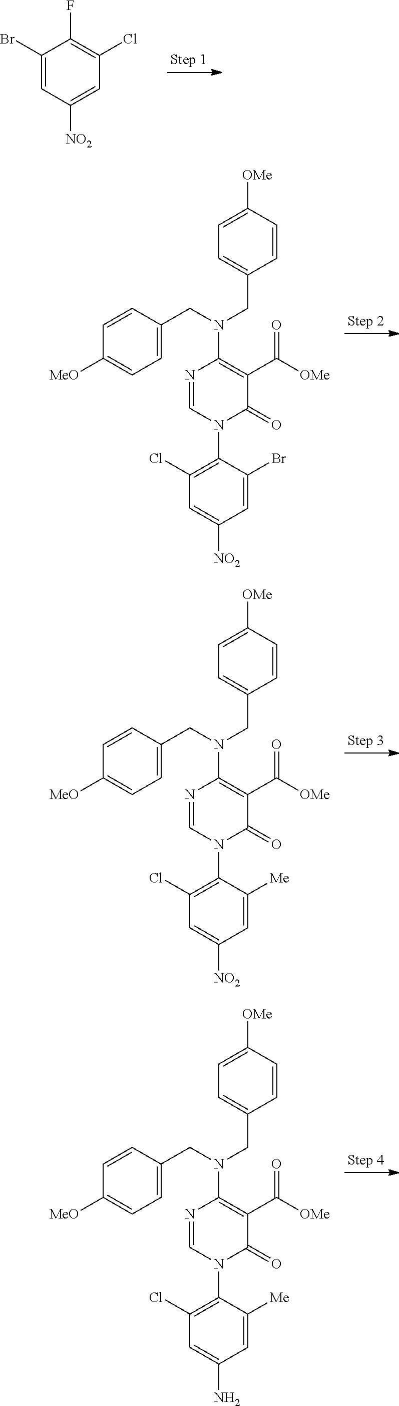

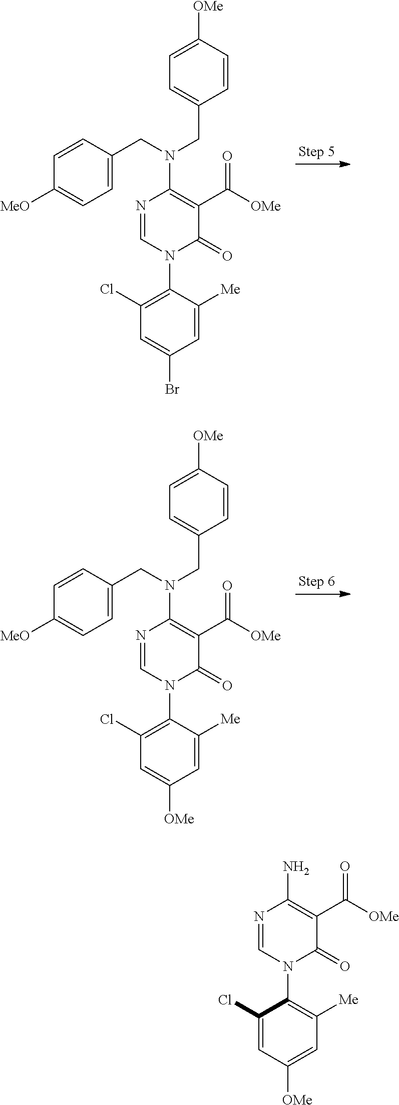

### Step 1. Synthesis of methyl 4-(bis(4-methoxybenzyl)amino)-1-(2-bromo-6-chloro-4-nitrophenyl)-6-oxo-1,6-dihydropyrimidine-5-carboxylate

DIPEA (10.1 g, 78.2 mmol) was added to a solution of methyl 4-(bis(4-methoxybenzyl)amino)-6-oxo-1,6-dihydropyrimidine-5-carboxylate (Intermediate B1, 6.4 g, 15.63 mmol) and 1-bromo-3-chloro-2-fluoro-5-nitro-benzene (3.98 g, 15.63 mmol) in DMSO (50 mL) and the mixture stirred at 100° C. for 8 h. The reaction mixture was treated with H₂O (30 mL), filtered and the filter cake dried under reduced pressure to give the title compound as a brown solid (10.2 g, crude). LCMS m/z=645 [M+H]⁺.

### Step 2. Synthesis of methyl 4-(bis(4-methoxybenzyl)amino)-1-(2-chloro-6-methyl-4-nitrophenyl)-6-oxo-1,6-dihydropyrimidine-5-carboxylate

To a solution of 2,4,6-trimethyl-1,3,5,2,4,6-trioxatriborinane (4.06 g, 16.15 mmol, 4.52 mL, 50% purity), methyl 4-(bis(4-methoxybenzyl)amino)-1-(2-bromo-6-chloro-4-nitrophenyl)-6-oxo-1,6-dihydropyrimidine-5-carboxylate (8 g, 12.42 mmol) in dioxane (100 mL) was added Pd(dppf)Cl₂ (455 mg, 0.621 mmol) and K₂CO₃ (3.43 g, 24.9 mmol) and the mixture stirred at 100° C. for 2 h under N₂. The reaction mixture was filtered and the filtrate was concentrated under reduced pressure and the residue purified by MPLC (SiO₂, 0-50% THF/PE) to afford the title compound as a brown solid (8.26 g, crude). LCMS m/z=579 [M+H]⁺.

### Step 3. Synthesis of methyl 1-(4-amino-2-chloro-6-methylphenyl)-4-(bis(4-methoxybenzyl)amino)-6-oxo-1,6-dihydropyrimidine-5-carboxylate

A mixture of methyl 4-(bis(4-methoxybenzyl)amino)-1-(2-chloro-6-methyl-4-nitrophenyl)-6-oxo-1,6-dihydropyrimidine-5-carboxylate (8.2 g, 14.2 mmol), NH₄Cl (7.58 g, 142 mmol), Fe (7.91 g, 142 mmol) in THF (40 mL), EtOH (40 mL) and H₂O (10 mL) was stirred at 80° C. for 2 h. The reaction mixture was filtered and the filtrate was concentrated under reduced pressure to give a residue which was diluted with H₂O (80 mL) and extracted with EtOAc (3×60 mL). The combined organics were dried (Na₂SO₄) and evaporated to dryness under reduced pressure to give the title compound as a brown solid (7.05 g, 90%). LCMS m/z=549 [M+H]⁺.

### Step 4. Synthesis of methyl 4-(bis(4-methoxybenzyl)amino)-1-(4-bromo-2-chloro-6-methylphenyl)-6-oxo-1,6-dihydropyrimidine-5-carboxylate

To a solution of methyl 1-(4-amino-2-chloro-6-methylphenyl)-4-(bis(4-methoxybenzyl)amino)-6-oxo-1,6-dihydropyrimidine-5-carboxylate (7 g, 12.8 mmol) in MeCN (150 mL) was added TBAB (24.66 g, 76.50 mmol), CuBr (2.19 g, 15.3 mmol), ((1S,4R)-7,7-dimethyl-2-oxobicyclo[2.2.1]heptan-1-yl) methanesulfonic acid (8.89 g, 38.3 mmol) and NaNO₂ (2.64 g, 38.3 mmol) and the mixture stirred at 25° C. for 2 h. The reaction mixture was concentrated under reduced pressure and the residue diluted with H₂O (100 mL) and extracted with EtOAc (3×60 mL). The combine extracts were dried (Na₂SO₄) and concentrated under reduced pressure. The residue was purified by MPLC (SiO₂, 0-50% EtOAc/PE) to give the title compound as a yellow solid (5 g, 64%). LCMS m/z=614 [M+H]⁺.

### Step 5. Synthesis of methyl 4-(bis(4-methoxybenzyl)amino)-1-(2-chloro-4-methoxy-6-methylphenyl)-6-oxo-1,6-dihydropyrimidine-5-carboxylate

To a solution of methyl 4-(bis(4-methoxybenzyl)amino)-1-(4-bromo-2-chloro-6-methylphenyl)-6-oxo-1,6-dihydropyrimidine-5-carboxylate (2 g, 3.26 mmol) in MeOH (6 mL) and toluene (6 mL) was added Pd₂ (dba)₃ (149.4 mg, 0.163 mmol), t-Bu Xphos (138.6 mg, 0.326 mmol) and K₃PO₄ (1.04 g, 4.89 mmol) and the mixture stirred at 80° C. for 1 h under N₂. The reaction mixture was concentrated under reduced pressure to give a residue which was purified by MPLC (SiO₂, 0-50% EtOAc/PE) to give the title compound as a brown solid (870 mg, 1.54 mmol, 47%). LCMS m/z=564 [M+H]⁺.

### Step 6. Synthesis of (S)-methyl 4-amino-1-(2-chloro-4-methoxy-6-methylphenyl)-6-oxo-1,6-dihydropyrimidine-5-carboxylate

A mixture methyl 4-(bis(4-methoxybenzyl)amino)-1-(2-chloro-4-methoxy-6-methylphenyl)-6-oxo-1,6-dihydropyrimidine-5-carboxylate (870 mg, 1.54 mmol) in TFA (8 mL) was stirred at 80° C. for 0.5 h. The reaction mixture was concentrated under reduced pressure and the residue adjusted with NaHCO₃ to pH 7-8. The mixture was diluted with H₂O (20 mL) and extracted with EtOAc (3×10 mL), dried (Na₂SO₄) and concentrated under reduced pressure. The residue was purified by MPLC (SiO₂, 0-50% EtOAc/PE) followed by chiral SFC (DAICEL CHIRALPAK AD, 250×30 mm, 10 μm); 27% (0.1% NH₄OH/IPA) in CO₂) to afford:

Peak 1, Intermediate B4. (S)-methyl 4-amino-1-(2-chloro-4-methoxy-6-methylphenyl)-6-oxo-1,6-dihydropyrimidine-5-carboxylate (white solid, 187 mg, 37%). LCMS m/z=324 [M+H]⁺.

### Intermediate B5. (S)-methyl 4-amino-1-(2-chloro-4-ethoxy-6-methylphenyl)-6-oxo-1,6-dihydropyrimidine-5-carboxylate

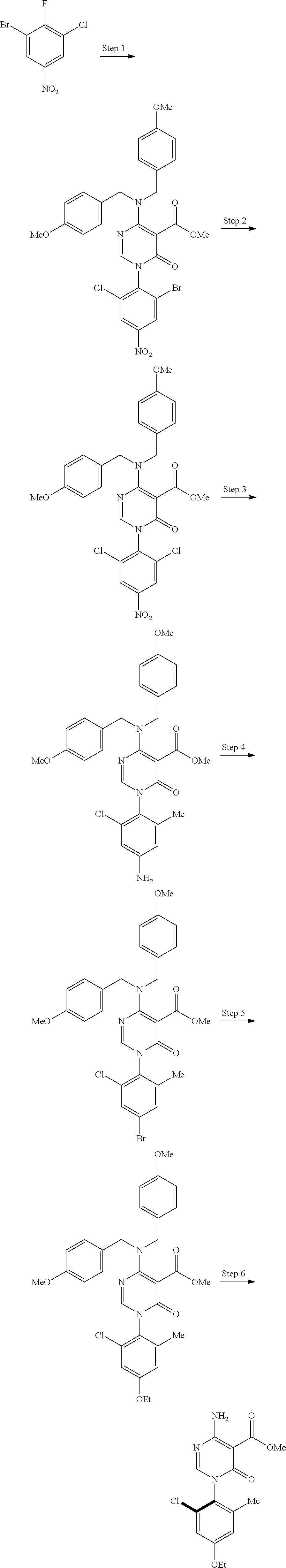

The title compounds were prepared using an analogous method to that described for Intermediate B4. chiral SFC (DAICEL CHIRALPAK IC, 250×30 mm, 10 μm); 45% (0.1% NH₄OH/IPA) in CO₂) to afford:

Peak 1, Intermediate B5. (S)-methyl 4-amino-1-(2-chloro-4-ethoxy-6-methylphenyl)-6-oxo-1,6-dihydropyrimidine-5-carboxylate (white oil, 730 mg, 46%). LCMS m/z=338 [M+H]⁺.

### Intermediate B6. Methyl 4-amino-1-(4-bromo-2,6-dichlorophenyl)-6-oxo-1,6-dihydropyrimidine-5-carboxylate

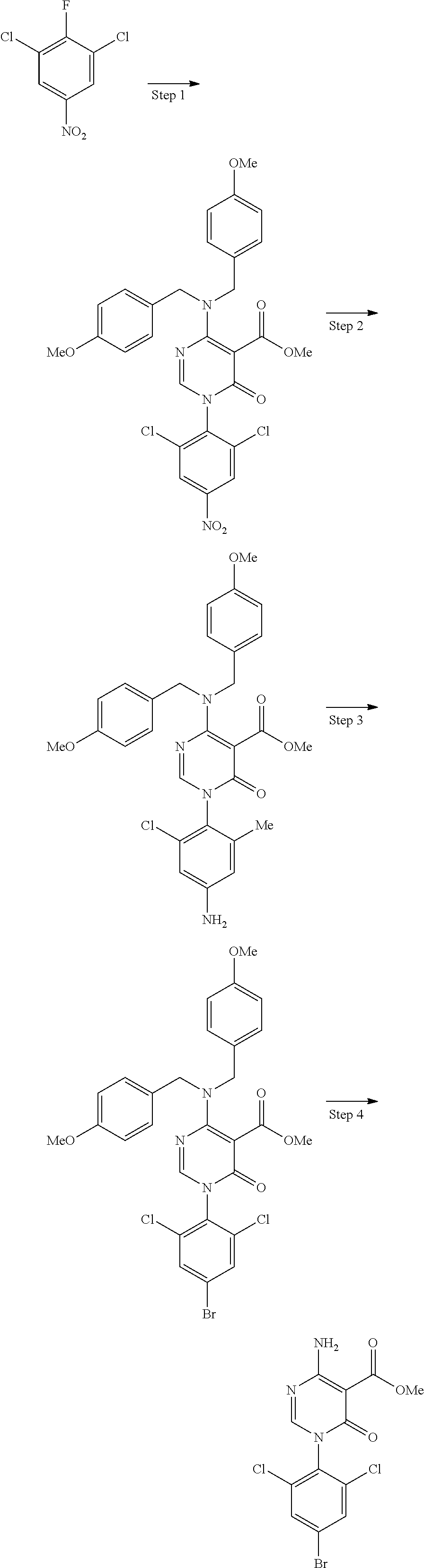

### Step 1. Synthesis of methyl 4-(bis(4-methoxybenzyl)amino)-1-(2,6-dichloro-4-nitrophenyl)-6-oxo-1,6-dihydropyrimidine-5-carboxylate

DIPEA (1.58 g, 12.21 mmol) was added to a mixture of methyl 4-(bis(4-methoxybenzyl)amino)-6-oxo-1,6-dihydropyrimidine-5-carboxylate (Intermediate B1, 2.5 g, 6.11 mmol) and 1,3-dichloro-2-fluoro-5-nitro-benzene (5.13 g, 24.42 mmol) in DMSO (15 mL) and the mixture stirred at 100° C. for 5 h. The reaction mixture was partitioned between EtOAc (30 mL) and H₂O (20 mL) and the aqueous phase extracted with EtOAc (3×30 mL). The combined organics were dried (Na₂SO₄) and evaporated to dryness in vacuo. The residue was purified by MPLC (SiO₂, 0-50% EtOAc/PE) to give the title compound as a yellow solid (4 g, crude). LCMS m/z=599 [M+H]⁺.

### Step 2. Synthesis of methyl 1-(4-amino-2,6-dichlorophenyl)-4-(bis(4-methoxybenzyl)amino)-6-oxo-1,6-dihydropyrimidine-5-carboxylate

Pd/C (0.2 g, 10% purity) was added to a solution of methyl 4-(bis(4-methoxybenzyl)amino)-1-(2,6-dichloro-4-nitrophenyl)-6-oxo-1,6-dihydropyrimidine-5-carboxylate (Step 1, 4 g, 6.67 mmol) in MeOH (5 mL) and EtOAc (5 mL) and the mixture was stirred at 15° C. for 1 hr under H₂ (15 psi). The reaction mixture was filtered and the filtrated evaporated to dryness in vacuo to afford the title compound as a yellow solid (2.2 g) which was used without further purification.

### Step 3. Synthesis of methyl 4-(bis(4-methoxybenzyl)amino)-1-(4-bromo-2,6-dichlorophenyl)-6-oxo-1,6-dihydropyrimidine-5-carboxylate

A mixture of methyl 1-(4-amino-2,6-dichlorophenyl)-4-(bis(4-methoxybenzyl)amino)-6-oxo-1,6-dihydropyrimidine-5-carboxylate (Intermediate B6 Step 2, 3.5 g, 6.15 mmol), CuBr (1.06 g, 7.38 mmol), TBAB (11.89 g, 36.9 mmol), (1R,3S)-1,2,2-trimethylcyclopentane-1,3-dicarboxylic acid (3.69 g, 18.44 mmol) and NaNO₂ (1.27 g, 18.44 mmol) in MeCN (40 mL) was stirred at 15° C. for 12 h. The mixture was concentrated under reduced pressure and the residue partitioned between EtOAc (30 mL) and H₂O (20 mL). The aqueous phase was further extracted with EtOAc (3×30 mL). The combined organics were dried (Na₂SO₄) and evaporated to dryness in vacuo. The residue was purified by MPLC (SiO₂, 0-50% EtoAc/PE) to give the title compound as a yellow solid (1.5 g, 38.5%). LCMS m/z=634 [M+H]⁺.

### Step 4. Synthesis of methyl 4-amino-1-(4-bromo-2,6-dichlorophenyl)-6-oxo-1,6-dihydropyrimidine-5-carboxylate

Methyl 4-(bis(4-methoxybenzyl)amino)-1-(4-bromo-2,6-dichlorophenyl)-6-oxo-1,6-dihydropyrimidine-5-carboxylate (Step 3, 1.5 g, 2.37 mmol) in TFA (2 mL) was stirred at 80° C. for 1 h. The reaction mixture was concentrated under reduced pressure and the residue partitioned between EtOAc (20 mL) and sat. aq. Na₂CO₃ (15 mL). The aqueous phase was extracted with EtOAc (3×20 mL) and the combined organics were dried (Na₂SO₄) and evaporated to dryness in vacuo. The residue was purified by MPLC (SiO₂, 0-50% EtOAc/PE) to give the title compound as a yellow solid (800 mg, crude). LCMS m/z=394 [M+H]⁺.

### Intermediate B7. Methyl 4-amino-1-(2,6-dichloro-4-propoxyphenyl)-6-oxo-1,6-dihydropyrimidine-5-carboxylate

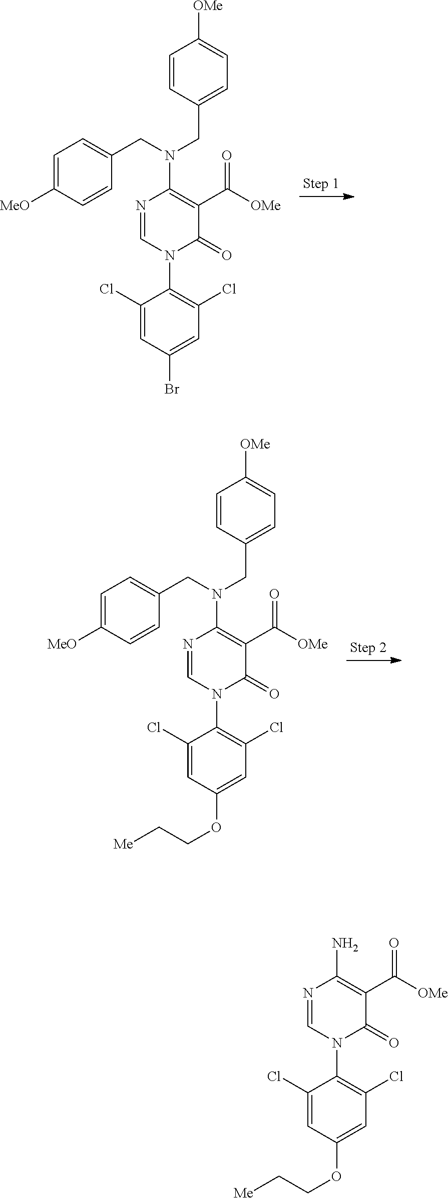 [SMILES: COC(=O)c1c(N(Cc2ccc(OC)cc2)Cc2ccc(OC)cc2)ncn(-c2c(Cl)cc(Br)cc2Cl)c1=O ; CCCOc1cc(Cl)c(-n2cnc(N(Cc3ccc(OC)cc3)Cc3ccc(OC)cc3)c(C(=O)OC)c2=O)c(Cl)c1 ; CCCOc1cc(Cl)c(-n2cnc(N)c(C(=O)OC)c2=O)c(Cl)c1]

### Step 1. Synthesis of methyl 4-(bis(4-methoxybenzyl)amino)-1-(2,6-dichloro-4-propoxyphenyl)-6-oxo-1,6-dihydropyrimidine-5-carboxylate

To a solution of methyl 4-(bis(4-methoxybenzyl)amino)-1-(4-bromo-2,6-dichlorophenyl)-6-oxo-1,6-dihydropyrimidine-5-carboxylate (Intermediate B6 Step 3, 500 mg, 0.789 mmol) in toluene (1 mL) and 1-propanol (1 mL) was added t-Bu Xphos (33.5 mg, 0,079 mmol) and Pd₂ (dba)₃ (72.3 mg, 0.079 mmol), K₃PO₄ (251 mg, 1.18 mmol) and the mixture stirred at 80° C. for 1 h. The reaction mixture was filtered and the filtrate was concentrated under reduced pressure and the residue was purified by column chromatography (SiO₂, 0-25% EtOAc/PE) to give the title compound as a brown oil (300 mg, 62%). LCMS m/z=612 [M+H]⁺.

### Step 2. Synthesis of methyl 4-amino-1-(2,6-dichloro-4-propoxyphenyl)-6-oxo-1,6-dihydropyrimidine-5-carboxylate

A solution of methyl 4-(bis(4-methoxybenzyl)amino)-1-(2,6-dichloro-4-propoxyphenyl)-6-oxo-1,6-dihydropyrimidine-5-carboxylate (Step 1, 270 mg, 0.441 mmol) in TFA (4 mL) was stirred at 80° C. for 1 h. The reaction mixture was concentrated under N₂ and the residue diluted with H₂O (20 mL) and sat. aq. Na₂CO₃ was added until pH-8 and the mixture was extracted with EtOAc (3×20 mL). The combined organics were dried (Na₂SO₄) and concentrated under reduced pressure and the residue purified by column chromatography (SiO₂, 0-33% EtOAc/PE) to give the title compound as a white solid (110 mg, 67%). LCMS m/z=372 [M+H]⁺.

### Intermediate B8. Methyl 4-amino-1-(2,6-dichloro-4-(dimethylamino)phenyl)-6-oxo-1,6-dihydropyrimidine-5-carboxylate

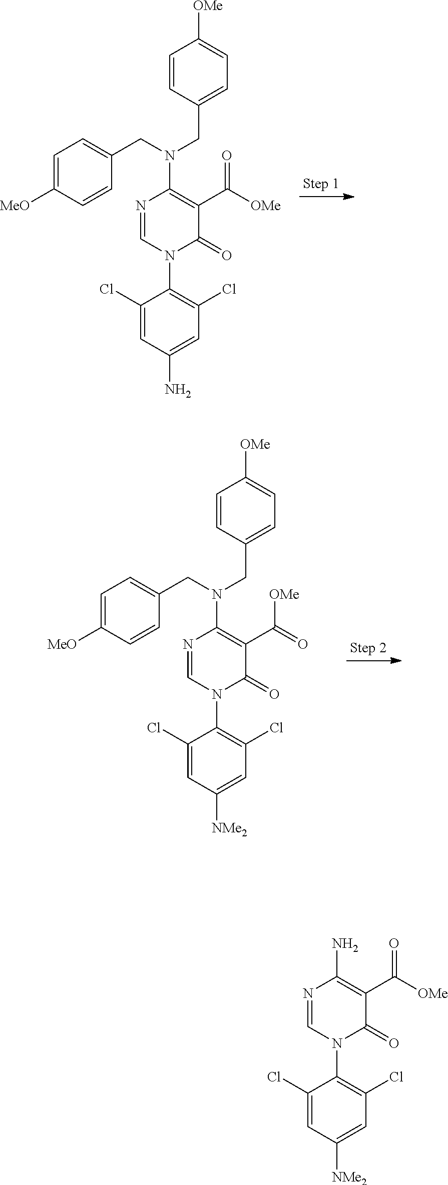 [SMILES: COC(=O)c1c(N(Cc2ccc(OC)cc2)Cc2ccc(OC)cc2)ncn(-c2c(Cl)cc(N)cc2Cl)c1=O ; COC(=O)c1c(N(Cc2ccc(OC)cc2)Cc2ccc(OC)cc2)ncn(-c2c(Cl)cc(N(C)C)cc2Cl)c1=O ; COC(=O)c1c(N)ncn(-c2c(Cl)cc(N(C)C)cc2Cl)c1=O]

### Step 1. Synthesis of methyl 4-(bis(4-methoxybenzyl)amino)-1-(2,6-dichloro-4-(dimethylamino)phenyl)-6-oxo-1,6-dihydropyrimidine-5-carboxylate

To a solution of methyl 1-(4-amino-2,6-dichlorophenyl)-4-(bis(4-methoxybenzyl)amino)-6-oxo-1,6-dihydropyrimidine-5-carboxylate (Intermediate B6, Step 2; 500 mg, 0.88 mmol) in MeOH (5 mL) was added AcOH (52.73 mg, 0.88 mmol) to pH=5, and then HCHO (356 mg, 4.39 mmol) was added and the mixture stirred at 25° C. for 1 h. To this was added NaBH₃CN (82.8 mg, 1.32 mmol) and the mixture stirred at 25° C. for 1 h. The reaction mixture was diluted with H₂O (5 mL) and extracted with EtOAc (3×4 mL). The combined organics were washed with brine (2×3 mL), dried (Na₂SO₄) and concentrated under reduced pressure to give the title compound as a white solid (500 mg). LCMS m/z=597 [M+H]⁺.

### Step 2. Synthesis of methyl 4-amino-1-(2,6-dichloro-4-(dimethylamino)phenyl)-6-oxo-1,6-dihydropyrimidine-5-carboxylate

A solution of methyl 4-(bis(4-methoxybenzyl)amino)-1-(2,6-dichloro-4-(dimethylamino)phenyl)-6-oxo-1,6-dihydropyrimidine-5-carboxylate (Step 1, 450 mg, 0.75 mmol) in TFA (5 mL) was stirred at 80° C. for 1 h. The reaction mixture was quenched by addition saturated aqueous Na₂CO₃ solution (3 mL) at 0° C. and extracted with EtOAc (3×4 mL). The combined organics were dried (Na₂SO₄) and concentrated under reduced pressure. The residue was purified by prep-TLC to give the title compound as a white solid (50 mg, 18%). LCMS m/z=357 [M+H]⁺.

### Intermediate B9. Synthesis of methyl 4-amino-1-(2,6-dichlorophenyl)-6-oxo-1,6-dihydropyrimidine-5-carboxylate

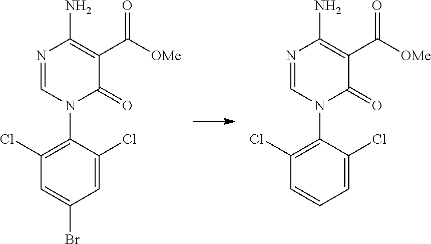 [SMILES: COC(=O)c1c(N)ncn(-c2c(Cl)cc(Br)cc2Cl)c1=O ; COC(=O)c1c(N)ncn(-c2c(Cl)cccc2Cl)c1=O]

To a methyl 4-amino-1-(4-bromo-2,6-dichlorophenyl)-6-oxo-1,6-dihydropyrimidine-5-carboxylate (Intermediate B6, 150 mg, 0.382 mmol) in MeOH (10 mL) and THF (1 mL) was added Pd/C (0.2 g, 10% purity) and the mixture stirred at 25° C. for 1 h under H₂ (15 psi). The reaction mixture was filtered and the filtrate concentrated under reduced pressure. The residue was purified by prep-TLC (50% EtOAc/PE) to give the title compound as a yellow solid (70 mg, 58.4%). LCMS m/z=314 [M+H]⁺.

### Intermediate B10. Synthesis of methyl 4-amino-1-(2,6-dichloro-4-methoxyphenyl)-6-oxo-1,6-dihydropyrimidine-5-carboxylate

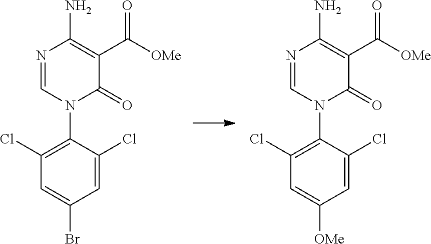 [SMILES: COC(=O)c1c(N)ncn(-c2c(Cl)cc(Br)cc2Cl)c1=O ; COC(=O)c1c(N)ncn(-c2c(Cl)cc(OC)cc2Cl)c1=O]

A mixture of methyl 4-amino-1-(4-bromo-2,6-dichlorophenyl)-6-oxo-1,6-dihydropyrimidine-5-carboxylate (Intermediate B6, 500 mg, 1.27 mmol), Cs₂CO₃ (829 mg, 2.54 mmol) and RockPhos Pd G3 (107 mg, 0.127 mmol) in dioxane (5 mL) and MeOH (0.20 mL) was stirred at 80° C. for 2 h under N₂. The reaction mixture was filtered and the filtrate evaporated to dryness under reduced pressure and the residue purified by prep-HPLC (Xbridge Prep OBD C18, 150×40 mm, 10 μm; 20-50% MeCN/H₂O (10 mM NH₄HCO₃)) to give the title compound as a white solid (150 mg, 34%). LCMS m/z=344 [M+H]⁺.

### Intermediate B11. Synthesis of methyl 4-amino-1-(2,6-dichloro-4-cyclopropoxyphenyl)-6-oxo-1,6-dihydropyrimidine-5-carboxylate

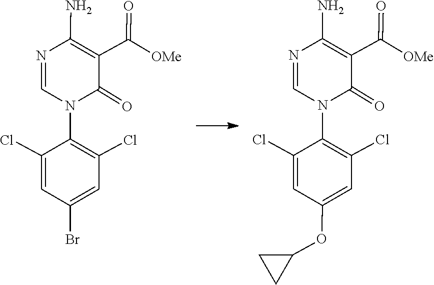 [SMILES: COC(=O)c1c(N)ncn(-c2c(Cl)cc(Br)cc2Cl)c1=O ; COC(=O)c1c(N)ncn(-c2c(Cl)cc(OC3CC3)cc2Cl)c1=O]

The title compound was prepared as a white solid (140 mg, 30%) from methyl 4-amino-1-(4-bromo-2,6-dichlorophenyl)-6-oxo-1,6-dihydropyrimidine-5-carboxylate (Intermediate B6) and cyclopropanol using an analogous method to that described for Intermediate B10. LCMS m/z=370 [M+H]⁺.

### Intermediate B12. Synthesis of methyl 4-amino-1-(2,6-dichloro-4-methylphenyl)-6-oxo-1,6-dihydropyrimidine-5-carboxylate

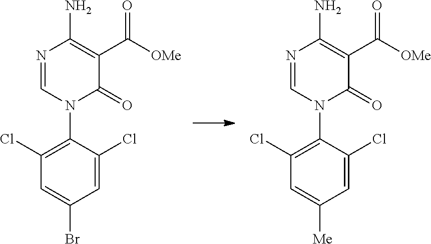 [SMILES: COC(=O)c1c(N)ncn(-c2c(Cl)cc(Br)cc2Cl)c1=O ; COC(=O)c1c(N)ncn(-c2c(Cl)cc(C)cc2Cl)c1=O]

To a solution of methyl 4-amino-1-(4-bromo-2,6-dichlorophenyl)-6-oxo-1,6-dihydropyrimidine-5-carboxylate (Intermediate B6, 500 mg, 1.27 mmol) and 2,4,6-trimethyl-1,3,5,2,4,6-trioxatriborinane (319 mg, 1.27 mmol, 355.69 μL, 50% purity) in dioxane (10 mL) was added Pd(dppf)Cl₂ (93 mg, 0.127 mmol) and K₂CO₃ (352 mg, 2.54 mmol) and the mixture stirred at 100° C. for 2 h under N₂. The reaction mixture was filtered and the filtrate was concentrated under reduced pressure and the residue purified by prep-HPLC (15-50% MeCN/H₂O (0.05% NH₄OH+10 mM NH₄HCO₃)) to give the title compound as a white solid (190 mg, 45%). LCMS m/z=328 [M+H]⁺.

### Intermediate B13. methyl 4-amino-1-((S)-2-chloro-4-cyano-6-methylphenyl)-6-oxo-1,6-dihydropyrimidine-5-carboxylate

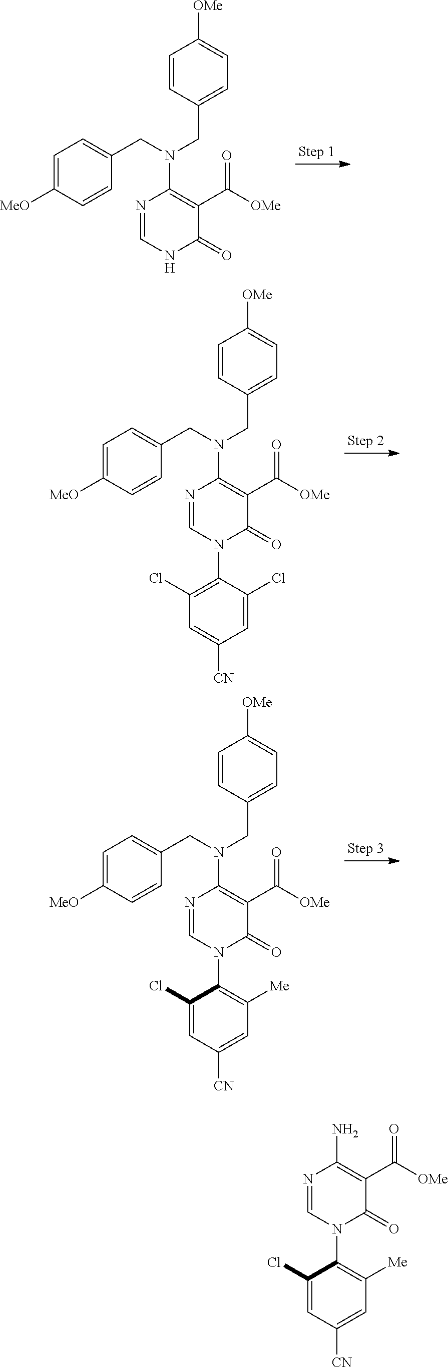

### Step 1. Synthesis of methyl 4-(bis(4-methoxybenzyl)amino)-1-(2,6-dichloro-4-cyanophenyl)-6-oxo-1,6-dihydropyrimidine-5-carboxylate

The title compound was prepared as a yellow solid (10 g, 72%) from methyl 4-(bis(4-methoxybenzyl)amino)-6-oxo-1,6-dihydropyrimidine-5-carboxylate (Intermediate B1) and 3,5-dichloro-4-fluoro-benzonitrile using an analogous method to that described for Intermediate B6, Step 1. LCMS m/z=579 [M+H]⁺.

### Step 2. Synthesis of methyl 4-(bis(4-methoxybenzyl)amino)-1-((S)-2-chloro-4-cyano-6-methylphenyl)-6-oxo-1,6-dihydropyrimidine-5-carboxylate

To a solution of methyl 4-(bis(4-methoxybenzyl)amino)-1-(2,6-dichloro-4-cyanophenyl)-6-oxo-1,6-dihydropyrimidine-5-carboxylate (5 g, 8.63 mmol) and 2,4,6-trimethyl-1,3,5,2,4,6-trioxatriborinane (2.17 g, 8.63 mmol, 2.41 mL, 50% purity) in dioxane (20 mL) was added XPHOS-PD-G2 (679 mg, 0.863 mmol) and K₃PO₄ (3.66 g, 17.3 mmol) and the mixture stirred at 80° C. for 3 h under N₂. The solids were removed by filtration and the filtrate was concentrated under reduced pressure and the residue was purified by prep-HPLC (Phenomenex Luna C18, 250×70 mm, 15 μm; 60-85% MeCN/(H₂O+0.2% HCO₂H)) to give methyl 4-(bis(2,4-dimethoxybenzyl)amino)-1-(2-chloro-4-cyano-6-methylphenyl)-6-oxo-1,6-dihydropyrimidine-5-carboxylate as a white solid (110 mg, 0.204 mmol, 2.4%). The racemate was separated by chiral SFC (DAICEL CHIRALPAK AD, 250×30 mm, 10 μm; 35% (0.1% NH₄OH/IPA) in CO₂ to afford:

Peak 1, Intermediate B13: methyl 4-(bis(4-methoxybenzyl)amino)-1-((S)-2-chloro-4-cyano-6-methylphenyl)-6-oxo-1,6-dihydropyrimidine-5-carboxylate (white solid, 320 mg). LCMS m/z=559 [M+H]⁺.

### Step 3. Synthesis of methyl 4-amino-1-((S)-2-chloro-4-cyano-6-methylphenyl)-6-oxo-1,6-dihydropyrimidine-5-carboxylate

A mixture of methyl 4-(bis(4-methoxybenzyl)amino)-1-((S)-2-chloro-4-cyano-6-methylphenyl)-6-oxo-1,6-dihydropyrimidine-5-carboxylate (Step 2, Peak 1, 320 mg, 0.572 mmol) in TFA (2 mL) was stirred at 80° C. for 15 min. The reaction mixture was filtered and the filtrate evaporated to dryness in vacuo. The residue was purified by prep-TLC (EtOAc) to give the title compound as a pale yellow solid (120 mg, 66%). LCMS m/z=319 [M+H]⁺.

### Intermediate B14. Synthesis of methyl 4-amino-1-(2,6-dichloro-4-cyanophenyl)-6-oxo-1,6-dihydropyrimidine-5-carboxylate

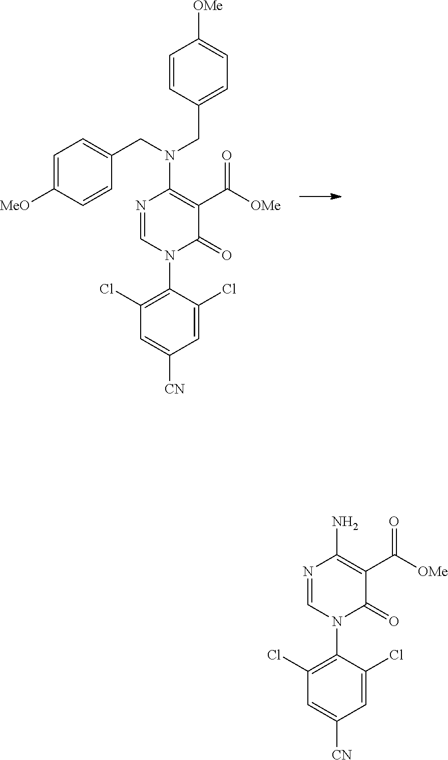 [SMILES: COC(=O)c1c(N(Cc2ccc(OC)cc2)Cc2ccc(OC)cc2)ncn(-c2c(Cl)cc(C#N)cc2Cl)c1=O ; COC(=O)c1c(N)ncn(-c2c(Cl)cc(C#N)cc2Cl)c1=O]

A solution of methyl 4-(bis(4-methoxybenzyl)amino)-1-(2,6-dichloro-4-cyanophenyl)-6-oxo-1,6-dihydropyrimidine-5-carboxylate (Intermediate B13, Step 1; 3.00 g, 5.18 mmol) in TFA (20 mL) was stirred at 80° C. for 1 hr. The reaction mixture was concentrated under reduced pressure and the residue diluted with H₂O (10 mL) and extracted with EtOAc (3×20 mL). The combined organics were washed with brine (2×15 mL), dried (Na₂SO₄) and evaporated to dryness in vacuo. The residue was purified by MPLC (SiO₂, 0-50% EtOAc/PE) to afford the title compound as white solid (1.7 g, 97%). LCMS m/z=339 [M+H]⁺.

### Intermediate B15. methyl 4-amino-6-oxo-1-(2,4,6-trichlorophenyl)-1,6-dihydropyrimidine-5-carboxylate

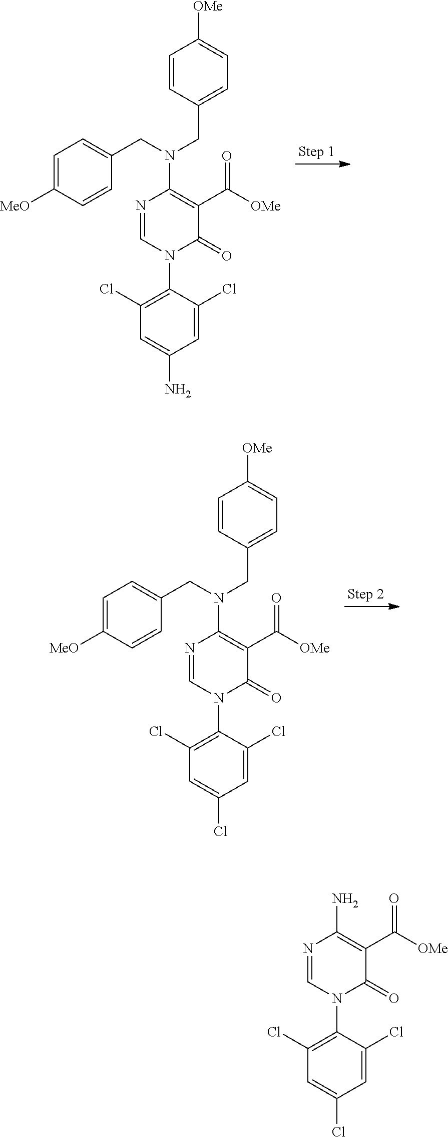 [SMILES: COC(=O)c1c(N(Cc2ccc(OC)cc2)Cc2ccc(OC)cc2)ncn(-c2c(Cl)cc(N)cc2Cl)c1=O ; COC(=O)c1c(N(Cc2ccc(OC)cc2)Cc2ccc(OC)cc2)ncn(-c2c(Cl)cc(Cl)cc2Cl)c1=O ; COC(=O)c1c(N)ncn(-c2c(Cl)cc(Cl)cc2Cl)c1=O]

### Step 1. Synthesis of methyl 4-(bis(4-methoxybenzyl)amino)-6-oxo-1-(2,4,6-trichlorophenyl)-1,6-dihydropyrimidine-5-carboxylate

To a solution of methyl 1-(4-amino-2,6-dichlorophenyl)-4-(bis(4-methoxybenzyl)amino)-6-oxo-1,6-dihydropyrimidine-5-carboxylate (Intermediate B6, Step 2; 500 mg, 0.878 mmol), CuCl (174 mg, 1.76 mmol), CuCl₂ (354 mg, 2.63 mmol) in MeCN (9 mL) was added isopentyl nitrite (308 mg, 2.63 mmol) in MeCN (0.9 mL) and the mixture stirred at 25° C. for 1 h under N₂. The reaction mixture was quenched by addition H₂O (30 mL) at 0° C. and extracted with MeCN (3×30 mL). The combined organics were dried (Na₂SO₄) and evaporated to dryness in vacuo to give the title compound as a brown oil (400 mg, crude). LCMS m/z=589 [M+H]⁺.

### Step 2. Synthesis of methyl 4-amino-6-oxo-1-(2,4,6-trichlorophenyl)-1,6-dihydropyrimidine-5-carboxylate

A mixture of methyl 4-(bis(4-methoxybenzyl)amino)-6-oxo-1-(2,4,6-trichlorophenyl)-1,6-dihydropyrimidine-5-carboxylate (350 mg, 0.594 mmol) in TFA (8 mL) was stirred at 80° C. for 1 h. The reaction mixture was concentrated under reduced pressure and the pH adjusted with 1 M Na₂CO₃ to pH 6-7. The residue was purified by column chromatography (SiO₂, 0-50% EtO Ac/PE) to give the title compound as a yellow solid (150 mg, 72%). LCMS m/z=352 [M+H]⁺.

### Intermediate B16. methyl 4-amino-1-(2,6-dichloro-4-(difluoromethyl)phenyl)-6-oxo-1,6-dihydropyrimidine-5-carboxylate

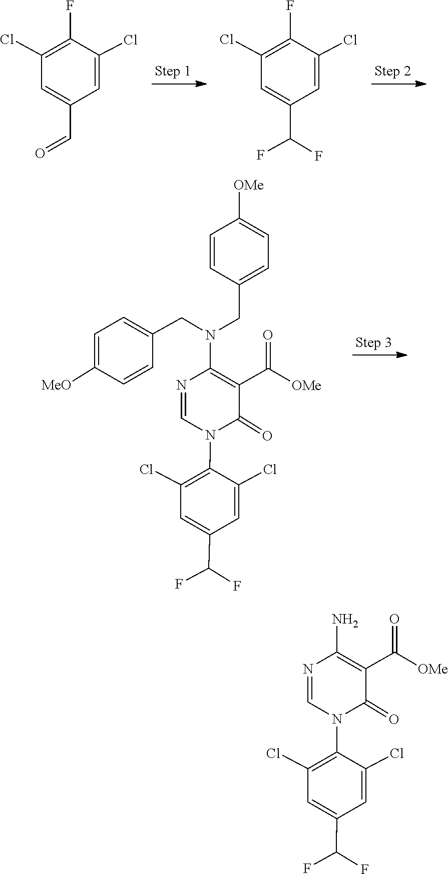

### Step 1. Synthesis of 1,3-dichloro-5-(difluoromethyl)-2-fluorobenzene

To a solution of 3,5-dichloro-4-fluoro-benzaldehyde (5 g, 25.9 mmol) in DCM (50 mL) was added DAST (20.88 g, 130 mmol) at −78° C. The mixture was stirred at 25° C. for 3 h under N₂. The reaction mixture was quenched by addition saturated aqueous NaHCO₃ (30 mL) at 0° C. and extracted with DCM (3×20 mL). The combined organics were dried (Na₂SO₄) and evaporated to dryness in vacuo. The residue was purified by column chromatography (SiO₂, 20:1 PE/EtOAc) to give the title compound as a light yellow oil (5.3 g, 95%) which was used without further purification.

### Step 2. Synthesis of methyl 4-(bis(4-methoxybenzyl)amino)-1-(2,6-dichloro-4-(difluoromethyl)phenyl)-6-oxo-1,6-dihydropyrimidine-5-carboxylate

To a solution of 1,3-dichloro-5-(difluoromethyl)-2-fluorobenzene (2.7 g, 12.6 mmol) and methyl 4-(bis(4-methoxybenzyl)amino)-6-oxo-1,6-dihydropyrimidine-5-carboxylate (Intermediate B1, 2.86 g, 6.98 mmol) in DMF (20 mL) was added K₂CO₃ (1.93 g, 13.95 mmol) and the mixture was stirred at 80° C. for 2 h. The reaction mixture was filtered and the filtrate concentrated under reduced pressure to give a residue which was purified by column chromatography (SiO₂, 0-50% EtOAc/PE) to give the title compound as a light yellow solid (2.24 g, 53%). LCMS m/z=604 [M+H]⁺.

### Step 3. Synthesis of methyl 4-amino-1-(2,6-dichloro-4-(difluoromethyl)phenyl)-6-oxo-1,6-dihydropyrimidine-5-carboxylate

A mixture of methyl 4-(bis(4-methoxybenzyl)amino)-1-(2,6-dichloro-4-(difluoromethyl)phenyl)-6-oxo-1,6-dihydropyrimidine-5-carboxylate (2.24 g, 3.71 mmol) in TFA (10 mL) was stirred at 80° C. for 1 hr. The reaction mixture was filtered and the filtrate was concentrated under reduced pressure to give a residue which was purified by column chromatography (SiO₂, 0-50% EtOAc/PE) to give the title compound as a light yellow solid (1.35 g, 100%). LCMS m/z=364 [M+H]⁺.

### Intermediate B17. Synthesis of methyl 4-amino-1-(4-(difluoromethyl)-2,6-dimethylphenyl)-6-oxo-1,6-dihydropyrimidine-5-carboxylate

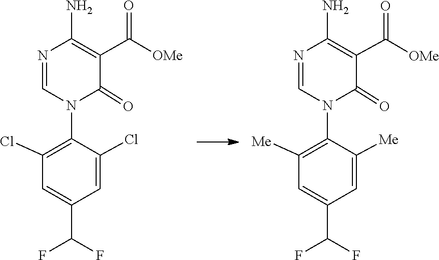 [SMILES: COC(=O)c1c(N)ncn(-c2c(Cl)cc(C(F)F)cc2Cl)c1=O ; COC(=O)c1c(N)ncn(-c2c(C)cc(C(F)F)cc2C)c1=O]

To the mixture of methyl 4-amino-1-(2,6-dichloro-4-(difluoromethyl)phenyl)-6-oxo-1,6-dihydropyrimidine-5-carboxylate (Intermediate B16, 500 mg, 1.37 mmol) in dioxane (9 mL) was added 2,4,6-trimethyl-1,3,5,2,4,6-trioxatriborinane (1.03 g, 4.12 mmol, 1.15 mL, 50% purity) and Xphos-Pd-G2 (108 mg, 0.137 mmol) and K₃PO₄ (583 mg, 2.75 mmol) was degassed and purged with N₂ (×3) and the mixture stirred at 80° C. for 12 h under N₂. The reaction mixture was filtered and the filtrate was concentrated under reduced pressure and the residue purified by column chromatography on silica gel (0-50% EtOAc/PE) to give the title compound as a yellow oil (800 mg, 90%). LCMS m/z=324 [M+H]⁺.

### Intermediate B18. methyl 4-amino-1-(4-methoxy-2,6-dimethylphenyl)-6-oxo-1,6-dihydropyrimidine-5-carboxylate

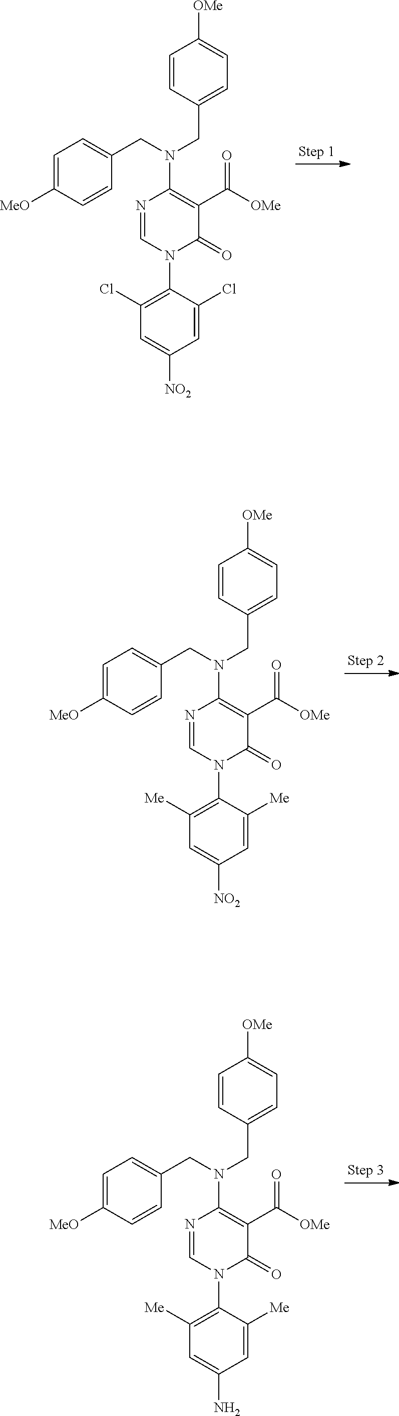 [SMILES: COC(=O)c1c(N(Cc2ccc(OC)cc2)Cc2ccc(OC)cc2)ncn(-c2c(Cl)cc([N+](=O)[O-])cc2Cl)c1=O ; COC(=O)c1c(N(Cc2ccc(OC)cc2)Cc2ccc(OC)cc2)ncn(-c2c(C)cc([N+](=O)[O-])cc2C)c1=O ; COC(=O)c1c(N(Cc2ccc(OC)cc2)Cc2ccc(OC)cc2)ncn(-c2c(C)cc(N)cc2C)c1=O]

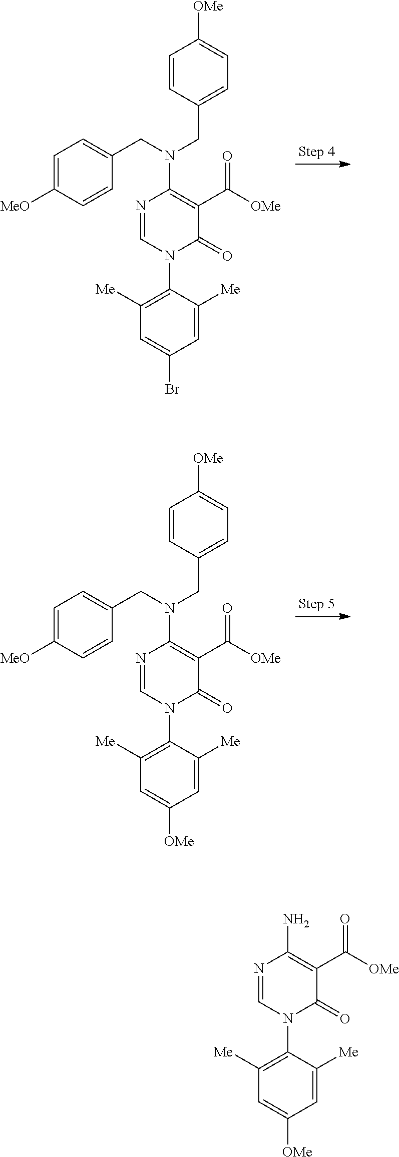 [SMILES: COC(=O)c1c(N(Cc2ccc(OC)cc2)Cc2ccc(OC)cc2)ncn(-c2c(C)cc(Br)cc2C)c1=O ; COC(=O)c1c(N(Cc2ccc(OC)cc2)Cc2ccc(OC)cc2)ncn(-c2c(C)cc(OC)cc2C)c1=O ; COC(=O)c1c(N)ncn(-c2c(C)cc(OC)cc2C)c1=O]

### Step 1. Synthesis of methyl 4-(bis(4-methoxybenzyl)amino)-1-(2,6-dimethyl-4-nitrophenyl)-6-oxo-1,6-dihydropyrimidine-5-carboxylate

To a mixture of methyl 4-(bis(4-methoxybenzyl)amino)-1-(2,6-dichloro-4-nitrophenyl)-6-oxo-1,6-dihydropyrimidine-5-carboxylate (Intermediate B6 Step 1, 6.00 g, 10.01 mmol) and 2,4,6-trimethyl-1,3,5,2,4,6-trioxatriborinane (5.03 g, 20.02 mmol) in dioxane (100 mL) was added K₃PO₄ (4.25 g, 20.02 mmol) and Xphos-Pd-G2 (472.54 mg, 0.60 mmol) and the mixture stirred at 80° C. for 12 h under N₂. The mixture was filtered and concentrated under reduced pressure and the residue purified by column chromatography (SiO₂, 25-50% EtOAc/PE) to give the title compound as a yellow solid (5.00 g, 89%). LCMS m/z=559 [M+H]⁺.

### Step 2. Synthesis of methyl 1-(4-amino-2,6-dimethylphenyl)-4-(bis(4-methoxybenzyl)amino)-6-oxo-1,6-dihydropyrimidine-5-carboxylate

To a mixture of methyl 4-(bis(4-methoxybenzyl)amino)-1-(2,6-dimethyl-4-nitrophenyl)-6-oxo-1,6-dihydropyrimidine-5-carboxylate (Step 1, 5.00 g, 8.95 mmol) in THF (50 mL) and MeOH (50 mL) was added Pd/C (6.00 g, 10% purity) and the mixture stirred at 25° C. for 2 h under H₂ (15 psi). The mixture was filtered and the filtrate concentrated under reduced pressure and the residue purified by prep-HPLC-23 (50-70% MeCN) to give the title compound as a yellow solid (4.50 g, crude). LCMS m/z=529 [M+H]⁺.

### Step 3. Synthesis of methyl 4-(bis(4-methoxybenzyl)amino)-1-(4-bromo-2,6-dimethylphenyl)-6-oxo-1,6-dihydropyrimidine-5-carboxylate

To a mixture of methyl 1-(4-amino-2,6-dimethylphenyl)-4-(bis(4-methoxybenzyl)amino)-6-oxo-1,6-dihydropyrimidine-5-carboxylate (Step 2, 1.92 g, 3.63 mmol, 1.00 eq), CuBr (625 mg, 4.36 mmol), CSA (2.53 g, 10.9 mmol) and TBAB (7.03 g, 21.8 mmol) in MeCN (30 mL) was added NaNO₂ (752 mg, 10.9 mmol) and the mixture stirred at 25° C. for 1 h. The mixture was concentrated under reduced pressure and the residue purified by column chromatography (SiO₂, 0-100% EtOAc/PE) to give the title compound as a white solid (1.70 g, 79%). LCMS m/z=592 [M+H]⁺.

### Step 4. Synthesis of methyl 4-(bis(4-methoxybenzyl)amino)-1-(4-methoxy-2,6-dimethylphenyl)-6-oxo-1,6-dihydropyrimidine-5-carboxylate

A mixture of methyl 4-(bis(4-methoxybenzyl)amino)-1-(4-bromo-2,6-dimethylphenyl)-6-oxo-1,6-dihydropyrimidine-5-carboxylate (Step 3, 1.12 g, 1.89 mmol), Pd₂ (dba)₃ (173 mg, 0.19 mmol), t-Bu Xphos (80.3 mg, 0.19 mmol) and K₃PO₄ (602 mg, 2.84 mmol) in toluene (8 mL) and MeOH (8 mL) was stirred at 80° C. for 2 h under N₂. The reaction mixture was filtered and the filtrate was concentrated under reduced pressure and the residue purified by column chromatography on silica gel (0-50% EtOAc/PE) to give the title compound as a brown oil (800 mg, 77%). LCMS m/z=544 [M+H]⁺.

### Step 5. Synthesis of methyl 4-amino-1-(4-methoxy-2,6-dimethylphenyl)-6-oxo-1,6-dihydropyrimidine-5-carboxylate

A solution of methyl 4-(bis(4-methoxybenzyl)amino)-1-(4-methoxy-2,6-dimethylphenyl)-6-oxo-1,6-dihydropyrimidine-5-carboxylate (Step 4, 1.20 g, 2.21 mmol) in TFA (20 mL) was stirred at 80° C. for 1 h. The reaction mixture was concentrated under reduced pressure and the pH adjusted with 1 M Na₂CO₃ to pH 6-7 and the residue purified by column chromatography (0-50% EtOAc/PE) to give the title compound as a pale yellow solid (560 mg, 83%). LCMS m/z=304 [M+H]⁺.

### Intermediate B19. methyl 4-amino-1-(2,6-dichloro-4-(methoxymethyl)phenyl)-6-oxo-1,6-dihydropyrimidine-5-carboxylate

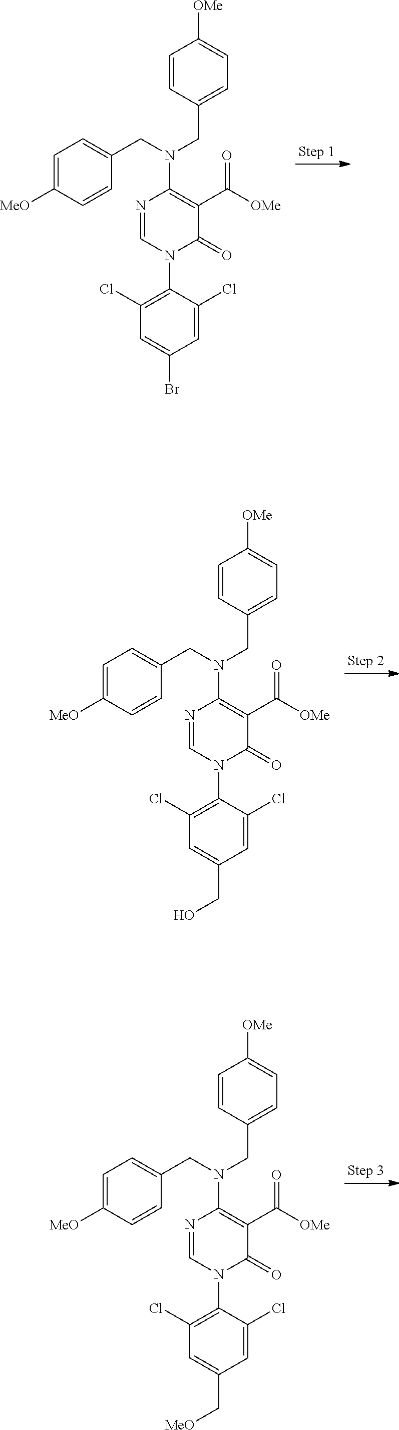 [SMILES: COC(=O)c1c(N(Cc2ccc(OC)cc2)Cc2ccc(OC)cc2)ncn(-c2c(Cl)cc(Br)cc2Cl)c1=O ; COC(=O)c1c(N(Cc2ccc(OC)cc2)Cc2ccc(OC)cc2)ncn(-c2c(Cl)cc(CO)cc2Cl)c1=O ; COCc1cc(Cl)c(-n2cnc(N(Cc3ccc(OC)cc3)Cc3ccc(OC)cc3)c(C(=O)OC)c2=O)c(Cl)c1]

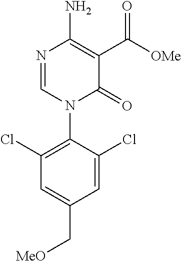 [SMILES: COCc1cc(Cl)c(-n2cnc(N)c(C(=O)OC)c2=O)c(Cl)c1]

### Step 1. Synthesis of methyl 4-(bis(4-methoxybenzyl)amino)-1-(2,6-dichloro-4-(hydroxymethyl)phenyl)-6-oxo-1,6-dihydropyrimidine-5-carboxylate

To a solution of tributylstannylmethanol (1.67 g, 5.21 mmol), methyl 4-(bis(4-methoxybenzyl)amino)-1-(4-bromo-2,6-dichlorophenyl)-6-oxo-1,6-dihydropyrimidine-5-carboxylate (Intermediate B6 Step 3; 1.10 g, 1.74 mmol) in dioxane (10 mL) was added Pd(PPh₃)₄ (100 mg, 0.087 mmol) and the mixture was stirred at 100° C. for 3 h under N₂. The residue was purified by MPLC (SiO₂, 0-50% EtOAc/PE) to give the title compound as a yellow solid (2.60 g, 85%). LCMS m/z=584 [M+H]⁺.

### Step 2. Synthesis of methyl 4-(bis(4-methoxybenzyl)amino)-1-(2,6-dichloro-4-(methoxymethyl)phenyl)-6-oxo-1,6-dihydropyrimidine-5-carboxylate

To a solution of methyl 4-(bis(4-methoxybenzyl)amino)-1-(2,6-dichloro-4-(hydroxymethyl)phenyl)-6-oxo-1,6-dihydropyrimidine-5-carboxylate (2.15 g, 3.68 mmol) in THF (25 mL) was added NaOH (220 mg, 5.52 mmol) and dimethyl sulfate (4.07 g, 32.27 mmol) and the mixture was stirred at 25° C. for 12 h. The reaction mixture was diluted with H₂O (50 mL) and extracted with EtOAc (3×25 mL), dried (Na₂SO₄) and concentrated under reduced pressure. The residue was purified by prep-HPLC-11 (35-70% MeCN) to give the title compound as a white oil (180 mg, 0.300 mmol, 8.2%). LCMS m/z=598 [M+H]⁺.

### Step 3. Synthesis of methyl 4-amino-1-(2,6-dichloro-4-(methoxymethyl)phenyl)-6-oxo-1,6-dihydropyrimidine-5-carboxylate

A mixture of methyl 4-(bis(2,4-dimethoxybenzyl)amino)-1-(2,6-dichloro-4-(methoxymethyl)phenyl)-6-oxo-1,6-dihydropyrimidine-5-carboxylate (180 mg, 0.300 mol) in TFA (2 mL) was stirred at 80° C. for 0.5 h. The residue was purified by prep-TLC (50% EtOAc/PE) to give the title compound as a brown solid (60 mg, 0.168 mmol, 55.7%). LCMS m/z=358 [M+H]⁺.

### Intermediate B20. Synthesis of tert-butyl ((5-(4-amino-1-(4-bromo-2,6-dichlorophenyl)-6-oxo-1,6-dihydropyrimidine-5-carboxamido)pyridin-3-yl)methyl)(ethyl)carbamate

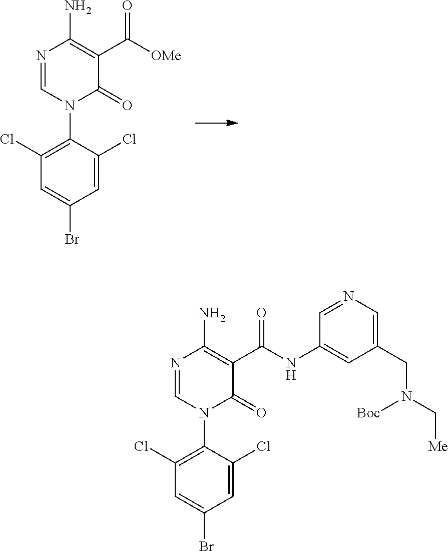 [SMILES: COC(=O)c1c(N)ncn(-c2c(Cl)cc(Br)cc2Cl)c1=O ; CCN(Cc1cncc(NC(=O)c2c(N)ncn(-c3c(Cl)cc(Br)cc3Cl)c2=O)c1)C(=O)OC(C)(C)C]

To a mixture of methyl 4-amino-1-(4-bromo-2,6-dichloro-phenyl)-6-oxo-pyrimidine-5-carboxylate (Intermediate B6, 500 mg, 1.27 mmol) and tert-butyl ((5-aminopyridin-3-yl)methyl)(ethyl)carbamate (Intermediate A24, 479.6 mg, 1.91 mmol) in toluene (9 mL) was added AlMe₃ (2 M in toluene, 1.91 mL) at 0° C. The mixture was stirred at 100° C. for 0.5 h under N₂ atmosphere. The reaction mixture was quenched by addition of 1 M NaOH aq. (50 mL) at 0° C. and extracted with EtOAc (3×50 mL). The combined organics were dried (Na₂SO₄) and concentrated under reduced pressure to give a residue that was purified by column chromatography (SiO₂, 0-100% EtOAc/PE) to give the title compound as a yellow solid (2 g, 64%). LCMS m/z=613 [M+H]⁺.

### Intermediate B21. Synthesis of 4-(4-amino-5-((5-(((tert-butoxycarbonyl) (ethyl)amino)methyl)pyridin-3-yl)carbamoyl)-6-oxopyrimidin-1 (6H)-yl)-3,5-dichlorobenzoic acid

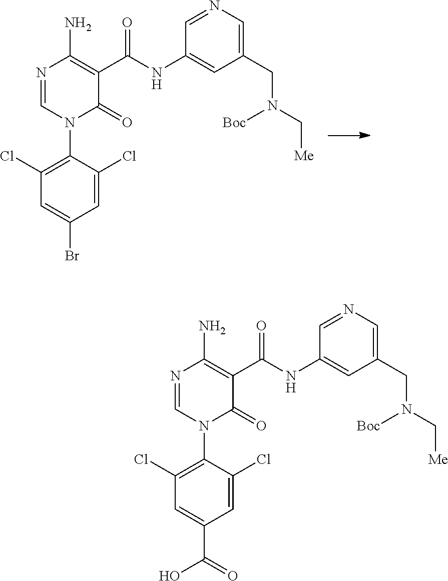 [SMILES: CCN(Cc1cncc(NC(=O)c2c(N)ncn(-c3c(Cl)cc(Br)cc3Cl)c2=O)c1)C(=O)OC(C)(C)C ; CCN(Cc1cncc(NC(=O)c2c(N)ncn(-c3c(Cl)cc(C(=O)O)cc3Cl)c2=O)c1)C(=O)OC(C)(C)C]

To a solution of tert-butyl ((5-(4-amino-1-(4-bromo-2,6-dichlorophenyl)-6-oxo-1,6-dihydropyrimidine-5-carboxamido)pyridin-3-yl)methyl)(ethyl)carbamate (310 mg, 0.506 mmol) and oxalic acid (319 mg, 3.54 mmol) in DMF (2 mL) was added Pd(OAc)₂ (11.37 mg, 0.051 mmol), Xantphos (29.29 mg, 0.051 mol), Ac₂O (155 mg, 1.52 mmol) and DIPEA (196 mg, 1.52 mmol) and the mixture stirred at 100° C. for 12 h under N₂. The reaction mixture was filtered and the filtrate concentrated under reduced pressure and the residue purified by prep-HPLC-24 to give the title compound as a white solid (120 mg, 41%). LCMS m/z=577 [M+H]⁺.

### Intermediate B22. methyl 4-amino-1-(4-(methoxymethyl)-2,6-dimethylphenyl)-6-oxo-1,6-dihydropyrimidine-5-carboxylate

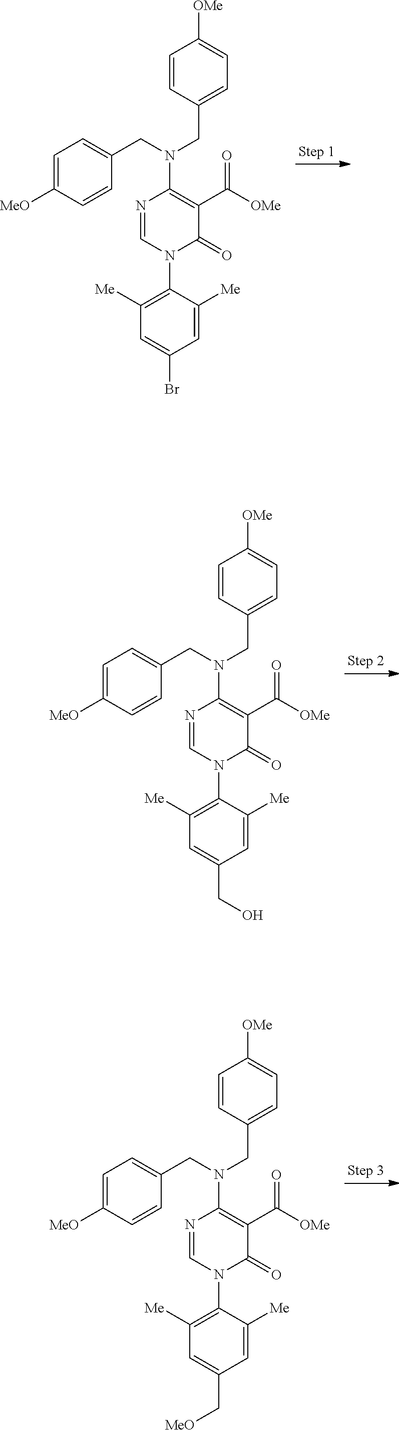 [SMILES: COC(=O)c1c(N(Cc2ccc(OC)cc2)Cc2ccc(OC)cc2)ncn(-c2c(C)cc(Br)cc2C)c1=O ; COC(=O)c1c(N(Cc2ccc(OC)cc2)Cc2ccc(OC)cc2)ncn(-c2c(C)cc(CO)cc2C)c1=O ; COCc1cc(C)c(-n2cnc(N(Cc3ccc(OC)cc3)Cc3ccc(OC)cc3)c(C(=O)OC)c2=O)c(C)c1]

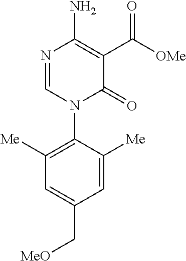 [SMILES: COCc1cc(C)c(-n2cnc(N)c(C(=O)OC)c2=O)c(C)c1]

### Step 1. Synthesis of methyl 4-(bis(4-methoxybenzyl)amino)-1-(4-(hydroxymethyl)-2,6-dimethylphenyl)-6-oxo-1,6-dihydropyrimidine-5-carboxylate

To a solution of methyl 4-(bis(4-methoxybenzyl)amino)-1-(4-bromo-2,6-dimethylphenyl)-6-oxo-1,6-dihydropyrimidine-5-carboxylate (Intermediate B18 Step 3, 750 mg, 1.27 mmol) and tributylstannylmethanol (1.22 g, 3.80 mmol) in dioxane (7 mL) was added Pd(PPh₃)₄ (146 mg, 0.127 mmol). The mixture was stirred at 100° C. for 12 h under N₂. The reaction mixture was concentrated under reduced pressure and the residue purified by MPLC (SiO₂, 0-100% EtOAc/PE) to give the title compound as a yellow oil (1.30 g, 2.39 mmol, 94%). LCMS m/z=544 [M+H]⁺.

### Step 2. Synthesis of methyl 4-(bis(4-methoxybenzyl)amino)-1-(4-(methoxymethyl)-2,6-dimethylphenyl)-6-oxo-1,6-dihydropyrimidine-5-carboxylate

To a solution of methyl 4-(bis(4-methoxybenzyl)amino)-1-(4-(hydroxymethyl)-2,6-dimethylphenyl)-6-oxo-1,6-dihydropyrimidine-5-carboxylate (200 mg, 0.368 mmol) in THF (5 mL) was added NaOH (22.1 mg, 0.552 mmol) and dimethyl sulfate (381 mg, 3.02 mmol) and the mixture stirred at 25° C. for 12 h. The reaction mixture was diluted with H₂O (35 mL) and extracted with EtOAc (3×35 mL). The combined extracts were dried (Na₂SO₄), and evaporated to dryness in vacuo. The residue was purified by column chromatography (SiO₂, 0-100% EtOAc/PE) to give the title compound as a yellow oil (300 mg, 24%). LCMS m/z=558 [M+H]⁺.

### Step 3. Synthesis of methyl 4-(bis(4-methoxybenzyl)amino)-1-(4-(methoxymethyl)-2,6-dimethylphenyl)-6-oxo-1,6-dihydropyrimidine-5-carboxylate

A solution of methyl 4-(bis(4-methoxybenzyl)amino)-1-(4-(methoxymethyl)-2,6-dimethylphenyl)-6-oxo-1,6-dihydropyrimidine-5-carboxylate (200 mg, 0.359 mmol) in TFA (5 mL) was stirred at 80° C. for 1 h. The reaction mixture was concentrated under reduced pressure and the pH of the residue adjusted with 1 M NH₄OH to pH 6-7. The residue was purified by column chromatography (SiO₂, 0-100% EtOAc/PE) to afford the title compound as a white solid (110 mg, 97%). LCMS m/z=318 [M+H].

### Intermediate B23. methyl 4-amino-1-(2,6-dichloro-4-(trifluoromethyl)phenyl)-6-oxo-1,6-dihydropyrimidine-5-carboxylate

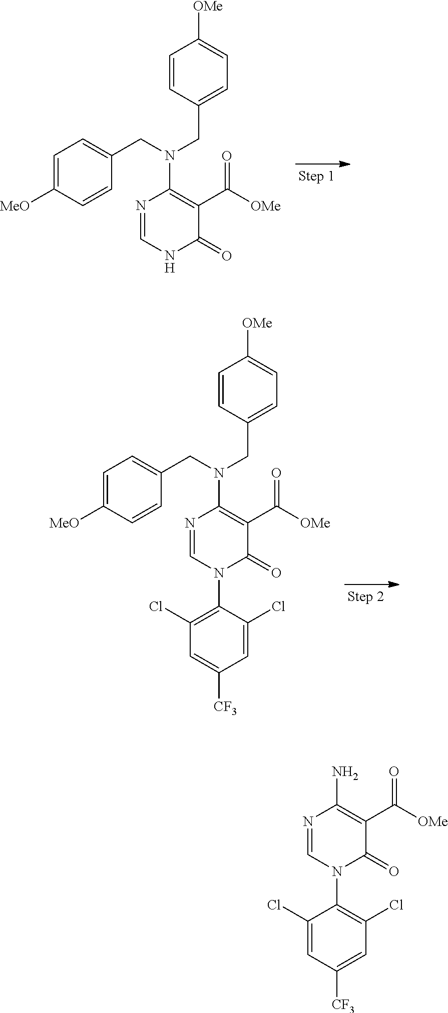 [SMILES: COC(=O)c1c(N(Cc2ccc(OC)cc2)Cc2ccc(OC)cc2)nc[nH]c1=O ; COC(=O)c1c(N(Cc2ccc(OC)cc2)Cc2ccc(OC)cc2)ncn(-c2c(Cl)cc(C(F)(F)F)cc2Cl)c1=O ; COC(=O)c1c(N)ncn(-c2c(Cl)cc(C(F)(F)F)cc2Cl)c1=O]

### Step 1. Synthesis of methyl 4-(bis(4-methoxybenzyl)amino)-1-(2,6-dichloro-4-(trifluoromethyl)phenyl)-6-oxo-1,6-dihydropyrimidine-5-carboxylate

To a mixture of methyl 4-(bis(4-methoxybenzyl)amino)-6-oxo-1,6-dihydropyrimidine-5-carboxylate (Intermediate B1, 5 g, 12.21 mmol) and 1,3-dichloro-2-fluoro-5-(trifluoromethyl)benzene (3.19 g, 13.43 mmol) and K₂CO₃ (3.38 g, 24.42 mmol) in DMF (15 mL) was stirred at 80° C. for 3 h. The reaction mixture was filtered and H₂O added to the filtrate. Then resulting solids were collected by filtration to afford the title compound as a yellow solid (7.50 g, crude) which was used without further purification. LCMS m/z=623 [M+H]⁺.

### Step 2. Synthesis of methyl 4-amino-1-(2,6-dichloro-4-(trifluoromethyl)phenyl)-6-oxo-1,6-dihydropyrimidine-5-carboxylate

A mixture of methyl 4-(bis(4-methoxybenzyl)amino)-1-(2,6-dichloro-4-(trifluoromethyl)phenyl)-6-oxo-1,6-dihydropyrimidine-5-carboxylate (7.50 g, 12.05 mmol) in TFA (20 mL) was stirred at 80° C. for 1 h. The reaction mixture was concentrated under reduced pressure and the pH of the residue adjusted to pH 6-7 with 1M NH₄OH and extracted with EtOAc. The combined organics were dried (Na₂SO₄) and concentrated under reduced pressure. The residue was purified by column chromatography (SiO₂, 0-50% EtOAc/PE) to give the title compound as a white solid (3.5 g, 76%) as. LCMS m/z=382 [M+H]⁺.

### Intermediate B24. methyl 4-amino-1-(2,6-dimethyl-4-(trifluoromethyl)phenyl)-6-oxo-1,6-dihydropyrimidine-5-carboxylate

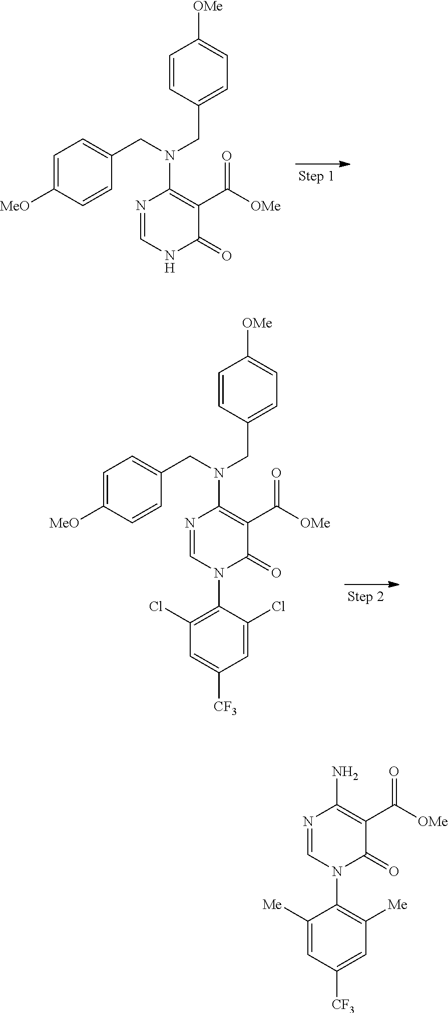 [SMILES: COC(=O)c1c(N(Cc2ccc(OC)cc2)Cc2ccc(OC)cc2)nc[nH]c1=O ; COC(=O)c1c(N(Cc2ccc(OC)cc2)Cc2ccc(OC)cc2)ncn(-c2c(Cl)cc(C(F)(F)F)cc2Cl)c1=O ; COC(=O)c1c(N)ncn(-c2c(C)cc(C(F)(F)F)cc2C)c1=O]

### Step 1. Synthesis of methyl 4-(bis(4-methoxybenzyl)amino)-1-(2,6-dichloro-4-(trifluoromethyl)phenyl)-6-oxo-1,6-dihydropyrimidine-5-carboxylate

A suspension of methyl 4-(bis(4-methoxybenzyl)amino)-6-oxo-1,6-dihydropyrimidine-5-carboxylate (Intermediate B1, 1.605 g, 4.0 mmol), K₂CO₃ (1.129 g, 8.0 mmol, 2.0 equiv) and 1,3-dichloro-2-fluoro-5-(trifluoromethyl)benzene (0.650 mL, 4.20 mmol) in DMA (8 mL) was heated to 80° C. for 22 h. The reaction was cooled, diluted with EtOAc (75 mL) and washed three times with water. The aqueous phase was back-extracted with EtOAc (20 mL). The combined organics were washed with brine, dried (MgSO₄) and concentrated by rotary evaporation. Flash chromatography (ISCO 40 g silica, 0-100% EtOAc/hex) provided the title compound as a light yellow foam (1.690 g, 68%). LCMS m/z=622 [M+H]⁺.

### Step 2. Synthesis of methyl 4-amino-1-(2,6-dimethyl-4-(trifluoromethyl)phenyl)-6-oxo-1,6-dihydropyrimidine-5-carboxylate

To a suspension of methyl 4-(bis(4-methoxybenzyl)amino)-1-(2,6-dichloro-4-(trifluoromethyl)phenyl)-6-oxo-1,6-dihydropyrimidine-5-carboxylate (Step 1, 1.681 g, 2.70 mmol), K₃PO₄ (1.72 g, 8.10 mmol), and Xphos Pd G3 (240 mg, 0.270 mmol) in dioxane (9 mL) was added trimethylboroxine solution (50 wt % in THF, 1.80 mL, 6.75 mmol). The solution was purged with N₂ for 8 min and then heated to 80° C. for 16 h. The reaction was then cooled, filtered through Celite, washed with EtOAc. Flash chromatography (ISCO 40g silica, 0-100% EtOAc/hex) provided a light yellow solid. The intermediate methyl 4-(bis(4-methoxybenzyl)amino)-1-(2,6-dimethyl-4-(trifluoromethyl)phenyl)-6-oxo-1,6-dihydropyrimidine-5-carboxylate was dissolved in TFA (8 mL) and heated to 80° C. for 2 h. The reaction was poured into saturated aqueous NaHCO₃ and extracted with EtOAc (3×40 mL). The combined organics were washed with water, brine, dried (MgSO₄) and concentrated by rotary evaporation. Flash chromatography (ISCO 24 g silica, 0-100% EtOAc/hex) provided the title compound as a peach-coloured solid (779 mg, 85% yield over 2 steps). LCMS m/z=342 [M+H]⁺.

### Intermediate B25. methyl 4-amino-1-(4-ethoxy-2,6-dimethylphenyl)-6-oxo-1,6-dihydropyrimidine-5-carboxylate

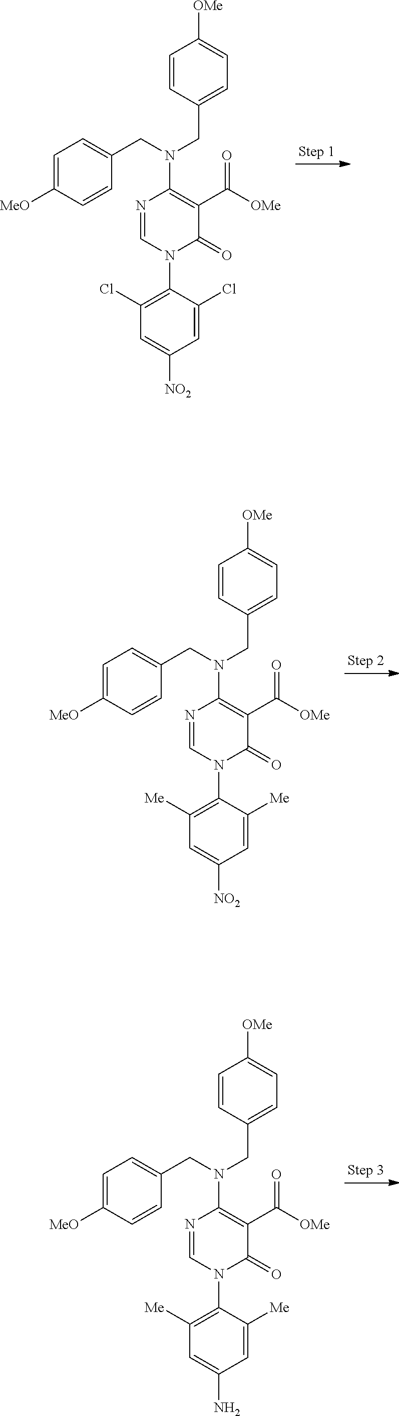 [SMILES: COC(=O)c1c(N(Cc2ccc(OC)cc2)Cc2ccc(OC)cc2)ncn(-c2c(Cl)cc([N+](=O)[O-])cc2Cl)c1=O ; COC(=O)c1c(N(Cc2ccc(OC)cc2)Cc2ccc(OC)cc2)ncn(-c2c(C)cc([N+](=O)[O-])cc2C)c1=O ; COC(=O)c1c(N(Cc2ccc(OC)cc2)Cc2ccc(OC)cc2)ncn(-c2c(C)cc(N)cc2C)c1=O]

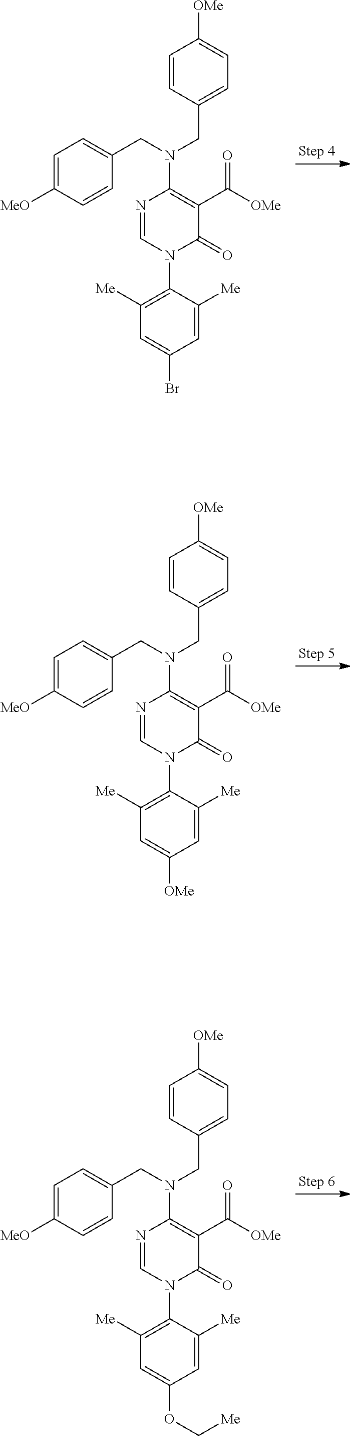 [SMILES: COC(=O)c1c(N(Cc2ccc(OC)cc2)Cc2ccc(OC)cc2)ncn(-c2c(C)cc(Br)cc2C)c1=O ; COC(=O)c1c(N(Cc2ccc(OC)cc2)Cc2ccc(OC)cc2)ncn(-c2c(C)cc(OC)cc2C)c1=O ; CCOc1cc(C)c(-n2cnc(N(Cc3ccc(OC)cc3)Cc3ccc(OC)cc3)c(C(=O)OC)c2=O)c(C)c1]

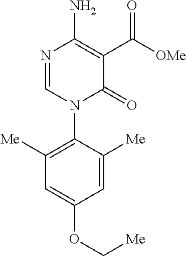 [SMILES: CCOc1cc(C)c(-n2cnc(N)c(C(=O)OC)c2=O)c(C)c1]

### Step 1. Synthesis of methyl 4-(bis(4-methoxybenzyl)amino)-1-(2,6-dimethyl-4-nitrophenyl)-6-oxo-1,6-dihydropyrimidine-5-carboxylate

K₃PO₄ (4.60 g, 21.69 mmol) and XPHOS-PD-G2 (512 mg, 0.651 mmol) were added to a mixture of methyl 4-(bis(4-methoxybenzyl)amino)-1-(2,6-dichloro-4-nitrophenyl)-6-oxo-1,6-dihydropyrimidine-5-carboxylate (Intermediate B6 Step 1, 6.5 g, 10.84 mmol) and 2,4,6-trimethyl-1,3,5,2,4,6-trioxatriborinane (5.45 g, 21.69 mmol) in dioxane (100 mL) and the mixture stirred at 80° C. for 12 h under N₂. The mixture was filtered and the filtrate concentrated under reduced pressure and the residue purified by MPLC (SiO₂, 0-50% EtOAc/PE) to give the title compound as a yellow solid (5 g, crude). LCMS m/z=559 [M+H]⁺.

### Step 2. Synthesis of methyl 1-(4-amino-2,6-dimethylphenyl)-4-(bis(4-methoxybenzyl)amino)-6-oxo-1,6-dihydropyrimidine-5-carboxylate

To a mixture of methyl 4-(bis(4-methoxybenzyl)amino)-1-(2,6-dimethyl-4-nitrophenyl)-6-oxo-1,6-dihydropyrimidine-5-carboxylate (4.7 g, 8.41 mmol) in THF (30 mL) and MeOH (30 mL) was added Pd/C (0.5 g, 8.41 mmol, 10% purity) and the mixture stirred at 20° C. for 2 h under H₂ (15 psi). The mixture was filtered and the filtrate concentrated under reduced pressure to give the title compound as a yellow solid (4.5 g, crude). LCMS m/z=529 [M+H]⁺.

### Step 3. Synthesis of methyl 4-(bis(4-methoxybenzyl)amino)-1-(4-bromo-2,6-dimethylphenyl)-6-oxo-1,6-dihydropyrimidine-5-carboxylate

NaNO₂ (1.64 g, 23.84 mmol) was added to a mixture of methyl 1-(4-amino-2,6-dimethylphenyl)-4-(bis(4-methoxybenzyl)amino)-6-oxo-1,6-dihydropyrimidine-5-carboxylate (Step 2, 4.2 g, 7.95 mmol), CuBr (1.37 g, 9.53 mmol), TBAB (15.37 g, 47.67 mmol) and CSA (5.54 g, 23.84 mmol) in MeCN (60 mL) and the mixture stirred at 20° C. for 3 h. The mixture was concentrated under reduced pressure and the residue was partitioned between EtOAc (20 mL) and H₂O (15 mL). The aqueous phase was extracted with EtOAc (3×20 mL) and the combined organics dried (Na₂SO₄) and evaporated to dryness in vacuo. The residue was purified by MPLC (SiO₂, 0-50% EtOAc/PE) to give the title compound as a yellow oil (3.6 g, 76.5%). LCMS m/z=592 [M+H]⁺.

### Step 4. Synthesis of methyl 4-(bis(4-methoxybenzyl)amino)-1-(4-hydroxy-2,6-dimethylphenyl)-6-oxo-1,6-dihydropyrimidine-5-carboxylate

To a solution of methyl 4-(bis(4-methoxybenzyl)amino)-1-(4-bromo-2,6-dimethylphenyl)-6-oxo-1,6-dihydropyrimidine-5-carboxylate (B18 Step 3, 710 mg, 1.20 mmol) in dioxane (7.5 mL) and H₂O (2.5 mL) was added K₃PO₄ (382 mg, 1.80 mmol), t-Bu Xphos (50.9 mg, 0.120 mmol) and Pd₂ (dba)₃ (110 mg, 0.120 mmol) and the mixture stirred at 80° C. for 1 h under N₂. The reaction mixture was filtered and the filtrate was concentrated under reduced pressure to give a residue that was purified by column chromatography (SiO₂, 0-33% EtOAc/PE) to give the title compound as a brown oil (1.6 g, 84%). LCMS m/z=530 [M+H]⁺.

### Step 5. Synthesis of methyl 4-(bis(4-methoxybenzyl)amino)-1-(4-ethoxy-2,6-dimethylphenyl)-6-oxo-1,6-dihydropyrimidine-5-carboxylate

To a solution of methyl 4-(bis(4-methoxybenzyl)amino)-1-(4-hydroxy-2,6-dimethylphenyl)-6-oxo-1,6-dihydropyrimidine-5-carboxylate (1 g, 1.89 mmol) and iodoethane (353.4 mg, 2.27 mmol) in DMF (5 mL) was added K₂CO₃ (522 mg, 3.78 mmol) and the mixture stirred at 60° C. for 2 h. The reaction mixture was diluted with H₂O (20 mL) and extracted with EtOAc (3×10 mL). The combined organics were dried (Na₂SO₄) and concentrated under reduced pressure to give the title compound as a brown oil (1 g, crude). LCMS m/z=558 [M+H]⁺.

### Step 6. Synthesis of methyl 4-amino-1-(4-ethoxy-2,6-dimethylphenyl)-6-oxo-1,6-dihydropyrimidine-5-carboxylate

A solution of methyl 4-(bis(4-methoxybenzyl)amino)-1-(4-ethoxy-2,6-dimethylphenyl)-6-oxo-1,6-dihydropyrimidine-5-carboxylate (1 g, 1.79 mmol) in TFA (1 mL) was stirred at 80° C. for 1 h. The reaction mixture was evaporated under a stream of N₂ and the residue and diluted with sat. aq. NaHCO₃ (3 ml), extracted with EtOAc (3×3 ml) and evaporated to dryness in vacuo. The residue was purified by column chromatography (SiO₂, 0-50% EtOAc/PE) to give the title compound as a light yellow solid (340 mg, 56%). LCMS m/z=318 [M+H]⁺.

### Intermediate B26. methyl 4-amino-1-(2,6-dimethyl-4-propoxyphenyl)-6-oxo-1,6-dihydropyrimidine-5-carboxylate

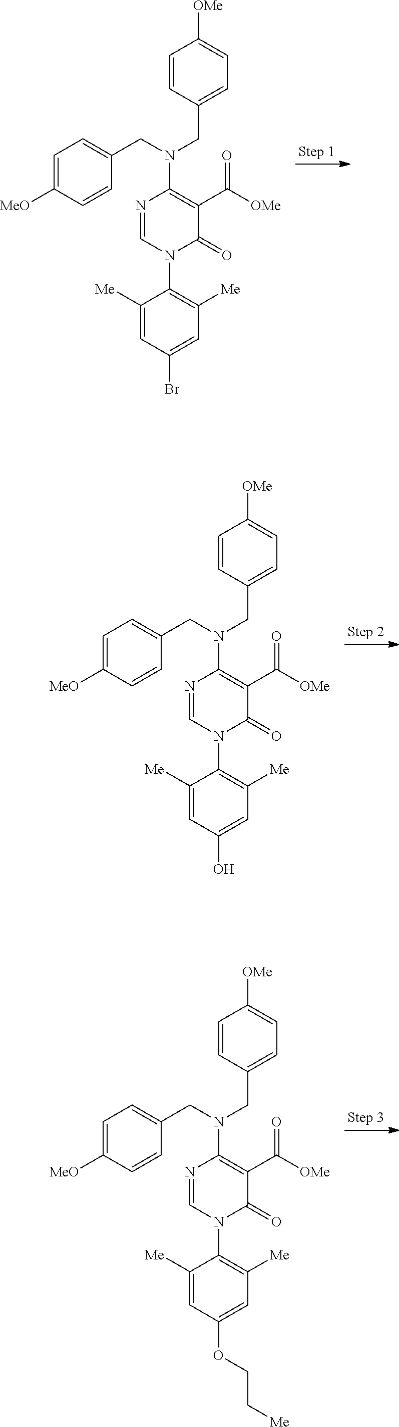 [SMILES: COC(=O)c1c(N(Cc2ccc(OC)cc2)Cc2ccc(OC)cc2)ncn(-c2c(C)cc(Br)cc2C)c1=O ; COC(=O)c1c(N(Cc2ccc(OC)cc2)Cc2ccc(OC)cc2)ncn(-c2c(C)cc(O)cc2C)c1=O ; CCCOc1cc(C)c(-n2cnc(N(Cc3ccc(OC)cc3)Cc3ccc(OC)cc3)c(C(=O)OC)c2=O)c(C)c1]

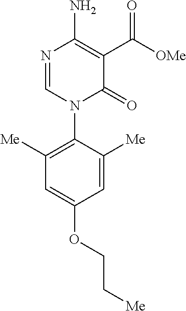 [SMILES: CCCOc1cc(C)c(-n2cnc(N)c(C(=O)OC)c2=O)c(C)c1]

The title compound was prepared as a white solid from methyl 4-(bis(4-methoxybenzyl)amino)-1-(4-bromo-2,6-dimethylphenyl)-6-oxo-1,6-dihydropyrimidine-5-carboxylate (Intermediate B18, Step 3) using an analogous 3-Step procedure as described for Intermediate B25 (Step 4, 5 and 6). LCMS m/z=332 [M+H]⁺.

### Intermediate B27. methyl 4-amino-1-(4-methoxy-2,6-dimethylphenyl)-6-oxo-1,6-dihydropyrimidine-5-carboxylate

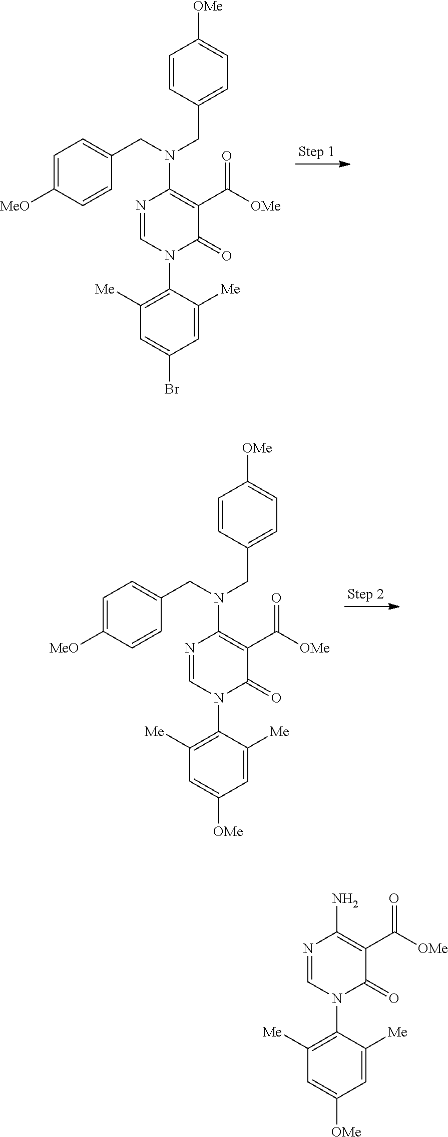 [SMILES: COC(=O)c1c(N(Cc2ccc(OC)cc2)Cc2ccc(OC)cc2)ncn(-c2c(C)cc(Br)cc2C)c1=O ; COC(=O)c1c(N(Cc2ccc(OC)cc2)Cc2ccc(OC)cc2)ncn(-c2c(C)cc(OC)cc2C)c1=O ; COC(=O)c1c(N)ncn(-c2c(C)cc(OC)cc2C)c1=O]

### Step 1. Synthesis of methyl 4-(bis(4-methoxybenzyl)amino)-1-(4-methoxy-2,6-dimethylphenyl)-6-oxo-1,6-dihydropyrimidine-5-carboxylate

To a solution of methyl 4-(bis(4-methoxybenzyl)amino)-1-(4-bromo-2,6-dimethylphenyl)-6-oxo-1,6-dihydropyrimidine-5-carboxylate (Intermediate B18, Step 3; 1.12 g, 1.89 mmol), Pd₂ (dba)₃ (173 mg, 0.189 mmol), t-Bu Xphos (80.3 mg, 0.189 mmol) and K₃PO₄ (602 mg, 2.84 mmol) in toluene (8 mL) and MeOH (8 mL) was stirred at 80° C. for 2 h under N₂. The reaction mixture was filtered and the filtrate concentrated under reduced pressure and the residue purified by column chromatography on silica gel (0-50% EtOAc/PE) to give the title compound as a brown oil (800 mg, 1.47 mmol, 77%). LCMS m/z=544 [M+H]⁺.

### Step 2. Synthesis of methyl 4-amino-1-(4-methoxy-2,6-dimethylphenyl)-6-oxo-1,6-dihydropyrimidine-5-carboxylate

To a solution of methyl 4-(bis(4-methoxybenzyl)amino)-1-(4-methoxy-2,6-dimethylphenyl)-6-oxo-1,6-dihydropyrimidine-5-carboxylate (Step 1, 1.20 g, 2.21 mmol) was dissolved in TFA (20 mL) and the mixture was stirred at 80° C. for 1 h. The reaction mixture was concentrated under reduced pressure and the pH of the residue adjusted to pH 6-7 with 1M Na₂CO₃. The residue was purified by column chromatography (0-50% EtOAc/PE) to give the title compound as a pale yellow solid (560 mg, 83%). LCMS m/z=304 [M+H]⁺.

### Intermediate B28. methyl 4-amino-1-(2,6-dichloro-4-(ethoxymethyl)phenyl)-6-oxo-1,6-dihydropyrimidine-5-carboxylate

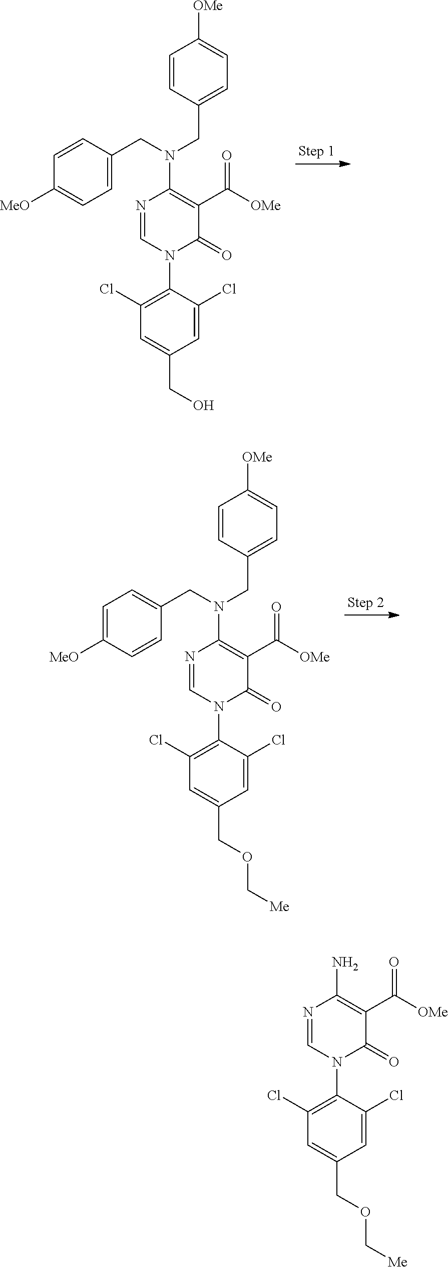 [SMILES: COC(=O)c1c(N(Cc2ccc(OC)cc2)Cc2ccc(OC)cc2)ncn(-c2c(Cl)cc(CO)cc2Cl)c1=O ; CCOCc1cc(Cl)c(-n2cnc(N(Cc3ccc(OC)cc3)Cc3ccc(OC)cc3)c(C(=O)OC)c2=O)c(Cl)c1 ; CCOCc1cc(Cl)c(-n2cnc(N)c(C(=O)OC)c2=O)c(Cl)c1]

### Step 1. Synthesis of methyl 4-(bis(4-methoxybenzyl)amino)-1-(2,6-dichloro-4-(ethoxymethyl)phenyl)-6-oxo-1,6-dihydropyrimidine-5-carboxylate

To a solution of methyl 4-(bis(4-methoxybenzyl)amino)-1-(2,6-dichloro-4-(hydroxymethyl)phenyl)-6-oxo-1,6-dihydropyrimidine-5-carboxylate (1.20 g, 2.05 mmol) in DCM (6 mL) was added DIPEA (796 mg, 6.16 mmol) to pH 10 followed by triethyloxonium tetrafluoroborate (1.56 g, 8.21 mmol) was added at 0° C. The mixture was stirred at 25° C. for 16 h, quenched by addition saturated aqueous Na₂CO₃ (10 mL) at 0° C., and extracted with DCM (3×10 mL). The combined organics were dried (Na₂SO₄) and evaporated to dryness in vacuo. The residue was purified by column chromatography (SiO₂, 0-25% EtOAc/PE) to give the title compound as a yellow solid (430 mg, 34%). LCMS m/z=588 [M+H]⁺.

### Step 2. Synthesis of methyl 4-amino-1-(2,6-dichloro-4-(ethoxymethyl)phenyl)-6-oxo-1,6-dihydropyrimidine-5-carboxylate

A mixture of methyl 4-(bis(4-methoxybenzyl)amino)-1-(2,6-dichloro-4-(ethoxymethyl)phenyl)-6-oxo-1,6-dihydropyrimidine-5-carboxylate (400 mg, 0.653 mmol) in TFA (5 mL) was stirred at 80° C. for 1 h. The reaction mixture was concentrated under reduced pressure and the pH of the residue adjusted to pH 8-9 with Na₂CO₃, diluted with H₂O (50 mL) and extracted with EtOAc (3×40 mL). The combined organics were washed with brine (2×30 mL), dried (Na₂SO₄) and concentrated under reduced pressure. The residue was purified by column chromatography (SiO₂, 0-50% EtOAc/PE) to give the title compound as a white solid (210 mg, 86%).

### Intermediate B29. methyl 4-amino-1-(4-(ethoxymethyl)-2,6-dimethylphenyl)-6-oxo-1,6-dihydropyrimidine-5-carboxylate

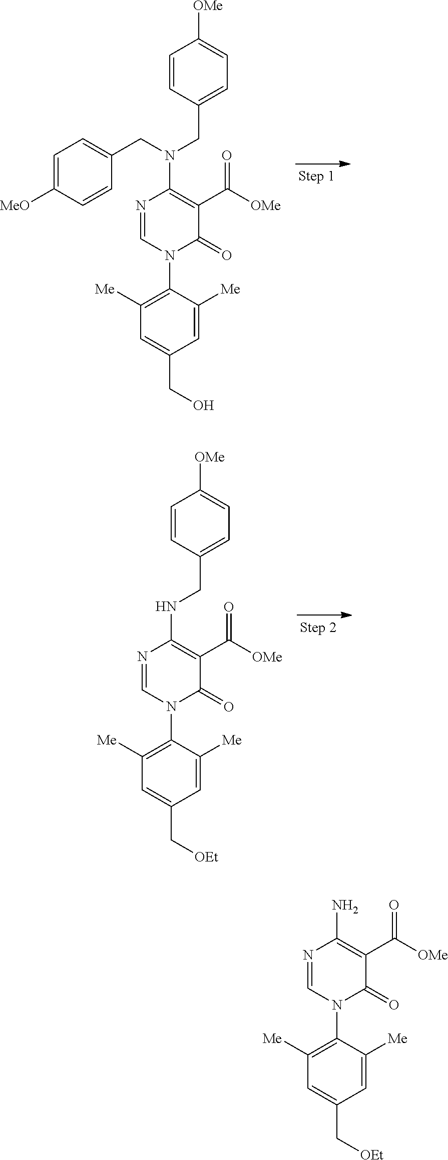 [SMILES: COC(=O)c1c(N(Cc2ccc(OC)cc2)Cc2ccc(OC)cc2)ncn(-c2c(C)cc(CO)cc2C)c1=O ; CCOCc1cc(C)c(-n2cnc(NCc3ccc(OC)cc3)c(C(=O)OC)c2=O)c(C)c1 ; CCOCc1cc(C)c(-n2cnc(N)c(C(=O)OC)c2=O)c(C)c1]

### Step 1. Synthesis of methyl 1-(4-(ethoxymethyl)-2,6-dimethylphenyl)-4-((4-methoxybenzyl)amino)-6-oxo-1,6-dihydropyrimidine-5-carboxylate

To a solution of methyl 4-(bis(4-methoxybenzyl)amino)-1-(4-(hydroxymethyl)-2,6-dimethylphenyl)-6-oxo-1,6-dihydropyrimidine-5-carboxylate (B22, Step 1, 1.00 g, 1.84 mmol) in DCM (5 mL) was added DIPEA (476 mg, 3.68 mmol) and triethyloxonium; tetrafluoroborate (1.40 g, 7.36 mmol) at 0° C. and the mixture stirred at 25° C. for 2 h. The reaction mixture was quenched by addition saturated aqueous NaHCO₃ (20 mL) at 0° C. and extracted with DCM (3×20 mL). The combined organics were dried (Na₂SO₄) and concentrated under reduced pressure to give a residue that was purified by prep-HPLC-(30-60% MeCN) to give the title compound as a white solid (170 mg, 20.5%). LCMS m/z=452 [M+H]⁺.

### Step 2. Synthesis of methyl 4-amino-1-(4-(ethoxymethyl)-2,6-dimethylphenyl)-6-oxo-1,6-dihydropyrimidine-5-carboxylate

Methyl 1-(4-(ethoxymethyl)-2,6-dimethylphenyl)-4-((4-methoxybenzyl)amino)-6-oxo-1,6-dihydropyrimidine-5-carboxylate (Step 1, 170 mg, 0.377 mol) was dissolved in TFA (2 mL) and the mixture was stirred at 80° C. for 1 h. The reaction mixture was quenched by addition saturated aqueous Na₂CO₃ solution (20 mL) and extracted with EtOAc (3×40 mL). The combined organics were dried (Na₂SO₄) and concentrated under reduced pressure. The residue was purified by MPLC (SiO₂, 50% EtOAc/PE) to give the title compound as a white solid (100 mg, 0.302 mmol, 80%). LCMS m/z=332 [M+H]⁺.

### Intermediate B30. methyl 4-amino-1-(2,6-dichloro-4-ethoxyphenyl)-6-oxo-1,6-dihydropyrimidine-5-carboxylate

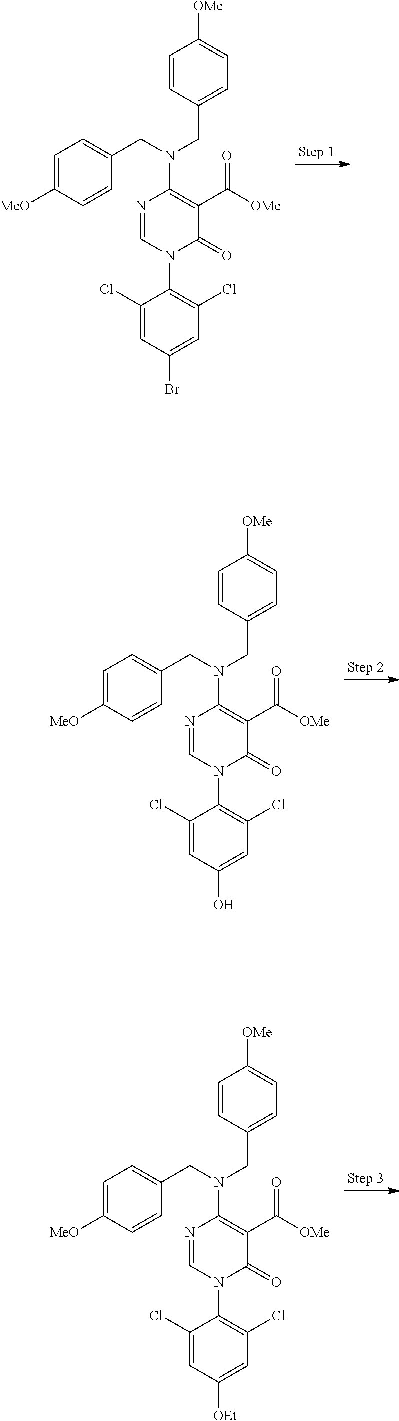 [SMILES: COC(=O)c1c(N(Cc2ccc(OC)cc2)Cc2ccc(OC)cc2)ncn(-c2c(Cl)cc(Br)cc2Cl)c1=O ; COC(=O)c1c(N(Cc2ccc(OC)cc2)Cc2ccc(OC)cc2)ncn(-c2c(Cl)cc(O)cc2Cl)c1=O ; CCOc1cc(Cl)c(-n2cnc(N(Cc3ccc(OC)cc3)Cc3ccc(OC)cc3)c(C(=O)OC)c2=O)c(Cl)c1]

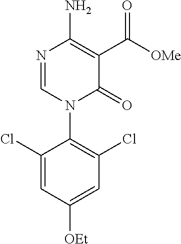 [SMILES: CCOc1cc(Cl)c(-n2cnc(N)c(C(=O)OC)c2=O)c(Cl)c1]

### Step 1. Synthesis of methyl 4-(bis(4-methoxybenzyl)amino)-1-(2,6-dichloro-4-hydroxyphenyl)-6-oxo-1,6-dihydropyrimidine-5-carboxylate

To a mixture of methyl 4-(bis(4-methoxybenzyl)amino)-1-(4-bromo-2,6-dichlorophenyl)-6-oxo-1,6-dihydropyrimidine-5-carboxylate (Intermediate B6 Step 3, 3.00 g, 4.74 mmol) in dioxane (30 mL) and H₂O (10 mL) was added K₃PO₄ (1.51 g, 7.11 mmol), t-Bu Xphos (201 mg, 0.474 mmol) and Pd₂ (dba)₃ (434 mg, 0.474 mmol) and the mixture stirred at 80° C. for 1 h under N₂. The reaction mixture was filtered and the filtrate concentrated under reduced pressure. The residue was purified by column chromatography (SiO₂, 0-50% EtOAc/PE) to give the title compound as a yellow solid (4 g). LCMS m/z=570 [M+H]⁺.

### Step 2. Synthesis of methyl 4-(bis(4-methoxybenzyl)amino)-1-(2,6-dichloro-4-ethoxyphenyl)-6-oxo-1,6-dihydropyrimidine-5-carboxylate

To a mixture of methyl 4-(bis(4-methoxybenzyl)amino)-1-(2,6-dichloro-4-hydroxyphenyl)-6-oxo-1,6-dihydropyrimidine-5-carboxylate (1.96 g, 3.44 mmol) and iodoethane (804 mg, 5.15 mmol) in DMF (15 mL) was added K₂CO₃ (950 mg, 6.87 mmol) and the mixture stirred at 60° C. for 2 h. The reaction mixture was quenched with H₂O (5.00 mL) at 0° C. and extracted with EtOAc (3×10 mL). The combined organics were dried (Na₂SO₄) and concentrated under reduced pressure. The residue was purified by column chromatography (SiO₂, 0-100% EtOAc/PE) to give the title compound as a yellow solid (1.70 g, 83%). LCMS m/z=598 [M+H]⁺.

### Step 3. Synthesis of methyl 4-amino-1-(2,6-dichloro-4-ethoxyphenyl)-6-oxo-1,6-dihydropyrimidine-5-carboxylate

A mixture of methyl 4-(bis(4-methoxybenzyl)amino)-1-(2,6-dichloro-4-ethoxyphenyl)-6-oxo-1,6-dihydropyrimidine-5-carboxylate (3.30 g, 5.51 mmol) in TFA (30 mL) was stirred at 80° C. for 1 hr. The reaction mixture was added to saturated aqueous NaHCO₃ solution (10 ml) and concentrated under reduced pressure and extracted with EtOAc (3×10 ml). The combined organics were dried (Na₂SO₄) and concentrated under reduced pressure. The residue was purified by column chromatography (SiO₂, 0-50% EtOAc/PE) to give the title compound as a white solid (1.10 g, 56% yield). LCMS m/z=358 [M+H]⁺.

### Intermediate B31. methyl 4-amino-1-(2,6-dichloro-4-isopropoxyphenyl)-6-oxo-1,6-dihydropyrimidine-5-carboxylate

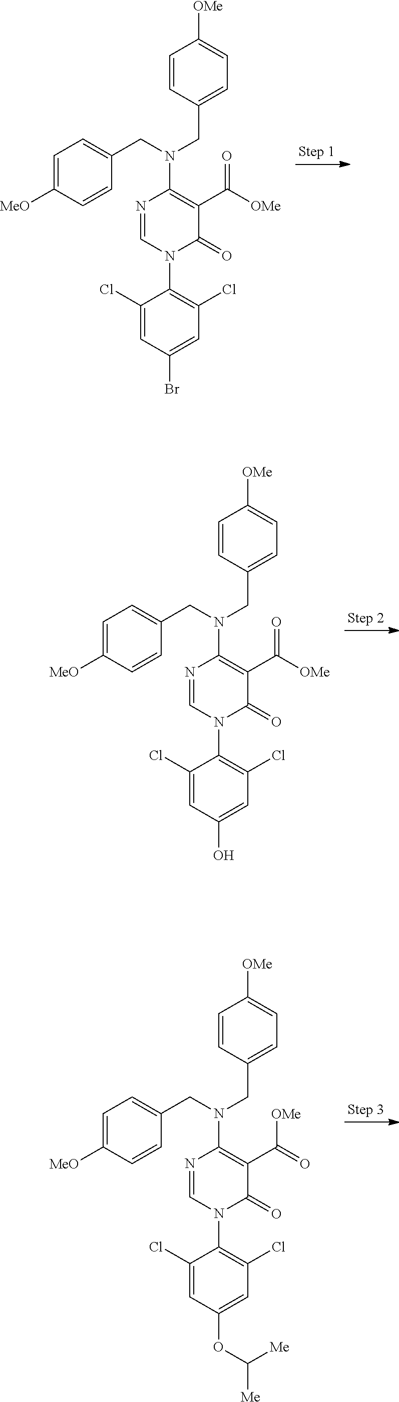 [SMILES: COC(=O)c1c(N(Cc2ccc(OC)cc2)Cc2ccc(OC)cc2)ncn(-c2c(Cl)cc(Br)cc2Cl)c1=O ; COC(=O)c1c(N(Cc2ccc(OC)cc2)Cc2ccc(OC)cc2)ncn(-c2c(Cl)cc(O)cc2Cl)c1=O ; COC(=O)c1c(N(Cc2ccc(OC)cc2)Cc2ccc(OC)cc2)ncn(-c2c(Cl)cc(OC(C)C)cc2Cl)c1=O]

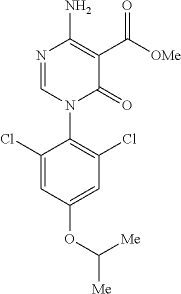 [SMILES: COC(=O)c1c(N)ncn(-c2c(Cl)cc(OC(C)C)cc2Cl)c1=O]

The title compound was prepared as a brown solid from methyl 4-(bis(4-methoxybenzyl)amino)-1-(4-bromo-2,6-dichlorophenyl)-6-oxo-1,6-dihydropyrimidine-5-carboxylate (Intermediate B6, Step 3) using an analogous 3-Step method as described for Intermediate B30. LCMS m/z=372 [M+H]⁺.

### Intermediate B32. methyl 4-amino-1-(4-(2,2-difluoroethyl)-2,6-dimethylphenyl)-6-oxo-1,6-dihydropyrimidine-5-carboxylate

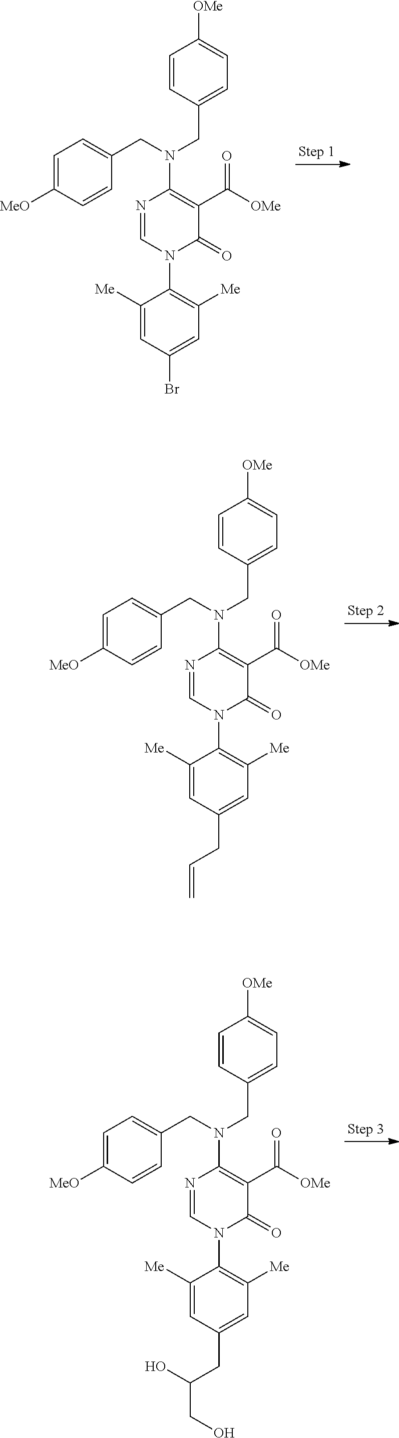 [SMILES: COC(=O)c1c(N(Cc2ccc(OC)cc2)Cc2ccc(OC)cc2)ncn(-c2c(C)cc(Br)cc2C)c1=O ; C=CCc1cc(C)c(-n2cnc(N(Cc3ccc(OC)cc3)Cc3ccc(OC)cc3)c(C(=O)OC)c2=O)c(C)c1 ; COC(=O)c1c(N(Cc2ccc(OC)cc2)Cc2ccc(OC)cc2)ncn(-c2c(C)cc(CC(O)CO)cc2C)c1=O]

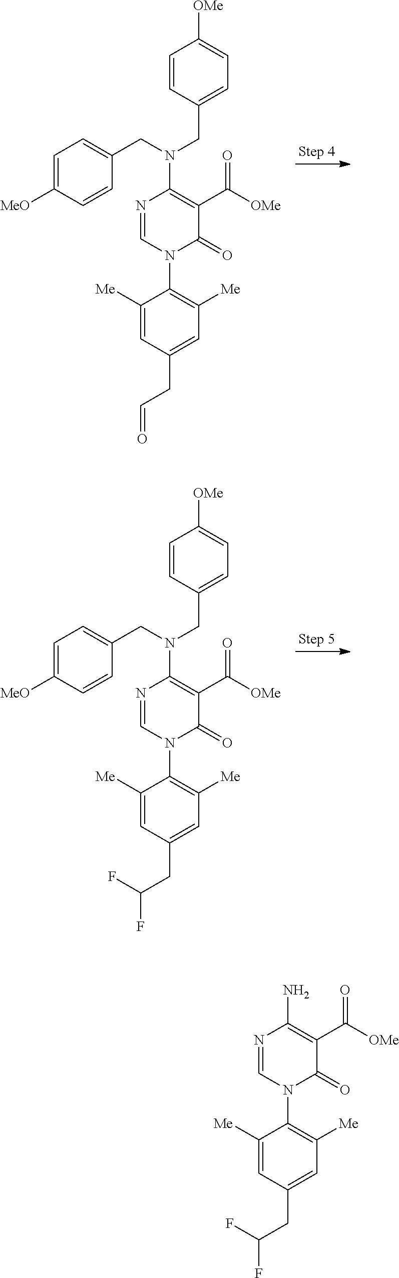 [SMILES: COC(=O)c1c(N(Cc2ccc(OC)cc2)Cc2ccc(OC)cc2)ncn(-c2c(C)cc(CC=O)cc2C)c1=O ; COC(=O)c1c(N(Cc2ccc(OC)cc2)Cc2ccc(OC)cc2)ncn(-c2c(C)cc(CC(F)F)cc2C)c1=O ; COC(=O)c1c(N)ncn(-c2c(C)cc(CC(F)F)cc2C)c1=O]

### Step 1. Synthesis of methyl 1-(4-allyl-2,6-dimethylphenyl)-4-(bis(4-methoxybenzyl)amino)-6-oxo-1,6-dihydropyrimidine-5-carboxylate

To a solution of methyl 4-(bis(4-methoxybenzyl)amino)-1-(4-bromo-2,6-dimethylphenyl)-6-oxo-1,6-dihydropyrimidine-5-carboxylate (Intermediate B18 Step 3, 500 mg, 0.844 mmol) in dioxane (6 mL) was added Pd(PPh₃)₄ (97.5 mg, 0.084 mol) and allyl(tributyl) stannane (1.08 g, 3.25 mmol) and the mixture stirred at 80° C. for 12 h under N₂. The reaction mixture was quenched by addition 4M KF (10 mL) at 0° C. and the mixture extracted with EtOAc (4×10 mL). The combined organics were concentrated under reduced pressure and the residue purified by MPLC (SiO₂, 1-50% EtOAc/PE) to give the title compound as a yellow solid (953 mg, crude). LCMS m/z=554 [M+H]⁺.

### Step 2. Synthesis of methyl 4-(bis(4-methoxybenzyl)amino)-1-(4-(2,3-dihydroxypropyl)-2,6-dimethylphenyl)-6-oxo-1,6-dihydropyrimidine-5-carboxylate

To a solution of methyl 1-(4-allyl-2,6-dimethylphenyl)-4-(bis(4-methoxybenzyl)amino)-6-oxo-1,6-dihydropyrimidine-5-carboxylate (903 mg, 1.63 mmol) in acetone (8 mL) and H₂O (1.20 mL) was added K₂OsO₄·2H₂O (60.1 mg, 0.163 mmol) and NMO (287 mg, 2.45 mmol) and the mixture stirred at 25° C. for 2 h under N₂. The reaction mixture was filtered and the filtrate evaporated to dryness in vacuo. The residue was purified by prep-HPLC (C18, 250×50 mm, 10 μm; 30-70% MeCN/H₂O(NH₄HCO₃)) to give the title compound as a grey solid (810 mg, 84.5%). LCMS m/z=588 [M+H]⁺.

### Step 3. Synthesis of methyl 4-(bis(4-methoxybenzyl)amino)-1-(2,6-dimethyl-4-(2-oxoethyl)phenyl)-6-oxo-1,6-dihydropyrimidine-5-carboxylate

To a solution of methyl 4-(bis(4-methoxybenzyl)amino)-1-(4-(2,3-dihydroxypropyl)-2,6-dimethylphenyl)-6-oxo-1,6-dihydropyrimidine-5-carboxylate (790 mg, 1.34 mmol) in MeOH (14 mL) was added NalO₄ (575 mg, 2.69 mmol) and the mixture stirred at 25° C. for 2 h. The reaction mixture was diluted with H₂O (20 mL) and extracted with EtOAc (3×10 mL). The combined organics were dried (Na₂SO₄) and evaporated to dryness in vacuo. The residue was purified by MPLC (SiO₂, 1-50% EtOAc/PE) to give the title compound as a white solid (810 mg, 84.5%). LCMS m/z=556 [M+H]⁺.

### Step 4. Synthesis of methyl 4-(bis(4-methoxybenzyl)amino)-1-(4-(2,2-difluoroethyl)-2,6-dimethylphenyl)-6-oxo-1,6-dihydropyrimidine-5-carboxylate

To a solution of methyl 4-(bis(4-methoxybenzyl)amino)-1-(2,6-dimethyl-4-(2-oxoethyl)phenyl)-6-oxo-1,6-dihydropyrimidine-5-carboxylate (Step 3, 580 mg, 1.04 mmol) in DCM (10 mL) was added DAST (1.68 g, 10.44 mmol) at −78° C. under N₂ and the mixture stirred at −78° C. for 0.5 h under N₂ and then at 25° C. for 1 h under N₂. The reaction mixture was quenched by addition saturated aqueous NaHCO₃ solution (20 mL) at 0° C. and extracted with EtOAc (3×10 mL). The combined organics were dried (Na₂SO₄) and evaporated to dryness and the residue purified by MPLC (SiO₂, 1-50% EtOAc/PE) to give the title compound as a white solid (450 mg, 74.6%). LCMS m/z=578 [M+H]⁺.

### Step 5. Synthesis of methyl 4-amino-1-(4-(2,2-difluoroethyl)-2,6-dimethylphenyl)-6-oxo-1,6-dihydropyrimidine-5-carboxylate

A mixture of methyl 4-(bis(4-methoxybenzyl)amino)-1-(4-(2,2-difluoroethyl)-2,6-dimethylphenyl)-6-oxo-1,6-dihydropyrimidine-5-carboxylate (440 mg, 0.762 mmol) in TFA (3 mL) was stirred at 80° C. for 0.5 h. The reaction mixture was concentrated under reduced pressure and the pH of the residue was adjusted with NaHCO₃ to pH 8-9. The mixture was diluted with H₂O (30 mL) and extracted with EtOAc (3×15 mL). The combined extracts were dried (Na₂SO₄) and concentrated under reduced pressure. The residue was purified by MPLC (SiO₂, 1-50% EtOAc/PE) to give the title compound as a white solid (190 mg, 74%). LCMS m/z=338 [M+H]⁺.

### Intermediate B33. methyl 4-amino-1-(4-(2-fluoroethyl)-2,6-dimethylphenyl)-6-oxo-1,6-dihydropyrimidine-5-carboxylate

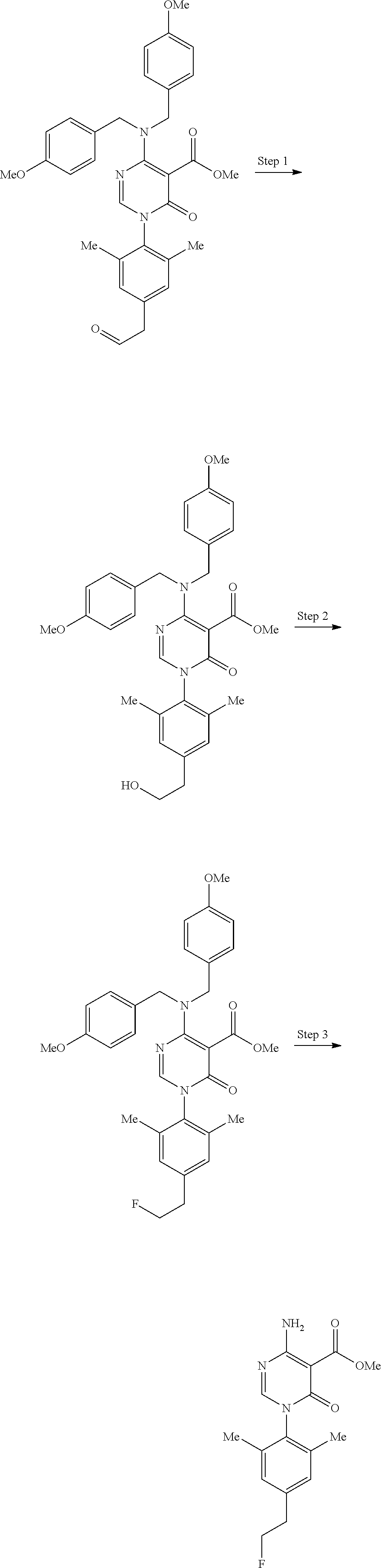 [SMILES: COC(=O)c1c(N(Cc2ccc(OC)cc2)Cc2ccc(OC)cc2)ncn(-c2c(C)cc(CC=O)cc2C)c1=O ; COC(=O)c1c(N(Cc2ccc(OC)cc2)Cc2ccc(OC)cc2)ncn(-c2c(C)cc(CCO)cc2C)c1=O ; COC(=O)c1c(N(Cc2ccc(OC)cc2)Cc2ccc(OC)cc2)ncn(-c2c(C)cc(CCF)cc2C)c1=O ; COC(=O)c1c(N)ncn(-c2c(C)cc(CCF)cc2C)c1=O]

### Step 1. Synthesis of methyl 4-(bis(4-methoxybenzyl)amino)-1-(4-(2-hydroxyethyl)-2,6-dimethylphenyl)-6-oxo-1,6-dihydropyrimidine-5-carboxylate

To a solution of methyl 4-(bis(4-methoxybenzyl)amino)-1-(2,6-dimethyl-4-(2-oxoethyl)phenyl)-6-oxo-1,6-dihydropyrimidine-5-carboxylate (Intermediate B32 Step 3, 650 mg, 1.17 mmol) in MeOH (5 mL) was added NaBH₄ (46.5 mg, 1.23 mmol) and the mixture was stirred at 25° C. for 10 min. The reaction mixture was quenched by addition aqueous HCl (1M, 2 mL) at 0° C. and extracted with EtOAc (3×20 mL). The combined organics were dried (Na₂SO₄), concentrated under reduced pressure and the residue purified by column chromatography on silica gel (0-100% EtOAc/PE) to give the title compound as a light yellow oil (440 mg, 67%). LCMS m/z=558 [M+H]⁺.

### Step 2. Synthesis of methyl 4-(bis(4-methoxybenzyl)amino)-1-(4-(2-fluoroethyl)-2,6-dimethylphenyl)-6-oxo-1,6-dihydropyrimidine-5-carboxylate

To a solution of methyl 4-(bis(4-methoxybenzyl)amino)-1-(4-(2-hydroxyethyl)-2,6-dimethylphenyl)-6-oxo-1,6-dihydropyrimidine-5-carboxylate (Step 1, 440 mg, 0.789 mmol) in DCM (4 mL) was added DAST (636 mg, 3.95 mmol) at −78° C. for 0.5 h and the mixture stirred at 25° C. for 1 h. The reaction mixture was quenched by addition saturated aqueous NaHCO₃ (5 mL) at 0° C. and extracted with EtOAc (3×10 mL). The combined organic layers were dried (Na₂SO₄), concentrated under reduced pressure and the residue purified by prep-TLC (EtOAc) to give the title compound as a pale yellow oil (310 mg, 70%). LCMS m/z=560 [M+H]⁺.

### Step 3. Synthesis of methyl 4-amino-1-(4-(2-fluoroethyl)-2,6-dimethylphenyl)-6-oxo-1,6-dihydropyrimidine-5-carboxylate

A solution of methyl 4-(bis(4-methoxybenzyl)amino)-1-(4-(2-fluoroethyl)-2,6-dimethylphenyl)-6-oxo-1,6-dihydropyrimidine-5-carboxylate (Step 2, 310 mg, 0.554 mmol) in TFA (1 mL) was stirred at 80° C. for 1 h. The reaction was quenched by addition saturated aqueous NaHCO₃ (5 mL) at 0° C. and then extracted with EtOAc (3×10 mL). The combined organics were dried (Na₂SO₄), concentrated under reduced pressure and the residue purified by prep-TLC (EtOAc) to give the title compound as a pale yellow oil (150 mg, 84%). LCMS m/z=320 [M+H]⁺.

### Intermediate B34. Synthesis of methyl 4-amino-1-(4-bromo-2,6-dimethylphenyl)-6-oxo-1,6-dihydropyrimidine-5-carboxylate

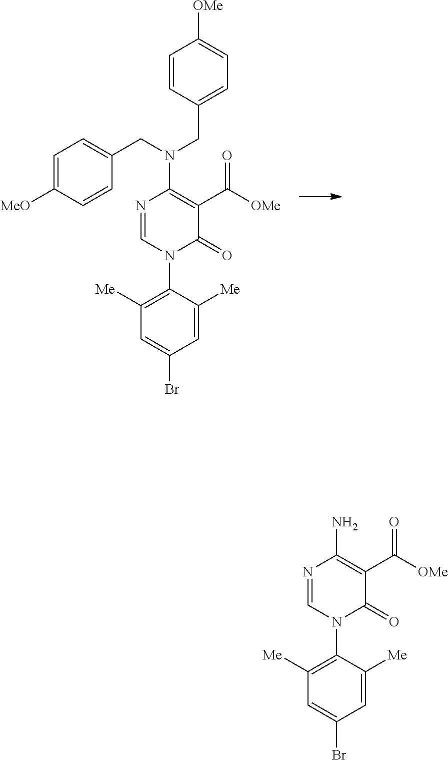 [SMILES: COC(=O)c1c(N(Cc2ccc(OC)cc2)Cc2ccc(OC)cc2)ncn(-c2c(C)cc(Br)cc2C)c1=O ; COC(=O)c1c(N)ncn(-c2c(C)cc(Br)cc2C)c1=O]

A solution of methyl 4-(bis(4-methoxybenzyl)amino)-1-(4-bromo-2,6-dimethylphenyl)-6-oxo-1,6-dihydropyrimidine-5-carboxylate (Intermediate B18 Step 3, 3.40 g, 5.74 mmol) in TFA (40 mL) was stirred at 80° C. for 0.5 h. The mixture was concentrated under reduced pressure and the residue partitioned between DCM (40 mL) and Na₂CO₃ (15 mL). The water phase was extracted with DCM (3×40 mL) and the combined organics dried (Na₂SO₄) and evaporated to dryness in vacuo. The residue was purified by MPLC (SiO₂, 0-5-% EtOAc/PE) to give the title compound as a yellow solid (2 g, crude). ¹H NMR (400 MHz, CDCl₃): 8.90 (s, 1H), 7.59 (s, 1H), 7.25 (s, 2H), 3.80 (s, 3H), 2.05 (s, 6H).

### Intermediate B35. Synthesis of methyl 4-amino-1-mesityl-6-oxo-1,6-dihydropyrimidine-5-carboxylate

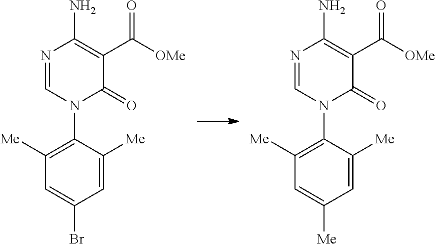 [SMILES: COC(=O)c1c(N)ncn(-c2c(C)cc(Br)cc2C)c1=O ; COC(=O)c1c(N)ncn(-c2c(C)cc(C)cc2C)c1=O]

To the mixture of methyl 4-amino-1-(4-bromo-2,6-dimethylphenyl)-6-oxo-1,6-dihydropyrimidine-5-carboxylate (Intermediate B34, 300 mg, 0.852 mmol) and 2,4,6-trimethyl-1,3,5,2,4,6-trioxatriborinane (1.07 g, 4.26 mmol) in dioxane (5 mL) was added Xphos-Pd-G2 (67.02 mg, 0.085 μmol) and K₃PO₄ (271 mg, 1.28 mmol) and the mixture stirred at 80° C. for 12 h under N₂. The reaction mixture was filtered and the filtrate was concentrated under reduced pressure and the residue purified by column chromatography (SiO₂; 1-50% EtOAc/PE) to give the title compound as a white solid (350 mg, 71.5%). LCMS m/z=288 [M+H]⁺.

### Intermediate B36. Synthesis of tert-butyl ((5-(4-amino-1-(4-(difluoromethyl)-2,6-dimethylphenyl)-6-oxo-1,6-dihydropyrimidine-5-carboxamido)pyridin-3-yl)methyl)(methyl)carbamate

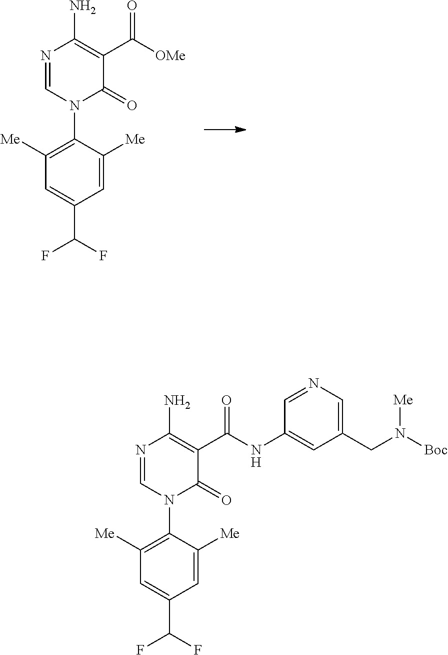 [SMILES: COC(=O)c1c(N)ncn(-c2c(C)cc(C(F)F)cc2C)c1=O ; Cc1cc(C(F)F)cc(C)c1-n1cnc(N)c(C(=O)Nc2cncc(CN(C)C(=O)OC(C)(C)C)c2)c1=O]

To a mixture of methyl 4-amino-1-(4-(difluoromethyl)-2,6-dimethylphenyl)-6-oxo-1,6-dihydropyrimidine-5-carboxylate (Intermediate B17, 50 mg, 0.155 mmol) and tert-butyl ((5-aminopyridin-3-yl)methyl)(methyl)carbamate (55.1 mg, 0.232 mmol) in toluene (4 mL) was added AlMe₃ (2 M, 0.232 mL) at 0° C. and the mixture stirred at 0° C. for another 5 min. The mixture was stirred at 100° C. for 1 h under N₂. The reaction mixture was quenched with H₂O (1 mL) and TFA (1 mL) at 0° C. and concentrated under reduced pressure to give the title compound as a white solid (80 mg, 98%). LCMS m/z=529 [M+H]⁺.

### Intermediate B37. methyl 4-amino-1-(2,6-dichloro-4-(trifluoromethoxy)phenyl)-6-oxo-1,6-dihydropyrimidine-5-carboxylate

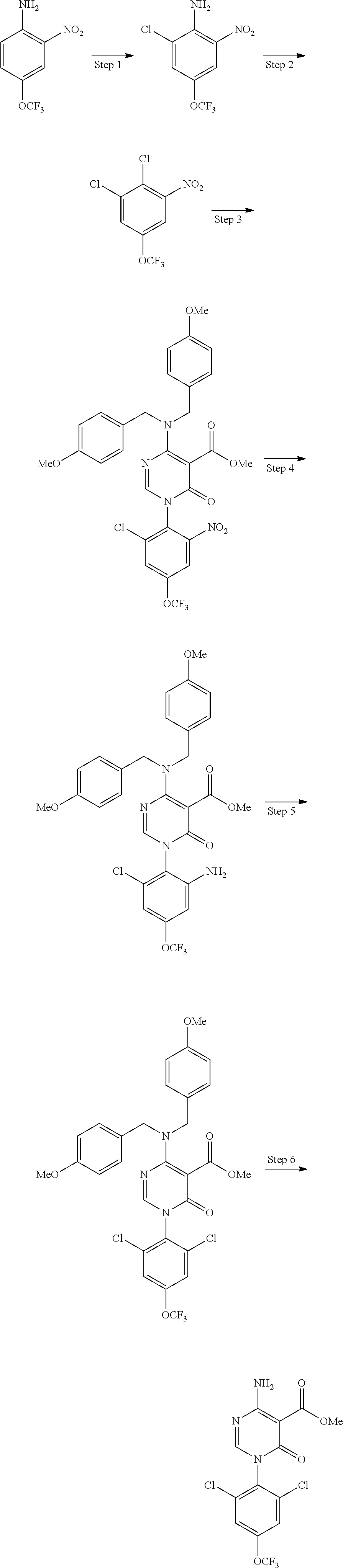

### Step 1. Synthesis of 2-chloro-6-nitro-4-(trifluoromethoxy) aniline

To a solution of 2-nitro-4-(trifluoromethoxy) aniline (8.4 g, 37.82 mmol) in AcOH (15 mL) was added sulfuryl chloride (7.66 g, 56.73 mmol, 5.67 mL) at 0° C. The mixture was stirred at 25° C. for 12 h and then evaporated to dryness in vacuo. The residue was purified by column chromatography (SiO₂, 25-100% EtOAc/PE) to give the title compound as a yellow solid (7.4 g, 76%). ¹H NMR (400 MHz, DMSO-d₆): 8.05-8.01 (m, 1H), 7.97 (d, 1H), 7.51 (br s, 2H).

### Step 2. Synthesis of 1,2-dichloro-3-nitro-5-(trifluoromethoxy)benzene To a mixture of 2-chloro-6-nitro-4-(trifluoromethoxy) aniline (5 g, 19.49 mmol) in MeCN (30 mL) was added CuCl₂ (3.14 g, 23.4 mmol) at 25° C. followed by tert-butyl nitrite (3.01 g, 29.23 mmol) and the mixture stirred at 60° C. for 2 h. The reaction mixture was concentrated under reduced pressure and the residue purified by prep-TLC (1:10 EtOAc/PE) to give the title compound as a white solid (4.1 g, 76%). ¹H NMR (400 MHz, DMSO-d₆): 8.36-8.32 (m, 1H), 8.30-8.27 (m, 1H)

### Step 3. Synthesis of methyl 4-(bis(4-methoxybenzyl)amino)-1-(2-chloro-6-nitro-4-(trifluoromethoxy)phenyl)-6-oxo-1,6-dihydropyrimidine-5-carboxylate

To a solution of methyl 4-(bis(4-methoxybenzyl)amino)-6-oxo-1,6-dihydropyrimidine-5-carboxylate (Intermediate B1, 5 g, 12.21 mmol) and 1,2-dichloro-3-nitro-5-(trifluoromethoxy)benzene (Step 2, 3.71 g, 13.43 mmol) in DMF (15 mL) was added K₂CO₃ (3.38 g, 24.42 mmol) and the mixture stirred at 80° C. for 12 h. The reaction mixture was filtered and the filtrate concentrated under reduced pressure and the residue was purified by prep-HPLC-26 (50-80% MeCN) to give the title compound as a white solid (1.9 g, 24%). LCMS m/z=649 [M+H]⁺.

### Step 4. Synthesis of methyl 1-(2-amino-6-chloro-4-(trifluoromethoxy)phenyl)-4-(bis(4-methoxybenzyl)amino)-6-oxo-1,6-dihydropyrimidine-5-carboxylate

To a solution of methyl 4-(bis(4-methoxybenzyl)amino)-1-(2-chloro-6-nitro-4-(trifluoromethoxy)phenyl)-6-oxo-1,6-dihydropyrimidine-5-carboxylate (Step 3, 1.6 g, 2.47 mmol) in H₂O (10 mL) and EtOH (30 mL) was added Fe (1.38 g, 24.7 mmol) and NH₄Cl (1.32 g, 24.7 mmol) and the mixture stirred at 80° C. for 6 h. The reaction mixture was filtered and the filtrate was concentrated under reduced pressure and the residue purified by column chromatography (SiO₂, 0-50% EtOAc/PE) to give the title compound as a yellow solid (850 mg, 55%). LCMS m/z=619 [M+H]⁺.

### Step 5. Synthesis of methyl 4-(bis(4-methoxybenzyl)amino)-1-(2,6-dichloro-4-(trifluoromethoxy)phenyl)-6-oxo-1,6-dihydropyrimidine-5-carboxylate

Isopentyl nitrite (454 mg, 3.88 mmol) in MeCN (3 mL) was added dropwise to a mixture of methyl 1-(2-amino-6-chloro-4-(trifluoromethoxy)phenyl)-4-(bis(4-methoxybenzyl)amino)-6-oxo-1,6-dihydropyrimidine-5-carboxylate (800 mg, 1.29 mmol), CuCl (256 mg, 2.58 mmol) and CuCl₂ (521 mg, 3.88 mmol) in MeCN (10 mL) and the mixture stirred at 25° C. for 1 h under N₂. The residue was diluted with H₂O (20 mL) and extracted with EtOAc (3×20 mL). The combined organics were dried (Na₂SO₄) and concentrated under reduced pressure to give the title compound as a white solid (800 mg, 97% yield). LCMS m/z=639 [M+H]⁺.

### Step 6. Synthesis of methyl 4-amino-1-(2,6-dichloro-4-(trifluoromethoxy)phenyl)-6-oxo-1,6-dihydropyrimidine-5-carboxylate

A mixture of methyl 4-(bis(4-methoxybenzyl)amino)-1-(2,6-dichloro-4-(trifluoromethoxy)phenyl)-6-oxo-1,6-dihydropyrimidine-5-carboxylate (600 mg, 0.94 mmol) in TFA (3 mL) was stirred at 80° C. for 1 h. The reaction mixture was concentrated under reduced pressure and the pH of the residue adjusted to pH 6-7 with 1M Na₂CO₃ (10 ml) and the residue purified by column chromatography (SiO₂, 0-50% EtOAc/PE) to give the title compound as a yellow solid (330 mg, 88%). LCMS m/z=398 [M+H]⁺.

### Intermediate B38. Synthesis of methyl 4-amino-1-(2,6-dimethyl-4-(trifluoromethoxy)phenyl)-6-oxo-1,6-dihydropyrimidine-5-carboxylate

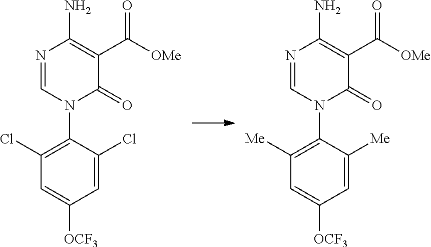 [SMILES: COC(=O)c1c(N)ncn(-c2c(Cl)cc(OC(F)(F)F)cc2Cl)c1=O ; COC(=O)c1c(N)ncn(-c2c(C)cc(OC(F)(F)F)cc2C)c1=O]

A mixture of methyl 4-amino-1-(2,6-dichloro-4-(trifluoromethoxy)phenyl)-6-oxo-1,6-dihydropyrimidine-5-carboxylate (Intermediate B37, 180 mg, 0.452 mmol), 2,4,6-trimethyl-1,3,5,2,4,6-trioxatriborinane (568 mg, 2.26 mmol), XPHOS-PD-G2 (35.6 mg, 0.045 mmol), K₃PO₄ (192 mg, 0.904 mmol) in dioxane (6 mL) was degassed and purged with N₂ (3×) and the mixture stirred at 80° C. for 6 h under N₂. The reaction mixture was filtered and the filtrate was concentrated under reduced pressure and the residue purified by prep-TLC (50% EtOAc/PE) to give the title compound as a yellow solid (130 mg, 80%). LCMS m/z=358 [M+H]⁺.

### Intermediate B39. Synthesis of methyl 4-amino-1-(2,6-dichloro-4-fluorophenyl)-6-oxo-1,6-dihydropyrimidine-5-carboxylate

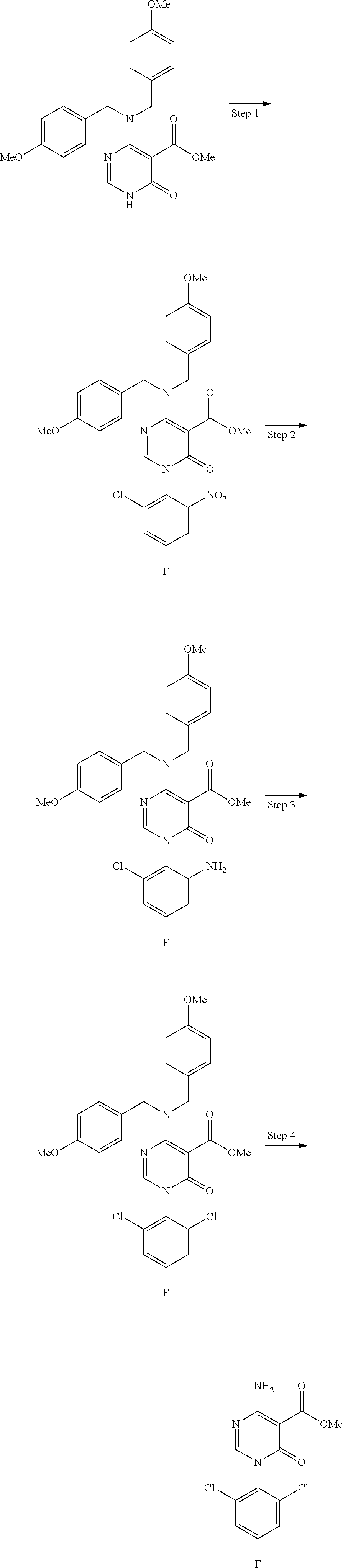 [SMILES: COC(=O)c1c(N(Cc2ccc(OC)cc2)Cc2ccc(OC)cc2)nc[nH]c1=O ; COC(=O)c1c(N(Cc2ccc(OC)cc2)Cc2ccc(OC)cc2)ncn(-c2c(Cl)cc(F)cc2[N+](=O)[O-])c1=O ; COC(=O)c1c(N(Cc2ccc(OC)cc2)Cc2ccc(OC)cc2)ncn(-c2c(N)cc(F)cc2Cl)c1=O ; COC(=O)c1c(N(Cc2ccc(OC)cc2)Cc2ccc(OC)cc2)ncn(-c2c(Cl)cc(F)cc2Cl)c1=O ; COC(=O)c1c(N)ncn(-c2c(Cl)cc(F)cc2Cl)c1=O]

The title compound was prepared from methyl 4-(bis(4-methoxybenzyl)amino)-6-oxo-1,6-dihydropyrimidine-5-carboxylate (Intermediate B1) and 1,2-dichloro-5-fluoro-3-nitrobenzene using an analogous method to that described in Steps 3-6 for Intermediate B37. LCMS m/z=332 [M+H]⁺.

### Intermediate B40 and B41. methyl(S)-4-amino-1-(4-(1,2-difluoroethyl)-2,6-dimethylphenyl)-6-oxo-1,6-dihydropyrimidine-5-carboxylate and methyl(R)-4-amino-1-(4-(1,2-difluoroethyl)-2,6-dimethylphenyl)-6-oxo-1,6-dihydropyrimidine-5-carboxylate

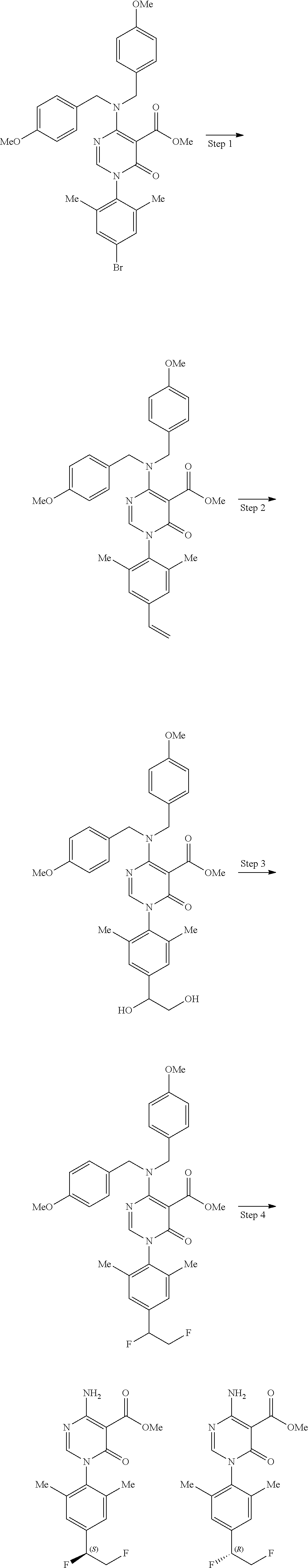

### Step 1. Synthesis of methyl 4-(bis(4-methoxybenzyl)amino)-1-(2,6-dimethyl-4-vinylphenyl)-6-oxo-1,6-dihydropyrimidine-5-carboxylate

A mixture of methyl 4-(bis(4-methoxybenzyl)amino)-1-(4-bromo-2,6-dimethylphenyl)-6-oxo-1,6-dihydropyrimidine-5-carboxylate (Intermediate B18 Step 3, 3 g, 5.06 mmol), 4,4,5,5-tetramethyl-2-vinyl-1,3,2-dioxaborolane (1.17 g, 7.60 mmol), K₂CO₃ (1.40 g, 10.13 mmol), Pd(dppf) C₁₂ (370.5 mg, 0.51 mmol) in dioxane (30 mL) and H₂O (2 mL) was degassed and purged with N₂ (×3) and the mixture stirred at 100° C. for 1 h under N₂. The solids were removed by filtration and the filtrate evaporated to dryness in vacuo. The residue was purified by column chromatography (SiO₂, 0-50 EtOAc/PE) to give the title compound as a yellow solid (2.6 g, 95%). LCMS m/z=540 [M+H]⁺.

### Step 2. Synthesis of methyl 4-(bis(4-methoxybenzyl)amino)-1-(4-(1,2-dihydroxyethyl)-2,6-dimethylphenyl)-6-oxo-1,6-dihydropyrimidine-5-carboxylate

A mixture of methyl 4-(bis(4-methoxybenzyl)amino)-1-(2,6-dimethyl-4-vinylphenyl)-6-oxo-1,6-dihydropyrimidine-5-carboxylate (2.57 g, 4.76 mmol), 4-methylmorpholine 4-oxide (837 mg, 7.14 mmol) and K₂OsO₄·2H₂O (175 mg, 0.476 mmol) in acetone (30 mL) and H₂O (4 mL) was degassed and purged with N₂ (×3) and the mixture stirred at 25° C. for 2 h under N₂. The reaction mixture was partitioned between DCM (30 mL) and H₂O (30 mL) and the aqueous phase extracted with DCM (3×30 mL). The combined organics were dried (Na₂SO₄) and evaporated to dryness in vacuo. The residue was purified by column chromatography (SiO₂, 0-100% EtOAc/PE) to give the title compound as a yellow solid (1.15 g, 42%). LCMS m/z=574 [M+H]⁺.

### Step 3. Synthesis of methyl 4-(bis(4-methoxybenzyl)amino)-1-(4-(1,2-difluoroethyl)-2,6-dimethylphenyl)-6-oxo-1,6-dihydropyrimidine-5-carboxylate

DAST (843 mg, 5.23 mmol) was added to a solution of methyl 4-(bis(4-methoxybenzyl)amino)-1-(4-(1,2-dihydroxyethyl)-2,6-dimethylphenyl)-6-oxo-1,6-dihydropyrimidine-5-carboxylate (300 mg, 0.523 mmol) in DCM (20 mL) and the mixture stirred at −78° C. for 0.5 h and then stirred at 25° C. for 1 h under N₂. The reaction mixture was quenched by addition of saturated aqueous NaHCO₃ (20 mL) at 0° C. and extracted with DCM (3×20 mL). The combined organics were dried (Na₂SO₄) and evaporated to dryness in vacuo to afford the title compound as a yellow solid (800 mg, 88%). LCMS m/z=578 [M+H]⁺.

### Step 4. Synthesis of methyl(S)-4-amino-1-(4-(1,2-difluoroethyl)-2,6-dimethylphenyl)-6-oxo-1,6-dihydropyrimidine-5-carboxylate and methyl(R)-4-amino-1-(4-(1,2-difluoroethyl)-2,6-dimethylphenyl)-6-oxo-1,6-dihydropyrimidine-5-carboxylate

A solution of methyl 4-(bis(4-methoxybenzyl)amino)-1-(4-(1,2-difluoroethyl)-2,6-dimethylphenyl)-6-oxo-1,6-dihydropyrimidine-5-carboxylate (700 mg, 1.21 mmol) in TFA (20 mL) was stirred at 80° C. for 1 h under N₂. The reaction mixture was evaporated to dryness in vacuo and the residue dissolved in THF (20 ml) and the pH was adjusted with NH₄OH to pH 9-11 at 0° C. The mixture was concentrated under reduced pressure and the residue was purified by column chromatography (SiO₂, 0-50% EtOAc/PE) to afford methyl 4-amino-1-(4-(1,2-difluoroethyl)-2,6-dimethylphenyl)-6-oxo-1,6-dihydropyrimidine-5-carboxylate which was separated by chiral SFC (DAICEL CHIRALCEL OJ (250×30 mm, 10 μm); 13% (0.1% NH₄OH/IPA) in CO₂) to afford:

Intermediate B40, Peak 1; methyl(S)-4-amino-1-(4-(1,2-difluoroethyl)-2,6-dimethylphenyl)-6-oxo-1,6-dihydropyrimidine-5-carboxylate or methyl(R)-4-amino-1-(4-(1,2-difluoroethyl)-2,6-dimethylphenyl)-6-oxo-1,6-dihydropyrimidine-5-carboxylate (yellow solid, 100 mg). LCMS m/z=338 [M+H]⁺.

Intermediate B41, Peak 2; methyl(S)-4-amino-1-(4-(1,2-difluoroethyl)-2,6-dimethylphenyl)-6-oxo-1,6-dihydropyrimidine-5-carboxylate or methyl(R)-4-amino-1-(4-(1,2-difluoroethyl)-2,6-dimethylphenyl)-6-oxo-1,6-dihydropyrimidine-5-carboxylate (yellow solid, 110 mg). LCMS m/z=338 [M+H]⁺.

### Intermediate B42 and B43. methyl(S)-4-amino-1-(2,6-dichloro-4-(1,2-difluoroethyl)phenyl)-6-oxo-1,6-dihydropyrimidine-5-carboxylate and methyl(R)-4-amino-1-(2,6-dichloro-4-(1,2-difluoroethyl)phenyl)-6-oxo-1,6-dihydropyrimidine-5-carboxylate

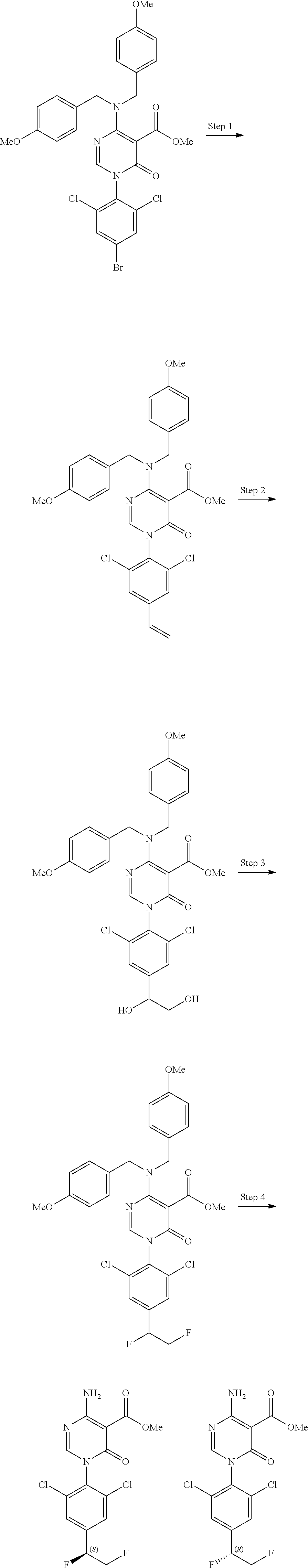

The title compounds were prepared from methyl 4-(bis(4-methoxybenzyl)amino)-1-(4-bromo-2,6-dichlorophenyl)-6-oxo-1,6-dihydropyrimidine-5-carboxylate (Intermediate B6 Step 3) using an analogous 4 step procedure as described for Intermediates B40 and B41. Chiral SFC (DAICEL CHIRALCEL OJ (250×30 mm, 10 μm); 30% (0.1% NH₄OH/IPA) in CO₂) to afford:

### Intermediate B42, Peak 1; methyl(S)-4-amino-1-(2,6-dichloro-4-(1,2-difluoroethyl)phenyl)-6-oxo-1,6-dihydropyrimidine-5-carboxylate or methyl(R)-4-amino-1-(2,6-dichloro-4-(1,2-difluoroethyl)phenyl)-6-oxo-1,6-dihydropyrimidine-5-carboxylate (white solid, 250 mg, 25.6%). LCMS m/z=378 [M+H]⁺

### Intermediate B43, Peak 2; methyl(S)-4-amino-1-(2,6-dichloro-4-(1,2-difluoroethyl)phenyl)-6-oxo-1,6-dihydropyrimidine-5-carboxylate or methyl(R)-4-amino-1-(2,6-dichloro-4-(1,2-difluoroethyl)phenyl)-6-oxo-1,6-dihydropyrimidine-5-carboxylate (white solid, 300 mg, 30.7%). LCMS m/z=378 [M+H]⁺

### Intermediate B44. methyl 4-amino-1-(2-chloro-4-fluoro-6-methylphenyl)-6-oxo-1,6-dihydropyrimidine-5-carboxylate

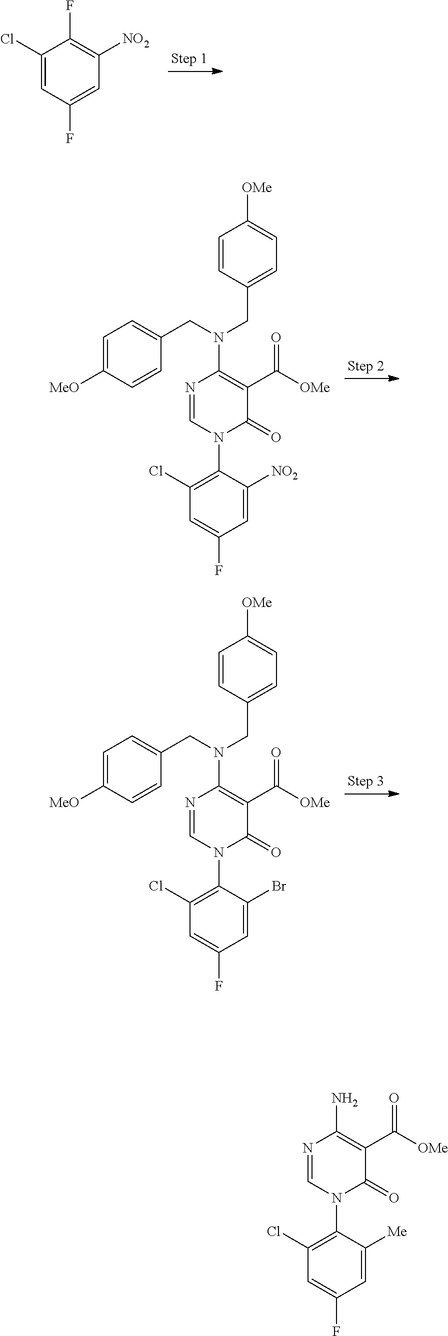

### Part 1. Synthesis of methyl 4-(bis(4-methoxybenzyl)amino)-1-(2-chloro-4-fluoro-6-nitrophenyl)-6-oxo-1,6-dihydropyrimidine-5-carboxylate

To a solution of 1-chloro-2,5-difluoro-3-nitrobenzene (539 mg, 2.75 mmol) in anhydrous MeCN (8 mL) were added methyl 4-(bis(4-methoxybenzyl)amino)-6-oxo-1,6-dihydropyrimidine-5-carboxylate (Intermediate B1, 1.03 g, 2.50 mmol) and K₂CO₃ (701 mg, 5.00 mmol) and the mixture heated to 80° C. for 2 h. The reaction was cooled, poured into H₂O (100 mL) and extracted with EtOAc (4×30 mL). The combined organics were washed twice with water, brine, dried (MgSO₄) and concentrated by rotary evaporation. The residue was purified by flash chromatography (ISCO 40 g silica, 0-10% MeOH/DCM) to afford the title compound as a yellow foam (1.29 g, 89%). LCMS m/z=583 [M+H]⁺.

### Step 2. Synthesis of methyl 4-(bis(4-methoxybenzyl)amino)-1-(2-bromo-6-chloro-4-fluorophenyl)-6-oxo-1,6-dihydropyrimidine-5-carboxylate

A suspension of methyl 4-(bis(4-methoxybenzyl)amino)-1-(2-chloro-4-fluoro-6-nitrophenyl)-6-oxo-1,6-dihydropyrimidine-5-carboxylate (Step 1, 1.25 g, 2.15 mmol) and iron powder (811 mg, 12.90 mmol) in MeOH (22 mL)/glacial acetic acid (2 mL) was heated to 50° C. for 4 h. The reaction was cooled and residual iron was removed with a magnet. The mixture was poured into H₂O (200 mL) and stirred at rt for 15 min to generate a precipitate. The precipitate was collected by filtration, washed with H₂O (200 mL) and dried to provide a beige powder which was dissolved in MeCN (25 mL). To the was added 48% aqueous HBr (0.61 mL, 5.38 mmol) and the mixture cooled on ice before tert-Butyl nitrite (0.31 mL, 5.16 mmol) was added and the mixture was stirred at 0° C. for 45 min and then CuBr (383 mg, 2.58 mmol) added at 0° C. and the reaction mixture left in the ice bath to gradually warm to rt. Another portion of tBuONO (0.30 mL, 2.58 mmol) was added and stirring continued for 1.5 h. The mixture was filtered washing with EtOAc and MeOH. The combined organics were evaporated to dryness and the residue dissolved in EtOAc (75 mL) and dilute aqueous HCl (150 mL). The aqueous component was extracted with EtOAc (2×50 mL). The combined organics were washed with saturated aqueous NaHCO₃, water, brine, dried (MgSO₄) and evaporated to dryness in vacuo. The residue was purified by flash chromatography (ISCO 40 g silica, 0-100% EtOAc/hex) to give the title compound as a light yellow foam (748 mg, 56% yield over 2 steps). LCMS m/z=616 [M+H]⁺.

### Step 3. Synthesis of methyl 4-(bis(4-methoxybenzyl)amino)-1-(2-chloro-4-fluoro-6-methylphenyl)-6-oxo-1,6-dihydropyrimidine-5-carboxylate

To a suspension of methyl 4-(bis(4-methoxybenzyl)amino)-1-(2-bromo-6-chloro-4-fluorophenyl)-6-oxo-1,6-dihydropyrimidine-5-carboxylate (Step 2, 505 mg, 0.81 mmol), K₂CO₃ (224 mg, 1.62 mmol), and Pd(dppf)Cl₂ DCM adduct (40.2 mg, 0.041 mmol) in dioxane (3 mL) was added trimethylboroxine solution (50 wt % in THF, 0.30 mL, 1.05 mmol). The mixture was purged with N₂ for 10 min and heated to 100° C. for 90 min. The reaction was cooled, filtered through Celite and washed with EtOAc. The combined organics were evaporated to dryness and the residue purified by flash chromatography (ISCO 40g silica, 0-70% EtOAc/hex) afforded an off-white foam. The foam was dissolved in TFA (3.5 mL) and heated to 80° C. for 1 h. The reaction was then cooled, poured into saturated aqueous NaHCO₃ and extracted with EtOAc (3×25 mL). The combined organics were washed with water, brine, dried (MgSO₄) and concentrated under reduced pressure. The residue was purified by flash chromatography (ISCO 12 g silica, 10-80% EtOAc/hex) to afford the title compound as an off-white solid (155 mg, 61% yield over 2 steps). LCMS m/z=312 [M+H]⁺.

### Intermediate B45. methyl 4-amino-1-(2,6-dichloro-4-(difluoromethoxy)phenyl)-6-oxo-1,6-dihydropyrimidine-5-carboxylate

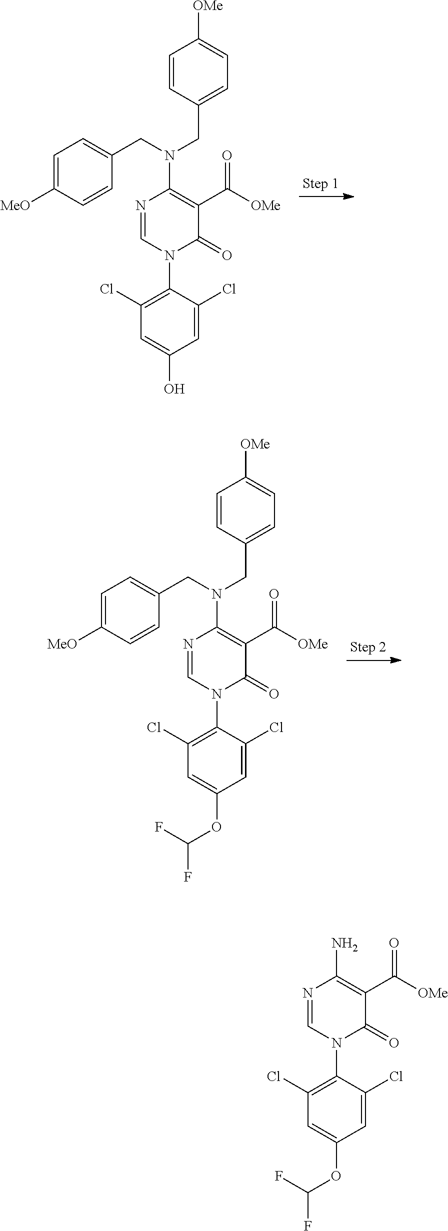 [SMILES: COC(=O)c1c(N(Cc2ccc(OC)cc2)Cc2ccc(OC)cc2)ncn(-c2c(Cl)cc(O)cc2Cl)c1=O ; COC(=O)c1c(N(Cc2ccc(OC)cc2)Cc2ccc(OC)cc2)ncn(-c2c(Cl)cc(OC(F)F)cc2Cl)c1=O ; COC(=O)c1c(N)ncn(-c2c(Cl)cc(OC(F)F)cc2Cl)c1=O]

### Step 1. Synthesis of methyl 4-(bis(4-methoxybenzyl)amino)-1-(2,6-dichloro-4-(difluoromethoxy)phenyl)-6-oxo-1,6-dihydropyrimidine-5-carboxylate

To a solution of methyl 4-(bis(4-methoxybenzyl)amino)-1-(2,6-dichloro-4-hydroxyphenyl)-6-oxo-1,6-dihydropyrimidine-5-carboxylate (1.39 g, 2.44 mmol) and sodium 2-chloro-2,2-difluoroacetate (743 mg, 4.87 mmol) in DMF (15 mL) was added K₂CO₃ (370 mg, 2.68 mmol) and the mixture stirred at 60° C. for 6 h under N₂. The reaction mixture was diluted with H₂O (30 mL) and extracted with EtOAc (3×20 mL). The combined organics were washed with brine (2×20 mL), dried (Na₂SO₄) and evaporated to dryness under reduced pressure. The residue was purified by MPLC (SiO₂, 1-50% EtOAc/PE) to give the title compound as a brown solid (657 mg, 43.5%). LCMS m/z=620 [M+H]⁺.

### Step 2. Synthesis of methyl 4-amino-1-(2,6-dichloro-4-(difluoromethoxy)phenyl)-6-oxo-1,6-dihydropyrimidine-5-carboxylate

A mixture of methyl 4-(bis(4-methoxybenzyl)amino)-1-(2,6-dichloro-4-(difluoromethoxy)phenyl)-6-oxo-1,6-dihydropyrimidine-5-carboxylate (657 mg, 1.06 mmol) in TFA (5 mL) was stirred at 80° C. for 0.5 h. The reaction mixture was concentrated under reduced pressure and the pH of the residue was adjusted with NaHCO₃ to pH 7-8. The mixture was diluted with H₂O (15 mL) and extracted with EtOAc (3×8 mL). The combined extracts were dried (Na₂SO₄) and evaporated to dryness in vacuo. The residue was purified by MPLC (SiO₂, 1-33% EtOAc/PE) to give the title compound as a brown solid (381 mg, 94.7%).

### Intermediate B46. (S)-methyl 4-amino-1-(2-chloro-4-(difluoromethyl)-6-methylphenyl)-6-oxo-1,6-dihydropyrimidine-5-carboxylate

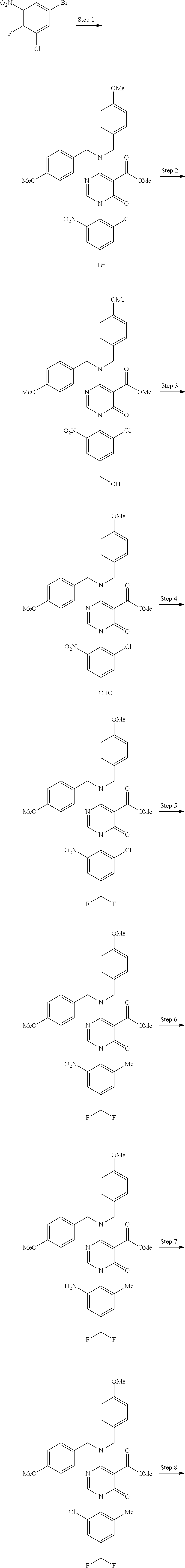

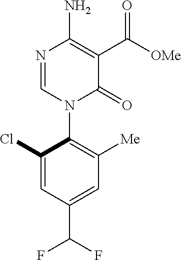

### Step 1. Synthesis of methyl 4-(bis(4-methoxybenzyl)amino)-1-(4-bromo-2-chloro-6-nitrophenyl)-6-oxo-1,6-dihydropyrimidine-5-carboxylate

To a solution of 5-bromo-1-chloro-2-fluoro-3-nitrobenzene (5 g, 19.65 mmol) and methyl 4-(bis(4-methoxybenzyl)amino)-6-oxo-1,6-dihydropyrimidine-5-carboxylate (Intermediate B1, 8.05 g, 19.65 mmol) in DMSO (15 mL) was added DIPEA (5.08 g, 39.3 mmol) and the mixture stirred at 100° C. for 12 h. The reaction mixture was concentrated under reduced pressure. The residue was diluted with water (20 mL) and extracted with EtOAc (3×20 mL), dried (Na₂SO₄) and concentrated under reduced pressure. The residue was purified by column chromatography on silica gel (0-50% EtOAc/PE) to give the title compound as a brown oil (12.5 g, 98%). LCMS m/z=645 [M+H]⁺.

### Step 2. Synthesis of methyl 4-(bis(4-methoxybenzyl)amino)-1-(2-chloro-4-(hydroxymethyl)-6-nitrophenyl)-6-oxo-1,6-dihydropyrimidine-5-carboxylate

To a solution of methyl 4-(bis(4-methoxybenzyl)amino)-1-(4-bromo-2-chloro-6-nitrophenyl)-6-oxo-1,6-dihydropyrimidine-5-carboxylate (Step 1, 12.5 g, 19.4 mmol) and tributylstannylmethanol (12.47 g, 38.8 mmol) in dioxane (30 mL) was added Pd(PPh₃)₄ (1.12 g, 0.97 mmol) and the mixture was stirred at 100° C. for 2 h under N₂. The reaction mixture was quenched by addition saturated aqueous KF (20 mL) at 0° C. and then extracted with EtOAc (3×20 mL). The combined organics were dried (Na₂SO₄) and concentrated under reduced pressure and the residue purified by column chromatography on silica gel (0-50% EtOAc/PE) to give the title compound as a brown solid (5.2 g, 45%). LCMS m/z=595 [M+H]⁺.

### Step 3. Synthesis of methyl 4-(bis(4-methoxybenzyl)amino)-1-(2-chloro-4-formyl-6-nitrophenyl)-6-oxo-1,6-dihydropyrimidine-5-carboxylate

To a solution of methyl 4-(bis(4-methoxybenzyl)amino)-1-(2-chloro-4-(hydroxymethyl)-6-nitrophenyl)-6-oxo-1,6-dihydropyrimidine-5-carboxylate (Step 2, 5.2 g, 8.74 mmol) in DCM (2 mL) was added Dess-Martin periodinane (5.56 g, 13.11 mmol) at 0° C. and the mixture was stirred at 25° C. for 1 h. The reaction mixture was quenched by addition saturated aqueous NaHCO₃ (20 mL) at 0° C. and extracted with EtOAc (3×20 mL). The combined organics were dried (Na₂SO₄) and concentrated under reduced pressure. The residue was purified by column chromatography on silica gel (0-50% EtOAc/PE) to give the title compound as a yellow solid (3.6 g, 69%). LCMS m/z=593 [M+H]⁺.

### Step 4. Synthesis of methyl 4-(bis(4-methoxybenzyl)amino)-1-(2-chloro-4-(difluoromethyl)-6-nitrophenyl)-6-oxo-1,6-dihydropyrimidine-5-carboxylate

To a solution of methyl 4-(bis(4-methoxybenzyl)amino)-1-(2-chloro-4-formyl-6-nitrophenyl)-6-oxo-1,6-dihydropyrimidine-5-carboxylate (3.6 g, 6.07 mmol) in DCM (3 mL) was added DAST (4.89 g, 30.36 mmol) at −78° C. and the mixture stirred at 25° C. for 3 h under N₂. The reaction mixture was quenched by addition saturated aqueous NaHCO₃ (15 mL) at 0° C. and extracted with DCM (3×15 mL). The combined organics were dried (Na₂SO₄) and concentrated under reduced pressure and the residue purified by column chromatography on silica gel (0-50% EtOAc/PE) to give the title compound as a yellow solid (2.64 g, 71%). LCMS m/z=615 [M+H]⁺.

### Step 5. Synthesis of methyl 4-(bis(4-methoxybenzyl)amino)-1-(4-(difluoromethyl)-2-methyl-6-nitrophenyl)-6-oxo-1,6-dihydropyrimidine-5-carboxylate

To a solution of methyl 4-(bis(4-methoxybenzyl)amino)-1-(2-chloro-4-(difluoromethyl)-6-nitrophenyl)-6-oxo-1,6-dihydropyrimidine-5-carboxylate (Step 4, 2.6 g, 4.23 mmol) and 2,4,6-trimethyl-1,3,5,2,4,6-trioxatriborinane (2.12 g, 8.46 mmol) in dioxane (15 mL) was added XPHOS-PD-G2 (333 mg, 0.423 mmol) and K₃PO₄ (1.79 g, 8.46 mmol) and the mixture stirred at 80° C. for 2 h under N₂. The reaction mixture was concentrated under reduced pressure. The residue was diluted with H₂O (15 mL) and extracted with EtOAc (3×15 mL), dried (Na₂SO₄) and concentrated under reduced pressure and the residue purified by column chromatography on silica gel (0-50% EtOAc/PE) to give the title compound as a white solid (2.2 g, 87%). LCMS m/z=595 [M+H]⁺.

### Step 6. Synthesis of methyl 1-(2-amino-4-(difluoromethyl)-6-methylphenyl)-4-(bis(4-methoxybenzyl)amino)-6-oxo-1,6-dihydropyrimidine-5-carboxylate

To a solution of methyl 4-(bis(4-methoxybenzyl)amino)-1-(4-(difluoromethyl)-2-methyl-6-nitrophenyl)-6-oxo-1,6-dihydropyrimidine-5-carboxylate (Step 5, 2.1 g, 3.53 mmol) in MeOH (15 mL) was added Pd/C (2 g, 3.53 mmol, 10% purity) and the mixture stirred at 25° C. for 1 h under H₂ (15 Psi). The reaction mixture was filtered and the filtrate concentrated under reduced pressure to give the title compound as a yellow solid (1.36 g, 68%). LCMS m/z=564 [M+H]⁺.

### Step 7. Synthesis of methyl 4-(bis(4-methoxybenzyl)amino)-1-(2-chloro-4-(difluoromethyl)-6-methylphenyl)-6-oxo-1,6-dihydropyrimidine-5-carboxylate

To a solution of methyl 1-(2-amino-4-(difluoromethyl)-6-methylphenyl)-4-(bis(4-methoxybenzyl)amino)-6-oxo-1,6-dihydropyrimidine-5-carboxylate (Step 6, 1.36 g, 2.41 mmol) in MeCN (10 mL) was added CuCl (477 mg, 4.82 mmol), CuCl₂ (972 mg, 7.23 mmol) and isopentyl nitrite (847 mg, 7.23 mmol) and the mixture stirred at 25° C. for 1 h under N₂. The reaction mixture was filtered and the filtrate concentrated under reduced pressure and the residue purified by column chromatography on silica gel (0-50% EtOAc/PE) to give the title compound as a light yellow solid (1 g, 71%). LCMS m/z=584 [M+H]⁺.

### Step 8. Synthesis of (S)-methyl 4-amino-1-(2-chloro-4-(difluoromethyl)-6-methylphenyl)-6-oxo-1,6-dihydropyrimidine-5-carboxylate

A mixture of methyl 4-(bis(4-methoxybenzyl)amino)-1-(2-chloro-4-(difluoromethyl)-6-methylphenyl)-6-oxo-1,6-dihydropyrimidine-5-carboxylate (Step 7, 1 g, 1.71 mmol) in TFA (5 mL) was stirred at 80° C. for 1 h. The reaction mixture was quenched by addition saturated aqueous NaHCO₃ (10 mL) at 0° C. and extracted with EtOAc (3×10 mL). The combined organics were dried (Na₂SO₄) and concentrated under reduced pressure to give racemic methyl 4-amino-1-(2-chloro-4-(difluoromethyl)-6-methylphenyl)-6-oxo-1,6-dihydropyrimidine-5-carboxylate as a yellow solid (170 mg, 29%). The racemate was separated by SFC (DAICEL CHIRALPAK AS; 250×30 mm, 10 μm); 23% MeOH (0.1% NH₄OH)) to afford:

Peak 1, Intermediate B46. (S)-methyl 4-amino-1-(2-chloro-4-(difluoromethyl)-6-methylphenyl)-6-oxo-1,6-dihydropyrimidine-5-carboxylate (white solid, 407 mg, 23%). LCMS m/z=344 [M+H]⁺.

### Intermediate B47. Synthesis of methyl 4-amino-1-((S)-2-chloro-6-methyl-4-(trifluoromethyl)phenyl)-6-oxo-1,6-dihydropyrimidine-5-carboxylate

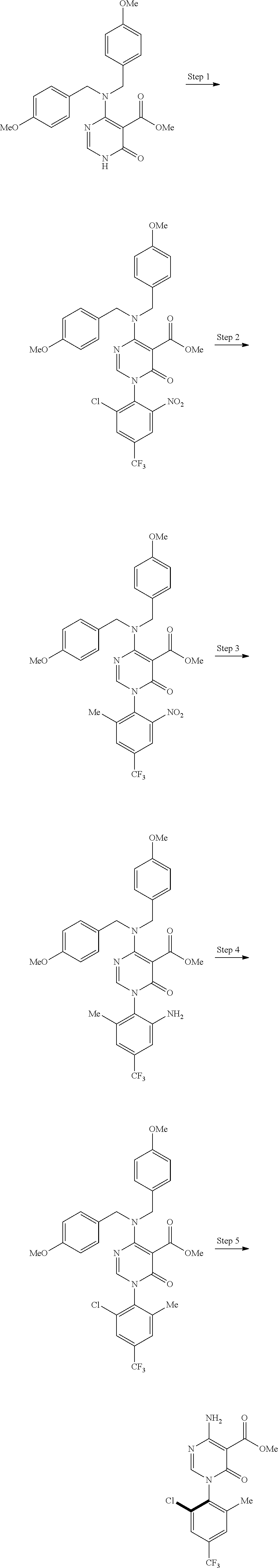

### Step 1. Synthesis of methyl 4-(bis(4-methoxybenzyl)amino)-1-(2-chloro-6-nitro-4-(trifluoromethyl)phenyl)-6-oxo-1,6-dihydropyrimidine-5-carboxylate

K₂CO₃ (5.67 g, 41.1 mmol) was added to the mixture of methyl 4-(bis(4-methoxybenzyl)amino)-6-oxo-1,6-dihydropyrimidine-5-carboxylate (Intermediate B1, 8.41 g, 20.53 mmol) and 1-chloro-2-fluoro-3-nitro-5-(trifluoromethyl)benzene (5 g, 20.53 mmol, 3.11 mL) in DMF (50 mL) was added and the mixture was stirred at 80° C. for 1 h. The mixture was diluted with H₂O (200 mL), filtered and extracted with EtOAc (3×200 mL) to give the title compound as a yellow solid (12.8 g, 98%). LCMS m/z=633 [M+H]⁺.

### Step 2. Synthesis of methyl 4-(bis(4-methoxybenzyl)amino)-1-(2-methyl-6-nitro-4-(trifluoromethyl)phenyl)-6-oxo-1,6-dihydropyrimidine-5-carboxylate

To a solution of methyl 4-(bis(4-methoxybenzyl)amino)-1-(2-chloro-6-nitro-4-(trifluoromethyl)phenyl)-6-oxo-1,6-dihydropyrimidine-5-carboxylate (Step 1, 7 g, 11.1 mmol) in dioxane (30 mL) was added 2,4,6-trimethyl-1,3,5,2,4,6-trioxatriborinane (8.33 g, 33.2 mmol), XPHOS-PD-G2 (870 mg, 1.11 mmol) and K₃PO₄ (4.69 g, 22.1 mmol) and the mixture degassed and purged with N₂ (3×) and stirred at 80° C. for 12 h under N₂. The reaction mixture was filtered and the filtrate concentrated under reduced pressure and the residue purified by column chromatography (SiO₂, 0-50% EtOAc/PE) to give the title compound as a yellow oil (5 g73%). LCMS m/z=613 [M+H]⁺.

### Step 3. Synthesis of methyl 1-(2-amino-6-methyl-4-(trifluoromethyl)phenyl)-4-(bis(4-methoxybenzyl)amino)-6-oxo-1,6-dihydropyrimidine-5-carboxylate

Fe (7.29 g, 130 mmol) and NH₄Cl (6.99 g, 130 mmol) were added to a solution of methyl 4-(bis(4-methoxybenzyl)amino)-1-(2-methyl-6-nitro-4-(trifluoromethyl)phenyl)-6-oxo-1,6-dihydropyrimidine-5-carboxylate (Step 2, 8 g, 13.1 mmol) in THF (30 mL), EtOH (30 mL) and H₂O (10 mL) and the mixture stirred at 80° C. for 2 h. The reaction mixture was filtered and the filtrate evaporated to dryness and the residue purified by column chromatography on silica gel (0-50% EtOAc/PE) to give the title compound as a yellow solid (4.5 g, 59%). LCMS m/z=583 [M+H]⁺.

### Step 4. Synthesis of methyl 4-(bis(4-methoxybenzyl)amino)-1-(2-chloro-6-methyl-4-(trifluoromethyl)phenyl)-6-oxo-1,6-dihydropyrimidine-5-carboxylate

To a solution of methyl 1-(2-amino-6-methyl-4-(trifluoromethyl)phenyl)-4-(bis(4-methoxybenzyl)amino)-6-oxo-1,6-dihydropyrimidine-5-carboxylate (Step 3, 4.3 g, 7.38 mmol) in MeCN (40 mL) was added CuCl (1.46 g, 14.8 mmol), CuCl₂ (2.98 g, 22.1 mmol) and isopentyl nitrite (2.59 g, 22.1 mmol) and the mixture stirred at 25° C. for 1 h. The reaction mixture was diluted with H₂O (20 mL) and extracted with EtOAc (4×20 mL). The combined extracts were concentrated under reduced pressure and the residue purified by column chromatography on silica gel (0-50% EtOAc/PE) to give the title compound as a yellow oil (3.5 g, 78%). LCMS m/z=602 [M+H]⁺.

### Step 5. Synthesis of methyl 4-amino-1-((S)-2-chloro-6-methyl-4-(trifluoromethyl)phenyl)-6-oxo-1,6-dihydropyrimidine-5-carboxylate

A solution of methyl 4-(bis(4-methoxybenzyl)amino)-1-(2-chloro-6-methyl-4-(trifluoromethyl)phenyl)-6-oxo-1,6-dihydropyrimidine-5-carboxylate (Step 4, 1.5 g, 2.49 mmol) in TFA (2 mL) was stirred at 80° C. for 1 h. The reaction mixture was concentrated under reduced pressure and the residue was purified by column chromatography on silica gel (0-50% EtOAc/PE) followed by chiral SFC (DAICEL CHIRALCEL OD, 250×30 mm, 10 μm; 15% IPA (0.1% NH₄OH) in CO₂) to afford:

Peak 2, Intermediate B47. methyl 4-amino-1-((S)-2-chloro-6-methyl-4-(trifluoromethyl)phenyl)-6-oxo-1,6-dihydropyrimidine-5-carboxylate (white solid, 400 mg, 40%). LCMS m/z=362 [M+H]⁺.

### Intermediate B48. Synthesis of methyl 4-amino-1-((S)-2-chloro-6-(difluoromethyl)phenyl)-6-oxo-1,6-dihydropyrimidine-5-carboxylate

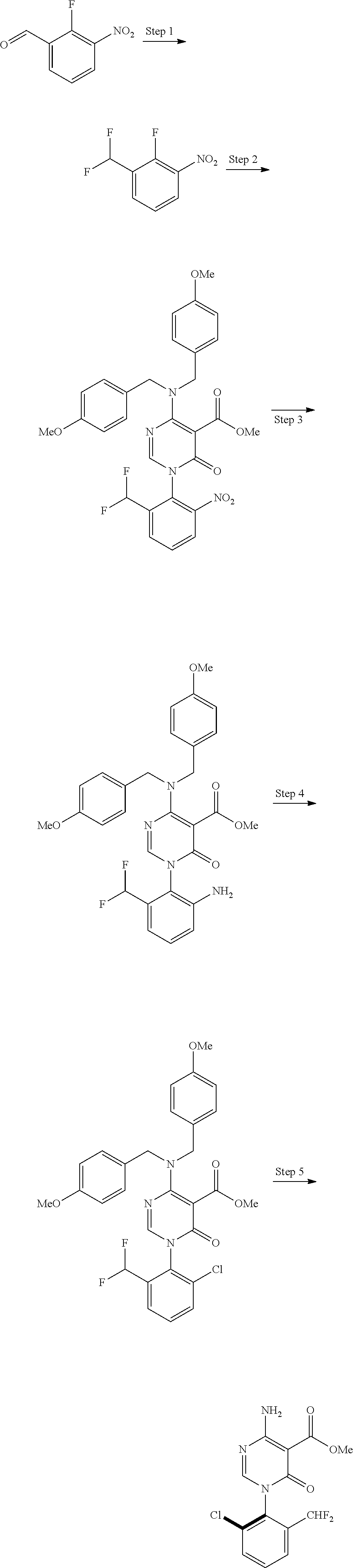

### Step 1. Synthesis of 1-(difluoromethyl)-2-fluoro-3-nitrobenzene

To the mixture of 2-fluoro-3-nitrobenzaldehyde (5 g, 29.6 mmol) in DCM (50 mL) was added DAST (14.30 g, 88.7 mmol) at −78° C. under N₂ and the mixture stirred at −78° C. for 0.5 h for by stirring at 20° C. for 0.5 h under N₂. The reaction mixture was partitioned between DCM (50 mL) and sat. aq. NaHCO₃ (50 mL). The aqueous phase was extracted with DCM (3×50 mL) and the combined organics were dried (Na₂SO₄) and concentrated under reduced. The residue was purified by MPLC (SiO₂, 0-16% EtOAc/PE) to give the title compound as a yellow oil (5 g, 88%). ¹H NMR (400 MHz, CDCl₃) δ: 8.24 (t, 1H), 7.97 (br t, 1H), 7.49 (t, 1H), 7.15-6.88 (m, 1H).

### Step 2. Synthesis of methyl 4-(bis(4-methoxybenzyl)amino)-1-(2-(difluoromethyl)-6-nitrophenyl)-6-oxo-1,6-dihydropyrimidine-5-carboxylate

A mixture of methyl 4-(bis(4-methoxybenzyl)amino)-6-oxo-1,6-dihydropyrimidine-5-carboxylate (Intermediate B1, 3.32 g, 8.11 mmol) and 1-(difluoromethyl)-2-fluoro-3-nitrobenzene (Step 1, 3.1 g, 16.2 mmol) in DMSO (40 mL) was added DIPEA (2.10 g, 16.2 mmol) and the mixture was stirred at 100° C. for 12 h. The reaction mixture was filtered and the filtrate was concentrated under reduced pressure and the residue purified by column chromatography on silica gel (0-33% EtOAc/PE) to give the title compound as a white solid (4.7 g, 99%). LCMS m/z=581 [M+H]⁺.

### Step 3. Synthesis of methyl 1-(2-amino-6-(difluoromethyl)phenyl)-4-(bis(4-methoxybenzyl)amino)-6-oxo-1,6-dihydropyrimidine-5-carboxylate

To a solution of methyl 4-(bis(4-methoxybenzyl)amino)-1-(2-(difluoromethyl)-6-nitrophenyl)-6-oxo-1,6-dihydropyrimidine-5-carboxylate (Step 2, 4.7 g, 8.10 mmol) in MeOH (2 mL) and THF (2 mL) was added Pd/C (50 mg, 8.10 mmol, 10% purity) and the mixture stirred at 25° C. for 1 h under H₂ (15 Psi). The reaction mixture was filtered and the filtrate concentrated under reduced pressure to give the title compound as a light yellow solid (4.8 g, crude). LCMS m/z=551 [M+H]⁺.

### Step 4. Synthesis of methyl 4-(bis(4-methoxybenzyl)amino)-1-(2-chloro-6-(difluoromethyl)phenyl)-6-oxo-1,6-dihydropyrimidine-5-carboxylate

To a solution of methyl 1-(2-amino-6-(difluoromethyl)phenyl)-4-(bis(4-methoxybenzyl)amino)-6-oxo-1,6-dihydropyrimidine-5-carboxylate (Step 3, 4.8 g, 8.72 mmol) in MeCN (30 mL) was added CuCl (1.73 g, 17.44 mmol), CuCl₂ (3.52 g, 26.2 mmol), and a solution of isopentyl nitrite (3.06 g, 26.2 mmol) in MeCN (5 mL) and the mixture was stirred at 20° C. for 1 h under N₂. The reaction mixture was concentrated under reduced pressure and the residue diluted with H₂O (20 mL) and extracted with EtOAc (3×20 mL). The combined extracts were dried (Na₂SO₄) and concentrated under reduced pressure and the residue purified by column chromatography on silica gel (0-50% EtOAc/PE) to give the title compound as a yellow solid (3.13 g, 63%). LCMS m/z=570 [M+H]⁺.

### Step 5. Synthesis of methyl 4-amino-1-((S)-2-chloro-6-(difluoromethyl)phenyl)-6-oxo-1,6-dihydropyrimidine-5-carboxylate

A mixture of methyl 4-(bis(4-methoxybenzyl)amino)-1-(2-chloro-6-(difluoromethyl)phenyl)-6-oxo-1,6-dihydropyrimidine-5-carboxylate (Step 4, 3.13 g, 5.49 mmol) in TFA (30 mL) was stirred at 80° C. for 1 hr. The reaction mixture was filtered and the filtrate was concentrated under reduced pressure and the residue was purified by column chromatography on silica gel (0-100% EtOAc/PE) to give racemic methyl 4-amino-1-(2-chloro-6-(difluoromethyl)phenyl)-6-oxo-1,6-dihydropyrimidine-5-carboxylate as a yellow solid (1.76 g, 97%). The racemic material was separated by chiral SFC (DAICEL CHIRALPAK AD, 250×30 mm, 10 μm); 30% IPA (0.1% NH₄OH)) to afford:

### Intermediate B48, Peak 1; methyl 4-amino-1-((S)-2-chloro-6-(difluoromethyl)phenyl)-6-oxo-1,6-dihydropyrimidine-5-carboxylate (white solid, 445 mg, 44%). LCMS m/z=330 [M+H]⁺

### Intermediate B49. methyl 4-amino-1-(2,6-dichloro-4-(2-methoxyethyl)phenyl)-6-oxo-1,6-dihydropyrimidine-5-carboxylate

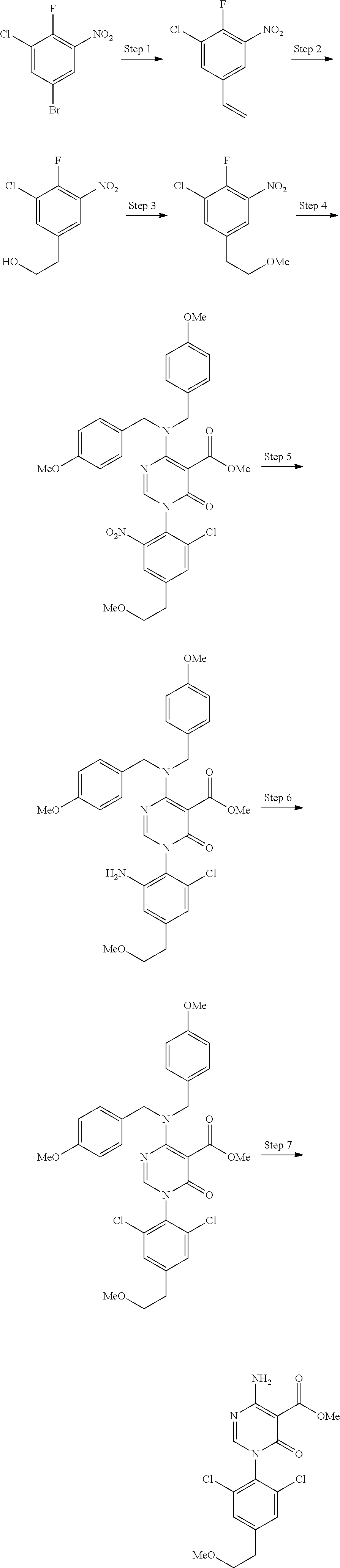

### Step 1. Synthesis of 1-chloro-2-fluoro-3-nitro-5-vinylbenzene

To the mixture of 5-bromo-1-chloro-2-fluoro-3-nitrobenzene (8 g, 31.4 mmol) and 4,4,5,5-tetramethyl-2-vinyl-1,3,2-dioxaborolane (7.26 g, 47.2 mmol) in dioxane (80 mL) and H₂O (10 mL) was added K₂CO₃ (8.69 g, 62.8 mmol) and Pd(dppf)Cl₂ (2.30 g, 3.14 mmol) ans the mixture stirred at 100° C. for 2 h under N₂. The solid were removed by filtration and the filtrate concentrated under reduced pressure and the residue purified by MPLC (SiO₂, 0-16% EtOAc/PE) to give the title compound as a yellow oil (4.2 g, 66%). ¹H NMR (400 MHz, CDCl₃) δ: 7.87 (dd, 1H), 7.64 (dd, 1H), 6.57 (dd, 1H), 5.75 (d, 1H), 5.41 (d, 1H).

### Step 2. Synthesis of 2-(3-chloro-4-fluoro-5-nitrophenyl) ethan-1-ol

To the mixture of 1-chloro-2-fluoro-3-nitro-5-vinylbenzene (Step 2, 2.1 g, 10.42 mmol) in THF (20 mL) was added 9-BBN (0.5 M, 62.5 mL) at 0° C. and the mixture stirred at 20° C. for 12 h under N₂. The pH of the reaction mixture was adjusted to pH 10 by the addition of NaOH (1M, 36.46 mL, 3.5 eq) at 0° C. To this was added H₂O₂ (10.87 g, 0.096 mmol, 9.21 mL, 30% purity) under N₂ and the resulting mixture stirred at 20° C. for 2 h under N₂. The mixture was added to sat. aq. Na₂SO₃ (50 mL) at 0° C. and extracted with EtOAc (3×10 mL). The combined organics were dried (Na₂SO₄) and concentrated under reduced pressure and the residue purified by MPLC (SiO₂, 0-50% EtOAc/PE) to give the title compound as a yellow oil (700 mg, 30%). ¹H NMR (400 MHz, CDCl₃) δ: 7.77 (dd, 1H), 7.54 (dd, 1H), 3.85 (t, 2H), 2.83 (t, 2H).

### Step 3. Synthesis of 1-chloro-2-fluoro-5-(2-methoxyethyl)-3-nitrobenzene

To a mixture of 2-(3-chloro-4-fluoro-5-nitrophenyl) ethan-1-ol (1.3 g, 5.92 mmol) in CHCI3 (15 mL) was added trimethyloxonium tetrafluoroborate (3.5 g, 23.7 mmol) and the stirred at 40° C. for 1 h. The reaction mixture was partitioned between EtOAc (20 mL) and sat. aq. NaHCO₃ (15 mL) and the aqueous phase was extracted with EtOAc (3×20 mL). The combined organics were dried (Na₂SO₄) and concentrated under reduced pressure to give a residue which was purified by MPLC (SiO₂, 0-33% EtOAc/PE) to give the title compound as a yellow oil (700 mg, 50%). ¹H NMR (400 MHz, CDCl₃) δ: 7.86 (dd, 1H), 7.63 (td, 1H), 3.65 (t, 2H), 3.38 (s, 3H), 2.95-2.87 (m, 2H).

### Step 4. Synthesis of methyl 4-(bis(4-methoxybenzyl)amino)-1-(2-chloro-4-(2-methoxyethyl)-6-nitrophenyl)-6-oxo-1,6-dihydropyrimidine-5-carboxylate

To the mixture of methyl 4-(bis(4-methoxybenzyl)amino)-6-oxo-1,6-dihydropyrimidine-5-carboxylate (Intermediate B1, 1 g, 2.44 mmol) and 1-chloro-2-fluoro-5-(2-methoxyethyl)-3-nitrobenzene (Step 3, 570 mg, 2.44 mmol) in DMF (6 mL) was added K₂CO₃ (675 mg, 4.88 mmol) and the mixture stirred at 80° C. for 1 h. The mixture was filtered and the filtrate evaporated to dryness in vacuo and the residue partitioned between EtOAc (20 mL) and H₂O (15 mL) and the aqueous was extracted with EtOAc (3×20 mL). The combined organics were dried (Na₂SO₄) and concentrated under reduced pressure and the residue purified by MPLC (SiO₂, 0-66% EtOAc/PE) to give the title compound as a yellow oil (1.1 g, 1.77 mmol, 72.29% yield) as a yellow oil. LCMS m/z=623 [M+H]⁺.

### Step 5. Synthesis of methyl 1-(2-amino-6-chloro-4-(2-methoxyethyl)phenyl)-4-(bis(4-methoxybenzyl)amino)-6-oxo-1,6-dihydropyrimidine-5-carboxylate

Fe (968 mg, 17.3 mmol) and NH₄Cl (927 mg, 17.3 mmol) were added to a solution of methyl 4-(bis(4-methoxybenzyl)amino)-1-(2-chloro-4-(2-methoxyethyl)-6-nitrophenyl)-6-oxo-1,6-dihydropyrimidine-5-carboxylate (Step 4, 1.08 g, 1.73 mmol) in THF (8 mL), EtOH (8 mL) and H₂O (3 mL) and stirred at 70° C. for 2 h. The mixture was filtered and the filtrate concentrated under reduced pressure. The residue was treated with DCM (10 mL), filtered and concentrated under reduced pressure to give the title compound as a yellow oil (1.0 g, crude). LCMS m/z=593 [M+H]⁺.

### Step 6. Synthesis of methyl 4-(bis(4-methoxybenzyl)amino)-1-(2,6-dichloro-4-(2-methoxyethyl)phenyl)-6-oxo-1,6-dihydropyrimidine-5-carboxylate

To a solution of methyl 1-(2-amino-6-chloro-4-(2-methoxyethyl)phenyl)-4-(bis(4-methoxybenzyl)amino)-6-oxo-1,6-dihydropyrimidine-5-carboxylate (Step 5, 950 mg, 1.60 mmol) in MeCN (15 mL) was added CuCl₂ (646 mg, 4.81 mmol), CuCl (317 mg, 3.20 mmol). To this was added dropwise a solution of isopentyl nitrite (563 mg, 4.81 mmol) in MeCN (2 mL) and the mixture stirred at 20° C. for 1 h. The reaction mixture was partitioned between EtOAc (20 mL) and H₂O (15 mL) and the aqueous phase extracted with EtOAc (3×20 mL). The combined organics were dried (Na₂SO₄) and concentrated under reduced pressure and the residue purified by MPLC (SiO₂, 0-100% EtOAc/PE) to give the title compound as a yellow oil (700 mg, 71%). LCMS m/z=612 [M+H]⁺.

### Step 7. Synthesis of methyl 4-amino-1-(2,6-dichloro-4-(2-methoxyethyl)phenyl)-6-oxo-1,6-dihydropyrimidine-5-carboxylate

A solution of methyl 4-(bis(4-methoxybenzyl)amino)-1-(2,6-dichloro-4-(2-methoxyethyl)phenyl)-6-oxo-1,6-dihydropyrimidine-5-carboxylate (680 mg, 1.11 mmol) in TFA (5 mL) was stirred at 80° C. for 0.5 hr. The mixture was concentrated under reduced pressure and the residue partitioned between EtOAc (20 mL) and sat. aq. NaHCO₃ (15 mL).

The aqueous phase was extracted with EtOAc (3×20 mL) and the combined organics dried (Na₂SO₄), concentrated under reduced pressure and the residue purified by MPLC (SiO₂, 0-100% EtOAc/PE) to give the title compound as a brown solid (350 mg, 84%). LCMS m/z=372 [M+H]⁺.

### Intermediate B50. methyl 4-amino-1-(4-fluoro-2,6-dimethylphenyl)-6-oxo-1,6-dihydropyrimidine-5-carboxylate

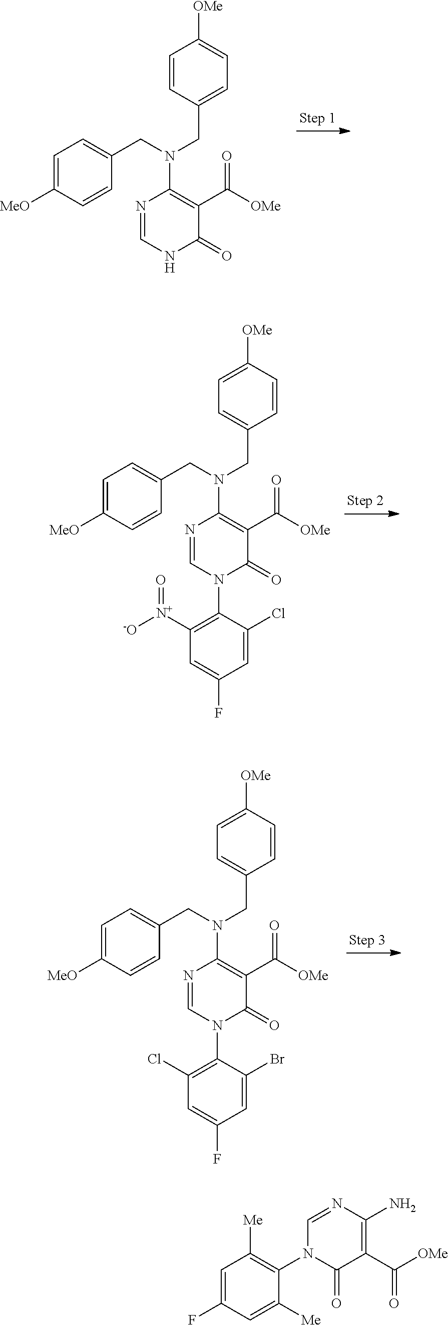

### Step 1. Synthesis of methyl 4-(bis(4-methoxybenzyl)amino)-1-(2-chloro-4-fluoro-6-nitrophenyl)-6-oxo-1,6-dihydropyrimidine-5-carboxylate

To a solution of 1-chloro-2,5-difluoro-3-nitrobenzene (1.156 g, 6.0 mmol) in anhydrous MeCN (20 mL) were added methyl 4-(bis(4-methoxybenzyl)amino)-6-oxo-1,6-dihydropyrimidine-5-carboxylate (Intermediate B1, 2.71 g, 6.6 mmol) and K₂CO₃ (1.70 g, 12.0 mmol) and the reaction heated to 80° C. overnight. The reaction was poured into 200 mL water and extracted with EtOAc (4×75 mL). The combined organics were washed twice with water, brine, dried (MgSO₄) and concentrated by rotary evaporation. Flash chromatography (ISCO 40 g silica, 0-80% EtOAc/hex) provided the title compound as a yellow foam (3.286 g, 94%). LCMS m/z=583 [M+H]⁺.

### Step 2. Synthesis of methyl 4-(bis(4-methoxybenzyl)amino)-1-(2-bromo-6-chloro-4-fluorophenyl)-6-oxo-1,6-dihydropyrimidine-5-carboxylate

A suspension of methyl 4-(bis(4-methoxybenzyl)amino)-1-(2-chloro-4-fluoro-6-nitrophenyl)-6-oxo-1,6-dihydropyrimidine-5-carboxylate (Step 1, 3.286 g, 5.65 mmol) and iron powder (2.02 g, 33.9 mmol) in MeOH (33 mL)/glacial acetic acid (6 mL) was heated to 50° C. overnight. The reaction was then cooled and residual iron was removed with a magnet. The mixture was poured into 250 mL water and stirred at room temp for 15 minutes to generate a precipitate. The precipitate was collected by filtration, washing with 150 mL water and dried to provide a yellow powder which was used without purification. LCMS m/z=575 [M+Na]⁺.

A solution of the crude methyl 1-(2-amino-6-chloro-4-fluorophenyl)-4-(bis(4-methoxybenzyl)amino)-6-oxo-1,6-dihydropyrimidine-5-carboxylate (5.65 mmol) in MeCN (55 mL) and 48% aqueous HBr (1.60 mL, 14.1 mmol) was cooled on ice. Sodium nitrite (494 mg, 6.78 mmol) was added and the mixture was stirred at 0° C. for 75 minutes before CuBr (979 mg, 6.78 mmol) was added at 0° C. The reaction mixture was stirred for 3 h leaving in the ice bath to gradually warm to RT. The mixture was filtered to remove copper salts, washing w/EtOAc and MeOH. The combined organics were concentrated by rotary evaporation. The crude residue was dissolved in EtOAc (150 mL) and dilute aqueous HCl (200 mL). The aqueous phase was extracted with EtOAc (2×50 mL). The combined organics were washed with saturated aqueous NaHCO₃, water, brine, dried (MgSO₄) and evaporated to dryness. The residue was purified by flash chromatography (ISCO 40 g silica, 0-100% EtOAc/hex) to afford the title compound as a light yellow foam (1.641 g, 47% yield over 2 steps). The material was carried on to the next step without further purification. LCMS m/z=616 [M+H]⁺.

### Step 3. Synthesis of methyl 4-amino-1-(4-fluoro-2,6-dimethylphenyl)-6-oxo-1,6-dihydropyrimidine-5-carboxylate

To a suspension of methyl 4-(bis(4-methoxybenzyl)amino)-1-(2-bromo-6-chloro-4-fluorophenyl)-6-oxo-1,6-dihydropyrimidine-5-carboxylate (Step 2, 1.64 g, 2.65 mmol), K₃PO₄ (1.703 g, 7.95 mmol), and Xphos Pd G3 (223 mg, 0.265 mmol) in dioxane (10 mL) was added trimethylboroxine solution (50 wt % in THF, 1.90 mL, 6.63 mmol) and the mixture was purged with N₂ for 10 min and heated to 80° C. for 16 h. The reaction was filtered through Celite and washed with EtOAc. The combined organics were evaporated to dryness and the residue purified by flash chromatography (ISCO 40 g silica, 0-100% EtOAc/hex) to give a viscous amber oil. The oil was dissolved in TFA (6 mL) and heated to 80° C. for 1 h, cooled, poured into saturated aqueous NaHCO₃ and extracted with EtOAc (3×30 mL). The combined organics were washed with water, brine, dried (MgSO₄) and concentrated by rotary evaporation. The residue was purified by flash chromatography (ISCO 24 g silica, 0-10% MeOH/DCM) to afford the title compound as a light yellow powder (767 mg, 79% yield over 2 steps). LCMS m/z=292 [M+H]⁺.

### Intermediate B51. methyl 4-amino-1-(4-(1-methoxyethyl)-2,6-dimethylphenyl)-6-oxo-1,6-dihydropyrimidine-5-carboxylate

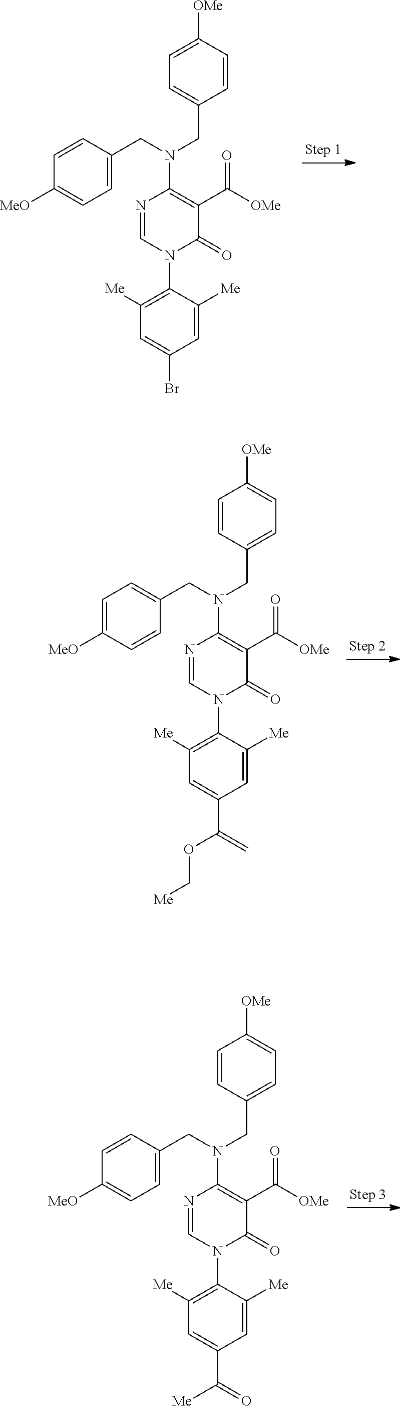 [SMILES: COC(=O)c1c(N(Cc2ccc(OC)cc2)Cc2ccc(OC)cc2)ncn(-c2c(C)cc(Br)cc2C)c1=O ; C=C(OCC)c1cc(C)c(-n2cnc(N(Cc3ccc(OC)cc3)Cc3ccc(OC)cc3)c(C(=O)OC)c2=O)c(C)c1 ; COC(=O)c1c(N(Cc2ccc(OC)cc2)Cc2ccc(OC)cc2)ncn(-c2c(C)cc(C(C)=O)cc2C)c1=O]

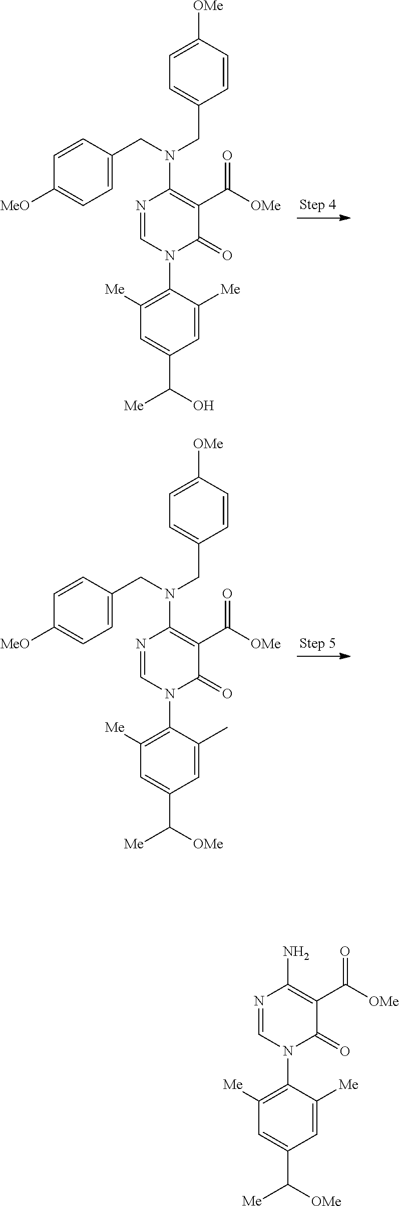

### Step 1. Synthesis of methyl 4-(bis(4-methoxybenzyl)amino)-1-(4-(1-ethoxyvinyl)-2,6-dimethylphenyl)-6-oxo-1,6-dihydropyrimidine-5-carboxylate

A mixture of methyl 4-(bis(4-methoxybenzyl)amino)-1-(4-bromo-2,6-dimethylphenyl)-6-oxo-1,6-dihydropyrimidine-5-carboxylate (Intermediate B18 Step 3, 3.5 g, 5.91 mmol), tributyl(1-ethoxyvinyl) stannane (3.20 g, 8.86 mmol), Pd(PPh₃)₄ (683 mg, 0.591 mmol) in dioxane (15 mL) was degassed and purged with N₂ (3×) and the mixture stirred at 80° C. for 12 h under N₂. The reaction mixture was quenched by addition saturated aqueous KF solution (30 mL) at 0° C. and extracted with EtOAc (3×20 mL). The combined organics were dried (Na₂SO₄) and concentrated under reduced pressure to give the title compound as a brown oil (3 g, crude). LCMS m/z=584 [M+H]⁺.

### Step 2. Synthesis of methyl 1-(4-acetyl-2,6-dimethylphenyl)-4-(bis(4-methoxybenzyl)amino)-6-oxo-1,6-dihydropyrimidine-5-carboxylate

To a solution of methyl 4-(bis(4-methoxybenzyl)amino)-1-(4-(1-ethoxyvinyl)-2,6-dimethylphenyl)-6-oxo-1,6-dihydropyrimidine-5-carboxylate (Step 1, 3 g, 5.14 mmo) in THF (10 mL) was added aqueous citric acid solution (2 M, 50 mL) and the mixture stirred at 25° C. for 2 h. The reaction mixture was diluted with H₂O (50 mL) and extracted with EtOAc (3×40 mL). The combined organics were washed with brine (2×30 mL), dried (Na₂SO₄) and concentrated under reduced pressure and the residue purified by column chromatography (SiO₂, 0-66% EtOAc/PE) to give the title compound as a yellow solid (10.2 g, crude). LCMS m/z=556 [M+H]⁺.

### Step 3. Synthesis of methyl 4-(bis(4-methoxybenzyl)amino)-1-(4-(1-hydroxyethyl)-2,6-dimethylphenyl)-6-oxo-1,6-dihydropyrimidine-5-carboxylate

To a mixture of methyl 1-(4-acetyl-2,6-dimethylphenyl)-4-(bis(4-methoxybenzyl)amino)-6-oxo-1,6-dihydropyrimidine-5-carboxylate (Step 2, 10 g, 9.00 mmol) in MeOH (100 mL) was added NaBH₄ (2.04 g, 54 mmol, 6 eq) at 0° C. and the mixture stirred at 0° C. for 20 min. The reaction mixture was quenched by addition 1M HCl (10 ml) at 0° C. basified with Na₂CO₃ aq. and extracted with EtOAc (4×50 mL). The combined organics were dried (Na₂SO₄) and concentrated under reduced pressure. The residue was purified by prep-HPLC-27 (50-80% MeCN) to give the title compound as a white solid (1 g, 20%). LCMS m/z=558 [M+H]⁺.

### Step 4. Synthesis of methyl 4-(bis(4-methoxybenzyl)amino)-1-(4-(1-methoxyethyl)-2,6-dimethylphenyl)-6-oxo-1,6-dihydropyrimidine-5-carboxylate

To a solution of methyl 4-(bis(4-methoxybenzyl)amino)-1-(4-(1-hydroxyethyl)-2,6-dimethylphenyl)-6-oxo-1,6-dihydropyrimidine-5-carboxylate (Step 3, 300 mg, 0.538 mmol) in THF (6 mL) was added NaOH (32.3 mg, 0.81 mmol) and dimethyl sulfate (679 mg, 5.38 mmol) and the mixture stirred at 25° C. for 12 h. The reaction mixture was diluted with water (30 mL) and extracted with EtOAc (3×20 mL), dried (Na₂SO₄) and concentrated under reduced pressure. The residue was purified by prep-HPLC-11 (35-65% MeCN) to give the title compound as a white solid (150 mg, 49%). LCMS m/z=572 [M+H]⁺.

### Step 5. Synthesis of methyl 4-amino-1-(4-(1-methoxyethyl)-2,6-dimethylphenyl)-6-oxo-1,6-dihydropyrimidine-5-carboxylate

A mixture of methyl 4-(bis(4-methoxybenzyl)amino)-1-(4-(1-methoxyethyl)-2,6-dimethylphenyl)-6-oxo-1,6-dihydropyrimidine-5-carboxylate (Step 4, 140 mg, 0.245 mmol) in TFA (2.5 mL) was stirred at 50° C. for 2 hr. The reaction mixture was concentrated under reduced pressure and the residue dissolved in THF and the pH adjusted to pH 9-11 with aqueous NaHCO₃ and concentrated under reduced pressure. The residue was purified by prep-HPLC-11 (10-40% MeCN) to give the title compound as a white solid (60 mg, 74%). LCMS m/z=332 [M+H]⁺.

### Intermediate B52. methyl 4-amino-1-(3,5-dichloropyridin-4-yl)-6-oxo-1,6-dihydropyrimidine-5-carboxylate

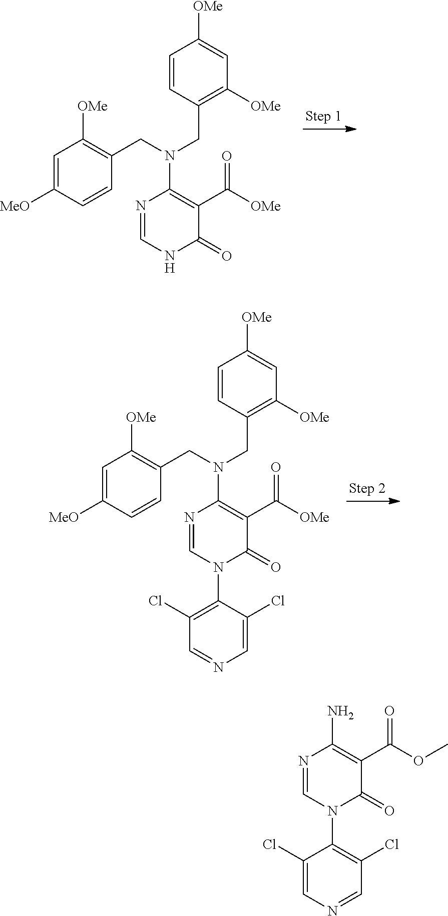 [SMILES: COC(=O)c1c(N(Cc2ccc(OC)cc2OC)Cc2ccc(OC)cc2OC)nc[nH]c1=O ; COC(=O)c1c(N(Cc2ccc(OC)cc2OC)Cc2ccc(OC)cc2OC)ncn(-c2c(Cl)cncc2Cl)c1=O ; COC(=O)c1c(N)ncn(-c2c(Cl)cncc2Cl)c1=O]

### Step 1. Synthesis of methyl 4-(bis(2,4-dimethoxybenzyl)amino)-1-(3,5-dichloropyridin-4-yl)-6-oxo-1,6-dihydropyrimidine-5-carboxylate

To a solution of methyl 4-(bis(2,4-dimethoxybenzyl)amino)-6-oxo-1,6-dihydropyrimidine-5-carboxylate (Intermediate B2, 2 g, 4.26 mmol) and 3,5-dichloro-4-fluoropyridine (1.06 g, 6.39 mmol) in DMF (10 mL) was added K₂CO₃ (1.18 g, 8.52 mmol) and the mixture stirred at 80° C. for 1 h. The reaction mixture was concentrated under reduced pressure and the residue diluted with H₂O (50 mL) and extracted with EtOAc (3×40 mL). The combined organics were washed with brine (2×30 mL), dried (Na₂SO₄) and concentrated under reduced pressure. The residue was purified by MPLC (SiO₂, 1-100% EtOAc/PE) to give the title compound as a white solid (2.3 g, 87%). LCMS m/z=615 [M+H]⁺.

### Step 2. Synthesis of methyl 4-amino-1-(3,5-dichloropyridin-4-yl)-6-oxo-1,6-dihydropyrimidine-5-carboxylate

To a solution of methyl 4-(bis(2,4-dimethoxybenzyl)amino)-1-(3,5-dichloropyridin-4-yl)-6-oxo-1,6-dihydropyrimidine-5-carboxylate (Step 1, 2.3 g, 3.74 mmol) in TFA (3 mL) and DCM (20 mL) was stirred at 25° C. for 1 h. The reaction mixture was concentrated under reduced pressure and the residue diluted with H₂O (50 mL) and extracted with EtOAc (3×40 mL). The combined organics were washed with brine (2×30 mL), dried (Na₂SO₄) and concentrated under reduced pressure. The residue was purified by MPLC (SiO₂, 1-50% EtOAc/PE) to give the title compound as a white solid (942 mg, 80% yield). LCMS m/z=315 [M+H]⁺.

### Intermediate B53. methyl 4-amino-1-(2,6-dimethyl-4-((methylsulfonyl)methyl)phenyl)-6-oxo-1,6-dihydropyrimidine-5-carboxylate

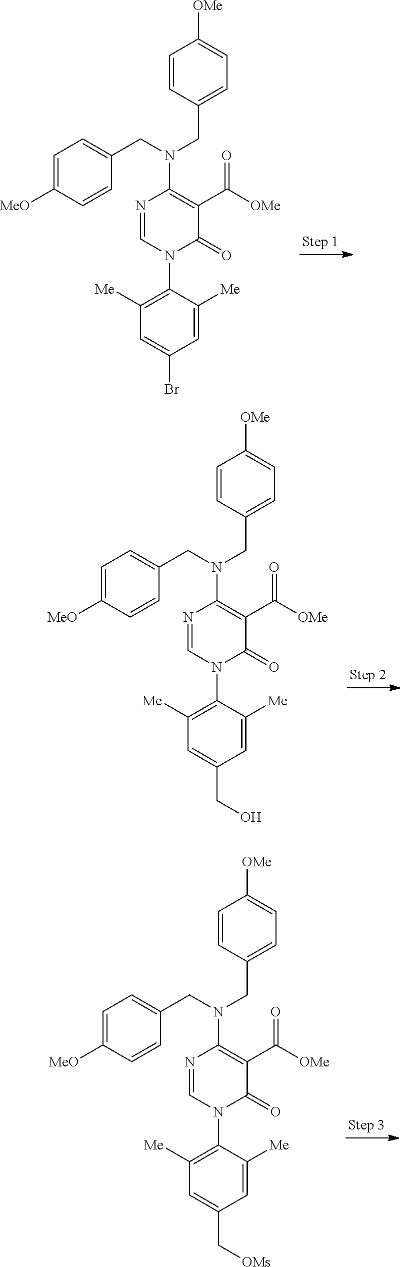

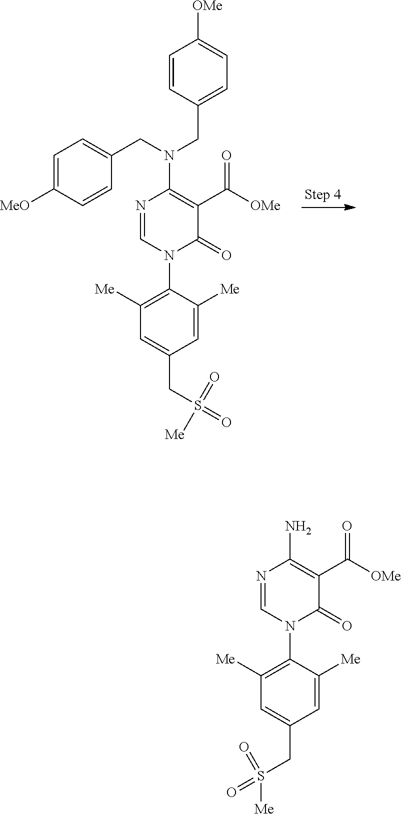 [SMILES: COC(=O)c1c(N(Cc2ccc(OC)cc2)Cc2ccc(OC)cc2)ncn(-c2c(C)cc(CS(C)(=O)=O)cc2C)c1=O ; COC(=O)c1c(N)ncn(-c2c(C)cc(CS(C)(=O)=O)cc2C)c1=O]

### Step 1. Synthesis of methyl 4-(bis(4-methoxybenzyl)amino)-1-(4-(hydroxymethyl)-2,6-dimethylphenyl)-6-oxo-1,6-dihydropyrimidine-5-carboxylate

To a mixture of methyl 4-(bis(4-methoxybenzyl)amino)-1-(4-bromo-2,6-dimethylphenyl)-6-oxo-1,6-dihydropyrimidine-5-carboxylate (Intermediate B18 Step 3, 2.00 g, 3.38 mmol) in dioxane (100 mL) was added cataCXium® A Pd G2 (226 mg, 0.338 mmol) and tributylstannylmethanol (4.34 g, 13.5 mmol) at 0° C. and the reaction mixture stirred at 110° C. for 12 h under N₂. The reaction mixture was filtered and the filtrate was concentrated under reduced pressure and the residue was purified by column chromatography (SiO₂, 0-100% EtOAc/PE) to give the title compound as a black solid (1.80 g, 98%). LCMS m/z=544 [M+H]⁺.

### Step 2. Synthesis of methyl 4-(bis(4-methoxybenzyl)amino)-1-(2,6-dimethyl-4-(((methylsulfonyl)oxy)methyl)phenyl)-6-oxo-1,6-dihydropyrimidine-5-carboxylate

To a mixture of methyl 4-(bis(4-methoxybenzyl)amino)-1-(4-(hydroxymethyl)-2,6-dimethylphenyl)-6-oxo-1,6-dihydropyrimidine-5-carboxylate (Step 1, 500 mg, 0.92 mmol) in DCM (10 mL) was added TEA (186 mg, 1.84 mmol) and MsCl (220 mg, 1.92 mmol) at 0° C. and the reaction mixture stirred at 25° C. for 30 mins. The reaction mixture was quenched with sat. aq. NaHCO₃ (2 mL) and H₂O (8 mL) and extracted with DCM (3×10 mL). The combined organics were washed with brine (10 mL) and dried (Na₂SO₄) and concentrated to give the title compound as a yellow oil (570 mg, crude) which was used without further purification.

### Step 3. Synthesis of methyl 4-(bis(4-methoxybenzyl)amino)-1-(2,6-dimethyl-4-((methylsulfonyl)methyl)phenyl)-6-oxo-1,6-dihydropyrimidine-5-carboxylate

To a solution of methyl 4-(bis(4-methoxybenzyl)amino)-1-(2,6-dimethyl-4-(((methylsulfonyl)oxy)methyl)phenyl)-6-oxo-1,6-dihydropyrimidine-5-carboxylate (Step 2, 200 mg, 0.322 mmol) in DMF (1 mL) was added sodium methanesulfinate (131 mg, 1.29 mmol) and the mixture stirred at 100° C. for 1.5 h. The reaction mixture was extracted with EtOAc (3×10 mL). The combined organics were dried (Na₂SO₄) and concentrated under reduced pressure to give the title compound as a white solid (190 mg, crude). LCMS m/z=606 [M+H]⁺.

### Step 4. Synthesis of methyl 4-amino-1-(2,6-dimethyl-4-((methylsulfonyl)methyl)phenyl)-6-oxo-1,6-dihydropyrimidine-5-carboxylate

A solution of methyl 4-(bis(4-methoxybenzyl)amino)-1-(2,6-dimethyl-4-((methylsulfonyl)methyl)phenyl)-6-oxo-1,6-dihydropyrimidine-5-carboxylate (180 mg, 0.297 mmol) in TFA (2 mL) was stirred at RT. The reaction mixture was concentrated under reduced pressure and the residue was extracted with DCM (3×10 mL). The combined organics were dried (Na₂SO₄) and evaporated to dryness. The residue was purified by prep-TLC (10% EtOAc/MeOH) to give the title compound as a yellow oil (90 mg, 83%). LCMS m/z=366 [M+H]⁺.

### Intermediate B54. methyl(R)-4-amino-1-(4-(1-fluoroethyl)-2,6-dimethylphenyl)-6-oxo-1,6-dihydropyrimidine-5-carboxylate or methyl(S)-4-amino-1-(4-(1-fluoroethyl)-2,6-dimethylphenyl)-6-oxo-1,6-dihydropyrimidine-5-carboxylate

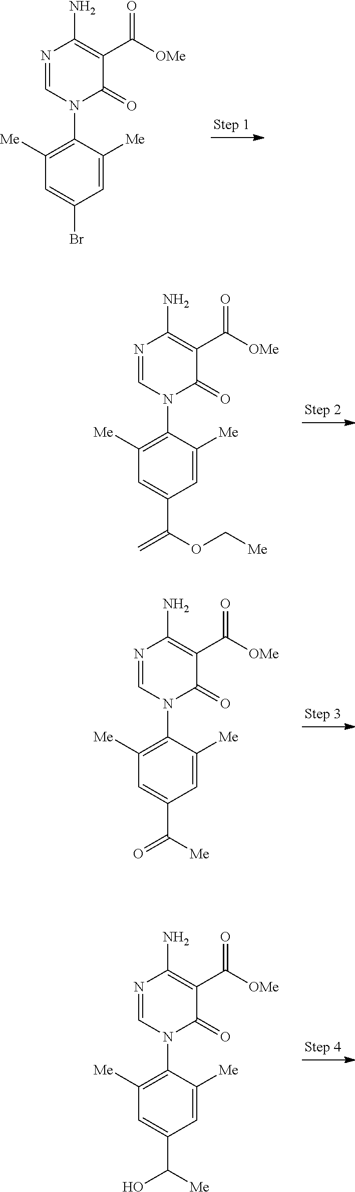 [SMILES: COC(=O)c1c(N)ncn(-c2c(C)cc(Br)cc2C)c1=O ; C=C(OCC)c1cc(C)c(-n2cnc(N)c(C(=O)OC)c2=O)c(C)c1 ; COC(=O)c1c(N)ncn(-c2c(C)cc(C(C)=O)cc2C)c1=O ; COC(=O)c1c(N)ncn(-c2c(C)cc(C(C)O)cc2C)c1=O]

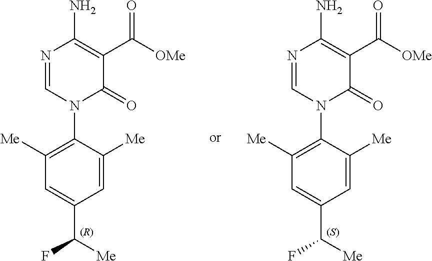

### Step 1. Synthesis of methyl 4-amino-1-(4-(1-ethoxyvinyl)-2,6-dimethylphenyl)-6-oxo-1,6-dihydropyrimidine-5-carboxylate

A mixture of methyl 4-amino-1-(4-bromo-2,6-dimethylphenyl)-6-oxo-1,6-dihydropyrimidine-5-carboxylate (Intermediate B34, 3 g, 8.52 mmol) tributyl(1-ethoxyvinyl) stannane (4.61 g, 12.8 mmol), Pd(PPh₃)₄ (984 mg, 0.85 mmol) in dioxane (80 mL) was degassed and purged with N₂ (×3) and the mixture stirred at 80° C. for 12 h under N₂. The reaction mixture was quenched by addition saturated aqueous KF solution (30 mL) at 0° C. and then extracted with EtOAc (3×20 mL). The combined organics were dried (Na₂SO₄) and concentrated under reduced pressure and the residue purified by column chromatography (SiO₂, 0-50% EtOAc/PE) to give the title compound as a yellow solid (2 g, 68%). LCMS m/z=344 [M+H]⁺.

### Step 2. Synthesis of methyl 1-(4-acetyl-2,6-dimethylphenyl)-4-amino-6-oxo-1,6-dihydropyrimidine-5-carboxylate

A mixture of methyl 4-amino-1-(4-(1-ethoxyvinyl)-2,6-dimethylphenyl)-6-oxo-1,6-dihydropyrimidine-5-carboxylate (Step 1, 2 g, 5.82 mmol), citric acid (2 M, 75 mL) in THF (15 mL) was degassed and the mixture stirred at 25° C. for 40 min. The reaction mixture was concentrated under reduced pressure and the residue diluted with H₂O (10 mL) and extracted with EtOAc (3×10 mL). The combined organics were dried (Na₂SO₄) and concentrated under reduced pressure to give the title compound as a white solid (3.60 g, 98%). LCMS m/z=316 [M+H]⁺.

### Step 3. Synthesis of methyl 4-amino-1-(4-(1-hydroxyethyl)-2,6-dimethylphenyl)-6-oxo-1,6-dihydropyrimidine-5-carboxylate

To a mixture of methyl 1-(4-acetyl-2,6-dimethylphenyl)-4-amino-6-oxo-1,6-dihydropyrimidine-5-carboxylate (Intermediate B54 Step 2, 1.50 g, 4.76 mmol) in MeOH (40 mL) was added NaBH₄ (360 mg, 9.51 mmol) at 0° C., and then the mixture was stirred at 0° C. for 2 min. The reaction was quenched with aq. Na₂CO₃ (10 mL) until pH=10 and the mixture was extracted with EtOAc (4×10 mL). The combined organics were dried (Na₂SO₄) and concentrated under reduced pressure. The residue was purified by prep-HPLC (C18; 250×50 mm, 10 μm); 10-35% MeCN/H₂O(NH₄HCO₃)) to give the title compound as a white solid (1.00 g, 33%). LCMS m/z=318 [M+H]⁺.

### Step 4. Synthesis of methyl(R)-4-amino-1-(4-(1-fluoroethyl)-2,6-dimethylphenyl)-6-oxo-1,6-dihydropyrimidine-5-carboxylate or methyl(S)-4-amino-1-(4-(1-fluoroethyl)-2,6-dimethylphenyl)-6-oxo-1,6-dihydropyrimidine-5-carboxylate

To a solution of methyl 4-amino-1-(4-(1-hydroxyethyl)-2,6-dimethylphenyl)-6-oxo-1,6-dihydropyrimidine-5-carboxylate (Step 3, 410 mg, 1.29 mmol) in DCM (8 mL) was added DAST (479 mg, 2.97 mmol) at −78° C. under N₂ and the mixture stirred at −78° C. for 3 h and 25° C. for 9 h. The reaction mixture was quenched by addition saturated NaHCO₃ (30 mL) at 0° C. and extracted with EtOAc (3×30 mL). The combined organics were dried (Na₂SO₄) and concentrated under reduced pressure and the residue separated by SFC (DAICEL CHIRALCEL OJ, 250×30 mm, 10 μm); 20% IPA (0.1% NH₄OH)) to afford:

Peak 2, Intermediate B54: methyl(R)-4-amino-1-(4-(1-fluoroethyl)-2,6-dimethylphenyl)-6-oxo-1,6-dihydropyrimidine-5-carboxylate or methyl(S)-4-amino-1-(4-(1-fluoroethyl)-2,6-dimethylphenyl)-6-oxo-1,6-dihydropyrimidine-5-carboxylate (white solid, 190 mg, 46%); LCMS m/z=320 [M+H]⁺.

### Intermediate B55. Synthesis of methyl 4-amino-1-(4-(1,1-difluoroethyl)-2,6-dimethylphenyl)-6-oxo-1,6-dihydropyrimidine-5-carboxylate

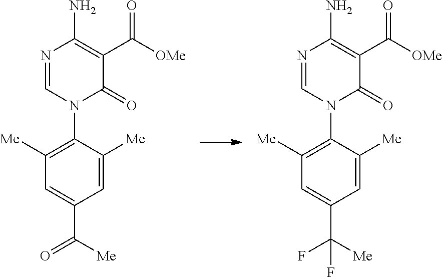 [SMILES: COC(=O)c1c(N)ncn(-c2c(C)cc(C(C)=O)cc2C)c1=O ; COC(=O)c1c(N)ncn(-c2c(C)cc(C(C)(F)F)cc2C)c1=O]

A solution of methyl 1-(4-acetyl-2,6-dimethylphenyl)-4-amino-6-oxo-1,6-dihydropyrimidine-5-carboxylate (Intermediate B54, Step 2; 120 mg, 0.38 mmol) in DAST (4 mL) was stirred at 45° C. for 12 h under N₂. The reaction mixture was quenched by addition of sat aq NH₄Cl (15 mL) at 0° C. and then extracted with EtOAc (3×20 mL). The combined organics were washed with brine (10 mL), dried (Na₂SO₄) and concentrated under reduced pressure. The residue was purified by TLC (100% EtOAc1) to give the title compound as a yellow solid (80 mg, 62%). LCMS m/z=338 [M+H]⁺.

### Intermediate B56. methyl 4-amino-1-(2,6-dimethyl-4-(trifluoromethyl)phenyl)-6-thioxo-1,6-dihydropyrimidine-5-carboxylate

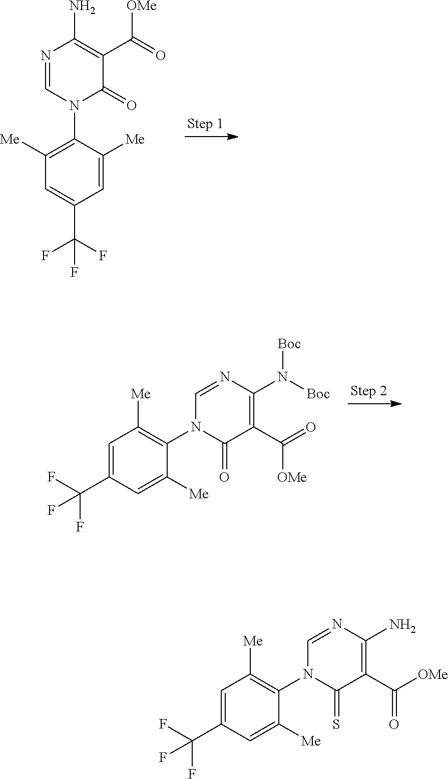 [SMILES: COC(=O)c1c(N)ncn(-c2c(C)cc(C(F)(F)F)cc2C)c1=O ; COC(=O)c1c(N(C(=O)OC(C)(C)C)C(=O)OC(C)(C)C)ncn(-c2c(C)cc(C(F)(F)F)cc2C)c1=O ; COC(=O)c1c(N)ncn(-c2c(C)cc(C(F)(F)F)cc2C)c1=S]

### Step 1. Synthesis of methyl 4-(bis(tert-butoxycarbonyl)amino)-1-(2,6-dimethyl-4-(trifluoromethyl)phenyl)-6-oxo-1,6-dihydropyrimidine-5-carboxylate

To a suspension of methyl 4-amino-1-(2,6-dimethyl-4-(trifluoromethyl)phenyl)-6-oxo-1,6-dihydropyrimidine-5-carboxylate (Intermediate B24, 607 mg, 1.76 mmol) in THF (9 mL) were added DMAP (21.4 mg, 0.176 mmol), triethylamine (0.50 mL, 3.52 mmol), and Boc₂O (1.085 g, 5.28 mmol) sequentially at RT. The reaction mixture was stirred at RT for 5 h. The reaction was diluted with EtOAc (50 mL) and washed with water, saturated aqueous NaHCO₃, brine, dried (MgSO₄) and concentrated by rotary evaporation. Flash chromatography (ISCO 24 g silica, 0-80% EtOAc/Hex) provided the title compound as a yellow solid (755 mg, 79%). LCMS m/z=542 [M+H]⁺.

### Step 2. Synthesis of methyl 4-amino-1-(2,6-dimethyl-4-(trifluoromethyl)phenyl)-6-thioxo-1,6-dihydropyrimidine-5-carboxylate

A suspension of methyl 4-(bis(tert-butoxycarbonyl)amino)-1-(2,6-dimethyl-4-(trifluoromethyl)phenyl)-6-oxo-1,6-dihydropyrimidine-5-carboxylate (Step 1, 412 mg, 0.76 mmol) and Lawesson's reagent (308 mg, 0.76 mmol) in anhydrous toluene (3 mL) was heated to 110° C. for 24 h. The reaction was cooled, diluted with EtOAc (40 mL) and washed with dilute aqueous HCl, water and brine. The organics were dried (MgSO₄) and concentrated by rotary evaporation. The residue was purified by flash chromatography (ISCO 24 g silica, 0-65% EtOAc/hex) to afford a mixture of Boc-protected intermediates as a yellow foam (366 mg). The yellow foam (366 mg) was dissolved in DCM (4 mL) and TFA (1 mL) and stirred at RT for 2 h. The reaction was poured into saturated aqueous NaHCO₃ and extracted with DCM (3×15 mL). The combined organics were washed with water and brine, dried over (MgSO₄) and concentrated by rotary evaporation. Flash chromatography (ISCO 24 g silica, 0-100% EtOAc/hex) provided the title compound as a yellow solid (63.5 mg, 23% yield over 2 steps). LCMS m/z=358 [M+H]⁺.

### Intermediate B57. methyl 4-amino-1-(4-(methoxymethyl)-2,6-dimethylphenyl)-6-thioxo-1,6-dihydropyrimidine-5-carboxylate

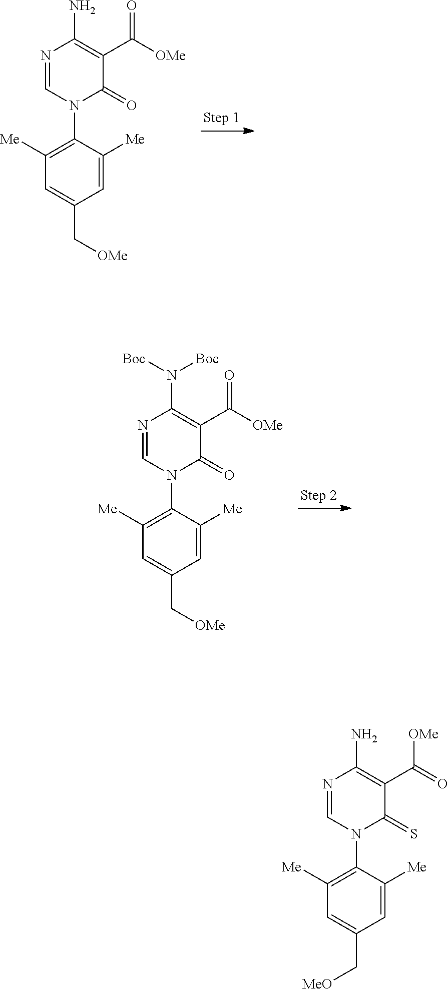 [SMILES: COCc1cc(C)c(-n2cnc(N)c(C(=O)OC)c2=O)c(C)c1 ; COCc1cc(C)c(-n2cnc(N(C(=O)OC(C)(C)C)C(=O)OC(C)(C)C)c(C(=O)OC)c2=O)c(C)c1 ; COCc1cc(C)c(-n2cnc(N)c(C(=O)OC)c2=S)c(C)c1]

### Step 1. Synthesis of methyl 4-(bis(tert-butoxycarbonyl)amino)-1-(4-(methoxymethyl)-2,6-dimethylphenyl)-6-oxo-1,6-dihydropyrimidine-5-carboxylate

To a solution of methyl 4-amino-1-(4-(methoxymethyl)-2,6-dimethylphenyl)-6-oxo-1,6-dihydropyrimidine-5-carboxylate (Intermediate B22, 53.6 mg, 0.170 mmol) in THF (2 mL) were added sequentially at RT, DMAP (3.1 mg, 0.017 mmol), triethylamine (48 μL, 0.340 mmol) and Boc₂O (113 mg, 0.510 mmol) and the reaction was stirred at RT overnight. The reaction mixture was evaporated to dryness and the residue purified by flash chromatography (ISCO, 4 g silica, 0-100% EtOAc/Hex) to provide the title compound as a white solid (77.8 mg, 88%). LCMS m/z=518 [M+H]⁺.

### Step 2. Synthesis of methyl 4-amino-1-(4-(methoxymethyl)-2,6-dimethylphenyl)-6-thioxo-1,6-dihydropyrimidine-5-carboxylate

A suspension of methyl 4-(bis(tert-butoxycarbonyl)amino)-1-(4-(methoxymethyl)-2,6-dimethylphenyl)-6-oxo-1,6-dihydropyrimidine-5-carboxylate (Step 1, 77.8 mg, 0.150 mmol) and Lawesson's reagent (61.7 mg, 0.150 mmol) in anhydrous toluene (0.75 mL) was heated to 110° C. for 19 h. The reaction was cooled, diluted with EtOAc (30 mL), washed with dilute aqueous HCl, water and brine (2×). The combined organics were dried (MgSO₄) and concentrated by rotary evaporation. The residue was purified by flash chromatography (ISCO 4 g silica, 0-75% EtOAc/hex) provided a yellow oil. The yellow oil was dissolved in DCM (2 mL) and TFA (0.5 mL) and stirred at RT for 2 h. The reaction was poured into saturated aqueous NaHCO₃ and extracted with DCM (3×10 mL). The combined organics were washed with brine, dried (MgSO₄) and concentrated by rotary evaporation. Flash chromatography (ISCO 4 g silica, 0-100% EtOAc/hex) to afford the title compound as a yellow solid (13.3 mg, 26% yield over 2 steps). LCMS m/z=334 [M+H]⁺.

### Intermediate B58. Synthesis of methyl 4-amino-1-(4-ethyl-2,6-dimethylphenyl)-6-oxo-1,6-dihydropyrimidine-5-carboxylate

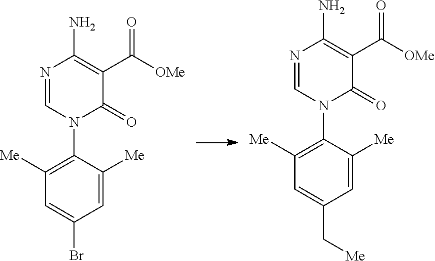 [SMILES: COC(=O)c1c(N)ncn(-c2c(C)cc(Br)cc2C)c1=O ; CCc1cc(C)c(-n2cnc(N)c(C(=O)OC)c2=O)c(C)c1]

The title compound was prepared from methyl 4-amino-1-(4-bromo-2,6-dimethylphenyl)-6-oxo-1,6-dihydropyrimidine-5-carboxylate (Intermediate B34) using an analogous method to that described for Intermediate B35. LCMS m/z=288 [M+H]⁺.

### Intermediate B59. methyl 4-amino-1-(2-methoxyphenyl)-6-oxo-1,6-dihydropyrimidine-5-carboxylate

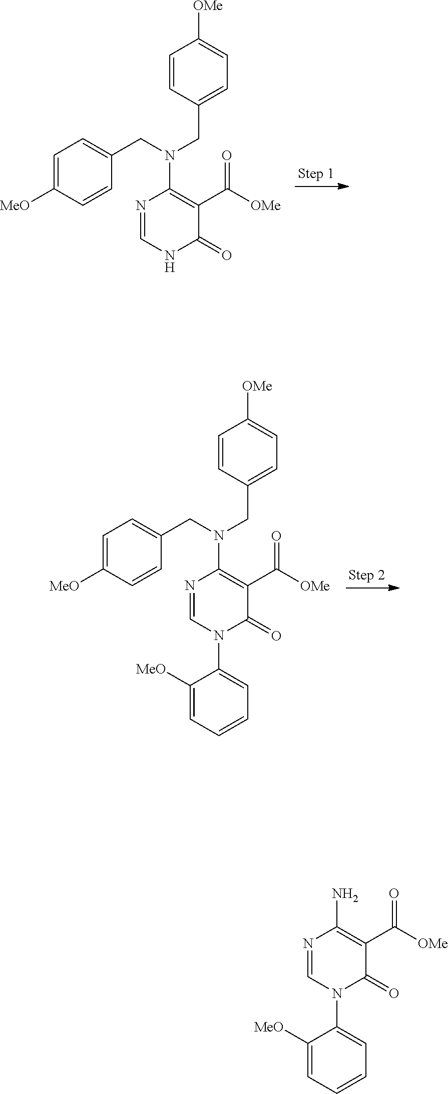 [SMILES: COC(=O)c1c(N(Cc2ccc(OC)cc2)Cc2ccc(OC)cc2)nc[nH]c1=O ; COC(=O)c1c(N(Cc2ccc(OC)cc2)Cc2ccc(OC)cc2)ncn(-c2ccccc2OC)c1=O ; COC(=O)c1c(N)ncn(-c2ccccc2OC)c1=O]

### Step 1. Synthesis of methyl 4-(bis(4-methoxybenzyl)amino)-1-(2-methoxyphenyl)-6-oxo-1,6-dihydropyrimidine-5-carboxylate

To methyl 4-(bis(4-methoxybenzyl)amino)-6-oxo-1,6-dihydropyrimidine-5-carboxylate BP1491664_A1 (Intermediate B1, 400 mg, 0.977 mmol) and (2-methoxyphenyl) boronic acid (445 mg, 2.93 mmol) in DMF (4.00 mL) was added pyridine (232 mg, 2.93 mmol) and Cu(OAc)₂ (266 mg, 1.47 mmol) and the mixture stirred at 100° C. for 12 h under O₂ (15 psi). The mixture was filtered, the filtrate concentrated under reduced pressure and the residue purified by MPLC (SiO₂, 0-50% EtOAc/PE/EtOAc) to give the title compound as a yellow solid (230 mg, crude). LCMS m/z=516 [M+H]⁺.

### Step 2. Synthesis of methyl 4-amino-1-(2-methoxyphenyl)-6-oxo-1,6-dihydropyrimidine-5-carboxylate

Methyl 4-(bis(4-methoxybenzyl)amino)-1-(2-methoxyphenyl)-6-oxo-1,6-dihydropyrimidine-5-carboxylate (Step 1, 230 mg, 0.446 mmol) in TFA (3 mL) was stirred at 80° C. for 1 h. The reaction mixture was concentrated under reduced pressure and the residue partitioned between EtOAc (3 mL) and saturated aqueous Na₂CO₃ solution (5 mL) at 0° C. The aqueous was extracted with EtOAc (3×5 mL). The combined organics were dried (Na₂SO₄) and concentrated under reduced pressure. The residue was purified by prep-TLC (100% EtOAc) to give the title compound (110 mg, 89%) as an off-white solid. LCMS m/z=276 [M+H]⁺.

### Intermediate B60. methyl 4-amino-1-(2-chloro-6-hydroxyphenyl)-6-oxo-1,6-dihydropyrimidine-5-carboxylate

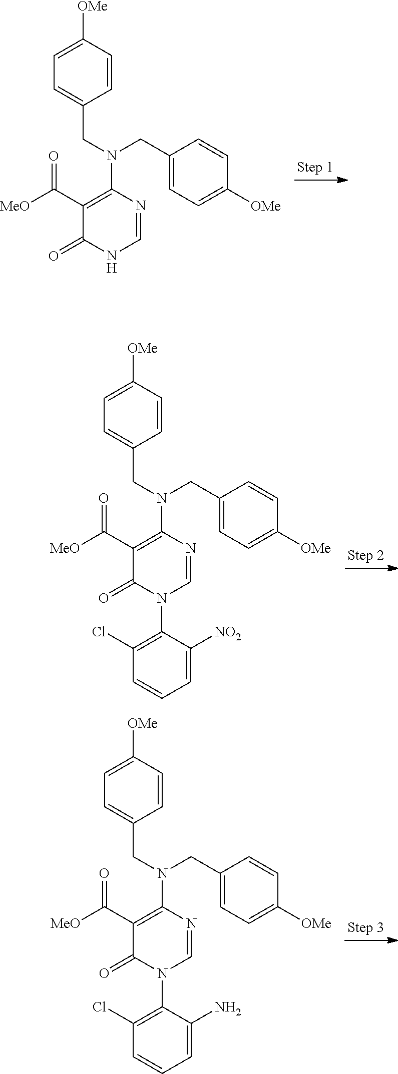

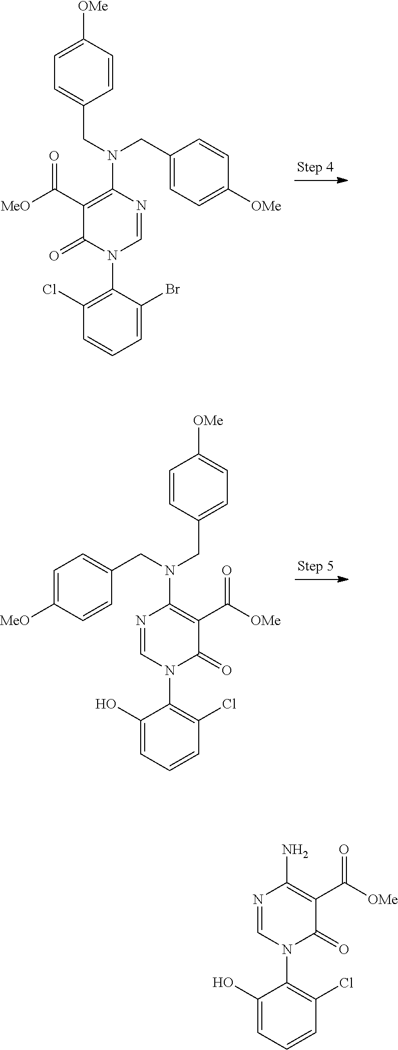 [SMILES: COC(=O)c1c(N(Cc2ccc(OC)cc2)Cc2ccc(OC)cc2)ncn(-c2c(Cl)cccc2Br)c1=O ; COC(=O)c1c(N(Cc2ccc(OC)cc2)Cc2ccc(OC)cc2)ncn(-c2c(O)cccc2Cl)c1=O ; COC(=O)c1c(N)ncn(-c2c(O)cccc2Cl)c1=O]

### Step 1. Synthesis of methyl 4-(bis(4-methoxybenzyl)amino)-1-(2-chloro-6-nitrophenyl)-6-oxo-1,6-dihydropyrimidine-5-carboxylate

The title compound was prepared as a yellow oil (2.3 g, 83%) from methyl 4-(bis(4-methoxybenzyl)amino)-6-oxo-1,6-dihydropyrimidine-5-carboxylate (Intermediate B1) and 1-chloro-2-fluoro-3-nitro-benzene using an analogous method to that described for Intermediate B4, Step 1. LCMS m/z=565 [M+H]⁺.

### Step 2. Synthesis of methyl 1-(2-amino-6-chlorophenyl)-4-(bis(4-methoxybenzyl)amino)-6-oxo-1,6-dihydropyrimidine-5-carboxylate

The title compound was prepared as a yellow oil (2.03 g, 93%) from methyl 4-(bis(4-methoxybenzyl)amino)-1-(2-chloro-6-nitrophenyl)-6-oxo-1,6-dihydropyrimidine-5-carboxylate (Step 1) using an analogous method to that described for Intermediate B3, Step 2. LCMS m/z=535 [M+H]⁺.

### Step 3. Synthesis of methyl 4-(bis(4-methoxybenzyl)amino)-1-(2-bromo-6-chlorophenyl)-6-oxo-1,6-dihydropyrimidine-5-carboxylate

The title compound was prepared as a yellow solid (700 mg, 31%) from methyl 1-(2-amino-6-chlorophenyl)-4-(bis(4-methoxybenzyl)amino)-6-oxo-1,6-dihydropyrimidine-5-carboxylate (Step 2) using an analogous method to that described for Intermediate B4, Step 4. LCMS m/z=600 [M+H]⁺.

### Step 4. Synthesis of methyl 4-(bis(4-methoxybenzyl)amino)-1-(2-chloro-6-hydroxyphenyl)-6-oxo-1,6-dihydropyrimidine-5-carboxylate

The title compound was prepared as a yellow oil (170 mg, 53%) from methyl 4-(bis(4-methoxybenzyl)amino)-1-(2-bromo-6-chlorophenyl)-6-oxo-1,6-dihydropyrimidine-5-carboxylate (Step 3) using an analogous method as described for Intermediate B25, Step 4. LCMS m/z=536 [M+H]⁺.

### Step 5. Synthesis of methyl 4-amino-1-(2-chloro-6-hydroxyphenyl)-6-oxo-1,6-dihydropyrimidine-5-carboxylate

The title compound was prepared as an oil (40m mg, 43%) from methyl 4-(bis(4-methoxybenzyl)amino)-1-(2-chloro-6-hydroxyphenyl)-6-oxo-1,6-dihydropyrimidine-5-carboxylate (Step 4) using an analogous method as described for Intermediate B7, Step 2. LCMS m/z=296 [M+H]⁺.

### Example 1. Synthesis of (S)-4-amino-N-(3-(1-amino-2-methoxyethyl)phenyl)-1-((S)-2-chloro-4-methoxy-6-methylphenyl)-6-oxo-1,6-dihydropyrimidine-5-carboxamide

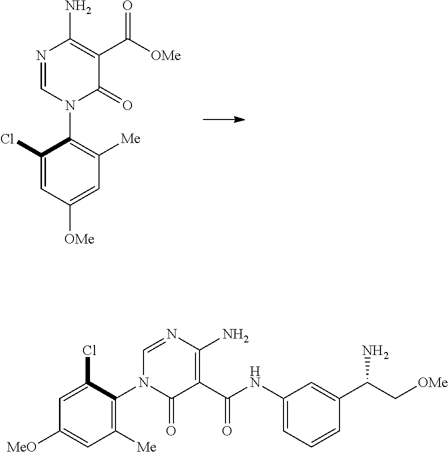

AlMe₃ (2 M in toluene, 69.5 μL) was added to a mixture of methyl 4-amino-1-((S)-2-chloro-4-methoxy-6-methylphenyl)-6-oxo-1,6-dihydropyrimidine-5-carboxylate (Intermediate B4, 15 mg, 0.046 mmol) and (S)-3-(1-amino-2-methoxyethyl) aniline (Intermediate A1, 10.78 mg, 0.065 mmol) in toluene (3 mL) at 0° C. and the mixture stirred at 0° C. for 5 min. The mixture was stirred at 100° C. for 1 h under N₂. The reaction mixture was treated with H₂O (0.2 mL) and TFA (0.2 mL) at 0° C. concentrated under reduced pressure. The residue was purified by prep-HPLC-2 (10-45% MeCN) to give the title compound as a white solid (9.4 mg, 44%). LCMS m/z=458 [M+H]⁺; ¹H NMR (400 MHz, CDCl₃): 11.80 (s, 1H), 10.07 (d, 1H), 7.74 (s, 1H), 7.66 (s, 1H), 7.57 (d, 1H), 7.32-7.29 (m, 1H), 7.13 (d, 1H), 6.99 (d, 1H), 6.85 (d, 1H), 5.95 (d, 1H), 4.20 (dd, 1H), 3.87 (s, 3H), 3.55 (dd, 1H), 3.45 (d, 1H), 3.40 (s, 3H), 2.23 (s, 3H).

### Example 2. Synthesis of (R)-4-amino-1-((S)-2-chloro-4-methoxy-6-methylphenyl)-N-(3-(1-(methylamino)ethyl)phenyl)-6-oxo-1,6-dihydropyrimidine-5-carboxamide

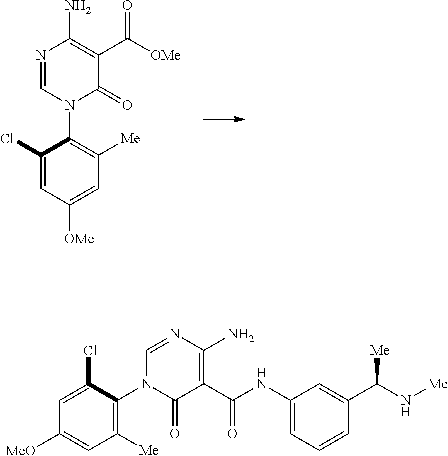

AlMe₃ (2 M in toluene, 46.3 μL) was added to a solution of methyl 4-amino-1-((S)-2-chloro-4-methoxy-6-methyl-phenyl)-6-oxo-pyrimidine-5-carboxylate (Intermediate B4, 10 mg, 0.031 mmol) and (R)-3-(1-(methylamino)ethyl) aniline (Intermediate A2, 6.96 mg, 0.0463 mmol) in toluene (1 mL) and THF (0.5 mL) at 0° C. under N₂ and the mixture was stirred at 40° C. for 1 h under N₂. The reaction mixture was quenched with 10% NaOH solution (5 mL) at 0° C. and extracted with EtOAc (3×3 mL). The combined organics were dried (Na₂SO₄) and evaporated to dryness in vacuo. The residue was purified by prep-HPLC-1 (5-45% MeCN) to give the title compound as a yellow oil (8.1 mg, 53%) as yellow oil. LCMS m/z=442 [M+H]⁺; ¹H NMR (400 MHz, DMSO-d₆): 11.96 (s, 1H), 9.67 (d, 1H), 8.55 (d, 1H), 8.32 (s, 1H), 8.29 (s, 1H), 7.63-7.63 (m, 1H), 7.65 (d, 1H), 7.48 (s, 1H), 7.30 (t, 1H), 7.09-7.04 (m, 2H), 3.86 (s, 3H), 3.74-3.65 (m, 1H), 2.18 (s, 3H), 2.15 (s, 3H), 2.10 (s, 1H), 1.29 (d, 3H).

### Example 3-32

The title compounds were prepared using an analogous method to that described for Example 1

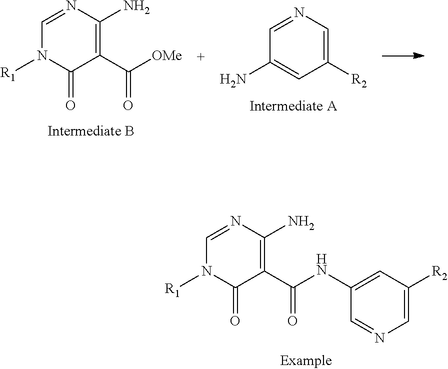

| Example | |
|---|---|
| No | Name, Reactants, HPLC, Data |
| 3 | (R)-4-amino-1-((S)-2-chloro-6-methylphenyl)-N-(3-(1- |
| | (methylamino)ethyl)phenyl)-6-oxo-1,6-dihydropyrimidine-5-carboxamide |
| | Intermediate B3, Intermediate A2 |
| | HPLC-1 (10-44% MeCN); Pale yellow solid (4.4 mg, 10%); LCMS m/z = 413 |
| | [M+H]⁺; ¹H NMR (400 MHZ, DMSO-d6): 11.91 (s, 1H), 9.68 (d, 1H), 8.58 (d, |
| | 1H), 8.33-8.31 (m, 2H), 7.65 (d, 1H), 7.58-7.56 (m, 1H), 7.52-7.50 (m, 1H), |
| | 7.47-7.45 (m, 2H), 7.29 (t, 1H), 7.06 (d, 1H), 3.70 (d, 1H), 2.19 (d, 6H, J), 1.29 |
| | (d, 3H). |
| 4 | (R)-4-amino-1-((S)-2-chloro-4-methoxy-6-methylphenyl)-N-(5-(1-((methyl- |
| | d3)amino)ethyl)pyridin-3-yl)-6-oxo-1,6-dihydropyrimidine-5-carboxamide or |
| | (S)-4-amino-1-((S)-2-chloro-4-methoxy-6-methylphenyl)-N-(5-(1-((methyl- |
| | d3)amino)ethyl)pyridin-3-yl)-6-oxo-1,6-dihydropyrimidine-5-carboxamide |
| | Intermediate B4, Intermediate A54 |
| | HPLC-1 (1-40% MeCN); White solid (29.9 mg, 49%); LCMS m/z = 446 |
| | [M+H]⁺; ¹H NMR (400 MHZ, CDC13): 11.98 (s, 1H), 9.98 (br d, 1H), 8.68 (d, |
| | 1H), 8.31-8.37 (m, 2H), 7.76 (s, 1H), 6.99 (d, 1H), 6.85 (d, 1H), 6.06 (br d, 1H), |
| | 3.94 (q, 1H), 3.87 (s, 3H), 2.23 (s, 3H), 1.58 (d, 3H). |
| 5 | 4-amino-1-(2,6-dichlorophenyl)-N-(5-((ethylamino)methyl)pyridin-3-yl)-6-oxo- |
| | 1,6-dihydropyrimidine-5-carboxamide |
| | Intermediate B9, Intermediate A12 |
| | HPLC-1 (3-35% MeCN); White solid (2.5 mg, 9%); LCMS m/z = 433 [M+H]⁺; |
| | 1H NMR (400 MHZ, CDC13): 11.78 (s, 1H), 10.00 (s, 1H), 8.61 (d, 1H), 8.44 (s, |
| | 1H), 8.38 (s, 1H), 7.74-7.72 (m, 1H), 7.56-7.54 (m, 2H), 7.47-7.43 (m, 1H), 6.06 |
| | (s, 1H), 4.00 (s, 2H), 2.87 (q, 2H), 1.26 (t, 3H). |
| 6 | (R)-4-amino-1-(2,6-dichlorophenyl)-N-(3-fluoro-5-(1- |
| | (methylamino)ethyl)phenyl)-6-oxo-1,6-dihydropyrimidine-5-carboxamide |
| | trifluoroacetate |
| | Intermediate B9, Intermediate A67 |
| | HPLC-10 (20-50% MeCN); White solid (19.4 mg, 23%); LCMS m/z = 450 |
| | [M+H]⁺; 1H NMR (500 MHZ, DMSO-d6): 12.02 (s, 1H), 9.53 (d, 1H), 8.86 (s, |
| | ¹H), 8.74 (d, 1H), 8.66 (s, 1H), 8.41 (s, 1H), 7.82 (dt, 1H), 7.71-7.68 (m, 2H), |
| | 7.57 (t, 1H), 7.25 (s, 1H), 6.97 (d, 1H), 4.20 (q, 1H), 2.36 (t, 3H), 1.43 (d, 3H). |
| 7 | (S)-4-amino-N-(3-(1-amino-2-methoxyethyl)phenyl)-1-((S)-2-chloro-6- |
| | (difluoromethyl)phenyl)-6-oxo-1,6-dihydropyrimidine-5-carboxamide |
| | Intermediate B48, Intermediate A1 |
| | HPLC-1 (1-45% MeCN); White solid (3.4 mg, 14%); LCMS m/z = 464 [M+H]⁺; |
| | ¹H NMR (400 MHZ, CDC13): 11.57 (s, 1H), 10.12 (d, 1H), 7.84-7.69 (m, 3H), |
| | 7.69-7.57 (m, 2H), 7.53 (d, 1H), 7.34-7.29 (m, 1H), 7.14 (d, 1H), 6.86-6.45 (m, |
| | 1H), 6.10 (d, 1H), 4.22 (dd, 1H), 3.60-3.54 (m, 1H), 3.53-3.45 (m, 1H), 3.39 (s, |
| | 3H). |
| 8 | (R)-4-amino-N-(3-(1-aminoethyl)phenyl)-1-((S)-2-chloro-4-cyano-6- |
| | methylphenyl)-6-oxo-1,6-dihydropyrimidine-5-carboxamide |
| | Intermediate B13, Intermediate A: (R)-3-(1-aminoethyl)aniline |
| | HPLC-2 (8-40%); Pale yellow solid (3.8 mg, 9%); LCMS m/z = 406 [M+H]⁺; |
| | ¹H NMR (400 MHZ, DMSO-d6): 11.78 (s, 1H), 9.71 (d, 1H), 8.68 (d, 1H), 8.38- |
| | 8.34 (m, 1H), 8.25 (d, 1H), 8.03 (d, 1H), 7.69-7.65 (m, 1H), 7.50 (s, 1H), 7.29 (t, |
| | 1H), 7.11 (d, 1H), 4.11 (q, 1H), 2.24 (s, 3H), 1.33 (d, 3H). |
| 9 | (R)-4-amino-1-(2,6-dichloro-4-cyanophenyl)-N-(5-(1- |
| | (methylamino)ethyl)pyridin-3-yl)-6-oxo-1,6-dihydropyrimidine-5-carboxamide |
| | or (S)-4-amino-1-(2,6-dichloro-4-cyanophenyl)-N-(5-(1- |
| | (methylamino)ethyl)pyridin-3-yl)-6-oxo-1,6-dihydropyrimidine-5-carboxamide |
| | Intermediate B14, Intermediate A14 |
| | HPLC-2 (20-60% MeCN); Off-white solid (19.4 mg, 58%); LCMS m/z = 458 |
| | [M+H]⁺; ¹H NMR (400 MHZ, CDC13): 1.75 (s, 3H) 2.55 (s, 3H) 4.18-4.25 (m, |
| | 1H) 6.23-6.28 (m, 1H) 7.73 (s, 1H) 7.88 (s, 2H) 8.37 (s, 1H) 8.57 (s, 1H) 8.64 |
| | (d, 1H) 10.07 (d, 1H) 11.70 (s, 1H). |
| 10 | 4-amino-1-(2,6-dichloro-4-(difluoromethoxy)phenyl)-N-(5- |
| | ((ethylamino)methyl)pyridin-3-yl)-6-oxo-1,6-dihydropyrimidine-5-carboxamide |
| | Intermediate B45, Intermediate A12 |
| | HPLC-2 (3-33% MeCN); Colourless oil (6.8 mg, 20%); LCMS m/z = 499 |
| | [M+H]⁺; ¹H NMR (400 MHZ, DMSO-d6): 11.76 (s, 1H), 9.65 (s, 1H), 8.79 (d, |
| | 1H), 8.68 (s, 1H), 8.45 (s, 1H), 8.23 (s, 1H), 8.04 (s, 1H), 7.73 (s, 2H), 7.50 (t, |
| | 1H), 3.75 (s, 2H), 2.58-2.56 (m, 2H), 1.04 (t, 3H). |
| 11 | 4-amino-1-((S)-2-chloro-4-(difluoromethyl)-6-methylphenyl)-N-(3- |
| | ((ethylamino)methyl)phenyl)-6-oxo-1,6-dihydropyrimidine-5-carboxamide |
| | Intermediate B46, Intermediate A41 |
| | HPLC-1 (20-50% MeCN); White solid (20.9 mg, 47%); LCMS m/z = 462 |
| | [M+H]⁺; ¹H NMR (400 MHZ, CDC13): 11.66 (s, 1H), 10.05 (br d, 1H), 7.83 (s, |
| | 1H), 7.70 (s, 1H), 7.61 (s, 1H), 7.46 (s, 1H), 7.39 (br d, 1H), 7.30-7.27 (m, 1H), |
| | 7.12 (br d, 1H), 6.80-6.51 (m, 1H), 6.07 (br d, 1H), 3.91 (s, 2H), 2.84 (q, 2H), |
| | 2.31 (s, 3H), 1.19 (t, 3H). |
| 12 | (R)-4-amino-N-(5-(1-(methylamino)ethyl)pyridin-3-yl)-6-oxo-1-(2,4,6- |
| | trichlorophenyl)-1,6-dihydropyrimidine-5-carboxamide or (S)-4-amino-N-(5-(1- |
| | (methylamino)ethyl)pyridin-3-yl)-6-oxo-1-(2,4,6-trichlorophenyl)-1,6- |
| | dihydropyrimidine-5-carboxamide |
| | Intermediate B15, Intermediate A14 |
| | HPLC-2 (5-35% MeCN); White solid (12 mg, 28%); LCMS m/z = 468 [M+H]⁺; |
| | ¹H NMR (400 MHZ, CDC13): 11.63 (s, 1H), 9.99-9.98 (m, 1H), 8.60 (br d, 1H), |
| | 8.20 (s, 1H), 8.12 (br s, 1H), 7.63 (s, 1H), 7.50 (s, 2H), 6.13 (br s, 1H), 3.73 (br |
| | d, 1H), 2.27 (s, 3H), 1.39 (br d, 3H). |
| 13 | (S)-4-amino-N-(5-(1-(methylamino)ethyl)pyridin-3-yl)-6-oxo-1-(2,4,6- |
| | trichlorophenyl)-1,6-dihydropyrimidine-5-carboxamide or (R)-4-amino-N-(5-(1- |
| | (methylamino)ethyl)pyridin-3-yl)-6-oxo-1-(2,4,6-trichlorophenyl)-1,6- |
| | dihydropyrimidine-5-carboxamide |
| | Intermediate B15, Intermediate A15 |
| | HPLC-1 (10-30); White solid (9.3 mg, 15%); LCMS m/z = 468 [M+H]⁺; ¹H |
| | NMR (400 MHZ, CDCl3): 1.43-1.47 (m, 1H) 1.51 (d, 3H) 2.21 (d, 1H) 2.28 (s, |
| | 1H) 2.35-2.43 (m, 3H) 2.44-2.48 (m, 1H) 2.78-3.23 (m, 6H) 3.82-3.91 (m, 1H) |
| | 6.16-6.39 (m, 1H) 7.13-7.17 (m, 1H) 7.25-7.28 (m, 1H) 7.34-7.41 (m, 1H) 7.47- |
| | 7.53 (m, 1H) 7.58-7.63 (m, 1H) 7.71-7.78 (m, 1H) 8.24-8.36 (m, 2H) 8.67-8.72 |
| | (m, 1H) 9.98-10.14 (m, 1H) 11.74 (s, 1H) 11.85 (d, 1H) 11.96-12.01 (m, 1H). |
| 14 | 4-amino-1-(2,6-dichloro-4-(difluoromethyl)phenyl)-N-(5- |
| | ((ethylamino)methyl)pyridin-3-yl)-6-oxo-1,6-dihydropyrimidine-5-carboxamide |
| | Intermediate B16, Intermediate A12 |
| | HPLC-2 (3-33% MeCN); Orange solid (8.1 mg, 24%); LCMS m/z = 483 |
| | [M+H]⁺; ¹H NMR (400 MHZ, DMSO-d6): 11.73 (s, 1H), 9.67 (s, 1H), 8.83 (d, |
| | 1H), 8.68 (s, 1H), 8.49 (s, 1H), 8.23 (s, 1H), 8.04 (s, 3H), 7.16 (t, 1H), 3.75 (s, |
| | 2H), 2.58-2.56 (m, 2H), 1.04 (t, 3H). |
| 15 | 4-amino-N-(5-(2-aminopropan-2-yl)pyridin-3-yl)-1-(2,6-dichloro-4- |
| | (methoxymethyl)phenyl)-6-oxo-1,6-dihydropyrimidine-5-carboxamide |
| | Intermediate B19, Intermediate A52 |
| | HPLC-1 (1-40% MeCN); Yellow solid (25.2 mg, 90%); LCMS m/z = 476 |
| | [M+H]⁺; ¹H NMR (400 MHZ, CDC13): 11.78 (s, 1H), 9.95 (s, 1H), 8.62 (s, 1H), |
| | 8.49 (s, 1H), 8.34 (s, 1H), 7.73 (s, 1H), 7.52 (s, 2H), 6.57 (d, 1H), 4.51 (s, 2H), |
| | 3.48 (s, 3H), 1.66 (s, 6H). |
| 16 | (R)-4-amino-1-((S)-2-chloro-6-methyl-4-(trifluoromethyl)phenyl)-N-(5-(1- |
| | ((methyl-d3)amino)ethyl)pyridin-3-yl)-6-oxo-1,6-dihydropyrimidine-5- |
| | carboxamide or (S)-4-amino-1-((S)-2-chloro-6-methyl-4- |
| | (trifluoromethyl)phenyl)-N-(5-(1-((methyl-d3)amino)ethyl)pyridin-3-yl)-6-oxo- |
| | 1,6-dihydropyrimidine-5-carboxamide |
| | Intermediate B47, Intermediate A54 |
| | HPLC-1 (1-40% MeCN); White solid (34.6 mg, 64%); LCMS m/z = 484 |
| | [M+H]⁺; ¹H NMR (400 MHZ, CDC13): 11.84 (s, 1H), 10.06 (d, 1H), 8.66 (s, 1H), |
| | 8.44 (s, 1H), 8.37 (s, 1H), 7.76 (d, 2H), 7.63 (s, 1H), 6.31 (d, 1H), 4.03 (q, 1H), |
| | 2.37 (s, 3H), 1.64 (d, 3H). |
| 17 | 4-amino-1-(2,6-dichloro-4-methoxyphenyl)-N-(5-((ethylamino)methyl)pyridin- |
| | 3-yl)-6-oxo-1,6-dihydropyrimidine-5-carboxamide |
| | Intermediate B10, Intermediate A12 |
| | HPLC-2 (3-35% MeCN); White solid 2.1 mg, 5%); LCMS m/z = 463 [M+H]⁺; |
| | ¹H NMR (400 MHZ, CDC13): 11.74 (s, 1H), 9.92 (d, 1H), 8.58 (d), 8.31 (s, 1H), |
| | 8.26 (s, 1H), 7.66-7.65 (m, 1H), 6.99-6.98 (m, 2H), 5.94 (s, 1H), 3.87 (s, 2H), |
| | 3.80 (s, 3H), 2.72 (q, 2H), 1.18 (t, 3H). |
| 18 | (S)-4-amino-1-(2,6-dichloro-4-methoxyphenyl)-N-(5-(1- |
| | (methylamino)ethyl)pyridin-3-yl)-6-oxo-1,6-dihydropyrimidine-5-carboxamide |
| | or (R)-4-amino-1-(2,6-dichloro-4-methoxyphenyl)-N-(5-(1- |
| | (methylamino)ethyl)pyridin-3-yl)-6-oxo-1,6-dihydropyrimidine-5-carboxamide |
| | Intermediate B10, Intermediate A14 |
| | HPLC-11 (20-50% MeCN); White solid (8.8 mg, 31%); LCMS m/z = 463 |
| | [M+H]⁺; ¹H NMR (400 MHZ, CDC13): 11.79 (s, 1H), 10.06 (d, 1H), 8.69 (d, |
| | 1H), 8.25 (d, 1H), 8.11 (t, 1H), 7.74 (s, 1H), 7.07 (s, 2H), 6.22 (d, 1H), 3.88 (s, |
| | 3H), 3.69 (q, 1H), 2.31 (s, 3H), 1.38 (d, 3H). |
| 19 | (R)-4-amino-1-(2,6-dichloro-4-methoxyphenyl)-N-(5-(1- |
| | (methylamino)ethyl)pyridin-3-yl)-6-oxo-1,6-dihydropyrimidine-5-carboxamide |
| | or (S)-4-amino-1-(2,6-dichloro-4-methoxyphenyl)-N-(5-(1- |
| | (methylamino)ethyl)pyridin-3-yl)-6-oxo-1,6-dihydropyrimidine-5-carboxamide |
| | Intermediate B10, Intermediate A15 |
| | HPLC-2 (5-40% MeCN); White solid (21.2 mg, 56%); LCMS m/z = 463 |
| | [M+H]⁺; ¹H NMR (400 MHZ, CDC13): 11.90 (s, 1H), 10.00 (d, 1H), 8.67 (d, |
| | 1H), 8.49 (s, 1H), 8.38 (d, 1H), 8.33 (d, 1H), 7.75 (s, 1H), 7.09 (s, 2H), 6.15 (d, |
| | 1H), 4.00 (q, 1H), 3.90 (s, 3H), 2.41 (s, 3H), 1.61 (d, 3H). |
| 20 | 4-amino-1-(2,6-dichloro-4-methoxyphenyl)-N-(5- |
| | ((isopropylamino)methyl)pyridin-3-yl)-6-oxo-1,6-dihydropyrimidine-5- |
| | carboxamide |
| | Intermediate B10, Intermediate A25 |
| | HPLC-2 (5-35% MeCN); White solid (38.8 mg, 50%); LCMS m/z = 477 |
| | [M+H]⁺; ¹H NMR (400 MHZ, CDC13): 11.83 (s, 1H), 10.00 (br d, 1H), 8.63 (d, |
| | 1H), 8.34 (s, 2H), 7.74 (s, 1H), 7.08 (s, 2H), 6.11 (br d, 1H), 3.93 (s, 2H), 3.90 |
| | (s, 3H), 3.02 (td, 1H), 1.22 (d, 6H). |
| 21 | 4-amino-N-(5-(1-aminocyclopentyl)pyridin-3-yl)-1-(2,6-dichloro-4- |
| | methoxyphenyl)-6-oxo-1,6-dihydropyrimidine-5-carboxamide |
| | Intermediate B10, Intermediate A39 |
| | HPLC-3 (15-40% MeCN); Yellow solid (7.2 mg, 18%); LCMS m/z = 489 |
| | [M+H]⁺; ¹H NMR (400 MHZ, CDC13): 11.78 (s, 1H), 10.08 (d, 1H), 8.79-8.71 |
| | (m, 1H), 8.48 (s, 1H), 8.19 (s, 1H), 7.76 (s, 1H), 7.10 (s, 2H), 6.08 (d, 1H), 3.91 |
| | (s, 3H), 2.09-1.91 (m, 8H). |
| 22 | 4-amino-1-(2,6-dichloro-4-(trifluoromethyl)phenyl)-N-(5- |
| | ((ethylamino)methyl)pyridin-3-yl)-6-oxo-1,6-dihydropyrimidine-5-carboxamide |
| | Intermediate B23, Intermediate A12 |
| | HPLC-1 (5-45% MeCN); White solid (75 mg, 80%); LCMS m/z = 503 [M+H]⁺; |
| | ¹H NMR (400 MHZ, CDCl3): 11.67 (s, 1H), 10.09 (s, 1H), 8.61 (s, 1H), 8.45- |
| | 8.34 (m, 3H), 7.85 (s, 2H), 7.74 (s, 1H), 6.21 (d, 1H), 3.97 (s, 2H), 2.84 (q, 2H), |
| | 1.22 (t, 3H). |
| 23 | 4-amino-1-(2,6-dichloro-4-(trifluoromethyl)phenyl)-N-(5-(((2- |
| | hydroxyethyl)amino)methyl)pyridin-3-yl)-6-oxo-1,6-dihydropyrimidine-5- |
| | carboxamide |
| | Intermediate B23, Intermediate A71 |
| | HPLC-1 (1-40% MeCN); White solid (35.3 mg, 39%); LCMS m/z = 518 |
| | [M+H]⁺; ¹H NMR (400 MHZ, CDC13): 11.69 (s, 1H) 10.02 (br d, 1H) 8.57 (br s, |
| | 1H) 8.44 (br s, 1H) 8.35 (br s, 1H) 7.83 (s, 2H) 7.72 (s, 1H) 6.38 (br d, 1H) 4.07 |
| | (br s, 2H) 3.79 (br s, 2H) 2.99 (br s, 2H). |
| 24 | (R)-4-amino-1-(2,6-dichloro-4-ethoxyphenyl)-N-(5-(1- |
| | (methylamino)ethyl)pyridin-3-yl)-6-oxo-1,6-dihydropyrimidine-5-carboxamide |
| | or (S)-4-amino-1-(2,6-dichloro-4-ethoxyphenyl)-N-(5-(1- |
| | (methylamino)ethyl)pyridin-3-yl)-6-oxo-1,6-dihydropyrimidine-5-carboxamide |
| | Intermediate B30, Intermediate A15 |
| | HPLC-1 (5-40% MeCN); White solid (22.7 mg, 38%) |
| | LCMS m/z = 477 [M+H]⁺; ¹H NMR (400 MHZ, CDC13): 11.84 (s, 1H), 10.02 (d, |
| | 1H,), 8.67 (d, 1H), 8.27 (dd, 2H), 7.73 (s, 1H), 7.05 (s, 2H), 6.18 (d, 1H), 4.09 |
| | (q, 2H), 3.85 (q, 1H), 2.36 (s, 3H), 1.51-1.43 (m, 6H). |
| 25 | 4-amino-1-(2,6-dichloro-4-ethoxyphenyl)-N-(5-(((2- |
| | methoxyethyl)amino)methyl)pyridin-3-yl)-6-oxo-1,6-dihydropyrimidine-5- |
| | carboxamide |
| | Intermediate B30, Intermediate A73 |
| | HPLC-3 (5-45% MeCN); White solid (13.1 mg, 31%); LCMS m/z = 507 |
| | [M+H]⁺; ¹H NMR (400 MHZ, CDC13): 1.48 (t, 3H), 2.82 (t, 2H), 3.37 (s, 3H), |
| | 3.53 (t, 2H), 3.85 (s, 2H), 4.10 (q, 2H), 6.02 (s, 1H), 7.06 (s, 2H), 7.74 (s, 1H), |
| | 8.16 (s, 1H), 8.30 (d, 1H), 8.69 (d, 1H), 10.05 (d, 1H), 11.79 (s, 1H). |
| 26 | 4-amino-1-(2,6-dichloro-4-ethoxyphenyl)-N-(5-(((2- |
| | hydroxyethyl)amino)methyl)pyridin-3-yl)-6-oxo-1,6-dihydropyrimidine-5- |
| | carboxamide |
| | Intermediate B30, Intermediate A71 |
| | HPLC-1 (10-40% MeCN); White solid (28.3 mg, 46%); LCMS m/z = 493 |
| | [M+H]⁺; ¹H NMR (400 MHZ, CDC13): 11.87 (s, 1H), 9.93 (d, 1H), 8.59 (d, 1H), |
| | 8.42 (s, 1H), 8.32 (s, 1H), 7.74 (s, 1H), 7.05 (s, 2H), 6.21 (d, 1H) 4.00 - 4.17 (m, |
| | 4H) 3.78 (s, 2H) 2.99 (s, 2H) 1.47 (t, 3H). |
| 27 | (R)-4-amino-N-(7-amino-7-methyl-6,7-dihydro-5H-cyclopenta[c]pyridin-4-yl)- |
| | 1-(4-(methoxymethyl)-2,6-dimethylphenyl)-6-oxo-1,6-dihydropyrimidine-5- |
| | carboxamide or (S)-4-amino-N-(7-amino-7-methyl-6,7-dihydro-5H- |
| | cyclopenta[c]pyridin-4-yl)-1-(4-(methoxymethyl)-2,6-dimethylphenyl)-6-oxo- |
| | 1,6-dihydropyrimidine-5-carboxamide |
| | Intermediate B22, Intermediate A43 |
| | HPLC-1 (1-35% MeCN); White solid (10.8 mg, 38%); LCMS m/z = 449 |
| | [M+H]⁺; ¹H NMR (400 MHZ, CDC13): 11.78 (s, 1H), 9.93 (br d, 1H), 9.26 (s, |
| | 1H), 8.39 (s, 1H), 7.72 (s, 1H), 7.21 (s, 2H), 6.02 (br d, 1H), 4.47 (s, 2H), 3.47 |
| | (s, 3H), 3.10-2.97 (m, 1H), 2.93-2.78 (m, 1H), 2.19 (d, 8H), 1.55 (s, 3H). |
| 28 | 5-(4-amino-1-(2,6-dichloro-4-ethoxyphenyl)-6-oxo-1,6-dihydropyrimidine-5- |
| | carboxamido)pyridazine-3-carboxamide |
| | Intermediate B30, Intermediate A59 |
| | HPLC-1 (20-50% MeCN); Yellow solid (2.9 mg, 4.8%); LCMS m/z = 464 |
| | [M+H]⁺; ¹H NMR (400 MHZ, CDC13): 12.26 (s, 1H), 9.90 (d, 1H), 9.43 (d, 1H), |
| | 8.73 (d, 1H), 7.99 (s, 1H), 7.77 (s, 1H), 7.06 (s, 2H), 6.21-6.13 (m, 1H), 5.71 (d, |
| | 1H), 4.10 (q, 2H), 1.47 (t, 3H). |
| 29 | 5-(4-amino-1-(4-ethoxy-2,6-dimethylphenyl)-6-oxo-1,6-dihydropyrimidine-5- |
| | carboxamido)pyridazine-3-carboxamide |
| | Intermediate B25, Intermediate A59 |
| | HPLC-3 (15-45% MeCN); White solid (5.4 mg, 9%); LCMS m/z = 424 [M+H]⁺; |
| | ¹H NMR (400 MHZ, DMSO-d6): 1.32-1.36 (m, 3H), 2.04 (s, 6H), 4.06 (q, 2H), |
| | 6.81 (s, 2H), 7.87 (s, 1H), 8.24 (s, 1H), 8.49 (s, 1H), 8.66-8.76 (m, 2H), 9.31 (d, |
| | 1H), 9.39 (d, 1H), 12.51 (s, 1H). |
| 30 | 5-(4-amino-1-(4-(methoxymethyl)-2,6-dimethylphenyl)-6-oxo-1,6- |
| | dihydropyrimidine-5-carboxamido)pyridazine-3-carboxamide |
| | Intermediate B22, Intermediate A59 |
| | HPLC-3 (20-45% MeCN); White solid (10.4 mg, 23%); LCMS m/z = 424 |
| | [M+H]⁺; ¹H NMR (400 MHZ, DMSO-d6): 2.09 (s, 6H), 3.33 (s, 3H), 4.42 (s, |
| | 2H), 7.20 (s, 2H), 7.87 (s, 1H), 8.27 (s, 1H), 8.49 (s, 1H), 8.72 (d, 1H), 8.74 (d, |
| | 1H), 9.31 (d, 1H), 9.41 (d, 1H), 12.47 (s, 1H). |
| 31 | (R)-4-amino-N-(5-(2-amino-1-methoxypropan-2-yl)pyridin-3-yl)-1-(2,6- |
| | dichloro-4-methoxyphenyl)-6-oxo-1,6-dihydropyrimidine-5-carboxamide or (S)- |
| | 4-amino-N-(5-(2-amino-1-methoxypropan-2-yl)pyridin-3-yl)-1-(2,6-dichloro-4- |
| | methoxyphenyl)-6-oxo-1,6-dihydropyrimidine-5-carboxamide |
| | Intermediate B10, Intermediate A26 |
| | HPLC-1 (1-40% MeCN); White solid (51.9 mg, 64%); LCMS m/z = 493 |
| | [M+H]⁺; ¹H NMR (400 MHZ, CDC13): 11.83 (s, 1H), 9.93 (d, 1H), 8.70 (d, 1H), |
| | 8.47-8.43 (m, 1H), 8.30 (s, 1H), 7.74-7.71 (m, 1H), 7.06 (s, 2H), 6.24 (d, 1H), |
| | 3.87 (s, 3H), 3.61 (s, 1H), 3.55-3.52 (m, 1H), 3.35 (s, 3H), 1.59 (s, 3H). |
| 32 | 4-amino-1-(2,6-dichloro-4-methoxyphenyl)-N-(5-((1R,3aS,6aR)- |
| | octahydrocyclopenta[c]pyrrol-1-yl)pyridin-3-yl)-6-oxo-1,6-dihydropyrimidine- |
| | 5-carboxamide or 4-amino-1-(2,6-dichloro-4-methoxyphenyl)-N-(5- |
| | ((1S,3aR,6aS)-octahydrocyclopenta[c]pyrrol-1-yl)pyridin-3-yl)-6-oxo-1,6- |
| | dihydropyrimidine-5-carboxamide |
| | Intermediate B10, Intermediate A47 |
| | HPLC-1 (1-50% MeCN); White solid (33.3 mg, 71%); LCMS m/z = 515 |
| | [M+H]⁺; ¹H NMR (400 MHz, DMSO-d6): 11.87 (s, 1H), 9.63 (d, 1H), 8.75-8.69 |
| | (m, 2H), 8.41 (s, 1H), 8.30 (d, 1H), 8.22 (s, 1H), 8.12 (s, 1H), 7.38 (s, 2H), 3.90 |
| | (s, 3H), 3.62 (d, 2H), 3.37-3.31 (m, 1H), 2.77-2.64 (m, 2H), 2.39 (q, 1H), 1.69- |
| | 1.48 (m, 6H). |

### Example 33. Synthesis of 4-amino-1-((S)-2-chloro-6-methyl-4-(trifluoromethyl)phenyl)-N-(3-((R)-1-(methylamino)ethyl)phenyl)-6-oxo-1,6-dihydropyrimidine-5-carboxamide

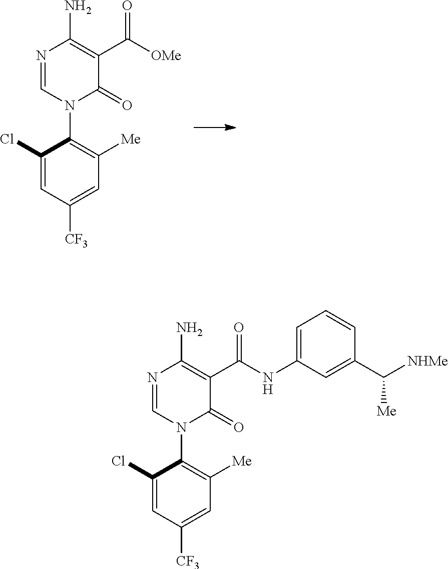

AlMe₃ (2 M in toluene, 104 μL) was added to a solution of methyl 4-amino-1-((S)-2-chloro-6-methyl-4-(trifluoromethyl)phenyl)-6-oxo-1,6-dihydropyrimidine-5-carboxylate (Intermediate B47, 25 mg, 0.069 mmol) and (R)-3-(1-(methylamino)ethyl) aniline (Intermediate A2, 20.77 mg, 0.138 mmol) in toluene (2 mL) at 0° C. and the mixture stirred at 40° C. for 0.5 h under N₂. The reaction mixture was quenched by addition saturated 0.2 M NaOH (5 mL) at 0° C. and extracted with EtOAc (3×5 mL). The combined organics were dried (Na₂SO₄) and evaporated to dryness in vacuo. The residue was purified by prep-HPLC-1 (10-50% MeCN) to give the title compound as a white solid (3.2 mg, 9.7%). LCMS m/z=480 [M+H]⁺; ¹H NMR (400 MHz, CDCl₃): 11.59 (s, 1H), 10.06 (d, 1H), 8.55 (s, 1H), 7.65-7.62 (m, 3H), 7.52 (s, 1H), 7.45 (d, 1H), 7.26 (t, 1H), 7.12 (d, 1H), 5.98 (d, 1H), 3.83 (q, 1H), 2.27 (d, 6H), 1.50 (d, 3H).

### Example 34. Synthesis of (S)-4-amino-1-(2,6-dichloro-4-methoxyphenyl)-6-oxo-N-(5-(piperidin-2-yl)pyridin-3-yl)-1,6-dihydropyrimidine-5-carboxamide

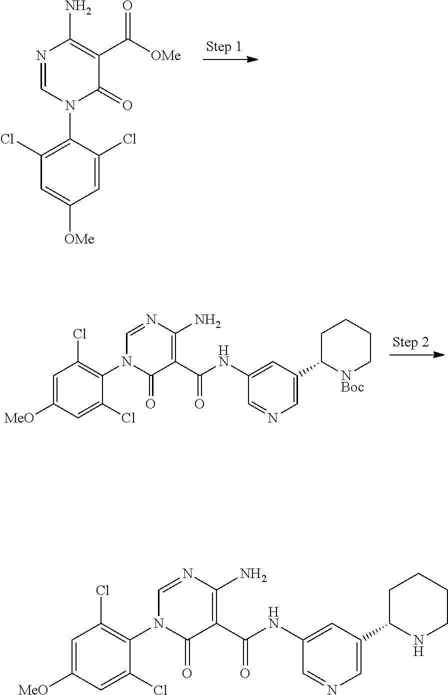 [SMILES: COC(=O)c1c(N)ncn(-c2c(Cl)cc(OC)cc2Cl)c1=O ; COc1cc(Cl)c(-n2cnc(N)c(C(=O)Nc3cncc([C@@H]4CCCCN4C(=O)OC(C)(C)C)c3)c2=O)c(Cl)c1 ; COc1cc(Cl)c(-n2cnc(N)c(C(=O)Nc3cncc([C@@H]4CCCCN4)c3)c2=O)c(Cl)c1]

### Step 1. Synthesis of tert-butyl(S)-2-(5-(4-amino-1-(2,6-dichloro-4-methoxyphenyl)-6-oxo-1,6-dihydropyrimidine-5-carboxamido)pyridin-3-yl) piperidine-1-carboxylate

To a mixture of methyl 4-amino-1-(2,6-dichloro-4-methoxyphenyl)-6-oxo-1,6-dihydropyrimidine-5-carboxylate (Intermediate B10, 30 mg, 0.087 mmol) and tert-butyl(S)-2-(5-aminopyridin-3-yl) piperidine-1-carboxylate (Intermediate A5, 48.4 mg, 0.174 mmol) in toluene (2 mL) was added AlMe₃ (2 M in toluene, 130 μL) and the mixture stirred at 0° C. for 5 min and then at 100° C. for 0.5 h under N₂. The reaction mixture was quenched by addition IN NaOH aq. (4 mL) at 0° C. and extracted with EtOAc (4×5 mL). The combined organics were washed with brine (5 mL), dried (Na₂SO₄) and evaporated to dryness in vacuo to give the title compound as a black oil (40 mg, crude) which was used without further purification. LCMS m/z=589 [M+H]⁺.

### Step 2. Synthesis of (S)-4-amino-1-(2,6-dichloro-4-methoxyphenyl)-6-oxo-N-(5-(piperidin-2-yl)pyridin-3-yl)-1,6-dihydropyrimidine-5-carboxamide

tert-butyl(S)-2-(5-(4-amino-1-(2,6-dichloro-4-methoxyphenyl)-6-oxo-1,6-dihydropyrimidine-5-carboxamido)pyridin-3-yl) piperidine-1-carboxylate (Step 1, 40 mg, 0.068 mmol) was dissolved into DCM (1 mL) and TFA (1 mL) and the mixture stirred at 20° C. for 0.5 h. The reaction mixture was concentrated under reduced pressure and the residue purified by prep-HPLC-1 (1-50% MeCN) to give the title compound as a white solid (16.5 mg, 45%). LCMS m/z=489 [M+H]⁺; ¹H NMR (400 MHz, CDCl₃): 11.78 (s, 1H), 10.03 (br d, 1H), 8.67 (d, 1H), 8.37 (s, 1H), 8.24 (s, 1H), 7.73 (s, 1H), 7.07 (s, 2H), 6.00 (br d, 1H), 3.88 (s, 3H), 3.76-3.69 (m, 1H), 3.19 (br d, 1H), 2.87-2.70 (m, 2H), 1.95-1.82 (m, 2H), 1.73-1.62 (m, 3H), 1.57-1.46 (m, 1H).

### Example 35. Synthesis of (R)-4-amino-N-(5-(1-aminopropyl)pyridin-3-yl)-1-(4-(methoxymethyl)-2,6-dimethylphenyl)-6-oxo-1,6-dihydropyrimidine-5-carboxamide or (S)-4-amino-N-(5-(1-aminopropyl)pyridin-3-yl)-1-(4-(methoxymethyl)-2,6-dimethylphenyl)-6-oxo-1,6-dihydropyrimidine-5-carboxamide

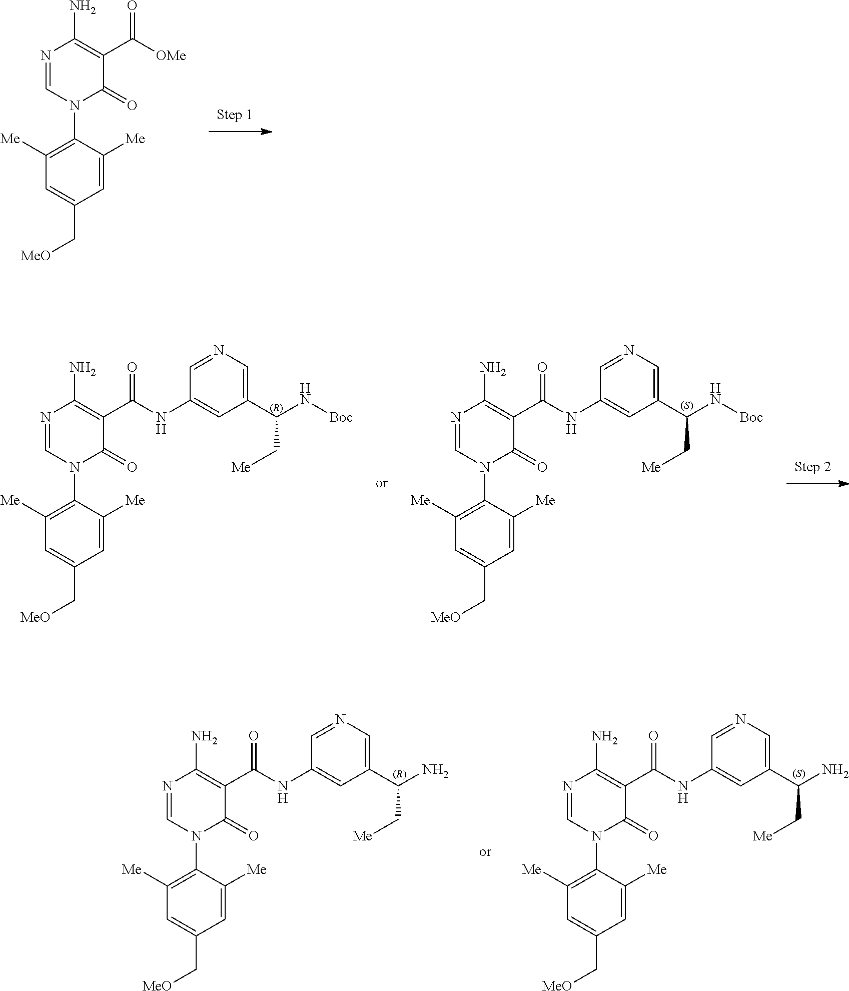

### Step 1. Synthesis of tert-butyl (R)-(1-(5-(4-amino-1-(4-(methoxymethyl)-2,6-dimethylphenyl)-6-oxo-1,6-dihydropyrimidine-5-carboxamido)pyridin-3-yl) propyl)carbamate or tert-butyl(S)-(1-(5-(4-amino-1-(4-(methoxymethyl)-2,6-dimethylphenyl)-6-oxo-1,6-dihydropyrimidine-5-carboxamido)pyridin-3-yl) propyl)carbamate

To a solution of methyl 4-amino-1-(4-(methoxymethyl)-2,6-dimethylphenyl)-6-oxo-1,6-dihydropyrimidine-5-carboxylate (Intermediate B22, 30 mg, 0.095 mmol) and tert-butyl(S)-(1-(5-aminopyridin-3-yl) propyl)carbamate or tert-butyl (R)-(1-(5-aminopyridin-3-yl) propyl)carbamate (Intermediate A8, 47.52 mg, 0.189 mmol) in toluene (4 mL) was added AlMe₃ (2 M, 141 μL) at 0° C. and the mixture stirred at 40° C. for 1 h under N₂. The reaction mixture was quenched by addition of H₂O (0.20 mL) and TFA (0.20 mL) at 0° C. and ten the reaction mixture concentrated under reduced pressure to give the title compound as a yellow oil (40.00 mg, crude). LCMS m/z=537 [M+H]⁺.

### Step 2. Synthesis of (R)-4-amino-N-(5-(1-aminopropyl)pyridin-3-yl)-1-(4-(methoxymethyl)-2,6-dimethylphenyl)-6-oxo-1,6-dihydropyrimidine-5-carboxamide or (S)-4-amino-N-(5-(1-aminopropyl)pyridin-3-yl)-1-(4-(methoxymethyl)-2,6-dimethylphenyl)-6-oxo-1,6-dihydropyrimidine-5-carboxamide

A solution of tert-butyl (R)-(1-(5-(4-amino-1-(4-(methoxymethyl)-2,6-dimethylphenyl)-6-oxo-1,6-dihydropyrimidine-5-carboxamido)pyridin-3-yl) propyl)carbamate or tert-butyl(S)-(1-(5-(4-amino-1-(4-(methoxymethyl)-2,6-dimethylphenyl)-6-oxo-1,6-dihydropyrimidine-5-carboxamido)pyridin-3-yl) propyl)carbamate (Step 1, 40 mg, 0.075 mmol) in TFA (0.50 mL) and DCM (1.50 mL) was stirred at 20° C. for 0.5 h. The reaction mixture was concentrated under N₂ and the residue purified by prep-HPLC-1 (20-50% MeCN) to give the title compound as a white solid (10 mg, 27%). LCMS m/z=537 [M+H]⁺; ¹H NMR (400 MHz, CDCl₃): 0.85 (t, 3H) 1.74-1.89 (m, 2H) 2.18 (d, 6H) 3.45 (s, 3H) 3.93 (t, 1H) 4.46 (s, 2H) 6.04-6.15 (m, 1H) 7.21 (s, 2H) 7.73 (s, 1H) 8.23 (d, 2H) 8.59 (s, 1H) 9.84 (d, 1H) 12.02 (s, 1H).

### Example 36-173

The title compound were prepared from the appropriate amine and ester using an analogous 2-Step method as described for Example 35.

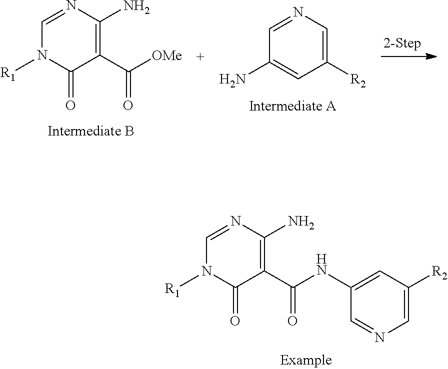

| Example No | Structure, Reactants, HPLC, Data |
|---|---|
| 36 | (S)-4-amino-1-((S)-2-chloro-4-methoxy-6-methylphenyl)-6-oxo-N-(3- |
| | (pyrrolidin-2-yl)phenyl)-1,6-dihydropyrimidine-5-carboxamide |
| | Intermediate B4, Intermediate A61 |
| | HPLC-2 (5-35% MeCN); Pale yellow solid (25.8 mg, 57%); LCMS m/z = |
| | 454 [M + H]⁺; ¹H NMR (400 MHz, CDCl₃): 11.80 (s, 1H), 10.00 (d, 1H), |
| | 7.78-7.69 (m, 2H), 7.46 (d, 1H), 7.27-7.23 (m, 1H), 7.15 (d, 1H), 6.96 (d, |
| | 1H), 6.83 (d, 1H), 6.02 (d, 1H), 4.37 (t, 1H), 3.85 (s, 3H), 3.36-3.04 (m, |
| | 2H), 2.35-2.25 (m, 1H), 2.21 (s, 3H), 2.15-1.91 (m, 3H). |
| 37 | (S)-4-amino-1-(2-chloro-4-fluoro-6-methylphenyl)-6-oxo-N-(3-(pyrrolidin- |
| | 2-yl)phenyl)-1,6-dihydropyrimidine-5-carboxamide |
| | Intermediate B44, Intermediate A61 |
| | HPLC-1 (10-50% MeCN); Orange solid (8.6 mg, 42%); LCMS m/z = 442 |
| | [M + H]⁺; ¹H NMR (400 MHZ, CDCl₃): 11.69 (s, 1H), 10.14 (br.s, 1H), 7.73 |
| | (s, 1H), 7.67-7.53 (m, 2H), 7.24 (dd, 1H), 7.17 (d, 1H), 7.09 (dd, 1H), 5.96 |
| | (br.s, 1H), 4.40-4.00 (m, 1H), 3.30-3.17 (m, 1H), 3.15-2.97 (m, 1H), 2.29 (s, |
| | 3H), 2.25-2.20 (m, 1H), 2.00-1.88 (m, 2H), 1.82-1.73 (m, 1H), 1.38-1.18 |
| | (m, 1H). |
| 38 | 4-amino-N-(3-(1-aminocyclobutyl)phenyl)-1-((S)-2-chloro-4-methoxy-6- |
| | methylphenyl)-6-oxo-1,6-dihydropyrimidine-5-carboxamide |
| | Intermediate B4, Intermediate A70 |
| | HPLC-1 (20-60% MeCN); White solid (28.2 mg, 44%); LCMS m/z = 455 |
| | [M + H]⁺; ¹H NMR (400 MHZ, CDCl₃): 11.76 (s, 1H), 9.91 (d, 1H), 7.71 (s, |
| | 1H), 7.63 (br s, 1H), 7.43 (d, 1H), 7.27 (s, 1H), 7.12 (d, 1H), 6.96 (s, 1H), |
| | 6.82 (br s, 1H), 6.09 (s, 1H), 3.84 (s, 3H), 2.53 (s, 2H), 2.41 (br dd, 2H), |
| | 2.21 (s, 3H), 2.12 (d, 1H), 1.83-1.68 (m, 1H). |
| 39 | (S)-4-amino-1-(2-chloro-4-fluoro-6-methylphenyl)-6-oxo-N-(3-(pyrrolidin- |
| | 2-yl)phenyl)-1,6-dihydropyrimidine-5-carboxamide |
| | Intermediate B44, Intermediate A61 |
| | HPLC-1 (10-45% MeCN); Pale yellow solid (21 mg, 52%); LCMS m/z = |
| | 442 [M + H]⁺; ¹H NMR (400 MHZ, CDCl₃): 11.59 (s, 1H), 10.03 (br d, 1H), |
| | 7.62 (s, 1H), 7.54 (s, 1H), 7.47 (d, 1H), 7.13 (dd, 1H), 7.04 (br d, 1H), 6.98 |
| | (dd, 1H), 5.95 (s, 1H), 4.11 (br t, 1H), 3.16-3.09 (m, 1H), 3.02-2.93 (m, 1H), |
| | 2.18 (s, 3H), 2.16-2.09 (m, 1H), 1.94-1.64 (m, 3H). |
| 40 | (S)-4-amino-1-(2,6-dichlorophenyl)-6-oxo-N-(5-(pyrrolidin-2-yl)pyridin-3- |
| | yl)-1,6-dihydropyrimidine-5-carboxamide |
| | Intermediate B9, Intermediate A13 |
| | HPLC-1 (10-44% MeCN); White solid (85.1 mg, 94%); LCMS m/z = 445 |
| | [M + H]⁺; ¹H NMR (400 MHZ, CDCl₃): 11.77 (s, 1H), 9.95 (br s, 1H), 8.58 |
| | (s, 1H), 8.51-8.50 (m, 1H), 8.42 (s, 1H), 8.37-8.29 (m, 1H), 8.33 (s, 1H), |
| | 7.79-7.70 (m, 1H), 7.61-7.52 (m, 2H), 7.50-7.43 (m, 1H), 7.51-7.43 (m, |
| | 1H), 6.61-6.19 (m, 1H), 4.49 (t, 1H), 3.42-3.19 (m, 2H), 2.37 (d, 1H), 2.25- |
| | 2.00 (m, 3H). |
| 41 | (S)-4-amino-1-((S)-2-chloro-4-cyano-6-methylphenyl)-6-oxo-N-(3- |
| | (piperidin-2-yl)phenyl)-1,6-dihydropyrimidine-5-carboxamide or (R)-4- |
| | amino-1-((S)-2-chloro-4-cyano-6-methylphenyl)-6-oxo-N-(3-(piperidin-2- |
| | yl)phenyl)-1,6-dihydropyrimidine-5-carboxamide |
| | Intermediate B13, Intermediate A20 |
| | HPLC-2 (3-33% MeCN); White solid (4.6 mg, 25%); LCMS m/z = 463 |
| | [M + H]⁺; ¹H NMR (400 MHZ, CDCl₃): 11.55 (s, 1H), 10.13 (d, 1H), 7.75 (d, |
| | 1H), 7.71 (s, 1H), 7.67 (s, 1H), 7.62 (s, 1H), 7.48 (d, 1H), 7.24 (s, 1H), 7.22- |
| | 7.18 (m, 1H), 6.15 (d, 1H), 3.73 (dd, 1H), 3.12 (d, 1H), 2.74 (dt, 1H,), 2.32 |
| | (s, 3H), 1.93-1.82 (m, 3H), 1.74-1.63 (m, 2H), 1.50 (dd, 1H). |
| 42 | (S)-4-amino-N-(3-(1-amino-2-methoxyethyl)phenyl)-1-((S)-2-chloro-4- |
| | cyano-6-methylphenyl)-6-oxo-1,6-dihydropyrimidine-5-carboxamide |
| | Intermediate B13, Intermediate A1 |
| | HPLC-1 (5-40% MeCN); White solid (3.9 mg, 18%); LCMS m/z = 453 |
| | [M + H]⁺; ¹H NMR (400 MHZ, CDCl₃): 11.54 (s, 1H), 10.09 (br d, 1H), 7.75 |
| | (s, 1H), 7.70-7.67 (m, 2H), 7.62 (s, 1H), 7.45 (d, 1H), 7.30-7.27 (m, 1H), |
| | 7.13 (d, 1H), 6.26 (d, 1H), 4.22 (d, 1H), 3.56-3.49 (m, 2H), 3.37 (s, 3H), |
| | 2.32 (s, 3H), 1.21 (d, 1H). |
| 43 | (S)-4-amino-N-(5-(1-aminoethyl)pyridin-3-yl)-1-(2,6-dichloro-4- |
| | cyanophenyl)-6-oxo-1,6-dihydropyrimidine-5-carboxamide or (R)-4-amino- |
| | N-(5-(1-aminoethyl)pyridin-3-yl)-1-(2,6-dichloro-4-cyanophenyl)-6-oxo- |
| | 1,6-dihydropyrimidine-5-carboxamide |
| | Intermediate B14, Intermediate A16 |
| | HPLC-2 (5-35% MeCN); White solid (11.3 mg, 69%); LCMS m/z = 444 |
| | [M + H]⁺; ¹H NMR (400 MHZ, DMSO-d₆): 11.65 (s, 1H), 9.71 (br d, 1H), |
| | 8.86 (br d, 1H), 8.72 (d, 1H), 8.54-8.40 (m, 3H), 8.28 (d, 1H), 8.00 (t, 1H), |
| | 4.06 (q, 1H), 1.29 (d, 3H). |
| 44 | (S)-4-amino-N-(5-(1-amino-2-methoxyethyl)pyridin-3-yl)-1-(2,6-dichloro- |
| | 4-cyanophenyl)-6-oxo-1,6-dihydropyrimidine-5-carboxamide or (R)-4- |
| | amino-N-(5-(1-amino-2-methoxyethyl)pyridin-3-yl)-1-(2,6-dichloro-4- |
| | cyanophenyl)-6-oxo-1,6-dihydropyrimidine-5-carboxamide |
| | Intermediate B14, Intermediate A22 |
| | HPLC-1 (10-45% MeCN); White solid (6.4 mg, 46%); LCMS m/z = 474 |
| | [M + H]⁺; ¹H NMR (400 MHZ, MeOH-d3): 8.85-8.82 (m, 1H), 8.33 (d, 1H), |
| | 8.28 (d, 1H), 8.16 (s, 1H), 8.15 (d, 2H), 4.52 (br dd, 1H), 3.76-3.71 (m, 2H), |
| | 3.45 (s, 3H). |
| 45 | (S)-4-amino-1-(2,6-dichloro-4-methylphenyl)-6-oxo-N-(5-(pyrrolidin-2- |
| | yl)pyridin-3-yl)-1,6-dihydropyrimidine-5-carboxamide |
| | Intermediate B12, Intermediate A13 |
| | HPLC-2 (5-35% MeCN); White solid (19.4 mg, 86%); LCMS m/z = 459 |
| | [M + H]⁺; ¹H NMR (400 MHZ, CDCl₃): 11.78 (s, 1H ), 10.01 (d, 1H), 8.65 |
| | (d, 1H), 8.34 (d, 1H), 8.23-8.27 (m, 1H ), 7.72 (s, 1H ), 7.35 (s, 2H ), 6.16 |
| | (d, 1H), 4.31 (t, 1H), 3.20-3.31 (m, 1H), 3.09-3.18 (m, 1H), 2.43 (s, 3H), |
| | 2.24-2.33 (m, 1H), 2.00-2.08 (m, 1H), 1.86-1.98 (m, 2H). |
| 46 | (S)-4-amino-1-(2,6-dichloro-4-(difluoromethoxy)phenyl)-6-oxo-N-(5- |
| | (piperidin-2-yl)pyridin-3-yl)-1,6-dihydropyrimidine-5-carboxamide |
| | Intermediate B45, Intermediate A5 |
| | HPLC-1 (30-80% MeCN); White solid (18.1 mg, 56%); LCMS m/z = 525 |
| | [M + H]⁺; ¹H NMR (400 MHZ, CDCl₃): 11.90 (s, 1H), 9.82 (d, 1H), 8.69 (s, |
| | 1H), 8.62 (s, 1H), 8.45 (s, 1H), 7.71 (s, 1H), 7.34 (s, 2H), 6.81-6.43 (m, 1H), |
| | 6.20 (d, 1H), 4.13 (d, 1H), 3.36 (d, 1H), 3.02-2.93 (m, 1H), 2.13 (q, 1H), |
| | 2.06-1.90 (m, 3H), 1.87-1.78 (m, 1H), 1.68-1.57 (m, 1H). |
| 47 | (R)-4-amino-N-(5-(1-amino-2-methoxyethyl)pyridin-3-yl)-1-(2,6-dichloro- |
| | 4-(difluoromethoxy)phenyl)-6-oxo-1,6-dihydropyrimidine-5-carboxamide |
| | or (S)-4-amino-N-(5-(1-amino-2-methoxyethyl)pyridin-3-yl)-1-(2,6- |
| | dichloro-4-(difluoromethoxy)phenyl)-6-oxo-1,6-dihydropyrimidine-5- |
| | carboxamide |
| | Intermediate B45, Intermediate A22 |
| | HPLC-2 (5-35% MeCN); White solid (10.7 mg, 58%); LCMS m/z = 515 |
| | [M + H]⁺; ¹H NMR (400 MHZ, DMSO-d₆): 11.76 (s, 1H), 9.66 (br d, 1H), |
| | 8.80-8.73 (m, 2H), 8.44 (s, 1H), 8.27 (br s, 1H), 8.00 (br s, 1H), 7.72 (s, |
| | 2H), 7.67-7.30 (m, 1H), 4.10 (br s, 1H), 3.41 (br s, 2H), 3.25 (s, 3H). |
| 48 | (S)-4-amino-6-oxo-N-(5-(piperidin-2-yl)pyridin-3-yl)-1-(2,4,6- |
| | trichlorophenyl)-1,6-dihydropyrimidine-5-carboxamide |
| | Intermediate B15, Intermediate A5 |
| | HPLC-1 (5-40% MeCN); White solid (43.9 mg, 95%); LCMS m/z = 495 |
| | [M + H]⁺; ¹H NMR (400 MHZ, CDCl₃): 11.70 (s, 1H), 10.01 (d, 1H), 8.58 (s, |
| | 1H), 8.38 (d, 3H), 7.71 (s, 1H), 7.58 (s, 2H), 6.34 (s, 1H), 3.91-3.82 (m, |
| | 1H), 3.22 (d, 1H), 2.88-2.77 (m, 1H), 2.01-1.85 (m, 3H), 1.81-1.69 (m, 2H), |
| | 1.63-1.49 (m, 1H). |
| 49 | (R)-4-amino-N-(5-(1-amino-2-methoxyethyl)pyridin-3-yl)-6-oxo-1-(2,4,6- |
| | trichlorophenyl)-1,6-dihydropyrimidine-5-carboxamide or (S)-4-amino-N- |
| | (5-(1-amino-2-methoxyethyl)pyridin-3-yl)-6-oxo-1-(2,4,6-trichlorophenyl)- |
| | 1,6-dihydropyrimidine-5-carboxamide |
| | Intermediate B15, Intermediate A22 |
| | HPLC-4 (1-40% MeCN); Yellow solid (9.2 mg, 26%); LCMS m/z = 484 |
| | [M + H]⁺; ¹H NMR (400 MHZ, CDCl₃): 11.67 (s, 1H), 10.07 (s, 1H), 8.69 (d, |
| | 1H), 8.30-8.36 (m, 1H), 8.20 (s, 1H), 7.71 (s, 1H), 7.57 (s, 2H), 6.14 (d, |
| | 1H), 4.24 (dd, 1H), 3.48-3.57 (m, 1H), 3.40-3.45 (m, 1H), 3.38-3.40 (m, |
| | 3H). |
| 50 | (R)-4-amino-N-(5-(1-aminoethyl)pyridin-3-yl)-6-oxo-1-(2,4,6- |
| | trichlorophenyl)-1,6-dihydropyrimidine-5-carboxamide or (S)-4-amino-N- |
| | (5-(1-aminoethyl)pyridin-3-yl)-6-oxo-1-(2,4,6-trichlorophenyl)-1,6- |
| | dihydropyrimidine-5-carboxamide |
| | Intermediate B15, Intermediate A16 |
| | HPLC-1 (1-40% MeCN); Yellow oil (16.4 mg, 50%); LCMS m/z = 453 |
| | [M + H]⁺; ¹H NMR (400 MHZ, CDCl₃): 11.61 (s, 1 H), 9.97 (d, 1H), 8.57 (d, |
| | 1H), 8.24 (d, 1H), 8.10-8.17 (m, 1H), 7.63 (s, 1H), 7.50 (s, 1H), 7.45 (s, |
| | 1H), 6.08 (d, 1H), 4.14 (q, 1H), 1.38 (d, 3H). |
| 51 | (R)-4-amino-N-(5-(1-aminoethyl)pyridin-3-yl)-1-(2,6-dichloro-4- |
| | cyclopropoxyphenyl)-6-oxo-1,6-dihydropyrimidine-5-carboxamide or (S)-4- |
| | amino-N-(5-(1-aminoethyl)pyridin-3-yl)-1-(2,6-dichloro-4- |
| | cyclopropoxyphenyl)-6-oxo-1,6-dihydropyrimidine-5-carboxamide |
| | Intermediate B11, Intermediate A16 |
| | HPLC-1 (10-40% MeCN); Yellow solid (18.1 mg, 40%); LCMS m/z = 475 |
| | [M + H]⁺; ¹H NMR (400 MHZ, CDCl₃): 0.82-0.86 (m, 2 H), 0.88 (d, 2H), |
| | 1.52 (d, 3H), 3.79 (tt, 1H), 4.27 (dd, 1H), 6.12-6.37 (m, 1H), 7.21 (s, 2H), |
| | 7.72 (s, 1H), 8.29 (s, 2H), 8.56 (s, 1H), 9.89 (d, 1H), 11.81 (s, 1H). |
| 52 | 4-amino-N-(5-(aminomethyl)pyridin-3-yl)-1-(2,6-dichloro-4- |
| | cyclopropoxyphenyl)-6-oxo-1,6-dihydropyrimidine-5-carboxamide |
| | Intermediate B11, Intermediate A50 |
| | HPLC-3 (15-45% MeCN); White solid (7 mg, 17%); LCMS m/z = 461 |
| | [M + H]⁺; ¹H NMR (400 MHZ, CDCl₃): 11.84-11.72 (m, 1H), 9.96-9.78 (m, |
| | 1H), 8.56-8.44 (m, 1H), 8.32-8.20 (m, 2H), 7.72 (s, 1H), 7.16 (s, 2H), 6.52- |
| | 6.16 (m, 1H), 4.16-3.84 (m, 2H), 3.76 (d, 1H), 0.92-0.86 (m, 2H), 0.84 (s, |
| | 2H). |
| 53 | (R)-4-amino-N-(5-(azepan-2-yl)pyridin-3-yl)-1-(4-methoxy-2,6- |
| | dimethylphenyl)-6-oxo-1,6-dihydropyrimidine-5-carboxamide or (S)-4- |
| | amino-N-(5-(azepan-2-yl)pyridin-3-yl)-1-(4-methoxy-2,6-dimethylphenyl)- |
| | 6-oxo-1,6-dihydropyrimidine-5-carboxamide |
| | Intermediate B18, Intermediate A29 |
| | HPLC-4 (1-35% MeCN); White solid (12.8 mg, 42%); LCMS m/z = 463 |
| | [M + H]⁺; ¹H NMR (400 MHZ, CDCl₃): 12.06 (s, 1H), 9.91 (s, 1H), 8.62 (s, |
| | 1H), 8.33 (s, 1H), 8.27 (s, 1H), 7.75 (s, 1H), 6.75 (s, 2H), 6.01 (s, 1H), 3.95- |
| | 4.01 (m, 1H), 3.83 (s, 3H), 3.15 (d, 1H), 2.90-2.98 (m, 1H), 2.15 (s, 6H), |
| | 2.01 (s, 2H), 1.89 (s, 2H), 1.78 (s, 2H), 1.61-1.71 (m, 2H). |
| 54 | (S)-4-amino-1-(2,6-dichloro-4-(difluoromethyl)phenyl)-6-oxo-N-(3- |
| | (pyrrolidin-2-yl)phenyl)-1,6-dihydropyrimidine-5-carboxamide |
| | Intermediate B16, Intermediate A61 |
| | HPLC-1 (10-50% MeCN); White solid (3.4 mg, 16%); LCMS m/z = 494 |
| | [M + H]⁺; ¹H NMR (400 MHZ, CDCl₃): 11.56 (s, 1H), 10.15 (d, 1H), 7.81- |
| | 7.69 (m, 4H), 7.52 (d, 1H), 7.32 (s, 1H), 7.19 (br d, 1H), 6.93-6.50 (m, 1H), |
| | 6.10 (d, 1H), 4.40 (d, 1H), 3.43-3.09 (m, 2H), 2.39-2.22 (m, 1H), 2.20-1.88 |
| | (m, 3H). |
| 55 | (S)-4-amino-1-(2,6-dichloro-4-(difluoromethyl)phenyl)-6-oxo-N-(5- |
| | (pyrrolidin-2-yl)pyridin-3-yl)-1,6-dihydropyrimidine-5-carboxamide |
| | Intermediate B16, Intermediate A13 |
| | HPLC-1 (10-50% MeCN); White solid (3.4 mg, 20%); LCMS m/z = 495 |
| | [M + H]⁺; ¹H NMR (400 MHZ, DMSO-d₆): 11.75 (s, 1H), 9.67 (d, 1H), 8.83 |
| | (d, 1H), 8.76 (d, 1H), 8.49 (s, 1H), 8.31 (d, 1H), 8.23 (s, 1H), 8.07 (s, 1H), |
| | 8.03 (s, 2H), 7.36-6.96 (m, 1H), 4.28 (t, 1H), 3.21-3.00 (m, 2H), 2.27-2.20 |
| | (m, 1H), 1.93-1.81 (m, 2H), 1.77-1.66 (m, 1H). |
| 56 | (S)-4-amino-1-(2,6-dichloro-4-(difluoromethyl)phenyl)-6-oxo-N-(5- |
| | (piperidin-2-yl)pyridin-3-yl)-1,6-dihydropyrimidine-5-carboxamide |
| | Intermediate B16, Intermediate A5 |
| | HPLC-1 (1-50% MeCN); White solid (21.2 mg, 84%); LCMS m/z = 509 |
| | [M + H]⁺; ¹H NMR (400 MHZ, CDCl₃): 11.66 (s, 1H), 10.07 (d, 1H), 8.61 (d, |
| | 1H), 8.40-8.32 (m, 3H), 7.71 (d, 3H), 6.84-6.54 (m, 1H), 6.20 (d, 1H), 3.80 |
| | (dd, 1H), 3.17 (d, 1H), 2.79 (td, 1H), 2.07-1.61 (m, 6H), 1.60-1.46 (m, 1H). |
| 57 | (R)-4-amino-N-(5-(1-aminoethyl)pyridin-3-yl)-1-(2,6-dichloro-4- |
| | (difluoromethyl)phenyl)-6-oxo-1,6-dihydropyrimidine-5-carboxamide or |
| | (S)-4-amino-N-(5-(1-aminoethyl)pyridin-3-yl)-1-(2,6-dichloro-4- |
| | (difluoromethyl)phenyl)-6-oxo-1,6-dihydropyrimidine-5-carboxamide |
| | Intermediate B16, Intermediate A17 |
| | HPLC-1 (3-33% MeCN); White solid (12.4 mg, 68%); LCMS m/z = 469 |
| | [M + H]⁺; ¹H NMR (400 MHZ, DMSO-d₆): 11.74 (s, 1 H) 9.68 (br d, 1H) |
| | 8.83 (br d, 1H) 8.75 (d, 1H) 8.49 (s, 1 H) 8.27-8.34 (m, 1 H) 6.96-7.37 (m, 1 |
| | H) 4.11 (q, 1H) 1.31 (d, 3H). |
| 58 | (R)-4-amino-N-(5-(1-amino-2-methoxyethyl)pyridin-3-yl)-1-(2,6-dichloro- |
| | 4-(difluoromethyl)phenyl)-6-oxo-1,6-dihydropyrimidine-5-carboxamide or |
| | (S)-4-amino-N-(5-(1-amino-2-methoxyethyl)pyridin-3-yl)-1-(2,6-dichloro- |
| | 4-(difluoromethyl)phenyl)-6-oxo-1,6-dihydropyrimidine-5-carboxamide |
| | Intermediate B16, Intermediate A22 |
| | HPLC-2 (1-35% MeCN); Yellow solid (9.2 mg, 50%); LCMS m/z = 499 |
| | [M + H]⁺; ¹H NMR (400 MHZ, MeOH-d₄): 8.83 (s, 1H), 8.31 (s, 1H), 8.21 (s, |
| | 1H), 8.15 (s, 1H), 7.86 (s, 2H), 7.06-6.77 (m, 1H), 4.39 (t, 1H), 3.71-3.62 |
| | (m, 2H), 3.42 (s, 3H). |
| 59 | (R)-N-(5-(1,4-oxazepan-3-yl)pyridin-3-yl)-4-amino-1-(2,6-dichloro-4- |
| | (difluoromethyl)phenyl)-6-oxo-1,6-dihydropyrimidine-5-carboxamide or |
| | (S)-N-(5-(1,4-oxazepan-3-yl)pyridin-3-yl)-4-amino-1-(2,6-dichloro-4- |
| | (difluoromethyl)phenyl)-6-oxo-1,6-dihydropyrimidine-5-carboxamide |
| | Intermediate B16, Intermediate A27 |
| | HPLC-3 (1-40% MeCN); White solid (25.1 mg, 91%); LCMS m/z = 525 |
| | [M + H]⁺; ¹H NMR (400 MHZ, CDCl₃): 11.60 (s, 1H), 9.96 (s, 1H), 8.60 (s, |
| | 1H), 8.29-8.14 (m, 2H), 7.68-7.60 (m, 3H), 6.61 (t, 1H), 6.21 (s, 1H), 4.02 |
| | (d, 1H), 3.88 (d, 2H), 3.76-3.76 (m, 1H), 3.78 (d, 1H), 3.63 (d, 1H), 3.16 (d, |
| | 1H), 3.05-2.92 (m, 1H), 2.13-1.87 (m, 2H). |
| 60 | 4-amino-1-(2,6-dichloro-4-(methoxymethyl)phenyl)-N-(5- |
| | ((ethylamino)methyl)pyridin-3-yl)-6-oxo-1,6-dihydropyrimidine-5- |
| | carboxamide |
| | Intermediate B19, Intermediate A24 |
| | HPLC-1 (15-45% MeCN); Yellow solid (14.7 mg, 44%); LCMS m/z = 477 |
| | [M + H]⁺; ¹H NMR (400 MHZ, CDCl₃): 10.05-9.94 (m, 1H), 8.58 (d, 1H), |
| | 8.20 (d, 1H), 8.06 (d, 1H), 7.64 (s, 1H), 7.44 (s, 2H), 5.97 (d, 1H), 4.43 (s, |
| | 2H), 3.73 (s, 2H), 3.40 (s, 3H), 2.60 (d, 2H), 1.05 (t, 3H). |
| 61 | (S)-4-amino-1-(2,6-dichloro-4-(methoxymethyl)phenyl)-6-oxo-N-(5- |
| | (piperidin-2-yl)pyridin-3-yl)-1,6-dihydropyrimidine-5-carboxamide |
| | Intermediate B19, Intermediate A5 |
| | HPLC-1 (10-40% MeCN); Yellow solid (4.2 mg, 30%); LCMS m/z = 503 |
| | [M + H]⁺; ¹H NMR (400 MHZ, CDCl₃): 11.87 (s, 1H), 9.88 (d, 1H), 8.63 (s, |
| | 1H), 8.54 (s, 1H), 8.34 (s, 1H), 7.71 (s, 1H), 7.51 (s, 2H), 6.09 (d, 1H), 4.51 |
| | (s, 2H), 4.09-4.02 (m, 1H), 3.48 (s, 3H), 3.30-3.21 (m, 1H), 2.96-2.86 (m, |
| | 1H), 2.15-2.08 (m, 1H), 2.07-1.97 (m, 4H), 1.69-1.55 (m, 1H). |
| 62 | (R)-4-amino-N-(5-(1-amino-2-methoxyethyl)pyridin-3-yl)-1-(2,6-dichloro- |
| | 4-(methoxymethyl)phenyl)-6-oxo-1,6-dihydropyrimidine-5-carboxamide or |
| | (S)-4-amino-N-(5-(1-amino-2-methoxyethyl)pyridin-3-yl)-1-(2,6-dichloro- |
| | 4-(methoxymethyl)phenyl)-6-oxo-1,6-dihydropyrimidine-5-carboxamide |
| | Intermediate B19, Intermediate A22 |
| | HPLC-1 (10-40% MeCN); White solid (18.3 mg, 50%); LCMS m/z = 493 |
| | [M + H]⁺; ¹H NMR (400 MHZ, CDCl₃): 11.80 (s, 1H), 9.83 (d, 1H), 8.52 (s, |
| | 1H), 8.44 (s, 1H), 8.30 (s, 1H), 7.73 (s, 1H), 7.50 (s, 2H), 6.40 (d, 1H), 4.50 |
| | (s, 2H), 4.41 (s, 1H), 3.73 (t, 1H), 3.65-3.60 (m, 1H), 3.46 (s, 3H), 3.39 (s, |
| | 3H). |
| 63 | 4-amino-1-(2,6-dichloro-4-(methoxymethyl)phenyl)-N-(5-(((2- |
| | hydroxyethyl)amino)methyl)pyridin-3-yl)-6-oxo-1,6-dihydropyrimidine-5- |
| | carboxamide |
| | Intermediate B19, Intermediate A71 |
| | HPLC-1 (1-30% MeCN); Yellow solid (16.3 mg, 72%); LCMS m/z = 493 |
| | [M + H]⁺; ¹H NMR (400 MHz, CDCl₃): 11.79 (s, 1H), 9.94 (s, 1H), 8.57 (s, |
| | 1H), 8.39 (s, 1H), 8.31 (s, 1H), 7.70 (s, 1H), 7.49 (s, 2H), 6.47 (s, 1H), 4.49 |
| | (s, 2H), 4.02 (s, 2H), 3.76 (s, 2H), 3.46 (s, 3H), 2.94 (s, 2H). |
| 64 | 4-amino-1-(2,6-dichloro-4-(methoxymethyl)phenyl)-N-(5- |
| | ((isopropylamino)methyl)pyridin-3-yl)-6-oxo-1,6-dihydropyrimidine-5- |
| | carboxamide |
| | Intermediate B19, Intermediate A25 |
| | HPLC-1 (1-40% MeCN); White solid (28.3 mg, 67%); LCMS m/z = 491 |
| | [M + H]⁺; ¹H NMR (400 MHZ, CDCl₃): 11.66 (s, 1H), 9.95 (d, 1H), 8.55 (d, |
| | 1H), 8.21 (d, 2H), 7.64 (s, 1H), 7.44 (s, 2H), 6.03 (d, 1H), 4.43 (s, 2H), 3.79 |
| | (s, 2H), 3.40 (s, 3H), 2.88 (td, 1H), 1.08 (d, 6H). |
| 65 | 4-amino-N-(5-(1-aminocyclobutyl)pyridin-3-yl)-1-(2,6-dichloro-4- |
| | (methoxymethyl)phenyl)-6-oxo-1,6-dihydropyrimidine-5-carboxamide |
| | Intermediate B19, Intermediate A51 |
| | HPLC-1 (15-45% MeCN); Yellow gum (11.2 mg, 30%); LCMS m/z = 489 |
| | [M + H]⁺; ¹H NMR (400 MHZ, DMSO-d₆): 11.93 (s, 1H), 9.64 (s, 1H), 8.93 |
| | (s, 1H), 8.85 (d, 1H), 8.47 (d, 2H), 8.12 (d, 1H), 7.71 (s, 2H), 4.55 (s, 2H), |
| | 3.39 (s, 3H), 2.66-2.59 (m, 2H), 2.48-2.42 (m, 2H), 2.18-2.09 (m, 1H), 1.87- |
| | 1.77 (m, 1H). |
| 66 | (R)-4-amino-N-(5-(1-aminoethyl)pyridin-3-yl)-1-(2,6-dichloro-4- |
| | (methoxymethyl)phenyl)-6-oxo-1,6-dihydropyrimidine-5-carboxamide or |
| | (S)-4-amino-N-(5-(1-aminoethyl)pyridin-3-yl)-1-(2,6-dichloro-4- |
| | (methoxymethyl)phenyl)-6-oxo-1,6-dihydropyrimidine-5-carboxamide |
| | Intermediate B19, Intermediate A17 |
| | HPLC-1 (1-40% MeCN); Yellow solid (11.4 mg, 30%); LCMS m/z = 463 |
| | [M + H]⁺; ¹H NMR (400 MHZ, CDCl₃): 11.67 (s, 1H), 9.94 (d, 1H), 8.57 (s, |
| | 1H), 8.24 (s, 1H), 8.13 (s, 1H), 7.64 (s, 1H), 7.44 (s, 2H), 6.02 (d, 1H ), 4.43 |
| | (s, 2H), 4.15 (d, 1H), 3.40 (s, 3H), 1.43-1.38 (m, 3H). |
| 67 | (S)-4-amino-N-(5-(1-aminoethyl)pyridin-3-yl)-1-(2,6-dichloro-4- |
| | (methoxymethyl)phenyl)-6-oxo-1,6-dihydropyrimidine-5-carboxamide or |
| | (R)-4-amino-N-(5-(1-aminoethyl)pyridin-3-yl)-1-(2,6-dichloro-4- |
| | (methoxymethyl)phenyl)-6-oxo-1,6-dihydropyrimidine-5-carboxamide |
| | Intermediate B19, Intermediate A16 |
| | HPLC-1 (1-40% MeCN); Yellow solid (11.4 mg, 31%); LCMS m/z = 463 |
| | [M + H]⁺; ¹H NMR (400 MHZ, CDCl₃): 11.77 (s, 1H), 9.87 (d, 1H), 8.64- |
| | 8.43 (m, 1H), 8.40-8.16 (m, 2H), 7.72 (s, 1H), 7.51 (s, 2H), 7.28 (s, 3H), |
| | 6.39-6.24 (m, 1H), 4.51 (s, 2H), 4.43-4.27 (m, 1H), 3.48 (s, 3H), 1.56 (s, |
| | 3H). |
| 68 | (S)-4-amino-1-(2,6-dichloro-4-(methoxymethyl)phenyl)-N-(5-(2-methoxy- |
| | 1-(methylamino)ethyl)pyridin-3-yl)-6-oxo-1,6-dihydropyrimidine-5- |
| | carboxamide or (R)-4-amino-1-(2,6-dichloro-4-(methoxymethyl)phenyl)-N- |
| | (5-(2-methoxy-1-(methylamino)ethyl)pyridin-3-yl)-6-oxo-1,6- |
| | dihydropyrimidine-5-carboxamide |
| | Intermediate B19, Intermediate A23 |
| | HPLC-1 (15-55% MeCN); White solid (16.5 mg, 36%); LCMS m/z = 507 |
| | [M + H]⁺; ¹H NMR (400 MHZ, CDCl₃): 11.82 (s, 1H), 10.02 (d, 1H), 8.69 (s, |
| | 1H), 8.41 (s, H), 8.33 - 8.38 (m, 1H), 7.72 (s, 1H), 7.52 (s, 2H), 6.11 (d, |
| | 1H), 4.51 (s, 2H), 3.98-4.16 (m, 1H), 3.70-3.79 (m, 2H), 3.62-3.70 (m, 1H), |
| | 3.48 (s, 3H), 3.39 (s, 3H), 2.49 (s, 3H). |
| 69 | (R)-4-amino-N-(5-(1-amino-2-ethoxyethyl)pyridin-3-yl)-1-(2,6-dichloro-4- |
| | (methoxymethyl)phenyl)-6-oxo-1,6-dihydropyrimidine-5-carboxamide or |
| | (S)-4-amino-N-(5-(1-amino-2-ethoxyethyl)pyridin-3-yl)-1-(2,6-dichloro-4- |
| | (methoxymethyl)phenyl)-6-oxo-1,6-dihydropyrimidine-5-carboxamide |
| | Intermediate B19, Intermediate A10 |
| | HPLC-1 (20-60% MeCN); White solid (15.3 mg, 40%); LCMS m/z = 507 |
| | [M + H]⁺; ¹H NMR (400 MHZ, CDCl₃): 11.80 (s, 1H), 9.94 (d, 1H), 8.62 (d, |
| | 1H), 8.36 (d, 2H), 7.73 (s, 1H), 7.53 (s, 2H), 6.25 (d, 1H), 4.52 (s, 2H), 4.34 |
| | (dd, 1H), 3.68-3.60 (m, 2H), 3.57 (ddd, 2H), 3.49 (s, 3H), 1.21 (t, 3H). |
| 70 | (R)-4-amino-N-(5-(1-amino-3,3-difluoropropyl)pyridin-3-yl)-1-(2,6- |
| | dichloro-4-(methoxymethyl)phenyl)-6-oxo-1,6-dihydropyrimidine-5- |
| | carboxamide or (S)-4-amino-N-(5-(1-amino-3,3-difluoropropyl)pyridin-3- |
| | yl)-1-(2,6-dichloro-4-(methoxymethyl)phenyl)-6-oxo-1,6- |
| | dihydropyrimidine-5-carboxamide |
| | Intermediate B19, Intermediate A44 |
| | HPLC-1 (5-35% MeCN); White solid (24.7 mg, 53%); LCMS m/z = 513 |
| | [M + H]⁺; ¹H NMR (400 MHZ, CDCl₃): 11.83 (s, 1H), 10.04 (d, 1H), 8.68 (d, |
| | 1H), 8.31 (d, 1H), 8.24 (s, 1H), 7.74 (s, 1H), 7.54 (s, 2H), 6.18 (d, 1H), 5.93 |
| | (tt, 1H), 4.52 (s, 2H), 4.26 (dd, 1H), 3.49 (s, 3H), 2.27-2.18 (m, 2H). |
| 71 | (R)-4-amino-1-((S)-2-chloro-4-ethoxy-6-methylphenyl)-N-(3-(1- |
| | (methylamino)ethyl)phenyl)-6-oxo-1,6-dihydropyrimidine-5-carboxamide |
| | Intermediate B5, Intermediate A2 |
| | HPLC-1 (1-40% MeCN); White solid (50.2 mg, 70%); LCMS m/z = 456 |
| | [M + H]⁺; ¹H NMR (400 MHZ, CDCl₃): 11.83 (s, 1H), 10.04 (d, 1H), 7.73 (s, |
| | 1H), 7.66 (s, 1H), 7.53 (d, 1H), 7.31 (t, 1H), 7.11 (d, 1H), 6.95 (d, 1H), 6.81 |
| | (d, 1H), 6.04 (d, 1H), 4.06 (q, 2H), 3.82 (q, 1H), 2.34 (s, 3H), 2.20 (s, 3H), |
| | 1.50 (d, 3H), 1.44 (t, 3H). |
| 72 | (S)-4-amino-N-(3-(1-amino-2-methoxyethyl)phenyl)-1-((S)-2-chloro-4- |
| | ethoxy-6-methylphenyl)-6-oxo-1,6-dihydropyrimidine-5-carboxamide |
| | Intermediate B5, Intermediate Al |
| | HPLC-1 (5-40% MeCN); Pale yellow solid (7.9 mg, 11%); LCMS m/z = |
| | 472 [M + H]⁺; ¹H NMR (400 MHZ, CDCl₃): 11.79 (s, 1H), 10.01 (d, 1H), |
| | 7.72 (s, 1H), 7.67 (s, 1H), 7.52 (d, 1H), 7.30 (s, 1H), 7.10 (d, 1H), 6.95 (d, |
| | 1H), 6.81 (d, 1H), 5.97 (d, 1H), 4.19 (dd, 1H), 4.06 (q, 2H), 3.56-3.51 (m, |
| | 1H), 3.48-3.43 (m, 1H), 3.38 (s, 3H), 2.20 (s, 3H), 1.45 (t, 3H). |
| 73 | (S)-4-amino-1-(2,6-dichloro-4-methoxyphenyl)-6-oxo-N-(5-(pyrrolidin-2- |
| | yl)pyridin-3-yl)-1,6-dihydropyrimidine-5-carboxamide |
| | Intermediate B10, Intermediate A13 |
| | HPLC-1 (10-50% MeCN); White solid (3.6 mg, 13%); LCMS m/z = 475 |
| | [M + H]⁺; ¹H NMR (400 MHZ, DMSO-d₆): 11.90 (s, 1H), 9.63 (d, 1H), 8.79 |
| | (d, 1H), 8.76 (d, 1H), 8.43 (s, 1H), 8.32 (s, 1H), 8.10 (s, 1H), 7.40 (s, 1H), |
| | 7.42-7.37 (m, 1H), 4.32 (t, 1H), 3.91 (s, 3H), 3.22-2.99 (m, 2H), 2.32-2.17 |
| | (m, 1H), 1.99-1.90 (m, 1H), 1.89-1.66 (m, 2H). |
| 74 | (R)-4-amino-N-(5-(1-aminoethyl)pyridin-3-yl)-1-(2,6-dichloro-4- |
| | methoxyphenyl)-6-oxo-1,6-dihydropyrimidine-5-carboxamide or (S)-4- |
| | amino-N-(5-(1-aminoethyl)pyridin-3-yl)-1-(2,6-dichloro-4-methoxyphenyl)- |
| | 6-oxo-1,6-dihydropyrimidine-5-carboxamide |
| | Intermediate B10, Intermediate A17 |
| | HPLC-1 (5-35% MeCN); Yellow oil (14.1 mg, 57%); LCMS m/z = 449 |
| | [M + H]⁺; ¹H NMR (400 MHZ, DMSO-d₆): 11.83 (s, 1H), 9.59 (s, 1H), 8.72 |
| | (d, 2H), 8.38 (s, 1H), 8.26 (s, 1H), 8.00 (s, 1H), 7.35 (s, 2H), 4.09 (d, 1H), |
| | 3.86 (s, 3H), 1.29 (d, 3H). |
| 75 | (R)-N-(5-(1,4-oxazepan-3-yl)pyridin-3-yl)-4-amino-1-(2,6-dichloro-4- |
| | methoxyphenyl)-6-oxo-1,6-dihydropyrimidine-5-carboxamide or (S)-N-(5- |
| | (1,4-oxazepan-3-yl)pyridin-3-yl)-4-amino-1-(2,6-dichloro-4- |
| | methoxyphenyl)-6-oxo-1,6-dihydropyrimidine-5-carboxamide |
| | Intermediate B10, Intermediate A27 |
| | HPLC-1 (5-40% MeCN); White solid (11.8 mg, 28%); LCMS m/z = 505 |
| | [M + H]⁺; ¹H NMR (400 MHZ, CDCl₃): 11.71 (s, 1H), 9.95 (br d, 1H), 8.65 |
| | (d, 1H), 8.23 (s, 1H), 8.08 (s, 1H), 7.66 (s, 1H), 6.99 (s, 2H), 5.97 (br d, |
| | 1H), 3.95-3.84 (m, 3H), 3.80 (s, 3H), 3.80-3.73 (m, 1H), 3.45 (dd, 1H), 3.14 |
| | (dt, 1H), 2.94 (ddd, 1H), 1.92-1.89 (m, 2H). |
| 76 | (S)-4-amino-1-(2,6-dichloro-4-methoxyphenyl)-N-(6-fluoro-5-(piperidin-2- |
| | yl)pyridin-3-yl)-6-oxo-1,6-dihydropyrimidine-5-carboxamide or (R)-4- |
| | amino-1-(2,6-dichloro-4-methoxyphenyl)-N-(6-fluoro-5-(piperidin-2- |
| | yl)pyridin-3-yl)-6-oxo-1,6-dihydropyrimidine-5-carboxamide |
| | Intermediate B10, Intermediate A21 |
| | HPLC-2 (5-40% MeCN); White solid (30 mg, 65%); LCMS m/z = 507 |
| | [M + H]⁺; ¹H NMR (400 MHZ, CDCl₃): 11.75 (s, 1H), 9.97 (br d, 1H), 8.42 |
| | (dd, 1H), 8.30-8.27 (m, 1H), 7.72 (s, 1H), 7.06 (s, 2H), 6.14 (br d, 1H), |
| | 4.02-3.95 (m, 1H), 3.88 (s, 3H), 3.27 (br d, 1H), 2.83 (td, 1H), 1.91 (br d, |
| | 2H, J = 10.4 Hz), 1.70-1.50 (m, 4H). |
| 77 | (S)-4-amino-N-(5-(1-amino-2-methoxyethyl)pyridin-3-yl)-1-(2,6-dichloro- |
| | 4-methoxyphenyl)-6-oxo-1,6-dihydropyrimidine-5-carboxamide |
| | Intermediate B10, Intermediate A22 |
| | HPLC-1 (1-40% MeCN); Yellow solid (57.7 mg, 79%); LCMS m/z = 479 |
| | [M + H]⁺; ¹H NMR (400 MHZ, DMSO-d₆): 11.83 (s, 1H), 9.63 (d, 1H), 8.74 |
| | (d, 1H), 8.72 (d, 1H), 8.40 (s, 1H), 8.26 (d, 1H), 8.00 (d, 1H), 7.37 (s, 2H), |
| | 4.08 (t, 1H), 3.89 (s, 3H), 3.38 (d, 2H), 3.24 (s, 3H). |
| 78 | 4-amino-1-(2,6-dichloro-4-methoxyphenyl)-N-(5-((2R, 5R)-5- |
| | methylpyrrolidin-2-yl)pyridin-3-yl)-6-oxo-1,6-dihydropyrimidine-5- |
| | carboxamide or 4-amino-1-(2,6-dichloro-4-methoxyphenyl)-N-(5-((2S, 5S)- |
| | 5-methylpyrrolidin-2-yl)pyridin-3-yl)-6-oxo-1,6-dihydropyrimidine-5- |
| | carboxamide or 4-amino-1-(2,6-dichloro-4-methoxyphenyl)-N-(5-((2R, 5S)- |
| | 5-methylpyrrolidin-2-yl)pyridin-3-yl)-6-oxo-1,6-dihydropyrimidine-5- |
| | carboxamide or 4-amino-1-(2,6-dichloro-4-methoxyphenyl)-N-(5-((2S, 5R)- |
| | 5-methylpyrrolidin-2-yl)pyridin-3-yl)-6-oxo-1,6-dihydropyrimidine-5- |
| | carboxamide |
| | Intermediate B10, Intermediate A35 |
| | HPLC-1 (15-55% MeCN); White solid (50.5 mg, 93%); LCMS m/z = 489 |
| | [M + H]⁺; ¹H NMR (400 MHZ, CDCl₃): 11.80 (s, 1H), 9.99 (d, 1H), 8.64 (d, |
| | 1H), 8.39-8.25 (m, 2H), 7.74 (s, 1H), 7.08 (s, 2H), 6.15 (d, 1H), 4.40 (t, 1H), |
| | 3.89 (s, 3H), 3.61-3.46 (m, 1H), 2.37-2.25 (m, 1H), 2.20-2.09 (m, 1H), 2.09- |
| | 1.96 (m, 1H), 1.82-1.61 (m, 1H), 1.29 (d, 3H). |
| 79 | 4-amino-1-(2,6-dichloro-4-methoxyphenyl)-N-(5-((2R, 5S)-5- |
| | methylpyrrolidin-2-yl)pyridin-3-yl)-6-oxo-1,6-dihydropyrimidine-5- |
| | carboxamide or 4-amino-1-(2,6-dichloro-4-methoxyphenyl)-N-(5-((2S, 5R)- |
| | 5-methylpyrrolidin-2-yl)pyridin-3-yl)-6-oxo-1,6-dihydropyrimidine-5- |
| | carboxamide or 4-amino-1-(2,6-dichloro-4-methoxyphenyl)-N-(5-((2R, 5R)- |
| | 5-methylpyrrolidin-2-yl)pyridin-3-yl)-6-oxo-1,6-dihydropyrimidine-5- |
| | carboxamide or 4-amino-1-(2,6-dichloro-4-methoxyphenyl)-N-(5-((2S, 5S)- |
| | 5-methylpyrrolidin-2-yl)pyridin-3-yl)-6-oxo-1,6-dihydropyrimidine-5- |
| | carboxamide |
| | Intermediate B10, Intermediate A38 |
| | HPLC-3 (30-65% MeCN); Yellow solid (8.8 mg, 17%); LCMS m/z = 489 |
| | [M + H]⁺; ¹H NMR (400 MHZ, CDCl₃): 12.11 (s, 1H), 9.81 (d, 1H), 8.80- |
| | 9.05 (m, 1H), 8.40-8.63 (m, 2H), 7.73 (s, 1H), 6.98-7.10 (m, 2H), 6.15 (d, |
| | 1H), 4.80-4.99 (m, 1H), 3.85-4.00 (m, 4H), 2.39-2.52 (m, 2H), 2.27-2.38 |
| | (m, 1H), 1.76-1.96 (m, 1H), 1.44 (d, 3H). |
| 80 | (R)-4-amino-1-(2,6-dichloro-4-methoxyphenyl)-N-(5-(2-methoxy-1- |
| | (methylamino)ethyl)pyridin-3-yl)-6-oxo-1,6-dihydropyrimidine-5- |
| | carboxamide or (S)-4-amino-1-(2,6-dichloro-4-methoxyphenyl)-N-(5-(2- |
| | methoxy-1-(methylamino)ethyl)pyridin-3-yl)-6-oxo-1,6-dihydropyrimidine- |
| | 5-carboxamide |
| | Intermediate B10, Intermediate A23 |
| | HPLC1-1 (05-36% MeCN); White solid (45.5 mg, 71%); LCMS m/z = 493 |
| | [M + H]⁺; ¹H NMR (400 MHZ, CDCl₃): 11.79 (s, 1H), 10.06 (br d, 1H), 8.74 |
| | (d, 1H), 8.29 (d, 1H), 8.14 (t, 1H), 7.74 (s, 1H), 7.07 (s, 2H), 6.11 (d, 1H), |
| | 3.88 (s, 3H), 3.80 (dd, 1H), 3.49-3.42 (m, 2H), 3.37 (s, 3H), 2.31 (s, 3H). |
| 81 | 4-amino-1-(2,6-dichloro-4-methoxyphenyl)-N-(5-((2R, 6S)-6- |
| | methylpiperidin-2-yl)pyridin-3-yl)-6-oxo-1,6-dihydropyrimidine-5- |
| | carboxamide or 4-amino-1-(2,6-dichloro-4-methoxyphenyl)-N-(5-((2S,6R)- |
| | 6-methylpiperidin-2-yl)pyridin-3-yl)-6-oxo-1,6-dihydropyrimidine-5- |
| | carboxamide or 4-amino-1-(2,6-dichloro-4-methoxyphenyl)-N-(5-((2R, 6R)- |
| | 6-methylpiperidin-2-yl)pyridin-3-yl)-6-oxo-1,6-dihydropyrimidine-5- |
| | carboxamide or 4-amino-1-(2,6-dichloro-4-methoxyphenyl)-N-(5-((2S,6S)- |
| | 6-methylpiperidin-2-yl)pyridin-3-yl)-6-oxo-1,6-dihydropyrimidine-5- |
| | carboxamide |
| | Intermediate B10, Intermediate A33 |
| | HPLC-1 (1-40% MeCN); White solid (33 mg, 98%); LCMS m/z = 503 |
| | [M + H]⁺; ¹H NMR (400 MHz, CDCl₃): 11.79 (s, 1H), 9.99 (d, 1H), 8.57 (d, |
| | 1H), 8.41-8.39 (m, 1H), 8.36 (d, 1H), 7.72 (s, 1H), 7.06 (s, 2H), 6.12 (d, |
| | 1H), 4.18 (dd, 1H), 3.88 (s, 3H), 3.42-3.38 (m, 1H), 1.97-1.88 (m, 3H), |
| | 1.80-1.67 (m, 2H), 1.51-1.47 (m, 1H), 1.27 (d, 3H). |
| 82 | (R)-4-amino-N-(5-(1-amino-3,3-difluoropropyl)pyridin-3-yl)-1-(2,6- |
| | dichloro-4-methoxyphenyl)-6-oxo-1,6-dihydropyrimidine-5-carboxamide or |
| | (S)-4-amino-N-(5-(1-amino-3,3-difluoropropyl)pyridin-3-yl)-1-(2,6- |
| | dichloro-4-methoxyphenyl)-6-oxo-1,6-dihydropyrimidine-5-carboxamide |
| | Intermediate B10, Intermediate A44 |
| | HPLC-1 (1-40% MeCN); White solid (22.1 mg, 39%); LCMS m/z = 499 |
| | [M + H]⁺; ¹H NMR (400 MHz, CDCl₃): 11.86 (s, 1H), 9.98 (d, 1H), 8.65 (s, |
| | 1H), 8.32-8.21 (m, 2H), 7.74 (d, 1H), 7.07 (d, 2H), 6.14 (d, 1H), 6.08-5.74 |
| | (m, 1H), 4.26 (t, 1H), 3.88 (d, 3H), 2.36-2.24 (m, 2H). |
| 83 | 4-amino-1-(2,6-dichloro-4-methoxyphenyl)-N-(5-((2R, 6R)-6- |
| | methylpiperidin-2-yl)pyridin-3-yl)-6-oxo-1,6-dihydropyrimidine-5- |
| | carboxamide or 4-amino-1-(2,6-dichloro-4-methoxyphenyl)-N-(5-((2S,6S)- |
| | 6-methylpiperidin-2-yl)pyridin-3-yl)-6-oxo-1,6-dihydropyrimidine-5- |
| | carboxamide or 4-amino-1-(2,6-dichloro-4-methoxyphenyl)-N-(5-((2R, 6S)- |
| | 6-methylpiperidin-2-yl)pyridin-3-yl)-6-oxo-1,6-dihydropyrimidine-5- |
| | carboxamide or 4-amino-1-(2,6-dichloro-4-methoxyphenyl)-N-(5-((2S,6R)- |
| | 6-methylpiperidin-2-yl)pyridin-3-yl)-6-oxo-1,6-dihydropyrimidine-5- |
| | carboxamide |
| | Intermediate B10, Intermediate A32 |
| | HPLC-1 (5-35% MeCN); White solid (18.6 mg, 39%); LCMS m/z = 503 |
| | [M + H]⁺; ¹H NMR (400 MHZ, CDCl₃): 1.05 (d, 3H), 1.21-1.29 (m, 1H), |
| | 1.47-1.63 (m, 2H), 1.67 (d, 1H), 1.79 (d, 1H), 1.87-1.93 (m, 1H), 2.83-2.90 |
| | (m, 1H) 3.73-3.79 (m, 1H), 3.88 (s, 3H), 6.06 (s, 1H), 7.07 (s, 2H), 7.73 (s, |
| | 1H), 8.17 (s, 1H), 8.34 (s, 1H), 8.72 (d, 1H), 10.04 (d, 1H), 11.76 (s, 1H). |
| 84 | 4-amino-1-(2,6-dichloro-4-methoxyphenyl)-N-(5-((2R, 6R)-6- |
| | methylpiperidin-2-yl)pyridin-3-yl)-6-oxo-1,6-dihydropyrimidine-5- |
| | carboxamide or 4-amino-1-(2,6-dichloro-4-methoxyphenyl)-N-(5-((2S,6S)- |
| | 6-methylpiperidin-2-yl)pyridin-3-yl)-6-oxo-1,6-dihydropyrimidine-5- |
| | carboxamide or 4-amino-1-(2,6-dichloro-4-methoxyphenyl)-N-(5-((2R, 6S)- |
| | 6-methylpiperidin-2-yl)pyridin-3-yl)-6-oxo-1,6-dihydropyrimidine-5- |
| | carboxamide or 4-amino-1-(2,6-dichloro-4-methoxyphenyl)-N-(5-((2S,6R)- |
| | 6-methylpiperidin-2-yl)pyridin-3-yl)-6-oxo-1,6-dihydropyrimidine-5- |
| | carboxamide |
| | Intermediate B10, Intermediate A31 |
| | HPLC-1 (1-40% MeCN); White solid (20.6 mg, 50%); LCMS m/z = 503 |
| | [M + H]⁺; ¹H NMR (400 MHZ, CDCl₃): 11.79 (s, 1H), 10.01 (d, 1H), 8.58 (d, |
| | 1H), 8.39 (d, 2H), 7.72 (s, 1H), 7.06 (s, 2H), 6.10 (d, 1H), 4.17 ( dd, 1H), |
| | 3.88 (s, 3H), 3.45-3.33 (m, 1H), 2.03-1.87 (m, 3H), 1.81-1.66 (m, 2H), 1.55- |
| | 1.44 (m, 1H), 1.27 (d, 3H). |
| 85 | (R)-4-amino-N-(5-(1-amino-3,3-difluoropropyl)pyridin-3-yl)-1-(2,6- |
| | dichloro-4-methoxyphenyl)-6-oxo-1,6-dihydropyrimidine-5-carboxamide or |
| | (S)-4-amino-N-(5-(1-amino-3,3-difluoropropyl)pyridin-3-yl)-1-(2,6- |
| | dichloro-4-methoxyphenyl)-6-oxo-1,6-dihydropyrimidine-5-carboxamide |
| | Intermediate B10, Intermediate A44 |
| | HPLC-1 (10-40% MeCN); White solid (8.7 mg, 23%); LCMS m/z = 499 |
| | [M + H]⁺; ¹H NMR (400 MHZ, CDCl₃): 11.86 (s, 1H), 10.01 (d, 1H), 8.67 (d, |
| | 1H), 8.30 (d, 1H), 8.23 (s, 1H), 7.74 (s, 1H), 7.07 (s, 2H), 6.06-6.03(m, 1H), |
| | 5.94-5.75 (m, 1H), 4.24 (dd, 1H), 3.88 (s, 3H), 2.29-2.16 (m, 2H). |
| 86 | 4-amino-1-(2,6-dichloro-4-propoxyphenyl)-N-(5- |
| | ((ethylamino)methyl)pyridin-3-yl)-6-oxo-1,6-dihydropyrimidine-5- |
| | carboxamide |
| | Intermediate B7, Intermediate A24 |
| | HPLC-3 (5-35% MeCN); White solid (23.7 mg, 48%); LCMS m/z = 491 |
| | [M + H]⁺; ¹H NMR (400 MHZ, CDCl₃): 11.73 (s, 1H), 9.93 (br.d, 1H), 8.56 |
| | (d, 1H), 8.21 (s, 1H), 8.15 (br.s, 1H), 7.65 (s, 1H), 6.98 (s, 2H), 6.07 (br.d, |
| | 1H), 3.89 (t, 2H), 3.77 (s, 2H), 2.64 (q, 2H), 1.77 (sxt, 2H), 1.08 (br.t, 3H), |
| | 0.98 (t, 3H). |
| 87 | 4-amino-1-(2,6-dimethyl-4-(trifluoromethyl)phenyl)-N-(5-((2R, 5S)-5- |
| | methylpyrrolidin-2-yl)pyridin-3-yl)-6-oxo-1,6-dihydropyrimidine-5- |
| | carboxamide or 4-amino-1-(2,6-dimethyl-4-(trifluoromethyl)phenyl)-N-(5- |
| | ((2S, 5R)-5-methylpyrrolidin-2-yl)pyridin-3-yl)-6-oxo-1,6- |
| | dihydropyrimidine-5-carboxamide or 4-amino-1-(2,6-dimethyl-4- |
| | (trifluoromethyl)phenyl)-N-(5-((2R, 5R)-5-methylpyrrolidin-2-yl)pyridin-3- |
| | yl)-6-oxo-1,6-dihydropyrimidine-5-carboxamide or 4-amino-1-(2,6- |
| | dimethyl-4-(trifluoromethyl)phenyl)-N-(5-((2S, 5S)-5-methylpyrrolidin-2- |
| | yl)pyridin-3-yl)-6-oxo-1,6-dihydropyrimidine-5-carboxamide |
| | Intermediate B24, Intermediate A37 |
| | HPLC-1 (1-40% MeCN); Yellow solid (21.9 mg, 65%); LCMS m/z = 487 |
| | [M + H]⁺; ¹H NMR (400 MHZ, CDCl₃): 11.82 (s, 1H), 10.03 (d, 1H), 8.69 (d, |
| | 1H), 8.30 (s, 1H), 8.12 (s, 1H), 7.71 (s, 1H), 7.51 (s, 2H), 6.10 (d, 1H), 4.44 |
| | (t, 1H), 3.55-3.50 (m, 1H), 2.37-2.33 (m, 1H), 2.26 (s, 6H), 2.20-2.09 (m, |
| | 1H), 1.85-1.78 (m, 1H), 1.52-1.45 (m, 1H), 1.24 (d, 3H). |
| 88 | (R)-4-amino-N-(5-(1-aminopropyl)pyridin-3-yl)-1-(2,6-dimethyl-4- |
| | (trifluoromethyl)phenyl)-6-oxo-1,6-dihydropyrimidine-5-carboxamide or |
| | (S)-4-amino-N-(5-(1-aminopropyl)pyridin-3-yl)-1-(2,6-dimethyl-4- |
| | (trifluoromethyl)phenyl)-6-oxo-1,6-dihydropyrimidine-5-carboxamide |
| | Intermediate B24, Intermediate A8 |
| | HPLC-1 (5-35% MeCN); White solid (22.5 mg, 34%); LCMS m/z = 461 |
| | [M + H]⁺; ¹H NMR (400 MHZ, CDCl₃): 11.90 (s, 1H), 10.00 (d, 1H), 8.66 (d, |
| | 1H), 8.29 (s, 1H), 8.22 (s, 1H), 7.74 (s, 1H), 7.54 (s, 2H), 6.26 (d, 1H), 3.94 |
| | (t, 1H), 2.29 (d, 6H), 1.85-1.75 (m, 2H), 0.90 (t, 3H). |
| 89 | (R)-4-amino-N-(5-(azepan-2-yl)pyridin-3-yl)-1-(2,6-dimethyl-4- |
| | (trifluoromethyl)phenyl)-6-oxo-1,6-dihydropyrimidine-5-carboxamide or |
| | (S)-4-amino-N-(5-(azepan-2-yl)pyridin-3-yl)-1-(2,6-dimethyl-4- |
| | (trifluoromethyl)phenyl)-6-oxo-1,6-dihydropyrimidine-5-carboxamide |
| | Intermediate B24, Intermediate A29 |
| | HPLC-1 (10-50% MeCN); Brown oil (32.2 mg, 63%); LCMS m/z = 501 |
| | [M + H]⁺; ¹H NMR (400 MHZ, CDCl₃): 11.89 (s, 1H), 10.01 (d, 1H), 8.63 (d, |
| | 1H), 8.41-8.27 (m, 2H), 7.74 (s, 1H), 7.55 (s, 2H), 6.14 (d, 1H), 4.12-3.96 |
| | (m, 1H), 3.21-3.09 (m, 1H), 3.05-2.89 (m, 1H), 2.29 (s, 6H), 2.11-2.01 (m, |
| | 2H), 1.98-1.88 (m, 2H), 1.86-1.75 (m, 2H), 1.75-1.59 (m, 2H). |
| 90 | 4-amino-1-(2,6-dimethyl-4-(trifluoromethyl)phenyl)-N-(5-((2R, 6S)-6- |
| | methylpiperidin-2-yl)pyridin-3-yl)-6-oxo-1,6-dihydropyrimidine-5- |
| | carboxamide or 4-amino-1-(2,6-dimethyl-4-(trifluoromethyl)phenyl)-N-(5- |
| | ((2S,6R)-6-methylpiperidin-2-yl)pyridin-3-yl)-6-oxo-1,6- |
| | dihydropyrimidine-5-carboxamide or 4-amino-1-(2,6-dimethyl-4- |
| | (trifluoromethyl)phenyl)-N-(5-((2R, 6R)-6-methylpiperidin-2-yl)pyridin-3- |
| | yl)-6-oxo-1,6-dihydropyrimidine-5-carboxamide or 4-amino-1-(2,6- |
| | dimethyl-4-(trifluoromethyl)phenyl)-N-(5-((2S,6S)-6-methylpiperidin-2- |
| | yl)pyridin-3-yl)-6-oxo-1,6-dihydropyrimidine-5-carboxamide |
| | Intermediate B24, Intermediate A31 |
| | HPLC-1 (10-40% MeCN); Pink solid (38.3 mg, 91%); LCMS m/z = 491 |
| | [M + H]⁺; ¹H NMR (400 MHZ, CDCl₃): 1.28 (d, 3H), 1.42-1.57 (m, 1H), |
| | 1.66-1.84 (m, 2H), 1.84-2.00 (m, 3H), 2.27 (s, 6H), 3.41 (d, 1H), 4.18 (t, |
| | 1H), 6.03 (d, 1H), 7.52 (s, 2H), 7.72 (s, 1H), 8.26-8.47 (m, 2H), 8.63 (s, |
| | 1H), 9.99 (d, 1H), 11.85 (s, 1H). |
| 91 | (R)-4-amino-1-(4-ethoxy-2,6-dimethylphenyl)-6-oxo-N-(5-(piperidin-2- |
| | yl)pyridin-3-yl)-1,6-dihydropyrimidine-5-carboxamide |
| | Intermediate B25, Intermediate A4 |
| | HPLC-4 (1-40% MeCN); White solid (72 mg, 66%); LCMS m/z = 463 |
| | [M + H]⁺; ¹H NMR (400 MHZ, CDCl₃): 12.07 (s, 1H), 9.85 (d, 1H), 8.57 (d, |
| | 1H), 8.35 (d, 2H), 7.74 (s, 1H), 6.72 (s, 2H), 6.32 (d, 1H), 4.04 (q, 2H), 3.81 |
| | (dd, 1H), 3.18 (d, 1H), 2.82-2.74 (m, 1H), 2.12 (s, 6H), 1.92 (d, 1H), 1.89- |
| | 1.81 (m, 2H), 1.74-1.66 (m, 2H), 1.57-1.48 (m, 1H), 1.42 (t, 3H). |
| 92 | 4-amino-1-(4-ethoxy-2,6-dimethylphenyl)-N-(5-((2R, 5S)-5- |
| | methylpyrrolidin-2-yl)pyridin-3-yl)-6-oxo-1,6-dihydropyrimidine-5- |
| | carboxamide or 4-amino-1-(4-ethoxy-2,6-dimethylphenyl)-N-(5-((2S, 5R)-5- |
| | methylpyrrolidin-2-yl)pyridin-3-yl)-6-oxo-1,6-dihydropyrimidine-5- |
| | carboxamide or 4-amino-1-(4-ethoxy-2,6-dimethylphenyl)-N-(5-((2R, 5R)- |
| | 5-methylpyrrolidin-2-yl)pyridin-3-yl)-6-oxo-1,6-dihydropyrimidine-5- |
| | carboxamide or 4-amino-1-(4-ethoxy-2,6-dimethylphenyl)-N-(5-((2S, 5S)-5- |
| | methylpyrrolidin-2-yl)pyridin-3-yl)-6-oxo-1,6-dihydropyrimidine-5- |
| | carboxamide |
| | Intermediate B25, Intermediate A37 |
| | HPLC-1 (1-40% MeCN); White solid (23 mg, 35%); LCMS m/z = 463 |
| | [M + H]⁺; ¹H NMR (400 MHZ, CDCl₃): 12.07 (s, 1H), 9.87 (d, 1H), 8.62 (s, |
| | 1H), 8.30 (d, 2H), 7.75 (s, 1H), 6.73 (s, 2H), 6.01 (d, 1H), 4.59 (t, 1H), 4.05 |
| | (q, 3H), 3.69 (d, 1H), 2.42-2.33 (m, 1H), 2.30-2.19 (m, 1H), 2.13 (d, 6H), |
| | 2.08-1.98 (m, 1H), 1.72-1.59 (m, 1H), 1.43 (t, 3H), 1.30 (d, 3H). |
| 93 | 4-amino-1-(4-ethoxy-2,6-dimethylphenyl)-N-(5-((2R, 5S)-5- |
| | methylpyrrolidin-2-yl)pyridin-3-yl)-6-oxo-1,6-dihydropyrimidine-5- |
| | carboxamide or 4-amino-1-(4-ethoxy-2,6-dimethylphenyl)-N-(5-((2S, 5R)-5- |
| | methylpyrrolidin-2-yl)pyridin-3-yl)-6-oxo-1,6-dihydropyrimidine-5- |
| | carboxamide or 4-amino-1-(4-ethoxy-2,6-dimethylphenyl)-N-(5-((2R, 5R)- |
| | 5-methylpyrrolidin-2-yl)pyridin-3-yl)-6-oxo-1,6-dihydropyrimidine-5- |
| | carboxamide or 4-amino-1-(4-ethoxy-2,6-dimethylphenyl)-N-(5-((2S, 5S)-5- |
| | methylpyrrolidin-2-yl)pyridin-3-yl)-6-oxo-1,6-dihydropyrimidine-5- |
| | carboxamide |
| | Intermediate B25, Intermediate A38 |
| | HPLC-1 (1-40% MeCN); White solid (29.6 mg, 90%); LCMS m/z = 463 |
| | [M + H]⁺; ¹H NMR (400 MHZ, CDCl₃): 1.30 (d, 3 H), 1.47 (t, 3 H), 1.53-1.67 |
| | (m, 1 H), 1.88-2.02 (m, 1 H), 2.17 (s, 6 H), 2.21 (d, 1 H), 2.32-2.45 (m, 1 |
| | H), 3.59-3.69 (m, 1 H), 4.09 (q, 2 H), 4.53 (t, 1 H), 6.02 (d, 1 H), 6.77 (s, 2 |
| | H), 7.78 (s, 1 H), 8.23 (s, 1 H), 8.31-8.38 (m, 1 H), 8.70 (s, 1 H), 9.94 (d, 1 |
| | H), 12.07 (s, 1 H). |
| 94 | (R)-4-amino-N-(5-(1-aminobutyl)pyridin-3-yl)-1-(4-ethoxy-2,6- |
| | dimethylphenyl)-6-oxo-1,6-dihydropyrimidine-5-carboxamide |
| | Intermediate B25, Intermediate A7 |
| | HPLC-1 (20-60% MeCN); White solid (19.8 mg, 30%); LCMS m/z = 451 |
| | [M + H]⁺; ¹H NMR (400 MHZ, CDCl₃): 12.07 (s, 1H), 9.76 (s, 1H), 8.53 (s, |
| | 1H), 8.31-8.21 (m, 2H), 7.74 (s, 1H), 6.73 (s, 2H), 6.20 (d, 1H), 4.05 (q, |
| | 3H), 2.13 (d, 6H), 1.89-1.72 (m, 2H), 1.43 (t, 3H), 1.31-1.14 (m, 2H), 0.87 |
| | (t, 3H). |
| 95 | (R)-4-amino-N-(5-(azepan-2-yl)pyridin-3-yl)-1-(4-ethoxy-2,6- |
| | dimethylphenyl)-6-oxo-1,6-dihydropyrimidine-5-carboxamide or (S)-4- |
| | amino-N-(5-(azepan-2-yl)pyridin-3-yl)-1-(4-ethoxy-2,6-dimethylphenyl)-6- |
| | oxo-1,6-dihydropyrimidine-5-carboxamide |
| | Intermediate B25, Intermediate A29 |
| | HPLC-1 (20-60% MeCN); White solid (19 mg, 38%); LCMS m/z = 477 |
| | [M + H]⁺; ¹H NMR (400 MHZ, CDCl₃): 11.97 (s, 1 H), 9.83 (d, 1H), 8.52 (s, |
| | 1 H), 8.12-8.30 (m, 2 H), 7.67 (s, 1 H), 6.66 (s, 2 H), 5.83 (d, 1H), 3.96-4.07 |
| | (m, 2 H), 3.91-3.95 (m, 1 H), 2.98-3.14 (m, 1 H), 2.82-2.93 (m, 1 H), 2.06 |
| | (s, 6 H), 1.90-2.02 (m, 2 H), 1.78-1.90 (m, 2 H), 1.53-1.75 (m, 4 H), 1.36 (t, |
| | 3H). |
| 96 | 4-amino-1-(4-ethoxy-2,6-dimethylphenyl)-N-(5-((2R, 6S)-6- |
| | methylpiperidin-2-yl)pyridin-3-yl)-6-oxo-1,6-dihydropyrimidine-5- |
| | carboxamide or 4-amino-1-(4-ethoxy-2,6-dimethylphenyl)-N-(5-((2S,6R)-6- |
| | methylpiperidin-2-yl)pyridin-3-yl)-6-oxo-1,6-dihydropyrimidine-5- |
| | carboxamide or 4-amino-1-(4-ethoxy-2,6-dimethylphenyl)-N-(5-((2R, 6R)- |
| | 6-methylpiperidin-2-yl)pyridin-3-yl)-6-oxo-1,6-dihydropyrimidine-5- |
| | carboxamide or 4-amino-1-(4-ethoxy-2,6-dimethylphenyl)-N-(5-((2S,6S)-6- |
| | methylpiperidin-2-yl)pyridin-3-yl)-6-oxo-1,6-dihydropyrimidine-5- |
| | carboxamide |
| | Intermediate B25, Intermediate A31 |
| | HPLC-1 (10-40% MeCN); White solid (51 mg, 85%); LCMS m/z = 477 |
| | [M + H]⁺; ¹H NMR (400 MHZ, CDCl₃): 11.96 (s, 1H), 9.83 (d, 1H), 8.52 (d, |
| | 1H), 8.29 (d, 2H), 7.67 (s, 1H), 6.65 (s, 2H), 5.90 (d, 1H), 4.08 (dd, 1H), |
| | 3.98 (q, 2H), 3.38-3.22 (m, 1H), 2.05 (s, 6H), 1.92-1.77 (m, 3H), 1.72-1.56 |
| | (m, 2H), 1.41 (d, 1H), 1.36 (t, 3H), 1.18 (d, 3H). |
| 97 | (R)-4-amino-1-(4-(difluoromethyl)-2,6-dimethylphenyl)-6-oxo-N-(5- |
| | (piperidin-2-yl)pyridin-3-yl)-1,6-dihydropyrimidine-5-carboxamide |
| | Intermediate B17, Intermediate A4 |
| | HPLC-1 (15-45% MeCN); White solid (33.6 mg, 81%); LCMS m/z = 469 |
| | [M + H]⁺; ¹H NMR (400 MHZ, CDCl₃): 11.90 (s, 1H), 9.89 (d, 1H), 8.63- |
| | 8.41 (m, 2H), 8.28 (d, 1H), 7.73 (s, 1H), 7.40 (s, 2H), 6.84-6.49 (m, 1H), |
| | 6.09 (d, 1H), 3.92 (dd, 1H), 3.21 (d, 1H), 2.86 (dt, 1H), 2.25 (d, 6H), 2.04- |
| | 1.89 (m, 3H), 1.88-1.69 (m, 2H), 1.65-1.48 (m, 1H). |
| 98 | (R)-4-amino-1-(4-(methoxymethyl)-2,6-dimethylphenyl)-6-oxo-N-(5- |
| | (piperidin-2-yl)pyridin-3-yl)-1,6-dihydropyrimidine-5-carboxamide |
| | Intermediate B22, Intermediate A4 |
| | HPLC-1 (15-55% MeCN); White solid (21.8 mg, 81%); LCMS m/z = 463 |
| | [M + H]⁺; ¹H NMR (400 MHZ, CDCl₃): 12.02 (s, 1 H) 9.88 (s, 1 H) 8.48 (s, 1 |
| | H) 7.72 (s, 1 H) 7.21 (s, 2 H) 6.03 (s, 1 H) 4.46 (s, 2 H) 3.83-3.96 (m, 1 H) |
| | 3.45 (s, 3 H) 3.16 (d, 1 H) 2.73-2.87 (m, 1 H) 2.18 (d, 6 H) 1.88-2.02 (m, 3 |
| | H) 1.68-1.85 (m, 2 H) 1.49-1.60 (m, 1 H). |
| 99 | 4-amino-1-(4-(methoxymethyl)-2,6-dimethylphenyl)-N-(5- |
| | ((methylamino)methyl)pyridin-3-yl)-6-oxo-1,6-dihydropyrimidine-5- |
| | carboxamide |
| | Intermediate B22, Intermediate A6 |
| | HPLC-1 (5-35% MeCN); White solid (34.6 mg, 64%); LCMS m/z = 423 |
| | [M + H]⁺; ¹H NMR (400 MHZ, CDCl₃): 12.07 (s, 1H), 9.82 (br s, 1H), 8.55 |
| | (br s, 1H), 8.46 (br s, 1H), 8.31 (br s, 1H), 7.71 (s, 1H), 7.19 (s, 2H), 6.49 |
| | (br s, 1H), 4.44 (s, 2H), 3.98 (br s, 2H), 3.43 (s, 3H), 2.50 (br s, 3H), 2.15 (s, |
| | 6H). |
| 100 | (R)-4-amino-N-(5-(azepan-2-yl)pyridin-3-yl)-1-(4-(methoxymethyl)-2,6- |
| | dimethylphenyl)-6-oxo-1,6-dihydropyrimidine-5-carboxamide or (S)-4- |
| | amino-N-(5-(azepan-2-yl)pyridin-3-yl)-1-(4-(methoxymethyl)-2,6- |
| | dimethylphenyl)-6-oxo-1,6-dihydropyrimidine-5-carboxamide |
| | Intermediate B22, Intermediate A30 |
| | HPLC-1 (1-40% MeCN); White solid (20.5 mg, 55%); LCMS m/z = 477 |
| | [M + H]⁺; ¹H NMR (400 MHZ, CDCl₃): 11.92 (s, 1H), 9.85 (d, 1H), 8.53 (d, |
| | 1H), 8.22 (d, 2H), 7.65 (s, 1H), 7.13 (s, 2H), 5.88 (d, 1H), 4.39 (s, 2H), 3.89 |
| | (t, 1H), 3.37 (s, 3H), 3.03-3.01 (m, 1H), 2.87-2.82 (m, 1H), 2.10 (s, |
| | 6H), 1.95-1.91 (m, 2H), 1.84-1.79 (m, 2H), 1.71-1.50 (m, 4H). |
| 101 | 4-amino-1-(4-(methoxymethyl)-2,6-dimethylphenyl)-N-(5-((2R, 5S)-5- |
| | methylpyrrolidin-2-yl)pyridin-3-yl)-6-oxo-1,6-dihydropyrimidine-5- |
| | carboxamide or 4-amino-1-(4-(methoxymethyl)-2,6-dimethylphenyl)-N-(5- |
| | ((2S, 5R)-5-methylpyrrolidin-2-yl)pyridin-3-yl)-6-oxo-1,6- |
| | dihydropyrimidine-5-carboxamide or 4-amino-1-(4-(methoxymethyl)-2,6- |
| | dimethylphenyl)-N-(5-((2R, 5R)-5-methylpyrrolidin-2-yl)pyridin-3-yl)-6- |
| | ox0-1,6-dihydropyrimidine-5-carboxamide or 4-amino-1-(4- |
| | (methoxymethyl)-2,6-dimethylphenyl)-N-(5-((2S, 5S)-5-methylpyrrolidin-2- |
| | yl)pyridin-3-yl)-6-oxo-1,6-dihydropyrimidine-5-carboxamide |
| | Intermediate B22, Intermediate A38 |
| | HPLC-1 (10-40% MeCN); White solid (31.7 mg, 77%); LCMS m/z = 463 |
| | [M + H]⁺; ¹H NMR (400 MHZ, CDCl₃): 1.31 (d, 3H), 1.63-1.79 (m, 1H), |
| | 2.02-2.15 (m, 1H), 2.19 (d, 6H), 2.24-2.34 (m, 1H), 2.35-2.46 (m, 1H), 3.46 |
| | (s, 3H), 3.67-3.82 (m, 1H), 4.48 (s, 2H), 4.64 (dd, 1H), 6.02 (d, 1H), 7.22 (s, |
| | 2H), 7.73 (s, 1H), 8.23-8.41 (m, 2H), 8.63 (d, 1H), 9.89 (d, 1H), 12.03 (s, |
| | 1H). |
| 102 | 4-amino-1-(4-(methoxymethyl)-2,6-dimethylphenyl)-N-(5-((2R, 6S)-6- |
| | methylpiperidin-2-yl)pyridin-3-yl)-6-oxo-1,6-dihydropyrimidine-5- |
| | carboxamide or 4-amino-1-(4-(methoxymethyl)-2,6-dimethylphenyl)-N-(5- |
| | ((2S,6R)-6-methylpiperidin-2-yl)pyridin-3-yl)-6-oxo-1,6- |
| | dihydropyrimidine-5-carboxamideor 4-amino-1-(4-(methoxymethyl)-2,6- |
| | dimethylphenyl)-N-(5-((2R, 6R)-6-methylpiperidin-2-yl)pyridin-3-yl)-6- |
| | oxo-1,6-dihydropyrimidine-5-carboxamide or 4-amino-1-(4- |
| | (methoxymethyl)-2,6-dimethylphenyl)-N-(5-((2S,6S)-6-methylpiperidin-2- |
| | yl)pyridin-3-yl)-6-oxo-1,6-dihydropyrimidine-5-carboxamide |
| | Intermediate B22, Intermediate A34 |
| | HPLC-3 (1-40% MeCN); White solid (33.9 mg, 61%); LCMS m/z = 477 |
| | [M + H]⁺; ¹H NMR (400 MHZ, CDCl₃): 1.27 (d, 3H), 1.49 (d, 1H), 1.67-1.80 |
| | (m, 2H), 1.88-2.07 (m, 3H), 2.17 (s, 6H), 3.45 (s, 3H), 4.18 (d, 1H), 4.46 (s, |
| | 2H), 6.11 (d, 1H), 7.20 (s, 2H), 7.72 (s, 1H), 8.38 (s, 1H), 8.59 (s, 1H), 9.91 |
| | (d, 1H) 12.00 (s, 1H). |
| 103 | 4-amino-1-(4-(methoxymethyl)-2,6-dimethylphenyl)-N-(5-((2R, 5S)-5- |
| | methylpyrrolidin-2-yl)pyridin-3-yl)-6-oxo-1,6-dihydropyrimidine-5- |
| | carboxamide or 4-amino-1-(4-(methoxymethyl)-2,6-dimethylphenyl)-N-(5- |
| | ((2S, 5R)-5-methylpyrrolidin-2-yl)pyridin-3-yl)-6-oxo-1,6- |
| | dihydropyrimidine-5-carboxamide or 4-amino-1-(4-(methoxymethyl)-2,6- |
| | dimethylphenyl)-N-(5-((2R, 5R)-5-methylpyrrolidin-2-yl)pyridin-3-yl)-6- |
| | ox0-1,6-dihydropyrimidine-5-carboxamide or 4-amino-1-(4- |
| | (methoxymethyl)-2,6-dimethylphenyl)-N-(5-((2S, 5S)-5-methylpyrrolidin-2- |
| | yl)pyridin-3-yl)-6-oxo-1,6-dihydropyrimidine-5-carboxamide |
| | Intermediate B22, Intermediate A37 |
| | HPLC-1 (1-40% MeCN); Yellow solid (57.9 mg, 85%); LCMS m/z = 463 |
| | [M + H]⁺; ¹H NMR (400 MHZ, CDCl₃): 1.30 (d, 3H), 1.64-1.78 (m, 1H), |
| | 2.07-2.15 (m, 1H), 2.17 (d, 6H), 2.24-2.33 (m, 1H), 2.34-2.44 (m, 1H), 3.45 |
| | (s, 3H), 3.72-3.82 (m, 1H), 4.46 (s, 2H), 4.64 (d, 1H), 6.09 (d, 1H), 7.20 (s, |
| | 2H), 7.71 (s, 1H), 8.29 (d, 1H), 8.35 (s, 1H), 8.60 (d, 1H), 9.86 (d, 1H), |
| | 12.01 (s, 1H). |
| 104 | (R)-4-amino-N-(5-(1-aminoethyl)pyridin-3-yl)-1-(4-ethoxy-2,6- |
| | dimethylphenyl)-6-oxo-1,6-dihydropyrimidine-5-carboxamide or (S)-4- |
| | amino-N-(5-(1-aminoethyl)pyridin-3-yl)-1-(4-ethoxy-2,6-dimethylphenyl)- |
| | 6-oxo-1,6-dihydropyrimidine-5-carboxamide |
| | Intermediate B25, Intermediate A16 |
| | HPLC-1 (5-35% MeCN); White solid (10.2 mg, 25%); LCMS m/z = 423 |
| | [M + H]⁺; ¹H NMR (400 MHZ, CDCl₃): 11.99 (s, 1H), 9.74 (s, 1H), 8.51 (s, |
| | 1H), 8.20 (s, 2H), 7.67 (s, 1H), 6.65 (s, 2H), 6.08 (d, 1H), 4.15 (d, 1H), 3.97 |
| | (q, 2H), 2.05(d, 6H), 1.41-1.33 (m, 6H). |
| 105 | 4-amino-1-(4-(difluoromethyl)-2,6-dimethylphenyl)-N-(5- |
| | ((methylamino)methyl)pyridin-3-yl)-6-oxo-1,6-dihydropyrimidine-5- |
| | carboxamide |
| | Intermediate B17, Intermediate A56 |
| | HPLC-1 (10-40% MeCN); White solid (60.9 mg, 92%); LCMS m/z = 429 |
| | [M + H]⁺; ¹H NMR (400 MHZ, CDCl₃): 11.99 (s, 1H), 9.93 (d, 1H), 8.60 (s, |
| | 1H), 8.47 (s, 1H), 8.35 (s, 1H), 7.74 (s, 1H), 7.41 (s, 2H), 6.67 (t, 1H), 6.08 |
| | (d, 1H), 4.01 (s, 2H), 2.56 (s, 3H), 2.25 (s, 6H). |
| 106 | (R)-4-amino-N-(5-(1-aminobutyl)pyridin-3-yl)-1-(4-(difluoromethyl)-2,6- |
| | dimethylphenyl)-6-oxo-1,6-dihydropyrimidine-5-carboxamide |
| | Intermediate B17, Intermediate A7 |
| | HPLC-1 (1-40% MeCN); White solid (28.2 mg, 41%); LCMS m/z = 503 |
| | [M + H]⁺; ¹H NMR (400 MHZ, CDCl₃): 11.94 (s, 1H), 9.82 (s, 1H), 8.50 (s, |
| | 1H), 8.34 (s, 1H), 8.25 (s, 1H), 7.71 (s, 1H), 7.38 (s, 2H), 6.84-6.47 (m, 1H), |
| | 6.42 (s, 1H), 4.14-4.02 (m, 1H), 2.23 (d, 6H, J = 8.4 Hz), 1.94-1.74 (m, 2H), |
| | 1.26 (br s, 2H), 0.86 (t, 3H). |
| 107 | 4-amino-1-(4-(difluoromethyl)-2,6-dimethylphenyl)-N-(5-((2R, 6S)-6- |
| | methylpiperidin-2-yl)pyridin-3-yl)-6-oxo-1,6-dihydropyrimidine-5- |
| | carboxamide or 4-amino-1-(4-(difluoromethyl)-2,6-dimethylphenyl)-N-(5- |
| | ((2S,6R)-6-methylpiperidin-2-yl)pyridin-3-yl)-6-oxo-1,6- |
| | dihydropyrimidine-5-carboxamide or 4-amino-1-(4-(difluoromethyl)-2,6- |
| | dimethylphenyl)-N-(5-((2R, 6R)-6-methylpiperidin-2-yl)pyridin-3-yl)-6- |
| | oxo-1,6-dihydropyrimidine-5-carboxamide or 4-amino-1-(4- |
| | (difluoromethyl)-2,6-dimethylphenyl)-N-(5-((2S,6S)-6-methylpiperidin-2- |
| | yl)pyridin-3-yl)-6-oxo-1,6-dihydropyrimidine-5-carboxamide |
| | Intermediate B17, Intermediate A31 |
| | HPLC-1 (5-45% MeCN); White solid (44.6 mg, 98%); LCMS m/z = 483 |
| | [M + H]⁺; ¹H NMR (400 MHZ, CDCl₃): 11.93 (s, 1H), 10.00 (d, 1H), 8.65 (d, |
| | 1H), 8.42 (s, 1H), 8.34 (s, 1H), 7.75 (s, 1H), 7.42 (s, 2H), 6.68 (t, 1H ), 6.23 |
| | (d, 1H), 4.18 (t, 1H), 3.40 (d, 1H), 2.27 (s, 6H), 2.27-1.91 (m, 3H), 1.74- |
| | 1.71 (m, 2H), 1.51-1.47 (m, 1H), 1.29 (d, 3H). |
| 108 | 4-amino-1-(4-(difluoromethyl)-2,6-dimethylphenyl)-N-(5-((2R, 6S)-6- |
| | methylpiperidin-2-yl)pyridin-3-yl)-6-oxo-1,6-dihydropyrimidine-5- |
| | carboxamide or 4-amino-1-(4-(difluoromethyl)-2,6-dimethylphenyl)-N-(5- |
| | ((2S,6R)-6-methylpiperidin-2-yl)pyridin-3-yl)-6-oxo-1,6- |
| | dihydropyrimidine-5-carboxamide or 4-amino-1-(4-(difluoromethyl)-2,6- |
| | dimethylphenyl)-N-(5-((2R, 6R)-6-methylpiperidin-2-yl)pyridin-3-yl)-6- |
| | ox0-1,6-dihydropyrimidine-5-carboxamide or 4-amino-1-(4- |
| | (difluoromethyl)-2,6-dimethylphenyl)-N-(5-((2S,6S)-6-methylpiperidin-2- |
| | yl)pyridin-3-yl)-6-oxo-1,6-dihydropyrimidine-5-carboxamide |
| | Intermediate B17, Intermediate A33 |
| | HPLC-1 (5-35% MeCN); White solid (20.9 mg, 92%); LCMS m/z = 483 |
| | [M + H]⁺; ¹H NMR (400 MHZ, CDCl₃): 11.93 (s, 1H), 10.09-9.90 (m, 1H), |
| | 8.64 (d, 1H), 8.43 (d, 1H), 8.35 (s, 1H), 7.74 (s, 1H), 7.42 (s, 2H), 6.91-6.46 |
| | (m, 1H), 6.11 (d, 1H), 4.24-4.13 (m, 1H), 3.42 (d, 1H), 2.26 (s, 6H), 1.95 (d, |
| | 3H), 1.84-1.67 (m, 2H), 1.57-1.44 (m, 1H), 1.30 (d, 3H). |
| 109 | (S)-4-amino-1-(2,6-dichloro-4-(trifluoromethoxy)phenyl)-6-oxo-N-(5- |
| | (pyrrolidin-2-yl)pyridin-3-yl)-1,6-dihydropyrimidine-5-carboxamide |
| | Intermediate B37, Intermediate A13 |
| | HPLC-2 (10-40% MeCN); Yellow solid (10.1 mg, 60%); LCMS m/z = 529 |
| | [M + H]⁺; ¹H NMR (400 MHZ, CDCl₃): 11.64 (s, 1H), 10.10 (s, 1H), 8.69 (d, |
| | 1H), 8.35 (d, 1H), 8.20 (t, 1H), 7.73 (s, 1H), 7.46 (d, 2H), 6.12 (d, 1H), 4.28 |
| | (t, 1H), 3.25-3.23 (m, 1H), 3.15-3.09 (m, 1H), 2.06-1.94 (m, 2H), 1.92-1.89 |
| | (m, 1H), 1.86-1.76 (m, 1H). |
| 110 | (R)-4-amino-1-(2,6-dimethyl-4-(trifluoromethoxy)phenyl)-6-oxo-N-(5- |
| | (piperidin-2-yl)pyridin-3-yl)-1,6-dihydropyrimidine-5-carboxamide |
| | Intermediate B38, Intermediate A4 |
| | HPLC-1 (1-40% MeCN); White solid (21.2 mg, 66%); LCMS m/z = 503 |
| | [M + H]⁺; ¹H NMR (400 MHZ, CDCl₃): 1.26 (s, 1 H) , 1.48-1.58 (m, 1 H), |
| | 1.70-1.79 (m, 2 H), 1.85-1.96 (m, 2 H), 2.21 (d, 6 H), 2.81 (dd, 1 H), 3.20 |
| | (br.d, 1 H), 3.78 (dd, 1 H), 5.97 (br.d, 1 H), 7.10 (s, 2 H), 7.72 (s, 1 H), |
| | 8.29-8.35 (m, 2 H), 8.63 (d, 1 H), 9.97 (d, 1 H) , 11.83-11.93 (m, 1 H). |
| 111 | 4-amino-1-(4-((R)-1,2-difluoroethyl)-2,6-dimethylphenyl)-6-oxo-N-(5-((R)- |
| | piperidin-2-yl)pyridin-3-yl)-1,6-dihydropyrimidine-5-carboxamide or 4- |
| | amino-1-(4-((S)-1,2-difluoroethyl)-2,6-dimethylphenyl)-6-oxo-N-(5-((R)- |
| | piperidin-2-yl)pyridin-3-yl)-1,6-dihydropyrimidine-5-carboxamide |
| | Intermediate B40, Intermediate A4 |
| | HPLC-3 (10-40% MeCN); White solid (22.8 mg, 50%); LCMS m/z = 483 |
| | [M + H]⁺; ¹H NMR (400 MHZ, CDCl₃): 1.47-1.64 (m, 1H), 1.77 (q, 2H), |
| | 1.87-2.02 (m, 3H), 2.23 (d, 6H), 2.71-2.92 (m, 1H), 3.19 (d, 1H), 3.71-3.96 |
| | (m, 1H), 4.49-4.88 (m, 2H), 5.58-5.87 (m, 1H), 5.99 (d, 1H), 7.26 (s, 2H), |
| | 7.73 (s, 1H), 8.29-8.38 (m, 1H), 8.41 (s, 1H), 8.58 (d, 1H), 9.95 (br d, 1H), |
| | 11.92 (s, 1H). |
| 112 | 4-amino-N-(5-((R)-azepan-2-yl)pyridin-3-yl)-1-(4-((S)-1,2-difluoroethyl)- |
| | 2,6-dimethylphenyl)-6-oxo-1,6-dihydropyrimidine-5-carboxamide or 4- |
| | amino-N-(5-((R)-azepan-2-yl)pyridin-3-yl)-1-(4-((R)-1,2-difluoroethyl)-2,6- |
| | dimethylphenyl)-6-oxo-1,6-dihydropyrimidine-5-carboxamide or 4-amino- |
| | N-(5-((S)-azepan-2-yl)pyridin-3-yl)-1-(4-((S)-1,2-difluoroethyl)-2,6- |
| | dimethylphenyl)-6-oxo-1,6-dihydropyrimidine-5-carboxamide or 4-amino- |
| | N-(5-((S)-azepan-2-yl)pyridin-3-yl)-1-(4-((R)-1,2-difluoroethyl)-2,6- |
| | dimethylphenyl)-6-oxo-1,6-dihydropyrimidine-5-carboxamide |
| | Intermediate B40, Intermediate A29 |
| | HPLC-1 (10-50% MeCN); White solid (28.9 mg, 44%); LCMS m/z = 497 |
| | [M + H]⁺; ¹H NMR (400 MHZ, CDCl₃): 1.57-1.82 (m, 4H), 1.88 (d, 2H), 2.01 |
| | (br d, 2H), 2.21 (s, 6H), 2.85-3.21 (m, 2H), 3.98 (s, 1H), 4.48-4.82 (m, 2H), |
| | 5.61-5.79 (m, 1H), 6.19 (d, 1H), 7.24 (s, 2H), 7.71 (s, 1H), 8.26-8.35 (m, |
| | 2H), 8.60 (s, 1H), 9.94 (br s, 1H), 11.92 (br s, 1H). |
| 113 | 4-amino-1-(4-((R)-1,2-difluoroethyl)-2,6-dimethylphenyl)-6-oxo-N-(5-((R)- |
| | piperidin-2-yl)pyridin-3-yl)-1,6-dihydropyrimidine-5-carboxamide or 4- |
| | amino-1-(4-((S)-1,2-difluoroethyl)-2,6-dimethylphenyl)-6-oxo-N-(5-((R)- |
| | piperidin-2-yl)pyridin-3-yl)-1,6-dihydropyrimidine-5-carboxamide |
| | Intermediate B41, Intermediate A4 |
| | White solid (42 mg, 84%); LCMS m/z = 483 [M + H]⁺; ¹H NMR (400 MHZ, |
| | CDCl₃): 11.94 (s, 1H), 9.93 (d, 1H), 8.59 (s, 1H), 8.37 (d, 2H), 7.73 (s, 1H), |
| | 7.25 (s, 2H), 6.20 (d, 1H), 5.82-5.61 (m, 1H), 4.77-4.63 (m, 2H), 3.84 (dd, |
| | 1H), 3.19 (d, 1H), 2.81 (t, 1H), 2.22 (d, 6H), 2.00-1.84 (m, 3H), 1.79-1.68 |
| | (m, 2H), 1.62-1.48 (m, 1H). |
| 114 | 4-amino-N-(5-((R)-1-aminoethyl)pyridin-3-yl)-1-(4-((S)-1,2-difluoroethyl)- |
| | 2,6-dimethylphenyl)-6-oxo-1,6-dihydropyrimidine-5-carboxamide or 4- |
| | amino-N-(5-((R)-1-aminoethyl)pyridin-3-yl)-1-(4-((R)-1,2-difluoroethyl)- |
| | 2,6-dimethylphenyl)-6-oxo-1,6-dihydropyrimidine-5-carboxamide or 4- |
| | amino-N-(5-((S)-1-aminoethyl)pyridin-3-yl)-1-(4-((S)-1,2-difluoroethyl)- |
| | 2,6-dimethylphenyl)-6-oxo-1,6-dihydropyrimidine-5-carboxamide or 4- |
| | amino-N-(5-((S)-1-aminoethyl)pyridin-3-yl)-1-(4-((R)-1,2-difluoroethyl)- |
| | 2,6-dimethylphenyl)-6-oxo-1,6-dihydropyrimidine-5-carboxamide |
| | Intermediate B41, Intermediate A16 |
| | HPLC-3 (5-30% MeCN); White solid (11.1 mg, 31%); LCMS m/z = 443 |
| | [M + H]⁺; ¹H NMR (400 MHZ, CDCl₃): 11.98 (s, 1H), 9.91 (s, 1H), 8.60 (s, |
| | 1H), 8.33 (br s, 3H), 7.74 (s, 1H), 7.26 (s, 2H), 6.25 (s, 1H), 5.88-5.61 (m, |
| | 1H), 4.82-4.74 (m, 1H), 4.73-4.68 (m, 1H), 4.67-4.63 (m, 1H), 4.61-4.57 |
| | (m, 1H), 4.30 (d, 1H), 2.23 (d, 6H), 1.53 (d, 3H). |
| 115 | 4-amino-N-(5-((R)-azepan-2-yl)pyridin-3-yl)-1-(4-((S)-1,2-difluoroethyl)- |
| | 2,6-dimethylphenyl)-6-oxo-1,6-dihydropyrimidine-5-carboxamide or 4- |
| | amino-N-(5-((R)-azepan-2-yl)pyridin-3-yl)-1-(4-((R)-1,2-difluoroethyl)-2,6- |
| | dimethylphenyl)-6-oxo-1,6-dihydropyrimidine-5-carboxamide or 4-amino- |
| | N-(5-((S)-azepan-2-yl)pyridin-3-yl)-1-(4-((S)-1,2-difluoroethyl)-2,6- |
| | dimethylphenyl)-6-oxo-1,6-dihydropyrimidine-5-carboxamide or 4-amino- |
| | N-(5-((S)-azepan-2-yl)pyridin-3-yl)-1-(4-((R)-1,2-difluoroethyl)-2,6- |
| | dimethylphenyl)-6-oxo-1,6-dihydropyrimidine-5-carboxamide |
| | Intermediate B41, Intermediate A29 |
| | HPLC-1 (20-50% MeCN); White solid (22.1 mg, 41%); LCMS m/z = 497 |
| | [M + H]⁺; ¹H NMR (400 MHZ, CDCl₃): 11.92 (s, 1H), 9.98 (d, 1H), 8.65 (d, |
| | 1H), 8.34 (s, 1H), 8.26 (s, 1H), 7.73 (s, 1H), 7.29 (s, 2H), 6.04 (d, 1H), 5.87- |
| | 5.59 (m, 1H), 4.81-4.52 (m, 2H), 3.98 (dd, 1H), 3.15 (td, 1H), 3.04 - 2.85 |
| | (m, 1H), 2.23 (s, 6H), 2.11-1.55 (m, 8H). |
| 116 | (R)-4-amino-1-(2,6-dichloro-4-(1,2-difluoroethyl)phenyl)-N-(5- |
| | ((methylamino)methyl)pyridin-3-yl)-6-oxo-1,6-dihydropyrimidine-5- |
| | carboxamide or (S)-4-amino-1-(2,6-dichloro-4-(1,2-difluoroethyl)phenyl)- |
| | N-(5-((methylamino)methyl)pyridin-3-yl)-6-oxo-1,6-dihydropyrimidine-5- |
| | carboxamide |
| | Intermediate B42, Intermediate A56 |
| | HPLC-1 1-50% MeCN; White solid 40.8 mg, 88%); LCMS m/z = 483 |
| | [M + H]⁺; ¹H NMR (400 MHZ, CDCl₃): 11.72 (s, 1H), 9.98 (d, 1H), 8.57 (s, |
| | 1H), 8.42 (s, 1H), 8.31 (s, 1H), 7.71 (s, 1H), 7.56 (s, 2H), 6.38-6.38 (m, 1H), |
| | 5.82-5.61 (m, 1H), 4.83-4.55 (m, 2H), 3.96 (s, 2H), 2.52 (s, 3H). |
| 117 | (R)-4-amino-1-(2,6-dichloro-4-(1,2-difluoroethyl)phenyl)-N-(5- |
| | ((methylamino)methyl)pyridin-3-yl)-6-oxo-1,6-dihydropyrimidine-5- |
| | carboxamide or (S)-4-amino-1-(2,6-dichloro-4-(1,2-difluoroethyl)phenyl)- |
| | N-(5-((methylamino)methyl)pyridin-3-yl)-6-oxo-1,6-dihydropyrimidine-5- |
| | carboxamide |
| | Intermediate B43, Intermediate A56 |
| | HPLC-1 (5-35% MeCN); White solid (27.8 mg, 61%); LCMS m/z = 483 |
| | [M + H]⁺; ¹H NMR (400 MHZ, CDCl₃): 11.72 (s, 1H), 10.05 (br d, 1H), 8.62 |
| | (d, 1H), 8.34 (br d, 2H), 7.73 (s, 1H), 7.58 (s, 2H), 6.16 (br d, 1H), 5.84- |
| | 5.63 (m, 1H), 4.85-4.71 (m, 1H), 4.71-4.58 (m, 1H), 3.91 (s, 2H), 2.51 (s, |
| | 3H). |
| 118 | (S)-4-amino-1-(2,6-dichloro-4-(trifluoromethyl)phenyl)-6-oxo-N-(5- |
| | (pyrrolidin-2-yl)pyridin-3-yl)-1,6-dihydropyrimidine-5-carboxamide |
| | Intermediate B23, Intermediate A13 |
| | HPLC-1 (1-50% MeCN); White solid (22.1 mg, 50%); LCMS m/z = 514 |
| | [M + H]⁺; ¹H NMR (400 MHZ, DMSO-d₆): 1.53-1.66 (m, 1H), 1.74-1.88 (m, |
| | 2H), 2.13-2.24 (m, 1H), 2.94-3.00 (m, 1H), 3.03-3.08 (m, 1H), 4.17 (br.t, |
| | 2H), 8.04 (t, 1H), 8.24 (s, 1H), 8.26 (br. s, 1H), 8.28 (d, 1H), 8.32 (s, 2H), |
| | 8.48 (s, 1H), 8.70 (d, 1H), 8.85 (br.d, 1H), 9.50-9.85 (m, 1H), 11.55-11.93 |
| | (m, 1H). |
| 119 | (S)-4-amino-1-(2,6-dichloro-4-(trifluoromethyl)phenyl)-6-oxo-N-(5- |
| | (piperidin-2-yl)pyridin-3-yl)-1,6-dihydropyrimidine-5-carboxamide |
| | Intermediate B23, Intermediate A5 |
| | HPLC-1 (1-50% MeCN); White solid (14 mg, 31%); LCMS m/z = 527 |
| | [M + H]⁺; ¹H NMR (400 MHZ, DMSO-d₆): 1.37-1.50 (m, 3 H), 1.60 (d, 1H), |
| | 1.70-1.85 (m, 2 H), 2.64-2.75 (m, 1 H), 3.09 (d, 1H), 3.68 (d, 1H), 8.08 (t, |
| | 1H), 8.27 (d, 1H), 8.24 (d, 1H), 8.33 (s, 2H), 8.48 (s, 1H), 8.67 (d, 1H), 8.86 |
| | (d, 1H), 9.69 (d, 1H), 11.71 (s, 1H). |
| 120 | 4-amino-N-(5-(aminomethyl)pyridin-3-yl)-1-(2,6-dichloro-4- |
| | (trifluoromethyl)phenyl)-6-oxo-1,6-dihydropyrimidine-5-carboxamide |
| | Intermediate B23, Intermediate A50 |
| | HPLC-1 (1-30% MeCN); White solid (10.8 mg, 15%); LCMS m/z = 473 |
| | [M + H]⁺; ¹H NMR (400 MHZ, CDCl₃): 11.65 (s, 1H), 10.11 (d, 1H), 8.62 (d, |
| | 1H), 8.31 (s, 1H), 8.23 (s, 1H), 7.85 (s, 2H), 7.74 (s, 1H), 6.19 (d, 1H), 3.95 |
| | (s, 2H). |
| 121 | (R)-4-amino-1-(2,6-dichloro-4-(trifluoromethyl)phenyl)-N-(5-(2-methoxy- |
| | 1-(methylamino)ethyl)pyridin-3-yl)-6-oxo-1,6-dihydropyrimidine-5- |
| | carboxamide or (S)-4-amino-1-(2,6-dichloro-4-(trifluoromethyl)phenyl)-N- |
| | (5-(2-methoxy-1-(methylamino)ethyl)pyridin-3-yl)-6-oxo-1,6- |
| | dihydropyrimidine-5-carboxamide |
| | Intermediate B23, Intermediate A23 |
| | HPLC-1 (25-55% MeCN); White solid (36.3 mg, 56%); LCMS m/z = 531 |
| | [M + H]⁺; ¹H NMR (400 MHZ, CDCl₃): 2.62 (s, 3H), 3.39 (s, 3H), 3.76 (dd, |
| | 1H), 3.96 (br dd, 1H), 4.28 (br dd, 1H), 6.26 (br d, 1H), 7.72 (s, 1H), 7.82 |
| | (s, 2H), 8.41 (br s, 1H) 8.60 (s, 1H), 8.67 (br s, 1H), 10.03 (br d, 1H), 11.76 |
| | (s, 1H). |
| 122 | (R)-4-amino-1-(2,6-dichloro-4-(trifluoromethyl)phenyl)-N-(5-(2-methoxy- |
| | 1-(methylamino)ethyl)pyridin-3-yl)-6-oxo-1,6-dihydropyrimidine-5- |
| | carboxamide or (S)-4-amino-1-(2,6-dichloro-4-(trifluoromethyl)phenyl)-N- |
| | (5-(2-methoxy-1-(methylamino)ethyl)pyridin-3-yl)-6-oxo-1,6- |
| | dihydropyrimidine-5-carboxamide |
| | Intermediate B23, Intermediate A22 |
| | HPLC-3 (5-35% MeCN); White solid (24.4 mg, 53%); LCMS m/z = 517 |
| | [M + H]⁺; ¹H NMR (400 MHZ, CDCl₃): 11.64 (s, 1 H), 10.13 (br s, 1H), 8.71 |
| | (d, 1H), 8.35 (s, 1H), 8.22 (br s, 1H), 7.84 (s, 2H), 7.73 (s, 1H), 6.24 (br d, |
| | 1H), 4.26 (br d, 1H), 3.51-3.60 (m, 1H), 3.45-3.45 (m, 1H), 3.45 (br d, 1H), |
| | 3.41 (s, 3H). |
| 123 | (S)-4-amino-1-(2,6-dichloro-4-(dimethylamino)phenyl)-6-oxo-N-(5- |
| | (piperidin-2-yl)pyridin-3-yl)-1,6-dihydropyrimidine-5-carboxamide |
| | Intermediate B8, Intermediate A5 |
| | HPLC-1 (1-70% MeCN); Yellow solid (23.4 mg, 85%); LCMS m/z = 502 |
| | [M + H]⁺; ¹H NMR (400 MHZ, CDCl₃): 11.90 (s, 1H), 9.96 (br d, 1H), 8.59 |
| | (s, 1H), 8.39 (br d, 2H), 7.75 (s, 1H), 6.72 (s, 2H), 6.12 (br s, 1H), 3.85-3.80 |
| | (m, 1H), 3.14 (br d, 1H), 3.03 (s, 6H), 2.78 (dt, 1H), 1.98-1.92 (m, 1H), 1.89 |
| | (br t, 2H), 1.76 (br d, 1H), 1.74-1.63 (m, 2H), 1.55-1.48 (m, 1H). |
| 124 | (S)-4-amino-1-(2,6-dichloro-4-isopropoxyphenyl)-6-oxo-N-(5-(piperidin-2- |
| | yl)pyridin-3-yl)-1,6-dihydropyrimidine-5-carboxamide |
| | Intermediate B31, Intermediate A5 |
| | HPLC-2 (5-40% MeCN); White solid (34.1 mg, 75%); LCMS m/z = 517 |
| | [M + H]⁺; ¹H NMR (400 MHZ, DMSO-d₆): 11.91 (s, 1H), 9.63 (d, 1H), 8.78- |
| | 8.72 (m, 2H), 8.43 (s, 1H), 8.30 (s, 1H), 8.24 (s, 1H), 8.13 (s, 1H), 7.36 (s, |
| | 2H), 4.84 (td, 1H), 3.83 (d, 1H), 3.16 (d, 1H), 2.79 (t, 1H), 1.88-1.76 (m, |
| | 2H), 1.65 (s, 1H), 1.51 (d, 3H), 1.32 (d, 6H). |
| 125 | (R)-4-amino-1-(2,6-dimethyl-4-propoxyphenyl)-6-oxo-N-(5-(piperidin-2- |
| | yl)pyridin-3-yl)-1,6-dihydropyrimidine-5-carboxamide |
| | Intermediate B26, Intermediate A4 |
| | HPLC-1 (5-40% MeCN); Yellow solid (26 mg, 57%); LCMS m/z = 477 |
| | [M + H]⁺; ¹H NMR (400 MHZ, CDCl₃): 12.06 (s, 1H), 9.94 (br.d, 1H), 8.68 |
| | (d, 1H), 8.36 (d, 1H), 8.24 (t, 1H), 7.77 (s, 1H), 6.76 (s, 2H), 5.95 (br.d, |
| | 1H), 3.96 (t, 2H), 3.72 (dd, 1H), 3.21 (br.d, 1H), 2.80 (td, 1H), 2.15 (s, 6H), |
| | 1.93 (br.d, 1H), 1.87-1.83 (m, 2H), 1.67 (br.d, 2H), 1.60-1.47 (m, 1H), 1.07 |
| | (t, 3H). |
| 126 | (R)-4-amino-N-(5-(1-aminoethyl)pyridin-3-yl)-1-(2,6-dimethyl-4- |
| | propoxyphenyl)-6-oxo-1,6-dihydropyrimidine-5-carboxamide or (S)-4- |
| | amino-N-(5-(1-aminoethyl)pyridin-3-yl)-1-(2,6-dimethyl-4- |
| | propoxyphenyl)-6-oxo-1,6-dihydropyrimidine-5-carboxamide |
| | Intermediate B26, Intermediate A16 |
| | HPLC-1 (5-35% MeCN); White solid (18.5 mg, 76%); LCMS m/z = 437 |
| | [M + H]⁺; ¹H NMR (400 MHz, CDCl₃): 0.97 (t, 3H), 1.39 (d, 3H), 1.64-1.82 |
| | (m, 2 H), 2.05 (d, 6H), 3.86 (t, 2H), 4.14 (d, 1H), 5.86-6.17 (m, 1H), 6.66 (s, |
| | 2H), 7.67 (s, 1H), 8.20 (d, 2H), 8.53 (s, 1H), 9.79 (d, 1H), 12.00 (s, 1H). |
| 127 | 4-amino-1-(2,6-dimethyl-4-propoxyphenyl)-N-(5- |
| | ((methylamino)methyl)pyridin-3-yl)-6-oxo-1,6-dihydropyrimidine-5- |
| | carboxamide |
| | Intermediate B26, Intermediate A56 |
| | HPLC-1 (1-40% MeCN); White solid (39 mg, 62%); LCMS m/z = 437 |
| | [M + H]⁺; ¹H NMR (400 MHZ, CDCl₃): 12.11 (s, 1H), 9.88 (br.d, 1H), 8.58 |
| | (s, 1H), 8.39 (br.s, 1H), 8.31 (s, 1H), 7.75 (s, 1H), 6.73 (s, 2H), 6.12 (br.d, |
| | 1H), 3.97-3.87 (m, 4H), 2.48 (s,3H), 2.13 (s, 6H), 1.86-1.76 (m, 2H), 1.05 |
| | (br.t, 3H). |
| 128 | (R)-4-amino-N-(5-(1-aminopropyl)pyridin-3-yl)-1-(4-(difluoromethyl)-2,6- |
| | dimethylphenyl)-6-oxo-1,6-dihydropyrimidine-5-carboxamide or (S)-4- |
| | amino-N-(5-(1-aminopropyl)pyridin-3-yl)-1-(4-(difluoromethyl)-2,6- |
| | dimethylphenyl)-6-oxo-1,6-dihydropyrimidine-5-carboxamide |
| | Intermediate B17, Intermediate A17 |
| | HPLC-1 (1-30% MeCN); White solid (23.7 mg, 47%); LCMS m/z = 443 |
| | [M + H]⁺; ¹H NMR (400 MHZ, CDCl₃): 0.66-0.86 (m, 3H), 1.64-1.93 (m, |
| | 2H), 2.14 (d, 6H), 3.91 (s, 1H), 6.35 (s, 1H), 6.57 (t, 1H), 7.30 (s, 2H), 7.63 |
| | (s, 1H), 8.17 (s, 1H), 8.24 (s, 1H), 8.45 (s, 1H), 9.76 (d, 1H), 11.85 (s, 1H). |
| 129 | (R)-4-amino-N-(5-(1-aminopropyl)pyridin-3-yl)-1-(2,6-dichloro-4- |
| | (ethoxymethyl)phenyl)-6-oxo-1,6-dihydropyrimidine-5-carboxamide or (S)- |
| | 4-amino-N-(5-(1-aminopropyl)pyridin-3-yl)-1-(2,6-dichloro-4- |
| | (ethoxymethyl)phenyl)-6-oxo-1,6-dihydropyrimidine-5-carboxamide |
| | Intermediate B28, Intermediate A8 |
| | HPLC-1 (1-40% MeCN); White solid (11.3 mg, 30%); LCMS m/z = 492 |
| | [M + H]⁺; ¹H NMR (400 MHZ, CDCl₃): 0.83 (t, 3H), 1.29 (t, 3H), 1.76-1.92 |
| | (m, 2H), 3.61 (q, 2H), 3.94 (t, 1H), 4.53 (s, 2H), 6.49 (s, 1H), 7.51 (s, 2H), |
| | 7.70 (s, 1 H), 8.26 (d, 2H), 8.57 (d, 1H), 9.95 (d, 1H), 11.76 (s, 1 H). |
| 130 | (R)-4-amino-N-(5-(1-aminoethyl)pyridin-3-yl)-1-(4-(ethoxymethyl)-2,6- |
| | dimethylphenyl)-6-oxo-1,6-dihydropyrimidine-5-carboxamide or (S)-4- |
| | amino-N-(5-(1-aminoethyl)pyridin-3-yl)-1-(4-(ethoxymethyl)-2,6- |
| | dimethylphenyl)-6-oxo-1,6-dihydropyrimidine-5-carboxamide |
| | Intermediate B29, Intermediate A16 |
| | HPLC-1 (10-30% MeCN); White solid (17.2 mg, 47%); LCMS m/z = 437 |
| | [M + H]⁺; ¹H NMR (400 MHZ, CDCl₃): 11.94 (s, 1H), 9.73 (d, 1H), 8.49 (s, |
| | 1H), 8.50-8.14 (m, 2H), 7.63 (s, 1H), 7.13 (s, 2H), 6.11 (d, 1H), 4.42 (s, |
| | 3H), 4.42-4.16 (m, 1H), 3.52 (q, 2H), 2.09 (d, 6H), 1.43 (s, 3H), 1.20 (t, |
| | 3H). |
| 131 | (R)-4-amino-1-(4-(ethoxymethyl)-2,6-dimethylphenyl)-6-oxo-N-(5- |
| | (piperidin-2-yl)pyridin-3-yl)-1,6-dihydropyrimidine-5-carboxamide |
| | Intermediate B29, Intermediate A4 |
| | HPLC-3 (1-30% MeCN); White solid (50.9 mg, 80%); LCMS m/z = 477 |
| | [M + H]⁺; ¹H NMR (400 MHZ, CDCl₃): 1.28 (t, 3H), 1.46-1.59 (m, 1H), |
| | 1.63-1.80 (m, 2H), 1.83-1.98 (m, 3H), 2.17 (s, 6H), 2.77 (td, 1H), 3.14 (d, |
| | 1H), 3.60 (q, 2H), 3.76-3.86 (m, 1H), 4.50 (s, 2H), 6.21 (d, 1H), 7.21 (s, 2 |
| | H), 7.71 (s, 1H), 8.32-8.40 (m, 2H), 8.58 (d, 1H), 9.90 (d, 1H), 12.02 (s, |
| | 1H). |
| 132 | (R)-4-amino-N-(5-(azepan-2-yl)pyridin-3-yl)-1-(4-(ethoxymethyl)-2,6- |
| | dimethylphenyl)-6-oxo-1,6-dihydropyrimidine-5-carboxamide or (S)-4- |
| | amino-N-(5-(azepan-2-yl)pyridin-3-yl)-1-(4-(ethoxymethyl)-2,6- |
| | dimethylphenyl)-6-oxo-1,6-dihydropyrimidine-5-carboxamide |
| | Intermediate B29, Intermediate A30 |
| | HPLC-1 (15-45% MeCN); White solid (10.7 mg, 39%); LCMS m/z = 490 |
| | [M + H]⁺; ¹H NMR (400 MHZ, CDCl₃): 11.93 (s, 1H), 9.84 (d, 1H), 8.53 (d, |
| | 1H), 8.26 (s, 1H), 8.21 (s, 1H), 7.64 (s, 1H), 7.14 (s, 2H), 5.98 (d, 1H), 4.43 |
| | (s, 2H), 3.91 (t, 1H), 3.52 (q, 2H), 3.14-2.96 (m, 1H), 2.92-2.79 (m, 1H), |
| | 2.10 (s, 6H), 1.99-1.90 (m, 2H), 1.88-1.76 (m, 2H), 1.75-1.47 (m, 4H), 1.21 |
| | (t, 3H). |
| 133 | (S)-4-amino-1-(2,6-dichloro-4-ethoxyphenyl)-6-oxo-N-(5-(piperidin-2- |
| | yl)pyridin-3-yl)-1,6-dihydropyrimidine-5-carboxamide |
| | Intermediate B30, Intermediate A5 |
| | HPLC-1 (5-45% MeCN); White solid (10.9 mg, 30%); LCMS m/z = 503 |
| | [M + H]⁺; ¹H NMR (400 MHZ, CDCl₃): 11.80 (s, 1H), 10.00 (d, 1H), 8.63 (s, |
| | 1H), 8.37 (br.s, 1H), 8.31 (s, 1H), 7.72 (s, 1H), 7.04 (s, 2H), 6.15 (d, 1H), |
| | 4.09 (q, 2H), 3.79-3.74 (m, 1H), 3.19 (d, 2H), 2.83-2.75 (m, 1H), 2.03-1.87 |
| | (m, 2H), 1.87-1.82 (m, 1H), 1.80-1.75 (m, 1H), 1.73-1.68 (m, 2H), 1.46 (t, |
| | 3H). |
| 134 | (R)-4-amino-N-(5-(1-amino-2-methoxyethyl)pyridin-3-yl)-1-(2,6-dichloro- |
| | 4-ethoxyphenyl)-6-oxo-1,6-dihydropyrimidine-5-carboxamide or (S)-4- |
| | amino-N-(5-(1-amino-2-methoxyethyl)pyridin-3-yl)-1-(2,6-dichloro-4- |
| | ethoxyphenyl)-6-oxo-1,6-dihydropyrimidine-5-carboxamide |
| | Intermediate B30, Intermediate A22 |
| | HPLC-1 (15-45% MeCN); White solid (10.7 mg, 39%); LCMS m/z = 490 |
| | [M + H]⁺; ¹H NMR (400 MHz, CDCl₃): 11.80 (s, 1H), 10.00 (d, 1H), 8.63 (s, |
| | 1H), 8.37 (br.s, 1H), 8.31 (s,1H), 7.72 (s, 1H), 7.04 (s, 2H), 6.15 (d, 1H), |
| | 4.09 (q, 2H), 3.79-3.74 (m, 1H), 3.19 (d, 2H), 2.83-2.75 (m, 1H), 2.03-1.87 |
| | (m, 2H), 1.87-1.82 (m, 1H), 1.80-1.75 (m, 1H), 1.73-1.68 (m, 2H), 1.46 (t, |
| | 3H). |
| 135 | (R)-N-(5-(1,4-oxazepan-3-yl)pyridin-3-yl)-4-amino-1-(2,6-dichloro-4- |
| | ethoxyphenyl)-6-oxo-1,6-dihydropyrimidine-5-carboxamide or (S)-N-(5- |
| | (1,4-oxazepan-3-yl)pyridin-3-yl)-4-amino-1-(2,6-dichloro-4-ethoxyphenyl)- |
| | 6-oxo-1,6-dihydropyrimidine-5-carboxamide |
| | Intermediate B30, Intermediate A27 |
| | HPLC-1 (1-40% MeCN); White solid (20.3 mg, 44%); LCMS m/z = 519 |
| | [M + H]⁺; ¹H NMR (400 MHZ, CDCl₃): 11.81 (s, 1H), 10.00 (d, 1H), 8.70 (d, |
| | 1H), 8.31 (d, 1H), 8.21 (s, 1H), 7.73 (s, 1H), 7.05 (s, 2H), 6.12 (d, 1H), |
| | 4.12-4.03 (m, 3H), 4.01-3.91 (m, 2H), 3.89-3.81 (m, 1H), 3.62 (dd, 1H), |
| | 3.22 (td, 1H), 3.04 (ddd, 1H), 2.12-1.94 (m, 2H), 1.46 (t, 3H). |
| 136 | (R)-4-amino-N-(5-(1-aminopropyl)pyridin-3-yl)-1-(2,6-dichloro-4- |
| | ethoxyphenyl)-6-oxo-1,6-dihydropyrimidine-5-carboxamide or (S)-4- |
| | amino-N-(5-(1-aminopropyl)pyridin-3-yl)-1-(2,6-dichloro-4-ethoxyphenyl)- |
| | 6-oxo-1,6-dihydropyrimidine-5-carboxamide |
| | Intermediate B30, Intermediate A8 |
| | HPLC-3 (15-35% MeCN); White solid (33.7 mg, 92%); LCMS m/z = 477 |
| | [M + H]⁺; ¹H NMR (400 MHZ, CDCl₃): 11.78 (s, 1H), 9.86 (d, 1H), 8.51 (s, |
| | 1H), 8.28-8.17 (m, 2H), 7.68 (s, 1H), 7.00 (s, 2H), 6.46 (d, 1H), 4.04 (q, |
| | 2H), 3.99-3.91 (m, 1H), 1.96-1.68 (m, 2H), 1.42 (t, 3H, J = 6.8 Hz), 0.80 (t, |
| | 3H). |
| 137 | (R)-4-amino-N-(5-(1-aminoethyl)pyridin-3-yl)-1-(2,6-dichloro-4- |
| | ethoxyphenyl)-6-oxo-1,6-dihydropyrimidine-5-carboxamide or (S)-4- |
| | amino-N-(5-(1-aminoethyl)pyridin-3-yl)-1-(2,6-dichloro-4-ethoxyphenyl)- |
| | 6-oxo-1,6-dihydropyrimidine-5-carboxamide |
| | Intermediate B30, Intermediate A16 |
| | HPLC-1 (10-50% MeCN); White solid (21.9 mg, 65%); LCMS m/z = 463 |
| | [M + H]⁺; ¹H NMR (400 MHZ, CDCl₃): 11.87 (s, 1H), 9.63 (s, 1H), 8.77 (s, |
| | 1H), 8.72 (s, 1H), 8.41 (s, 1H), 8.30 (s, 1H), 8.04 (s, 1H), 7.36 (s, 2H), 4.21- |
| | 4.15 (m, 3H), 1.38-1.33 (m, 6H). |
| 138 | 4-amino-1-(2,6-dichloro-4-ethoxyphenyl)-N-(5-(((ethyl- |
| | d5)amino)methyl)pyridin-3-yl)-6-oxo-1,6-dihydropyrimidine-5- |
| | carboxamide |
| | Intermediate B30, Intermediate A53 |
| | HPLC-1 (1-40% MeCN); White solid (32.1 mg, 88%); LCMS m/z = 482 |
| | [M + H]⁺; ¹H NMR (400 MHZ, CDCl₃): 11.84 (s, 1H), 10.00 (br d, 1H), 8.60 |
| | (d, 1H), 8.43 (br s, 1H), 8.34 (s, 1H), 7.74 (s, 1H), 7.06 (s, 2H), 6.07 (d, |
| | 1H), 4.10 (q, 2H), 3.96 (s, 2H), 1.48 (t, 3H). |
| 139 | (R)-4-amino-N-(5-(amino(cyclopropyl)methyl)pyridin-3-yl)-1-(2,6- |
| | dichloro-4-ethoxyphenyl)-6-oxo-1,6-dihydropyrimidine-5-carboxamide or |
| | (S)-4-amino-N-(5-(amino(cyclopropyl)methyl)pyridin-3-yl)-1-(2,6- |
| | dichloro-4-ethoxyphenyl)-6-oxo-1,6-dihydropyrimidine-5-carboxamide |
| | Intermediate B30, Intermediate A9 |
| | HPLC-1 (15-45% MeCN); Yellow solid (36.4 mg, 50%); LCMS m/z = 489 |
| | [M + H]⁺; ¹H NMR (400 MHZ, CDCl₃): 11.81 (s, 1H), 9.82 (br s, 1H), 8.51 |
| | (s, 1H), 8.39 (br s, 1H), 8.31 (br s, 1H), 7.71 (s, 1H), 7.02 (s, 2H), 6.53 (br s, |
| | 1H), 4.07 (q, 2H), 3.41 (br d, 1H), 1.45 (t, 3H), 1.30 (br d, 1H), 0.68 (br s, |
| | 1H), 0.57-0.48 (m, 2H), 0.27 (br d, 1H). |
| 140 | (R)-4-amino-N-(5-(1-amino-2-ethoxyethyl)pyridin-3-yl)-1-(2,6-dichloro-4- |
| | ethoxyphenyl)-6-oxo-1,6-dihydropyrimidine-5-carboxamide or (S)-4- |
| | amino-N-(5-(1-amino-2-ethoxyethyl)pyridin-3-yl)-1-(2,6-dichloro-4- |
| | ethoxyphenyl)-6-oxo-1,6-dihydropyrimidine-5-carboxamide |
| | Intermediate B30, Intermediate A10 |
| | HPLC-1 (10-40% MeCN); White solid (32.4 mg, 97%); LCMS m/z = 507 |
| | [M + H]⁺; ¹H NMR (400 MHZ, CDCl₃): 11.84 (s, 1H), 9.95 (d, 1H), 8.66 (d, |
| | 1H), 8.34 (d, 2H), 7.74 (s, 1H), 7.06 (s, 2H), 6.17 (s, 1H), 4.32 (dd, 1H), |
| | 4.10 (q, 2H), 3.63-3.66 (m, 1H), 3.56 (dd, 3H), 1.48 (t, 3H), 1.21 (t, 3H). |
| 141 | (R)-4-amino-1-(2,6-dichloro-4-ethoxyphenyl)-N-(5-(2-methoxy-1- |
| | (methylamino)ethyl)pyridin-3-yl)-6-oxo-1,6-dihydropyrimidine-5- |
| | carboxamide or (S)-4-amino-1-(2,6-dichloro-4-ethoxyphenyl)-N-(5-(2- |
| | methoxy-1-(methylamino)ethyl)pyridin-3-yl)-6-oxo-1,6-dihydropyrimidine- |
| | 5-carboxamide |
| | Intermediate B30, Intermediate A23 |
| | HPLC-1 (25-65% MeCN); White solid (29.6 mg, 59%); LCMS m/z = 507 |
| | [M + H]⁺; ¹H NMR (400 MHZ, CDCl₃): 12.28 (s, 1H), 10.13 (d, 1H), 8.92- |
| | 9.16 (m, 1H), 8.86 (s, 1H), 8.52-8.79 (m, 1H), 7.96 (s, 1H), 7.50 (s, 2H), |
| | 6.36-6.46 (m, 1H), 4.54 (s, 1H), 4.31 (q, 2H), 4.12-4.22 (m, 1H), 3.98-4.06 |
| | (m, 1H), 3.61 (s, 3H), 2.86 (s, 3H), 1.69 (t, 1H). |
| 142 | (R)-4-amino-N-(5-(1-aminoethyl)pyridin-3-yl)-1-(2,6-dichloro-4- |
| | ethoxyphenyl)-6-oxo-1,6-dihydropyrimidine-5-carboxamide or (S)-4- |
| | amino-N-(5-(1-aminoethyl)pyridin-3-yl)-1-(2,6-dichloro-4-ethoxyphenyl)- |
| | 6-oxo-1,6-dihydropyrimidine-5-carboxamide |
| | Intermediate B30, Intermediate A17 |
| | HPLC-1 (10-40% MeCN); White solid (32.2 mg, 68%); LCMS m/z = 464 |
| | [M + H]⁺; ¹H NMR (400 MHZ, CDCl₃): 11.81 (s, 1H), 9.96 (br s, 1H), 8.62 |
| | (s, 1H), 8.32-8.26 (m, 2H), 7.74 (s, 1H), 7.06 (s, 2H), 6.23-6.15 (m, 1H), |
| | 4.27-4.12 (m, 1H), 4.10 (q, 2H), 3.62 -3.44 (m, 3H), 1.50-1.46 (m, 6H). |
| 143 | (R)-4-amino-N-(5-(1-amino-3,3-difluoropropyl)pyridin-3-yl)-1-(2,6- |
| | dichloro-4-ethoxyphenyl)-6-oxo-1,6-dihydropyrimidine-5-carboxamide or |
| | (S)-4-amino-N-(5-(1-amino-3,3-difluoropropyl)pyridin-3-yl)-1-(2,6- |
| | dichloro-4-ethoxyphenyl)-6-oxo-1,6-dihydropyrimidine-5-carboxamide |
| | Intermediate B30, Intermediate A45 |
| | HPLC-1 (10-40% MeCN); White solid (42.1 mg, 77%); LCMS m/z = 513 |
| | [M + H]⁺; ¹H NMR (400 MHZ, CDCl₃): 11.86 (s, 1H), 10.02 (d, 1H), 8.70 (d, |
| | 1H), 8.31 (s, 1H), 8.23 (s, 1H), 7.75 (s, 1H), 7.07 (s, 2H), 6.09-5.76 (m, 2H), |
| | 4.25 (dd, 1H), 4.11 (q, 2H), 2.31-2.17 (m, 2H), 1.48 (t, 3H). |
| 144 | (R)-4-amino-N-(5-(1-aminoethyl)pyridin-3-yl)-1-(4-(2,2-difluoroethyl)-2,6- |
| | dimethylphenyl)-6-oxo-1,6-dihydropyrimidine-5-carboxamide or (S)-4- |
| | amino-N-(5-(1-aminoethyl)pyridin-3-yl)-1-(4-(2,2-difluoroethyl)-2,6- |
| | dimethylphenyl)-6-oxo-1,6-dihydropyrimidine-5-carboxamide |
| | Intermediate B32, Intermediate A16 |
| | HPLC-1 (10-40% MeCN); White solid (22.2 mg, 81%); LCMS m/z = 443 |
| | [M + H]⁺; ¹H NMR (400 MHZ, CDCl₃): 11.90 (s, 1H), 9.84 (d, 1H), 8.54 (s, |
| | 1H), 8.29-8.06 (m, 2H), 7.66 (s, 1H), 7.06 (s, 2H), 6.08-5.71 (m, 2H), 4.14 |
| | (s, 1H), 3.83-3.74 (m, 2H), 3.07 (td, 2H), 2.10 (d, 6H), 1.38 (s, 3H). |
| 145 | (R)-4-amino-1-mesityl-6-oxo-N-(5-(piperidin-2-yl)pyridin-3-yl)-1,6- |
| | dihydropyrimidine-5-carboxamide |
| | Intermediate B35, Intermediate A4 |
| | HPLC-3 (15-40% MeCN); White solid (23.9 mg, 47%); LCMS m/z = 433 |
| | [M + H]⁺; ¹H NMR (400 MHZ, CDCl₃): 12.06 (s, 1H), 9.89 (br.d, 1H), 8.59 |
| | (br.s, 1H), 8.48 (s, 1H), 8.30 (s, 1H), 7.77 (s, 1H), 7.07 (s, 2H), 5.98 (d, 1H), |
| | 3.96 (d, 1H), 3.16 (d, 1H), 2.93-2.77 (m, 1H), 2.38 (s, 3H), 2.17 (d, 6H), |
| | 2.09-1.92 (m, 3H), 1.91-1.71 (m, 2H), 1.68-1.49 (m, 1H). |
| 146 | (R)-4-amino-N-(5-(azepan-2-yl)pyridin-3-yl)-1-mesityl-6-oxo-1,6- |
| | dihydropyrimidine-5-carboxamide or (S)-4-amino-N-(5-(azepan-2- |
| | yl)pyridin-3-yl)-1-mesityl-6-oxo-1,6-dihydropyrimidine-5-carboxamide |
| | Intermediate B35, Intermediate A29 |
| | HPLC-1 (25-65% MeCN); White solid (31.3 mg, 63%); LCMS m/z = 447 |
| | [M + H]⁺; ¹H NMR (400 MHZ, CDCl₃): 12.06 (s, 1H), 9.73 (br.d, 1H), 8.47 |
| | (br.s, 1H), 8.43 (s, 1H), 8.24 (br.s, 1H), 7.66 (s, 1H), 6.96 (s, 2H), 5.95 |
| | (br.d, 1H), 4.08 (br.d, 1H), 3.10-2.89 (m, 2H), 2.28 (s, 3H), 2.25-2.15 |
| | (m,1H), 2.05 (d, 6H), 1.98-1.83 (m, 3H), 1.79-1.50 (m, 4H). |
| 147 | (R)-4-amino-N-(5-(1-aminoethyl)pyridin-3-yl)-1-mesityl-6-oxo-1,6- |
| | dihydropyrimidine-5-carboxamide or (S)-4-amino-N-(5-(1- |
| | aminoethyl)pyridin-3-yl)-1-mesityl-6-oxo-1,6-dihydropyrimidine-5- |
| | carboxamide |
| | Intermediate B35, Intermediate A16 |
| | HPLC-1 (1-35% MeCN); White solid (37.9 mg, 70%); LCMS m/z = 393 |
| | [M + H]⁺; ¹H NMR (400 MHZ, CDCl₃): 11.99 (s, 1H), 9.73 (d, 1H), 8.46 (d, |
| | 1H), 8.46-8.20 (m, 3H), 7.65 (s, 1H), 6.95 (s, 2H), 6.43-6.41 (m, 1H), 6.31- |
| | 6.26 (m, 1H), 6.39-6.17 (m, 1H), 4.19 (d, 1H), 2.26 (s, 3H), 2.04 (d, 6H,), |
| | 1.43 (d, 3H). |
| 148 | (R)-4-amino-N-(5-(azepan-2-yl)pyridin-3-yl)-1-(4-ethyl-2,6- |
| | dimethylphenyl)-6-oxo-1,6-dihydropyrimidine-5-carboxamide or (S)-4- |
| | amino-N-(5-(azepan-2-yl)pyridin-3-yl)-1-(4-ethyl-2,6-dimethylphenyl)-6- |
| | oxo-1,6-dihydropyrimidine-5-carboxamide |
| | Intermediate B58, Intermediate A29 |
| | HPLC-1 (15-45% MeCN); Pink solid (38.3 mg, 100%); LCMS m/z = 461 |
| | [M + H]⁺; ¹H NMR (400 MHZ, CDCl₃): 1.19 (t, 3H) 1.50-1.63 (m, 2H) 1.64- |
| | 1.74 (m, 2H) 1.75-1.88 (m, 2H) 1.89-1.98 (m, 2H) 2.07 (s, 6H) 2.57 (q, 2H) |
| | 2.77-2.92 (m, 1H) 3.03 (d, 1H) 3.89 (t, 1H) 5.98 (d, 1H) 6.98 (s, 2H) 7.19 |
| | (s, 1H) 7.67 (s, 1H) 8.16-8.27 (m, 1H) 8.19-8.27 (m, 1H) 8.53 (d, 1H) 9.84 |
| | (d, 1H) 11.96 (s, 1H). |
| 149 | (S)-4-amino-1-(2,6-dichloro-4-fluorophenyl)-6-oxo-N-(5-(pyrrolidin-2- |
| | yl)pyridin-3-yl)-1,6-dihydropyrimidine-5-carboxamide |
| | Intermediate B39, Intermediate A13 |
| | HPLC-2 (5-35% MeCN); White solid (19.9 mg, 54%); LCMS m/z = 463 |
| | [M + H]⁺; ¹H NMR (400 MHz, CDCl₃): 11.73 (s, 1H), 9.92 (br d, 1H), 8.62 |
| | (d, 1H), 8.41 (s, 2H), 7.70 (s, 1H), 7.31 (d, 2H), 6.25 (br d, 1H), 4.64-4.52 |
| | (m, 1H), 3.51-3.25 (m, 2H), 2.41 (dt, 1H), 2.28-2.17 (m, 2H), 2.16-2.02 (m, |
| | 1H). |
| 150 | (R)-4-amino-N-(5-(1-aminobut-3-yn-1-yl)pyridin-3-yl)-1-(2,6-dichloro-4- |
| | ethoxyphenyl)-6-oxo-1,6-dihydropyrimidine-5-carboxamide or (S)-4- |
| | amino-N-(5-(1-aminobut-3-yn-1-yl)pyridin-3-yl)-1-(2,6-dichloro-4- |
| | ethoxyphenyl)-6-oxo-1,6-dihydropyrimidine-5-carboxamide |
| | Intermediate B30, Intermediate A57B |
| | HPLC-1 (10-50% MeCN); White solid (11.8 mg, 27%); LCMS m/z = 487 |
| | [M + H]⁺; ¹H NMR (400 MHZ, CDCl₃): 11.86 (s, 1H), 10.04 (d, 1H), 8.72 (s, |
| | 1H), 8.37 (s, 1H), 8.28 (s, 1H), 7.77 (s, 1H), 7.08 (s, 2H), 6.18-6.09 (m, 1H), |
| | 4.26 (t, 1H), 4.12 (q, 2H), 2.69-2.53 (m, 2H), 2.10 (s, 1H), 1.50 (t, 3H). |
| 151 | (R)-4-amino-N-(5-(1-aminobut-3-yn-1-yl)pyridin-3-yl)-1-(2,6-dichloro-4- |
| | ethoxyphenyl)-6-oxo-1,6-dihydropyrimidine-5-carboxamide or (S)-4- |
| | amino-N-(5-(1-aminobut-3-yn-1-yl)pyridin-3-yl)-1-(2,6-dichloro-4- |
| | ethoxyphenyl)-6-oxo-1,6-dihydropyrimidine-5-carboxamide |
| | Intermediate B30, Intermediate A57A |
| | HPLC-1 (1-40% MeCN); White solid (21.8 mg, 35%); LCMS m/z = 487 |
| | [M + H]⁺; ¹H NMR (400 MHZ, CDCl₃): 11.84 (s, 1H), 10.06 (d, 1H), 8.72 (d, |
| | 1H), 8.36 (s, 1H), 8.25 (s, 1H), 7.76 (s, 1H), 7.07 (s, 2H), 6.11 (d, 1H), 4.23 |
| | (t, 1H), 4.11 (q, 2H), 2.66-2.52 (m, 2H), 2.08 (s, 1H), 1.49 (t, 3H). |
| 152 | (R)-4-amino-1-(3,5-dichloropyridin-4-yl)-6-oxo-N-(5-(piperidin-2- |
| | yl)pyridin-3-yl)-1,6-dihydropyrimidine-5-carboxamide |
| | Intermediate B52, Intermediate A4 |
| | HPLC-2 (5-35% MeCN); White solid (46.6 mg, 43%); LCMS m/z = 460 |
| | [M + H]⁺; ¹H NMR (400 MHZ, CDCl₃): 11.60 (s, 1H), 9.71 (d, 1H), 8.98 (s, |
| | 2H), 8.88 (d, 1H), 8.63 (d, 1H), 8.47 (s, 1H), 8.24 (d, 1H), 8.05 (s, 1H), |
| | 3.60-3.52 (m, 1H), 3.02 (d, 1H), 2.68-2.58 (m, 1H), 1.78 (d, 1H), 1.69 (d, |
| | 1H), 1.55 (d, 1H), 1.49-1.35 (m, 2H), 1.34-1.22 (m, 1H). |
| 153 | (R)-4-amino-N-(5-(1-aminopropyl)pyridin-3-yl)-1-(3,5-dichloropyridin-4- |
| | yl)-6-oxo-1,6-dihydropyrimidine-5-carboxamide or (S)-4-amino-N-(5-(1- |
| | aminopropyl)pyridin-3-yl)-1-(3,5-dichloropyridin-4-yl)-6-oxo-1,6- |
| | dihydropyrimidine-5-carboxamide |
| | Intermediate B52, Intermediate A60 |
| | HPLC-15 (20-40% MeCN); White solid (12.5 mg, 30%); LCMS m/z = 434 |
| | [M + H]⁺; ¹H NMR (400 MHZ, DMSO-d₆): 11.59 (s, 1H), 9.72 (br d, 1H), |
| | 8.99 (s, 2H), 8.88 (br d, 1H), 8.71 (d, 1H), 8.48 (s, 1H), 8.23 (d, 1H), 7.95 (t, |
| | 1H), 3.72 (t, 1H), 1.94 (br s, 2H), 1.65-1.50 (m, 2H), 0.78 (t, 3H). |
| 154 | 4-amino-1-(2,6-dichloro-4-(2-methoxyethyl)phenyl)-N-(5- |
| | ((ethylamino)methyl)pyridin-3-yl)-6-oxo-1,6-dihydropyrimidine-5- |
| | carboxamide |
| | Intermediate B49, Intermediate A24 |
| | HPLC-1 (1-40% MeCN); White solid (28.3 mg, 76%); LCMS m/z = 483 |
| | [M + H]⁺; ¹H NMR (400 MHZ, CDCl₃): 11.71 (s, 1H), 9.91 (br d, 1H), 8.50 |
| | (d, 1H), 8.36 (s, 1H), 8.25 (s, 1H), 7.63 (s, 1H), 7.34 (s, 2H), 5.98 (br d, |
| | 1H), 3.89 (s, 2H), 3.58 (t, 2H), 3.30 (s, 3H), 2.84 (t, 2H), 2.79-2.70 (m, 2H), |
| | 1.12 (t, 3H). |
| 155 | (R)-4-amino-N-(5-(1-aminoethyl)pyridin-3-yl)-1-(4-(2-fluoroethyl)-2,6- |
| | dimethylphenyl)-6-oxo-1,6-dihydropyrimidine-5-carboxamide or (S)-4- |
| | amino-N-(5-(1-aminoethyl)pyridin-3-yl)-1-(4-(2-fluoroethyl)-2,6- |
| | dimethylphenyl)-6-oxo-1,6-dihydropyrimidine-5-carboxamide |
| | Intermediate B33, Intermediate A16 |
| | HPLC-7 (1-25% MeCN); White solid (18.5 mg, 40%); LCMS m/z = 425 |
| | [M + H]⁺; ¹H NMR (400 MHZ, CDCl₃): 12.04-12.00 (m, 1H), 9.83 (br s, 1H), |
| | 8.56 (br s, 1H), 8.36-8.31 (m, 2H), 7.76 (d, 1H), 7.31 (d, 1H), 7.31-7.30 (m, |
| | 1H), 6.36 (br d, 1H), 6.09-5.73(m, 2H), 4.77-4.63 (m, 2H), 4.36-4.24 (m, |
| | 1H), 3.08-3.29 (m, 2H), 2.18 (d, 6H), 1.54-1.49 (m, 3H). |
| 156 | 4-amino-1-(4-(2,2-difluoroethyl)-2,6-dimethylphenyl)-N-(5- |
| | ((ethylamino)methyl)pyridin-3-yl)-6-oxo-1,6-dihydropyrimidine-5- |
| | carboxamide |
| | Intermediate B32, Intermediate A24 |
| | HPLC-1 (20-60% MeCN); Yellow solid (18.9 mg, 69%); LCMS m/z = 457 |
| | [M + H]⁺; ¹H NMR (400 MHZ, CDCl₃): 11.99 (s, 1H), 9.91 (d, 1H), 8.57 (d, |
| | 1H), 8.40 (s, 1H), 8.30 (s, 1H), 7.73 (s, 1H), 7.13 (s, 2H), 6.13-5.82 (m, 2H), |
| | 3.95 (s, 2H), 3.15 (dt, 2H), 2.84 (q, 2H), 2.17 (s, 6H), 1.18 (t, 3H). |
| 157 | 4-amino-N-(5-(1-aminocyclobutyl)pyridin-3-yl)-1-(4-(methoxymethyl)-2,6- |
| | dimethylphenyl)-6-oxo-1,6-dihydropyrimidine-5-carboxamide |
| | Intermediate B22, Intermediate A51 |
| | HPLC-7 (1-40% MeCN); White solid (23.6 mg, 57%); LCMS m/z = 449 |
| | [M + H]⁺; ¹H NMR (400 MHZ, CDCl₃): 11.92 (s, 1H), 9.89 (d, 1H), 8.65 (d, |
| | 1H), 8.34 (s, 1H), 8.05 (br s, 1H), 7.66 (s, 1H), 7.14 (s, 2H), 5.88 (d, 1H), |
| | 4.39 (s, 2H), 3.37 (s, 3H), 2.53-2.45 (m, 2H), 2.20-2.14 (m, 2H), 2.11 (s, |
| | 6H), 2.06-1.98 (m, 1H), 1.78-1.67 (m, 1H). |
| 158 | (R)-4-amino-1-(2,6-dimethyl-4-((methylsulfonyl)methyl)phenyl)-6-oxo-N- |
| | (5-(piperidin-2-yl)pyridin-3-yl)-1,6-dihydropyrimidine-5-carboxamide |
| | Intermediate B53, Intermediate A4 |
| | HPLC-1 (15-40% MeCN); White solid (12 mg, 14%); LCMS m/z = 511 |
| | [M + H]⁺; ¹H NMR (400 MHZ, CDCl₃): 11.95 (s, 1H), 9.61 (d, 1H), 8.57- |
| | 8.50 (m, 1H), 8.43 (s, 1H), 8.25 (s, 1H), 7.64 (s, 1H), 7.23 (s, 2H), 5.90 (d, |
| | 1H), 4.24-4.16 (m, 2H), 4.05-3.94 (m, 1H), 3.26 (d, 1H), 2.94-2.87 (m, 1H), |
| | 2.84 (s, 3H), 2.13 (s, 3H), 2.10 (s, 3H), 2.05-1.85 (m, 4H), 1.81-1.67 (m, |
| | 1H), 1.62-1.44 (m, 1H). |
| 159 | (R)-4-amino-1-(4-(methoxymethyl)-2,6-dimethylphenyl)-6-oxo-N-(5-(5- |
| | oxopiperazin-2-yl)pyridin-3-yl)-1,6-dihydropyrimidine-5-carboxamide or |
| | (S)-4-amino-1-(4-(methoxymethyl)-2,6-dimethylphenyl)-6-oxo-N-(5-(5- |
| | oxopiperazin-2-yl)pyridin-3-yl)-1,6-dihydropyrimidine-5-carboxamide |
| | Intermediate B22, Intermediate A74 |
| | HPLC-1 (1-25% MeCN); White solid (14.2 mg, 52%); LCMS m/z = 478 |
| | [M + H]⁺; ¹H NMR (400 MHZ, CDCl₃): 12.05 (s, 1H), 9.84 (br d, 1H), 8.59 |
| | (s, 1H), 8.29 (br d, 2H), 7.66 (s, 1H), 7.14 (s, 2H), 6.10 (br d, 2H), 4.39 (s, |
| | 2H), 4.06 (br t, 1H), 3.67 (br d, 2H), 3.37-3.39 (m, 5H), 2.10 (s, 6H). |
| 160 | (R)-4-amino-1-(4-(methoxymethyl)-2,6-dimethylphenyl)-6-oxo-N-(5-(5- |
| | oxopiperazin-2-yl)pyridin-3-yl)-1,6-dihydropyrimidine-5-carboxamide or |
| | (S)-4-amino-1-(4-(methoxymethyl)-2,6-dimethylphenyl)-6-oxo-N-(5-(5- |
| | oxopiperazin-2-yl)pyridin-3-yl)-1,6-dihydropyrimidine-5-carboxamide |
| | Intermediate B22, Intermediate A74 |
| | HPLC-1 (1-30% MeCN); White solid (16.1 mg, 59%); LCMS m/z = 478 |
| | [M + H]⁺; ¹H NMR (400 MHZ, CDCl₃): 12.04 (s, 1H), 9.84 (br d, 1H), 8.58 |
| | (d, 1H), 8.32-8.25 (m, 2H), 7.66 (s, 1H), 7.14 (s, 2H), 6.09 (br s, 2H), 4.39 |
| | (s, 2H), 4.06 (t, 1H), 3.75-3.61 (m, 2H), 3.42-3.34 (m, 5H), 2.10 (s, 6H). |
| 161 | (R)-4-amino-1-(4-fluoro-2,6-dimethylphenyl)-6-oxo-N-(5-(piperidin-2- |
| | yl)pyridin-3-yl)-1,6-dihydropyrimidine-5-carboxamide trifluoroacetate |
| | Intermediate B50, Intermediate A4 |
| | HPLC-12 (10-40% MeCN); White solid (16.1 mg, 59%); LCMS m/z = 437 |
| | [M + H]⁺; ¹H NMR (500 MHZ, DMSO-d₆): 12.23 (s, 1H), 9.50 (d, 1H), 9.01- |
| | 8.85 (m, 2H), 8.75-8.64 (m, 1H), 8.61 (d, 1H), 8.36 (s, 1H), 8.27 (s, 1H), |
| | 8.21 (s, 1H), 7.16 (d, 2H), 4.31 (t, 1H), 3.37 (d, 1H), 3.05 (q, 1H), 2.09 (s, |
| | 6H), 1.96 (d, 1H), 1.92-1.78 (m, 3H), 1.74-1.56 (m, 2H). |
| 162 | (R)-4-amino-1-(2,6-dimethyl-4-(trifluoromethyl)phenyl)-N-(5-(piperidin-2- |
| | yl)pyridin-3-yl)-6-thioxo-1,6-dihydropyrimidine-5-carboxamide |
| | trifluoroacetate |
| | Intermediate B56, Intermediate A4 |
| | HPLC-12 (10-40% MeCN); White solid (18.9 mg, 34%); LCMS m/z = 503 |
| | [M + H]⁺; ¹H NMR (500 MHZ, CDCl₃): 14.48 (s, 1H), 10.46 (s, 1H), 10.02 |
| | (s, 1H), 9.62 (s, 2H), 8.85 (s, 1H), 8.64 (s, 1H), 8.58 (s, 1H), 7.79 (s, 1H), |
| | 7.48 (s, 1H), 7.45 (s, 1H), 6.56 (s, 1H), 4.28 (d, 1H), 3.51 (d, 1H), 3.23 - |
| | 2.99 (m, 1H), 2.26 (s, 3H), 2.14 (s, 3H), 2.13-1.83 (m, 5H), 1.67 (q, 1H). |
| 163 | (R)-4-amino-1-(4-(methoxymethyl)-2,6-dimethylphenyl)-N-(5-(piperidin-2- |
| | yl)pyridin-3-yl)-6-thioxo-1,6-dihydropyrimidine-5-carboxamide |
| | trifluoroacetate |
| | Intermediate B57, Intermediate A4 |
| | HPLC-12 (10-40% MeCN); White solid (10.8 mg, 38%); LCMS m/z = 479 |
| | [M + H]⁺; ¹H NMR (500 MHZ, DMSO-d₆): 12.81 (s, 1H), 8.86 (d, 1H), 8.80 |
| | (s, 1H), 8.73 (q, 1H), 8.34 (s, 1H), 8.30 (s, 1H), 8.18 (s, 1H), 7.11 (s, 2H), |
| | 5.68 (s, 2H), 4.35 (s, 2H), 4.27 (t, 1H), 3.27 (s, 3H), 3.02 (t, 1H), 1.99 (s, |
| | 6H), 1.93-1.73 (m, 4H), 1.69-1.52 (m, 2H). |
| 164 | 4-amino-1-((S)-2-chloro-6-methylphenyl)-N-(3-methoxy-2-(2- |
| | (methylamino)ethyl)phenyl)-6-oxo-1,6-dihydropyrimidine-5-carboxamide |
| | Intermediate B3, Intermediate A64 |
| | HPLC-14 (33-53% MeCN); Yellow solid (31.4 mg, 57%); LCMS m/z = |
| | 442 [M + H]⁺; ¹H NMR (400 MHZ, MeOH-d₄): 11.69 (s, 1H), 8.08 (s, 1H), |
| | 7.57 (d, 1H), 7.54-7.48 (m, 1H), 7.46 (t, 1H), 7.43-7.38 (m, 1H), 7.29 (t, |
| | 1H), 6.87 (d, 1H), 3.88 (s, 3H), 3.16-3.10 (m, 2H), 3.08-3.00 (m, 2H), 2.61 |
| | (s, 3H), 2.26 (s, 3H). |
| 165 | 4-amino-1-(2,6-dichloro-4-ethoxyphenyl)-N-(5-(((2- |
| | (methylsulfonyl)ethyl)amino)methyl)pyridin-3-yl)-6-oxo-1,6- |
| | dihydropyrimidine-5-carboxamide |
| | Intermediate B30, Intermediate A66 |
| | HPLC-12 (10-40% MeCN); White solid (41 mg, 51%); LCMS m/z = 555 |
| | [M + H]⁺; ¹H NMR (500 MHZ, DMSO-d₆): 12.01 (s, 1H), 9.57 (d, 1H), 9.08 |
| | (s, 2H), 8.86 (d, 1H), 8.79 (d, 1H), 8.43 (s, 1H), 8.38 (d, 1H), 8.29 (t, 1H), |
| | 7.35 (s, 2H), 4.26 (s, 2H), 4.18 (q, 2H), 3.53 (dd, 2H), 3.42 (s, 2H), 3.13 (s, |
| | 3H), 1.36 (t, 3H). |
| 166 | (S)-4-amino-1-(2-methoxyphenyl)-N-(2-morpholino-3-(pyrrolidin-2- |
| | yl)phenyl)-6-oxo-1,6-dihydropyrimidine-5-carboxamide or (R)-4-amino-1- |
| | (2-methoxyphenyl)-N-(2-morpholino-3-(pyrrolidin-2-yl)phenyl)-6-oxo-1,6- |
| | dihydropyrimidine-5-carboxamide |
| | Intermediate B59, Intermediate A75 |
| | HPLC-16 (15-45% MeCN); Yellow solid (14.4 mg, 16%); LCMS m/z = |
| | 491 [M + H]⁺; ¹H NMR (400 MHZ, CDCl₃): 12.21 (s, 1H), 10.03 (d, 1H), |
| | 8.15 (dd, 1H), 7.83 (s, 1H), 7.50-7.48 (m, 1H), 7.30 (dd, 1H), 7.23-7.20 (m, |
| | 2H), 7.10-7.08 (m, 2H), 5.76 (d, 1H), 4.51 (t, 1H), 3.95-3.92 (m, 2H), 3.87 |
| | (s, 3H), 3.70 (dd, 2H), 3.27-3.25 (m, 3H), 3.01-2.96 (m, 1H), 2.93 (d, 2H), |
| | 2.26-2.20 (m, 1H), 1.90-1.88 (m, 2H), 1.68-1.65 (m, 1H). |
| 167 | (S)-4-amino-1-(2,6-dichlorophenyl)-N-(2-fluoro-3-(pyrrolidin-2-yl)phenyl)- |
| | 6-oxo-1,6-dihydropyrimidine-5-carboxamide or (R)-4-amino-1-(2,6- |
| | dichlorophenyl)-N-(2-fluoro-3-(pyrrolidin-2-yl)phenyl)-6-oxo-1,6- |
| | dihydropyrimidine-5-carboxamide |
| | Intermediate B9, Intermediate A76 |
| | HPLC-1 (5-35% MeCN); Yellow solid (7.1 mg, 31%); LCMS m/z = 462 |
| | [M + H]⁺; ¹H NMR (400 MHZ, CDCl₃): 11.83 (s, 1H), 10.08 (s, 1H), 8.30 (t, |
| | 1H), 7.73 (s, 1H), 7.55 (d, 2H), 7.46-7.42 (m, 1H), 7.16-7.09 (m, 2H), 6.03 |
| | (s, 1H), 4.52-4.50 (m, 1H), 3.22-3.14 (m, 2H), 2.27 (s, 1H), 1.96-1.80 (m, |
| | 3H). |
| 168 | (S)-4-amino-1-(2,6-dichlorophenyl)-N-(2-fluoro-3-(pyrrolidin-2-yl)phenyl)- |
| | 6-oxo-1,6-dihydropyrimidine-5-carboxamide or (R)-4-amino-1-(2,6- |
| | dichlorophenyl)-N-(2-fluoro-3-(pyrrolidin-2-yl)phenyl)-6-oxo-1,6- |
| | dihydropyrimidine-5-carboxamide |
| | Intermediate B9, Intermediate A77 |
| | HPLC-16 (30-60% MeCN); Yellow solid (3.4 mg, 16%); LCMS m/z = 462 |
| | [M + H]⁺; ¹H NMR (400 MHZ, CDCl₃): 11.70 (s, 1H), 10.00 (s, 1H), 8.18 (t, |
| | 1H), 7.64 (s, 1H), 7.45 (d, 2H), 7.37-7.33 (m, 1H), 7.09-6.99 (m, 2H), 5.90 |
| | (s, 1H), 4.38-4.34 (m, 1H), 3.12-2.96 (m, 2H), 2.19-2.11 (m, 1H), 1.86-1.79 |
| | (m, 3H). |
| 169 | (R)-4-amino-1-(2,6-dichlorophenyl)-N-(2-methoxy-3-(pyrrolidin-2- |
| | yl)phenyl)-6-oxo-1,6-dihydropyrimidine-5-carboxamide or (S)-4-amino-1- |
| | (2,6-dichlorophenyl)-N-(2-methoxy-3-(pyrrolidin-2-yl)phenyl)-6-oxo-1,6- |
| | dihydropyrimidine-5-carboxamide |
| | Intermediate B9, Intermediate A81 |
| | chiral SFC (Rilas Technologies, Regis Whelk O-1 (S,S) 250 x 21 mm, 30% |
| | EtOH + 0.25% diethylamine in CO2) |
| | HPLC-1 (1-50% MeCN); White solid (15 mg); LCMS m/z = 474 [M + H]⁺; |
| | ¹H NMR (500 MHZ, DMSO-d₆): 11.92 (s, 1H), 9.74 (d, 1H), 8.60 (d, 1H), |
| | 8.40 (s, 1H), 8.22 (dd, 1H), 7.77 (d, 2H), 7.64 (dd, 1H), 7.19 (dd, 1H), 7.05 |
| | (t, 1H), 4.27 (t, 1H), 3.63 (s, 3H), 3.07-2.97 (m, 1H), 2.87 (q, 1H), 2.11 (dq, |
| | 1H), 1.85 - 1.66 (m, 2H), 1.47-1.36 (m, 1H). |
| 170 | 4-amino-N-(3-(aminomethyl)-2-(difluoromethoxy)phenyl)-1-(2,6- |
| | dichlorophenyl)-6-oxo-1,6-dihydropyrimidine-5-carboxamide |
| | trifluoroacetate |
| | Intermediate B9, Intermediate A84 |
| | HPLC-12 (10-50% MeCN); White solid (3.4 mg, 5%); LCMS m/z = 470 |
| | [M + H]⁺; ¹H NMR (500 MHZ, DMSO-d₆): 11.95 (s, 1H), 9.62 (d, 1H), 8.71 |
| | (d, 1H), 8.42 (s, 1H), 8.37 (d, 1H), 8.16 (s, 3H), 7.77 (d, 2H), 7.64 (t, 1H), |
| | 7.41 (t, 1H), 7.28 (d, 1H), 7.01 (t, 1H), 4.05 (q, 2H). |
| 171 | 4-amino-N-(3-(aminomethyl)-2-(trifluoromethyl)phenyl)-1-(2,6- |
| | dichlorophenyl)-6-oxo-1,6-dihydropyrimidine-5-carboxamide |
| | Intermediate B9, Intermediate A78 |
| | HPLC-1 (10-40% MeCN); White solid (6.6 mg, 36%); LCMS m/z = 472 |
| | [M + H]⁺; ¹H NMR (400 MHZ, DMSO-d₆): 11.61 (br d, 1H), 9.62 (br s, 1H), |
| | 8.67 (br s, 1H), 8.53-8.31 (m, 1H), 7.75 (d, 2H), 7.74-7.67 (m, 1H), 7.65- |
| | 7.60 (m, 1H), 7.55 (d, 1H), 3.90 (d, 2H). |
| 172 | (R)-4-amino-1-(2,6-dichloro-4-methoxyphenyl)-6-oxo-N-(6-(piperidin-2- |
| | yl)pyridazin-4-yl)-1,6-dihydropyrimidine-5-carboxamide or (S)-4-amino-1- |
| | (2,6-dichloro-4-methoxyphenyl)-6-oxo-N-(6-(piperidin-2-yl)pyridazin-4- |
| | yl)-1,6-dihydropyrimidine-5-carboxamide |
| | Intermediate B10, Intermediate A79 |
| | HPLC-2 (5-35% MeCN); Brown solid (13.7 mg, 51%); LCMS m/z = 490 |
| | [M + H]⁺; ¹H NMR (400 MHZ, CDCl₃): 12.07 (s, 1H), 9.89 (d, 1H), 9.17 (d, |
| | 1H), 8.10 (d, 1H), 7.75 (s, 1H), 7.07 (s, 2H), 6.28 (d, 1H), 4.14-4.07 (m, |
| | 1H), 3.89 (s, 3H), 3.31 (d, 1H), 2.96-2.87 (m, 1H), 2.03 (d, 1H), 1.94 (s, |
| | 1H), 1.72 (s, 1H), 1.66 (d, 1H), 1.63-1.55 (m, 2H). |
| 173 | (S)-4-amino-1-(2-chloro-6-hydroxyphenyl)-6-oxo-N-(5-(piperidin-2- |
| | yl)pyridin-3-yl)-1,6-dihydropyrimidine-5-carboxamide |
| | Intermediate B60, Intermediate A5 |
| | HPLC-1 (1-40% MeCN); Yellow oil (8.7 mg, 45%); LCMS m/z = 441 |
| | [M + H]⁺; ¹H NMR (400 MHZ, DMSO-d₆): 12.09-12.04 (m, 1H), 9.53 (br d, |
| | 1H), 8.72-8.68 (m, 1H), 8.54 (br d, 1H), 8.28-8.26 (m, 2H), 8.12 (s, 1H), |
| | 7.37-7.31 (m, 1H), 7.08-7.01 (m, 2H), 3.80 (br d, 2H), 3.13 (br d, 1H), 2.80- |
| | 2.72 (m, 1H), 1.84-1.74 (m, 2H), 1.62 (br s, 1H), 1.49 (br d, 3H). |

### Example 174 and 175. Synthesis of 4-amino-N-(5-((R)-1-aminoethyl)pyridin-3-yl)-1-(4-((R)-1-methoxyethyl)-2,6-dimethylphenyl)-6-oxo-1,6-dihydropyrimidine-5-carboxamide or 4-amino-N-(5-((S)-1-aminoethyl)pyridin-3-yl)-1-(4-((R)-1-methoxyethyl)-2,6-dimethylphenyl)-6-oxo-1,6-dihydropyrimidine-5-carboxamide or 4-amino-N-(5-((R)-1-aminoethyl)pyridin-3-yl)-1-(4-((S)-1-methoxyethyl)-2,6-dimethylphenyl)-6-oxo-1,6-dihydropyrimidine-5-carboxamide or 4-amino-N-(5-((S)-1-aminoethyl)pyridin-3-yl)-1-(4-((S)-1-methoxyethyl)-2,6-dimethylphenyl)-6-oxo-1,6-dihydropyrimidine-5-carboxamide

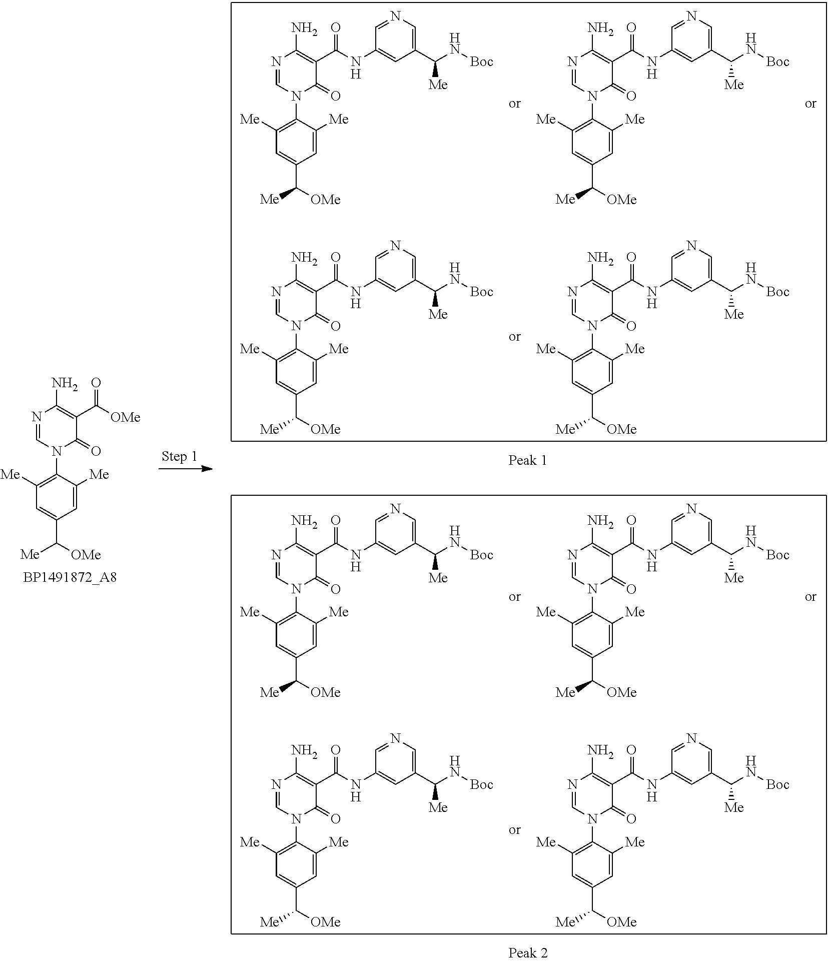

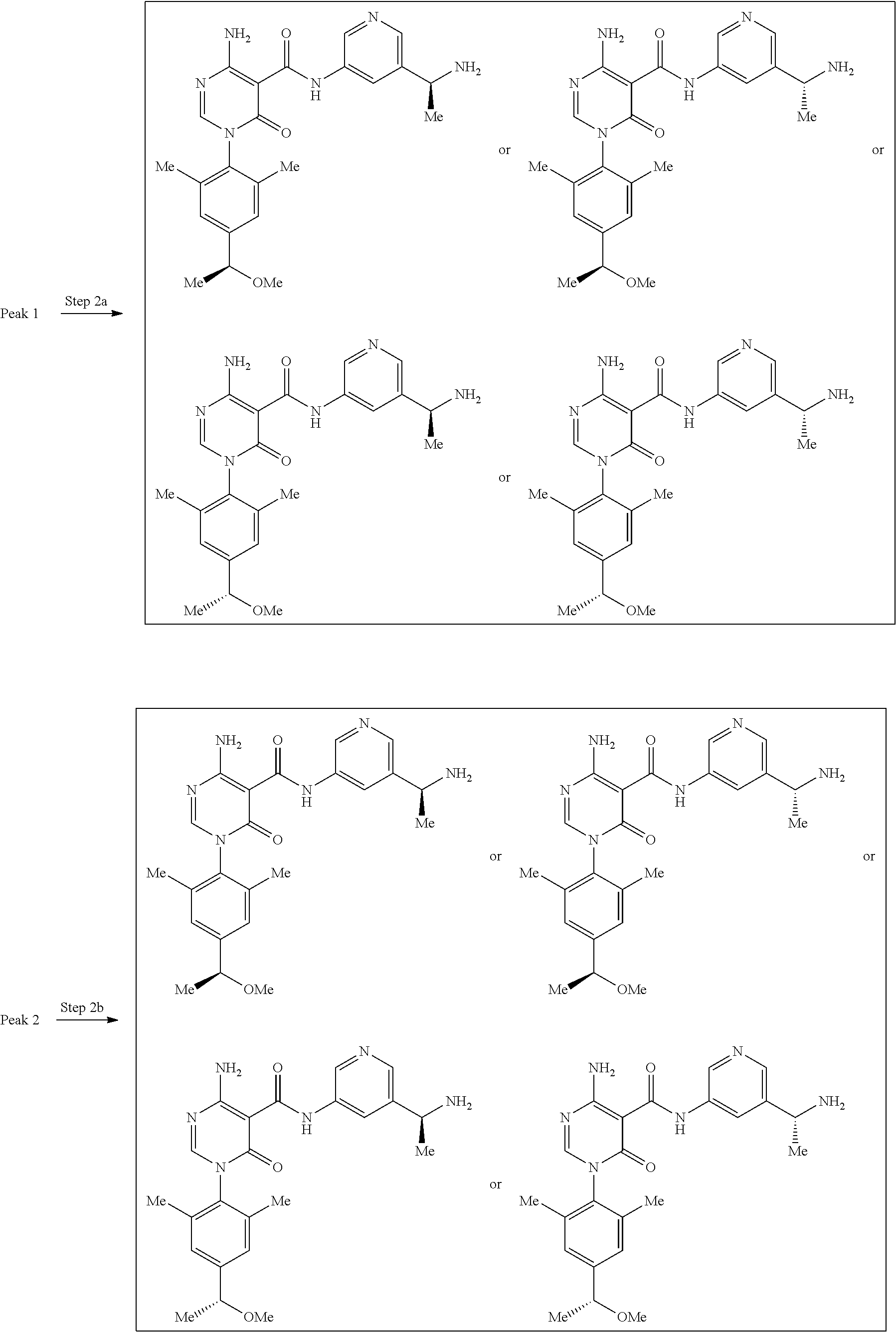

Step 1. To a mixture of methyl 4-amino-1-(4-(1-methoxyethyl)-2,6-dimethylphenyl)-6-oxo-1,6-dihydropyrimidine-5-carboxylate (Intermediate B51, 50 mg, 0.151 mmol) and tert-butyl(S) (1-(5-aminopyridin-3-yl)ethyl)carbamate or tert-butyl (R)-(1-(5-aminopyridin-3-yl)ethyl)carbamate (Intermediate A16, 53.7 mg, 0.226 mmol) in toluene (3 mL) was added AlMe₃ (2 M in toluene, 0.226 mL) at 0° C. and the mixture stirred at 40° C. for 1 h under N₂. The reaction mixture was diluted with 1M NaOH (20 mL) and extracted with EtOAc (3×20 mL). The combined organics were washed with brine (2×10 mL), dried (Na₂SO₄) and concentrated under reduced pressure to give a residue. The residue was purified by prep-TLC (50% EtOAc/PE) followed by prep-SFC (DAICEL CHIRALPAK IE, 250×30 mm, 10 μm); 40% [Heptane-(4:1 IPA: MeCN)] in CO₂) to afford Peak 1 as a white solid (10 mg, 12%) and Peak 2 as a white solid (10 mg, 12%) which were independently taken through to the next step.

### Step 2a. Synthesis of 4-amino-N-(5-((R)-1-aminoethyl)pyridin-3-yl)-1-(4-((R)-1-methoxyethyl)-2,6-dimethylphenyl)-6-oxo-1,6-dihydropyrimidine-5-carboxamide or 4-amino-N-(5-((S)-1-aminoethyl)pyridin-3-yl)-1-(4-((R)-1-methoxyethyl)-2,6-dimethylphenyl)-6-oxo-1,6-dihydropyrimidine-5-carboxamide or 4-amino-N-(5-((R)-1-aminoethyl)pyridin-3-yl)-1-(4-((S)-1-methoxyethyl)-2,6-dimethylphenyl)-6-oxo-1,6-dihydropyrimidine-5-carboxamide or 4-amino-N-(5-((S)-1-aminoethyl)pyridin-3-yl)-1-(4-((S)-1-methoxyethyl)-2,6-dimethylphenyl)-6-oxo-1,6-dihydropyrimidine-5-carboxamide

The compound of Peak 1 (Part 1 Peak 1, 10 mg, 18.64 μmol) in DCM (2 mL) and TFA (2 mL) was stirred at 25° C. for 0.5 h and the reaction mixture concentrated under N₂ and under reduced pressure. The residue was purified by prep-HPLC-1 (1-50% MeCN) to give Example 174. White solid (4.2 mg, 46%). LCMS m/z=437 [M+H]; ¹H NMR (400 MHz, CDCl₃): 1.46 (d, 3H), 1.53 (br d, 3H), 2.19 (d, 6H), 3.31 (s, 3H), 4.31 (q, 2H), 6.26 (br d, 1H), 7.18 (br d, 2H), 7.75 (s, 1H), 8.33 (br d, 3H), 8.58 (s, 1H), 9.85 (br s, 1H), 12.03 (s, 1H).

### Step 2b. Synthesis of 4-amino-N-(5-((R)-1-aminoethyl)pyridin-3-yl)-1-(4-((R)-1-methoxyethyl)-2,6-dimethylphenyl)-6-oxo-1,6-dihydropyrimidine-5-carboxamide or 4-amino-N-(5-((S)-1-aminoethyl)pyridin-3-yl)-1-(4-((R)-1-methoxyethyl)-2,6-dimethylphenyl)-6-oxo-1,6-dihydropyrimidine-5-carboxamide or 4-amino-N-(5-((R)-1-aminoethyl)pyridin-3-yl)-1-(4-((S)-1-methoxyethyl)-2,6-dimethylphenyl)-6-oxo-1,6-dihydropyrimidine-5-carboxamide or 4-amino-N-(5-((S)-1-aminoethyl)pyridin-3-yl)-1-(4-((S)-1-methoxyethyl)-2,6-dimethylphenyl)-6-oxo-1,6-dihydropyrimidine-5-carboxamide

The compound of Peak 2 (Part 1, Peak 2) was deprotected in an analogous manner as described for Peak 1 to give Example 175. White solid (4.2 mg, 46%). LCMS m/z=437 [M+H]⁺;]⁺; ¹H NMR (400 MHz, CDCl₃): 1.37 (d, 3H) 1.46 (br d, 3H) 2.10 (d, 6H) 3.21 (s, 3H) 4.13-4.33 (m, 2H) 6.19 (br s, 1H) 7.08 (s, 2H) 7.65 (s, 1H) 8.26 (br d, 2H) 8.47 (s, 1H) 9.74 (br s, 1H) 11.93 (s, 1H).

### Example 176. Synthesis of (R)-4-amino-N-(5-(1-aminobutyl)pyridin-3-yl)-1-(4-(methoxymethyl)-2,6-dimethylphenyl)-6-oxo-1,6-dihydropyrimidine-5-carboxamide

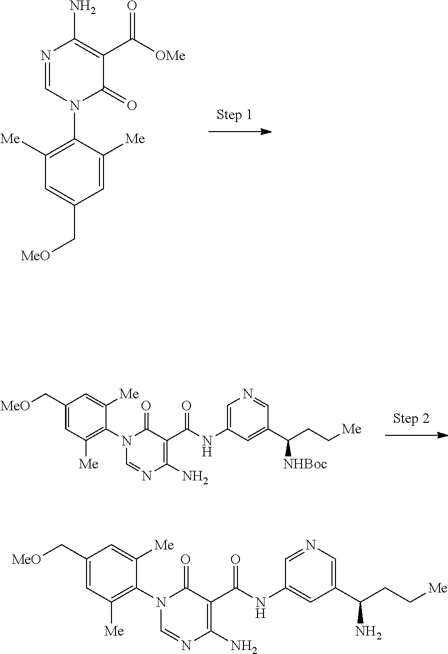 [SMILES: COCc1cc(C)c(-n2cnc(N)c(C(=O)OC)c2=O)c(C)c1 ; CCC[C@@H](NC(=O)OC(C)(C)C)c1cncc(NC(=O)c2c(N)ncn(-c3c(C)cc(COC)cc3C)c2=O)c1 ; CCC[C@@H](N)c1cncc(NC(=O)c2c(N)ncn(-c3c(C)cc(COC)cc3C)c2=O)c1]

### Step 1. Synthesis of tert-butyl (R)-(1-(5-(4-amino-1-(4-(methoxymethyl)-2,6-dimethylphenyl)-6-oxo-1,6-dihydropyrimidine-5-carboxamido)pyridin-3-yl)butyl)carbamate

AlMe₃ (2 M, 142 L) was added dropwise to a solution of methyl 4-amino-1-(4-(methoxymethyl)-2,6-dimethylphenyl)-6-oxo-1,6-dihydropyrimidine-5-carboxylate (Intermediate B22, 30 mg, 0.095 mmol) and tert-butyl (R)-(1-(5-aminopyridin-3-yl)butyl)carbamate (Intermediate A7, 37.6 mg, 0.142 mmol) in toluene (2 mL) at 0° C. and the mixture stirred at 40° C. for 1 h under N₂. The reaction mixture was quenched with IN NaOH aq. (2 mL) at 0° C. and extracted with EtOAc (4×5 mL). The combined organics were washed with brine (5 mL) and dried (Na₂SO₄) and evaporated to dryness to afford the title compound as a yellow oil (50 mg, crude). LCMS m/z=551 [M+H]⁺;

### Step 2. Synthesis of (R)-4-amino-N-(5-(1-aminobutyl)pyridin-3-yl)-1-(4-(methoxymethyl)-2,6-dimethylphenyl)-6-oxo-1,6-dihydropyrimidine-5-carboxamide

Tert-butyl (R)-(1-(5-(4-amino-1-(4-(methoxymethyl)-2,6-dimethylphenyl)-6-oxo-1,6-dihydropyrimidine-5-carboxamido)pyridin-3-yl)butyl)carbamate (Part 1, 50 mg, 0.091 mmol) was dissolved into DCM (1 mL) and TFA (1 mL) and the mixture stirred at 25° C. for 0.5 h. The reaction mixture was concentrated under reduced pressure and the residue purified by prep-HPLC-8 (20-55% MeCN) to give the title compound as a pale yellow solid (12.3 mg, 27%). LCMS m/z=451 [M+H]⁺; ¹H NMR (400 MHz, CDCl₃): 0.91 (t, 3H) 1.19-1.39 (m, 2H) 1.67 (d, 2H) 2.18 (s, 6H) 3.45 (s, 3H) 3.94 (t, 1H) 4.46 (s, 2H) 6.07 (br d, 1H) 7.21 (s, 2H) 7.73 (s, 1H) 8.12 (s, 1H) 8.26 (s, 1H) 8.67 (d, 1H) 9.97 (d, 1H) 12.01 (s, 1H).

### Example 177. Synthesis of (S)-4-amino-N-(5-(1-amino-2-ethoxyethyl)pyridin-3-yl)-1-(4-(methoxymethyl)-2,6-dimethylphenyl)-6-oxo-1,6-dihydropyrimidine-5-carboxamide or (R)-4-amino-N-(5-(1-amino-2-ethoxyethyl)pyridin-3-yl)-1-(4-(methoxymethyl)-2,6-dimethylphenyl)-6-oxo-1,6-dihydropyrimidine-5-carboxamide

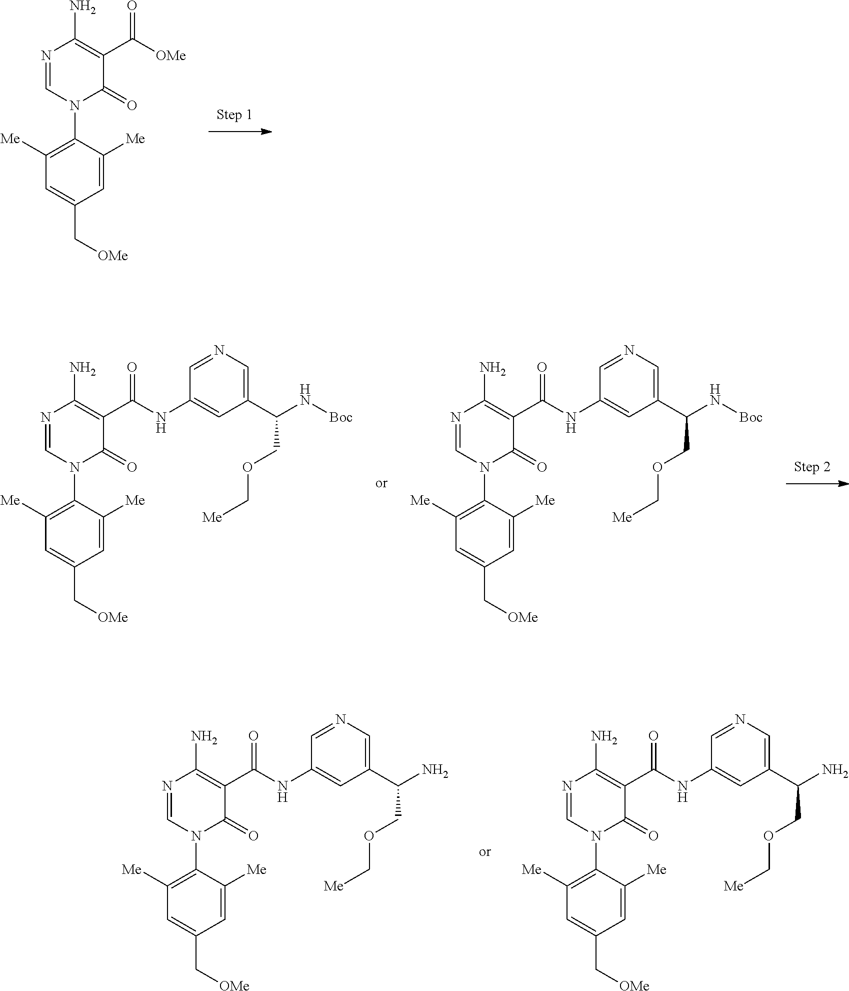 [SMILES: COCc1cc(C)c(-n2cnc(N)c(C(=O)OC)c2=O)c(C)c1 ; CCOC[C@@H](NC(=O)OC(C)(C)C)c1cncc(NC(=O)c2c(N)ncn(-c3c(C)cc(COC)cc3C)c2=O)c1 ; CCOC[C@H](NC(=O)OC(C)(C)C)c1cncc(NC(=O)c2c(N)ncn(-c3c(C)cc(COC)cc3C)c2=O)c1 ; CCOC[C@@H](N)c1cncc(NC(=O)c2c(N)ncn(-c3c(C)cc(COC)cc3C)c2=O)c1 ; CCOC[C@H](N)c1cncc(NC(=O)c2c(N)ncn(-c3c(C)cc(COC)cc3C)c2=O)c1]

### Step 1. Synthesis of tert-butyl(S)-(1-(5-(4-amino-1-(4-(methoxymethyl)-2,6-dimethylphenyl)-6-oxo-1,6-dihydropyrimidine-5-carboxamido)pyridin-3-yl)-2-ethoxyethyl)carbamate or tert-butyl (R)-(1-(5-(4-amino-1-(4-(methoxymethyl)-2,6-dimethylphenyl)-6-oxo-1,6-dihydropyrimidine-5-carboxamido)pyridin-3-yl)-2-ethoxyethyl)carbamate

To a solution of methyl 4-amino-1-(4-(methoxymethyl)-2,6-dimethylphenyl)-6-oxo-1,6-dihydropyrimidine-5-carboxylate (Intermediate B22, 50 mg, 0.158 mmol) and tert-butyl(S)-(1-(5-aminopyridin-3-yl)-2-ethoxyethyl)carbamate or tert-butyl (R)-(1-(5-aminopyridin-3-yl)-2-ethoxyethyl)carbamate (Intermediate A11, 66.5 mg, 0.236 mmol) in toluene (3 mL) was added AlMe₃ (2 M in toluene, 394 μL) at 0° C. and the mixture stirred at 60° C. for 1 h under N₂. The reaction mixture was quenched by addition H₂O (0.5 mL) and TFA (0.5 mL) at 0° C., filtered and concentrated under reduced pressure to give the title compound as a white solid (75 mg, 0.132 mmol, 84%). LCMS m/z=451 [M+H].

### Step 2. Synthesis of (S)-4-amino-N-(5-(1-amino-2-ethoxyethyl)pyridin-3-yl)-1-(4-(methoxymethyl)-2,6-dimethylphenyl)-6-oxo-1,6-dihydropyrimidine-5-carboxamide or (R)-4-amino-N-(5-(1-amino-2-ethoxyethyl)pyridin-3-yl)-1-(4-(methoxymethyl)-2,6-dimethylphenyl)-6-oxo-1,6-dihydropyrimidine-5-carboxamide

A mixture of tert-butyl(S)-(1-(5-(4-amino-1-(4-(methoxymethyl)-2,6-dimethylphenyl)-6-oxo-1,6-dihydropyrimidine-5-carboxamido)pyridin-3-yl)-2-ethoxyethyl)carbamate or tert-butyl (R)-(1-(5-(4-amino-1-(4-(methoxymethyl)-2,6-dimethylphenyl)-6-oxo-1,6-dihydropyrimidine-5-carboxamido)pyridin-3-yl)-2-ethoxyethyl)carbamate (Step 1, 70 mg, 0.124 mmol) in DCM (1 mL) and TFA (2 mL) was stirred at 25° C. for 1 h. The reaction mixture was concentrated under N₂ and the residue purified by prep-HPLC-9 (10-45% MeCN) to afford the title compound as a brown solid (9.90 mg, 16%). LCMS m/z=467 [M+H]⁺; ¹H NMR (400 MHz, CDCl₃): 11.95 (s, 1H) 9.84 (d, 1H) 8.62 (d, 1H) 8.25 (s, 1H) 8.17 (s, 1H) 7.66 (s, 1H) 7.13 (s, 2H) 5.94 (d, 1H) 4.39 (s, 2H) 4.18 (dd, 1H) 3.40-3.55 (m, 4H) 3.37 (s, 3H) 2.10 (s, 6H) 1.13 (t, 3H).

### Example 178. Synthesis of 4-amino-1-(2,6-dichloro-4-ethoxyphenyl)-N-(5-((ethylamino)methyl)pyridin-3-yl)-6-oxo-1,6-dihydropyrimidine-5-carboxamide

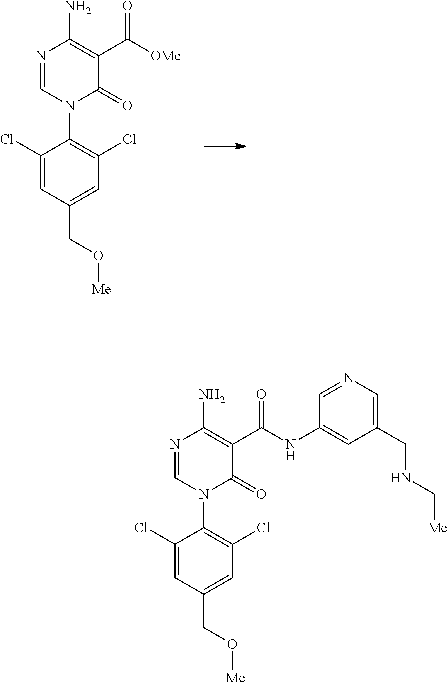 [SMILES: COCc1cc(Cl)c(-n2cnc(N)c(C(=O)OC)c2=O)c(Cl)c1 ; CCNCc1cncc(NC(=O)c2c(N)ncn(-c3c(Cl)cc(COC)cc3Cl)c2=O)c1]

AlMe₃ (2M in toluene, 209 μL) was added to a mixture of methyl 4-amino-1-(2,6-dichloro-4-ethoxyphenyl)-6-oxo-1,6-dihydropyrimidine-5-carboxylate (Intermediate B30, 50 mg, 0.140 mmol) and 5-((ethylamino)methyl)pyridin-3-amine (Intermediate A12, 31.7 mg, 0.209 mmol) in toluene (2 mL) and the mixture was stirred at 0° C. for 5 min and then stirred at 100° C. for 0.5 hr under N₂. The reaction mixture was quenched by addition IN NaOH aq. (2 mL) at 0° C. and extracted with EtOAc (4×5 mL). The combined organics were washed with sat. aq. NaCl (5 mL), dried (Na₂SO₄) and concentrated under reduced pressure. The residue was purified by prep-HPLC-8 (5-40% MeCN) to give the title compound as a white solid (45.2 mg, 61%). LCMS m/z=477 [M+H]⁺; ¹H NMR (400 MHz, CDCl₃): 11.84 (s, 1H), 9.96 (d, 1H), 8.58 (d, 1H), 8.47-8.41 (m, 2H), 8.32 (s, 1H), 7.72 (s, 1H), 7.04 (s, 2H), 6.20 (d, 1H), 4.08 (q, 2H), 3.97 (s, 2H), 2.83 (q, 2H), 1.46 (t, 3H), 1.26-1.15 (m, 3H).

### Example 179. Synthesis of 4-amino-N-(5-(1-aminocyclobutyl)pyridin-3-yl)-1-(2,6-dichloro-4-methoxyphenyl)-6-oxo-1,6-dihydropyrimidine-5-carboxamide

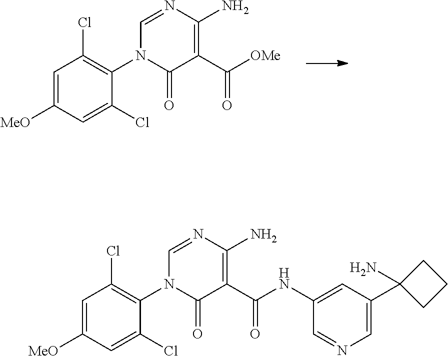 [SMILES: COC(=O)c1c(N)ncn(-c2c(Cl)cc(OC)cc2Cl)c1=O ; COc1cc(Cl)c(-n2cnc(N)c(C(=O)Nc3cncc(C4(N)CCC4)c3)c2=O)c(Cl)c1]

### Step 1. Synthesis of tert-butyl(1-(5-(4-amino-1-(2,6-dichloro-4-methoxyphenyl)-6-oxo-1,6-dihydropyrimidine-5-carboxamido)pyridin-3-yl)cyclobutyl)carbamate

AlMe₃ (2 M, 262 μL) was added to a solution of methyl 4-amino-1-(2,6-dichloro-4-methoxyphenyl)-6-oxo-1,6-dihydropyrimidine-5-carboxylate (Intermediate B10, 60.0 mg, 0.174 mmol) and tert-butyl(1-(5-aminopyridin-3-yl)cyclobutyl)carbamate (Intermediate A51, 51.0 mg, 0.192 mmol) in toluene (15 mL) at 0° C. under N₂ and the mixture heated at 80° C. for 3 h. The reaction mixture was diluted with water (20 mL) and extracted with EtOAc (3×30 mL). The combined organics were dried (Na₂SO₄) and concentrated under reduced pressure. The residue was purified by prep-TLC (SiO₂, 50% EtOAc/PE) to afford the title compound as a yellow solid (30 mg, 30%). LCMS m/z=575 [M+H]⁺.

### Step 2. Synthesis of 4-amino-N-(5-(1-aminocyclobutyl)pyridin-3-yl)-1-(2,6-dichloro-4-methoxyphenyl)-6-oxo-1,6-dihydropyrimidine-5-carboxamide

A mixture of tert-butyl(1-(5-(4-amino-1-(2,6-dichloro-4-methoxyphenyl)-6-oxo-1,6-dihydropyrimidine-5-carboxamido)pyridin-3-yl)cyclobutyl)carbamate (30.0 mg, 52.1 μmol) in hydrochloric/dioxane (4 M, 6 mL) was stirred at 25° C. for 10 min. The reaction mixture was concentrated under reduced pressure and the residue purified by prep-HPLC-1 (12-42% MeCN) to afford the title compound as a white solid (8.20 mg, 30% yield). LCMS m/z=475 [M+H]⁺; ¹H NMR (400 MHz, MeOH-d₄): 8.81-8.77 (m, 1H), 8.53 (s, 1H), 8.40-8.35 (m, 1H), 8.22 (t, 1H), 8.08 (s, 1H), 7.23 (s, 2H), 3.90 (s, 3H), 2.73-2.62 (m, 2H), 2.46-2.38 (m, 2H), 2.23-2.12 (m, 1H), 1.96-1.82 (m, 1H).

### Example 180. Synthesis of 4-amino-1-(2,6-dichlorophenyl)-N-(3-((2R,5R)-5-methylpyrrolidin-2-yl)phenyl)-6-oxo-1,6-dihydropyrimidine-5-carboxamide or 4-amino-1-(2,6-dichlorophenyl)-N-(3-((2S,5S)-5-methylpyrrolidin-2-yl)phenyl)-6-oxo-1,6-dihydropyrimidine-5-carboxamide

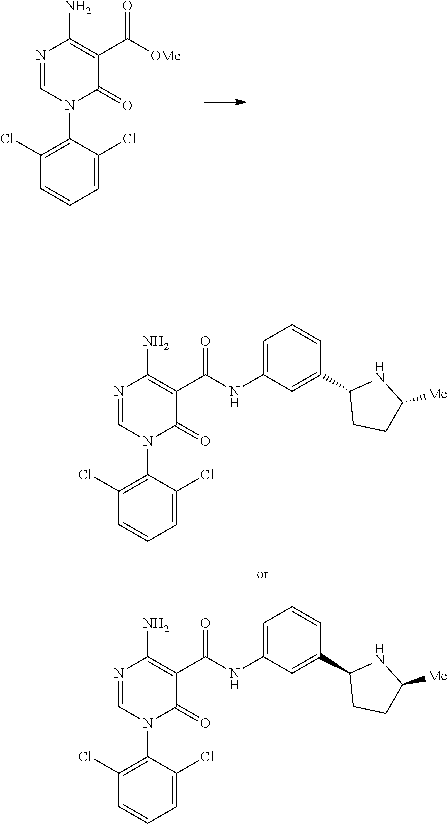 [SMILES: COC(=O)c1c(N)ncn(-c2c(Cl)cccc2Cl)c1=O ; C[C@@H]1CC[C@H](c2cccc(NC(=O)c3c(N)ncn(-c4c(Cl)cccc4Cl)c3=O)c2)N1 ; C[C@H]1CC[C@@H](c2cccc(NC(=O)c3c(N)ncn(-c4c(Cl)cccc4Cl)c3=O)c2)N1]

A dry flask containing methyl 4-amino-1-(2,6-dichlorophenyl)-6-oxo-1,6-dihydropyrimidine-5-carboxylate (Intermediate B9, 50.3 mg, 0.160 mmol) and rel-tert-butyl(2S,5S)-2-(3-aminophenyl)-5-methylpyrrolidine-1-carboxylate (Intermediate A72, 64.1 mg, 0.192 mmol) was evacuated and backfilled three times with nitrogen. Anhydrous toluene (3 mL) was added and the solution cooled to 0° C. and trimethylaluminum solution (2.0 M in toluene, 0.240 mL, 0.480 mmol) was added. The flask was removed from the ice bath and the reaction stirred at rt for 10 min and then heated to 100° C. for 1 h. The reaction was cooled to rt, quenched with methanol (2 mL) and TFA (0.2 mL), poured into water (30 mL) and extracted with DCM (4×10 mL). The combined organics were washed with saturated aqueous sodium bicarbonate, water, and brine, dried (MgSO₄) and evaporated to dryness in vacuo. The residue was dissolved in DCM (2 mL) and TFA (1 mL) and the solution stirred at rt for 3 h. The reaction mixture was evaporated to dryness and the residue purified by prep-HPLC-10 (10-60%) followed by chiral SFC (Regis Whelk O-1 (S,S) 4.6×100 mm, 3 μm, 45% EtOH+0.25% diethylamine in CO₂) to afford:

Peak 1, Example 180. 4-amino-1-(2,6-dichlorophenyl)-N-(3-((2R,5R)-5-methylpyrrolidin-2-yl)phenyl)-6-oxo-1,6-dihydropyrimidine-5-carboxamide or 4-amino-1-(2,6-dichlorophenyl)-N-(3-((2S,5S)-5-methylpyrrolidin-2-yl)phenyl)-6-oxo-1,6-dihydropyrimidine-5-carboxamide (white powder, 17.9 mg). LCMS m/z=458 [M+H]⁺; ¹H NMR (500 MHz, DMSO-d₆): 11.90 (s, 1H), 9.68 (d, 1H), 9.43 (s, 1H), 8.73 (d, 1H), 8.46 (s, 1H), 8.43-8.28 (m, 1H), 7.84 (dd, 1H), 7.77 (d, 2H), 7.71-7.58 (m, 2H), 7.40 (t, 1H), 7.15 (d, 1H), 4.68-4.55 (m, 1H), 3.81-3.65 (m, 1H), 2.40 (ddt, 1H), 2.25 (dq, 1H), 2.13 (dtd, 1H), 1.72 (dq, 1H), 1.34 (d, 3H).

### Example 181-186

The title compounds were prepared from the appropriate ester and the appropriate amine using an analogous 2-Step method as described for Example 179.

| Example | |
|---|---|
| No | Name, Structure, Starting Material (SM), Data |
| 181 | 4-amino-N-(5-(1-aminocyclopropyl)pyridin-3-yl)-1-(2,6-dichloro-4- |
| | methoxyphenyl)-6-oxo-1,6-dihydropyrimidine-5-carboxamide |
| | 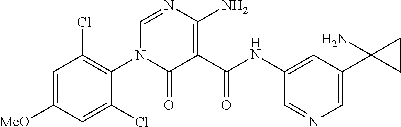 [SMILES: COc1cc(Cl)c(-n2cnc(N)c(C(=O)Nc3cncc(C4(N)CC4)c3)c2=O)c(Cl)c1] |
| | Intermediate B10, Intermediate A40 |
| | HPLC-1 (10-40% MeCN); White solid (9.7 mg, 24%); LCMS m/z = 461 |
| | [M + H]⁺; ¹H NMR (400 MHz, MeOH-d₄): 8.70 (s, 1H), 8.27 (s, 1H), 8.07 (s, |
| | 1H), 8.06-8.03 (m, 1H), 7.22 (s, 2H), 3.90 (s, 3H), 1.18-1.13 (m, 2H), 1.11-1.05 |
| | (m, 2H). |
| 182 | 4-amino-N-(5-(4-aminotetrahydro-2H-pyran-4-yl)pyridin-3-yl)-1-(2,6-dichloro- |
| | 4-methoxyphenyl)-6-oxo-1,6-dihydropyrimidine-5-carboxamide |
| | 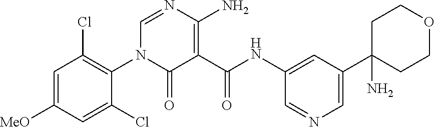 [SMILES: COc1cc(Cl)c(-n2cnc(N)c(C(=O)Nc3cncc(C4(N)CCOCC4)c3)c2=O)c(Cl)c1] |
| | Intermediate B10, Intermediate A63 |
| | HPLC-2 (9-39% MeCN); White solid (10.1 mg, 48%); LCMS m/z = 505 |
| | [M + H]⁺; ¹H NMR (400 MHz, DMSO-d₆): 11.82 (s, 1H), 9.79-9.49 (m, 1H), |
| | 8.77 (s, 1H), 8.74-8.66 (m, 1H), 8.45 (s, 1H), 8.40 (d, 1H), 8.04 (d, 1H), 7.37 |
| | (d, 2H), 3.89 (s, 3H), 3.87-3.81 (m, 2H), 3.62 (d, 2H), 2.00-1.92 (m, 2H), 1.53 |
| | (d, 2H). |
| 183 | 4-amino-N-(5-(1-aminocyclobutyl)pyridin-3-yl)-1-(2,6-dichloro-4- |
| | ethoxyphenyl)-6-oxo-1,6-dihydropyrimidine-5-carboxamide |
| | 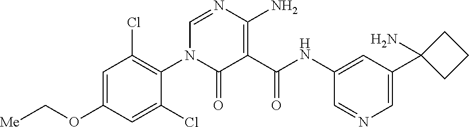 [SMILES: CCOc1cc(Cl)c(-n2cnc(N)c(C(=O)Nc3cncc(C4(N)CCC4)c3)c2=O)c(Cl)c1] |
| | Intermediate B30, Intermediate A51 |
| | HPLC-5 (30-50% MeCN); White solid (20 mg, 80%); LCMS m/z = 489 |
| | [M + H]⁺; ¹H NMR (400 MHz, MeOH-d₄): 12.16 (s, 1H), 8.93 (s, 1H), 8.42 (s, |
| | 1H), 8.36 (s, 1H), 8.11 (s, 1H), 7.22 (s, 2H), 4.18-4.11 (m, 2H), 2.91-2.79 (m, |
| | 2H), 2.70-2.60 (m, 2H), 2.34-2.20 (m, 1H), 2.13-1.90 (m, 1H), 1.47-1.40 (t, |
| | 3H). |
| 184 | 4-amino-1-(4-((R)-1-fluoroethyl)-2,6-dimethylphenyl)-6-oxo-N-(5-((R)- |
| | piperidin-2-yl)pyridin-3-yl)-1,6-dihydropyrimidine-5-carboxamide or 4-amino- |
| | 1-(4-((S)-1-fluoroethyl)-2,6-dimethylphenyl)-6-oxo-N-(5-((R)-piperidin-2- |
| | yl)pyridin-3-yl)-1,6-dihydropyrimidine-5-carboxamide |
| | 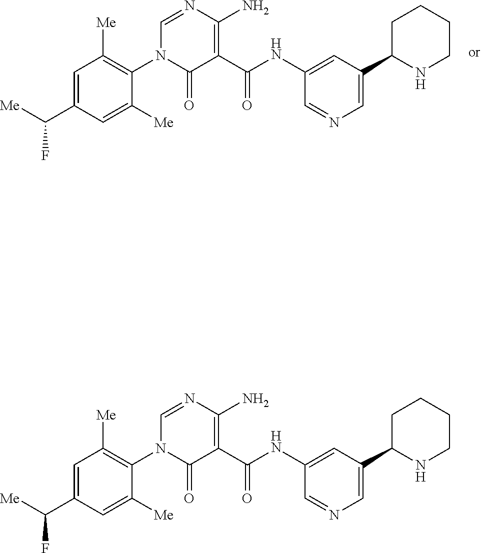 [SMILES: Cc1cc([C@@H](C)F)cc(C)c1-n1cnc(N)c(C(=O)Nc2cncc([C@H]3CCCCN3)c2)c1=O ; Cc1cc([C@H](C)F)cc(C)c1-n1cnc(N)c(C(=O)Nc2cncc([C@H]3CCCCN3)c2)c1=O] |
| | Intermediate B54, Intermediate A4 |
| | HPLC-13 (35-60% MeCN); Yellow solid (20.4 mg, 58%); LCMS m/z = 465 |
| | [M + H]⁺; ¹H NMR (400 MHz, CDCl₃): 11.96 (s, 1H), 10.11-9.89 (m, 1H), 8.70 |
| | (d, 1H), 8.34 (d, 1H), 8.19 (s, 1H), 7.74 (s, 1H), 7.25 (s, 1H), 7.21 (s, 1H), 5.97 |
| | (d, 1H), 5.77-5.51 (m, 1H), 3.73-3.65 (m, 1H), 3.21 (d, 1H), 2.27-2.17 (m, 6H), |
| | 1.92 (d, 2H), 1.87-1.78 (m, 2H), 1.71 (d, 2H), 1.65 (d, 1H), 1.63-1.47 (m, 3H). |
| 185 | 4-amino-1-(4-((R)-1-fluoroethyl)-2,6-dimethylphenyl)-6-oxo-N-(5-((R)- |
| | piperidin-2-yl)pyridin-3-yl)-1,6-dihydropyrimidine-5-carboxamide or 4-amino- |
| | 1-(4-((S)-1-fluoroethyl)-2,6-dimethylphenyl)-6-oxo-N-(5-((R)-piperidin-2- |
| | yl)pyridin-3-yl)-1,6-dihydropyrimidine-5-carboxamide |
| | 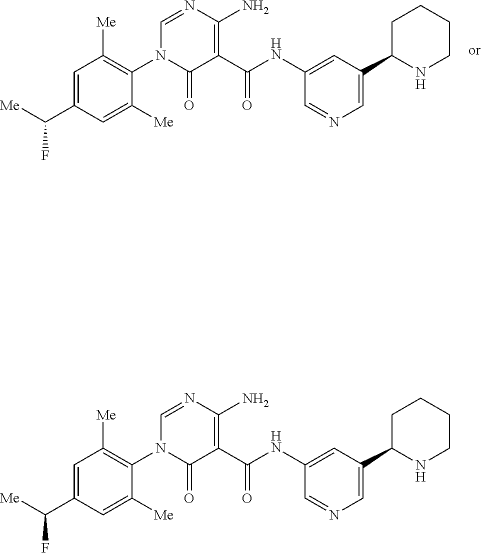 [SMILES: Cc1cc([C@@H](C)F)cc(C)c1-n1cnc(N)c(C(=O)Nc2cncc([C@H]3CCCCN3)c2)c1=O ; Cc1cc([C@H](C)F)cc(C)c1-n1cnc(N)c(C(=O)Nc2cncc([C@H]3CCCCN3)c2)c1=O] |
| | Intermediate B54, Intermediate A4 |
| | HPLC-13 (30-60% MeCN); Grey solid (9.6 mg, 27%); LCMS m/z = 465 |
| | [M + H]⁺; ¹H NMR (400 MHz, CDCl₃): 11.87 (s, 1H), 9.92 (d, 1H), 8.62 (d, 1H), |
| | 8.24 (d, 1H), 8.07 (s, 1H), 7.65 (s, 1H), 7.16 (s, 1H), 7.12 (s, 1H), 5.93 (d, 1H), |
| | 5.65-5.46 (m, 1H), 3.59-3.54 (m, 1H), 3.12 (d, 1H), 2.75-2.67 (m, 1H), 2.12 (d, |
| | 6H), 1.71 (s, 2H), 1.62 (d, 2H), 1.56 (d, 2H), 1.49-1.40 (m, 3H). |
| 186 | (R)-4-amino-1-(4-(1,1-difluoroethyl)-2,6-dimethylphenyl)-6-oxo-N-(5- |
| | (piperidin-2-yl)pyridin-3-yl)-1,6-dihydropyrimidine-5-carboxamide |
| | 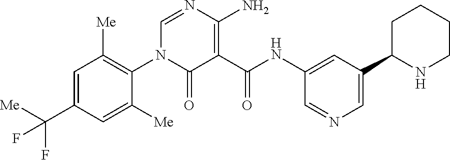 [SMILES: Cc1cc(C(C)(F)F)cc(C)c1-n1cnc(N)c(C(=O)Nc2cncc([C@H]3CCCCN3)c2)c1=O] |
| | Intermediate B55, Intermediate A4 |
| | HPLC-13 (25-65% MeCN); Grey solid (47.7 mg, 93%); LCMS m/z = 483 |
| | [M + H]⁺; ¹H NMR (400 MHz, CDCl₃): 11.93 (s, 1H), 10.04 (d, 1H), 8.70 (d, |
| | 1H), 8.33 (s, 1H), 8.20 (s, 1H), 7.74 (s, 1H), 7.40 (s, 2H), 6.25 (d, 1H), 3.67 (d, |
| | 1H), 3.23 (d, 1H), 2.81 (t, 1H), 2.25 (s, 6H), 1.97 (t, 6H), 1.82 (d, 1H), 1.73- |
| | 1.65 (m, 1H), 1.61-1.49 (m, 3H). |

### Example 187 and 188. Synthesis of (R)-4-amino-N-(5-(3-aminotetrahydro-2H-pyran-3-yl)pyridin-3-yl)-1-(2,6-dichloro-4-methoxyphenyl)-6-oxo-1,6-dihydropyrimidine-5-carboxamide and (S)-4-amino-N-(5-(3-aminotetrahydro-2H-pyran-3-yl)pyridin-3-yl)-1-(2,6-dichloro-4-methoxyphenyl)-6-oxo-1,6-dihydropyrimidine-5-carboxamide

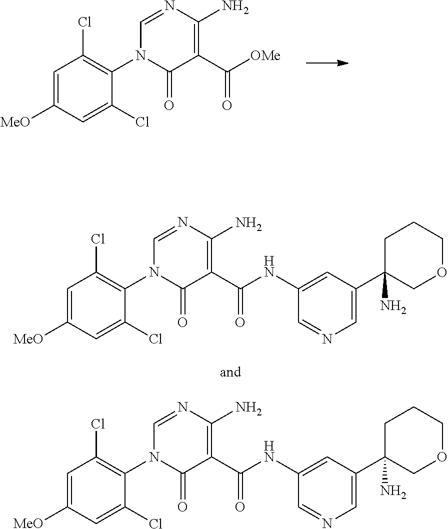 [SMILES: COC(=O)c1c(N)ncn(-c2c(Cl)cc(OC)cc2Cl)c1=O ; COc1cc(Cl)c(-n2cnc(N)c(C(=O)Nc3cncc([C@]4(N)CCCOC4)c3)c2=O)c(Cl)c1 ; COc1cc(Cl)c(-n2cnc(N)c(C(=O)Nc3cncc([C@@]4(N)CCCOC4)c3)c2=O)c(Cl)c1]

AlMe₃ (2 M, 0.26 mL) was added to a mixture of methyl 4-amino-1-(2,6-dichloro-4-methoxy-phenyl)-6-oxo-pyrimidine-5-carboxylate (Intermediate B10, 60 mg, 0.174 mmol) and 5-(3-aminotetrahydro-2H-pyran-3-yl)pyridin-3-amine (Intermediate A55, 40.4 mg, 0.209 mmol) in toluene (2 mL) at 0° C. under N₂ and the mixture stirred at 100° C. for 0.5 h under N₂. The mixture reaction was quenched by H₂O (0.2 mL) and TFA (0.2 mL) and the mixture purified by prep-HPLC-1 (5-40% MeCN). The residue further separated by SFC (DAICEL CHIRALCEL OX, 250×30 mm, 10 μm); 55% EtOH (+0.1% NH₄OH)) to afford:

Peak 1, Example 187. (R)-4-amino-N-(5-(3-aminotetrahydro-2H-pyran-3-yl)pyridin-3-yl)-1-(2,6-dichloro-4-methoxyphenyl)-6-oxo-1,6-dihydropyrimidine-5-carboxamide or (S)-4-amino-N-(5-(3-aminotetrahydro-2H-pyran-3-yl)pyridin-3-yl)-1-(2,6-dichloro-4-methoxyphenyl)-6-oxo-1,6-dihydropyrimidine-5-carboxamide. White solid (20.1 mg, 21%). LCMS m/z=505 [M+H]⁺; ¹H NMR (400 MHz, CDCl₃): 11.70 (s, 1H), 9.98 (d, 1H), 8.70 (d, 1H), 8.48 (d, 1H), 8.23 (t, 1H), 7.66 (s, 1H), 6.99 (s, 2H), 5.96 (d, 1H), 3.86-3.80 (m, 4H), 3.67 (d, 1H), 3.53-3.46 (m, 2H), 2.06 (d, 1H), 1.91-1.82 (m, 1H), 1.74-1.71 (m, 1H), 1.54-1.47 (m, 1H).

Peak 2, Example 188. R)-4-amino-N-(5-(3-aminotetrahydro-2H-pyran-3-yl)pyridin-3-yl)-1-(2,6-dichloro-4-methoxyphenyl)-6-oxo-1,6-dihydropyrimidine-5-carboxamide or (S)-4-amino-N-(5-(3-aminotetrahydro-2H-pyran-3-yl)pyridin-3-yl)-1-(2,6-dichloro-4-methoxyphenyl)-6-oxo-1,6-dihydropyrimidine-5-carboxamide. White solid (14.5 mg, 44%). %). LCMS m/z=505 [M+H]⁺; ¹H NMR (400 MHz, CDCl₃): 11.81 (s, 1H), 10.09 (d, 1H), 8.81 (d, 1H), 8.58 (d, 1H), 8.33 (t, 1H), 7.77 (s, 1H), 7.10 (s, 2H), 6.05 (d, 1H), 3.96-3.91 (m, 4H), 3.77 (d, 1H), 3.63-3.59 (m, 2H), 2.17-2.12 (m, 1H), 2.03 (s, 1H), 1.85-1.81 (m, 1H), 1.64-1.58 (m, 1H).

### Example 189 and 190. Synthesis of (R)-4-amino-1-(2,6-dichlorophenyl)-6-oxo-N-(3-(5-oxopiperazin-2-yl)phenyl)-1,6-dihydropyrimidine-5-carboxamide and (S)-4-amino-1-(2,6-dichlorophenyl)-6-oxo-N-(3-(5-oxopiperazin-2-yl)phenyl)-1,6-dihydropyrimidine-5-carboxamide

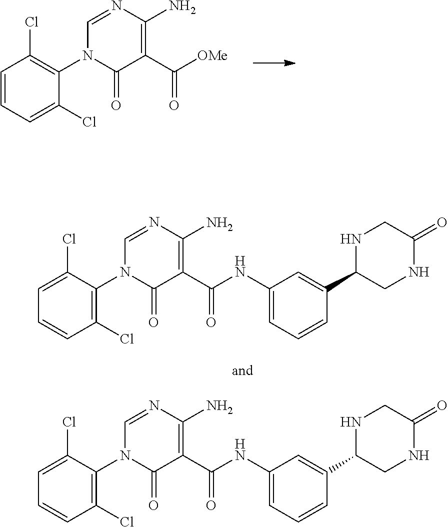 [SMILES: COC(=O)c1c(N)ncn(-c2c(Cl)cccc2Cl)c1=O ; Nc1ncn(-c2c(Cl)cccc2Cl)c(=O)c1C(=O)Nc1cccc([C@@H]2CNC(=O)CN2)c1 ; Nc1ncn(-c2c(Cl)cccc2Cl)c(=O)c1C(=O)Nc1cccc([C@H]2CNC(=O)CN2)c1]

The title compounds were prepared from methyl 4-amino-1-(2,6-dichlorophenyl)-6-oxo-1,6-dihydropyrimidine-5-carboxylate (Intermediate B9) and 5-(3-aminophenyl) piperazin-2-one (Intermediate A18) using an analogous method to that described for Example 179 and 180. SFC (DAICEL CHIRALPAK 1H-3, 50×4.6 mm, 3 μm); MeOH (+0.1% isopropylamine)) to afford:

Peak 1, Example 189. (R)-4-amino-1-(2,6-dichlorophenyl)-6-oxo-N-(3-(5-oxopiperazin-2-yl)phenyl)-1,6-dihydropyrimidine-5-carboxamide or (S)-4-amino-1-(2,6-dichlorophenyl)-6-oxo-N-(3-(5-oxopiperazin-2-yl)phenyl)-1,6-dihydropyrimidine-5-carboxamide. Yellow solid (59.5 mg, 74%). %). LCMS m/z=473 [M+H]⁺; ¹H NMR (400 MHz, DMSO-d₆): 11.78 (s, 1H), 9.74 (br d, 1H), 8.74-8.71 (m, 1H), 8.46 (s, 1H), 7.82-7.77 (m, 3H), 7.65-7.60 (m, 3H), 7.29 (t, 1H), 7.13 (br d, 1H), 3.90-3.88 (m, 1H), 3.36 (s, 3H), 3.31-3.27 (m, 2H), 3.13-3.08 (m, 1H), 2.96 (br s, 1H).

Peak 2, Example 190. (R)-4-amino-1-(2,6-dichlorophenyl)-6-oxo-N-(3-(5-oxopiperazin-2-yl)phenyl)-1,6-dihydropyrimidine-5-carboxamide or (S)-4-amino-1-(2,6-dichlorophenyl)-6-oxo-N-(3-(5-oxopiperazin-2-yl)phenyl)-1,6-dihydropyrimidine-5-carboxamide. White solid (42.8 mg, 53%). LCMS m/z=473 [M+H]⁺; ¹H NMR (400 MHz, DMSO-d₆): 11.76 (s, 1H), 9.72 (br d, 1H), 8.69 (br d, 1H), 8.43 (s, 1H), 7.78-7.74 (m, 3H), 7.66-7.57 (m, 3H), 7.27 (t, 1H), 7.10 (d, 1H), 3.88 (dd, 1H), 3.31-3.24 (m, 3H), 3.12-3.06 (m, 1H).

### Example 191. Synthesis of 4-amino-1-(2,6-dichlorophenyl)-N-(3-((2R,5R)-5-(hydroxymethyl) pyrrolidin-2-yl)phenyl)-6-oxo-1,6-dihydropyrimidine-5-carboxamide or 4-amino-1-(2,6-dichlorophenyl)-N-(3-((2S,5S)-5-(hydroxymethyl) pyrrolidin-2-yl)phenyl)-6-oxo-1,6-dihydropyrimidine-5-carboxamide

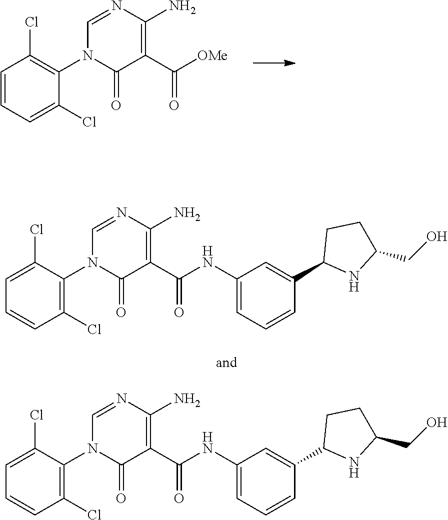

Methyl 4-amino-1-(2,6-dichlorophenyl)-6-oxo-1,6-dihydropyrimidine-5-carboxylate (Intermediate B9, 51 mg, 0.160 mmol) and tert-butyl 2-(3-aminophenyl)-5-(hydroxymethyl) pyrrolidine-1-carboxylate (Intermediate A65, 55.8 mg, 0.192 mmol) was evacuated and backfilled three time with nitrogen. Anhydrous toluene (3 mL) was added and the solution cooled to 0° C. To this was added AlMes solution (2.0 M in toluene, 0.24 mL) and the reaction stirred at RT for 10 min and then at 100° C. for 1 h. The reaction was cooled to RT and quenched with methanol (2 mL) and TFA (0.2 mL), poured into 30 mL water, and extracted with DCM (4×10 mL). The combined organics were washed with saturated aqueous sodium bicarbonate, water, brine, dried (MgSO₄), and evaporated to dryness. The residue was dissolved in DCM (2 mL) and TFA (1 mL) and the solution stirred at RT for 3 h. The reaction mixture was evaporated to dryness under reduced pressure and the residue purified by prep-HPLC-10 (10-60% MeCN) followed by chiral SFC (Rilas Technologies, Regis Whelk O-1 (S,S) 21×250 mm, 45% EtOH+0.25% diethylamine in CO₂) to afford:

Peak 1, Example 191. 4-amino-1-(2,6-dichlorophenyl)-N-(3-((2R,5R)-5-(hydroxymethyl) pyrrolidin-2-yl)phenyl)-6-oxo-1,6-dihydropyrimidine-5-carboxamide or 4-amino-1-(2,6-dichlorophenyl)-N-(3-((2S,5S)-5-(hydroxymethyl) pyrrolidin-2-yl)phenyl)-6-oxo-1,6-dihydropyrimidine-5-carboxamide. White solid (16.6 mg), LCMS m/z=474 [M+H]⁺; ¹H NMR (500 MHz, DMSO-d₆): 11.67 (s, 1H), 9.73 (d, 1H), 8.64 (d, 1H), 8.42 (s, 1H), 7.76 (d, 2H), 7.64 (t, 1H), 7.56 (d, 1H), 7.46 (s, 1H), 7.21 (t, 1H), 7.06 (d, 1H), 4.45 (s, 1H), 4.06 (t, 1H), 3.40-3.34 (m, 1H), 3.23-3.14 (m, 1H), 2.57 (q, 1H), 2.04 (dtd, 1H), 1.78 (dq, 1H), 1.58-1.37 (m, 2H), 1.03 (dt, 1H).

### Example 192. Synthesis of 4-amino-1-(2,6-dichloro-4-methoxyphenyl)-N-(5,6-dihydrospiro[cyclopenta[c]pyridine-7,2′-pyrrolidin]-4-yl)-6-oxo-1,6-dihydropyrimidine-5-carboxamide

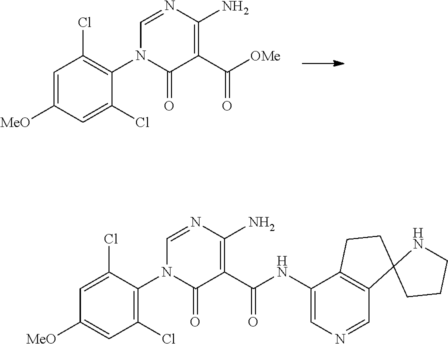 [SMILES: COC(=O)c1c(N)ncn(-c2c(Cl)cc(OC)cc2Cl)c1=O ; COc1cc(Cl)c(-n2cnc(N)c(C(=O)Nc3cncc4c3CCC43CCCN3)c2=O)c(Cl)c1]

To a solution of methyl 4-amino-1-(2,6-dichloro-4-methoxy-phenyl)-6-oxo-pyrimidine-5-carboxylate (Intermediate B10, 30 mg, 0.087 mmol) and 5,6-dihydrospiro[cyclopenta[c]pyridine-7,2′-pyrrolidin]-4-amine (Intermediate A42, 26.4 mg, 0.139 mmol) in THF (1.5 mL) was added LiHMDS (1 M, 0.262 mL) at 0° C. and the mixture was stirred at 20° C. for 12 h under N₂. The reaction mixture was quenched by addition H₂O (1 mL) at 0° C. and concentrated under reduced pressure. The residue was purified by prep-HPLC-1 (5-40% MeCN) to give the title compound as a pale yellow solid (2.3 mg, 4.8%). LCMS m/z=501 [M+H]⁺; ¹H NMR (400 MHz, CDCl₃): 11.56 (s, 1H), 10.05 (d, 1H), 9.27 (s, 1H), 8.38 (s, 1H), 7.71 (s, 1H), 7.06 (s, 2H), 5.96 (d, 1H), 3.88 (s, 3H), 3.31-3.21 (m, 1H), 3.20-3.12 (m, 1H), 3.11-3.00 (m, 1H), 2.93-2.80 (m, 1H), 2.30-2.21 (m, 2H), 2.19-2.05 (m, 3H), 2.05-1.99 (m, 2H).

### Example 193. Synthesis of (S)-4-amino-1-(2,6-dichloro-4-(difluoromethoxy)phenyl)-6-oxo-N-(3-(pyrrolidin-2-yl)phenyl)-1,6-dihydropyrimidine-5-carboxamide

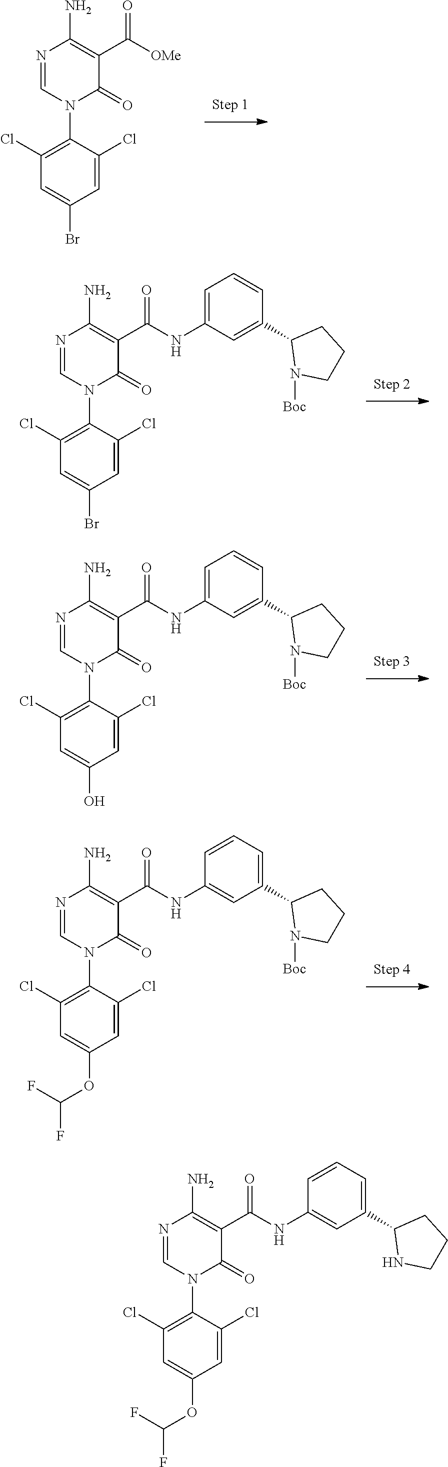

### Step 1. Synthesis of tert-butyl(S)-2-(3-(4-amino-1-(4-bromo-2,6-dichlorophenyl)-6-oxo-1,6-dihydropyrimidine-5-carboxamido)phenyl) pyrrolidine-1-carboxylate

To a solution of methyl 4-amino-1-(4-bromo-2,6-dichlorophenyl)-6-oxo-pyrimidine-5-carboxylate (Intermediate B6, 400 mg, 1.02 mmol) and tert-butyl(S)-2-(3-aminophenyl) pyrrolidine-1-carboxylate (Intermediate A61, 320 mg, 1.22 mmol) in toluene (8 mL) was added AlMe₃ (2 M in toluene, 1.53 mL) at 0° C. and the mixture was stirred at 100° C. for 1 h under N₂. The reaction mixture was quenched by addition saturated aqueous NaOH solution (5 mL) at 0° C. and extracted with EtOAc (3×7 mL). The combined organics were dried (Na₂SO₄) and concentrated under reduced pressure. The residue was purified by MPLC (SiO₂, 1-50% EtOAc/PE) to give the title compound as a white solid (180 mg, 28%). LCMS m/z=622 [M+H]⁺.

### Step 2. Synthesis of tert-butyl(S)-2-(3-(4-amino-1-(2,6-dichloro-4-hydroxyphenyl)-6-oxo-1,6-dihydropyrimidine-5-carboxamido)phenyl) pyrrolidine-1-carboxylate

To a solution of tert-butyl(S)-2-(3-(4-amino-1-(4-bromo-2,6-dichlorophenyl)-6-oxo-1,6-dihydropyrimidine-5-carboxamido)phenyl) pyrrolidine-1-carboxylate (Step 1, 150 mg, 0.241 mmol) in dioxane (3 mL) and H₂O (1 mL) was added Pd₂ (dba)₃ (22 mg, 0.024 mmol), t-Bu Xphos (20.4 mg, 0.048 mmol) and KOH (27 mg, 0.481 mmol) and the mixture was stirred at 100° C. for 1 h under N₂. The reaction mixture was concentrated under N₂ and the residue purified by prep-HPLC-16 (35-65% MeCN) to give tert-butyl(R)-2-(3-(4-amino-1-(2,6-dichloro-4-hydroxyphenyl)-6-oxo-1,6-dihydropyrimidine-5-carboxamido)phenyl) pyrrolidine-1-carboxylate or tert-butyl(S)-2-(3-(4-amino-1-(2,6-dichloro-4-hydroxyphenyl)-6-oxo-1,6-dihydropyrimidine-5-carboxamido)phenyl) pyrrolidine-1-carboxylate as a white solid (20 mg, 15%). LCMS m/z=560 [M+H]⁺.

### Step 3. Synthesis of tert-butyl(S)-2-(3-(4-amino-1-(2,6-dichloro-4-(difluoromethoxy)phenyl)-6-oxo-1,6-dihydropyrimidine-5-carboxamido)phenyl) pyrrolidine-1-carboxylate

To a solution of tert-butyl(S)-2-(3-(4-amino-1-(2,6-dichloro-4-hydroxyphenyl)-6-oxo-1,6-dihydropyrimidine-5-carboxamido)phenyl) pyrrolidine-1-carboxylate (Step 2, 15 mg, 0.027 mmol) and sodium 2-chloro-2,2-difluoroacetate (8.16 mg, 0.054 mmol) in DMF (0.5 mL) was added K₂CO₃ (4.07 mg, 0.029 mmol) and the mixture stirred at 60° C. for 30 min. The reaction mixture was concentrated under reduced pressure and the residue was diluted with H₂O (1 mL) and extracted with EtOAc (3×2 mL). The combined organic layers were washed with brine (2×1 mL), dried (Na₂SO₄) and concentrated under reduced pressure to give the title compound as a white solid (15 mg, 92%). LCMS m/z=610 [M+H]⁺.

### Step 4. Synthesis of (S)-4-amino-1-(2,6-dichloro-4-(difluoromethoxy)phenyl)-6-oxo-N-(3-(pyrrolidin-2-yl)phenyl)-1,6-dihydropyrimidine-5-carboxamide

A solution of tert-butyl(S)-2-(3-(4-amino-1-(2,6-dichloro-4-(difluoromethoxy)phenyl)-6-oxo-1,6-dihydropyrimidine-5-carboxamido)phenyl) pyrrolidine-1-carboxylate (Step 3, 15 mg, 0.025 mmol) in DCM (0.9 mL) and TFA (0.3 mL) was stirred at 25° C. for 0.5 h. The reaction mixture was concentrated under reduced pressure and the residue was purified by prep-HPLC-2 (20-55% MeCN) to give the title compound as a white solid (3 mg, 22%). LCMS m/z=510 [M+H]⁺; ¹H NMR (400 MHz, CDCl₃): 11.57 (s, 1H), 10.07 (br s, 1H), 7.76 (s, 1H), 7.69 (s, 1H), 7.46 (br d, 1H), 7.35 (s, 2H), 7.29 (br s, 1H), 7.16 (br d, 1H), 6.79-6.42 (m, 1H), 6.10 (br d, 1H), 4.42 (br d, 1H), 3.25 (br d, 2H), 2.32 (br d, 1H), 2.18-2.06 (m, 2H), 2.06-2.00 (m, 1H).

### Example 194. Synthesis of (S)-4-amino-1-(2,6-dichloro-4-cyclopropoxyphenyl)-6-oxo-N-(5-(pyrrolidin-2-yl)pyridin-3-yl)-1,6-dihydropyrimidine-5-carboxamide

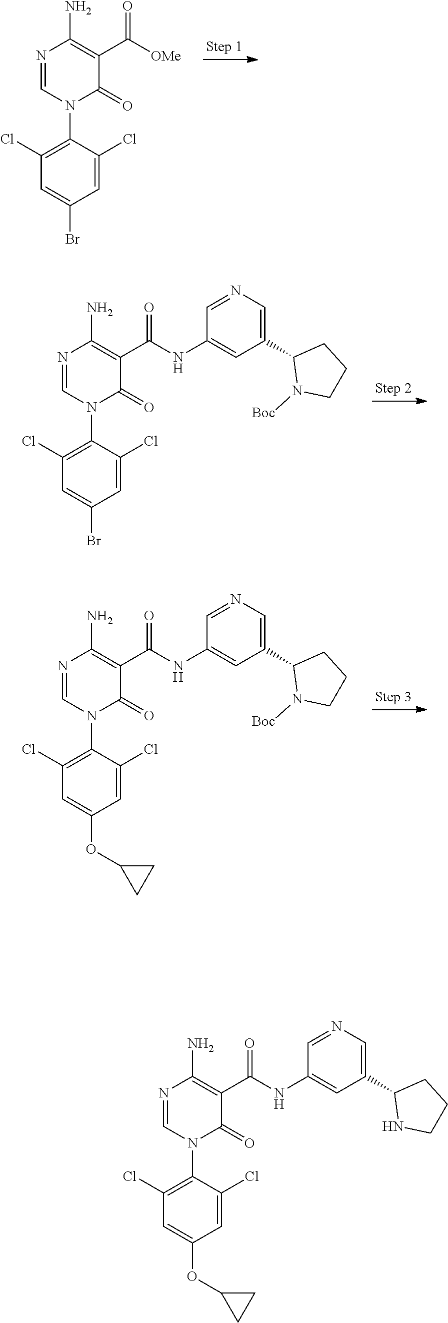 [SMILES: COC(=O)c1c(N)ncn(-c2c(Cl)cc(Br)cc2Cl)c1=O ; CC(C)(C)OC(=O)N1CCC[C@H]1c1cncc(NC(=O)c2c(N)ncn(-c3c(Cl)cc(Br)cc3Cl)c2=O)c1 ; CC(C)(C)OC(=O)N1CCC[C@H]1c1cncc(NC(=O)c2c(N)ncn(-c3c(Cl)cc(OC4CC4)cc3Cl)c2=O)c1 ; Nc1ncn(-c2c(Cl)cc(OC3CC3)cc2Cl)c(=O)c1C(=O)Nc1cncc([C@@H]2CCCN2)c1]

### Step 1. Synthesis of tert-butyl(S)-2-(5-(4-amino-1-(4-bromo-2,6-dichlorophenyl)-6-oxo-1,6-dihydropyrimidine-5-carboxamido)pyridin-3-yl) pyrrolidine-1-carboxylate

To a solution of tert-butyl(S)-2-(5-aminopyridin-3-yl) pyrrolidine-1-carboxylate (Intermediate A13, 402 mg, 1.53 mmol) and methyl 4-amino-1-(4-bromo-2,6-dichlorophenyl)-6-oxo-1,6-dihydropyrimidine-5-carboxylate (Intermediate B6, 400 mg, 1.02 mmol) in toluene (5 mL) was added AlMe₃ (2 M, 1.53 mL) at 0° C. and the mixture stirred at 100° C. for 1 h under N₂. The reaction mixture was quenched by addition saturated aqueous NaOH solution (4 mL) at 0° C. and extracted with EtOAc (3×10 mL). The combined organics were dried (Na₂SO₄) and concentrated under reduced pressure and the residue purified by MPLC (SiO₂, 1-100% EtOAc/PE) to give the title compound as a white solid (350 mg, 55%). LCMS m/z=625 [M+H]⁺.

### Step 2. Synthesis of tert-butyl(S)-2-(5-(4-amino-1-(2,6-dichloro-4-cyclopropoxyphenyl)-6-oxo-1,6-dihydropyrimidine-5-carboxamido)pyridin-3-yl) pyrrolidine-1-carboxylate

To a mixture of tert-butyl(S)-2-(5-(4-amino-1-(4-bromo-2,6-dichlorophenyl)-6-oxo-1,6-dihydropyrimidine-5-carboxamido)pyridin-3-yl) pyrrolidine-1-carboxylate (Step 1, 15 mg, 0.024 mmol) and cyclopropanol (6.98 mg, 0.120 mmol) in toluene (2 mL) was added RockPhos Pd G3 (2.01 mg, 2.40 μmol) and Cs₂CO₃ (11.74 mg, 36 μmol) and the mixture was stirred at 110° C. for 1 h under N₂. The reaction mixture was filtered and concentrated under reduced pressure to give a residue to give the title compound as a yellow oil (25 mg, crude). LCMS m/z=601 [M+H]⁺.

### Step 3. Synthesis of (S)-4-amino-1-(2,6-dichloro-4-cyclopropoxyphenyl)-6-oxo-N-(5-(pyrrolidin-2-yl)pyridin-3-yl)-1,6-dihydropyrimidine-5-carboxamide

A mixture of tert-butyl(S)-2-(5-(4-amino-1-(2,6-dichloro-4-cyclopropoxyphenyl)-6-oxo-1,6-dihydropyrimidine-5-carboxamido)pyridin-3-yl) pyrrolidine-1-carboxylate (Step 2, 25 mg, 0.042 mmol) in DCM (0.9 mL) and TFA (0.3 mL) was stirred at 25° C. for 1 h. The reaction mixture was concentrated under N₂ and the residue purified by prep-HPLC-6 (5-40% MeCN) to give the title compound as a brown oil (10.6 mg, 46%). LCMS m/z=501 [M+H]⁺; 1H NMR (400 MHz, DMSO-d₆): 11.85 (s, 1H), 9.60 (d, 1H), 8.75 (d, 2H), 8.41 (s, 1H), 8.29 (s, 1H), 8.21 (s, 1H), 8.06 (s, 1H), 7.44 (s, 2H), 4.25 (d, 1H), 4.06 (td, 1H), 3.58 (t, 1H), 3.17-3.09 (m, 1H), 3.04 (s, 1H), 2.22 (d, 1H), 1.94-1.80 (m, 2H), 1.76-1.63 (m, 1H), 0.92-0.79 (m, 2H), 0.78-0.61 (m, 2H).

### Example 195. Synthesis of (S)-4-amino-1-(2,6-dichloro-4-cyclopropoxyphenyl)-N-(5-(1-(methylamino)ethyl)pyridin-3-yl)-6-oxo-1,6-dihydropyrimidine-5-carboxamide or (R)-4-amino-1-(2,6-dichloro-4-cyclopropoxyphenyl)-N-(5-(1-(methylamino)ethyl)pyridin-3-yl)-6-oxo-1,6-dihydropyrimidine-5-carboxamide

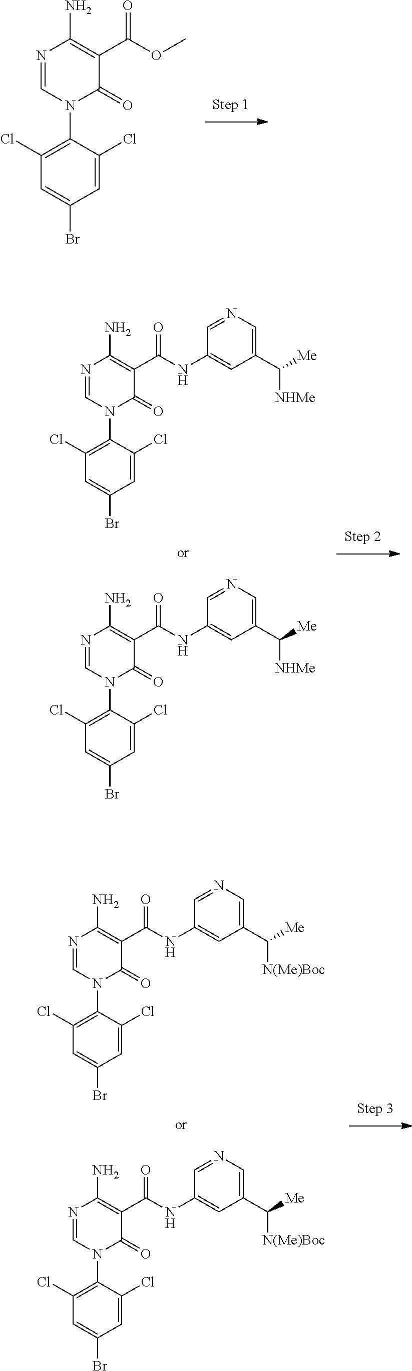

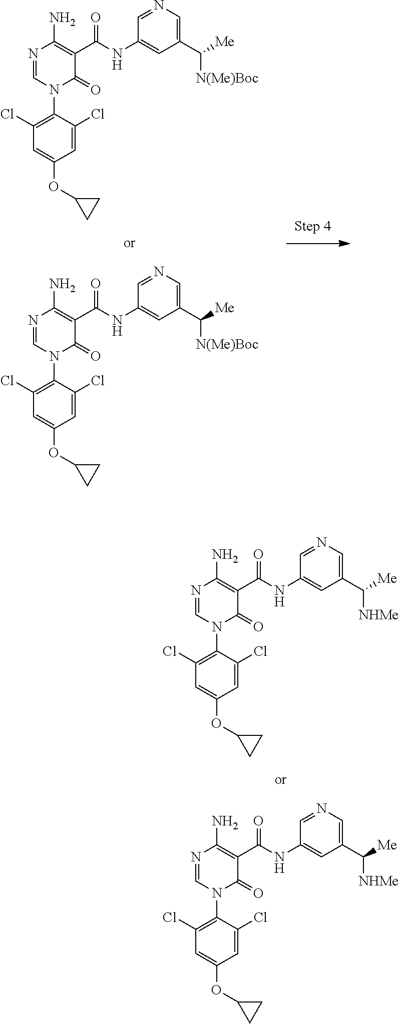

### Step 1. Synthesis of (S)-4-amino-1-(4-bromo-2,6-dichlorophenyl)-N-(5-(1-(methylamino)ethyl)pyridin-3-yl)-6-oxo-1,6-dihydropyrimidine-5-carboxamide or (R)-4-amino-1-(4-bromo-2,6-dichlorophenyl)-N-(5-(1-(methylamino)ethyl)pyridin-3-yl)-6-oxo-1,6-dihydropyrimidine-5-carboxamide

To a mixture of methyl 4-amino-1-(4-bromo-2,6-dichlorophenyl)-6-oxo-pyrimidine-5-carboxylate (Intermediate B6, 190 mg, 0.483 mmol) and (S)-5-(1-(methylamino)ethyl)pyridin-3-amine or (R)-5-(1-(methylamino)ethyl)pyridin-3-amine (Intermediate A14, 110 mg, 0.725 mmol) in toluene (13 mL) was added AlMe₃ (2 M, 0.725 mL) at 0° C. and the mixture stirred at 60° C. for 0.2 h under N₂. The reaction mixture was diluted with H₂O (5 mL) and TFA (5 mL) at 0° C. and the mixture concentrated under reduced pressure to give the title compound as a yellow solid (350 mg, crude). LCMS m/z=513 [M+H]⁺.

### Step 2. Synthesis of tert-butyl tert-butyl(S)-(1-(5-(4-amino-1-(4-bromo-2,6-dichlorophenyl)-6-oxo-1,6-dihydropyrimidine-5-carboxamido)pyridin-3-yl)ethyl)(methyl)carbamate or tert-butyl (R)-(1-(5-(4-amino-1-(4-bromo-2,6-dichlorophenyl)-6-oxo-1,6-dihydropyrimidine-5-carboxamido)pyridin-3-yl)ethyl)(methyl)carbamate

To a solution of (S)-4-amino-1-(4-bromo-2,6-dichlorophenyl)-N-(5-(1-(methylamino)ethyl)pyridin-3-yl)-6-oxo-1,6-dihydropyrimidine-5-carboxamide or (R)-4-amino-1-(4-bromo-2,6-dichlorophenyl)-N-(5-(1-(methylamino)ethyl)pyridin-3-yl)-6-oxo-1,6-dihydropyrimidine-5-carboxamide (Step 1, 350 mg, 0.683 mmol) in THF (15 mL) and H₂O (5 mL) was added NaHCO₃ (115 mg, 1.37 mmol) and di-tert-butyl dicarbonate (149 mg, 0.683 mmol) and the mixture stirred at 25° C. for 1 h. The reaction mixture was added to H₂O (50 mL) at 0° C. and extracted with EtOAc (3×20 mL). The combined organics were dried (Na₂SO₄) and concentrated under reduced pressure. The residue was purified by column chromatography (SiO₂, 0-50% EtOAc/PE) to give the title compound as a yellow solid (370 mg, 88%). LCMS m/z=613 [M+H]⁺.

### Step 3. Synthesis of tert-butyl(S)-(1-(5-(4-amino-1-(2,6-dichloro-4-cyclopropoxyphenyl)-6-oxo-1,6-dihydropyrimidine-5-carboxamido)pyridin-3-yl)ethyl)(methyl)carbamate or tert-butyl (R)-(1-(5-(4-amino-1-(2,6-dichloro-4-cyclopropoxyphenyl)-6-oxo-1,6-dihydropyrimidine-5-carboxamido)pyridin-3-yl)ethyl)(methyl)carbamate

To a solution of tert-butyl tert-butyl(S)-(1-(5-(4-amino-1-(4-bromo-2,6-dichlorophenyl)-6-oxo-1,6-dihydropyrimidine-5-carboxamido)pyridin-3-yl)ethyl)(methyl)carbamate or tert-butyl (R)-(1-(5-(4-amino-1-(4-bromo-2,6-dichlorophenyl)-6-oxo-1,6-dihydropyrimidine-5-carboxamido)pyridin-3-yl)ethyl)(methyl)carbamate (Step 2, 55 mg, 0.090 mol) in toluene (5 mL) was added RockPhos Pd G3 (7.53 mg, 8.98 μmol) and Cs₂CO₃ (43.9 mg, 0.135 mmol) and cyclopropanol (26 mg, 0.449 mmol) and the mixture stirred at 110° C. for 1.5 h under N₂. The reaction mixture was filtered and the filtrate was concentrated under reduced pressure. The residue was diluted with H₂O (30 mL) and extracted with EtOAc (3×20 mL), dried (Na₂SO₄) and concentrated under reduced pressure. The residue was purified by prep-TLC (50% EtOAc/PE) to give the title compound as a yellow solid (80 mg, 75%). LCMS m/z=589 [M+H]⁺.

### Step 4. Synthesis of (S)-4-amino-1-(2,6-dichloro-4-cyclopropoxyphenyl)-N-(5-(1-(methylamino)ethyl)pyridin-3-yl)-6-oxo-1,6-dihydropyrimidine-5-carboxamide or (S)-4-amino-1-(2,6-dichloro-4-cyclopropoxyphenyl)-N-(5-(1-(methylamino)ethyl)pyridin-3-yl)-6-oxo-1,6-dihydropyrimidine-5-carboxamide

A mixture of tert-butyl(S)-(1-(5-(4-amino-1-(2,6-dichloro-4-cyclopropoxyphenyl)-6-oxo-1,6-dihydropyrimidine-5-carboxamido)pyridin-3-yl)ethyl)(methyl)carbamate or tert-butyl (R)-(1-(5-(4-amino-1-(2,6-dichloro-4-cyclopropoxyphenyl)-6-oxo-1,6-dihydropyrimidine-5-carboxamido)pyridin-3-yl)ethyl)(methyl)carbamate (Step 3, 70 mg, 0.1189 mmol) in HCl/EtOAc (5 mL) was stirred at 25° C. for 1 h. The reaction mixture was concentrated under N₂ and the residue was purified by prep-HPLC-3 (5-40% MeCN) the title compound as a pale yellow solid (13.9 mg, 21%). LCMS m/z=489 [M+H]⁺; ¹H NMR (400 MHz, CDCl₃): 11.86 (s, 1H), 10.00 (d, 1H), 8.66 (s, 1H), 8.32 (d, 2H), 7.73 (s, 1H), 7.21 (s, 2H), 6.19 (d, 1H) 3.93 (d, 1H), 3.75-3.84 (m, 1H), 2.38 (s, 3H), 1.56 (d, 3H) 0.78-0.94 (m, 4H).

### Example 196. Synthesis of methyl(S)-4-(4-amino-6-oxo-5-((5-(piperidin-2-yl)pyridin-3-yl)carbamoyl)pyrimidin-1 (6H)-yl)-3,5-dichlorobenzoate

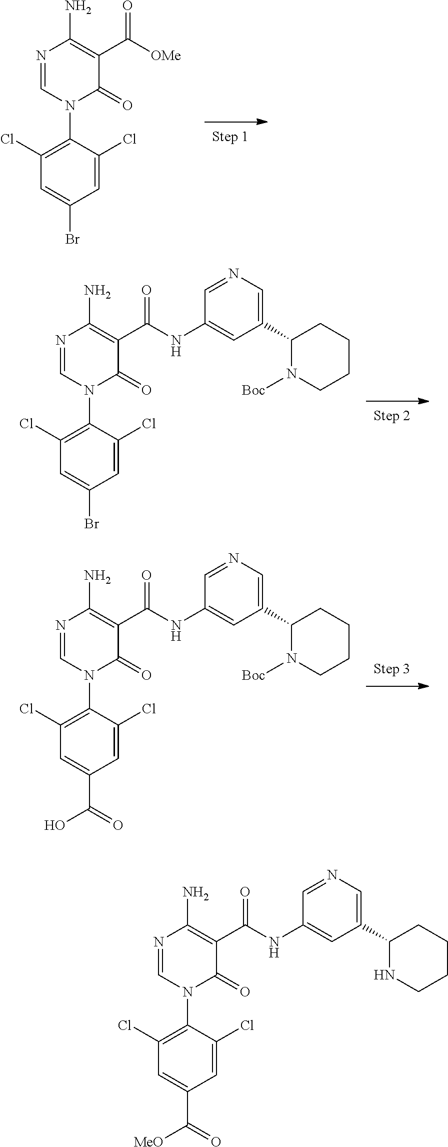 [SMILES: COC(=O)c1c(N)ncn(-c2c(Cl)cc(Br)cc2Cl)c1=O ; CC(C)(C)OC(=O)N1CCCC[C@H]1c1cncc(NC(=O)c2c(N)ncn(-c3c(Cl)cc(Br)cc3Cl)c2=O)c1 ; CC(C)(C)OC(=O)N1CCCC[C@H]1c1cncc(NC(=O)c2c(N)ncn(-c3c(Cl)cc(C(=O)O)cc3Cl)c2=O)c1 ; COC(=O)c1cc(Cl)c(-n2cnc(N)c(C(=O)Nc3cncc([C@@H]4CCCCN4)c3)c2=O)c(Cl)c1]

### Step 1. Synthesis of tert-butyl(S)-2-(5-(4-amino-1-(4-bromo-2,6-dichlorophenyl)-6-oxo-1,6-dihydropyrimidine-5-carboxamido)pyridin-3-yl) piperidine-1-carboxylate

To a solution of methyl 4-amino-1-(4-bromo-2,6-dichlorophenyl)-6-oxo-pyrimidine-5-carboxylate (100 mg, 0.254 mmol) and tert-butyl(S)-2-(5-aminopyridin-3-yl) piperidine-1-carboxylate (Intermediate A5, 70.6 mg, 0.254 mmol) in toluene (3 mL) was added AlMe₃ (2 M, 0.382 mL in toluene) at 0° C. and the mixture was stirred at 100° C. for 1 h under N₂. The reaction mixture was quenched by addition saturated aqueous NaOH (1M, 5 mL) at 0° C. and then extracted with EtOAc (3×10 mL). The combined organics were dried (Na₂SO₄) and concentrated under reduced pressure and the residue was purified by prep-TLC (50% EtOAc/PE) to give the title compound as a white solid (118 mg, 72%). LCMS m/z=639 [M+H]⁺.

### Step 2. Synthesis of (S)-4-(4-amino-5-((5-(1-(tert-butoxycarbonyl) piperidin-2-yl)pyridin-3-yl)carbamoyl)-6-oxopyrimidin-1 (6H)-yl)-3,5-dichlorobenzoic acid

To a solution of oxalic acid (116.5 mg, 1.29 mmol) in DMF (2 mL) was added tert-butyl(S)-2-(5-(4-amino-1-(4-bromo-2,6-dichlorophenyl)-6-oxo-1,6-dihydropyrimidine-5-carboxamido)pyridin-3-yl) piperidine-1-carboxylate (Step 1, 118 mg, 0.185 mmol), Pd(OAc)₂ (4.15 mg, 0.018 mmol), Ac₂O (56.6 mg, 0.555 mmol), DIPEA (71.7 mg, 0.555 mmol) and Xantphos (10.7 mg, 0.018 mmol) and the mixture was stirred at 100° C. for 12 h under N₂. The reaction mixture was concentrated under reduced pressure and the residue diluted with H₂O (5 mL) and extracted with EtOAc (3×5 mL), dried (Na₂SO₄) and concentrated under reduced pressure. The residue was purified by prep-TLC (10:1 EtOAc/MeOH) to give the title compound as a brown oil (70 mg, 63%). LCMS m/z=603 [M+H]⁺.

### Step 3. Synthesis of methyl(S)-4-(4-amino-6-oxo-5-((5-(piperidin-2-yl)pyridin-3-yl)carbamoyl)pyrimidin-1 (6H)-yl)-3,5-dichlorobenzoate

To a solution of (S)-4-(4-amino-5-((5-(1-(tert-butoxycarbonyl) piperidin-2-yl)pyridin-3-yl)carbamoyl)-6-oxopyrimidin-1 (6H)-yl)-3,5-dichlorobenzoic acid (Step 2, 70 mg, 0.116 mmol) in MeOH (1 mL) was added SOCl₂ (55.2 mg, 0.464 mmol) at 0° C. and the mixture stirred at 80° C. for 2 h under N₂. The reaction mixture was filtered and the filtrate was concentrated under reduced pressure and the residue purified by prep-HPLC-1 (3-35% MeCN) to give the title compound as a white solid (20 mg, 30%). LCMS m/z=517 [M+H]⁺; ¹H NMR (400 MHz, CDCl₃): 11.66 (s, 1H), 10.04 (d, 1H), 8.61 (d, 1H), 8.36 (s, 1H), 8.33-8.34 (m, 1H), 8.19 (s, 2H), 7.71 (s, 1H), 6.18 (d, 1H), 4.00 (s, 3H), 3.81 (m, 1H), 3.21 (d, 1H), 1.77-2.00 (m, 4H), 1.68-1.74 (m, 2H), 1.51-1.61 (m, 1H).

### Example 197. Synthesis of (R)-4-amino-N-(5-(1-amino-3-methoxypropyl)pyridin-3-yl)-1-(2,6-dichlorophenyl)-6-oxo-1,6-dihydropyrimidine-5-carboxamide or (S)-4-amino-N-(5-(1-amino-3-methoxypropyl)pyridin-3-yl)-1-(2,6-dichlorophenyl)-6-oxo-1,6-dihydropyrimidine-5-carboxamide

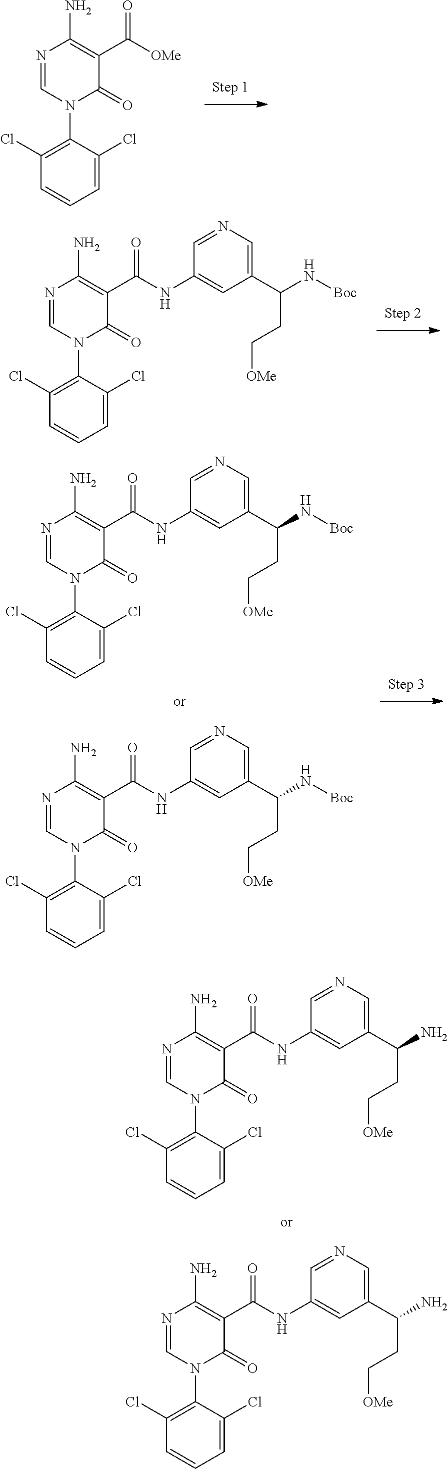

### Step 1. Synthesis of tert-butyl(1-(5-(4-amino-1-(2,6-dichlorophenyl)-6-oxo-1,6-dihydropyrimidine-5-carboxamido)pyridin-3-yl)-3-methoxypropyl)carbamate

To a mixture of methyl 4-amino-1-(2,6-dichlorophenyl)-6-oxo-1,6-dihydropyrimidine-5-carboxylate (Intermediate B9, 20 mg, 0.064 mmol) and tert-butyl(1-(5-aminopyridin-3-yl)-3-methoxypropyl)carbamate (Intermediate A19, 19.7 mg, 0.070 mmol) in toluene (3 mL) was added AlMe₃ (2M in toluene, 96 μL) at 0° C. and the mixture stirred at 0° C. for 5 min. The mixture was stirred at 40° C. for 60 min under N₂. The reaction mixture was diluted with H₂O (0.2 mL) and TFA (0.2 mL) at 0° C. and the reaction mixture was concentrated under reduced pressure. The residue was purified by prep-HPLC-2 (30-60% MeCN) to give the title compound as a white solid (30 mg, 83%). LCMS m/z=563 [M+H]⁺.

### Step 2. Synthesis of tert-butyl(S)-(1-(5-(4-amino-1-(2,6-dichlorophenyl)-6-oxo-1,6-dihydropyrimidine-5-carboxamido)pyridin-3-yl)-3-methoxypropyl)carbamate or tert-butyl (R)-(1-(5-(4-amino-1-(2,6-dichlorophenyl)-6-oxo-1,6-dihydropyrimidine-5-carboxamido)pyridin-3-yl)-3-methoxypropyl)carbamate

tert-butyl(1-(5-(4-amino-1-(2,6-dichlorophenyl)-6-oxo-1,6-dihydropyrimidine-5-carboxamido)pyridin-3-yl)-3-methoxypropyl)carbamate (Step 1, 118.7 mg, 211 mmol) was purified by SFC separation (REGIS (S,S) WHELK-01, 250×25 mm, 10 μm); 45% EtOH (0.1% NH₄OH) in CO₂) to afford:

Peak 2. tert-butyl(S)-(1-(5-(4-amino-1-(2,6-dichlorophenyl)-6-oxo-1,6-dihydropyrimidine-5-carboxamido)pyridin-3-yl)-3-methoxypropyl)carbamate or tert-butyl (R)-(1-(5-(4-amino-1-(2,6-dichlorophenyl)-6-oxo-1,6-dihydropyrimidine-5-carboxamido)pyridin-3-yl)-3-methoxypropyl)carbamate (yellow oil, 20 mg, 16%).

### Step 3. Synthesis of (R)-4-amino-N-(5-(1-amino-3-methoxypropyl)pyridin-3-yl)-1-(2,6-dichlorophenyl)-6-oxo-1,6-dihydropyrimidine-5-carboxamide or (S)-4-amino-N-(5-(1-amino-3-methoxypropyl)pyridin-3-yl)-1-(2,6-dichlorophenyl)-6-oxo-1,6-dihydropyrimidine-5-carboxamide

A mixture of tert-butyl(S)-(1-(5-(4-amino-1-(2,6-dichlorophenyl)-6-oxo-1,6-dihydropyrimidine-5-carboxamido)pyridin-3-yl)-3-methoxypropyl)carbamate or tert-butyl (R)-(1-(5-(4-amino-1-(2,6-dichlorophenyl)-6-oxo-1,6-dihydropyrimidine-5-carboxamido)pyridin-3-yl)-3-methoxypropyl)carbamate (Part 2, Peak 2, 10 mg, 0.018 mmol) in DCM (1.5 mL) and TFA (0.5 mL) was stirred at 25° C. for 0.5 h under N₂. The reaction mixture was concentrated under reduced pressure and the residue was purified by prep-HPLC-2 (5-35% MeCN) to give the title compound as a white solid (1.3 mg, 14%). LCMS m/z=463 [M+H]⁺; ¹H NMR (400 MHz, DMSO-d₆): 11.74-11.85 (m, 1H), 9.61-9.69 (m, 1H), 8.73-8.80 (m, 2H), 8.46 (s, 1H), 8.19-8.35 (m, 2H), 7.98-8.03 (m, 1H), 7.74-7.80 (m, 2H), 7.61-7.68 (m, 1H), 3.93-4.03 (m, 1H), 3.21-3.26 (m, 2H), 3.19 (s, 3H), 1.74-1.92 (m, 2H).

### Example 198. Synthesis of methyl 4-(4-amino-5-((5-((ethylamino)methyl)pyridin-3-yl)carbamoyl)-6-oxopyrimidin-1 (6H)-yl)-3,5-dichlorobenzoate

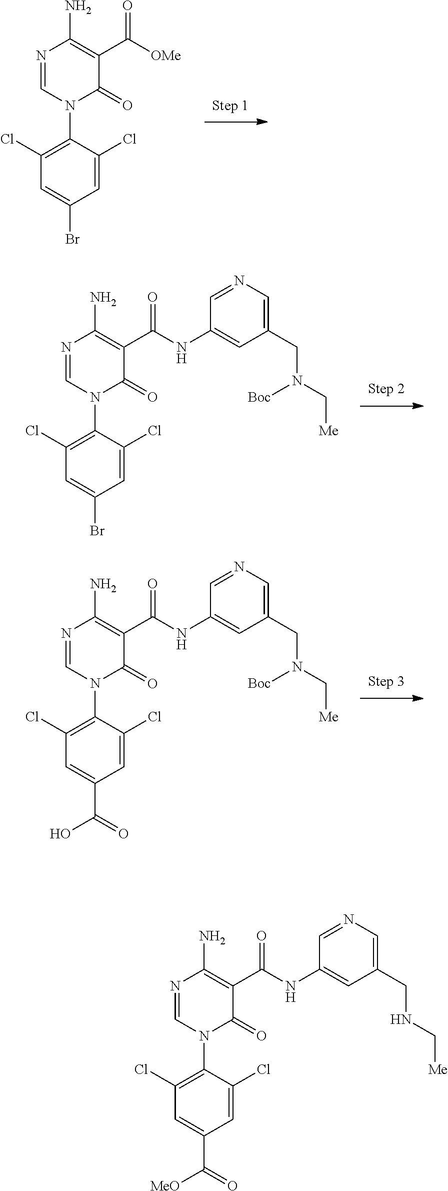 [SMILES: COC(=O)c1c(N)ncn(-c2c(Cl)cc(Br)cc2Cl)c1=O ; CCN(Cc1cncc(NC(=O)c2c(N)ncn(-c3c(Cl)cc(Br)cc3Cl)c2=O)c1)C(=O)OC(C)(C)C ; CCN(Cc1cncc(NC(=O)c2c(N)ncn(-c3c(Cl)cc(C(=O)O)cc3Cl)c2=O)c1)C(=O)OC(C)(C)C ; CCNCc1cncc(NC(=O)c2c(N)ncn(-c3c(Cl)cc(C(=O)OC)cc3Cl)c2=O)c1]

### Step 1. Synthesis of tert-butyl ((5-(4-amino-1-(4-bromo-2,6-dichlorophenyl)-6-oxo-1,6-dihydropyrimidine-5-carboxamido)pyridin-3-yl)methyl)(ethyl)carbamate

AlMe₃ (2M in toluene, 1.91 mL) was added to a mixture of methyl 4-amino-1-(4-bromo-2,6-dichlorophenyl)-6-oxo-1,6-dihydropyrimidine-5-carboxylate (Intermediate B6, 500 mg, 1.27 mmol) and tert-butyl ((5-aminopyridin-3-yl)methyl)(ethyl)carbamate (Intermediate A24, 480 mg, 1.91 mmol) in toluene (9 mL) at 0° C. and the mixture stirred at 100° C. for 0.5 h under N₂. The reaction mixture was quenched by addition 1M NaOH aqueous solution (50 mL) at 0° C. and then extracted with EtOAc (3×50 mL). The combined organics were dried (Na₂SO₄), concentrated under reduced pressure and the residue purified by column chromatography (SiO₂, 0-100% EtOAc/PE) to give the title compound as a yellow solid (2 g, 64%). LCMS m/z=613 [M+H]⁺.

### Step 2. Synthesis of 4-(4-amino-5-((5-(((tert-butoxycarbonyl) (ethyl)amino)methyl)pyridin-3-yl)carbamoyl)-6-oxopyrimidin-1 (6H)-yl)-3,5-dichlorobenzoic acid

To a solution of tert-butyl ((5-(4-amino-1-(4-bromo-2,6-dichlorophenyl)-6-oxo-1,6-dihydropyrimidine-5-carboxamido)pyridin-3-yl)methyl)(ethyl)carbamate (Step 1, 310 mg, 0.506 mmol) and oxalic acid (319 mg, 3.54 mmol) in DMF (2 mL) was added Pd(OAc)₂ (11.4 mg, 0.051 mmol), Xantphos (29.3 mg, 0.051 mmol), Ac₂O (155 mg, 1.52 mmol) and DIPEA (196 mg, 1.52 mmol) and the mixture was stirred at 100° C. for 12 h under N₂. The reaction mixture was filtered and the filtrate concentrated under reduced pressure and the residue purified by prep-HPLC-1 (30-60% MeCN) to give the title compound as a white solid (120 mg, 41%). LCMS m/z=577 [M+H]⁺.

### Step 3. Synthesis of methyl 4-(4-amino-5-((5-((ethylamino)methyl)pyridin-3-yl)carbamoyl)-6-oxopyrimidin-1 (6H)-yl)-3,5-dichlorobenzoate

To a solution of 4-(4-amino-5-((5-(((tert-butoxycarbonyl) (ethyl)amino)methyl)pyridin-3-yl)carbamoyl)-6-oxopyrimidin-1 (6H)-yl)-3,5-dichlorobenzoic acid (Part 2, 60 mg, 0.104 mmol) in MeOH (1 mL) was added thionyl chloride (49.5 mg, 0.416 mmol) at 0° C. and the mixture stirred at 80° C. for 2 h under N₂. The reaction mixture was filtered and the filtrate was concentrated under reduced pressure and the residue purified by prep-HPLC-1 (3-35% MeCN) to give the title compound as a white solid (22.6 mg, 44%). LCMS m/z=491 [M+H]⁺; ¹H NMR (400 MHz, CDCl₃): 11.59 (s, 1H), 9.97 (d, 1H), 8.53 (d, 1H), 8.32-8.18 (m, 2H), 8.11 (s, 2H), 7.63 (s, 1H), 6.19 (d, 1H), 3.92 (s, 3H), 3.83 (s, 2H), 2.70 (q, 2H), 1.10 (t, 3H).

### Example 199. Synthesis of ethyl 4-(4-amino-5-((5-((ethylamino)methyl)pyridin-3-yl)carbamoyl)-6-oxopyrimidin-1 (6H)-yl)-3,5-dichlorobenzoate

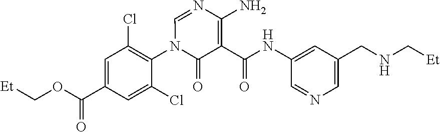 [SMILES: CCCNCc1cncc(NC(=O)c2c(N)ncn(-c3c(Cl)cc(C(=O)OCCC)cc3Cl)c2=O)c1]

The title compound was prepared as a white solid from methyl 4-amino-1-(4-bromo-2,6-dichlorophenyl)-6-oxo-1,6-dihydropyrimidine-5-carboxylate (Intermediate B6) using an analogous 3-step method to that described for Example 198. LCMS m/z=505 [M+H]⁺; 1H NMR (400 MHz, CDCl₃): 11.70 (s, 1H), 10.08 (d, 1H), 8.65 (s, 1H), 8.32 (d, 2H), 8.22 (s, 2H), 7.74 (s, 1H), 6.22 (s, 1H), 4.48 (q, 2H), 3.92 (s, 2H), 2.78 (q, 2H), 1.47 (t, 3H), 1.20 (t, 3H).

### Example 200. Synthesis of 4-amino-1-(2,6-dichloro-4-(oxazol-5-yl)phenyl)-N-(5-((ethylamino)methyl)pyridin-3-yl)-6-oxo-1,6-dihydropyrimidine-5-carboxamide

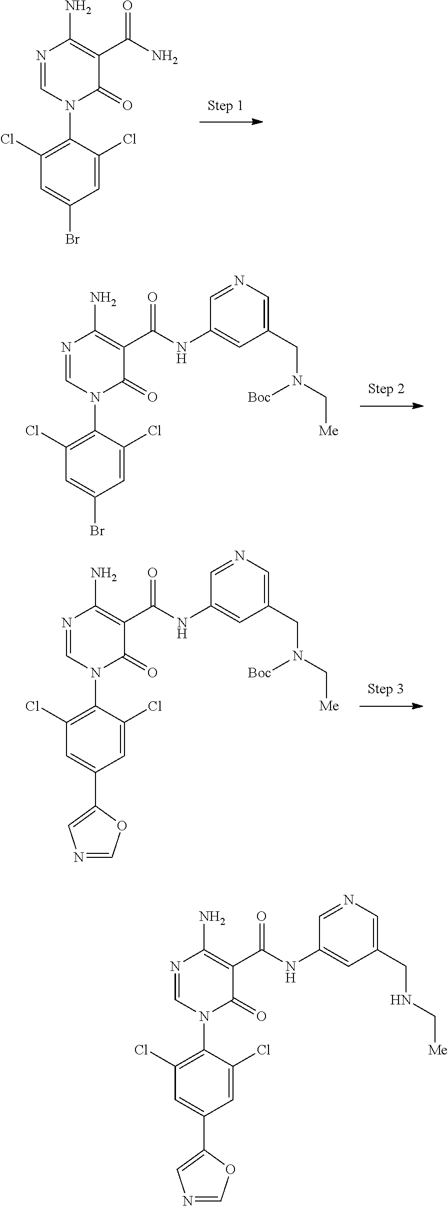

### Step 1. Synthesis of tert-butyl ((5-(4-amino-1-(4-bromo-2,6-dichlorophenyl)-6-oxo-1,6-dihydropyrimidine-5-carboxamido)pyridin-3-yl)methyl)(ethyl)carbamate

AlMe₃ (2M in toluene, 1.91 mL) was added to a mixture of methyl 4-amino-1-(4-bromo-2,6-dichlorophenyl)-6-oxo-1,6-dihydropyrimidine-5-carboxylate (Intermediate B6, 500 mg, 1.27 mmol) and tert-butyl ((5-aminopyridin-3-yl)methyl)(ethyl)carbamate (Intermediate A24, 480 mg, 1.91 mmol) in toluene (9 mL) at 0° C. and the mixture stirred at 100° C. for 0.5 h under N₂. The reaction mixture was quenched by addition 1M NaOH aqueous solution (50 mL) at 0° C. and then extracted with EtOAc (3×50 mL). The combined organics were dried (Na₂SO₄), concentrated under reduced pressure and the residue purified by column chromatography (SiO₂, 0-100% EtOAc/PE) to give the title compound as a yellow solid (2 g, 64%). LCMS m/z=613 [M+H]⁺.

### Step 2. Synthesis of tert-butyl ((5-(4-amino-1-(2,6-dichloro-4-(oxazol-5-yl)phenyl)-6-oxo-1,6-dihydropyrimidine-5-carboxamido)pyridin-3-yl)methyl)(ethyl)carbamate

A mixture of tert-butyl ((5-(4-amino-1-(4-bromo-2,6-dichlorophenyl)-6-oxo-1,6-dihydropyrimidine-5-carboxamido)pyridin-3-yl)methyl)(ethyl)carbamate (Part 1, 90 mg, 0.147 mmol), 5-(4,4,5,5-tetramethyl-1,3,2-dioxaborolan-2-yl) oxazole (57.33 mg, 0.294 mmol), K₃PO₄ (62.4 mg, 0.294 mmol), [2-(2-aminophenyl)phenyl]-chloro-palladium; dicyclohexyl-[3-(2,4,6-triisopropylphenyl)phenyl]phosphane (23.13 mg, 0.029 mmol) in dioxane (4 mL) and H₂O (0.4 mL) was degassed and purged with N₂ (×3) and the mixture stirred at 110° C. for 1 h under N₂. The reaction mixture was filtered and the filtrate was concentrated under reduced pressure to give the title compound as a white solid (80 mg, 90%). LCMS m/z=600 [M+H]⁺.

### Step 3. Synthesis of 4-amino-1-(2,6-dichloro-4-(oxazol-5-yl)phenyl)-N-(5-((ethylamino)methyl)pyridin-3-yl)-6-oxo-1,6-dihydropyrimidine-5-carboxamide

To the mixture of tert-butyl ((5-(4-amino-1-(2,6-dichloro-4-(oxazol-5-yl)phenyl)-6-oxo-1,6-dihydropyrimidine-5-carboxamido)pyridin-3-yl)methyl)(ethyl)carbamate (Part 2, 60 mg, 0.100 mmol) in DCM (2 mL) and TFA (1 mL) and the mixture was stirred at 25° C. for 30 min. The reaction mixture was concentrated under reduced pressure to give a residue which was purified by prep-HPLC-1 (20-50% MeCN) to give the title compound as a yellow oil (8.2 mg, 16%). LCMS m/z=500 [M+H]⁺; ¹H NMR (400 MHz, DMSO-d₆): 1.05 (t, 3H) 2.57 (d, 2H) 3.74 (d, 2H) 8.05 (s, 1H), 8.08 (s, 1H), 8.15 (s, 2H), 8.25 (s, 1H), 8.50 (s, 1H), 8.65 (s, 1H), 8.70 (br s, 1H), 8.85 (d, 1H), 9.69 (d, 1H), 11.78 (s, 1H).

### Example 201. Synthesis of 4-amino-1-(2,6-dichloro-4-(oxazol-2-yl)phenyl)-N-(5-((ethylamino)methyl)pyridin-3-yl)-6-oxo-1,6-dihydropyrimidine-5-carboxamide

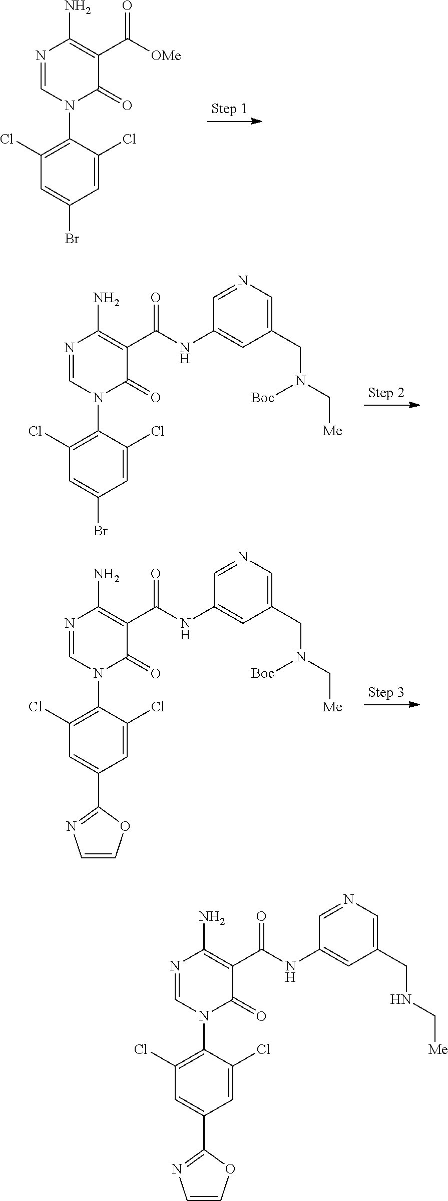

### Step 1. Synthesis of tert-butyl ((5-(4-amino-1-(4-bromo-2,6-dichlorophenyl)-6-oxo-1,6-dihydropyrimidine-5-carboxamido)pyridin-3-yl)methyl)(ethyl)carbamate

The title compound was prepared methyl 4-amino-1-(4-bromo-2,6-dichlorophenyl)-6-oxo-1,6-dihydropyrimidine-5-carboxylate (Intermediate B6) and tert-butyl ((5-aminopyridin-3-yl)methyl)(ethyl)carbamate (Intermediate A24) using an analogous method to that described for Example 190, Step 1.

### Step 2. Synthesis of tert-butyl ((5-(4-amino-1-(2,6-dichloro-4-(oxazol-2-yl)phenyl)-6-oxo-1,6-dihydropyrimidine-5-carboxamido)pyridin-3-yl)methyl)(ethyl)carbamate

To a solution of tert-butyl ((5-(4-amino-1-(4-bromo-2,6-dichlorophenyl)-6-oxo-1,6-dihydropyrimidine-5-carboxamido)pyridin-3-yl)methyl)(ethyl)carbamate (Step 1, 50 mg, 0.082 mmol) in dioxane (1.5 mL) was added cataCXium® A Pd G2 (10.92 mg, 0.016 mmol) and 2-(tributylstannyl) oxazole (117 mg, 0.327 mmol) and the mixture stirred at 80° C. for 1 h under N₂. The reaction mixture was diluted with H₂O (10 mL) and extracted with EtOAc (3×5 mL). The combined organics were washed with brine (2×1 mL), dried (Na₂SO₄) and concentrated under reduced pressure. The residue was purified by prep-HPLC-8 (30-65% MeCN) to give the title compound as a yellow solid (20 mg, 41%). LCMS m/z=600 [M+H]⁺.

### Step 3. Synthesis of 4-amino-1-(2,6-dichloro-4-(oxazol-2-yl)phenyl)-N-(5-((ethylamino)methyl)pyridin-3-yl)-6-oxo-1,6-dihydropyrimidine-5-carboxamide

The title compound was prepared from tert-butyl ((5-(4-amino-1-(2,6-dichloro-4-(oxazol-2-yl)phenyl)-6-oxo-1,6-dihydropyrimidine-5-carboxamido)pyridin-3-yl)methyl)(ethyl)carbamate (Step 2) using an analogous method to that described for Example 200, Step 3. Prep-HPLC-1 (1-30% MeCN) to give the title compound as a white solid (5.1 mg, 14%). LCMS m/z=500 [M+H]⁺; ¹H NMR (400 MHz, CDCl₃): 11.69 (s, 1H), 10.07-10.15 (m, 1H), 8.68 (d, 1H), 8.30 (s, 1H), 8.24 (s, 2H), 8.15 (s, 1H), 7.84 (s, 1H), 7.77 (s, 1H), 7.36 (s, 1H), 6.06 (br dd, 1H), 3.83 (s, 2H), 2.70 (q, 2H), 1.15 (t, 3H).

### Example 202. Synthesis of 4-amino-1-(2,6-dichloro-4-(2,2,2-trifluoroethyl)phenyl)-N-(5-((ethylamino)methyl)pyridin-3-yl)-6-oxo-1,6-dihydropyrimidine-5-carboxamide

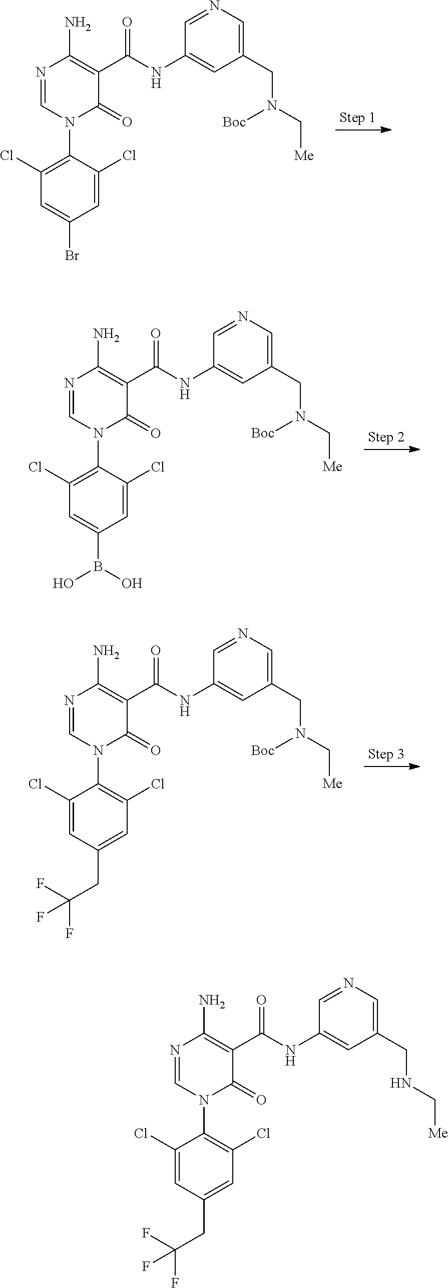 [SMILES: CCN(Cc1cncc(NC(=O)c2c(N)ncn(-c3c(Cl)cc(Br)cc3Cl)c2=O)c1)C(=O)OC(C)(C)C ; CCN(Cc1cncc(NC(=O)c2c(N)ncn(-c3c(Cl)cc(B(O)O)cc3Cl)c2=O)c1)C(=O)OC(C)(C)C ; CCN(Cc1cncc(NC(=O)c2c(N)ncn(-c3c(Cl)cc(CC(F)(F)F)cc3Cl)c2=O)c1)C(=O)OC(C)(C)C ; CCNCc1cncc(NC(=O)c2c(N)ncn(-c3c(Cl)cc(CC(F)(F)F)cc3Cl)c2=O)c1]

### Step 1. Synthesis of (4-(4-amino-5-((5-(((tert-butoxycarbonyl) (ethyl)amino)methyl)pyridin-3-yl)carbamoyl)-6-oxopyrimidin-1 (6H)-yl)-3,5-dichlorophenyl) boronic acid

To a solution of tert-butyl ((5-(4-amino-1-(4-bromo-2,6-dichlorophenyl)-6-oxo-1,6-dihydropyrimidine-5-carboxamido)pyridin-3-yl)methyl)(ethyl)carbamate (Example 190, Step 1; 315 mg, 0.514 mmol) in dioxane (5.5 mL) was added Pd(dppf) C₁₂·DCM (42 mg, 0.051 mmol) and KOAc (101 mg, 1.03 mmol) and 4,4,4′,4′,5,5,5′,5′-octamethyl-2,2′-bi (1,3,2-dioxaborolane) (653 mg, 2.57 mmol) and the mixture stirred at 80° C. for 15 h under N₂. The reaction mixture was filtered and concentrated under reduced pressure and the residue was purified by prep-HPLC-8 (15-45% MeCN) to give the title compound as a white solid (1.3 g, 73%). LCMS m/z=577 [M+H]⁺.

### Step 2. Synthesis of tert-butyl ((5-(4-amino-1-(2,6-dichloro-4-(2,2,2-trifluoroethyl)phenyl)-6-oxo-1,6-dihydropyrimidine-5-carboxamido)pyridin-3-yl)methyl)(ethyl)carbamate

A mixture of (4-(4-amino-5-((5-(((tert-butoxycarbonyl) (ethyl)amino)methyl)pyridin-3-yl)carbamoyl)-6-oxopyrimidin-1 (6H)-yl)-3,5-dichlorophenyl) boronic acid (Part 1, 15 mg, 0.026 mmol), 1,1,1-trifluoro-2-iodoethane (109 mg, 0.520 mmol), Pd₂ (dba)₃ (2.38 mg, 2.60 μmol), Xantphos (1.50 mg, 2.60 μmol) and Cs₂CO₃ (33.9 mg, 0.104 mmol) in dioxane (2 mL) and H₂O (0.1 mL) was degassed and purged with N₂ (3×) and the mixture stirred at 80° C. for 2 h under N₂. The reaction mixture was concentrated under reduced pressure to give the title compound as a white solid (45 mg, 93%) which was without further purification. LCMS m/z=615 [M+H]⁺.

### Step 3. Synthesis of 4-amino-1-(2,6-dichloro-4-(2,2,2-trifluoroethyl)phenyl)-N-(5-((ethylamino)methyl)pyridin-3-yl)-6-oxo-1,6-dihydropyrimidine-5-carboxamide

The title compound was prepared as a white solid (5.1 mg, 13%) from tert-butyl ((5-(4-amino-1-(2,6-dichloro-4-(2,2,2-trifluoroethyl)phenyl)-6-oxo-1,6-dihydropyrimidine-5-carboxamido)pyridin-3-yl)methyl)(ethyl)carbamate (Step 2, 45 mg, 0.073 mmol) using an analogous method to that described for Example 200, Step 3. LCMS m/z=515 [M+H]⁺; 1H NMR (400 MHz, CDCl₃): 1.15-1.21 (m, 3H), 2.91 (q, 2H), 3.37 (q, 2H), 4.00 (s, 2H), 6.09 (d, 1H), 7.42 (s, 2H), 7.64 (s, 1H), 8.29 (s, 1H), 8.48 (d, 2H), 9.84 (d, 2H), 11.71 (s, 1H).

### Example 203. Synthesis of 4-amino-1-(2,6-dichloro-4-(2,2-difluoroethyl)phenyl)-N-(5-((ethylamino)methyl)pyridin-3-yl)-6-oxo-1,6-dihydropyrimidine-5-carboxamide

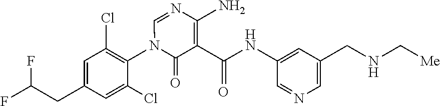 [SMILES: CCNCc1cncc(NC(=O)c2c(N)ncn(-c3c(Cl)cc(CC(F)F)cc3Cl)c2=O)c1]

The title compound was prepared as a white solid from tert-butyl ((5-(4-amino-1-(4-bromo-2,6-dichlorophenyl)-6-oxo-1,6-dihydropyrimidine-5-carboxamido)pyridin-3-yl)methyl)(ethyl)carbamate (Example 190, Step 1) and 1,1-difluoro-2-iodoethane using an analogous 3-Step method as described for Example 202. LCMS m/z=497 [M+H]⁺; 1H NMR (400 MHz, CDCl₃): 1.14-1.24 (m, 1H), 1.19 (d, 2H), 2.04 (d, 1H), 2.75 (d, 2H), 3.17-3.31 (m, 2H), 3.88 (s, 2H), 5.86-6.25 (m, 1H), 6.07-6.15 (m, 1H), 7.48 (s, 2H), 7.74 (d, 1H), 8.16-8.37 (m, 1H), 8.25 (s, 1H), 8.33 (s, 1H), 8.66 (s, 1H), 10.06 (d, 1H), 11.73 (s, 1H).

### Example 204. Synthesis of (R)-4-amino-N-(5-(1-amino-2,2-difluoroethyl)pyridin-3-yl)-1-(2,6-dichloro-4-methoxyphenyl)-6-oxo-1,6-dihydropyrimidine-5-carboxamide or (S)-4-amino-N-(5-(1-amino-2,2-difluoroethyl)pyridin-3-yl)-1-(2,6-dichloro-4-methoxyphenyl)-6-oxo-1,6-dihydropyrimidine-5-carboxamide

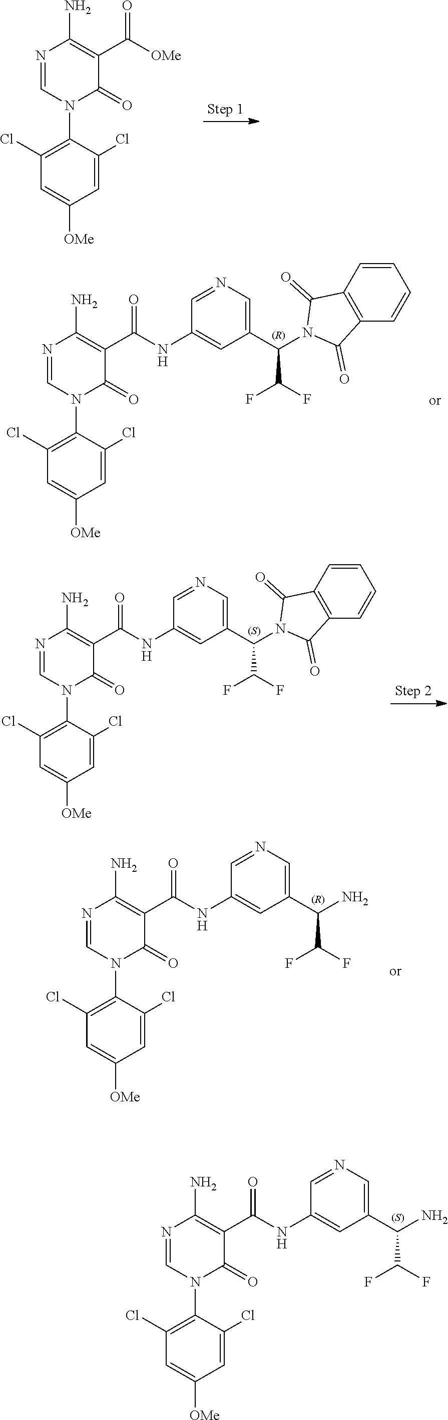

### Step 1. Synthesis of (R)-4-amino-1-(2,6-dichloro-4-methoxyphenyl)-N-(5-(1-(1,3-dioxoisoindolin-2-yl)-2,2-difluoroethyl)pyridin-3-yl)-6-oxo-1,6-dihydropyrimidine-5-carboxamide or (S)-4-amino-1-(2,6-dichloro-4-methoxyphenyl)-N-(5-(1-(1,3-dioxoisoindolin-2-yl)-2,2-difluoroethyl)pyridin-3-yl)-6-oxo-1,6-dihydropyrimidine-5-carboxamide

The title compound was prepared as a white solid (50 mg, 56%) from methyl 4-amino-1-(2,6-dichloro-4-methoxyphenyl)-6-oxo-1,6-dihydropyrimidine-5-carboxylate (Intermediate B10) and (R)-2-(1-(5-aminopyridin-3-yl)-2,2-difluoroethyl) isoindoline-1,3-dione or (S)-2-(1-(5-aminopyridin-3-yl)-2,2-difluoroethyl) isoindoline-1,3-dione (Intermediate A48) using an analogous method to that described for Example 2. LCMS m/z=615 [M+H]⁺.

### Step 2. Synthesis of (R)-4-amino-N-(5-(1-amino-2,2-difluoroethyl)pyridin-3-yl)-1-(2,6-dichloro-4-methoxyphenyl)-6-oxo-1,6-dihydropyrimidine-5-carboxamide or (S)-4-amino-N-(5-(1-amino-2,2-difluoroethyl)pyridin-3-yl)-1-(2,6-dichloro-4-methoxyphenyl)-6-oxo-1,6-dihydropyrimidine-5-carboxamide

To a solution of (R)-4-amino-1-(2,6-dichloro-4-methoxyphenyl)-N-(5-(1-(1,3-dioxoisoindolin-2-yl)-2,2-difluoroethyl)pyridin-3-yl)-6-oxo-1,6-dihydropyrimidine-5-carboxamide or (S)-4-amino-1-(2,6-dichloro-4-methoxyphenyl)-N-(5-(1-(1,3-dioxoisoindolin-2-yl)-2,2-difluoroethyl)pyridin-3-yl)-6-oxo-1,6-dihydropyrimidine-5-carboxamide (Step 1, 40 mg, 0.065 mmol) in EtOH (2 mL) was added hydrazine hydrate (15.31 mg, 0.260 mmol) and the mixture stirred at 70° C. for 1 h. The reaction mixture was filtered and the filtrate was concentrated under reduced pressure and the residue was diluted with H₂O (3 mL) and extracted with EtOAc (3×3 mL). The combined organics were washed with brine (2×2 mL), dried (Na₂SO₄) and concentrated under reduced pressure and the residue purified by prep-HPLC-3 (1-35% MeCN) to give the title compound as a brown solid (17.9 mg, 51%). LCMS m/z=485 [M+H]⁺; ¹H NMR (400 MHz, CDCl₃): 11.86 (s, 1H), 10.01 (br d, 1H), 8.75 (d, 1H), 8.34 (d, 1H), 8.27 (t, 1H), 7.74 (s, 1H), 7.07 (s, 2H), 6.03 (br d, 1H), 5.95-5.65 (m, 1H), 4.24 (ddd, 1H), 3.88 (s, 3H).

### Example 205. Synthesis of (R)-4-amino-1-(4-methoxy-2,6-dimethylphenyl)-6-oxo-N-(5-(piperidin-2-yl)pyridin-3-yl)-1,6-dihydropyrimidine-5-carboxamide

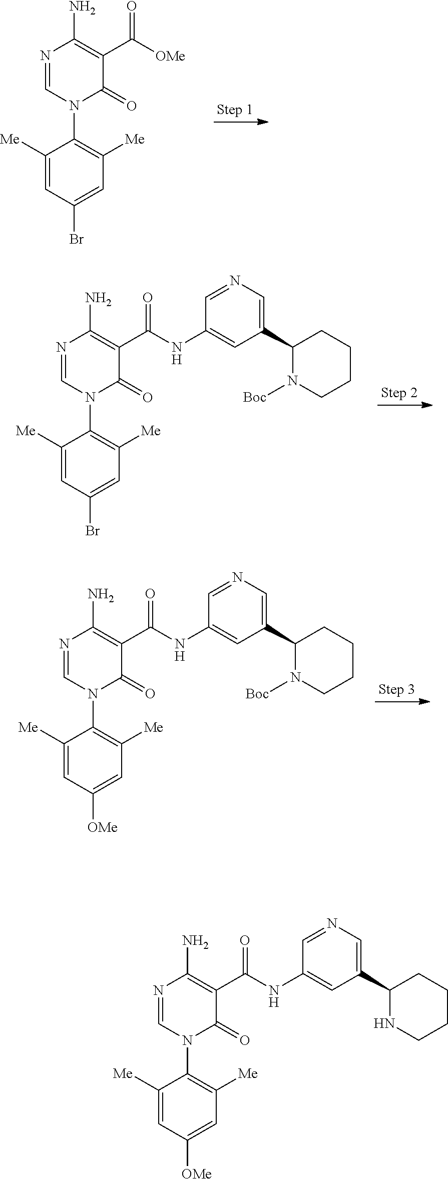 [SMILES: COC(=O)c1c(N)ncn(-c2c(C)cc(Br)cc2C)c1=O ; Cc1cc(Br)cc(C)c1-n1cnc(N)c(C(=O)Nc2cncc([C@H]3CCCCN3C(=O)OC(C)(C)C)c2)c1=O ; COc1cc(C)c(-n2cnc(N)c(C(=O)Nc3cncc([C@H]4CCCCN4C(=O)OC(C)(C)C)c3)c2=O)c(C)c1 ; COc1cc(C)c(-n2cnc(N)c(C(=O)Nc3cncc([C@H]4CCCCN4)c3)c2=O)c(C)c1]

### Step 1. Synthesis of tert-butyl(R)-2-(5-(4-amino-1-(4-bromo-2,6-dimethylphenyl)-6-oxo-1,6-dihydropyrimidine-5-carboxamido)pyridin-3-yl) piperidine-1-carboxylate

The title compound was prepared from methyl 4-amino-1-(4-bromo-2,6-dimethylphenyl)-6-oxo-1,6-dihydropyrimidine-5-carboxylate (Intermediate B34) and tert-butyl(R)-2-(5-aminopyridin-3-yl) piperidine-1-carboxylate (Intermediate A4) using an analogous method to that described for Example 2. LCMS m/z=599 [M+H]⁺.

Step 2. Synthesis of tert-butyl(R)-2-(5-(4-amino-1-(4-methoxy-2,6-dimethylphenyl)-6-oxo-1,6-dihydropyrimidine-5-carboxamido)pyridin-3-yl) piperidine-1-carboxylate. To the mixture of tert-butyl(R)-2-(5-(4-amino-1-(4-bromo-2,6-dimethylphenyl)-6-oxo-1,6-dihydropyrimidine-5-carboxamido)pyridin-3-yl) piperidine-1-carboxylate (Step 1, 250 mg, 0.418 mmol) in toluene (2 mL) and MeOH (2 mL) was added K₃PO₄ (133 mg, 0.628 mmol), t-Bu Xphos (17.77 mg, 0.042 mmol) and Pd₂ (dba)₃ (38.31 mg, 0.042 mmol) and the mixture stirred at 80° C. for 2 h under N₂. The mixture was filtered and concentrated under reduced pressure to give the title compound as a brown oil (500 mg). LCMS m/z=594 [M+H]⁺.

### Step 3. Synthesis of (R)-4-amino-1-(4-methoxy-2,6-dimethylphenyl)-6-oxo-N-(5-(piperidin-2-yl)pyridin-3-yl)-1,6-dihydropyrimidine-5-carboxamide

To the mixture of tert-butyl(R)-2-(5-(4-amino-1-(4-methoxy-2,6-dimethylphenyl)-6-oxo-1,6-dihydropyrimidine-5-carboxamido)pyridin-3-yl) piperidine-1-carboxylate (Step 2, 500 mg, 0.911 mmol) in DCM (4 mL) was added TFA (1.5 mL), the mixture was stirred at 20° C. for 0.5 h. The reaction mixture was concentrated under reduced pressure and the residue was purified by prep-HPLC-3 (5-45% MeCN) to give the title compound as a pale yellow solid (40.3 mg, 10%). LCMS m/z=449 [M+H]⁺; ¹H NMR (400 MHz, CDCl₃): 12.09 (s, 1H), 9.93 (br d, 1H), 8.64 (s, 1H), 8.39 (s, 2H), 7.79 (s, 1H), 6.78 (s, 2H), 6.03 (br d, 1H), 3.87 (s, 3H), 3.83 (br d, 1H), 3.22 (br d, 1H), 2.86-2.79 (m, 1H), 2.18 (s, 6H), 1.99-1.85 (m, 3H), 1.73 (br d, 2H), 1.62-1.54 (m, 1H).

### Example 206. Synthesis of 4-amino-N-((1R,3S)-3-(aminomethyl)cyclohexyl)-1-((S)-2-chloro-4-methoxy-6-methylphenyl)-6-oxo-1,6-dihydropyrimidine-5-carboxamide or 4-amino-N-((1S,3R)-3-(aminomethyl)cyclohexyl)-1-((S)-2-chloro-4-methoxy-6-methylphenyl)-6-oxo-1,6-dihydropyrimidine-5-carboxamide

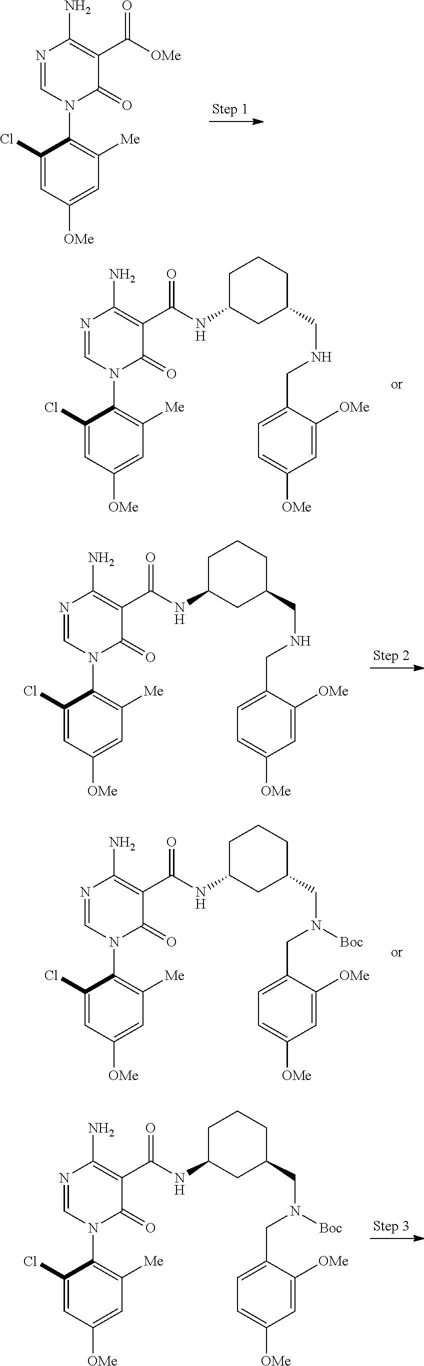

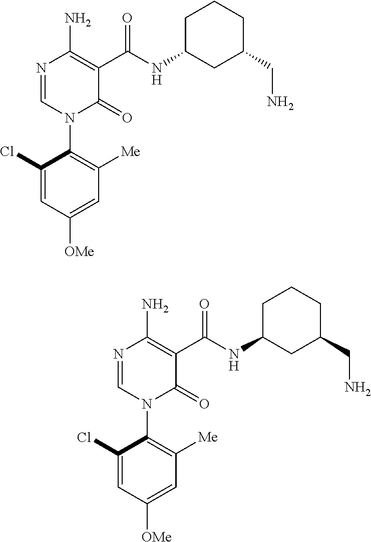

### Step 1. Synthesis of 4-amino-1-((S)-2-chloro-4-methoxy-6-methylphenyl)-N-((1R,3S)-3-(((2,4-dimethoxybenzyl)amino)methyl)cyclohexyl)-6-oxo-1,6-dihydropyrimidine-5-carboxamide or 4-amino-1-((S)-2-chloro-4-methoxy-6-methylphenyl)-N-((1S,3R)-3-(((2,4-dimethoxybenzyl)amino)methyl)cyclohexyl)-6-oxo-1,6-dihydropyrimidine-5-carboxamide

To a solution of (1S,3R)-3-(((2,4-dimethoxybenzyl)amino)methyl)cyclohexan-1-amine or (1R,3S)-3-(((2,4-dimethoxybenzyl)amino)methyl)cyclohexan-1-amine (Intermediate A58, 130.28 mg, 0.468 mmol) and methyl 4-amino-1-((S)-2-chloro-4-methoxy-6-methylphenyl)-6-oxo-1,6-dihydropyrimidine-5-carboxylate (Intermediate B4, 101 mg, 0.312 mmol) in toluene (2 mL) was added AlMe₃ (2M in toluene, 0.47 mL) at 0° C. under N₂ and the mixture stirred at 100° C. for 1 h under N₂. The reaction mixture was quenched by addition saturated aqueous NaOH solution (2 mL) at 0° C. and the reaction mixture evaporated to dryness. The residue was purified by prep-TLC (10/1 EtOAc/MeOH) to give the title compound as a light yellow oil (70 mg, crude). LCMS m/z=571 [M+H]⁺.

### Step 2. Synthesis of tert-butyl(((1S,3R)-3-(4-amino-1-((S)-2-chloro-4-methoxy-6-methylphenyl)-6-oxo-1,6-dihydropyrimidine-5-carboxamido)cyclohexyl)methyl) (2,4-dimethoxybenzyl)carbamate or tert-butyl(((1R,3S)-3-(4-amino-1-((S)-2-chloro-4-methoxy-6-methylphenyl)-6-oxo-1,6-dihydropyrimidine-5-carboxamido)cyclohexyl)methyl) (2,4-dimethoxybenzyl)carbamate

To a solution of 4-amino-1-((S)-2-chloro-4-methoxy-6-methylphenyl)-N-((1R,3S)-3-(((2,4-dimethoxybenzyl)amino)methyl)cyclohexyl)-6-oxo-1,6-dihydropyrimidine-5-carboxamide or 4-amino-1-((S)-2-chloro-4-methoxy-6-methylphenyl)-N-((1S,3R)-3-(((2,4-dimethoxybenzyl)amino)methyl)cyclohexyl)-6-oxo-1,6-dihydropyrimidine-5-carboxamide (Step 1, 60 mg, 0.105 mmol) in MeOH (2 mL) was added di-tert-butyl dicarbonate (68.9 mg, 0.316 mmol) and the mixture stirred at 25° C. for 1 h. The reaction mixture was concentrated under reduced pressure and the residue purified by prep-TLC (50% PE/EtOAc) to give the title compound as a light yellow oil (80 mg, crude). LCMS m/z=671 [M+H]⁺.

### Step 3. Synthesis of 4-amino-N-((1R,3S)-3-(aminomethyl)cyclohexyl)-1-((S)-2-chloro-4-methoxy-6-methylphenyl)-6-oxo-1,6-dihydropyrimidine-5-carboxamide or 4-amino-N-((1S,3R)-3-(aminomethyl)cyclohexyl)-1-((S)-2-chloro-4-methoxy-6-methylphenyl)-6-oxo-1,6-dihydropyrimidine-5-carboxamide

A solution of tert-butyl(((1S,3R)-3-(4-amino-1-((S)-2-chloro-4-methoxy-6-methylphenyl)-6-oxo-1,6-dihydropyrimidine-5-carboxamido)cyclohexyl)methyl) (2,4-dimethoxybenzyl)carbamate or tert-butyl(((1R,3S)-3-(4-amino-1-((S)-2-chloro-4-methoxy-6-methylphenyl)-6-oxo-1,6-dihydropyrimidine-5-carboxamido)cyclohexyl)methyl) (2,4-dimethoxybenzyl)carbamate (Step 2, 70 mg, 0.104 mmol) in TFA (2 mL) was stirred at 80° C. for 1 h. The reaction mixture was concentrated under N₂ and the residue purified by prep-HPLC-1 (1-50% MeCN) to give the title compound as a white solid (15.8 mg, 36%). LCMS m/z=420 [M+H]⁺; ¹H NMR (400 MHz, CDCl₃): 0.87 (q, 2H), 1.10-1.22 (m, 1H), 1.39 (q, 1H), 1.82 (br d, 3H), 1.98 (br d, 1H), 2.18 (s, 4H), 2.71 (br d, 2H), 3.83 (s, 4H), 6.17 (br d, 1H), 6.79 (d, 1H), 6.93 (d, 1H), 7.66 (s, 1H), 8.49 (br s, 1H), 9.59 (d, 1H), 9.99 (br d, 1H).

### Example 207. Synthesis of (R)-2-(4-(4-amino-6-oxo-5-((5-(piperidin-2-yl)pyridin-3-yl)carbamoyl)pyrimidin-1 (6H)-yl)-3,5-dimethylphenyl) acetic acid

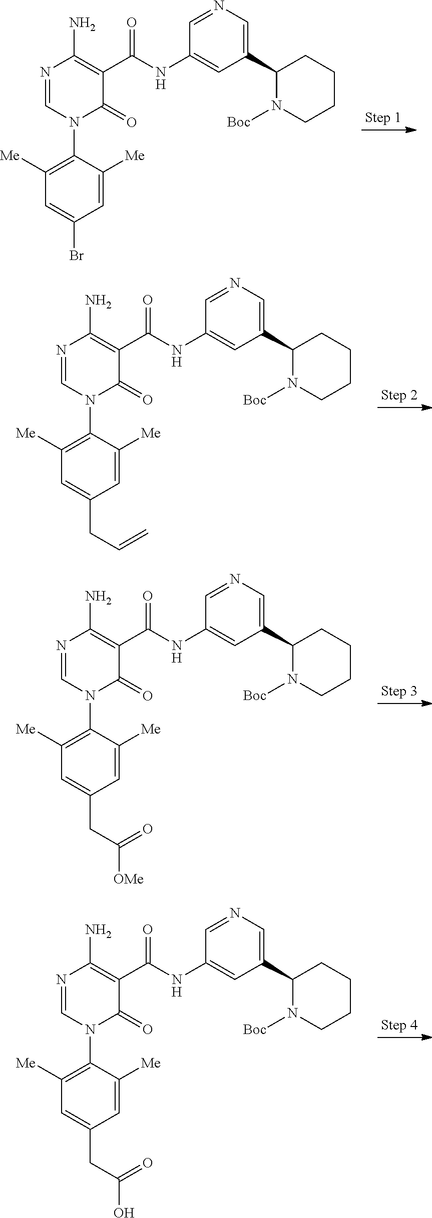 [SMILES: Cc1cc(Br)cc(C)c1-n1cnc(N)c(C(=O)Nc2cncc([C@H]3CCCCN3C(=O)OC(C)(C)C)c2)c1=O ; C=CCc1cc(C)c(-n2cnc(N)c(C(=O)Nc3cncc([C@H]4CCCCN4C(=O)OC(C)(C)C)c3)c2=O)c(C)c1 ; COC(=O)Cc1cc(C)c(-n2cnc(N)c(C(=O)Nc3cncc([C@H]4CCCCN4C(=O)OC(C)(C)C)c3)c2=O)c(C)c1 ; Cc1cc(CC(=O)O)cc(C)c1-n1cnc(N)c(C(=O)Nc2cncc([C@H]3CCCCN3C(=O)OC(C)(C)C)c2)c1=O]

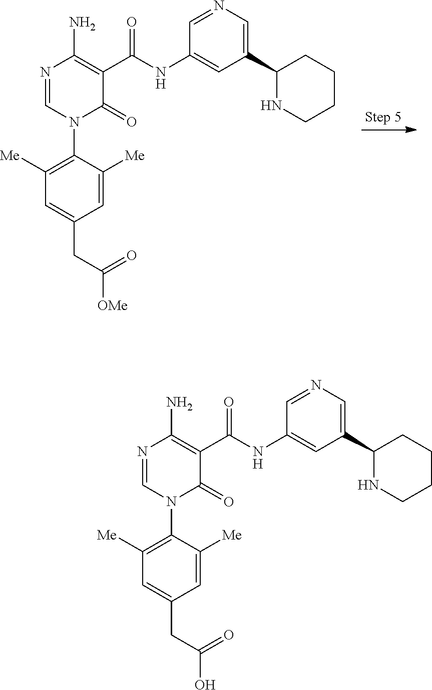 [SMILES: COC(=O)Cc1cc(C)c(-n2cnc(N)c(C(=O)Nc3cncc([C@H]4CCCCN4)c3)c2=O)c(C)c1 ; Cc1cc(CC(=O)O)cc(C)c1-n1cnc(N)c(C(=O)Nc2cncc([C@H]3CCCCN3)c2)c1=O]

### Step 1. Synthesis of tert-butyl(R)-2-(5-(1-(4-allyl-2,6-dimethylphenyl)-4-amino-6-oxo-1,6-dihydropyrimidine-5-carboxamido)pyridin-3-yl) piperidine-1-carboxylate

To a solution of tert-butyl(R)-2-(5-(4-amino-1-(4-bromo-2,6-dimethylphenyl)-6-oxo-1,6-dihydropyrimidine-5-carboxamido)pyridin-3-yl) piperidine-1-carboxylate (Example 205, Step 1, 820 mg, 1.37 mmol) in DMF (5 mL) was added Pd(PPh₃)₄ (159 mg, 0.137 mmol) and allyltributylstannane (500 mg, 1.51 mmol) under N₂ and the mixture stirred at 90° C. for 3 h under N₂. The reaction mixture was quenched by addition saturated aqueous KF solution (30 mL) at 0° C. and extracted with EtOAc (3×20 mL). The combined organic layers were dried (Na₂SO₄) and concentrated under reduced pressure. The residue was purified by MPLC (SiO₂, EtOAc) to give the title compound as a yellow oil (720 mg, 94%). LCMS m/z=559 [M+H]⁺.

### Step 2. Synthesis of tert-butyl(R)-2-(5-(4-amino-1-(4-(2-methoxy-2-oxoethyl)-2,6-dimethylphenyl)-6-oxo-1,6-dihydropyrimidine-5-carboxamido)pyridin-3-yl) piperidine-1-carboxylate

Ozone (15 psi) was bubbled through a solution of tert-butyl(R)-2-(5-(1-(4-allyl-2,6-dimethylphenyl)-4-amino-6-oxo-1,6-dihydropyrimidine-5-carboxamido)pyridin-3-yl) piperidine-1-carboxylate or (Step 1, 280 mg, 0.501 mmol) in DCM (10 mL) and NaOH (2.5 M, 2.65 mL) in MeOH at −78° C. for 0.5 h. The reaction mixture was concentrated under reduced pressure to give the title compound as a white solid (296 mg, crude). LCMS m/z=591 [M+H]⁺.

### Step 3. Synthesis of (R)-2-(4-(4-amino-5-((5-(1-(tert-butoxycarbonyl) piperidin-2-yl)pyridin-3-yl)carbamoyl)-6-oxopyrimidin-1 (6H)-yl)-3,5-dimethylphenyl) acetic acid

To a solution of tert-butyl(R)-2-(5-(4-amino-1-(4-(2-methoxy-2-oxoethyl)-2,6-dimethylphenyl)-6-oxo-1,6-dihydropyrimidine-5-carboxamido)pyridin-3-yl) piperidine-1-carboxylate (Step 2, 296 mg, 0.501 mol) in MeOH (3 mL) and H₂O (1 mL) was added NaOH (60 mg, 1.50 mmol) and the mixture stirred at 25° C. for 1 h. The reaction mixture was filtered and the filtrate was concentrated under reduced pressure and the residue purified by prep-HPLC-13 (25-45% MeCN) to give the title compound as a white solid (68 mg, 23%). LCMS m/z=577 [M+H]⁺.

### Step 4. Synthesis of methyl(R)-2-(4-(4-amino-6-oxo-5-((5-(piperidin-2-yl)pyridin-3-yl)carbamoyl)pyrimidin-1 (6H)-yl)-3,5-dimethylphenyl)acetate

To a solution of (R)-2-(4-(4-amino-5-((5-(1-(tert-butoxycarbonyl) piperidin-2-yl)pyridin-3-yl)carbamoyl)-6-oxopyrimidin-1 (6H)-yl)-3,5-dimethylphenyl) acetic acid (Step 3, 68 mg, 0.118 mol) in HCl/EtOAc (1 mL) was stirred at 25° C. for 1 h. The reaction mixture was concentrated under reduced pressure and the residue was purified by prep-HPLC-1 (5-35% MeCN) to give the title compound as a yellow gum (37 mg, 63%). LCMS m/z=491 [M+H]⁺.

### Step 5. Synthesis of (R)-2-(4-(4-amino-6-oxo-5-((5-(piperidin-2-yl)pyridin-3-yl)carbamoyl)pyrimidin-1 (6H)-yl)-3,5-dimethylphenyl) acetic acid

To a solution of methyl(R)-2-(4-(4-amino-6-oxo-5-((5-(piperidin-2-yl)pyridin-3-yl)carbamoyl)pyrimidin-1 (6H)-yl)-3,5-dimethylphenyl)acetate (Step 4, 32 mg, 0.065 mmol) in MeOH (1 mL) and H₂O (0.3 mL) was added NaOH (7.83 mg, 0.20 mmol) and the mixture stirred at 25° C. for 1 h. The reaction mixture was concentrated under reduced pressure and the pH of the residue adjusted to pH 2-3 with 1M HCl. The residue was purified by prep-HPLC-1 (5-40% MeCN) to give the title compound as a white solid (9.1 mg, 26%). LCMS m/z=477 [M+H]⁺; ¹H NMR (400 MHz, DMSO-d₃): 12.14 (s, 1H), 9.52 (d, 1H), 8.68 (d, 1H), 8.51 (d, 1H), 8.25 (d, 2H), 8.09 (s, 1H), 7.13 (s, 2H), 3.70 (d, 1H), 3.54 (s, 2H), 3.09 (d, 1H), 2.69 (d, 1H), 2.06 (s, 6H), 1.84-1.70 (m, 2H), 1.60 (d, 1H), 1.45 (s, 3H).

### Example 208. Synthesis of (R)-4-amino-N-(5-(1-amino-2-methoxyethyl)pyridin-3-yl)-1-(2,6-dichloro-4-cyclopropoxyphenyl)-6-oxo-1,6-dihydropyrimidine-5-carboxamide or (S)-4-amino-N-(5-(1-amino-2-methoxyethyl)pyridin-3-yl)-1-(2,6-dichloro-4-cyclopropoxyphenyl)-6-oxo-1,6-dihydropyrimidine-5-carboxamide

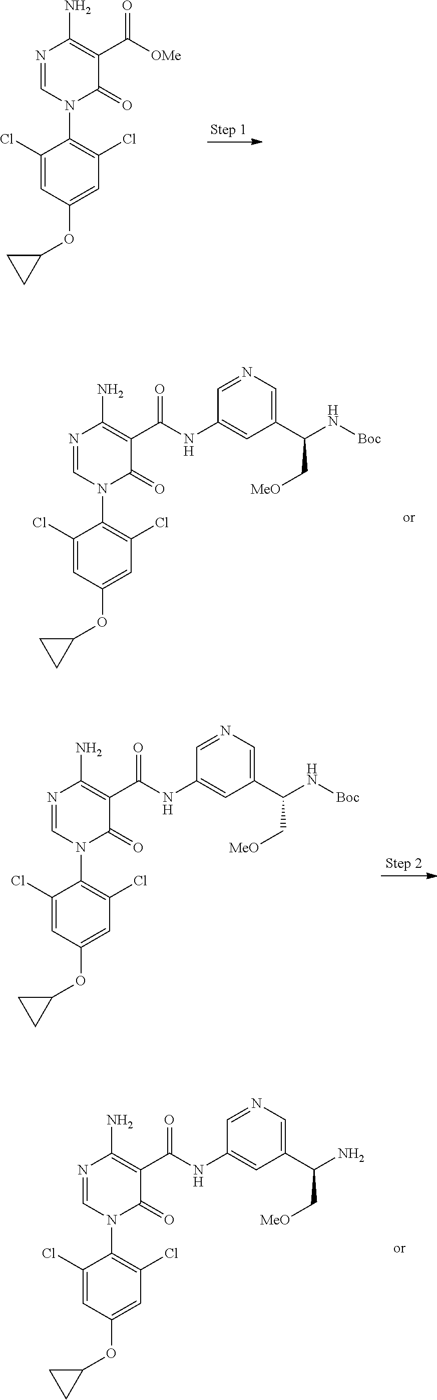 [SMILES: COC(=O)c1c(N)ncn(-c2c(Cl)cc(OC3CC3)cc2Cl)c1=O ; COC[C@H](NC(=O)OC(C)(C)C)c1cncc(NC(=O)c2c(N)ncn(-c3c(Cl)cc(OC4CC4)cc3Cl)c2=O)c1 ; COC[C@@H](NC(=O)OC(C)(C)C)c1cncc(NC(=O)c2c(N)ncn(-c3c(Cl)cc(OC4CC4)cc3Cl)c2=O)c1 ; COC[C@H](N)c1cncc(NC(=O)c2c(N)ncn(-c3c(Cl)cc(OC4CC4)cc3Cl)c2=O)c1]

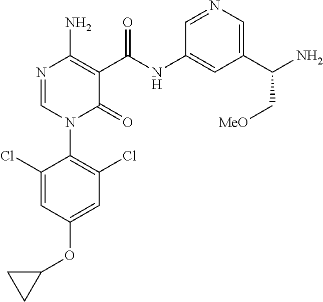 [SMILES: COC[C@@H](N)c1cncc(NC(=O)c2c(N)ncn(-c3c(Cl)cc(OC4CC4)cc3Cl)c2=O)c1]

### Step 1. Synthesis of tert-butyl (R)-(1-(5-(4-amino-1-(2,6-dichloro-4-cyclopropoxyphenyl)-6-oxo-1,6-dihydropyrimidine-5-carboxamido)pyridin-3-yl)-2-methoxyethyl)carbamate or tert-butyl(S)-(1-(5-(4-amino-1-(2,6-dichloro-4-cyclopropoxyphenyl)-6-oxo-1,6-dihydropyrimidine-5-carboxamido)pyridin-3-yl)-2-methoxyethyl)carbamate

To a solution of methyl 4-amino-1-[2,6-dichloro-4-(cyclopropoxy)phenyl]-6-oxo-pyrimidine-5-carboxylate (Intermediate B11, 30 mg, 81 μmol) and tert-butyl (R)-(1-(5-aminopyridin-3-yl)-2-methoxyethyl)carbamate or tert-butyl(S)-(1-(5-aminopyridin-3-yl)-2-methoxyethyl)carbamate (Intermediate A22, 32.5 mg, 0.122 mmol) in toluene (1.5 mL) and THF (0.5 mL) was added AlMe₃ (2M in toluene, 0.122 mL) at 0° C. and the mixture was stirred at 60° C. for 1 h under N₂. The reaction mixture was quenched by the addition of TFA (0.2 mL) at 0° C. and the mixture concentrated under reduced pressure to the title compound as a brown oil (40 mg, 81%). LCMS m/z=605 [M+H].

### Step 2. Synthesis of (R)-4-amino-N-(5-(1-amino-2-methoxyethyl)pyridin-3-yl)-1-(2,6-dichloro-4-cyclopropoxyphenyl)-6-oxo-1,6-dihydropyrimidine-5-carboxamide or (S)-4-amino-N-(5-(1-amino-2-methoxyethyl)pyridin-3-yl)-1-(2,6-dichloro-4-cyclopropoxyphenyl)-6-oxo-1,6-dihydropyrimidine-5-carboxamide

To a solution of tert-butyl (R)-(1-(5-(4-amino-1-(2,6-dichloro-4-cyclopropoxyphenyl)-6-oxo-1,6-dihydropyrimidine-5-carboxamido)pyridin-3-yl)-2-methoxyethyl)carbamate or tert-butyl(S) (1-(5-(4-amino-1-(2,6-dichloro-4-cyclopropoxyphenyl)-6-oxo-1,6-dihydropyrimidine-5-carboxamido)pyridin-3-yl)-2-methoxyethyl)carbamate (Step 1, 40 mg, 66 μmol) in DCM (1 mL) was added TFA (1 mL) and the mixture stirred at 25° C. for 1 h. The reaction mixture was concentrated under reduced pressure and the residue purified by prep-HPLC-1 (3-35% MeCN) to give the title compound as a pale yellow solid (9.9 mg, 27%). LCMS m/z=505 [M+H]⁺; ¹H NMR (400 MHz, CDCl₃): 11.78 (s, 1H), 10.12-9.95 (m, 1H), 8.70 (s, 1H), 8.33 (s, 1H), 8.20 (s, 1H), 7.73 (s, 1H), 7.22 (s, 2H), 6.08 (s, 1H), 4.28-4.17 (m, 1H), 3.79 (s, 1H), 3.52 (dd, 1H), 3.39 (s, 3H), 0.86 (d, 5H).

### Example 209. Synthesis of (S)-4-amino-1-(4-(azetidin-1-yl)-2,6-dichlorophenyl)-6-oxo-N-(5-(piperidin-2-yl)pyridin-3-yl)-1,6-dihydropyrimidine-5-carboxamide

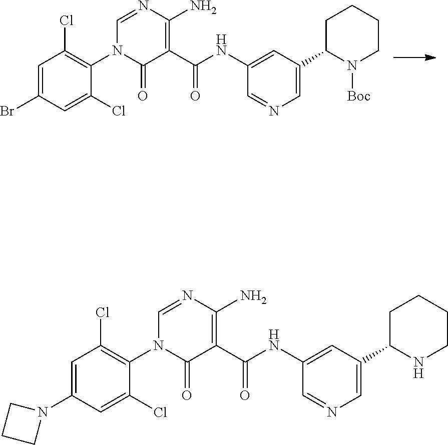 [SMILES: CC(C)(C)OC(=O)N1CCCC[C@H]1c1cncc(NC(=O)c2c(N)ncn(-c3c(Cl)cc(Br)cc3Cl)c2=O)c1 ; Nc1ncn(-c2c(Cl)cc(N3CCC3)cc2Cl)c(=O)c1C(=O)Nc1cncc([C@@H]2CCCCN2)c1]

To a solution of tert-butyl(S)-2-(5-(4-amino-1-(4-bromo-2,6-dichlorophenyl)-6-oxo-1,6-dihydropyrimidine-5-carboxamido)pyridin-3-yl) piperidine-1-carboxylate (Example 196, Step 1; 40 mg, 0.063 mmol) in dioxane (3 mL) was added Pd₂ (dba)₃ (5.74 mg, 0.0063 mmol), Xantphos (3.63 mg, 0.0063 mmol) and NaO′Bu (12 mg, 0.125 mmol) and the mixture stirred at 100° C. for 1 h under N₂. The reaction mixture was filtered and the filtrate was concentrated under reduced pressure. The residue was purified by prep-TLC (100% EtOAc) to give a white solid which was dissolved in TFA (1 mL) and DCM (1 mL). The mixture was stirred at 25° C. for 1 h. The reaction mixture was concentrated under N₂ and the residue purified by prep-HPLC-1 (15-45% MeCN) to afford the title compound as a pale yellow solid (6.8 mg, 20%). LCMS m/z=514 [M+H]⁺; ¹H NMR (400 MHz, CDCl₃): 11.89 (s, 1H), 9.98 (d, 1H), 8.62 (d, 1H), 8.33 (s, 2H), 7.74 (s, 1H), 6.43 (s, 2H), 5.99 (d, 1H), 3.98 (t, 4H), 3.76 (dd, 1H), 3.18 (d, 2H), 2.82-2.75 (m, 1H), 2.49-2.41 (m, 2H), 1.94-1.85 (m, 2H), 1.73-1.68 (m, 3H), 1.53-1.48 (m, 1H).

### Example 210. Synthesis of (R)—N-(5-(1,4-oxazepan-3-yl)pyridin-3-yl)-4-amino-1-(2,6-dichlorophenyl)-6-oxo-1,6-dihydropyrimidine-5-carboxamide or (S)—N-(5-(1,4-oxazepan-3-yl)pyridin-3-yl)-4-amino-1-(2,6-dichlorophenyl)-6-oxo-1,6-dihydropyrimidine-5-carboxamide

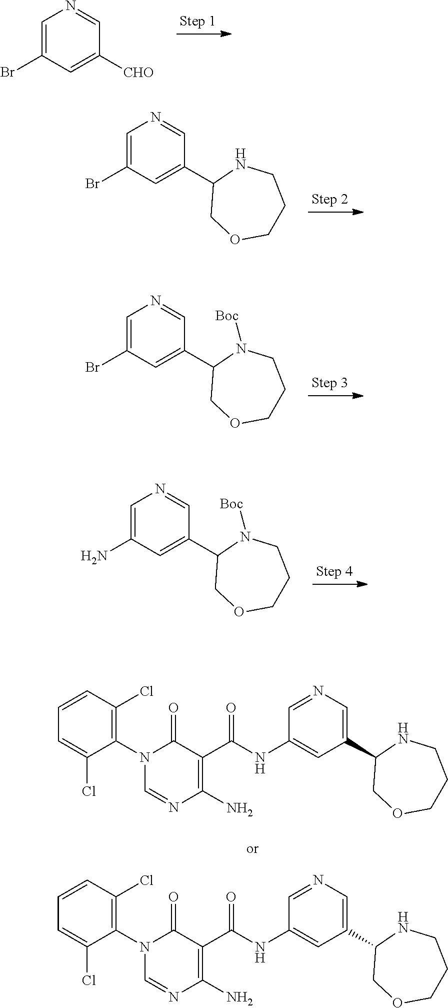 [SMILES: O=Cc1cncc(Br)c1 ; Brc1cncc(C2COCCCN2)c1 ; CC(C)(C)OC(=O)N1CCCOCC1c1cncc(Br)c1 ; CC(C)(C)OC(=O)N1CCCOCC1c1cncc(N)c1 ; Nc1ncn(-c2c(Cl)cccc2Cl)c(=O)c1C(=O)Nc1cncc([C@@H]2COCCCN2)c1 ; Nc1ncn(-c2c(Cl)cccc2Cl)c(=O)c1C(=O)Nc1cncc([C@H]2COCCCN2)c1]

### Step 1. Synthesis of 3-(5-bromopyridin-3-yl)-1,4-oxazepane

To a solution of the 3-((tributylstannyl) methoxy)propan-1-amine (1135 mg, 3.00 mmol) in DCM (15 mL) was added 5-bromonicotinaldehyde (558 mg, 3.0 mmol) and 4 Å molecular sieves (350 mg) under an inert atmosphere at ambient temperature. The reaction mixture was stirred for 2 h and filtered through a pad of Celite rinsing with DCM (50 mL). The filtrate was concentrated under reduced pressure to afford 1-(5-bromopyridin-3-yl)-N-(3-((tributylstannyl) methoxy) propyl) methanimine. Separately, anhydrous Copper (II) trifluoromethanesulfonate (1085 mg, 3.0 mmol) was added to a solution of 2,6-lutidine (0.35 mL) in 1,1,1,3,3,3-hexafluoropropan-2-ol (12 mL) in a dry Schlenk flask and stirred at RT for 1 h, during which a homogeneous suspension formed. A solution of 1-(5-bromopyridin-3-yl)-N-(3-((tributylstannyl) methoxy) propyl) methanimine in DCM (48 mL) was added in one portion and the resulting mixture was allowed to stir at RT for 36 h. The reaction was quenched with a mixture of NaHCO₃ (24 mL) and 10% aqueous NH₄OH (12 mL) and stirred vigorously for 15 min. The layers were separated and the aqueous layer extracted with DCM (3×10 mL). The combined organics were washed with water (3×10 mL), brine (5 mL), dried (Na₂SO₄) and evaporated to dryness in vacuo. Flash chromatography (ISCO, 24 g silica, 0-15% MeOH/DCM) provided the title compound as an orange oil (337 mg, 44%). LCMS m/z=257 [M+H]⁺.

### Step 2. Synthesis of tert-butyl 3-(5-bromopyridin-3-yl)-1,4-oxazepane-4-carboxylate

To a solution of 3-(5-bromopyridin-3-yl)-1,4-oxazepane (Step 1, 337 mg, 1.311 mmol) in DCM (2.6 mL) was added (Boc)₂₀ (335 μl, 1.44 mmol), DMAP (8.0 mg, 0.066 mmol) and TEA (274 μl, 1.96 mmol) and the reaction stirred at RT for a 16 h. The mixture was poured into water (10 mL) and the aqueous layer extracted with DCM (3×10 mL). The combined organics were washed with NH₄OH (10 mL) and brine (10 mL), dried (Na₂SO₄) and evaporated to dryness. The residue was purified by flash chromatography (ISCO, 24 g silica, 10-60% EtOAc/Hex) to afford the title compound as an off-white solid (249 mg, 53%). LCMS m/z=395 [M+K]⁺.

### Step 3. Synthesis of tert-butyl 3-(5-aminopyridin-3-yl)-1,4-oxazepane-4-carboxylate

To a suspension of tert-butyl 3-(5-bromopyridin-3-yl)-1,4-oxazepane-4-carboxylate (Step 2, 248 mg, 0.694 mmol), Cs₂CO₃ (339 mg, 1.041 mmol), Pd₂ (dba)₃ (31.8 mg, 0.035 mmol), and BINAP (43.2 mg, 0.069 mmol) in anhydrous toluene (2.75 mL) was added benzophenone imine (144 μL, 0.83 mmol) and the mixture sparged with N₂ for 10 min and then heated to 80° C. for 15 h. The reaction was filtered through Celite, washing with EtOAc and the filtrate evaporated to dryness. The residue was purified by flash chromatography (ISCO, 24 g silica, 0-60% EtOAc/Hex) to afford tert-butyl 3-(5-((diphenylmethylene)amino)pyridin-3-yl)-1,4-oxazepane-4-carboxylate intermediate as viscous yellow oil (322 mg, 91% yield) which was dissolved in MeOH (10.5 mL) and cooled to 0° C. Hydroxylamine hydrochloride (132 mg, 1.90 mmol) and NaOAc (260 mg, 3.17 mmol) were added and the reaction mixture stirred at RT for 2 h. The reaction mixture was poured into aqueous NH₄Cl and extracted with EtOAc (3×50 mL). The combined organics were washed with brine, dried (Na₂SO₄) and concentrated by rotary evaporation. The residue was purified by flash chromatography (ISCO, 24 g silica, 0-15% MeOH (w/10% NH₄OH)/DCM) to afford the title compound as a white solid (162 mg, 87%). LCMS m/z=294 [M+H]⁺;

### Step 4. Synthesis of (R)—N-(5-(1,4-oxazepan-3-yl)pyridin-3-yl)-4-amino-1-(2,6-dichlorophenyl)-6-oxo-1,6-dihydropyrimidine-5-carboxamide or (S)—N-(5-(1,4-oxazepan-3-yl)pyridin-3-yl)-4-amino-1-(2,6-dichlorophenyl)-6-oxo-1,6-dihydropyrimidine-5-carboxamide

Methyl 4-amino-1-(2,6-dichlorophenyl)-6-oxo-1,6-dihydropyrimidine-5-carboxylate (Intermediate B9, 55 mg, 0.175 mmol) and tert-butyl 3-(5-aminopyridin-3-yl)-1,4-oxazepane-4-carboxylate MEN-005-082 (Step 3, 61.6 mg, 0.210 mmol) were added to a dry flask and evacuated and backfilled (×3) with N₂. Anhydrous toluene (3.5 mL) was added and the solution cooled to 0° C. and AlMe₃ solution (2M in toluene, 263 L, 0.525 mmol) added dropwise. The reaction was stirred at ambient temperature for 10 min and at 100° C. for 1.5 h. The reaction mixture was quenched with MeOH (2 mL), TFA (0.2 mL), poured into water (15 mL) and extracted with DCM (4×10 mL). The combined organics were washed with sat aq sodium bicarbonate, water, brine, dried (Na₂SO₄) and evaporated to dryness. The residue was dissolved in DCM (1.7 mL) and TFA (43.8 μL, 0.191 mmol) added dropwise. The mixture was stirred at RT for 2 h and heated to 40° C. for 16 h. The reaction mixture was evaporated to dryness in vacuo and the residue purified by flash chromatography (ISCO, 12 g silica, 0-15% MeOH (w/10% NH₄OH)/DCM) followed by chiral SFC (Regis Whelk O-1 (S,S) 21×250 mm, 45% EtOH+0.25% diethylamine in CO₂) provided

Peak 1, Example 210. (R)—N-(3-(1,4-oxazepan-3-yl)phenyl)-4-amino-1-(2,6-dichlorophenyl)-6-oxo-1,6-dihydropyrimidine-5-carboxamide or (S)—N-(5-(1,4-oxazepan-5-yl)pyridin-3-yl)-4-amino-1-(2,6-dichlorophenyl)-6-oxo-1,6-dihydropyrimidine-5-carboxamide (white solid, 21 mg, 25%); LCMS m/z=476 [M+H]⁺; ¹H NMR (500 MHz, DMSO-d₆): 11.84 (s, 1H), 9.71 (s, 1H), 8.91-8.70 (m, 2H), 8.52 (s, 1H), 8.32 (d, 1H), 8.05 (d, 1H), 7.83 (d, 2H), 7.71 (t, 1H), 4.03-3.81 (m, 3H), 3.76 (dt, 1H), 3.47-3.42 (m, 1H), 3.11 (d, 1H), 2.88 (dt, 1H), 1.91 (t, 2H).

### Example 211. Synthesis of (R)—N-(3-(1,4-oxazepan-3-yl)phenyl)-4-amino-1-(2,6-dichlorophenyl)-6-oxo-1,6-dihydropyrimidine-5-carboxamide or (S)—N-(3-(1,4-oxazepan-3-yl)phenyl)-4-amino-1-(2,6-dichlorophenyl)-6-oxo-1,6-dihydropyrimidine-5-carboxamide

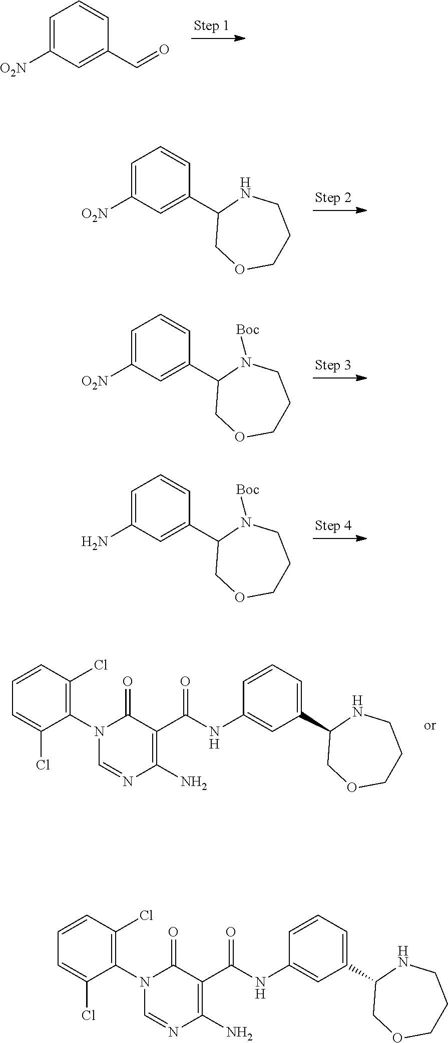 [SMILES: O=Cc1cccc([N+](=O)[O-])c1 ; O=[N+]([O-])c1cccc(C2COCCCN2)c1 ; CC(C)(C)OC(=O)N1CCCOCC1c1cccc([N+](=O)[O-])c1 ; CC(C)(C)OC(=O)N1CCCOCC1c1cccc(N)c1 ; Nc1ncn(-c2c(Cl)cccc2Cl)c(=O)c1C(=O)Nc1cccc([C@@H]2COCCCN2)c1 ; Nc1ncn(-c2c(Cl)cccc2Cl)c(=O)c1C(=O)Nc1cccc([C@H]2COCCCN2)c1]

### Step 1. Synthesis of 3-(3-nitrophenyl)-1,4-oxazepane

The title compound was prepared as a white solid (152 mg, 45%) using an analogous method to that described for Example 210, Step 1. LCMS m/z=223 [M+H]⁺.

### Step 2. Synthesis of tert-butyl 3-(3-nitrophenyl)-1,4-oxazepane-4-carboxylate

The title compound was prepared as a pale yellow oil (125 mg, 57%) using an analogous method to that described for Example 210, Step 2. LCMS m/z=345 [M+Na]⁺.

### Step 3. Synthesis of tert-butyl 3-(3-aminophenyl)-1,4-oxazepane-4-carboxylate

To a mixture of tert-butyl 3-(3-nitrophenyl)-1,4-oxazepane-4-carboxylate (Step 2, 125 mg, 0.400 mmol) in THF (3 mL) and water (1.75 mL) was added Fe (112 mg, 2.00 mmol) and ammonium chloride (107 mg, 2.00 mmol) and the mixture heated to 60° C. and stirred overnight. The reaction was cooled and filtered through celite, washed with EtOAc. The filtrate was washed with aqueous NaHCO₃ (10 mL) and brine (10 mL), dried (Na₂SO₄) and evaporated to dryness in vacuo to afford the title compound as a yellow solid (103 mg, 88%). LCMS m/z=315 [M+Na]⁺.

### Step 4. Synthesis of (R)—N-(3-(1,4-oxazepan-3-yl)phenyl)-4-amino-1-(2,6-dichlorophenyl)-6-oxo-1,6-dihydropyrimidine-5-carboxamide or (S)—N-(3-(1,4-oxazepan-3-yl)phenyl)-4-amino-1-(2,6-dichlorophenyl)-6-oxo-1,6-dihydropyrimidine-5-carboxamide

The title compound was prepared as a white solid (11.1 mg, 12%) using an analogous method to that described for Example 210, Step 4. Chiral SFC (Regis Whelk O-1 (S,S) 21×250 mm, 35% EtOH+0.25% diethylamine in CO₂) provided:

Peak 1, Example 211. (R)—N-(3-(1,4-oxazepan-3-yl)phenyl)-4-amino-1-(2,6-dichlorophenyl)-6-oxo-1,6-dihydropyrimidine-5-carboxamide or (S)—N-(3-(1,4-oxazepan-3-yl)phenyl)-4-amino-1-(2,6-dichlorophenyl)-6-oxo-1,6-dihydropyrimidine-5-carboxamide (white solid, 11.1 mg, 12%); LCMS m/z=474 [M+H]⁺; ¹H NMR (500 MHz, DMSO-d₆): 11.74 (s, 1H), 9.74 (s, 1H), 8.67 (s, 1H), 8.44 (d, 1H), 7.79 (d, 2H), 7.69-7.59 (m, 2H), 7.49 (d, 1H), 7.26 (t, 1H), 7.05 (d, 1H), 3.90-3.76 (m, 3H), 3.70 (dt, 1H), 3.06 (s, 1H), 2.81 (dt, 1H), 1.86 (q, 2H), 1.06 (d, 2H).

### Example 212. Synthesis of (S)-4-amino-1-(2,6-dichloro-4-methoxyphenyl)-N-(5-(morpholin-3-yl)pyridin-3-yl)-6-oxo-1,6-dihydropyrimidine-5-carboxamide or (R)-4-amino-1-(2,6-dichloro-4-methoxyphenyl)-N-(5-(morpholin-3-yl)pyridin-3-yl)-6-oxo-1,6-dihydropyrimidine-5-carboxamide

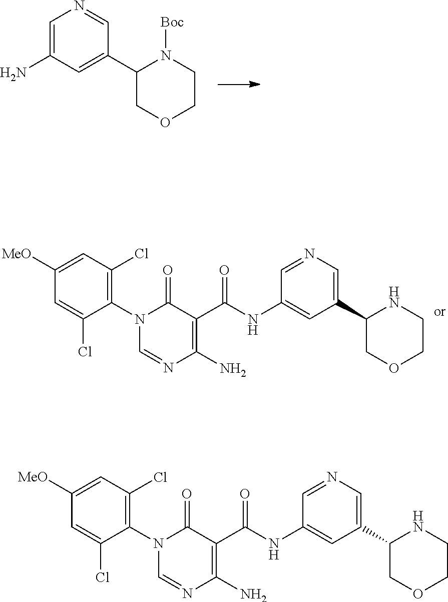 [SMILES: CC(C)(C)OC(=O)N1CCOCC1c1cncc(N)c1 ; COc1cc(Cl)c(-n2cnc(N)c(C(=O)Nc3cncc([C@@H]4COCCN4)c3)c2=O)c(Cl)c1 ; COc1cc(Cl)c(-n2cnc(N)c(C(=O)Nc3cncc([C@H]4COCCN4)c3)c2=O)c(Cl)c1]

Methyl 4-amino-1-(2,6-dichloro-4-methoxyphenyl)-6-oxo-1,6-dihydropyrimidine-5-carboxylate (Intermediate B10, 60 mg, 0.175 mmol) and tert-butyl 3-(5-aminopyridin-3-yl) morpholine-4-carboxylate (Intermediate A68, 58.4 mg, 0.210 mmol) were added to a dry flask and evacuated and backfilled with N₂ (3×). Anhydrous toluene (3.5 mL) was added and the solution cooled to 0° C. and AlMe₃ (2M in toluene; 262 μL, 0.525 mmol) added dropwise. The reaction was removed from the ice bath and stirred at rt for 10 min and then at 100° C. for 1.5 h. The reaction was cooled to rt and quenched with MeOH (2 mL) and TFA (0.2 mL) poured into water (15 mL) and extracted with DCM (4×10 mL). The combined organics were washed with sat. aq. NaHCO₃, water, brine, dried (Na₂SO₄) and evaporated to dryness. The residue was dissolved in EtOAc (2.5 mL) and HCl (4M in dioxane, 218 μL, 0.872 mmol) added dropwise. The mixture was stirred at rt for 18 h and evaporated to dryness in vacuo. The residue was purified by flash chromatography (ISCO, 12 g silica, 0-10% MeOH (w/10% NH₄OH) in DCM) followed by chiral SFC (ChiralPak AD-H 21×250 mm, 40% MeOH+0.25% diethylamine in CO₂) afforded:

Peak 1, Example 212. (S)-4-amino-1-(2,6-dichloro-4-methoxyphenyl)-N-(5-(morpholin-3-yl)pyridin-3-yl)-6-oxo-1,6-dihydropyrimidine-5-carboxamide or (R)-4-amino-1-(2,6-dichloro-4-methoxyphenyl)-N-(5-(morpholin-3-yl)pyridin-3-yl)-6-oxo-1,6-dihydropyrimidine-5-carboxamide (white solid, 21.9 mg, 26%); LCMS m/z=492 [M+H]; ¹H NMR (500 MHz, DMSO-d₆): 11.79 (s, 1H), 10.03 (s, 1H), 8.74 (d, 1H), 8.33 (d, 1H), 8.19 (s, 1H), 7.73 (s, 1H), 7.06 (s, 2H), 5.98 (s, 1H), 3.97 (dd, 1H), 3.91-3.79 (m, 5H), 3.66 (t, 1H), 3.41 (t, 1H), 3.20-3.06 (m, 1H), 3.01 (d, 1H).

### Example 213. Synthesis of (S)-4-amino-N-(5-(azetidin-2-yl)pyridin-3-yl)-1-(2,6-dichloro-4-methoxyphenyl)-6-oxo-1,6-dihydropyrimidine-5-carboxamide or (R)-4-amino-N-(5-(azetidin-2-yl)pyridin-3-yl)-1-(2,6-dichloro-4-methoxyphenyl)-6-oxo-1,6-dihydropyrimidine-5-carboxamide

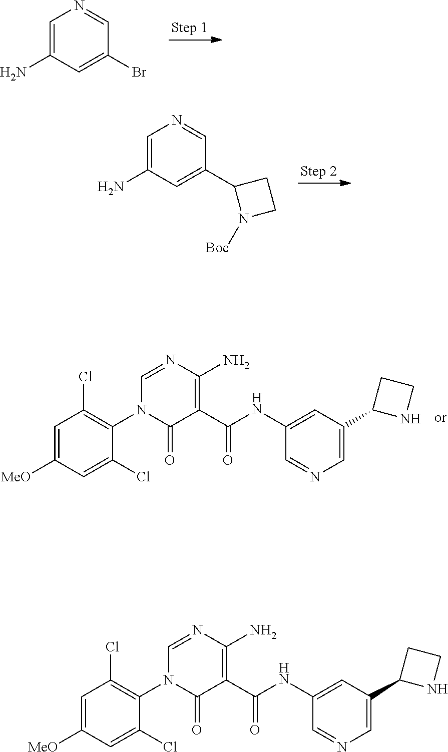 [SMILES: Nc1cncc(Br)c1 ; CC(C)(C)OC(=O)N1CCC1c1cncc(N)c1 ; COc1cc(Cl)c(-n2cnc(N)c(C(=O)Nc3cncc([C@@H]4CCN4)c3)c2=O)c(Cl)c1 ; COc1cc(Cl)c(-n2cnc(N)c(C(=O)Nc3cncc([C@H]4CCN4)c3)c2=O)c(Cl)c1]

### Step 1. Synthesis of tert-butyl 2-(5-aminopyridin-3-yl) azetidine-1-carboxylate

A suspension of 5-bromopyridin-3-amine (77.1 mg, 0.45 mmol), 1-(tert-butoxycarbonyl) azetidine-2-carboxylic acid (214 mg, 0.90 mmol), Cs₂CO₃ (448 mg, 1.35 mmol), 4,4′-di-tert-butyl-2,2′-dipyridyl (18.4 mg, 0.068 mmol), NiCl₂ DME adduct (10.1 mg, 0.045 mmol) and [Ir[dF(CF₃)ppy]2 (dtbbpy)]PF₆ (5.0 mg, 0.0045 mmol) in anhydrous DMF (15 mL) was purged with N₂ for 20 min and then stirred at rt for 72 h in a Penn PhD M2 photoreactor using 450 nM blue LED light. The reaction was filtered through Celite washed with EtOAc (50 mL). The filtrate was washed with water (3×) and brine and evaporated to dryness. The residue was purified by flash chromatography (ISCO, 12 g silica, 0-10% MeOH/DCM) provided the title compound as a yellow-orange oil (44.3 mg, 39%) which was used without further purification in Step 2.

### Step 2. Synthesis of (S)-4-amino-N-(5-(azetidin-2-yl)pyridin-3-yl)-1-(2,6-dichloro-4-methoxyphenyl)-6-oxo-1,6-dihydropyrimidine-5-carboxamide or (R)-4-amino-N-(5-(azetidin-2-yl)pyridin-3-yl)-1-(2,6-dichloro-4-methoxyphenyl)-6-oxo-1,6-dihydropyrimidine-5-carboxamide

A dry vial containing methyl 4-amino-1-(2,6-dichloro-4-methoxyphenyl)-6-oxo-1,6-dihydropyrimidine-5-carboxylate (Intermediate B10, 41.4 mg, 0.120 mmol) and tert-butyl 2-(5-aminopyridin-3-yl) azetidine-1-carboxylate (Step 1, 43.4 mg, 0.168 mmol) was evacuated and backfilled with N₂ (3×). Anhydrous toluene (1.5 mL) was added and the mixture cooled on ice. To this solution was added dropwise AlMe₃ solution (2.0 M in toluene, 0.180 mL). The reaction was stirred on ice for 5 min, rt for 5 minutes and then at 100° C. for 1 h. The reaction was cooled, diluted with DCM (5 mL) and quenched with MeOH (1 mL) and TFA (0.2 mL). The mixture was poured into water and extracted with DCM (4×10 mL). The combined organics were washed with sat aq NaHCO₃, sat aq Rochelle salt, brine, dried (MgSO₄) and concentrated by rotary evaporation. The residue was purified by prep-HPLC-1 (1-50% MeCN) followed by chiral-SFC (REGIS (S,S) WHELK-01 250×25 mm, 10 μm; 50% MeOH (+0.1% NH₄OH) in CO₂)) to afford:

Peak 1, Example 213; (S)-4-amino-N-(5-(azetidin-2-yl)pyridin-3-yl)-1-(2,6-dichloro-4-methoxyphenyl)-6-oxo-1,6-dihydropyrimidine-5-carboxamide or (R)-4-amino-N-(5-(azetidin-2-yl)pyridin-3-yl)-1-(2,6-dichloro-4-methoxyphenyl)-6-oxo-1,6-dihydropyrimidine-5-carboxamide as a white solid (4.7 mg, 27%). LCMS m/z=461 [M+H]⁺; ¹H NMR (400 MHz, CDCl₃): 11.82 (s, 1H), 10.02 (d, 1H), 8.66 (s, 1H), 8.29 (s, 2H), 7.73 (s, 1H), 7.07 (s, 2H), 6.07 (d, 1H), 5.09 (t, 1H), 3.88 (s, 3H), 3.87-3.81 (m, 1H), 3.50 (td, 1H), 2.69-2.58 (m, 1H), 2.47 (dd, 1H).

### Example 214. Synthesis of 4-amino-1-(2,6-dichloro-4-methoxyphenyl)-N-(5-((2S,4R)-4-methylazetidin-2-yl)pyridin-3-yl)-6-oxo-1,6-dihydropyrimidine-5-carboxamide formate or 4-amino-1-(2,6-dichloro-4-methoxyphenyl)-N-(5-((2R,4S)-4-methylazetidin-2-yl)pyridin-3-yl)-6-oxo-1,6-dihydropyrimidine-5-carboxamide formate

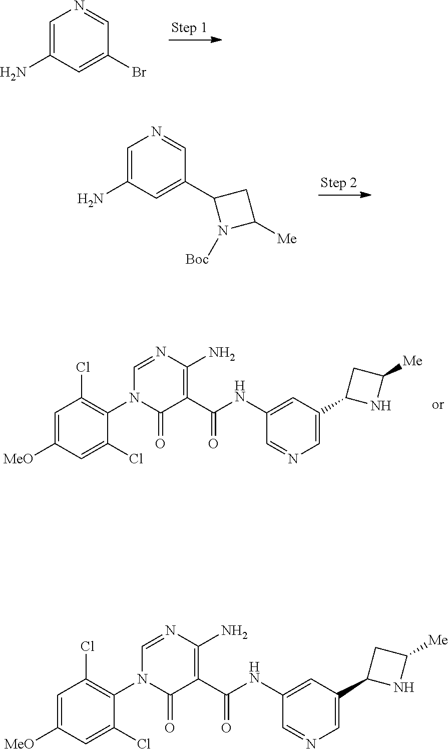 [SMILES: Nc1cncc(Br)c1 ; CC1CC(c2cncc(N)c2)N1C(=O)OC(C)(C)C ; COc1cc(Cl)c(-n2cnc(N)c(C(=O)Nc3cncc([C@@H]4C[C@@H](C)N4)c3)c2=O)c(Cl)c1 ; COc1cc(Cl)c(-n2cnc(N)c(C(=O)Nc3cncc([C@H]4C[C@H](C)N4)c3)c2=O)c(Cl)c1]

### Step 1. Synthesis of tert-butyl 2-(5-aminopyridin-3-yl)-4-methylazetidine-1-carboxylate trifluoroacetate

The title compound was prepared from 1-(tert-butoxycarbonyl)-4-methylazetidine-2-carboxylic acid as a mixture of diastereomers as a yellow oil (91 mg, 60%) using an analogous method to that described for Example 213, Step 1. Prep-HPLC-12 (10-40% MeCN). LCMS m/z=264 [M+H]⁺.

### Step 2. Synthesis of 4-amino-1-(2,6-dichloro-4-methoxyphenyl)-N-(5-((2S,4R)-4-methylazetidin-2-yl)pyridin-3-yl)-6-oxo-1,6-dihydropyrimidine-5-carboxamide formate or 4-amino-1-(2,6-dichloro-4-methoxyphenyl)-N-(5-((2R,4S)-4-methylazetidin-2-yl)pyridin-3-yl)-6-oxo-1,6-dihydropyrimidine-5-carboxamide formate

A suspension of tert-butyl 2-(5-aminopyridin-3-yl)-4-methylazetidine-1-carboxylate trifluoroacetate (88 mg, 0.240 mmol) and K₂CO₃ (202 mg, excess) in MeCN (3 mL) was stirred at rt for 20 min. The mixture was filtered, washed with MeCN and the filtrate evaporated to dryness. A mixture of the residue and methyl 4-amino-1-(2,6-dichloro-4-methoxyphenyl)-6-oxo-1,6-dihydropyrimidine-5-carboxylate (Intermediate B10, 60 mg, 0.171 mmol) was evacuated and backfilled with N₂ (3×). Anhydrous toluene (2.1 mL) was added and the mixture cooled on ice. To this was added dropwise AlMes solution (2.0 M in toluene, 0.514 mmol) and the reaction stirred on ice for 5 min, rt for 5 min and at 100° C. for 1 h. The reaction was cooled, diluted with DCM (5 mL) and quenched with MeOH (1.0 mL) and TFA (0.1 mL). The mixture was poured into water and extracted with DCM (4×15 mL). The combined organics were washed with sat aq NaHCO₃, saturated aq Rochelle salt, brine, dried (MgSO₄) and evaporated to dryness. The residue was dissolved in DCM (3 mL) and TFA (0.6 mL) and stirred at rt for 2 h and evaporated to dryness in vacuo. The residue was purified by reverse-phase HPLC-12 (10-50% MeCN) to afford 4-amino-1-(2,6-dichloro-4-methoxyphenyl)-N-(5-(4-methylazetidin-2-yl)pyridin-3-yl)-6-oxo-1,6-dihydropyrimidine-5-carboxamide trifluoroacetate as a light yellow solid as a 1:1 mixture of diastereomers (74.1 mg TFA salt). The TFA salt was dissolved in MeCN (4 mL) and sat aq NaHCO₃ (0.5 mL) and the mixture stirred at rt for 40 min. The solids were removed by filtration and the filtrate evaporated to dryness. The residue was separated by chiral SFC to afford the title compound as a pale-yellow solid (4 mg, 43%). LCMS m/z=475 [M+H]⁺; ¹H NMR (400 MHz, DMSO-d₆): 11.89 (s, 1H), 9.73-9.63 (m, 1H), 8.82-8.73 (m, 1H), 8.68 (d, 1H), 8.46 (s, 1H), 8.29 (s, 1H), 8.19 (s, 1H), 7.43 (s, 2H), 4.94 (t, 1H), 3.94 (s, 3H), 3.84-3.72 (m, 1H), 2.32-2.26 (m, 2H), 1.44-1.37 (m, 3H).

### Example 215. Synthesis of (R)—N-(5-(1,4-oxazepan-5-yl)pyridin-3-yl)-4-amino-1-(2,6-dichlorophenyl)-6-oxo-1,6-dihydropyrimidine-5-carboxamide or (S)—N-(5-(1,4-oxazepan-5-yl)pyridin-3-yl)-4-amino-1-(2,6-dichlorophenyl)-6-oxo-1,6-dihydropyrimidine-5-carboxamide

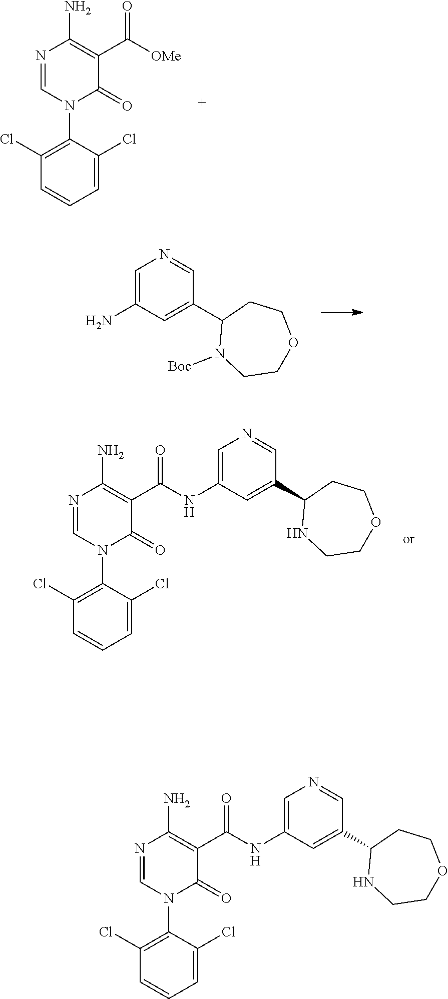 [SMILES: COC(=O)c1c(N)ncn(-c2c(Cl)cccc2Cl)c1=O ; CC(C)(C)OC(=O)N1CCOCCC1c1cncc(N)c1 ; Nc1ncn(-c2c(Cl)cccc2Cl)c(=O)c1C(=O)Nc1cncc([C@H]2CCOCCN2)c1 ; Nc1ncn(-c2c(Cl)cccc2Cl)c(=O)c1C(=O)Nc1cncc([C@@H]2CCOCCN2)c1]

Methyl 4-amino-1-(2,6-dichlorophenyl)-6-oxo-1,6-dihydropyrimidine-5-carboxylate (Intermediate B9, 55 mg, 0.175 mmol) and tert-butyl 5-(5-aminopyridin-3-yl)-1,4-oxazepane-4-carboxylate (Intermediate A28, 61.6 mg, 0.21 mmol) were added to a dry flask and evacuated and backfilled with N₂ (3×). Anhydrous toluene (3.5 mL) was added and the solution was cooled to 0° C. before AlMes solution (2M in toluene, 263 μL, 0.525 mmol) was added dropwise. The reaction was removed from the ice bath and stirred at ambient temperature for 10 min and at 100° C. for 1.5 h. The reaction was cooled to rt and quenched with MeOH (2 mL) and TFA (0.2 mL) poured into H₂O (15 mL) and extracted with DCM (4×10 mL). The combined organics were washed with sat aq NaHCO₃, water, brine, dried (Na₂SO₄) and evaporated to dryness. The residue was dissolved in DCM (1.7 mL) and TFA (13.49 μl, 0.175 mmol) added dropwise. The mixture was stirred at rt for 18 h and evaporated to dryness in vacuo. The residue was purified by flash chromatography (ISCO, 12 g silica, 0-15% MeOH (+10% NH₄OH)/DCM) to afford an off-white solid (32 mg, 38%). The racemate was separated by chiral-SFC (REGIS (S,S) WHELK-01, 250×25 mm, 10 μm); 50% MeOH (0.1% NH₄OH) in CO₂) to afford:

Peak 1, Example 215. (R)—N-(5-(1,4-oxazepan-5-yl)pyridin-3-yl)-4-amino-1-(2,6-dichlorophenyl)-6-oxo-1,6-dihydropyrimidine-5-carboxamide or (S)—N-(5-(1,4-oxazepan-5-yl)pyridin-3-yl)-4-amino-1-(2,6-dichlorophenyl)-6-oxo-1,6-dihydropyrimidine-5-carboxamide (off-white solid, 9.9 mg, 30%). LCMS m/z=475 [M+H]⁺; ¹H NMR (400 MHz, CDCl₃): 11.66 (s, 1H), 9.98 (br d, 1H), 8.61 (br s, 1H), 8.27 (br s, 1H), 8.11 (s, 1H), 7.66 (s, 1H), 7.49-7.47 (m, 2H), 7.41-7.39 (m, 1H), 5.99 (br d, 1H), 3.97 (br t, 1H), 3.90-3.70 (m, 4H), 3.11-3.06 (m, 1H), 3.01-2.95 (m, 1H), 2.07-2.04 (m, 2H).

### Example 216. Synthesis of (R)-4-amino-1-(2,6-dimethyl-4-(trifluoromethyl)phenyl)-6-oxo-N-(5-(piperidin-2-yl)pyridin-3-yl)-1,6-dihydropyrimidine-5-carboxamide

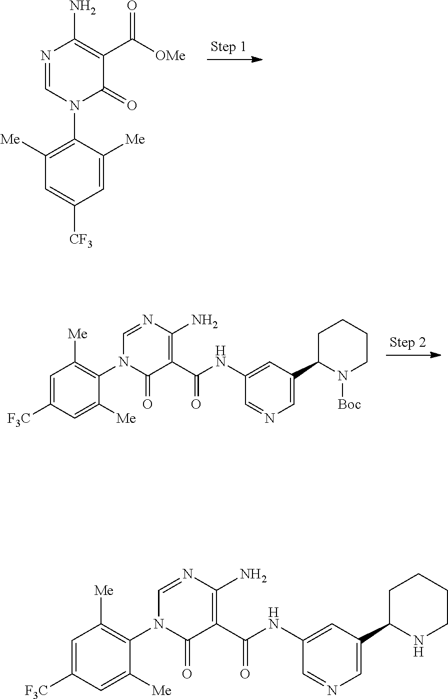 [SMILES: COC(=O)c1c(N)ncn(-c2c(C)cc(C(F)(F)F)cc2C)c1=O ; Cc1cc(C(F)(F)F)cc(C)c1-n1cnc(N)c(C(=O)Nc2cncc([C@H]3CCCCN3C(=O)OC(C)(C)C)c2)c1=O ; Cc1cc(C(F)(F)F)cc(C)c1-n1cnc(N)c(C(=O)Nc2cncc([C@H]3CCCCN3)c2)c1=O]

### Step 1. Synthesis of tert-butyl(R)-2-(5-(4-amino-1-(2,6-dimethyl-4-(trifluoromethyl)phenyl)-6-oxo-1,6-dihydropyrimidine-5-carboxamido)pyridin-3-yl) piperidine-1-carboxylate

To a mixture of methyl 4-amino-1-(2,6-dimethyl-4-(trifluoromethyl)phenyl)-6-oxo-1,6-dihydropyrimidine-5-carboxylate (Intermediate B24, 1.1g 3.22 mmol) and tert-butyl(R)-2-(5-amino-3-pyridyl) piperidine-1-carboxylate (Intermediate A4, 983 mg, 3.55 mmol) in toluene (20 mL) was added AlMe₃ (2M in toluene, 4.83 mL) at 0° C. under N₂. The reaction mixture was stirred at 100° C. for 1 h under N₂. The reaction mixture was quenched with 2M NaOH (50 ml) and extracted with EtOAc (3×60 mL). The combined organics were dried (Na₂SO₄) and evaporated to dryness in vacuo. The residue was purified by column chromatography on silica gel (0-50% EtOAc/PE) to afford the title compound as a brown solid (1 g, 53%). LCMS m/z=587 [M+H]⁺.

### Step 2. Synthesis of (R)-4-amino-1-(2,6-dimethyl-4-(trifluoromethyl)phenyl)-6-oxo-N-(5-(piperidin-2-yl)pyridin-3-yl)-1,6-dihydropyrimidine-5-carboxamide

A mixture of tert-butyl(R)-2-(5-(4-amino-1-(2,6-dimethyl-4-(trifluoromethyl)phenyl)-6-oxo-1,6-dihydropyrimidine-5-carboxamido)pyridin-3-yl) piperidine-1-carboxylate (Step 1, 800 mg, 1.36 mmol) in HCl/EtOAc (2 mL) and EtOAc (4 mL) was stirred at 25° C. for 1 h. The reaction mixture was evaporated to dryness in vacuo and the pH of the residue adjusted to pH 9-10 with 0.25 M aqueous Na₂CO₃ solution. The solids were collected to afford the title compound as a white solid (560 mg, 84%). LCMS m/z=487 [M+H]⁺; ¹H NMR (400 MHz, CDCl₃): 11.77 (s, 1H), 9.95 (d, 1H), 8.60 (d, 1H), 8.23 (d, 1H), 8.08 (t, 1H), 7.63 (s, 1H), 7.44 (s, 2H), 6.12 (d, 1H), 3.52-3.61 (m, 1H), 3.13 (d, 1H), 2.64-2.76 (m, 1H), 2.18 (s, 6H), 1.72-1.86 (m, 2H), 1.55-1.61 (m, 1H), 1.45-1.51 (m, 1H), 1.36-1.45 (m, 2H).

### Example 217. Synthesis of (S)-4-amino-1-((S)-2-chloro-6-methylphenyl)-N-(2-fluoro-3-(pyrrolidin-2-yl)phenyl)-6-oxo-1,6-dihydropyrimidine-5-carboxamide or (R)-4-amino-1-((S)-2-chloro-6-methylphenyl)-N-(2-fluoro-3-(pyrrolidin-2-yl)phenyl)-6-oxo-1,6-dihydropyrimidine-5-carboxamide

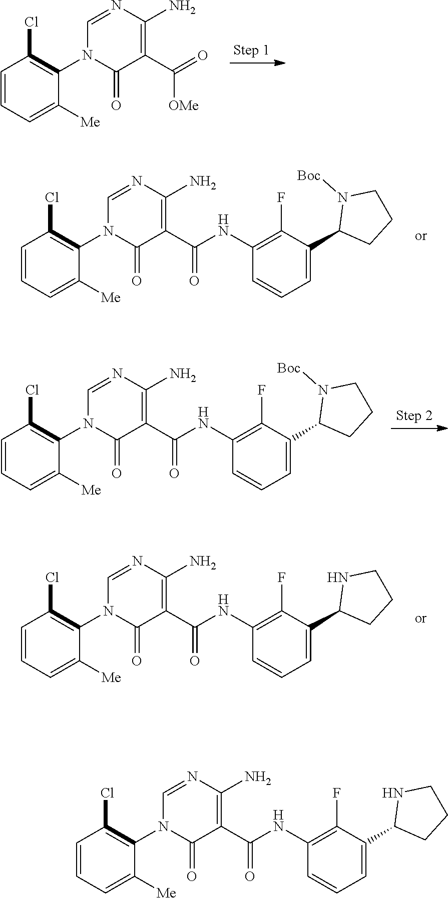

### Step 1. Synthesis of tert-butyl(S)-2-(3-(4-amino-1-((S)-2-chloro-6-methylphenyl)-6-oxo-1,6-dihydropyrimidine-5-carboxamido)-2-fluorophenyl) pyrrolidine-1-carboxylate or tert-butyl(R)-2-(3-(4-amino-1-((S)-2-chloro-6-methylphenyl)-6-oxo-1,6-dihydropyrimidine-5-carboxamido)-2-fluorophenyl) pyrrolidine-1-carboxylate

To the mixture of methyl 4-amino-1-((S)-2-chloro-6-methylphenyl)-6-oxo-1,6-dihydropyrimidine-5-carboxylate (Intermediate B3, 100 mg, 0.340 mmol) and tert-butyl(S)-2-(3-amino-2-fluorophenyl) pyrrolidine-1-carboxylate or tert-butyl(R)-2-(3-amino-2-fluorophenyl) pyrrolidine-1-carboxylate (Intermediate A85, 124 mg, 0.443 mmol) in toluene (1.5 mL) was added AlMe₃ (2 M, 0.51 mL) in toluene at 0° C. under N₂ and the mixture stirred at 100° C. for 0.5 h under N₂. The mixture reaction was quenched by adding into water (10 mL). The reaction mixture was concentrated under reduced pressure and the residue partitioned between DCM (20 mL) and aq. 1 M NaOH (15 mL) and the aqueous phase extracted with DCM (3×20 mL). The combined organics were dried (Na₂SO₄) and concentrated under reduced pressure and the residue purified by prep-TLC (SiO₂, 50% EtOAc/PE). The residue was further purified by chiral SFC (Diacel Chiralpak AD, 30×250 mm, 10 μm; 33% IPA (0.1% NH₄OH) in CO₂) to afford:

Peak 1, tert-butyl(S)-2-(3-(4-amino-1-((S)-2-chloro-6-methylphenyl)-6-oxo-1,6-dihydropyrimidine-5-carboxamido)-2-fluorophenyl) pyrrolidine-1-carboxylate or tert-butyl(R)-2-(3-(4-amino-1-((S)-2-chloro-6-methylphenyl)-6-oxo-1,6-dihydropyrimidine-5-carboxamido)-2-fluorophenyl) pyrrolidine-1-carboxylate (40 mg, 22%). LCMS m/z=564 [M+Na]⁺.

### Step 2. Synthesis of (S)-4-amino-1-((S)-2-chloro-6-methylphenyl)-N-(2-fluoro-3-(pyrrolidin-2-yl)phenyl)-6-oxo-1,6-dihydropyrimidine-5-carboxamide or (R)-4-amino-1-((S)-2-chloro-6-methylphenyl)-N-(2-fluoro-3-(pyrrolidin-2-yl)phenyl)-6-oxo-1,6-dihydropyrimidine-5-carboxamide

tert-butyl(S)-2-(3-(4-amino-1-((S)-2-chloro-6-methylphenyl)-6-oxo-1,6-dihydropyrimidine-5-carboxamido)-2-fluorophenyl) pyrrolidine-1-carboxylate or tert-butyl(R)-2-(3-(4-amino-1-((S)-2-chloro-6-methylphenyl)-6-oxo-1,6-dihydropyrimidine-5-carboxamido)-2-fluorophenyl) pyrrolidine-1-carboxylate (Step 1, 40 mg, 0.074 mmol) in HCl/EtOAc (2 mL) was stirred at 20° C. for 1 h. The mixture was concentrated under a flow of N₂ and the residue purified by prep-HPLC-3 (10-50% MeCN) to give the title compound as a yellow solid (10.1 mg, 31%). LCMS m/z=442 [M+H]⁺; ¹H NMR (400 MHz, DMSO-d₆): 12.10 (d, 1H), 9.61 (d, 1H), 8.62 (d, 1H), 8.32 (s, 1H), 8.27-8.24 (m, 1H), 7.58-7.56 (m, 1H), 7.52-7.45 (m, 2H), 7.25-7.22 (m, 1H), 7.14-7.10 (m, 1H), 4.34 (br t, 1H), 3.04-2.91 (m, 2H), 2.21-2.15 (m, 4H), 1.79-1.74 (m, 2H), 1.56-1.49 (m, 1H).

### Example 218. Synthesis of (R)-4-amino-N-(5-(1-aminoethyl)pyridin-3-yl)-1-(4-ethyl-2,6-dimethylphenyl)-6-oxo-1,6-dihydropyrimidine-5-carboxamide or (S)-4-amino-N-(5-(1-aminoethyl)pyridin-3-yl)-1-(4-ethyl-2,6-dimethylphenyl)-6-oxo-1,6-dihydropyrimidine-5-carboxamide

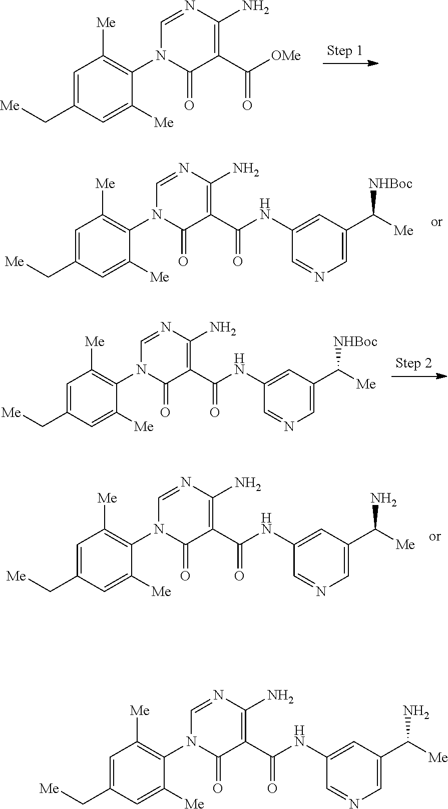 [SMILES: CCc1cc(C)c(-n2cnc(N)c(C(=O)OC)c2=O)c(C)c1 ; CCc1cc(C)c(-n2cnc(N)c(C(=O)Nc3cncc([C@H](C)NC(=O)OC(C)(C)C)c3)c2=O)c(C)c1 ; CCc1cc(C)c(-n2cnc(N)c(C(=O)Nc3cncc([C@@H](C)NC(=O)OC(C)(C)C)c3)c2=O)c(C)c1 ; CCc1cc(C)c(-n2cnc(N)c(C(=O)Nc3cncc([C@H](C)N)c3)c2=O)c(C)c1 ; CCc1cc(C)c(-n2cnc(N)c(C(=O)Nc3cncc([C@@H](C)N)c3)c2=O)c(C)c1]

### Step 1. Synthesis of tert-butyl (R)-(1-(5-(4-amino-1-(4-ethyl-2,6-dimethylphenyl)-6-oxo-1,6-dihydropyrimidine-5-carboxamido)pyridin-3-yl)ethyl)carbamate or tert-butyl(S)-(1-(5-(4-amino-1-(4-ethyl-2,6-dimethylphenyl)-6-oxo-1,6-dihydropyrimidine-5-carboxamido)pyridin-3-yl)ethyl)carbamate

To a mixture of methyl 4-amino-1-(4-ethyl-2,6-dimethylphenyl)-6-oxo-1,6-dihydropyrimidine-5-carboxylate (Intermediate B58, 30 mg, 0.100 mmol) and tert-butyl(S)-(1-(5-aminopyridin-3-yl)ethyl)carbamate or tert-butyl (R)-(1-(5-aminopyridin-3-yl)ethyl)carbamate BP1491256_A6 (Intermediate A16, 30.71 mg, 0.129 mmol) in toluene (3 mL) was added AlMe₃ (2 M in toluene, 0.149.33 mL) at 0° C. and the mixture stirred at 0° C. for 5 min and at 40° C. for 40 min under N₂. To the reaction mixture was added NaOH (2 M, 3 mL) at 0° C. and then extracted with EtOAc (3×10 mL). The combined organics were dried (Na₂SO₄) and concentrated under reduced pressure to give the title compound as a yellow solid (40 mg, 79%). LCMS m/z=507 [M+H]⁺.

### Step 2. Synthesis of (S)-4-amino-N-(5-(1-aminoethyl)pyridin-3-yl)-1-(4-ethyl-2,6-dimethylphenyl)-6-oxo-1,6-dihydropyrimidine-5-carboxamide or (R)-4-amino-N-(5-(1-aminoethyl)pyridin-3-yl)-1-(4-ethyl-2,6-dimethylphenyl)-6-oxo-1,6-dihydropyrimidine-5-carboxamide

To a solution of tert-butyl(S)-(1-(5-(4-amino-1-(4-ethyl-2,6-dimethylphenyl)-6-oxo-1,6-dihydropyrimidine-5-carboxamido)pyridin-3-yl)ethyl)carbamate or tert-butyl (R)-(1-(5-(4-amino-1-(4-ethyl-2,6-dimethylphenyl)-6-oxo-1,6-dihydropyrimidine-5-carboxamido)pyridin-3-yl)ethyl)carbamate (Step 1, 35 mg, 0.069 mmol) in DCM (2 mL) was added TFA (2 mL) and the mixture stirred at 25° C. for 20 min. The reaction mixture was concentrated under N₂ and the residue purified by prep-HPLC-1 (10-45% MeCN) to give the title compound as a white solid (21.5 mg, 76%). LCMS m/z=407 [M+H]⁺; ¹H NMR (400 MHz, CDCl₃): 11.99 (s, 1H), 9.75 (d, 1H), 8.49 (d, 1H), 8.23 (d, 2H), 7.66 (s, 1H), 6.97 (s, 2H), 6.13 (d, 1H), 4.19 (d, 1H), 2.57 (q, 2H), 2.06 (d, 6H), 1.43 (d, 3H), 1.19 (t, 3H).

### Example 219. Synthesis of (R)-4-amino-N-(6-(1-aminoethyl) pyrazin-2-yl)-1-(2,6-dichloro-4-ethoxyphenyl)-6-oxo-1,6-dihydropyrimidine-5-carboxamide or (S)-4-amino-N-(6-(1-aminoethyl) pyrazin-2-yl)-1-(2,6-dichloro-4-ethoxyphenyl)-6-oxo-1,6-dihydropyrimidine-5-carboxamide

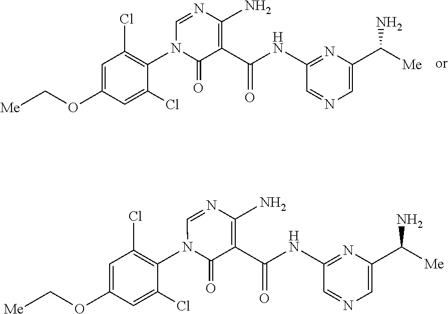 [SMILES: CCOc1cc(Cl)c(-n2cnc(N)c(C(=O)Nc3cncc([C@@H](C)N)n3)c2=O)c(Cl)c1 ; CCOc1cc(Cl)c(-n2cnc(N)c(C(=O)Nc3cncc([C@H](C)N)n3)c2=O)c(Cl)c1]

The title compound was prepared as a yellow solid (16.3 mg, 23%) from methyl 4-amino-1-(2,6-dichloro-4-ethoxyphenyl)-6-oxo-1,6-dihydropyrimidine-5-carboxylate (Intermediate B30) and 6-(1-aminoethyl) pyrazin-2-amine (Intermediate A80) using an analogous method to that described for Example 2. HPLC-1 (10-40% MeCN). LCMS m/z=464 [M+H]⁺; 1H NMR (400 MHz, CDCl₃): 12.11 (s, 1H) 9.97 (s, 1H) 9.51 (s, 1H) 8.27 (s, 1H) 7.75 (s, 1H) 7.04 (s, 2H) 6.06 (s, 1H) 4.22 (m, 1H) 4.09 (m, 2H) 1.44-1.49 (m, 6H).

### Example 220. Synthesis of 4-amino-N-(3-(aminomethyl)-2-ethoxyphenyl)-1-(2,6-dichlorophenyl)-6-oxo-1,6-dihydropyrimidine-5-carboxamide

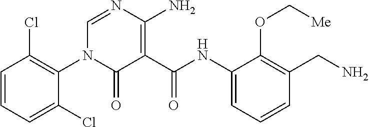 [SMILES: CCOc1c(CN)cccc1NC(=O)c1c(N)ncn(-c2c(Cl)cccc2Cl)c1=O]

The title compound was prepared as a yellow solid (16.3 mg, 23%) from methyl 4-amino-1-(2,6-dichlorophenyl)-6-oxo-1,6-dihydropyrimidine-5-carboxylate (Intermediate B9) and 3-(aminomethyl)-2-ethoxyaniline (Intermediate A83) using an analogous method to that described for Example 2. HPLC-12 (10-50% MeCN); LCMS m/z=448 [M+H]⁺; ¹H NMR (500 MHz, DMSO-d₆): 12.03 (s, 1H), 9.68 (d, 1H), 8.67 (d, 1H), 8.43 (s, 1H), 8.35 (dd, 1H), 8.08 (s, 3H), 7.77 (d, 2H), 7.64 (t, 1H), 7.24-7.13 (m, 2H), 4.03 (s, 2H), 3.81 (q, 2H), 1.26 (t, 3H).

### Example 221. Synthesis of 4-amino-N-(3-(aminomethyl)-2-methoxyphenyl)-1-(2,6-dichlorophenyl)-6-oxo-1,6-dihydropyrimidine-5-carboxamide trifluoroacetate

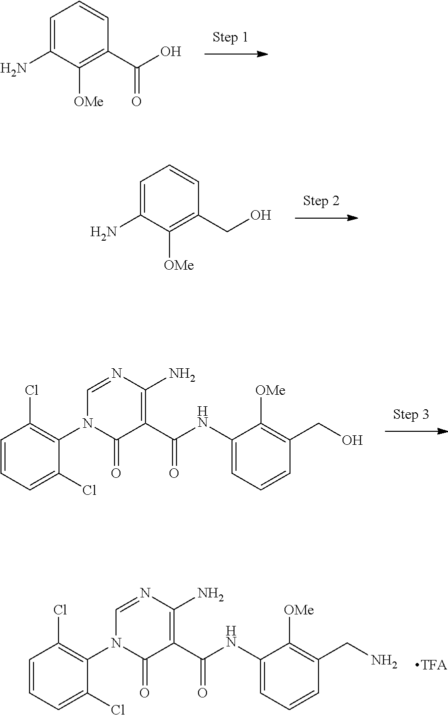 [SMILES: COc1c(N)cccc1C(=O)O ; COc1c(N)cccc1CO ; COc1c(CO)cccc1NC(=O)c1c(N)ncn(-c2c(Cl)cccc2Cl)c1=O ; COc1c(CN)cccc1NC(=O)c1c(N)ncn(-c2c(Cl)cccc2Cl)c1=O]

### Step 1. Synthesis of (3-amino-2-methoxyphenyl) methanol

Anhydrous THF (20 mL) was added to LiAlH₄ (484 mg, 12.5 mmol) under N₂ and the mixture cooled on ice. To this was added dropwise over 15 minutes a suspension of 3-amino-2-methoxybenzoic acid (830 mg, 5.0 mmol) in anhydrous THF (30 mL) and the reaction mixture warmed to RT and heated to 60° C. for 3 h. The reaction was carefully quenched by dropwise addition of water. The mixture was diluted with EtOAc (10 mL) and saturated aqueous NH₄Cl (1 mL) and stirred at RT for 1.5 h. The mixture was poured into aqueous NaHCO₃ and extracted with EtOAc (4×50 mL). The combined organics were washed with water (2×), brine, dried (MgSO₄) and concentrated under reduced pressure. The reside was purified by column chromatography (ISCO 40 g silica, 0-20% MeOH/DCM) to afford the title compound as a waxy solid (466 mg, 60%). LCMS m/z=154 [M+H]⁺.

### Step 2. Synthesis of 4-amino-1-(2,6-dichlorophenyl)-N-(3-(hydroxymethyl)-2-methoxyphenyl)-6-oxo-1,6-dihydropyrimidine-5-carboxamide trifluoroacetate

Trimethylaluminum solution (2.0 M in toluene, 0.450 mmol) was added dropwise to an ice-cold mixture of methyl 4-amino-1-(2,6-dichlorophenyl)-6-oxo-1,6-dihydropyrimidine-5-carboxylate (Intermediate B9, 49.1 mg, 0.150 mmol) and (3-amino-2-methoxyphenyl) methanol (Part 1, 34.5 mg, 0.225 mmol) in dry toluene (3 mL). The reaction mixture was stirred at RT for 10 min and then heated to 100° C. for 75 min. The cooled reaction mixture was diluted with DCM (5 mL) and quenched with MeOH (2 mL) and TFA (0.2 mL). The mixture was poured into water and extracted with DCM (4×10 mL). The combined organics were washed with saturated aqueous sodium bicarbonate, water, brine, dried (MgSO₄) and evaporated to dryness. The residue was purified by prep-HPLC-12 (10-60% MeCN) to afford the title compound as a white powder (26.5 mg, 32%). LCMS m/z=435 [M+H]⁺.

### Step 3. Synthesis of 4-amino-N-(3-(aminomethyl)-2-methoxyphenyl)-1-(2,6-dichlorophenyl)-6-oxo-1,6-dihydropyrimidine-5-carboxamide trifluoroacetate

A solution of 4-amino-1-(2,6-dichlorophenyl)-N-(3-(hydroxymethyl)-2-methoxyphenyl)-6-oxo-1,6-dihydropyrimidine-5-carboxamide trifluoroacetate (Step 2, 24.4 mg, 0.044 mmol), DIPEA (11.5 μL, 0.066 mmol), CBr₄ (24.1 mg, 0.072 mmol) and PPh₃ (19.0 mg, 0.072 mmol) in DMF (0.8 mL) and DMSO (0.2 mL) was stirred at RT for 45 min. To this was added NaN₃ (5.7 mg, 0.088 mmol) and the mixture heated to 60° C. for 3 h. The reaction was diluted with EtOAc (20 mL) and washed with water (3×), brine, dried (MgSO₄) and concentrated by rotary evaporation.

PPh₃ (28.1 mg, 0.107 mmol) and water (20.0 μL, 1.10 mmol) were added to the residue dissolved in THF (0.80 mL) and stirred at RT overnight. The reaction mixture was concentrated and the residue purified by prep-HPLC-12 (10-50% MeCN) to afford the title compound as a colourless film (0.7 mg, 2%). LCMS m/z=434 [M+H]⁺; ¹H NMR (400 MHz, DMSO-d₆): 12.05 (s, 1H), 9.70 (d, 1H), 8.70 (d, 1H), 8.48-8.37 (m, 2H), 8.09 (s, 3H), 7.78 (d, 2H), 7.65 (t, 1H), 7.20 (t, 1H), 7.15 (d, 1H), 4.05 (q, 2H), 3.70 (s, 3H).

### Example 222. Synthesis of (R)-4-amino-1-(2,6-dichloro-4-methoxyphenyl)-N-(3-fluoro-5-(piperidin-2-yl)phenyl)-6-oxo-1,6-dihydropyrimidine-5-carboxamide or (S)-4-amino-1-(2,6-dichloro-4-methoxyphenyl)-N-(3-fluoro-5-(piperidin-2-yl)phenyl)-6-oxo-1,6-dihydropyrimidine-5-carboxamide

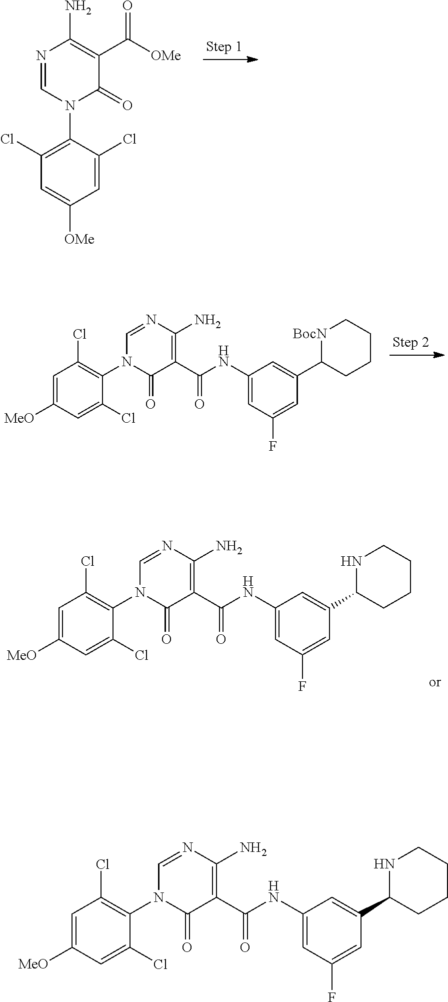 [SMILES: COC(=O)c1c(N)ncn(-c2c(Cl)cc(OC)cc2Cl)c1=O ; COc1cc(Cl)c(-n2cnc(N)c(C(=O)Nc3cc(F)cc(C4CCCCN4C(=O)OC(C)(C)C)c3)c2=O)c(Cl)c1 ; COc1cc(Cl)c(-n2cnc(N)c(C(=O)Nc3cc(F)cc([C@H]4CCCCN4)c3)c2=O)c(Cl)c1 ; COc1cc(Cl)c(-n2cnc(N)c(C(=O)Nc3cc(F)cc([C@@H]4CCCCN4)c3)c2=O)c(Cl)c1]

### Step 1. Synthesis of tert-butyl 2-(3-(4-amino-1-(2,6-dichloro-4-methoxyphenyl)-6-oxo-1,6-dihydropyrimidine-5-carboxamido)-5-fluorophenyl) piperidine-1-carboxylate

Trimethylaluminum solution (2.0 M in toluene, 0.110 mL, 0.214 mmol) was added dropwise to an ice-cold solution of methyl 4-amino-1-(2,6-dichloro-4-methoxyphenyl)-6-oxo-1,6-dihydropyrimidine-5-carboxylate (Intermediate B10, 24.5 mg, 0.071 mmol) and tert-butyl 2-(3-amino-5-fluorophenyl) piperidine-1-carboxylate (Intermediate A85, 33 mg, 0.107 mmol) in dry toluene (1.4 mL). The reaction was stirred at RT for 10 min and then heated to 100° C. for 1 h. The reaction mixture was cooled, diluted with DCM (5 mL) and quenched with MeOH (2 mL) and TFA (0.2 mL). The mixture was poured into water and extracted with DCM (4×10 mL). The combined organics were washed with saturated aqueous sodium bicarbonate, water, brine, dried (MgSO₄) and evaporated to dryness to afford the title compound. LCMS m/z=628 [M+Na]⁺.

### Step 2. (R)-4-amino-1-(2,6-dichloro-4-methoxyphenyl)-N-(3-fluoro-5-(piperidin-2-yl)phenyl)-6-oxo-1,6-dihydropyrimidine-5-carboxamide or (S)-4-amino-1-(2,6-dichloro-4-methoxyphenyl)-N-(3-fluoro-5-(piperidin-2-yl)phenyl)-6-oxo-1,6-dihydropyrimidine-5-carboxamide

Tert-butyl 2-(3-(4-amino-1-(2,6-dichloro-4-methoxyphenyl)-6-oxo-1,6-dihydropyrimidine-5-carboxamido)-5-fluorophenyl) piperidine-1-carboxylate (Step 1) was dissolved in DCM (2 mL) and TFA (0.5 mL) and stirred at RT for 3 h. The solvents were removed by rotary evaporation and the residue purified by prep-HPLC-12 (10-50% MeCN) followed by chiral SFC (Rilas Technologies, Regis Whelk O-1 (S,S) 21×250 mm, 30% EtOH+0.25% diethylamine in CO₂), to afford 70 mL/min) afforded:

Peak 1, Example 222. (R)-4-amino-1-(2,6-dichloro-4-methoxyphenyl)-N-(3-fluoro-5-(piperidin-2-yl)phenyl)-6-oxo-1,6-dihydropyrimidine-5-carboxamide or (S)-4-amino-1-(2,6-dichloro-4-methoxyphenyl)-N-(3-fluoro-5-(piperidin-2-yl)phenyl)-6-oxo-1,6-dihydropyrimidine-5-carboxamide (white solid, 6.3 mg). LCMS m/z=506 [M+H]⁺; 1H NMR (500 MHz, DMSO-d₆): 12.16 (s, 1H), 9.57 (s, 1H), 8.90 (s, 1H), 8.77 (s, 1H), 8.56 (s, 1H), 8.43 (s, 1H), 7.85 (d, 1H), 7.37 (s, 2H), 7.35 (s, 1H), 7.02 (d, 1H), 4.26-4.17 (m, 1H), 3.89 (s, 3H), 3.07-2.95 (m, 1H), 1.97-1.52 (m, 6H).

### Biological Example 1. In Vitro Enzymatic Activity Assay

Full length human GSK3α or GSK3B expressed as N-terminal GST fusion proteins in baculovirus host were purchased from Carna Biosciences (Japan). The kinase was pre-incubated with ATP (adenosine-5′-triphosphate) for 45 min, then incubated with tested compounds for another 45 min in assay buffer that contains 100 mM HEPES (N-2-hydroxyethylpiperazine-N′-2-ethanesulfonic acid), pH 7.5, 10 mM MgCl₂, 1.0 mM DTT, and 0.015% Briji-35. Serial dilution of compound was prepared in DMSO (dimethyl sulfoxide and transferred to 384-well plates (Greiner BioOne) by Mosquito (SPT Labtech). The final reaction mixture containing 0.2 nM GSK3α or GSK3B kinase, 1.0 mM ATP (measured Km for ATP is 10-15 μM), and 1.0 μM peptide substrate (5-FAM-KRREILSRRPpSYR—COOH (SEQ ID NO: 1), ProfilerPro Peptide 15, Perkin Elmer) was initiated by addition of substrate peptide and incubated for 120 minutes at 25° C. Reaction was terminated by the addition of excess EDTA. Substrate and product were separated, and fluorescence intensities of the substrate and product peaks were determined by Labchip EZ Reader II (Caliper Life Sciences, Hopkinton, MA). Percent inhibition was plotted against compound concentration to generate 10-point dose response curve with 3-fold serial dilution starting from 10 μM. IC₅₀ value was determined from the 4-parameter logistic curve fitting.

### Biological Example 2. In Vitro Cellular Activity Assay

Cellular target engagement, or cellular binding of the testing compounds were measured in NanoBRET™ assays, which is based on binding competition between the testing compounds and a bioluminescent tracer in human embryonic kidney cells (HEK-293 cell line). In the assay, HEK293 cells stably expressing either Nanoluc-GSK3A or Nanoluc-GSK3B were maintained in culture media and seeded, using a multidrop dispenser, at a density of 5×10³ cells per well in 384-well white plates with 100 ng/ml doxycycline (Sigma) overnight.

Compound was first diluted in DMSO to generate a 9-point serial dilution then diluted to 10× final concentration in Opti-MEM™ (Gibco). After overnight incubation, cell medium was aspirated from the seeded cells using a microplate automatic washer, then 27 μL of pre-warmed Opti-MEM™ with kinase tracer (K-8) and 3 μL of Opti-MEM™ diluted DMSO or compounds were added per well. Plates were protected from light, mixed in an orbital shaker for 3 min at RT and incubated at 37° C., 5% CO₂ in a humidified incubator for 2 hours. After acclimation at RT for 15 min, 15 μL of 3× complete NanoBRET™ Nano-Glo Substrate/Inhibitor (Promega) was added per well. Plates were mixed in an orbital shaker for 3 min at RT and incubated for an additional 30 min. Donor emission at 460 nm and acceptor emission at 647 nm were measured in EnVision multilabel plate reader (Perkin Elmer).

Biological assay data of exemplary compounds are provided in Table 1 below.

TABLE 1

| | | | NanoBRET | NanoBRET |
|---|---|---|---|---|
| | Enzyme | Enzyme | GSK3α | GSK3β |
| | GSK3α | GSK3β | IC50 | IC50 |
| EXAMPLE | (nM) | (nM) | (nM) | (nM) |
| 1 | 3.6 | 167.9 | 3.6 | 243 |
| 2 | 7 | 399.4 | 4.9 | 328.9 |
| 3 | 6.6 | 344.4 | 5 | 395.2 |
| 4 | 3.7 | 186.8 | 1.4 | 109.3 |
| 5 | 8.3 | 378.1 | 6.2 | 382.7 |
| 6 | 7.2 | 292.4 | 6.5 | 352 |
| 7 | 7.5 | 336.8 | 7.1 | 480.3 |
| 8 | 4.8 | 204.3 | 3.3 | 169.1 |
| 9 | 9.5 | 661.3 | 7.4 | 429.9 |
| 10 | 8.8 | 395 | 6.9 | 370.5 |
| 11 | 14.4 | 647.6 | 4.1 | 252.2 |
| 12 | 3.8 | 199.2 | 2.4 | 140.8 |
| 13 | 17.7 | 1006.8 | 9.8 | 502.6 |
| 14 | 6.4 | 318.4 | 4 | 230.7 |
| 15 | 4.5 | 208 | 2.2 | 118.6 |
| 16 | 4.1 | 254.7 | 1.9 | 132.3 |
| 17 | 7.8 | 339.6 | 3.8 | 270.6 |
| 18 | 11.9 | 621.5 | 7.1 | 428.9 |
| 19 | 3.2 | 231.7 | 3.5 | 179.8 |
| 20 | 14 | 685.1 | 7.2 | 396.5 |
| 21 | 3.2 | 216.6 | 2.6 | 145.2 |
| 22 | 8.4 | 389.3 | 5.6 | 334.1 |
| 23 | 5 | 342.7 | 4.3 | 239.3 |
| 24 | 4.1 | 297.8 | 2.8 | 146.9 |
| 25 | 13.9 | 691.8 | 7.5 | 714.9 |
| 26 | 5.5 | 327.2 | 4.4 | 268.7 |
| 27 | 2.4 | 54.2 | 1.7 | 42.5 |
| 28 | 9.6 | 320.4 | 9 | 698.8 |
| 29 | 92.2 | 3799.7 | 79.2 | 25000 |
| 30 | 63.9 | 1949 | 72.2 | 15760.8 |
| 31 | 6.9 | 404.8 | 5 | 417.8 |
| 32 | 6.3 | 269 | 2.6 | 157.8 |
| 33 | 6.4 | 343.1 | 8.3 | 460.7 |
| 34 | 6.5 | 344.4 | 3.7 | 268.1 |
| 35 | 14.7 | 693.4 | 6.2 | 411.6 |
| 36 | 7.3 | 301.9 | 4.4 | 289.8 |
| 37 | 8.4 | 388 | 7.6 | 431.4 |
| 38 | 4.6 | 282.3 | 3.6 | 229.9 |
| 39 | 2.6 | 106.9 | 2.1 | 128.6 |
| 40 | 10.1 | 418 | 5.8 | 430.5 |
| 41 | 12.1 | 604.8 | 5.9 | 385.9 |
| 42 | 4.8 | 297.5 | 2.9 | 257.1 |
| 43 | 4.9 | 283 | 5.4 | 284.7 |
| 44 | 4.7 | 381.8 | 7.6 | 518 |
| 45 | 8.5 | 384.4 | 6.6 | 364.7 |
| 46 | 10.5 | 489.3 | 7.3 | 395 |
| 47 | 3.2 | 227.3 | 2.9 | 173.9 |
| 48 | 5.2 | 343.4 | 4.7 | 297.9 |
| 49 | 2.5 | 160.6 | 2.8 | 144.5 |
| 50 | 16.5 | 708.3 | 7.4 | 438.9 |
| 51 | 2.8 | 141.9 | 2.2 | 123.5 |
| 52 | 4.4 | 208.8 | 2.5 | 131.5 |
| 53 | 12.9 | 625.3 | 6.8 | 356.2 |
| 54 | 5.4 | 345.2 | 5.4 | 340.3 |
| 55 | 9.1 | 430.1 | 6.5 | 483.3 |
| 56 | 5.7 | 323.4 | 4.8 | 272.8 |
| 57 | 7.9 | 382.2 | 5.4 | 276.8 |
| 58 | 2.1 | 152.5 | 2.8 | 146.6 |
| 59 | 3.1 | 224.3 | 2.5 | 187 |
| 60 | 6.5 | 333.8 | 3 | 199.5 |
| 61 | 6.4 | 465.5 | 3.7 | 284.4 |
| 62 | 2 | 123 | 2.4 | 155.4 |
| 63 | 4.5 | 209.4 | 4.4 | 234 |
| 64 | 11.7 | 621.3 | 6.5 | 489.4 |
| 65 | 5.1 | 302 | 3.1 | 217 |
| 66 | 6 | 348.8 | 2.9 | 206 |
| 67 | 2.1 | 105.1 | 1.4 | 73.4 |
| 68 | 2.6 | 167.3 | 1.9 | 219.6 |
| 69 | 12.5 | 536 | 7 | 513.3 |
| 70 | 2.9 | 145.3 | 2.4 | 201.4 |
| 71 | 6.3 | 383.4 | 4.8 | 353 |
| 72 | 7.5 | 468.3 | 5.1 | 378.6 |
| 73 | 6.7 | 318 | 5.8 | 374 |
| 74 | 9.6 | 464.8 | 6.6 | 363.4 |
| 75 | 2.4 | 106.1 | 3.6 | 186.2 |
| 76 | 16 | 854 | 9.4 | 591.4 |
| 77 | 2.2 | 152.1 | 3.5 | 195.9 |
| 78 | 11.4 | 958.4 | 7.7 | 703.4 |
| 79 | 4.3 | 316.9 | 2.4 | 169.8 |
| 80 | 2.5 | 269.9 | 2.9 | 270.7 |
| 81 | 5.9 | 313.7 | 2.3 | 116.6 |
| 82 | 4.5 | 247.6 | 4 | 269.3 |
| 83 | 7 | 490.1 | 4.1 | 336.3 |
| 84 | 3.8 | 253.8 | 2.5 | 179.3 |
| 85 | 9.4 | 551 | 4.2 | 373.8 |
| 86 | 6.6 | 413.7 | 4.2 | 320.9 |
| 87 | 17.9 | 1127.3 | 7.6 | 542.8 |
| 88 | 15.4 | 1015.7 | 8.1 | 453.6 |
| 89 | 10.2 | 668.9 | 6.4 | 484.6 |
| 90 | 15.6 | 1065.2 | 6.5 | 700.1 |
| 91 | 6 | 299.1 | 3.7 | 200.7 |
| 92 | 16.4 | 1027 | 6.8 | 584 |
| 93 | 31.7 | 1949.2 | 9.3 | 914.4 |
| 94 | 7.6 | 598.4 | 6.6 | 383.5 |
| 95 | 9.8 | 464.6 | 4.3 | 289.6 |
| 96 | 16.4 | 705.8 | 6.7 | 640.2 |
| 97 | 3.6 | 229.7 | 1.8 | 120.8 |
| 98 | 4.6 | 253.5 | 2.5 | 133.5 |
| 99 | 20.5 | 1023.6 | 7.4 | 565.2 |
| 100 | 8.2 | 347.8 | 3.9 | 293.1 |
| 101 | 19.6 | 941.3 | 7.8 | 799.7 |
| 102 | 16.5 | 683.8 | 5 | 584 |
| 103 | 13.2 | 581 | 6.6 | 532.5 |
| 104 | 16.2 | 746.2 | 7.9 | 520.9 |
| 105 | 19.3 | 1126.7 | 7.2 | 576.6 |
| 106 | 4.5 | 278.9 | 2.8 | 195.5 |
| 107 | 10 | 671.4 | 3.9 | 434.5 |
| 108 | 24.6 | 998.1 | 8.7 | 688.4 |
| 109 | 15.6 | 802.9 | 8.4 | 567 |
| 110 | 9.9 | 567.8 | 3.7 | 334.9 |
| 111 | 3 | 163.3 | 2 | 139.9 |
| 112 | 10.5 | 493.5 | 5.1 | 348.5 |
| 113 | 3 | 132.9 | 1.7 | 107 |
| 114 | 12.1 | 506.7 | 4.4 | 251.9 |
| 115 | 7.6 | 362.2 | 4.7 | 280.7 |
| 116 | 2.9 | 125.1 | 1.8 | 101.4 |
| 117 | 2.2 | 104.1 | 1.4 | 81.1 |
| 118 | 11.2 | 449.8 | 9.4 | 566.3 |
| 119 | 8.1 | 414.1 | 8.1 | 502.1 |
| 120 | 9.4 | 442.2 | 4.4 | 274.3 |
| 121 | 3.8 | 306.5 | 3.4 | 395.4 |
| 122 | 5.3 | 327 | 5 | 313.8 |
| 123 | 4.8 | 266.2 | 3.3 | 202.6 |
| 124 | 7 | 393.4 | 7.8 | 404.3 |
| 125 | 4.9 | 296.5 | 2.7 | 162.6 |
| 126 | 14.7 | 701.8 | 7.9 | 505.9 |
| 127 | 18.6 | 767.4 | 9.6 | 649.6 |
| 128 | 19 | 1175.6 | 7.7 | 545.9 |
| 129 | 2.1 | 104.8 | 1.2 | 81.7 |
| 130 | 13.5 | 591 | 5.3 | 461.4 |
| 131 | 4.9 | 201.7 | 2.3 | 159 |
| 132 | 5.5 | 228 | 2 | 180.7 |
| 133 | 9.7 | 508.2 | 6.1 | 349.5 |
| 134 | 3.4 | 212.1 | 3.2 | 204 |
| 135 | 3.2 | 184.2 | 4.5 | 264.3 |
| 136 | 2.9 | 205.7 | 1.8 | 118.1 |
| 137 | 1.9 | 129.4 | 1.7 | 87.1 |
| 138 | 6.4 | 321.7 | 2.9 | 211.7 |
| 139 | 10.9 | 559.7 | 5.1 | 281.2 |
| 140 | 24.2 | 1235.7 | 9.2 | 615.8 |
| 141 | 4.1 | 232 | 2.9 | 317.7 |
| 142 | 9.8 | 420.6 | 4.7 | 349.8 |
| 143 | 4.9 | 207.2 | 4.1 | 280.1 |
| 144 | 11 | 472.4 | 5 | 375.4 |
| 145 | 3 | 129.3 | 1 | 96.2 |
| 146 | 7.7 | 355.3 | 3.7 | 339.8 |
| 147 | 8.8 | 392 | 3.8 | 354 |
| 148 | 7.2 | 316.5 | 4.3 | 349 |
| 149 | 13.4 | 552.5 | 8.6 | 518.6 |
| 150 | 1.1 | 44.2 | 1 | 49.5 |
| 151 | 14.9 | 500.9 | 7.1 | 405.8 |
| 152 | 2.7 | 119.8 | 2.4 | 173.6 |
| 153 | 7.8 | 345.8 | 6.4 | 490.9 |
| 154 | 7 | 245.5 | 3.2 | 258.9 |
| 155 | 9.9 | 373.5 | 3.7 | 278.8 |
| 156 | 34.9 | 1252.1 | 8.1 | 956.6 |
| 157 | 32.5 | 1315.6 | 11.3 | 1155 |
| 158 | 9.3 | 355.1 | 37.3 | 531.9 |
| 159 | 90.6 | 2082.4 | 76.9 | 5788.8 |
| 160 | 161.4 | 2316.7 | 196.9 | 11315.6 |
| 161 | 7.2 | 378.8 | 3 | 287.3 |
| 162 | 5.6 | 218.3 | 5.4 | 300.5 |
| 163 | 5.6 | 250 | 2.8 | 205 |
| 164 | 29.4 | 1849.6 | 9.7 | 1165.9 |
| 165 | 10.8 | 434.3 | 9.3 | 1119.1 |
| 166 | 285.1 | 9383.3 | 176.6 | 16079.1 |
| 167 | 45.4 | 1814.1 | 27.2 | 1864 |
| 168 | 49.3 | 1526.6 | 27.1 | 1469.9 |
| 169 | 4.2 | 105.2 | 2 | 103.4 |
| 170 | 21.8 | 520 | 16.4 | 525.6 |
| 171 | 44.8 | 1278.1 | 30.1 | 851.3 |
| 172 | 6.3 | 259.9 | 3.5 | 98.8 |
| 173 | 1883.3 | 10000 | 1510.8 | 25000 |
| 174 | 28 | 705.1 | 8.7 | 559.8 |
| 175 | 32.6 | 803.3 | 8.1 | 580.7 |
| 176 | 5.1 | 272.3 | 2.6 | 190 |
| 177 | 4 | 199.7 | 2.8 | 283 |
| 178 | 7.2 | 335.2 | 4.8 | 259.7 |
| 179 | 3.7 | 256.8 | 3.3 | 178.6 |
| 180 | 6.4 | 297.5 | 4.7 | 321.6 |
| 181 | 7.9 | 535.6 | 7 | 490.7 |
| 182 | 17.4 | 914.6 | 8.2 | 643.1 |
| 183 | 7.9 | 576 | 5 | 299.9 |
| 184 | 3.9 | 175.3 | 2.1 | 166.7 |
| 185 | 3.5 | 147.8 | 1.6 | 128 |
| 186 | 4.4 | 169.2 | 2.1 | 151.9 |
| 187 | 8.4 | 356.6 | 5.6 | 421.1 |
| 188 | 10.4 | 460.4 | 3.9 | 345.1 |
| 189 | 133 | 2164.3 | 69.4 | 4579.9 |
| 190 | 37.6 | 971.5 | 26.7 | 3300.4 |
| 191 | 5.7 | 229.5 | 4.7 | 304.3 |
| 192 | 1.8 | 14.7 | 1.4 | 18.8 |
| 193 | 9.2 | 480.6 | 6.8 | 487.1 |
| 194 | 6.2 | 302.2 | 5.1 | 329.8 |
| 195 | 11 | 632.1 | 5.4 | 462.5 |
| 196 | 6.6 | 399.7 | 4.2 | 242.2 |
| 197 | 15 | 849.5 | 7.2 | 402.6 |
| 198 | 5.6 | 331.5 | 3.7 | 208.8 |
| 199 | 3.3 | 202.7 | 2.4 | 118 |
| 200 | 14.4 | 813.5 | 7.6 | 451.1 |
| 201 | 13.7 | 607.8 | 7.4 | 430.2 |
| 202 | 8.7 | 431.6 | 5.9 | 385.4 |
| 203 | 8 | 318.1 | 3.7 | 293.2 |
| 204 | 13.5 | 706.3 | 8.8 | 781.9 |
| 205 | 5 | 238 | 3.7 | 204.5 |
| 206 | 9.4 | 259.8 | 3.1 | 139.1 |
| 207 | 5.3 | 233.8 | 965.5 | 25000 |
| 208 | 2.4 | 203 | 2.4 | 166.2 |
| 209 | 5.2 | 383 | 5.9 | 300.4 |
| 210 | 3.9 | 204.5 | 4 | 227.7 |
| 211 | 15.9 | 887.5 | 8.6 | 797.4 |
| 212 | 4.7 | 272.2 | 7.6 | 519.8 |
| 213 | 33.2 | 1759.9 | 7.8 | 992.2 |
| 214 | 17.3 | 930 | 8.9 | 640 |
| 215 | 7.2 | 500.3 | 8.5 | 689.9 |
| 216 | 5.6 | 292.6 | 3.4 | 172.8 |
| 217 | 7.8 | 305 | 8 | 424.3 |
| 218 | 10.8 | 420.6 | 5.6 | 401.3 |
| 219 | 5.4 | 270.6 | 3.8 | 187.4 |
| 220 | 11.7 | 307.8 | 8.1 | 258.6 |
| 221 | 15.6 | 399.9 | 7.3 | 235.5 |
| 222 | 2.1 | 99.2 | 4.6 | 147.6 |

### Biological Example 3. Efficacy in MC38 Syngeneic Colorectal Cancer Model

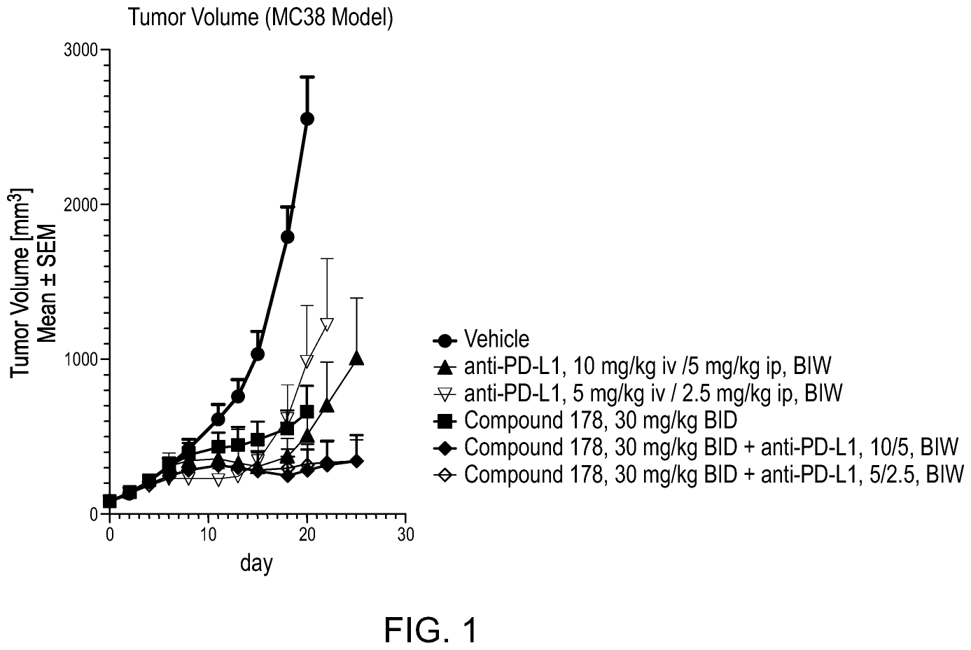

Murine MC38 colorectal carcinoma cells were cultured in DMEM medium supplemented with 10% fetal bovine serum, 100 U/mL Penicillin and 100 μg/mL Streptomycin at 37 C in an atmosphere of 5% CO₂. Cells with viability >90% were harvested and counted for subcutaneous inoculation. Female C₅₇BL/6 mice, 8-9 weeks old, (Beijing Vital River Laboratory Animal Technology Co), weighing approximately 18-22g, were acclimated for 1 week and then inoculated subcutaneously with MC38 cell suspension (1×10⁶ cells) in 100 μL PBS into the right flank. Body weight of individual mice and tumor volume were used as randomization parameter for allocating of animals into treatment groups. Treatment started when tumor size reached an average of 90 mm³ (range 80-100 mm³). Compound in vehicle (20% (w/w) HP-β-CD in 50 mM citrate buffer, pH 3) was prepared fresh daily and administered via oral gavage. Anti-PD-L1 injection solution was administered twice weekly by an initial intravenous loading dose of 5 mg/kg followed by intraperitoneal maintenance doses of 2.5 mg/kg BIW. Animals were monitored daily for clinical symptoms and detection of adverse effects. The volume of engrafted MC38 tumors was measured twice weekly using caliper. Tumor and body weight measurements were recorded for analysis. FIG. 1 shows tumor volume in an MC38 mouse model of colorectal cancer with treatment with anti-PD-L1 antibody, Compound 178 or a combination of anti-PD-L1 antibody and Compound 178.

What is claimed is:

1. A compound of Formula (I), 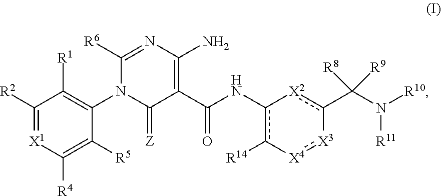
or a pharmaceutically acceptable salt thereof,
wherein [symbol]
is a single bond or a double bond;
X¹ is CR³ or N;
X² is selected from the group consisting of CR⁷, N, and NR^{d};
X³ is selected from the group consisting of CR¹², N, and NR^{d},
X⁴ is selected from the group consisting of CR¹³, N, and NR^{d};
Z is O or S;
R¹ is selected from the group consisting of halo, OH, CN, C₁-C₄alkyl, and C₁-C₄alkoxy, wherein the C₁-C₄alkyl and C₁-C₄alkoxy are each optionally substituted with 1 to 4 groups each independently selected from the group consisting of halo, OH and CN;
R² is selected from the group consisting of H, D, halo, C₁-C₄alkyl, and C₃-C₁₀cycloalkyl;
R³ is selected from the group consisting of H, D, halo, CN, C₁-C₄alkyl, —(C(R^{a})₂)ₙ—OR^{b}, —(C(R^{a})₂)ₙ—C(O)OR^{b}, —(C(R^{a})₂)ₙ—SO₂—R^{b}, —N(R^{a})₂, C₃-C₁₀cycloalkyl, 4 to 12-membered heterocyclyl, 4 to 12-membered aryl, and 4 to 12-membered heteroaryl, wherein the C₁-C₄alkyl, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c};
R⁴ is selected from the group consisting of H, D, halo, C₁-C₄alkyl, and C₃-C₁₀cycloalkyl;
R⁵ is selected from the group consisting of halo, OH, CN, C₁-C₄alkyl, and C₁-C₄alkoxy, wherein the C₁-C₄alkyl and C₁-C₄alkoxy each optionally substituted with 1 to 4 groups each independently selected from the group consisting of halo, OH and CN;
R⁶ is H or D;
R⁷ is selected from the group consisting of H, D, halo, C₁-C₄alkyl, C₁-C₄alkoxy, C₃-C₁₀cycloalkyl, 4 to 12-membered heterocyclyl, 4 to 12-membered aryl, and 4 to 12-membered heteroaryl, wherein the C₁-C₄alkyl, C₁-C₄alkoxy, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c};
R⁸ is selected from the group consisting of H, D, halo, C₁-C₄alkyl, and C₃-C₁₀cycloalkyl;
R⁹ are each independently selected from the group consisting of H, D, C₁-C₄alkyl, C₂-C₄alkenyl, C₂-C₄alkynyl, —(C(R^{a})₂)ₙ—OR^{b}, C₃-C₁₀cycloalkyl, 4 to 12-membered heterocyclyl, 4 to 12-membered aryl, and 4 to 12-membered heteroaryl, wherein the C₁-C₄alkyl, C₂-C₄alkenyl, C₂-C₄alkynyl, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c}; or
R⁷ and R⁹ are taken together with the carbon atoms to which they are attached to form Ring A, wherein Ring A is C₃-C₁₀cycloalkyl or 4 to 12-membered heterocyclyl, wherein the C₃-C₁₀cycloalkyl is optionally substituted with 1 to 4 R^{c}, wherein the 4 to 12-membered heterocyclyl has 1 to 4 ring heteroatoms each independently selected from the group consisting of O, S, and NR^{d}, and then is optionally substituted on a ring carbon by 1 to 4 R^{c}; or
R⁸ and R⁹ are taken together with the carbon atom to which they are attached to form Ring B, wherein Ring B is C₃-C₁₀cycloalkyl or 4 to 12-membered heterocyclyl, wherein the C₃-C₁₀cycloalkyl is optionally substituted with 1 to 4 R^{c}, wherein the 4 to 12-membered heterocyclyl has 1 to 4 ring heteroatoms each independently selected from the group consisting of O, S, and NR⁴, and then is optionally substituted on a ring carbon by 1 to 4 R^{c}; or
R⁸ and R⁹ are taken together form a ═O;
R¹⁰ is selected from the group consisting of H, D, C₁-C₄ alkyl, —(C(R^{a})₂)ₙ—OR^{b}, —(C(R^{a})₂)ₙ—SO₂R^{b}, C₃-C₁₀cycloalkyl, 4 to 12-membered heterocyclyl, 4 to 12-membered aryl, and 4 to 12-membered heteroaryl, wherein the C₁-C₄alkyl, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c}; or
R⁹ and R¹⁰ are taken together with the carbon atom and the nitrogen atom to which they are attached, respectively, to form Ring C, wherein Ring C is a 4 to 12-membered heterocyclyl having 1 to 4 ring heteroatoms each independently selected from the group consisting of O, S, and NR^{d}, and then is optionally substituted on a ring carbon by 1 to 4 R^{c}; or
R⁷ and R⁹ are taken together with the carbon atoms to which they are attached to form Ring A, and R⁸ and R¹⁰ are taken together with the carbon atom and the nitrogen atom to which they are attached, respectively, to form Ring C, wherein Ring A and Ring C are each optionally substituted on a ring carbon by 1 to 4 R^{c};
R¹¹ is selected from the group consisting of H, D, C₁-C₄alkyl, C₁-C₄haloalkyl, and C₃-C₁₀cycloalkyl;
R¹² is selected from the group consisting of H, D, halo, C₁-C₄alkyl, and C₃-C₁₀cycloalkyl; or
R¹¹ and R¹² are taken together with the nitrogen atom and the carbon atom to which they are attached, respectively, to form Ring D, wherein Ring D is selected from the group consisting of a 4 to 12-membered heterocyclyl and 4 to 12-membered heteroaryl, wherein the 4 to 12-membered heterocyclyl and 4 to 12-membered heteroaryl have 1 to 4 ring heteroatoms each independently selected from the group consisting of O, S, N, and NR^{d}, and then are each optionally substituted on a ring carbon by 1 to 4 R^{c};
R¹³ is selected from the group consisting of H, D, halo, CN, C₁-C₄alkyl, C₁-C₄alkoxy, C₃-C₁₀cycloalkyl, 4 to 12-membered heterocyclyl, 4 to 12-membered aryl, and 4 to 12-membered heteroaryl, wherein the C₁-C₄alkyl, C₁-C₄alkoxy, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c};
R¹⁴ is selected from the group consisting of H, D, and C₁-C₄alkyl;
Each R^{a} is independently selected from the group consisting of H, D, halo, CN, C₁-C₄alkyl, and C₁-C₄alkoxy, wherein the C₁-C₄alkyl and C₁-C₄alkoxy each optionally substituted with 1 to 4 groups each independently selected from the group consisting of halo, OH and CN;
Each R^{b} is independently selected from the group consisting of H, D, C₁-C₄alkyl, C₃-C₁₀cycloalkyl, 4 to 12-membered heterocyclyl, 4 to 12-membered aryl, and 4 to 12-membered heteroaryl, wherein the C₁-C₄alkyl, C₃-C₁₀cycloalkyl, and 4 to 12-membered aryl are each optionally substituted with 1 to 4 groups each independently selected from the group consisting of halo, OH and CN, wherein the 4 to 12-membered heterocyclyl and 4 to 12-membered heteroaryl have 1 to 4 ring heteroatoms each independently selected from the group consisting of O, S, N, and NR^{d} and then are optionally substituted on a ring carbon with 1 to 4 groups each independently selected from the group consisting of halo, OH and CN;
Each R^{c} is independently selected from the group consisting of H, D, halo, OH, CN, C₁-C₄alkyl, and C₁-C₄alkoxy, or two R^{c}, attached to the same atom, form a-O, wherein said C₁-C₄alkyl and C₁-C₄alkoxy are each optionally substituted with 1 to 4 groups each independently selected from the group consisting of halo, OH and CN;
Each R^{d} is independently selected from the group consisting of H, D, C₁-C₄alkyl, and C(O)C₁-4alkyl; and
n is 0, 1, 2, or 3.

2. The compound of claim 1, wherein the compound is of Formula (II), (IIa), (IIb), (III), (IIIa), (IIIb), (IV), (IVa), (IVb), (V), (Va) or (Vb): 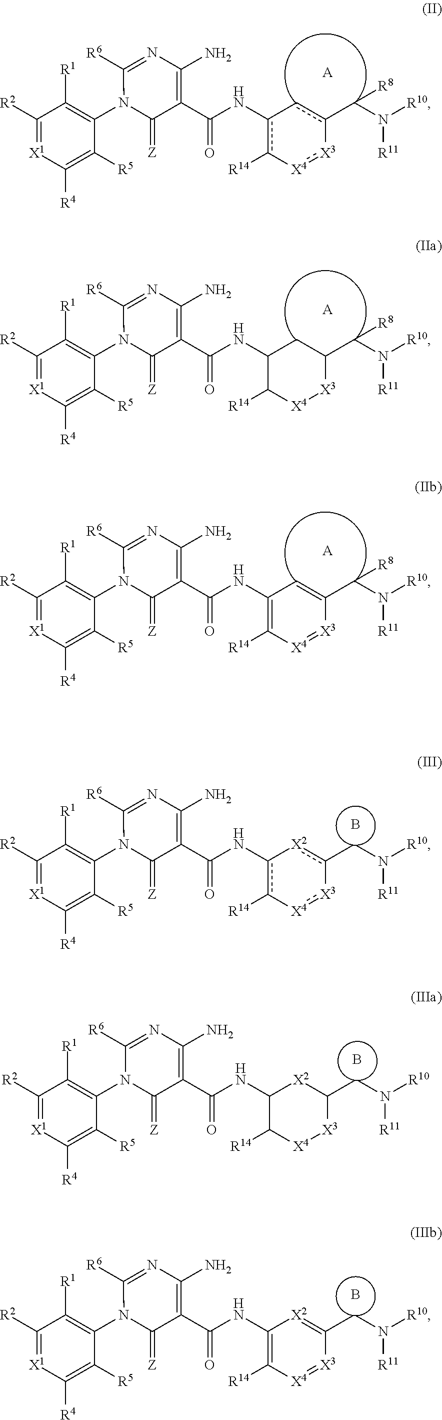 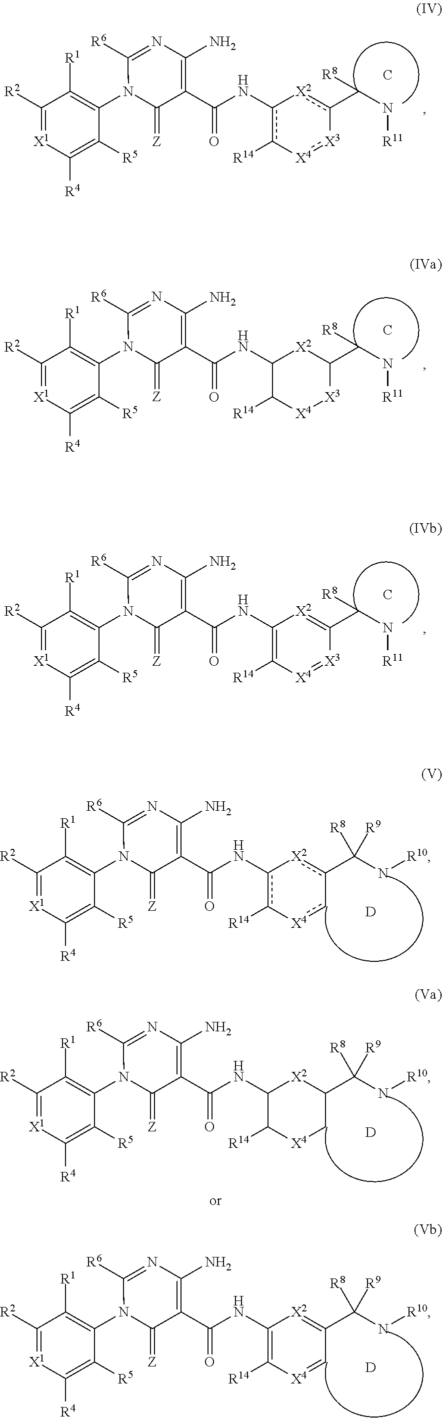
or a pharmaceutically acceptable salt thereof.

3. The compound of claim 1, wherein the compound is of Formula (IVc) or (IVd): 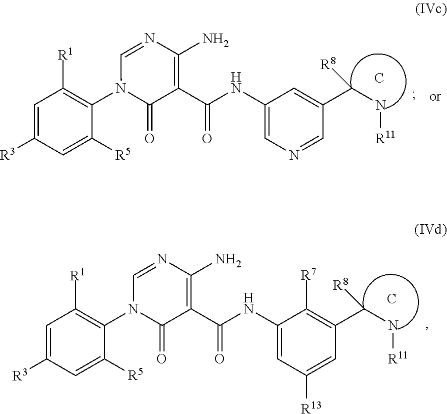
or a pharmaceutically acceptable salt thereof, wherein Ring C is a 4 to 10-membered heterocyclyl having 1 to 3 ring heteroatoms each independently selected from the group consisting of O, S, and NR^{d}, and then is optionally substituted on a ring carbon by 1 to 2 R^{c}.

4. The compound of claim 3, or a pharmaceutically acceptable salt thereof, wherein (i) Ring C is piperidine, 1,4-oxazepane, azetidine, morpholine, pyrrolidine, piperazine, piperazine-2-one, octahydrocyclopenta[c]pyrrole, or azepane, each of which is optionally substituted on a ring carbon by 1 to 2 R^{c}, or (ii) Ring C is represented by 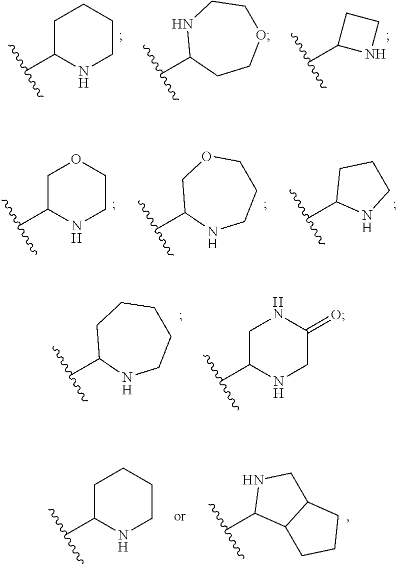
each of which is optionally substituted with 1 or 2 R^{c}; and each R^{c} is independently C₁-C₃alkyl optionally substituted with halo or OH.

5. The compound of claim 1, wherein the compound is of Formula (VI): 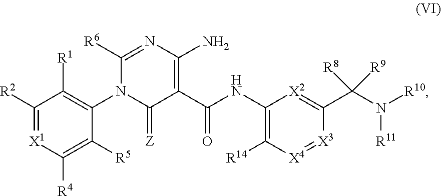
or a pharmaceutically acceptable salt thereof.

6. The compound of claim 5, or a pharmaceutically acceptable salt thereof, wherein
X¹ is CR³ or N;
X² is CR⁷ or N;
X³ is CR¹² or N;
X⁴ is CR¹³ or N;
Z is O or S;
R¹ is selected from the group consisting of halo, OH, CN, C₁-C₄alkyl, C₁-C₄alkoxy, and C₁-C₄haloalkyl;
R² is selected from the group consisting of H, D, C₁-C₄alkyl, and C₃-C₁₀cycloalkyl;
R³ is selected from the group consisting of H, D, halo, CN, C₁-C₄alkyl, —(C(R^{a})₂)ₙ—OR^{b}, —(C(R^{a})₂)ₙ—C(O)OR^{b}, —(C(R^{a})₂)ₙ—SO₂—R^{b}, —N(R^{a})₂, C₃-C₁₀cycloalkyl, 4 to 12-membered heterocyclyl, 4 to 12-membered aryl, and 4 to 12-membered heteroaryl, wherein the C₁-C₄alkyl, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c};
R⁴ is selected from the group consisting of H, D, C₁-C₄alkyl, and C₃-C₁₀cycloalkyl;
R⁵ is selected from the group consisting of halo, OH, CN, C₁-C₄alkyl, C₁-C₄alkoxy, and C₁-C₄haloalkyl;
R⁶ is H or D;
R⁷ is selected from the group consisting of H, D, halo, C₁-C₄alkyl, C₁-C₄alkoxy, C₃-C₁₀cycloalkyl, and 4 to 12-membered heterocyclyl, wherein the C₁-C₄alkyl, C₁-C₄alkoxy, and C₃-C₁₀cycloalkyl are each optionally substituted with 1 to 4 R^{c}, wherein the 4 to 12-membered heterocyclyl has 1 to 4 ring heteroatoms each independently selected from the group consisting of O, S and NR^{d} and then are each optionally substituted on a ring carbon with 1 to 4 R^{c};
R⁸ is selected from the group consisting of H, D, C₁-C₄alkyl, and C₃-C₁₀cycloalkyl;
R⁹ are each independently selected from the group consisting of H, D, C₁-C₄alkyl, C₂-C₄alkenyl, C₂-C₄alkynyl, —(C(R^{a})₂)ₙ—OR^{b}, C₃-C₁₀cycloalkyl, 4 to 12-membered heterocyclyl, 4 to 12-membered aryl, and 4 to 12-membered heteroaryl, wherein the C₁-C₄alkyl, C₂-C₄alkenyl, C₂-C₄alkynyl, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c}; or
R⁷ and R⁹ are taken together with the carbon atoms to which they are attached to form Ring A, wherein Ring A is C₃-C₁₀cycloalkyl or 4 to 12-membered heterocyclyl, wherein the C₃-C₁₀cycloalkyl is optionally substituted with 1 to 4 R^{c}, wherein the 4 to 12-membered heterocyclyl has 1 to 4 ring heteroatoms each independently selected from the group consisting of O, S, and NR^{d}, and then is optionally substituted on a ring carbon by 1 to 4 R^{c}; and/or
R⁸ and R⁹ are taken together with the carbon atom to which they are attached to form Ring B, wherein Ring B is C₃-C₁₀cycloalkyl or 4 to 12-membered heterocyclyl, wherein the C₃-C₁₀cycloalkyl is optionally substituted with 1 to 4 R^{c}, wherein the 4 to 12-membered heterocyclyl has 1 to 4 ring heteroatoms each independently selected from the group consisting of O, S, and NR^{d}, and then is optionally substituted on a ring carbon by 1 to 4 R^{c}; or
R⁸ and R⁹ are taken together form a ═O;
R¹⁰ is selected from the group consisting of H, D, C₁-C₄ alkyl, —(C(R^{a})₂)ₙ—OR^{b}, —(C(R^{a})₂)ₙ—SO₂R^{b}, C₃-C₁₀cycloalkyl, 4 to 12-membered heterocyclyl, 4 to 12-membered aryl, and 4 to 12-membered heteroaryl, wherein the C₁-C₄alkyl, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c}; or
R⁹ and R¹⁰ are taken together with the carbon atom and the nitrogen atom to which they are attached, respectively, to form Ring C, wherein Ring C is a 4 to 12-membered heterocyclyl having 1 to 4 ring heteroatoms each independently selected from the group consisting of O, S, and NR^{d}, and then is optionally substituted on a ring carbon by 1 to 4 R^{c};
R¹¹ is selected from the group consisting of H, D, C₁-C₄alkyl, and C₃-C₁₀cycloalkyl;
R¹² is selected from the group consisting of H, D, halo, C₁-C₄alkyl, and C₃-C₁₀cycloalkyl;
R¹³ is selected from the group consisting of H, D, halo, CN, C₁-C₄alkyl, C₁-C₄alkoxy, C₃-C₁₀cycloalkyl, 4 to 12-membered heterocyclyl, 4 to 12-membered aryl, and 4 to 12-membered heteroaryl, wherein the C₁-C₄alkyl, C₁-C₄alkoxy, C₃-C₁₀cycloalkyl, and 4 to 12-membered aryl are each optionally substituted with 1 to 4 R^{c}, wherein the 4 to 12-membered heterocyclyl and 4 to 12-membered heteroaryl have 1 to 4 ring heteroatoms each independently selected from the group consisting of O, S, N, and NR^{d} and then are each optionally substituted on a ring carbon with 1 to 4 R^{c};
R¹⁴ is selected from the group consisting of H, D, and C₁-C₄alkyl;
Each R^{a} is independently selected from the group consisting of H, D, halo, CN, C₁-C₄alkyl, and C₁-C₄alkoxy, wherein the C₁-C₄alkyl and C₁-C₄alkoxy each optionally substituted with 1 to 4 groups each independently selected from the group consisting of halo, OH and CN;
Each R^{b} is independently selected from the group consisting of H, D, C₁-C₄alkyl, C₃-C₁₀cycloalkyl, 4 to 12-membered heterocyclyl, 4 to 12-membered aryl, and 4 to 12-membered heteroaryl, wherein the C₁-C₄alkyl, C₃-C₁₀cycloalkyl, and 4 to 12-membered aryl are each optionally substituted with 1 to 4 groups each independently selected from the group consisting of halo, OH and CN, wherein the 4 to 12-membered heterocyclyl and 4 to 12-membered heteroaryl have 1 to 4 ring heteroatoms each independently selected from the group consisting of O, S, N, and NR^{d} and then are optionally substituted on a ring carbon with 1 to 4 groups each independently selected from the group consisting of halo, OH and CN;
Each R^{c} is independently selected from the group consisting of H, D, halo, OH, CN, C₁-C₄alkyl, and C₁-C₄alkoxy, or two R^{c}, attached to the same atom, form a-O, wherein the C₁-C₄alkyl and C₁-C₄alkoxy are each optionally substituted with 1 to 4 groups each independently selected from the group consisting of halo, OH and CN;
Each R^{d} is independently selected from the group consisting of H, D, C₁-C₄alkyl, and C(O)C₁-4alkyl; and
n is 0, 1, 2, or 3.

7. The compound of claim 5 or 6, or a pharmaceutically acceptable salt thereof, wherein X¹ is CR³ or N;
X² is CR⁷ or N;
X³ is CR¹² or N;
X⁴ is CR¹³ or N;
Z is O;
R¹ is selected from the group consisting of halo, OH, C₁-C₄alkyl, C₁-C₄alkoxy, and C₁-C₄haloalkyl;
R² is selected from the group consisting of H, D, and C₁-C₄alkyl;
R³ is selected from the group consisting of H, D, halo, CN, C₁-C₄alkyl, —(C(R^{a})₂)ₙ—OR^{b}, —(C(R^{a})₂)ₙ—C(O)OR^{b}, —(C(R^{a})₂)ₙ—SO₂—R^{b}, —N(R^{a})₂, C₃-C₈cycloalkyl, 4 to 10-membered heterocyclyl, 4 to 10-membered aryl, and 4 to 10-membered heteroaryl, wherein the C₁-C₄alkyl, C₃-C₈cycloalkyl, and 4 to 10-membered aryl are each optionally substituted with 1 to 3 R^{c}, wherein the 4 to 10-membered heterocyclyl and 4 to 10-membered heteroaryl have 1 to 3 ring heteroatoms each independently selected from the group consisting of O, S, N, and NR^{d} and then are each optionally substituted on a ring carbon with 1 to 3 R^{c};
R⁴ is selected from the group consisting of H, D, and C₁-C₄alkyl;
R⁵ is selected from the group consisting of halo, OH, C₁-C₄alkyl, C₁-C₄alkoxy, and C₁-C₄haloalkyl;
R⁶ is H or D;
R⁷ is selected from the group consisting of H, D, halo, C₁-C₄alkyl, C₁-C₄alkoxy, C₃-C₈cycloalkyl, and 4 to 10-membered heterocyclyl, wherein the C₁-C₄alkyl, C₁-C₄alkoxy, and C₃-C₈cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are each optionally substituted on a ring carbon with 1 to 3 R^{c};
R⁸ is selected from the group consisting of H, D, and C₁-C₄alkyl;
R⁹ are each independently selected from the group consisting of H, D, C₁-C₄alkyl, C₂-C₄alkenyl, C₂-C₄alkynyl, —(C(R^{a})₂)ₙ—OR^{b}, C₃-C₈cycloalkyl, and 4 to 10-membered heterocyclyl, wherein the C₁-C₄alkyl, C₂-C₄alkenyl, C₂-C₄alkynyl, and C₃-C₈cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are each optionally substituted on a ring carbon with 1 to 3 R^{c}; or
R⁷ and R⁹ are taken together with the carbon atoms to which they are attached to form Ring A, wherein Ring A is C₃-C₈cycloalkyl or 4 to 10-membered heterocyclyl, wherein the C₃-C₈cycloalkyl is optionally substituted with 1 to 3 R^{c}, wherein the 4 to 10-membered heterocyclyl has 1 to 3 ring heteroatoms each independently selected from the group consisting of O, S, and NR^{d}, and then is optionally substituted on a ring carbon by 1 to 3 R^{c}; and/or
R⁸ and R⁹ are taken together with the carbon atom to which they are attached to form Ring B, wherein Ring B is C₃-C₈cycloalkyl or 4 to 10-membered heterocyclyl, wherein the C₃-C₈cycloalkyl is optionally substituted with 1 to 3 R^{c}, wherein the 4 to 10-membered heterocyclyl has 1 to 3 ring heteroatoms each independently selected from the group consisting of O, S, and NR^{d}, and then is optionally substituted on a ring carbon by 1 to 3 R^{c}; or
R⁸ and R⁹ are taken together form a ═O;
R¹⁰ is selected from the group consisting of H, D, C₁-C₄ alkyl, —(C(R^{a})₂)ₙ—OR^{b}, —(C(R^{a})₂)ₙ—SO₂R^{b}, C₃-C₈cycloalkyl, 4 to 10-membered heterocyclyl, 4 to 10-membered aryl, and 4 to 10-membered heteroaryl, wherein the C₁-C₄alkyl, C₃-C₈cycloalkyl, and 4 to 10-membered aryl are each optionally substituted with 1 to 3 R^{c}, wherein the 4 to 10-membered heterocyclyl and 4 to 10-membered heteroaryl have 1 to 3 ring heteroatoms each independently selected from the group consisting of O, S, N, and NR^{d} and then are each optionally substituted on a ring carbon with 1 to 3 R^{c}; or
R⁹ and R¹⁰ are taken together with the carbon atom and the nitrogen atom to which they are attached, respectively, to form Ring C, wherein Ring C is a 4 to 10-membered heterocyclyl having 1 to 3 ring heteroatoms each independently selected from the group consisting of O, S, and NR^{d}, and then is optionally substituted on a ring carbon by 1 to 3 R^{c};
R¹¹ is selected from the group consisting of H, D, and C₁-C₄alkyl;
R¹² is selected from the group consisting of H, D, halo, and C₁-C₄alkyl;
R¹³ is selected from the group consisting of H, D, halo, CN, C₁-C₄alkyl, and C₁-C₄alkoxy, wherein the C₁-C₄alkyl and C₁-C₄alkoxy are each optionally substituted with 1 to 3 R^{c};
R¹⁴ is selected from the group consisting of H, D, and C₁-C₄alkyl;
Each R^{a} is independently selected from the group consisting of H, D, halo, CN, C₁-C₄alkyl, and C₁-C₄alkoxy, wherein the C₁-C₄alkyl and C₁-C₄alkoxy each optionally substituted with 1 to 3 groups each independently selected from the group consisting of halo, OH and CN;
Each R^{b} is independently selected from the group consisting of H, D, C₁-C₄alkyl, C₃-C₈cycloalkyl, 4 to 10-membered heterocyclyl, 4 to 10-membered aryl, and 4 to 10-membered heteroaryl, wherein the C₁-C₄alkyl, C₃-C₈cycloalkyl, and 4 to 10-membered aryl are each optionally substituted with 1 to 3 groups each independently selected from the group consisting of halo, OH and CN, wherein the 4 to 10-membered heterocyclyl and 4 to 10-membered heteroaryl have 1 to 3 ring heteroatoms each independently selected from the group consisting of O, S, N, and NR^{d} and then are optionally substituted on a ring carbon with 1 to 3 groups each independently selected from the group consisting of halo, OH and CN;
Each R^{c} is independently selected from the group consisting of H, D, halo, OH, CN, C₁-C₄alkyl, and C₁-C₄alkoxy, or two R^{c}, attached to the same atom, form a-O, wherein the C₁-C₄alkyl and C₁-C₄alkoxy are each optionally substituted with 1 to 3 groups each independently selected from the group consisting of halo, OH and CN;
Each R^{d} is independently selected from the group consisting of H, D, C₁-C₄alkyl, and C(O)C₁-4alkyl; and
n is 0, 1, 2, or 3.

8. The compound of claim 1 or 5, wherein the compound is of Formula (VIa), (VIb), (VIc), (VId), (VIe), (VIf), (VIg), or (VIh): 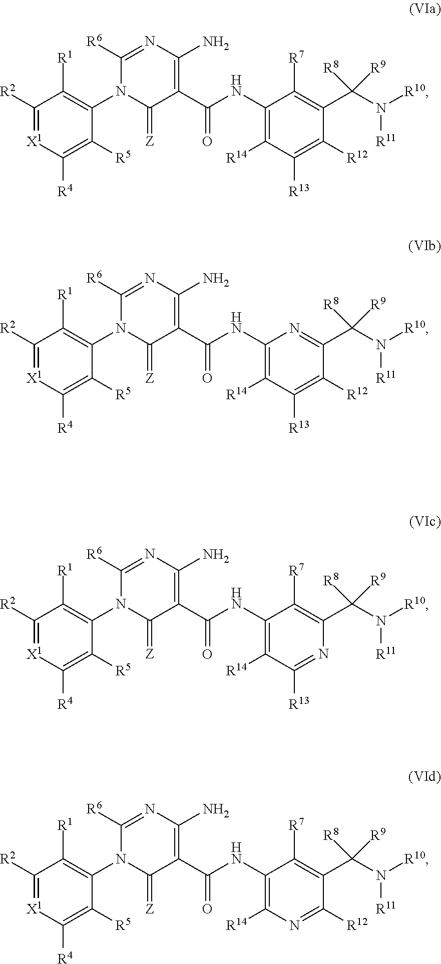 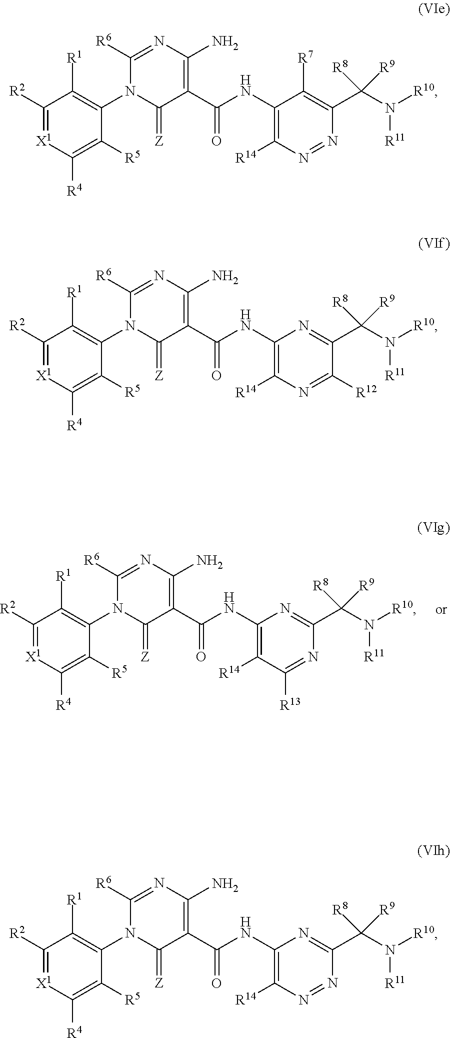
or a pharmaceutically acceptable salt thereof.

9. The compound of claim 1, wherein the compound is of Formula (VII), (VIIa) or (VIIb): 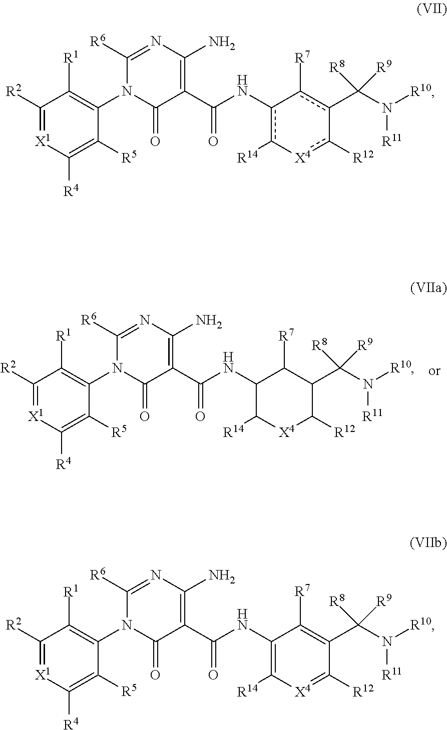
or a pharmaceutically acceptable salt thereof.

10. The compound of claim 1 or 9, or a pharmaceutically acceptable salt thereof, wherein
X¹ is CR³ or N;
X⁴ is selected from the group consisting of CR¹³, N, and NR^{d},
R¹ is selected from the group consisting of halo, C₁-C₄alkyl, and C₁-C₄haloalkyl;
R² is selected from the group consisting of H, D, and C₁-C₄alkyl;
R³ is selected from the group consisting of H, D, halo, CN, C₁-C₄alkyl, —(C(R^{a})₂)ₙ—OR^{b}, —(C(R^{a})₂)ₙ—C(O)OR^{b}, —(C(R^{a})₂)ₙ—SO₂—R^{b}, —N(R^{a})₂, C₃-C₈cycloalkyl, and 4 to 10-membered heterocyclyl, wherein the C₁-C₄alkyl and C₃-C₁₀cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are optionally substituted on a ring carbon with 1 to 3 R^{c};
R⁴ is selected from the group consisting of H, D, and C₁-C₄alkyl;
R⁵ is selected from the group consisting of halo, C₁-C₄alkyl, and C₁-C₄haloalkyl;
R⁶ is H or D;
R⁷ is selected from the group consisting of H, D, halo, C₁-C₄alkyl, and C₁-C₄alkoxy, C₃-C₆cycloalkyl, and 4 to 7-membered heterocyclyl, wherein the C₁-C₄alkyl, C₁-C₄alkoxy, and C₃-C₆cycloalkyl are each optionally substituted with 1 to 3 R^{c}, wherein the 4 to 7-membered heterocyclyl has 1 to 3 ring heteroatoms each independently selected from the group consisting of O, S, and NR^{d} and then are each optionally substituted on a ring carbon with 1 to 3 R^{c};
R⁸ is selected from the group consisting of H, D, and C₁-C₄alkyl;
R⁹ are each independently selected from the group consisting of H, D, C₁-C₄alkyl, C₂-C₄alkynyl, —(C(R^{a})₂)ₙ—OR^{b}, C₃-C₈cycloalkyl, and 4 to 10-membered heterocyclyl, wherein the C₁-C₄alkyl, C₂-C₄alkynyl, and C₃-C₈cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are optionally substituted on a ring carbon with 1 to 3 R^{c}; or
R⁸ and R⁹ are taken together with the carbon atom to which they are attached to form Ring B, wherein Ring B is C₃-C₈cycloalkyl or 4 to 10-membered heterocyclyl, wherein the C₃-C₈cycloalkyl is optionally substituted with 1 to 3 R^{c}, wherein the 4 to 10-membered heterocyclyl has 1 to 3 ring heteroatoms each independently selected from the group consisting of O, S, and NR^{d}, and then is optionally substituted on a ring carbon by 1 to 3 R^{c}; or
R⁸ and R⁹ are taken together form a ═O;
R¹⁰ is selected from the group consisting of H, D, C₁-C₄ alkyl, —(C(R^{a})₂)ₙ—OR^{b}, —(C(R^{a})₂)ₙ—SO₂R^{b}, C₃-C₈cycloalkyl, and 4 to 10-membered heterocyclyl, wherein the C₁-C₄ alkyl and C₃-C₈cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are optionally substituted on a ring carbon with 1 to 3 Re; or
R⁹ and R¹⁰ are taken together with the carbon atom and the nitrogen atom to which they are attached, respectively, to form Ring C, wherein Ring C is a 4 to 10-membered heterocyclyl having 1 to 3 ring heteroatoms each independently selected from the group consisting of O, S, and NR^{d}, and then is optionally substituted on a ring carbon by 1 to 3 R^{c};
R¹¹ is selected from the group consisting of H, D, and C₁-C₄alkyl;
R¹² is selected from the group consisting of H, D, halo, and C₁-C₄alkyl;
R¹³ is selected from the group consisting of H, D, halo, CN, and C₁-C₄alkyl;
R¹⁴ is selected from the group consisting of H, D, and C₁-C₄alkyl;
Each R^{a} is independently selected from the group consisting of H, D, and C₁-C₄alkyl;
Each R^{b} is independently selected from the group consisting of H, D, C₁-C₄alkyl, C₁-C₄alkoxy, C₃-C₈cycloalkyl, and 4 to 10-membered heterocyclyl, wherein the C₁-C₄alkyl and C₃-C₈cycloalkyl are each optionally substituted with 1 to 3 groups each independently selected from the group consisting of halo, OH and CN, wherein the 4 to 10-membered heterocyclyl has 1 to 3 ring heteroatoms each independently selected from the group consisting of O, S, and NR^{d} and then are optionally substituted on a ring atom with 1 to 3 groups each independently selected from the group consisting of halo, OH and CN;
Each R^{c} is independently selected from the group consisting of D, halo, OH, CN, C₁-C₄alkyl, and C₁-C₄alkoxy, or two R^{a}, attached to the same atom, form a ═O, wherein the C₁-C₄alkyl and C₁-C₄alkoxy are each optionally substituted with 1 to 3 groups each independently selected from the group consisting of halo, OH and CN;
Each R^{d} is independently selected from the group consisting of H, D, C₁-C₄alkyl, and C(O)C₁-4alkyl; and
n is 0, 1, 2, or 3.

11. The compound of claim 1 or 9, wherein the compound is of Formula (VIIIe), (VIIIf), (VIIIg) or (VIIIh): 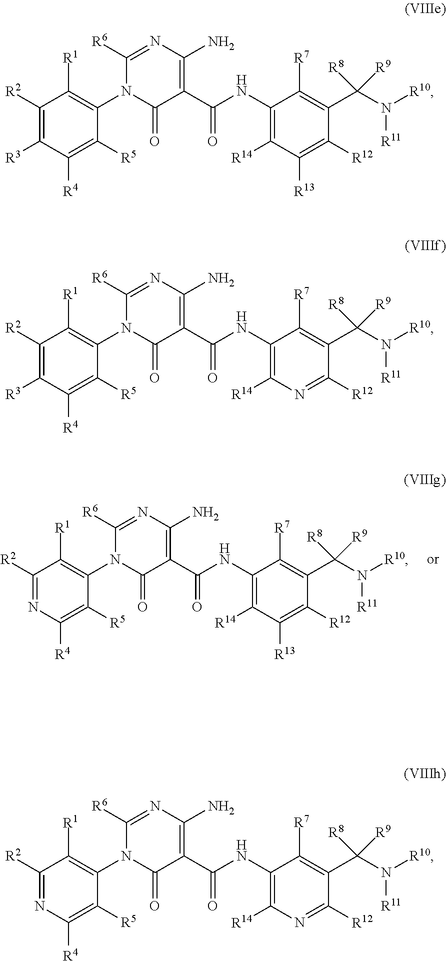
or a pharmaceutically acceptable salt thereof.

12. The compound of any one of claims 1-11, or a pharmaceutically acceptable salt thereof, wherein: (i) R¹ is halo, —OH, C₁-C₄alkyl, or C₁-C₄alkoxy; or (ii) R¹ is —CH₃, —Cl, —OH, or —OMe.

13. The compound of any one of claims 1-12, or a pharmaceutically acceptable salt thereof, wherein: (i) R² is H, D, halo, or C₁-C₄alkyl; or (ii) R² is H.

14. The compound of any one of claims 1-13, or a pharmaceutically acceptable salt thereof, wherein: (i) R³ is selected from the group consisting of H, D, halo, CN, C₁-C₄alkyl, —(C(R^{a})₂)ₙ—OR^{b}, —(C(R^{a})₂)ₙ—C(O)OR^{b}, —(C(R^{a})₂)ₙ—SO₂—R^{b}, —N(R^{a})₂, C₃-C₆cycloalkyl, 4 to 7-membered heterocyclyl, phenyl, and 4 to 6-membered heteroaryl, wherein the C₁-C₄alkyl, C₃-C₆cycloalkyl, and phenyl are each optionally substituted with 1 to 4 R^{c}, wherein the 4 to 7-membered heterocyclyl and 4 to 6-membered heteroaryl have 1 to 3 ring heteroatoms each independently selected from the group consisting of O, S, N, and NR^{d} and then are each optionally substituted on a ring carbon with 1 to 3 R^{c}; (ii) R³ is selected from the group consisting of —CH₂CH₃, —CH₂OCH₃, —CF₃, —OCH₃, H, —CN, —CHF₂, —CHFCH₃, —CHF—CH₂F, —CH(CH₃)OMe, —OCHF₂, C₁, —OCH₂CH₃, F, —CH₃, —O-cyclopropyl, —O—CH₂CH₂CH₃, —O—CF₃, —N(CH₃)₂, —O—CH(CH₃)₂, —CH₂OCH₂CH₃, —CH₂CHF₂, —CH₂CH₂F, —CH₂SO₂CH₃, —CF₂Me, —C(═O)OCH₃, —C(═O)OCH₂CH₂CH₃, —CH₂CF₃, and —CH₂CO₂H, or is represented by one of the following structures: 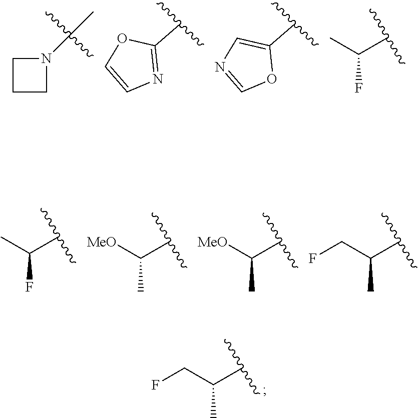
or (iii) R³ is —CH₂CH₃, —CH₂OCH₃, —CF₃, or —OCH₃.

15. The compound of any one of claims 1-14, or a pharmaceutically acceptable salt thereof, wherein: (i) R⁴ is H, D, halo, or C₁-C₄alkyl; (ii) R⁴ is H.

16. The compound of any one of claims 1-15, or a pharmaceutically acceptable salt thereof, wherein: (i) R⁵ is halo, OH, C₁-C₄alkyl, C₁-C₄haloalkyl or C₁-C₄alkoxy; or (ii) R⁵ is Me, C₁, —OH, —CHF₂ or —OCH₃.

17. The compound of any one of claims 1-16, or a pharmaceutically acceptable salt thereof, wherein: (i) R⁷ is H, D, halo, C₁-C₄alkyl, C₁-C₄alkoxy, or 4 to 7-membered heterocyclyl, wherein the C₁-C₄alkyl, and C₁-C₄alkoxy are each optionally substituted with 1 to 3 R^{c}, wherein the 4 to 7-membered heterocyclyl has 1 to 3 ring heteroatoms each independently selected from the group consisting of O, S, and NR^{d} and then are each optionally substituted on a ring carbon with 1 to 3 Re; (ii) R⁷ is H, morpholin-N-yl, —CF₃, —F, —OCHF₂, —OCH₂CH₃, or —OCH₃; or (iii) R⁷ is H.

18. The compound of any one of claims 1-17, or a pharmaceutically acceptable salt thereof, wherein: (i) R⁸ is H, D, halo, or C₁-C₄alkyl; (ii) R⁸ is H or —CH₃; or (ii) R⁸ is H.

19. The compound of any one of claims 1-18, or a pharmaceutically acceptable salt thereof, wherein: (i) R⁹ is each independently H, D, C₁-C₄alkyl, C₂-C₄alkynyl, —(C(R^{a})₂)ₙ—OR^{b}, or C₃-C₈cycloalkyl, wherein the C₁-C₄alkyl, C₂-C₄alkynyl, and C₃-C₈cycloalkyl are each optionally substituted with 1 to 3 R^{c}; (ii) R⁹ is each independently H, Me, —CH₂OCH₂CH₃, —CH₂CH₂CH₃, —CH₂CH₃, —CH₂OCH₃, —CH₂CHF₂, cyclopropyl, —CH₂C═CH, —CH₂CH₂OCH₃, or —CHF₂; or (iii) R⁹ is each independently H, Me, —CH₂OCH₂CH₃, —CH₂CH₂CH₃, —CH₂CH₃, or —CH₂OCH₃.

20. The compound of any one of claims 1-19, or a pharmaceutically acceptable salt thereof, wherein: (i) R¹⁰ is H, D, C₁-C₄ alkyl, —(C(R^{a})₂)ₙ—OR^{b}, —(C(R^{a})₂)ₙ—SO₂R^{b}, or C₃-C₈cycloalkyl, wherein the C₁-C₄ alkyl and C₃-C₈cycloalkyl are each optionally substituted with 1 to 3 R^{c}; (ii) R¹⁰ is H, —CH₃, —CH₂CH₃, —CH₂CH₂CH₃, —CH₂CH₂SO₂CH₃, —CD₂CD₃, —CH₂CH₂OH, —CH₂CH₂OCH₃, —CH(CH₃)₂, or —CD₃; or (iii) R¹⁰ is H, —CH₃, or —CH₂CH₃.

21. The compound of any one of claims 1-20, or a pharmaceutically acceptable salt thereof, wherein: (i) R¹¹ is H, D, halo, or C₁-C₄alkyl; or (ii) R¹¹ is H.

22. The compound of any one of claims 1-21, or a pharmaceutically acceptable salt thereof, wherein: (i) R¹² is H, D, halo or C₁-C₄alkyl; (ii) R¹² is H, D, or halo; (iii) R¹² is H or F; or (iv) R¹² is H.

23. The compound of any one of claims 1-22, or a pharmaceutically acceptable salt thereof, wherein: (i) R¹³ is H, D, halo or C₁-C₄alkyl; (ii) R¹³ is H, D, or halo; (iii) R¹³ is H or F; or (iv) R¹³ is H.

24. The compound of any one of claims 1-23, or a pharmaceutically acceptable salt thereof, wherein R¹⁴ is H.

25. The compound of claim 1, wherein the compound is of Formula (IXa) or (IXb): 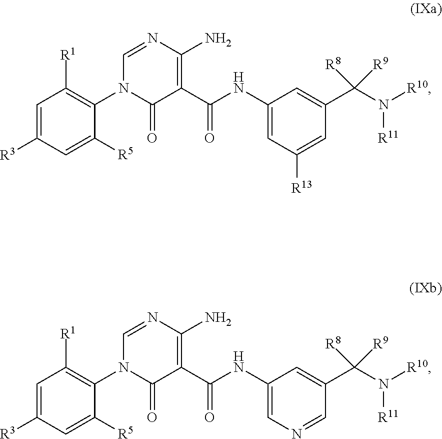
or a pharmaceutically acceptable salt thereof, wherein
R¹ is selected from the group consisting of halo, C₁-C₄alkyl, and C₁-C₄haloalkyl;
R³ is selected from the group consisting of H, D, halo, CN, C₁-C₄alkyl, —(C(R^{a})₂)ₙ—OR^{b}, —(C(R^{a})₂)ₙ—C(O)OR^{b}, —(C(R^{a})₂)ₙ—SO₂—R^{b}, —N(R^{a})₂, C₃-C₈cycloalkyl, and 4 to 10-membered heterocyclyl, wherein the C₁-C₄alkyl and C₃-C₈cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are optionally substituted on a ring carbon with 1 to 3 R^{c};
R⁵ is selected from the group consisting of halo, C₁-C₄alkyl, and C₁-C₄haloalkyl;
R⁸ is selected from the group consisting of H, D, and C₁-C₄alkyl;
R⁹ is selected from the group consisting of H, D, C₁-C₄alkyl, —(C(R^{a})₂)ₙ—OR^{b}, C₃-C₈cycloalkyl, and 4 to 10-membered heterocyclyl, wherein the C₁-C₄alkyl and C₃-C₈cycloalkyl are each optionally substituted with 1 to 3 R^{c}, wherein the 4 to 10-membered heterocyclyl has 1 to 3 ring heteroatoms each independently selected from the group consisting of O, S, and NR^{d} and then are optionally substituted on a ring carbon with 1 to 3 R^{c};
R¹⁰ is selected from the group consisting of H, D, C₁-C₄ alkyl, C₃-C₁₀cycloalkyl, and 4 to 12-membered heterocyclyl, wherein the C₁-C₄ alkyl and C₃-C₁₀cycloalkyl are each optionally substituted with 1 to 4 R^{c}, wherein the 4 to 12-membered heterocyclyl has 1 to 4 ring heteroatoms each independently selected from the group consisting of O, S, and NR^{d} and then are optionally substituted on a ring carbon with 1 to 4 R^{c}; or
R⁹ and R¹⁰ are taken together with the carbon atom and the nitrogen atom to which they are attached, respectively, to form Ring C, wherein Ring C is a 4 to 10-membered heterocyclyl having 1 to 3 ring heteroatoms each independently selected from the group consisting of O, S, and NR^{d}, and then is optionally substituted on a ring carbon by 1 to 3 R^{c};
R¹¹ is selected from the group consisting of H, D, and C₁-C₄alkyl;
R¹³ is selected from the group consisting of H, D, halo, CN, and C₁-C₄alkyl;
Each R^{a} is independently selected from the group consisting of H, D, and C₁-C₄alkyl;
Each R^{b} is independently selected from the group consisting of H, D, C₁-C₄alkyl, and C₁-C₄alkoxy;
Each R^{c} is independently selected from the group consisting of D, halo, OH, CN, C₁-C₄alkyl, and C₁-C₄alkoxy, or two R^{a}, attached to the same atom, form a ═O; and
Each R^{d} is independently selected from the group consisting of H, D, and C₁-C₄alkyl; and
n is 0, 1, or 2.

26. The compound of claim 25, or a pharmaceutically acceptable salt thereof, wherein R¹ is halo or C₁-C₃alkyl;
R³ is C₁-C₃alkyl or —(C(R^{a})₂)ₙ—OR^{b}, wherein n is 0, and R^{b} is C₁-C₃alkyl, wherein the C₁-C₃alkyl is optionally substituted with 1 to 3 R^{c}, wherein R^{c} is independently halo or C₁-C₃alkoxy;
R⁵ is halo or C₁-C₃alkyl;
R⁸ is H;
R⁹ is C₁-C₃alkyl or —(C(R^{a})₂)ₙ—OR^{b}, wherein n is 0, and R^{b} is C₁-C₃alkyl, wherein the C₁-C₃alkyl is optionally substituted with 1 to 3 R^{c}, wherein R^{c} is independently halo or C₁-C₃alkoxy;
R¹⁰ is H or C₁-C₃alkyl; or
R⁹ and R¹⁰ are taken together with the carbon atom and the nitrogen atom to which they are attached, respectively, to form Ring C, wherein Ring C is a 4 to 6-membered heterocyclyl;
R¹¹ is H; and
R¹³ is H.

27. The compound of claim 25, or a pharmaceutically acceptable salt thereof, wherein
R¹ is —CH₃ or —C₁;
R³ is —CH₂CH₃, —CH₂OCH₃, —CF₃ or —OCH₃;
R⁵ is —CH₃ or —C₁;
R⁸ is H;
R⁹ is H, —CH₃, —CH₂OCH₂CH₃, —CH₂CH₂CH₃, —CH₂CH₃, or —CH₂OCH₃;
R¹⁰ is H, —CH₃, or —CH₂CH₃; or
R⁹ and R¹⁰ are taken together with the carbon atom and the nitrogen atom to which they are attached, respectively, to form Ring C, wherein Ring C is represented by 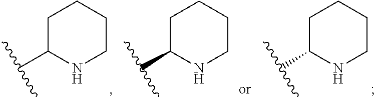
R¹¹ is H; and
R¹³ is H.

28. A pharmaceutical composition comprising: i) the compound of any one of claims 1-27 or a pharmaceutically acceptable salt thereof; and ii) a pharmaceutically acceptable carrier, excipient or diluent.

29. A method of treating a subject with a disease or disorder responsive to inhibition of glycogen synthase kinase 3 (GSK3) activity, comprising administering to the subject an effective amount of the compound of claims 1-27 or a pharmaceutically acceptable salt thereof or the pharmaceutical composition of claim 28.

30. The method of claim 29, wherein the disease or disorder is diabetes, cancer, viral infections or a CNS disorder.

31. The method of claim 29, wherein the disease or disorder is cancer.
